(19) United States
(12) Patent Application Publication
(10) Pub. No.: US 2025/0179196 A1
(43) Pub. Date: Jun. 5, 2025

(54) MATERIALS AND METHODS OF MAKING OR USING IL-23R BINDING PROTEINS

(71) Applicant: Janssen Biotech, Inc., Horsham, PA (US)

(72) Inventors: Qiang Chen, San Diego, CA (US); Ellen Chi, Del Mar, CA (US); Wilson Edwards, Cardiff, CA (US); Carrie N. Greving, San Marcos, CA (US); Ann LaCombe, San Diego, CA (US); Xinyi Li, San Diego, CA (US); Wai Lin, San Diego, CA (US)

(21) Appl. No.: 18/293,215

(22) PCT Filed: Aug. 1, 2022
(86) PCT No.: PCT/US2022/039044

§ 371 Date: Jan. 29, 2024

Related U.S. Application Data
(60) Provisional application No. 63/227,976, filed on Jul. 30, 2021.

(30) Foreign Application Priority Data
Jul. 29, 2022 (TW) 111128678

(51) Int. Cl. C07K 16/28

(52) U.S. Cl. CPC C07K 16/2866; C07K 2317/33; C07K 2317/92

(57) ABSTRACT

The present invention relates to monoclonal anti-interleukin-23 receptor (IL-23R) antibodies, nucleic acids and expression vectors encoding the antibodies, recombinant cells containing the vectors, and corresponding compositions and methods of making and using antibodies respectively, i.e., e.g., detecting, selecting, enriching, inhibiting, or antagonizing IL-23R or treat an autoimmune or inflammatory diseases or disorders.

### CROSS-REFERENCE TO RELATED APPLICATION

This application is a 371 national of PCT/US2022/039044 filed on Aug. 1, 2022, pending, which claims the benefit under 35 U.S.C. § 119 of U.S. Provisional Application No. 63/227,976, filed Jul. 30, 2021.

### INCORPORATION OF SEQUENCE LISTING

The sequence listing in ST.26 XML format entitled 2948-32-WO-ST26.xml, created on Jul. 28, 2022, comprising 2,022,624 bytes, prepared according to 37 CFR 1.822 to 1.824, submitted concurrently with the filing of this application, is incorporated herein by reference in its entirety.

### FIELD OF THE INVENTION

The present invention relates to monoclonal anti-interleukin-23 receptor (IL-23R) antibodies, nucleic acids and expression vectors encoding the antibodies, recombinant cells containing the vectors, and corresponding compositions and methods of making and using antibodiesrespectively, i.e., e.g., for detecting, selecting, enriching, inhibiting, or antagonizing IL-23R or treat a autoimmune, inflammatory diseases or disorders.

### BACKGROUND

IL-23 is a disulfide-linked heterodimer of the IL-23p19 and IL-12/23p40 subunits. The receptor for IL-23 comprises the IL-23R and IL-12RP1 subunits. IL-23p19 binding to the N-terminal immunoglobulin (Ig)-like domain of IL-23R is followed by IL-12/23p40 binding to IL-12RP1 (Bloch, Y. et al., Immunity 48: 45-58 e6, 2018). Ligand binding results in the phosphorylation of JAK2 and TYK2 followed by phosphorylation and nuclear translocation of STAT proteins. Although multiple STAT proteins are phosphorylated in response to IL-23, STAT3 is thought to play a predominant role in mediating IL-23 signaling (Parham, C. et al., J Immunol 168: 5699-708, 2002).

The IL-23 pathway has been validated as a key driver in multiple inflammatory diseases through highly effective antibody therapeutics that block IL-23 signaling (reviewed by Gaffen et al., Nat Rev Immunol 14: 585-600, 2014). IL-23 and IL-23R protein are expressed at low levels in vivo, and using currently available IL-23R detection reagents, increased IL-23R expression is not always detectable in diseases, even in those diseases where IL-23 blockade is highly efficacious. To date, there are no commercially available anti-IL-23R antibodies validated for the detection of IL-23-responsive cells in healthy individuals and disease patients or for studies of IL-23R regulation, trafficking, and turn-over at the cellular level. This is well-recognized as a major challenge to research in the field by academic and pharmaceutical industry scientists. Thus, there is a need in the art for improved anti-IL-23R antibodies for use in receptor detection and other purposes, including in applications to and methods for disease detection, management and treatment.

### SUMMARY

In general, the present invention relates to monoclonal anti-interleukin-23 receptor (IL-23R) antibodies, nucleic acids and expression vectors encoding the antibodies, recombinant cells containing the vectors, and corresponding compositions and methods of making and using antibodies respectively, i.e., e.g., for detecting, selecting, enriching, inhibiting, or antagonizing IL-23R or treating autoimmune, inflammatory diseases or disorders.

In a general aspect, the invention provides novel interleukin-23 receptor (IL-23R) antibodies directed against IL-23R, particularly human IL-23R. In an aspect, provided herein are isolated antibodies, particularly an antibody or fragment thereof, including an Fab fragment, a single chain or domain antibody, which specifically recognizes IL-23R. In an aspect, provided herein are antodies or antigen binding fragments thereof, wherein the antigen binding fragment is capable of binding to or recognizing IL-23R, particularly human IL-23R.

In an aspect, the antibody or antigen binding fragment thereof binds to or specifically recognizes extracellular domain (ECD) of IL-23R. In an aspect, the antibody or antigen binding fragment thereof binds human interleukin-23 receptor (IL-23R) on a mammalian cell. In an aspect, the antibody or antigen binding fragment thereof binds human interleukin-23 receptor (IL-23R) on human immune cells. In an aspect, the antibody or antigen binding fragment thereof binds human interleukin-23 receptor (IL-23R) on primary human immune cells. In an aspect, the antibody or antigen binding fragment thereof binds human interleukin-23 receptor (IL-23R) on the surface of cells including primary human immune cells. In an aspect, the antibody or antigen binding fragment thereof binds human interleukin-23 receptor (IL-23R) on T cells. In one aspect, the antibody or antigen binding fragment thereof binds to or specifically recognizes human IL-23R and also rat IL-23R. In one aspect, the antibody or antigen binding fragment thereof binds to or specifically recognizes human IL-23R ECD and also rat IL-23R ECD.

In some embodiments, the antibody or antigen binding fragment thereof binds human interleukin-23 receptor (IL-23R) on a mammalian cell. In embodiments, the antibody that binds IL23R on the surface of cells including primary immune cells comprises or is selected from antibody designated herein as I23RB1, I23RB102, I23RB148, I23RB155, I23RB156, I23RB157, I23RB160, 123RB162, I23RB165, 123RB166, 123RB173, I23RB2, I23RB27, I23RB270, I23RB3, I23RB30, I23RB318, I23RB32, I23RB33, I23RB36, I23RB39, I23RB4, I23RB42, I23RB45, I23RB47, I23RB58, I23RB65, I23RB7, I23RB71, I23RB73, I23RB74, I23RB76, I23RB77, I23RB81, I23RB82, I23RB86, I23RB92, I23RB93 or I23RB94. In an embodiment, the mammalian cell is a human cell or a rat cell. In an embodiment, the cell is an immune cell. In an embodiment, the cell is a primary immune cell. In an embodiment, the cell is a human primary immune cell or a rat primary immune cell. In an embodiment, the cell is a human T cell or a rat T cell. In embodiments, the cell(s) may be or may include include αβ T cells, including CD4+ and CD8+ T cells, γδ T cells, natural killer (NK) cells, innate lymphoid cells (ILCs), or monocyte-derived macrophages. In embodiments, the cell(s) may be or may include include αβ T cells, including CD4+ and CD8+ T cells, or γδ T cells. In some embodiments, the cell is a natural killer (NK) cell. In some embodiments, the cell is an innate lymphoid cell (ILC). In some embodiments, the cell is a monocyte-derived macrophage.

In an embodiment, an isolated monoclonal antibody or active fragment thereof is provided that binds human interleukin-23 receptor (IL-23R) on a mammalian cell, wherein the antibody or fragment comprises:
a. an HCDR1, an HCDR2, and an HCDR3 as set forth in SEQ ID NO:236, and an LCDR1, an LCDR2, and an LCDR3 as set forth in SEQ ID NO:237;
b. an HCDR1, an HCDR2, and an HCDR3 as set forth in SEQ ID NO:798, and an LCDR1, an LCDR2, and an LCDR3 as set forth in SEQ ID NO:799;
c. an HCDR1, an HCDR2, and an HCDR3 as set forth in SEQ ID NO:792, and an LCDR1, an LCDR2, and an LCDR3 as set forth in SEQ ID NO:793;
d. an HCDR1, an HCDR2, and an HCDR3 as set forth in SEQ ID NO:786, and an LCDR1, an LCDR2, and an LCDR3 as set forth in SEQ ID NO:787;
e. an HCDR1, an HCDR2, and an HCDR3 as set forth in SEQ ID NO:138, and an LCDR1, an LCDR2, and an LCDR3 as set forth in SEQ ID NO:139;
f. an HCDR1, an HCDR2, and an HCDR3 as set forth in SEQ ID NO:144, and an LCDR1, an LCDR2, and an LCDR3 as set forth in SEQ ID NO:145;
g. an HCDR1, an HCDR2, and an HCDR3 as set forth in SEQ ID NO:152, and an LCDR1, an LCDR2, and an LCDR3 as set forth in SEQ ID NO:153;
h. an HCDR1, an HCDR2, and an HCDR3 as set forth in SEQ ID NO:154, and an LCDR1, an LCDR2, and an LCDR3 as set forth in SEQ ID NO:155;
i. an HCDR1, an HCDR2, and an HCDR3 as set forth in SEQ ID NO:156, and an LCDR1, an LCDR2, and an LCDR3 as set forth in SEQ ID NO:157;
j. an HCDR1, an HCDR2, and an HCDR3 as set forth in SEQ ID NO:160, and an LCDR1, an LCDR2, and an LCDR3 as set forth in SEQ ID NO:161;
k. an HCDR1, an HCDR2, and an HCDR3 as set forth in SEQ ID NO:164, and an LCDR1, an LCDR2, and an LCDR3 as set forth in SEQ ID NO:165;
l. an HCDR1, an HCDR2, and an HCDR3 as set forth in SEQ ID NO:166, and an LCDR1, an LCDR2, and an LCDR3 as set forth in SEQ ID NO:167;
m. an HCDR1, an HCDR2, and an HCDR3 as set forth in SEQ ID NO:168, and an LCDR1, an LCDR2, and an LCDR3 as set forth in SEQ ID NO:169;
n. an HCDR1, an HCDR2, and an HCDR3 as set forth in SEQ ID NO:174, and an LCDR1, an LCDR2, and an LCDR3 as set forth in SEQ ID NO:175;
o. an HCDR1, an HCDR2, and an HCDR3 as set forth in SEQ ID NO:788, and an LCDR1, an LCDR2, and an LCDR3 as set forth in SEQ ID NO:789;
p. an HCDR1, an HCDR2, and an HCDR3 as set forth in SEQ ID NO:186, and an LCDR1, an LCDR2, and an LCDR3 as set forth in SEQ ID NO:187;
q. an HCDR1, an HCDR2, and an HCDR3 as set forth in SEQ ID NO:828, and an LCDR1, an LCDR2, and an LCDR3 as set forth in SEQ ID NO:829;
r. an HCDR1, an HCDR2, and an HCDR3 as set forth in SEQ ID NO:790, and an LCDR1, an LCDR2, and an LCDR3 as set forth in SEQ ID NO:791;
s. an HCDR1, an HCDR2, and an HCDR3 as set forth in SEQ ID NO:188, and an LCDR1, an LCDR2, and an LCDR3 as set forth in SEQ ID NO:189;
t. an HCDR1, an HCDR2, and an HCDR3 as set forth in SEQ ID NO:884, and an LCDR1, an LCDR2, and an LCDR3 as set forth in SEQ ID NO:885;
u. an HCDR1, an HCDR2, and an HCDR3 as set forth in SEQ ID NO:192, and an LCDR1, an LCDR2, and an LCDR3 as set forth in SEQ ID NO:193;
v. an HCDR1, an HCDR2, and an HCDR3 as set forth in SEQ ID NO:194, and an LCDR1, an LCDR2, and an LCDR3 as set forth in SEQ ID NO:195;
w. an HCDR1, an HCDR2, and an HCDR3 as set forth in SEQ ID NO:198, and an LCDR1, an LCDR2, and an LCDR3 as set forth in SEQ ID NO:199;
x. an HCDR1, an HCDR2, and an HCDR3 as set forth in SEQ ID NO:200, and an LCDR1, an LCDR2, and an LCDR3 as set forth in SEQ ID NO:201;
y. an HCDR1, an HCDR2, and an HCDR3 as set forth in SEQ ID NO:202, and an LCDR1, an LCDR2, and an LCDR3 as set forth in SEQ ID NO:203;
z. an HCDR1, an HCDR2, and an HCDR3 as set forth in SEQ ID NO:204, and an LCDR1, an LCDR2, and an LCDR3 as set forth in SEQ ID NO:205;
aa. an HCDR1, an HCDR2, and an HCDR3 as set forth in SEQ ID NO:206, and an LCDR1, an LCDR2, and an LCDR3 as set forth in SEQ ID NO:207;
bb. an HCDR1, an HCDR2, and an HCDR3 as set forth in SEQ ID NO:216, and an LCDR1, an LCDR2, and an LCDR3 as set forth in SEQ ID NO:217;
cc. an HCDR1, an HCDR2, and an HCDR3 as set forth in SEQ ID NO:222, and an LCDR1, an LCDR2, and an LCDR3 as set forth in SEQ ID NO:223;
dd. an HCDR1, an HCDR2, and an HCDR3 as set forth in SEQ ID NO:230, and an LCDR1, an LCDR2, and an LCDR3 as set forth in SEQ ID NO:231;
ee. an HCDR1, an HCDR2, and an HCDR3 as set forth in SEQ ID NO:232, and an LCDR1, an LCDR2, and an LCDR3 as set forth in SEQ ID NO:233;
ff. an HCDR1, an HCDR2, and an HCDR3 as set forth in SEQ ID NO:234, and an LCDR1, an LCDR2, and an LCDR3 as set forth in SEQ ID NO:235;
gg. an HCDR1, an HCDR2, and an HCDR3 as set forth in SEQ ID NO:238, and an LCDR1, an LCDR2, and an LCDR3 as set forth in SEQ ID NO:239;
hh. an HCDR1, an HCDR2, and an HCDR3 as set forth in SEQ ID NO:246, and an LCDR1, an LCDR2, and an LCDR3 as set forth in SEQ ID NO:247;
ii. an HCDR1, an HCDR2, and an HCDR3 as set forth in SEQ ID NO:248, and an LCDR1, an LCDR2, and an LCDR3 as set forth in SEQ ID NO:249;
jj. an HCDR1, an HCDR2, and an HCDR3 as set forth in SEQ ID NO:254, and an LCDR1, an LCDR2, and an LCDR3 as set forth in SEQ ID NO:255;
kk. an HCDR1, an HCDR2, and an HCDR3 as set forth in SEQ ID NO:260, and an LCDR1, an LCDR2, and an LCDR3 as set forth in SEQ ID NO:261;
ll. an HCDR1, an HCDR2, and an HCDR3 as set forth in SEQ ID NO:262, and a LCDR1, an LCDR2, and an LCDR3 as set forth in SEQ ID NO:263; or
mm. an HCDR1, an HCDR2, and an HCDR3 as set forth in SEQ ID NO:264, and an LCDR1, an LCDR2, and an LCDR3 as set forth in SEQ ID NO:265.

In an embodiment, an antibody or antigen binding fragment thereof is provided that binds human interleukin-23 receptor (IL-23R) on a mammalian cell, wherein the antibody or fragment comprises:
a. a VH comprising an HCDR1, an HCDR2, and an HCDR3 having an amino acid sequence of SEQ ID NOs:900, 901, and 902, respectively, and a VL comprising an LCDR1, an LCDR2, and an LCDR3 having an amino acid sequence of SEQ ID NOs:903, 904, and 905, respectively;
b. a VH comprising an HCDR1, an HCDR2, and an HCDR3 having an amino acid sequence of SEQ ID NOs:276, 277, and 278, respectively, and a VL comprising an LCDR1, an LCDR2, and an LCDR3 having an amino acid sequence of SEQ ID NOs:279, 280, and 281, respectively;
c. a VH comprising an HCDR1, an HCDR2, and an HCDR3 having an amino acid sequence of SEQ ID NOs:294, 295, and 296, respectively, and a VL comprising an LCDR1, an LCDR2, and an LCDR3 having an amino acid sequence of SEQ ID NOs:297, 298, and 299, respectively;
d. a VH comprising an HCDR1, an HCDR2, and an HCDR3 having an amino acid sequence of SEQ ID NOs:318, 319, and 320, respectively, and a VL comprising an LCDR1, an LCDR2, and an LCDR3 having an amino acid sequence of SEQ ID NOs:321, 322, and 323, respectively;
e. a VH comprising an HCDR1, an HCDR2, and an HCDR3 having an amino acid sequence of SEQ ID NOs:324, 325, and 326, respectively, and a VL comprising an LCDR1, an LCDR2, and an LCDR3 having an amino acid sequence of SEQ ID NOs:327, 328, and 329, respectively;
f. a VH comprising an HCDR1, an HCDR2, and an HCDR3 having an amino acid sequence of SEQ ID NOs:330, 331, and 332, respectively, and a VL comprising an LCDR1, an LCDR2, and an LCDR3 having an amino acid sequence of SEQ ID NOs:333, 334, and 335, respectively;
g. a VH comprising an HCDR1, an HCDR2, and an HCDR3 having an amino acid sequence of SEQ ID NOs:342, 343, and 344, respectively, and a VL comprising an LCDR1, an LCDR2, and an LCDR3 having an amino acid sequence of SEQ ID NOs:345, 346, and 347, respectively;
h. a VH comprising an HCDR1, an HCDR2, and an HCDR3 having an amino acid sequence of SEQ ID NOs:354, 355, and 356, respectively, and a VL comprising an LCDR1, an LCDR2, and an LCDR3 having an amino acid sequence of SEQ ID NOs:357, 358, and 359, respectively;
i. a VH comprising an HCDR1, an HCDR2, and an HCDR3 having an amino acid sequence of SEQ ID NOs:360, 361, and 362, respectively, and a VL comprising an LCDR1, an LCDR2, and an LCDR3 having an amino acid sequence of SEQ ID NOs:363, 364, and 365, respectively;
j. a VH comprising an HCDR1, an HCDR2, and an HCDR3 having an amino acid sequence of SEQ ID NOs:366, 367, and 368, respectively, and a VL comprising an LCDR1, an LCDR2, and an LCDR3 having an amino acid sequence of SEQ ID NOs:369, 370, and 371, respectively;
k. a VH comprising an HCDR1, an HCDR2, and an HCDR3 having an amino acid sequence of SEQ ID NOs:384, 385, and 386, respectively, and a VL comprising an LCDR1, an LCDR2, and an LCDR3 having an amino acid sequence of SEQ ID NOs:387, 388, and 389, respectively;
l. a VH comprising an HCDR1, an HCDR2, and an HCDR3 having an amino acid sequence of SEQ ID NOs:906, 907, and 908, respectively, and a VL comprising an LCDR1, an LCDR2, and an LCDR3 having an amino acid sequence of SEQ ID NOs:909, 910, and 911, respectively;
m. a VH comprising an HCDR1, an HCDR2, and an HCDR3 having an amino acid sequence of SEQ ID NOs:420, 421, and 422, respectively, and a VL comprising an LCDR1, an LCDR2, and an LCDR3 having an amino acid sequence of SEQ ID NOs:423, 424, and 425, respectively;
n. a VH comprising an HCDR1, an HCDR2, and an HCDR3 having an amino acid sequence of SEQ ID NOs:1026, 1027, and 1028, respectively, and a VL comprising an LCDR1, an LCDR2, and an LCDR3 having an amino acid sequence of SEQ ID NOs:1029, 1030, and 1031, respectively;
o. a VH comprising an HCDR1, an HCDR2, and an HCDR3 having an amino acid sequence of SEQ ID NOs:912, 913, and 914, respectively, and a VL comprising an LCDR1, an LCDR2, and an LCDR3 having an amino acid sequence of SEQ ID NOs:915, 916, and 917, respectively;
p. a VH comprising an HCDR1, an HCDR2, and an HCDR3 having an amino acid sequence of SEQ ID NOs:426, 427, and 428, respectively, and a VL comprising an LCDR1, an LCDR2, and an LCDR3 having an amino acid sequence of SEQ ID NOs:429, 430, and 431, respectively;
q. a VH comprising an HCDR1, an HCDR2, and an HCDR3 having an amino acid sequence of SEQ ID NOs:1194, 1195, and 1196, respectively, and a VL comprising an LCDR1, an LCDR2, and an LCDR3 having an amino acid sequence of SEQ ID NOs:1197, 1198, and 1199, respectively;
r. a VH comprising an HCDR1, an HCDR2, and an HCDR3 having an amino acid sequence of SEQ ID NOs:438, 439, and 440, respectively, and a VL comprising an LCDR1, an LCDR2, and an LCDR3 having an amino acid sequence of SEQ ID NOs:441, 442, and 443, respectively;
s. a VH comprising an HCDR1, an HCDR2, and an HCDR3 having an amino acid sequence of SEQ ID NOs:444, 445, and 446, respectively, and a VL comprising an LCDR1, an LCDR2, and an LCDR3 having an amino acid sequence of SEQ ID NOs:447, 448, and 449, respectively;
t. a VH comprising an HCDR1, an HCDR2, and an HCDR3 having an amino acid sequence of SEQ ID NOs:456, 457, and 458, respectively, and a VL comprising an LCDR1, an LCDR2, and an LCDR3 having an amino acid sequence of SEQ ID NOs:459, 460, and 461, respectively;
u. a VH comprising an HCDR1, an HCDR2, and an HCDR3 having an amino acid sequence of SEQ ID NOs:462, 463, and 464, respectively, and a VL comprising an LCDR1, an LCDR2, and an LCDR3 having an amino acid sequence of SEQ ID NOs:465, 466, and 467, respectively;
v. a VH comprising an HCDR1, an HCDR2, and an HCDR3 having an amino acid sequence of SEQ ID NOs:918, 919, and 920, respectively, and a VL comprising an LCDR1, an LCDR2, and an LCDR3 having an amino acid sequence of SEQ ID NOs:921, 922, and 923, respectively;
w. a VH comprising an HCDR1, an HCDR2, and an HCDR3 having an amino acid sequence of SEQ ID NOs:468, 469, and 470, respectively, and a VL comprising an LCDR1, an LCDR2, and an LCDR3 having an amino acid sequence of SEQ ID NOs:471, 472, and 473, respectively;
x. a VH comprising an HCDR1, an HCDR2, and an HCDR3 having an amino acid sequence of SEQ ID NOs:474, 475, and 476, respectively, and a VL comprising an LCDR1, an LCDR2, and an LCDR3 having an amino acid sequence of SEQ ID NOs:477, 478, and 479, respectively;
y. a VH comprising an HCDR1, an HCDR2, and an HCDR3 having an amino acid sequence of SEQ ID NOs:480, 481, and 482, respectively, and a VL comprising an LCDR1, an LCDR2, and an LCDR3 having an amino acid sequence of SEQ ID NOs:483, 484, and 485, respectively;
z. a VH comprising an HCDR1, an HCDR2, and an HCDR3 having an amino acid sequence of SEQ ID NOs:510, 511, and 512, respectively, and a VL comprising an LCDR1, an LCDR2, and an LCDR3 having an amino acid sequence of SEQ ID NOs:513, 514, and 515, respectively;
aa. a VH comprising an HCDR1, an HCDR2, and an HCDR3 having an amino acid sequence of SEQ ID NOs:528, 529, and 530, respectively, and a VL comprising an LCDR1, an LCDR2, and an LCDR3 having an amino acid sequence of SEQ ID NOs:531, 532, and 533, respectively;
bb. a VH comprising an HCDR1, an HCDR2, and an HCDR3 having an amino acid sequence of SEQ ID NOs:936, 937, and 938, respectively, and a VL comprising an LCDR1, an LCDR2, and an LCDR3 having an amino acid sequence of SEQ ID NOs:939, 940, and 941, respectively;
cc. a VH comprising an HCDR1, an HCDR2, and an HCDR3 having an amino acid sequence of SEQ ID NOs:552, 553, and 554, respectively, and a VL comprising an LCDR1, an LCDR2, and an LCDR3 having an amino acid sequence of SEQ ID NOs:555, 556, and 557, respectively;
dd. a VH comprising an HCDR1, an HCDR2, and an HCDR3 having an amino acid sequence of SEQ ID NOs:558, 559, and 560, respectively, and a VL comprising an LCDR1, an LCDR2, and an LCDR3 having an amino acid sequence of SEQ ID NOs:561, 562, and 563, respectively;
ee. a VH comprising an HCDR1, an HCDR2, and an HCDR3 having an amino acid sequence of SEQ ID NOs:564, 565, and 566, respectively, and a VL comprising an LCDR1, an LCDR2, and an LCDR3 having an amino acid sequence of SEQ ID NOs:567, 568, and 569, respectively;
ff. a VH comprising an HCDR1, an HCDR2, and an HCDR3 having an amino acid sequence of SEQ ID NOs:570, 571, and 572, respectively, and a VL comprising an LCDR1, an LCDR2, and an LCDR3 having an amino acid sequence of SEQ ID NOs:573, 574, and 575, respectively;
gg. a VH comprising an HCDR1, an HCDR2, and an HCDR3 having an amino acid sequence of SEQ ID NOs:576, 577, and 578, respectively, and a VL comprising an LCDR1, an LCDR2, and an LCDR3 having an amino acid sequence of SEQ ID NOs:579, 580, and 581, respectively;
hh. a VH comprising an HCDR1, an HCDR2, and an HCDR3 having an amino acid sequence of SEQ ID NOs:600, 601, and 602, respectively, and a VL comprising an LCDR1, an LCDR2, and an LCDR3 having an amino acid sequence of SEQ ID NOs:603, 604, and 605, respectively;
ii. a VH comprising an HCDR1, an HCDR2, and an HCDR3 having an amino acid sequence of SEQ ID NOs:606, 607, and 608, respectively, and a VL comprising an LCDR1, an LCDR2, and an LCDR3 having an amino acid sequence of SEQ ID NOs:609, 610, and 611, respectively;
jj. a VH comprising an HCDR1, an HCDR2, and an HCDR3 having an amino acid sequence of SEQ ID NOs:624, 625, and 626, respectively, and a VL comprising an LCDR1, an LCDR2, and an LCDR3 having an amino acid sequence of SEQ ID NOs:627, 628, and 629, respectively;
kk. a VH comprising an HCDR1, an HCDR2, and an HCDR3 having an amino acid sequence of SEQ ID NOs:642, 643, and 644, respectively, and a VL comprising an LCDR1, an LCDR2, and an LCDR3 having an amino acid sequence of SEQ ID NOs:645, 646, and 647, respectively;
ll. a VH comprising an HCDR1, an HCDR2, and an HCDR3 having an amino acid sequence of SEQ ID NOs:648, 649, and 650, respectively, and a VL comprising an LCDR1, an LCDR2, and an LCDR3 having an amino acid sequence of SEQ ID NOs:651, 652, and 653, respectively; or
mm. a VH comprising an HCDR1, an HCDR2, and an HCDR3 having an amino acid sequence of SEQ ID NOs:654, 655, and 656, respectively, and a VL comprising an LCDR1, an LCDR2, and an LCDR3 having an amino acid sequence of SEQ ID NOs:657, 658, and 659, respectively.

In an embodiment, an antibody or antigen binding fragment thereof is provided that binds human interleukin-23 receptor (IL-23R) on a mammalian cell, wherein the antibody or fragment comprises:
a. a VH comprising the amino acid sequence of SEQ ID NO:786, and a VL comprising the amino acid sequence of SEQ ID NO:787;
b. a VH comprising the amino acid sequence of SEQ ID NO:138, and a VL comprising the amino acid sequence of SEQ ID NO:139;
c. a VH comprising the amino acid sequence of SEQ ID NO:144, and a VL comprising the amino acid sequence of SEQ ID NO:145;
d. a VH comprising the amino acid sequence of SEQ ID NO:152, and a VL comprising the amino acid sequence of SEQ ID NO:153;
e. a VH comprising the amino acid sequence of SEQ ID NO:154, and a VL comprising the amino acid sequence of SEQ ID NO:155;
f. a VH comprising the amino acid sequence of SEQ ID NO:156, and a VL comprising the amino acid sequence of SEQ ID NO:157;
g. a VH comprising the amino acid sequence of SEQ ID NO:160, and a VL comprising the amino acid sequence of SEQ ID NO:161;
h. a VH comprising the amino acid sequence of SEQ ID NO:164, and a VL comprising the amino acid sequence of SEQ ID NO:165;
i. a VH comprising the amino acid sequence of SEQ ID NO:166, and a VL comprising the amino acid sequence of SEQ ID NO:167;
j. a VH comprising the amino acid sequence of SEQ ID NO:168, and a VL comprising the amino acid sequence of SEQ ID NO:169;
k. a VH comprising the amino acid sequence of SEQ ID NO:174, and a VL comprising the amino acid sequence of SEQ ID NO:175;
l. a VH comprising the amino acid sequence of SEQ ID NO:788, and a VL comprising the amino acid sequence of SEQ ID NO:789;
m. a VH comprising the amino acid sequence of SEQ ID NO:186, and a VL comprising the amino acid sequence of SEQ ID NO:187;
n. a VH comprising the amino acid sequence of SEQ ID NO:828, and a VL comprising the amino acid sequence of SEQ ID NO:829;
o. a VH comprising the amino acid sequence of SEQ ID NO:790, and a VL comprising the amino acid sequence of SEQ ID NO:791;
p. a VH comprising the amino acid sequence of SEQ ID NO:188, and a VL comprising the amino acid sequence of SEQ ID NO:189;
q. a VH comprising the amino acid sequence of SEQ ID NO:884, and a VL comprising the amino acid sequence of SEQ ID NO:885;
r. a VH comprising the amino acid sequence of SEQ ID NO:192, and a VL comprising the amino acid sequence of SEQ ID NO:193;
s. a VH comprising the amino acid sequence of SEQ ID NO:194, and a VL comprising the amino acid sequence of SEQ ID NO:195;
t. a VH comprising the amino acid sequence of SEQ ID NO:198, and a VL comprising the amino acid sequence of SEQ ID NO:199;
u. a VH comprising the amino acid sequence of SEQ ID NO:200, and a VL comprising the amino acid sequence of SEQ ID NO:201;
v. a VH comprising the amino acid sequence of SEQ ID NO:792, and a VL comprising the amino acid sequence of SEQ ID NO:793;
w. a VH comprising the amino acid sequence of SEQ ID NO:202, and a VL comprising the amino acid sequence of SEQ ID NO:203;
x. a VH comprising the amino acid sequence of SEQ ID NO:204, and a VL comprising the amino acid sequence of SEQ ID NO:205;
y. a VH comprising the amino acid sequence of SEQ ID NO:206, and a VL comprising the amino acid sequence of SEQ ID NO:207;
z. a VH comprising the amino acid sequence of SEQ ID NO:216, and a VL comprising the amino acid sequence of SEQ ID NO:217;
aa. a VH comprising the amino acid sequence of SEQ ID NO:222, and a VL comprising the amino acid sequence of SEQ ID NO:223;
bb. a VH comprising the amino acid sequence of SEQ ID NO:798, and a VL comprising the amino acid sequence of SEQ ID NO:799;
cc. a VH comprising the amino acid sequence of SEQ ID NO:230, and a VL comprising the amino acid sequence of SEQ ID NO:231;
dd. a VH comprising the amino acid sequence of SEQ ID NO:232, and a VL comprising the amino acid sequence of SEQ ID NO:233;
ee. a VH comprising the amino acid sequence of SEQ ID NO:234, and a VL comprising the amino acid sequence of SEQ ID NO:235;
ff. a VH comprising the amino acid sequence of SEQ ID NO:236, and a VL comprising the amino acid sequence of SEQ ID NO:237;
gg. a VH comprising the amino acid sequence of SEQ ID NO:238, and a VL comprising the amino acid sequence of SEQ ID NO:239;
hh. a VH comprising the amino acid sequence of SEQ ID NO:246, and a VL comprising the amino acid sequence of SEQ ID NO:247;
ii. a VH comprising the amino acid sequence of SEQ ID NO:248, and a VL comprising the amino acid sequence of SEQ ID NO:249;
jj. a VH comprising the amino acid sequence of SEQ ID NO:254, and a VL comprising the amino acid sequence of SEQ ID NO:255;
kk. a VH comprising the amino acid sequence of SEQ ID NO:260, and a VL comprising the amino acid sequence of SEQ ID NO:261;
ll. a VH comprising the amino acid sequence of SEQ ID NO:262, and a VL comprising the amino acid sequence of SEQ ID NO:263; or
mm. a VH comprising the amino acid sequence of SEQ ID NO:264, and a VL comprising the amino acid sequence of SEQ ID NO:265.

In some embodiments, antibody that binds human interleukin-23 receptor (IL-23R) on a mammalian cell is selected from antibody I23RB1, I23RB3, I23RB4, I23RB7 and I23RB76. In an embodiment, the mammalian cell is a human cell or a rat cell. In an embodiment, the cell is an immune cell. In an embodiment, the cell is a primary immune cell. In an embodiment, the cell is a human primary immune cell or a rat primary immune cell. In an embodiment, the cell is a human T cell or a rat T cell. In embodiments, the cell(s) may be or may include include αβ T cells, including CD4+ and CD8+ T cells, γδ T cells, natural killer (NK) cells, innate lymphoid cells (ILCs), or monocyte-derived macrophages. In embodiments, the cell(s) may be or may include include αβ T cells, including CD4+ and CD8+ T cells, or γδ T cells. In some embodiments, the cell is a natural killer (NK) cell. In some embodiments, the cell is an innate lymphoid cell (ILC). In some embodiments, the cell is a monocyte-derived macrophage.

In an embodiment, monoclonal antibody or active fragment thereof is provided that binds human interleukin-23 receptor (IL-23R) on a mammalian cell of claim 1, wherein the antibody or fragment comprises:
a. an HCDR1, and HCDR2 and an HCDR3 as set forth in SEQ ID NO:786, and an LCDR1, LCDR2 and LCDR3 set forth in SEQ ID NO:787;
b. an HCDR1, and HCDR2 and an HCDR3 as set forth in SEQ ID NO:790, and an LCDR1, LCDR2 and LCDR3 set forth in SEQ ID NO:791;
c. an HCDR1, and HCDR2 and an HCDR3 as set forth in SEQ ID NO:792, and an LCDR1, LCDR2 and LCDR3 set forth in SEQ ID NO:793;
d. an HCDR1, and HCDR2 and an HCDR3 as set forth in SEQ ID NO:798, and an LCDR1, LCDR2 and LCDR3 set forth in SEQ ID NO:799; or
e. an HCDR1, and HCDR2 and an HCDR3 as set forth in SEQ ID NO:236, and an LCDR1, LCDR2 and LCDR3 set forth in SEQ ID NO:237.

In an embodiment, antibody or antigen binding fragment thereof is provided that binds human interleukin-23 receptor (IL-23R) on a mammalian cell, wherein the antibody or fragment comprises:
a. a VH comprising an HCDR1, and HCDR2 and an HCDR3 having an amino acid sequence of SEQ ID NOs:900, 901, and 902, respectively and a VL comprising an LCDR1, an LCDR2, and an LCDR3 having an amino acid sequence of SEQ ID NOs:903, 904, and 905, respectively;
b. a VH comprising an HCDR1, and HCDR2 and an HCDR3 having an amino acid sequence of SEQ ID NOs:912, 913, and 914, respectively and a VL comprising an LCDR1, an LCDR2, and an LCDR3 having an amino acid sequence of SEQ ID NOs:915, 916, and 917, respectively;
c. a VH comprising an HCDR1, and HCDR2 and an HCDR3 having an amino acid sequence of SEQ ID NOs:918, 919, and 920, respectively and a VL comprising an LCDR1, an LCDR2, and an LCDR3 having an amino acid sequence of SEQ ID NOs:921, 922, and 923, respectively;
d. a VH comprising an HCDR1, and HCDR2 and an HCDR3 having an amino acid sequence of SEQ ID NOs:936, 937, and 938, respectively and a VL comprising an LCDR1, an LCDR2, and an LCDR3 having an amino acid sequence of SEQ ID NOs:939, 940, and 941, respectively; or
e. a VH comprising an HCDR1, and HCDR2 and an HCDR3 having an amino acid sequence of SEQ ID NOs:570, 571, and 572, respectively and a VL comprising an LCDR1, an LCDR2, and an LCDR3 having an amino acid sequence of SEQ ID NOs:573, 574, and 575, respectively.

In an embodiment, antibody or antigen binding fragment thereof is provided that binds human interleukin-23 receptor (IL-23R) on a mammalian cell, wherein the antibody or fragment comprises:
a. a VH comprising the amino acid sequence of SEQ ID NO:786, and a VL comprising the amino acid sequence of SEQ ID NO:787;
b. a VH comprising the amino acid sequence of SEQ ID NO:790, and a VL comprising the amino acid sequence of SEQ ID NO:791;
c. a VH comprising the amino acid sequence of SEQ ID NO:792, and a VL comprising the amino acid sequence of SEQ ID NO:793;
d. a VH comprising the amino acid sequence of SEQ ID NO:798, and a VL comprising the amino acid sequence of SEQ ID NO:799; or
e. a VH comprising the amino acid sequence of SEQ ID NO:236, and a VL comprising the amino acid sequence of SEQ ID NO:237.

In some embodiments, antibody that binds interleukin-23 receptor (IL-23R) on a mammalian cell is selected from I23RB4, I23RB7 and I23RB76. In an embodiment, the mammalian cell is a human cell or a rat cell. In an embodiment, the cell is an immune cell. In an embodiment, the cell is a primary immune cell. In an embodiment, the cell is a human primary immune cell or a rat primary immune cell. In an embodiment, the cell is a human T cell or a rat T cell. In embodiments, the cell(s) may be or may include include αβ T cells, including CD4+ and CD8+ T cells, γδ T cells, natural killer (NK) cells, innate lymphoid cells (ILCs), or monocyte-derived macrophages. In embodiments, the cell(s) may be or may include include αβ T cells, including CD4+ and CD8+ T cells, or γδ T cells. In some embodiments, the cell is a natural killer (NK) cell. In some embodiments, the cell is an innate lymphoid cell (ILC). In some embodiments, the cell is a monocyte-derived macrophage.

In an embodiment thereof, antibody or antigen binding fragment thereof is provided that binds human interleukin-23 receptor (IL-23R) on the surface of cells including primary human immune cells, wherein the antibody or fragment comprises:
a. a VH comprising an HCDR1, and HCDR2 and an HCDR3 having an amino acid sequence of SEQ ID NOs:918, 919, and 920, respectively and a VL comprising an LCDR1, an LCDR2, and an LCDR3 having an amino acid sequence of SEQ ID NOs:921, 922, and 923, respectively;
b. a VH comprising an HCDR1, and HCDR2 and an HCDR3 having an amino acid sequence of SEQ ID NOs:936, 937, and 938, respectively and a VL comprising an LCDR1, an LCDR2, and an LCDR3 having an amino acid sequence of SEQ ID NOs:939, 940, and 941, respectively; or
c. a VH comprising an HCDR1, and HCDR2 and an HCDR3 having an amino acid sequence of SEQ ID NOs:570, 571, and 572, respectively and a VL comprising an LCDR1, an LCDR2, and an LCDR3 having an amino acid sequence of SEQ ID NOs:573, 574, and 575, respectively.

In an embodiment, antibody or antigen binding fragment thereof is provided that binds human interleukin-23 receptor (IL-23R) on a mammalian cell, wherein the antibody or fragment comprises:
a. a VH comprising the amino acid sequence of SEQ ID NO:792, and a VL comprising the amino acid sequence of SEQ ID NO:793;
b. a VH comprising the amino acid sequence of SEQ ID NO:798, and a VL comprising the amino acid sequence of SEQ ID NO:799; or
c. a VH comprising the amino acid sequence of SEQ ID NO:236, and a VL comprising the amino acid sequence of SEQ ID NO:237.

In another embodiment, antibody and antigen binding fragments thereof are provided herein wherein the antibody (and binding fragment) recognizes and/or binds both human and rat IL-23R. In embodiments, the antibody and fragment recognizes and binds both rat and human IL-23R extracellular domain (ECD). In some embodiments, antibody is selected from I23RB42, I23RB85, I23RB157, I23RB7, I23RB153, I23RB45, I23RB318, I23RB30 and 123RB56

In some embodiments, isolated monoclonal antibody or antigen binding fragment thereof is provided which recognizes human and rat IL-23R, wherein the antibody or fragment comprises:
a. an HCDR1, and HCDR2 and an HCDR3 as set forth in SEQ ID NO:202, and an LCDR1, LCDR2 and LCDR3 set forth in SEQ ID NO:203;
b. an HCDR1, and HCDR2 and an HCDR3 as set forth in SEQ ID NO:252, and an LCDR1, LCDR2 and LCDR3 set forth in SEQ ID NO:253;
c. an HCDR1, and HCDR2 and an HCDR3 as set forth in SEQ ID NO:156, and an LCDR1, LCDR2 and LCDR3 set forth in SEQ ID NO:157;
d. an HCDR1, and HCDR2 and an HCDR3 as set forth in SEQ ID NO:798, and an LCDR1, LCDR2 and LCDR3 set forth in SEQ ID NO:799;
e. an HCDR1, and HCDR2 and an HCDR3 as set forth in SEQ ID NO:150, and an LCDR1, LCDR2 and LCDR3 set forth in SEQ ID NO:151;
f. an HCDR1, and HCDR2 and an HCDR3 as set forth in SEQ ID NO:204, and an LCDR1, LCDR2 and LCDR3 set forth in SEQ ID NO:205;
g. an HCDR1, and HCDR2 and an HCDR3 as set forth in SEQ ID NO:884, and an LCDR1, LCDR2 and LCDR3 set forth in SEQ ID NO:885;
h. an HCDR1, and HCDR2 and an HCDR3 as set forth in SEQ ID NO:188, and an LCDR1, LCDR2 and LCDR3 set forth in SEQ ID NO:189; or
i. an HCDR1, and HCDR2 and an HCDR3 as set forth in SEQ ID NO:212, and an LCDR1, LCDR2 and LCDR3 set forth in SEQ ID NO:213.

In embodiments, antibody or antigen binding fragment thereof is provided which recognizes human and rat IL-23R, wherein the antibody or fragment comprises:
a. a VH comprising an HCDR1, and HCDR2 and an HCDR3 having an amino acid sequence of SEQ ID NOs:468, 469, and 470, respectively and a VL comprising an LCDR1, an LCDR2, and an LCDR3 having an amino acid sequence of SEQ ID NOs:471, 472, and 473, respectively;
b. a VH comprising an HCDR1, and HCDR2 and an HCDR3 having an amino acid sequence of SEQ ID NOs:618, 619, and 620, respectively and a VL comprising an LCDR1, an LCDR2, and an LCDR3 having an amino acid sequence of SEQ ID NOs:621, 622, and 623,
c. a VH comprising an HCDR1, and HCDR2 and an HCDR3 having an amino acid sequence of SEQ ID NOs:330, 331, and 332, respectively and a VL comprising an LCDR1, an LCDR2, and an LCDR3 having an amino acid sequence of SEQ ID NOs:333, 334, and 335, respectively;
d. a VH comprising an HCDR1, and HCDR2 and an HCDR3 having an amino acid sequence of SEQ ID NOs:936,937, and 938, respectively and a VL comprising an LCDR1, an LCDR2, and an LCDR3 having an amino acid sequence of SEQ ID NOs:939, 940, and 941, respectively;
e. a VH comprising an HCDR1, and HCDR2 and an HCDR3 having an amino acid sequence of SEQ ID NOs:312, 313, and 314, respectively and a VL comprising an LCDR1, an LCDR2, and an LCDR3 having an amino acid sequence of SEQ ID NOs:315, 316, and 317, respectively;
f. a VH comprising an HCDR1, and HCDR2 and an HCDR3 having an amino acid sequence of SEQ ID NOs:474, 475, and 476, respectively and a VL comprising an LCDR1, an LCDR2, and an LCDR3 having an amino acid sequence of SEQ ID NOs:477, 478, and 479, respectively;
g. a VH comprising an HCDR1, and HCDR2 and an HCDR3 having an amino acid sequence of SEQ ID NOs:1194, 1195, and 1196, respectively and a VL comprising an LCDR1, an LCDR2, and an LCDR3 having an amino acid sequence of SEQ ID NOs:1197, 1198, and 1199, respectively;
h. a VH comprising an HCDR1, and HCDR2 and an HCDR3 having an amino acid sequence of SEQ ID NOs:426, 427, and 428, respectively and a VL comprising an LCDR1, an LCDR2, and an LCDR3 having an amino acid sequence of SEQ ID NOs:429, 430, and 431, respectively; or
i. a VH comprising an HCDR1, and HCDR2 and an HCDR3 having an amino acid sequence of SEQ ID NOs:498, 499, and 500, respectively and a VL comprising an LCDR1, an LCDR2, and an LCDR3 having an amino acid sequence of SEQ ID NOs:501, 502, and 503, respectively.

In some embodiments, antibody or antigen binding fragment thereof is provided which recognizes human and rat IL-23R, wherein the antibody or fragment comprises:
a. a VH comprising the amino acid sequence of SEQ ID NO:202, and a VL comprising the amino acid sequence of SEQ ID NO:203;
b. a VH comprising the amino acid sequence of SEQ ID NO:252, and a VL comprising the amino acid sequence of SEQ ID NO:253;
c. a VH comprising the amino acid sequence of SEQ ID NO:156, and a VL comprising the amino acid sequence of SEQ ID NO:157;
d. a VH comprising the amino acid sequence of SEQ ID NO:798, and a VL comprising the amino acid sequence of SEQ ID NO:799;
e. a VH comprising the amino acid sequence of SEQ ID NO:150, and a VL comprising the amino acid sequence of SEQ ID NO:151;
f. a VH comprising the amino acid sequence of SEQ ID NO:204, and a VL comprising the amino acid sequence of SEQ ID NO:205;
g. a VH comprising the amino acid sequence of SEQ ID NO:884, and a VL comprising the amino acid sequence of SEQ ID NO:885;
h. a VH comprising the amino acid sequence of SEQ ID NO:188, and a VL comprising the amino acid sequence of SEQ ID NO:189; or
i. a VH comprising the amino acid sequence of SEQ ID NO:212, and a VL comprising the amino acid sequence of SEQ ID NO:213.

In other embodiments, antibodies are provided which share a common light chain sequence. In some embodiments, the antibodies share a VL amino acid sequence. In come embodiments, the antibodies share a LC amino acid sequence. In embodiments, the antibodies share light chain CDR1, CDR2 and CDR3 amino acid sequences.

In a first group (Group 1), the following antibodies share identical VL, LC and light chain CDR sequences: I23RB1, RB2, RB3, RB4, RB8, RB9, RB268, RB270, RB291. In accordance with one aspect ooof this embodiment is provided antibody or antigen binding fragment thereof that binds IL-23R comprising a VL comprising CDR sequences of an LCDR1 SEQ ID NO:1252, an LCDR2 SEQ ID NO:1253, and an LCDR3 SEQ ID NO:1254 and a VH comprising CDR sequences selected from:
a. an HCDR1 SEQ ID NO:900, an HCDR2 SEQ ID NO:901 and an HCDR3 SEQ ID NO:902;
b. an HCDR1 SEQ ID NO:906, an HCDR2 SEQ ID NO:907, and an HCDR3 SEQ ID NO:908;
c. an HCDR1 SEQ ID NO:912, an HCDR2 SEQ ID NO:913, and an HCDR3 SEQ ID NO:914;
d. an HCDR1 SEQ ID NO:918, an HCDR2 SEQ ID NO:919, and an HCDR3 SEQ ID NO:920;
e. an HCDR1 SEQ ID NO:942, an HCDR2 SEQ ID NO:943, and an HCDR3 SEQ ID NO:944;
f. an HCDR1 SEQ ID NO:948, an HCDR2 SEQ ID NO:949, and an HCDR3 SEQ ID NO:950;
g. an HCDR1 SEQ ID NO: 1014, an HCDR2 SEQ ID NO: 1015, and an HCDR3 SEQ ID NO:1016;
h. an HCDR1 SEQ ID NO: 1026, an HCDR2 SEQ ID NO: 1027, and an HCDR3 SEQ ID NO:1028; and
i. an HCDR1 SEQ ID NO:1152, an HCDR2 SEQ ID NO:1153, and an HCDR3 SEQ ID NO:1154.

In an embodiment antibody or antigen binding fragment thereof is provided comprising a VL comprising CDR sequences of an LCDR1 SEQ ID NO:1252, an LCDR2 SEQ ID NO:1253, and an LCDR3 SEQ ID NO: 1254 or having the amino acid sequence SEQ ID NO:1247 and a VH sequence selected from SEQ ID NO:786, 788, 790, 792, 800, 802, 824, 828 and 870.

In a second group (Group 2), the following antibodies share identical VL, LC and light chain CDR sequences: I23RB5, I23RB266, I23RB271, I23RB273, I23RB274, I23RB275, I23RB276, I23RB277, I23RB279, I23RB281, I23RB282, I23RB284, I23RB285, I23RB286, I23RB289, I23RB290 and I23RB292. In accordance with one aspect of this embodiment is provided antibody or antigen binding fragment thereof that binds IL-23R comprising a VL comprising CDR sequences of an LCDR1 SEQ ID NO: 1255, an LCDR2 SEQ ID NO:1256, and an LCDR3 SEQ ID NO:1257 and a VH comprising CDR sequences selected from:
a. an HCDR1 SEQ ID NO:924, an HCDR2 SEQ ID NO:925, and an HCDR3 SEQ ID NO:926;
b. an HCDR1 SEQ ID NO: 1002, an HCDR2 SEQ ID NO: 1003, and an HCDR3 SEQ ID NO: 1004;
c. an HCDR1 SEQ ID NO:1032, an HCDR2 SEQ ID NO:1033, and an HCDR3 SEQ ID NO: 1034;
d. an HCDR1 SEQ ID NO: 1044, an HCDR2 SEQ ID NO: 1045, and an HCDR3 SEQ ID NO: 1046;
e. an HCDR1 SEQ ID NO:1050, an HCDR2 SEQ ID NO:1051, and an HCDR3 SEQ ID NO: 1052;
f. an HCDR1 SEQ ID NO: 1056, an HCDR2 SEQ ID NO: 1057, and an HCDR3 SEQ ID NO: 1058;
g. an HCDR1 SEQ ID NO: 1062, an HCDR2 SEQ ID NO: 1063, and an HCDR3 SEQ ID NO: 1064;
h. an HCDR1 SEQ ID NO: 1068, an HCDR2 SEQ ID NO: 1069, and an HCDR3 SEQ ID NO: 1070;
i. an HCDR1 SEQ ID NO:1080, an HCDR2 SEQ ID NO:1081, and an HCDR3 SEQ ID NO: 1082;
j. an HCDR1 SEQ ID NO:1092, an HCDR2 SEQ ID NO:1093, and an HCDR3 SEQ ID NO: 1094;
k. an HCDR1 SEQ ID NO: 1098, an HCDR2 SEQ ID NO: 1099, and an HCDR3 SEQ ID NO: 1100;
l. an HCDR1 SEQ ID NO:1110, an HCDR2 SEQ ID NO:1111, and an HCDR3 SEQ ID NO:1112;
m. an HCDR1 SEQ ID NO:1122, an HCDR2 SEQ ID NO:1123, and an HCDR3 SEQ ID NO:1124;
n. an HCDR1 SEQ ID NO:1140, an HCDR2 SEQ ID NO:1141, and an HCDR3 SEQ ID NO:1142;
o. an HCDR1 SEQ ID NO: 1146, an HCDR2 SEQ ID NO: 1147, and an HCDR3 SEQ ID NO:1148; and
p. an HCDR1 SEQ ID NO: 1158, an HCDR2 SEQ ID NO: 1159, and an HCDR3 SEQ ID NO:1160.

In some embodiments, antibody or antigen binding fragment thereof is provided comprising a VL comprising CDR sequences of an LCDR1 SEQ ID NO:1255, an LCDR2 SEQ ID NO:1256, and an LCDR3 SEQ ID NO: 1257 or having the amino acid sequence SEQ ID NO:1248 and a VH sequence selected from SEQ ID NO:794, 820, 830, 834, 836, 838, 840, 842, 846, 850, 852, 856, 860, 866, 868 and 872.

In a third group (Group 3), the following antibodies share identical VL, LC and light chain CDR sequences: I23RB6 and I23RB10. In accordance with one aspect of this embodiment is provided antibody or antigen binding fragment thereof that binds IL-23R comprising a VL comprising CDR sequences of an LCDR1 SEQ ID NO:1258, an LCDR2 SEQ ID NO:1259, and an LCDR3 SEQ ID NO: 1260 and a VH comprising CDR sequences selected from:
a. an HCDR1 SEQ ID NO:930, an HCDR2 SEQ ID NO:931, and an HCDR3 SEQ ID NO:932; and
b. an HCDR1 SEQ ID NO:954, an HCDR2 SEQ ID NO:955, and an HCDR3 SEQ ID NO:956.

In an embodiment, antibody or antigen binding fragment thereof is provided comprising a VL comprising CDR sequences of an LCDR1 SEQ ID NO:1258, an LCDR2 SEQ ID NO:1259, and an LCDR3 SEQ ID NO: 1260 or having the amino acid sequence SEQ ID NO:1249 and a VH sequence selected from SEQ ID NO:796 and 804.

In a fourth group (Group 2), the following antibodies share identical VL, LC and light chain CDR sequences: I23RB259 and I23RB316. In accordance with one aspect of this embodiment is provided antibody or antigen binding fragment thereof that binds IL-23R comprising a VL comprising CDR sequences of an LCDR1 SEQ ID NO:1261, an LCDR2 SEQ ID NO:1262, and an LCDR3 SEQ ID NO:1263 and a VH comprising CDR sequences selected from:
a. an HCDR1 SEQ ID NO:960, an HCDR2 SEQ ID NO:961, and an HCDR3 SEQ ID NO:962; and
b. an HCDR1 SEQ ID NO:1182, an HCDR2 SEQ ID NO:1183, and an HCDR3 SEQ ID NO:1184.

In an embodiment, antibody or antigen binding fragment thereof is provided comprising a VL comprising CDR sequences of an LCDR1 SEQ ID NO:1261, an LCDR2 SEQ ID NO:1262, and an LCDR3 SEQ ID NO: 1263 or having the amino acid sequence SEQ ID NO:1250 and a VH sequence selected from SEQ ID NO:806 and 880.

In a fifth group (Group 5), the following antibodies share identical VL, LC and light chain CDR sequences: I23RB267, I23RB3269, I23RB272, I23RB278, I23RB280, I23RB283, I23RB287 and I23RB288. In accordance with one aspect of this embodiment is provided antibody or antigen binding fragment thereof that binds IL-23R comprising a VL comprising CDR sequences of an LCDR1 SEQ ID NO:1264, an LCDR2 SEQ ID NO:1265, and an LCDR3 SEQ ID NO: 1266 and a VH comprising CDR sequences selected from:
a. an HCDR1 SEQ ID NO:1008, an HCDR2 SEQ ID NO:1009, and an HCDR3 SEQ ID NO:1010;
b. an HCDR1 SEQ ID NO: 1020, an HCDR2 SEQ ID NO:1021, and an HCDR3 SEQ ID NO:1022;
c. an HCDR1 SEQ ID NO:1038, an HCDR2 SEQ ID NO:1039, and an HCDR3 SEQ ID NO: 1040;
d. an HCDR1 SEQ ID NO: 1074, an HCDR2 SEQ ID NO: 1075, and an HCDR3 SEQ ID NO: 1076;
e. an HCDR1 SEQ ID NO:1086, an HCDR2 SEQ ID NO:1087, and an HCDR3 SEQ ID NO: 1088;
f. an HCDR1 SEQ ID NO: 1104, an HCDR2 SEQ ID NO: 1105, and an HCDR3 SEQ ID NO: 1106;
g. an HCDR1 SEQ ID NO: 1128, an HCDR2 SEQ ID NO: 1129, and an HCDR3 SEQ ID NO:1130; and
h. an HCDR1 SEQ ID NO:1134, an HCDR2 SEQ ID NO:1135, and an HCDR3 SEQ ID NO: 1136.

In an embodiment, antibody or antigen binding fragment thereof is provided comprising a VL comprising CDR sequences of an LCDR1 SEQ ID NO:1264, an LCDR2 SEQ ID NO:1265, and an LCDR3 SEQ ID NO: 1266 or having the amino acid sequence SEQ ID NO:1251 and a VH sequence selected from SEQ ID NO: 806 and 880 822, 826, 832, 844, 848, 854, 862 and 864.

In a particular embodiment of the invention, a VHH antibody (also denoted as heavy chain domain antibody or as a nanobody) are provided, including antibodies or fragments thereof which consist of heavy chain sequence only. In an embodiment, herein is provided VHH antibody or nanobody that binds IL-23R comprising a VH sequence selected from SEQ ID NO:786, 788, 790, 792, 800, 802, 824, 828, 870, 794, 820, 830, 834, 836, 838, 840, 842, 846, 850, 852, 856, 860, 866, 868, 872, 796, 804, 806, 880, 822, 826, 832, 844, 848, 854, 862 and 864.

IL-23R binding antibodies are provided herein. In some embodiments, antibody or antigen binding fragment thereof is provided that binds IL-23R comprising:
a. an HCDR1, an HCDR2, and an HCDR3 as set forth in SEQ ID NO:138, and an LCDR1, an LCDR2, and an LCDR3 as set forth in SEQ ID NO:139;
b. an HCDR1, an HCDR2, and an HCDR3 as set forth in SEQ ID NO:142, and an LCDR1, an LCDR2, and an LCDR3 as set forth in SEQ ID NO:143;
c. an HCDR1, an HCDR2, and an HCDR3 as set forth in SEQ ID NO:144, and an LCDR1, an LCDR2, and an LCDR3 as set forth in SEQ ID NO:145;
d. an HCDR1, an HCDR2, and an HCDR3 as set forth in SEQ ID NO:148, and an LCDR1, an LCDR2, and an LCDR3 as set forth in SEQ ID NO:149;
e. an HCDR1, an HCDR2, and an HCDR3 as set forth in SEQ ID NO:150, and an LCDR1, an LCDR2, and an LCDR3 as set forth in SEQ ID NO:151;
f. an HCDR1, an HCDR2, and an HCDR3 as set forth in SEQ ID NO:152, and an LCDR1, an LCDR2, and an LCDR3 as set forth in SEQ ID NO:153;
g. an HCDR1, an HCDR2, and an HCDR3 as set forth in SEQ ID NO:154, and an LCDR1, an LCDR2, and an LCDR3 as set forth in SEQ ID NO:155;
h. an HCDR1, an HCDR2, and an HCDR3 as set forth in SEQ ID NO:156, and an LCDR1, an LCDR2, and an LCDR3 as set forth in SEQ ID NO:157;
i. an HCDR1, an HCDR2, and an HCDR3 as set forth in SEQ ID NO:158, and an LCDR1, an LCDR2, and an LCDR3 as set forth in SEQ ID NO:159;
j. an HCDR1, an HCDR2, and an HCDR3 as set forth in SEQ ID NO:160, and an LCDR1, an LCDR2, and an LCDR3 as set forth in SEQ ID NO:161;
k. an HCDR1, an HCDR2, and an HCDR3 as set forth in SEQ ID NO:162, and an LCDR1, an LCDR2, and an LCDR3 as set forth in SEQ ID NO:163;
l. an HCDR1, an HCDR2, and an HCDR3 as set forth in SEQ ID NO:164, and an LCDR1, an LCDR2, and an LCDR3 as set forth in SEQ ID NO:165;
m. an HCDR1, an HCDR2, and an HCDR3 as set forth in SEQ ID NO:166, and an LCDR1, an LCDR2, and an LCDR3 as set forth in SEQ ID NO:167;
n. an HCDR1, an HCDR2, and an HCDR3 as set forth in SEQ ID NO:168, and an LCDR1, an LCDR2, and an LCDR3 as set forth in SEQ ID NO:169;
o. an HCDR1, an HCDR2, and an HCDR3 as set forth in SEQ ID NO:170, and an LCDR1, an LCDR2, and an LCDR3 as set forth in SEQ ID NO:171;
p. an HCDR1, an HCDR2, and an HCDR3 as set forth in SEQ ID NO:172, and an LCDR1, an LCDR2, and an LCDR3 as set forth in SEQ ID NO:173;
q. an HCDR1, an HCDR2, and an HCDR3 as set forth in SEQ ID NO:174, and an LCDR1, an LCDR2, and an LCDR3 as set forth in SEQ ID NO:175;
r. an HCDR1, an HCDR2, and an HCDR3 as set forth in SEQ ID NO:176, and an LCDR1, an LCDR2, and an LCDR3 as set forth in SEQ ID NO:177;
s. an HCDR1, an HCDR2, and an HCDR3 as set forth in SEQ ID NO:178, and an LCDR1, an LCDR2, and an LCDR3 as set forth in SEQ ID NO:179;
t. an HCDR1, an HCDR2, and an HCDR3 as set forth in SEQ ID NO:180, and an LCDR1, an LCDR2, and an LCDR3 as set forth in SEQ ID NO:181;
u. an HCDR1, an HCDR2, and an HCDR3 as set forth in SEQ ID NO:182, and an LCDR1, an LCDR2, and an LCDR3 as set forth in SEQ ID NO:183;
v. an HCDR1, an HCDR2, and an HCDR3 as set forth in SEQ ID NO:186, and an LCDR1, an LCDR2, and an LCDR3 as set forth in SEQ ID NO:187;
w. an HCDR1, an HCDR2, and an HCDR3 as set forth in SEQ ID NO:188, and an LCDR1, an LCDR2, and an LCDR3 as set forth in SEQ ID NO:189;
x. an HCDR1, an HCDR2, and an HCDR3 as set forth in SEQ ID NO:190, and an LCDR1, an LCDR2, and an LCDR3 as set forth in SEQ ID NO:191;
y. an HCDR1, an HCDR2, and an HCDR3 as set forth in SEQ ID NO:192, and an LCDR1, an LCDR2, and an LCDR3 as set forth in SEQ ID NO:193;
z. an HCDR1, an HCDR2, and an HCDR3 as set forth in SEQ ID NO:194, and an LCDR1, an LCDR2, and an LCDR3 as set forth in SEQ ID NO:195;
aa. an HCDR1, an HCDR2, and an HCDR3 as set forth in SEQ ID NO:196, and an LCDR1, an LCDR2, and an LCDR3 as set forth in SEQ ID NO:197;
bb. an HCDR1, an HCDR2, and an HCDR3 as set forth in SEQ ID NO:198, and an LCDR1, an LCDR2, and an LCDR3 as set forth in SEQ ID NO:199;
cc. an HCDR1, an HCDR2, and an HCDR3 as set forth in SEQ ID NO:200, and an LCDR1, an LCDR2, and an LCDR3 as set forth in SEQ ID NO:201;
dd. an HCDR1, an HCDR2, and an HCDR3 as set forth in SEQ ID NO:202, and an LCDR1, an LCDR2, and an LCDR3 as set forth in SEQ ID NO:203;
ee. an HCDR1, an HCDR2, and an HCDR3 as set forth in SEQ ID NO:204, and an LCDR1, an LCDR2, and an LCDR3 as set forth in SEQ ID NO:205;
ff. an HCDR1, an HCDR2, and an HCDR3 as set forth in SEQ ID NO:206, and an LCDR1, an LCDR2, and an LCDR3 as set forth in SEQ ID NO:207;
gg. an HCDR1, an HCDR2, and an HCDR3 as set forth in SEQ ID NO:208, and an LCDR1, an LCDR2, and an LCDR3 as set forth in SEQ ID NO:209;
hh. an HCDR1, an HCDR2, and an HCDR3 as set forth in SEQ ID NO:210, and an LCDR1, an LCDR2, and an LCDR3 as set forth in SEQ ID NO:211;
ii. an HCDR1, an HCDR2, and an HCDR3 as set forth in SEQ ID NO:212, and an LCDR1, an LCDR2, and an LCDR3 as set forth in SEQ ID NO:213;
jj. an HCDR1, an HCDR2, and an HCDR3 as set forth in SEQ ID NO:214, and an LCDR1, an LCDR2, and an LCDR3 as set forth in SEQ ID NO:215;
kk. an HCDR1, an HCDR2, and an HCDR3 as set forth in SEQ ID NO:216, and an LCDR1, an LCDR2, and an LCDR3 as set forth in SEQ ID NO:217;
ll. an HCDR1, an HCDR2, and an HCDR3 as set forth in SEQ ID NO:218, and an LCDR1, an LCDR2, and an LCDR3 as set forth in SEQ ID NO:219;
mm. an HCDR1, an HCDR2, and an HCDR3 as set forth in SEQ ID NO:220, and an LCDR1, an LCDR2, and an LCDR3 as set forth in SEQ ID NO:221;
nn. an HCDR1, an HCDR2, and an HCDR3 as set forth in SEQ ID NO:222, and an LCDR1, an LCDR2, and an LCDR3 as set forth in SEQ ID NO:223;
oo. an HCDR1, an HCDR2, and an HCDR3 as set forth in SEQ ID NO:226, and an LCDR1, an LCDR2, and an LCDR3 as set forth in SEQ ID NO:227;
pp. an HCDR1, an HCDR2, and an HCDR3 as set forth in SEQ ID NO:228, and an LCDR1, an LCDR2, and an LCDR3 as set forth in SEQ ID NO:229;
qq. an HCDR1, an HCDR2, and an HCDR3 as set forth in SEQ ID NO:230, and an LCDR1, an LCDR2, and an LCDR3 as set forth in SEQ ID NO:231;
rr. an HCDR1, an HCDR2, and an HCDR3 as set forth in SEQ ID NO:232, and an LCDR1, an LCDR2, and an LCDR3 as set forth in SEQ ID NO:233;
ss. an HCDR1, an HCDR2, and an HCDR3 as set forth in SEQ ID NO:234, and an LCDR1, an LCDR2, and an LCDR3 as set forth in SEQ ID NO:235;
tt. an HCDR1, an HCDR2, and an HCDR3 as set forth in SEQ ID NO:236, and an LCDR1, an LCDR2, and an LCDR3 as set forth in SEQ ID NO:237;
uu. an HCDR1, an HCDR2, and an HCDR3 as set forth in SEQ ID NO:238, and an LCDR1, an LCDR2, and an LCDR3 as set forth in SEQ ID NO:239;
vv. an HCDR1, an HCDR2, and an HCDR3 as set forth in SEQ ID NO:240, and an LCDR1, an LCDR2, and an LCDR3 as set forth in SEQ ID NO:241;
ww. an HCDR1, an HCDR2, and an HCDR3 as set forth in SEQ ID NO:246, and an LCDR1, an LCDR2, and an LCDR3 as set forth in SEQ ID NO:247;
xx. an HCDR1, an HCDR2, and an HCDR3 as set forth in SEQ ID NO:248, and an LCDR1, an LCDR2, and an LCDR3 as set forth in SEQ ID NO:249;
yy. an HCDR1, an HCDR2, and an HCDR3 as set forth in SEQ ID NO:250, and an LCDR1, an LCDR2, and an LCDR3 as set forth in SEQ ID NO:251;
zz. an HCDR1, an HCDR2, and an HCDR3 as set forth in SEQ ID NO:252, and an LCDR1, an LCDR2, and an LCDR3 as set forth in SEQ ID NO:253;
aaa. an HCDR1, an HCDR2, and an HCDR3 as set forth in SEQ ID NO:254, and an LCDR1, an LCDR2, and an LCDR3 as set forth in SEQ ID NO:255;
bbb. an HCDR1, an HCDR2, and an HCDR3 as set forth in SEQ ID NO:256, and an LCDR1, an LCDR2, and an LCDR3 as set forth in SEQ ID NO:257;
ccc. an HCDR1, an HCDR2, and an HCDR3 as set forth in SEQ ID NO:258, and an LCDR1, an LCDR2, and an LCDR3 as set forth in SEQ ID NO:259;
ddd. an HCDR1, an HCDR2, and an HCDR3 as set forth in SEQ ID NO:260, and an LCDR1, an LCDR2, and an LCDR3 as set forth in SEQ ID NO:261;
eee. an HCDR1, an HCDR2, and an HCDR3 as set forth in SEQ ID NO:262, and a LCDR1, an LCDR2, and an LCDR3 as set forth in SEQ ID NO:263;
fff. an HCDR1, an HCDR2, and an HCDR3 as set forth in SEQ ID NO:264, and an LCDR1, an LCDR2, and an LCDR3 as set forth in SEQ ID NO:265;
ggg. an HCDR1, an HCDR2, and an HCDR3 as set forth in SEQ ID NO:268, and an LCDR1, an LCDR2, and an LCDR3 as set forth in SEQ ID NO:269;
hhh. an HCDR1, an HCDR2, and an HCDR3 as set forth in SEQ ID NO:786, and an LCDR1, an LCDR2, and an LCDR3 as set forth in SEQ ID NO:787;
iii. an HCDR1, an HCDR2, and an HCDR3 as set forth in SEQ ID NO:788, and an LCDR1, an LCDR2, and an LCDR3 as set forth in SEQ ID NO:789;
jjj. an HCDR1, an HCDR2, and an HCDR3 as set forth in SEQ ID NO:790, and an LCDR1, an LCDR2, and an LCDR3 as set forth in SEQ ID NO:791;
kkk. an HCDR1, an HCDR2, and an HCDR3 as set forth in SEQ ID NO:792, and an LCDR1, an LCDR2, and an LCDR3 as set forth in SEQ ID NO:793;
lll. an HCDR1, an HCDR2, and an HCDR3 as set forth in SEQ ID NO:798, and an LCDR1, an LCDR2, and an LCDR3 as set forth in SEQ ID NO:799;
mmm. an HCDR1, an HCDR2, and an HCDR3 as set forth in SEQ ID NO:806, and an LCDR1, an LCDR2, and an LCDR3 as set forth in SEQ ID NO:807;
nnn. an HCDR1, an HCDR2, and an HCDR3 as set forth in SEQ ID NO:808, and an LCDR1, an LCDR2, and an LCDR3 as set forth in SEQ ID NO:809;
ooo. an HCDR1, an HCDR2, and an HCDR3 as set forth in SEQ ID NO:810, and an LCDR1, an LCDR2, and an LCDR3 as set forth in SEQ ID NO:811;
ppp. an HCDR1, an HCDR2, and an HCDR3 as set forth in SEQ ID NO:812, and an LCDR1, an LCDR2, and an LCDR3 as set forth in SEQ ID NO:813;
qqq. an HCDR1, an HCDR2, and an HCDR3 as set forth in SEQ ID NO:814, and an LCDR1, an LCDR2, and an LCDR3 as set forth in SEQ ID NO:815;
rrr. an HCDR1, an HCDR2, and an HCDR3 as set forth in SEQ ID NO:816, and an LCDR1, an LCDR2, and an LCDR3 as set forth in SEQ ID NO:817;
sss. an HCDR1, an HCDR2, and an HCDR3 as set forth in SEQ ID NO:818, and an LCDR1, an LCDR2, and an LCDR3 as set forth in SEQ ID NO:819;
ttt. an HCDR1, an HCDR2, and an HCDR3 as set forth in SEQ ID NO:820, and an LCDR1, an LCDR2, and an LCDR3 as set forth in SEQ ID NO:821;
uuu. an HCDR1, an HCDR2, and an HCDR3 as set forth in SEQ ID NO:822, and an LCDR1, an LCDR2, and an LCDR3 as set forth in SEQ ID NO:823;
vvv. an HCDR1, an HCDR2, and an HCDR3 as set forth in SEQ ID NO:824, and an LCDR1, an LCDR2, and an LCDR3 as set forth in SEQ ID NO:825;
www. an HCDR1, an HCDR2, and an HCDR3 as set forth in SEQ ID NO:826, and an LCDR1, an LCDR2, and an LCDR3 as set forth in SEQ ID NO:827;
xxx. an HCDR1, an HCDR2, and an HCDR3 as set forth in SEQ ID NO:828, and an LCDR1, an LCDR2, and an LCDR3 as set forth in SEQ ID NO:829;
yyy. an HCDR1, an HCDR2, and an HCDR3 as set forth in SEQ ID NO:830, and an LCDR1, an LCDR2, and an LCDR3 as set forth in SEQ ID NO:831;
zzz. an HCDR1, an HCDR2, and an HCDR3 as set forth in SEQ ID NO:832, and an LCDR1, an LCDR2, and an LCDR3 as set forth in SEQ ID NO:833;
aaaa. an HCDR1, an HCDR2, and an HCDR3 as set forth in SEQ ID NO:836, and an LCDR1, an LCDR2, and an LCDR3 as set forth in SEQ ID NO:837;
bbbb. an HCDR1, an HCDR2, and an HCDR3 as set forth in SEQ ID NO:838, and an LCDR1, an LCDR2, and an LCDR3 as set forth in SEQ ID NO:839;
cccc. an HCDR1, an HCDR2, and an HCDR3 as set forth in SEQ ID NO:840, and an LCDR1, an LCDR2, and an LCDR3 as set forth in SEQ ID NO:841;
dddd. an HCDR1, an HCDR2, and an HCDR3 as set forth in SEQ ID NO: 842, and an LCDR1, an LCDR2, and an LCDR3 as set forth in SEQ ID NO:843;
eeee. an HCDR1, an HCDR2, and an HCDR3 as set forth in SEQ ID NO:846, and an LCDR1, an LCDR2, and an LCDR3 as set forth in SEQ ID NO:847;
ffff. an HCDR1, an HCDR2, and an HCDR3 as set forth in SEQ ID NO:848, and an LCDR1, an LCDR2, and an LCDR3 as set forth in SEQ ID NO:849;
gggg. an HCDR1, an HCDR2, and an HCDR3 as set forth in SEQ ID NO:850, and an LCDR1, an LCDR2, and an LCDR3 as set forth in SEQ ID NO:851;
hhhh. an HCDR1, an HCDR2, and an HCDR3 as set forth in SEQ ID NO:852, and an LCDR1, an LCDR2, and an LCDR3 as set forth in SEQ ID NO:853;
iiii. an HCDR1, an HCDR2, and an HCDR3 as set forth in SEQ ID NO:854, and an LCDR1, an LCDR2, and an LCDR3 as set forth in SEQ ID NO:855;
jjjj. an HCDR1, an HCDR2, and an HCDR3 as set forth in SEQ ID NO:856, and an LCDR1, an LCDR2, and an LCDR3 as set forth in SEQ ID NO:857;
kkkk. an HCDR1, an HCDR2, and an HCDR3 as set forth in SEQ ID NO:858, and an LCDR1, an LCDR2, and an LCDR3 as set forth in SEQ ID NO:859;
llll. an HCDR1, an HCDR2, and an HCDR3 as set forth in SEQ ID NO:860, and an LCDR1, an LCDR2, and an LCDR3 as set forth in SEQ ID NO:861;
mmmm. an HCDR1, an HCDR2, and an HCDR3 as set forth in SEQ ID NO:862, and an LCDR1, an LCDR2, and an LCDR3 as set forth in SEQ ID NO:863;
nnnn. an HCDR1, an HCDR2, and an HCDR3 as set forth in SEQ ID NO:864, and an LCDR1, an LCDR2, and an LCDR3 as set forth in SEQ ID NO:865;
oooo. an HCDR1, an HCDR2, and an HCDR3 as set forth in SEQ ID NO:866, and an LCDR1, an LCDR2, and an LCDR3 as set forth in SEQ ID NO:867;
pppp. an HCDR1, an HCDR2, and an HCDR3 as set forth in SEQ ID NO:868, and an LCDR1, an LCDR2, and an LCDR3 as set forth in SEQ ID NO:869;
qqqq. an HCDR1, an HCDR2, and an HCDR3 as set forth in SEQ ID NO:870, and an LCDR1, an LCDR2, and an LCDR3 as set forth in SEQ ID NO:871;
rrrr. an HCDR1, an HCDR2, and an HCDR3 as set forth in SEQ ID NO:872, and an LCDR1, an LCDR2, and an LCDR3 as set forth in SEQ ID NO:873;
ssss. an HCDR1, an HCDR2, and an HCDR3 as set forth in SEQ ID NO:874, and an LCDR1, an LCDR2, and an LCDR3 as set forth in SEQ ID NO:875;
tttt. an HCDR1, an HCDR2, and an HCDR3 as set forth in SEQ ID NO:876, and an LCDR1, an LCDR2, and an LCDR3 as set forth in SEQ ID NO:877;
uuuu. an HCDR1, an HCDR2, and an HCDR3 as set forth in SEQ ID NO:878, and an LCDR1, an LCDR2, and an LCDR3 as set forth in SEQ ID NO:879;
vvvv. an HCDR1, an HCDR2, and an HCDR3 as set forth in SEQ ID NO:880, and an LCDR1, an LCDR2, and an LCDR3 as set forth in SEQ ID NO:881;
wwww. an HCDR1, an HCDR2, and an HCDR3 as set forth in SEQ ID NO:882, and an LCDR1, an LCDR2, and an LCDR3 as set forth in SEQ ID NO:883;
xxxx. an HCDR1, an HCDR2, and an HCDR3 as set forth in SEQ ID NO:884, and an LCDR1, an LCDR2, and an LCDR3 as set forth in SEQ ID NO:885;
yyyy. an HCDR1, an HCDR2, and an HCDR3 as set forth in SEQ ID NO:886, and an LCDR1, an LCDR2, and an LCDR3 as set forth in SEQ ID NO:887;
zzzz. an HCDR1, an HCDR2, and an HCDR3 as set forth in SEQ ID NO:888, and an LCDR1, an LCDR2, and an LCDR3 as set forth in SEQ ID NO:889; or
aaaaa. an HCDR1, an HCDR2, and an HCDR3 as set forth in SEQ ID NO:890, and an LCDR1, an LCDR2, and an LCDR3 as set forth in SEQ ID NO:891.

In some embodiments, antibody or antigen binding fragment thereof is provided that binds IL-23R comprising:
a. a VH comprising an HCDR1, an HCDR2, and an HCDR3 having an amino acid sequence of SEQ ID NOs:276, 277, and 278, respectively, and a VL comprising an LCDR1, an LCDR2, and an LCDR3 having an amino acid sequence of SEQ ID NOs:279, 280, and 281, respectively;
b. a VH comprising an HCDR1, an HCDR2, and an HCDR3 having an amino acid sequence of SEQ ID NOs:288, 289, and 290, respectively, and a VL comprising an LCDR1, an LCDR2, and an LCDR3 having an amino acid sequence of SEQ ID NOs:291, 292, and 293, respectively;
c. a VH comprising an HCDR1, an HCDR2, and an HCDR3 having an amino acid sequence of SEQ ID NOs:294, 295, and 296, respectively, and a VL comprising an LCDR1, an LCDR2, and an LCDR3 having an amino acid sequence of SEQ ID NOs:297, 298, and 299, respectively;
d. a VH comprising an HCDR1, an HCDR2, and an HCDR3 having an amino acid sequence of SEQ ID NOs:306, 307, and 308, respectively, and a VL comprising an LCDR1, an LCDR2, and an LCDR3 having an amino acid sequence of SEQ ID NOs:309, 310, and 311, respectively;
e. a VH comprising an HCDR1, an HCDR2, and an HCDR3 having an amino acid sequence of SEQ ID NOs:312, 313, and 314, respectively, and a VL comprising an LCDR1, an LCDR2, and an LCDR3 having an amino acid sequence of SEQ ID NOs:315, 316, and 317, respectively;
f. a VH comprising an HCDR1, an HCDR2, and an HCDR3 having an amino acid sequence of SEQ ID NOs:318, 319, and 320, respectively, and a VL comprising an LCDR1, an LCDR2, and an LCDR3 having an amino acid sequence of SEQ ID NOs:321, 322, and 323, respectively;
g. a VH comprising an HCDR1, an HCDR2, and an HCDR3 having an amino acid sequence of SEQ ID NOs:324, 325, and 326, respectively, and a VL comprising an LCDR1, an LCDR2, and an LCDR3 having an amino acid sequence of SEQ ID NOs:327, 328, and 329, respectively;
h. a VH comprising an HCDR1, an HCDR2, and an HCDR3 having an amino acid sequence of SEQ ID NOs:330, 331, and 332, respectively, and a VL comprising an LCDR1, an LCDR2, and an LCDR3 having an amino acid sequence of SEQ ID NOs:333, 334, and 335, respectively;
i. a VH comprising an HCDR1, an HCDR2, and an HCDR3 having an amino acid sequence of SEQ ID NOs:336, 337, and 338, respectively, and a VL comprising an LCDR1, an LCDR2, and an LCDR3 having an amino acid sequence of SEQ ID NOs:339, 340, and 341, respectively;
j. a VH comprising an HCDR1, an HCDR2, and an HCDR3 having an amino acid sequence of SEQ ID NOs:342, 343, and 344, respectively, and a VL comprising an LCDR1, an LCDR2, and an LCDR3 having an amino acid sequence of SEQ ID NOs:345, 346, and 347, respectively;
k. a VH comprising an HCDR1, an HCDR2, and an HCDR3 having an amino acid sequence of SEQ ID NOs:348, 349, and 350, respectively, and a VL comprising an LCDR1, an LCDR2, and an LCDR3 having an amino acid sequence of SEQ ID NOs:351, 352, and 353, respectively;
l. a VH comprising an HCDR1, an HCDR2, and an HCDR3 having an amino acid sequence of SEQ ID NOs:354, 355, and 356, respectively, and a VL comprising an LCDR1, an LCDR2, and an LCDR3 having an amino acid sequence of SEQ ID NOs:357, 358, and 359, respectively;
m. a VH comprising an HCDR1, an HCDR2, and an HCDR3 having an amino acid sequence of SEQ ID NOs:360, 361, and 362, respectively, and a VL comprising an LCDR1, an LCDR2, and an LCDR3 having an amino acid sequence of SEQ ID NOs:363, 364, and 365, respectively;
n. a VH comprising an HCDR1, an HCDR2, and an HCDR3 having an amino acid sequence of SEQ ID NOs:366, 367, and 368, respectively, and a VL comprising an LCDR1, an LCDR2, and an LCDR3 having an amino acid sequence of SEQ ID NOs:369, 370, and 371, respectively;
o. a VH comprising an HCDR1, an HCDR2, and an HCDR3 having an amino acid sequence of SEQ ID NOs:372, 373, and 374, respectively, and a VL comprising an LCDR1, an LCDR2, and an LCDR3 having an amino acid sequence of SEQ ID NOs:375, 376, and 377, respectively;
p. a VH comprising an HCDR1, an HCDR2, and an HCDR3 having an amino acid sequence of SEQ ID NOs:378, 379, and 380, respectively, and a VL comprising an LCDR1, an LCDR2, and an LCDR3 having an amino acid sequence of SEQ ID NOs:381, 382, and 383, respectively;
q. a VH comprising an HCDR1, an HCDR2, and an HCDR3 having an amino acid sequence of SEQ ID NOs:384, 385, and 386, respectively, and a VL comprising an LCDR1, an LCDR2, and an LCDR3 having an amino acid sequence of SEQ ID NOs:387, 388, and 389, respectively;
r. a VH comprising an HCDR1, an HCDR2, and an HCDR3 having an amino acid sequence of SEQ ID NOs:390, 391, and 392, respectively, and a VL comprising an LCDR1, an LCDR2, and an LCDR3 having an amino acid sequence of SEQ ID NOs:393, 394, and 395, respectively;
s. a VH comprising an HCDR1, an HCDR2, and an HCDR3 having an amino acid sequence of SEQ ID NOs:396, 397, and 398, respectively, and a VL comprising an LCDR1, an LCDR2, and an LCDR3 having an amino acid sequence of SEQ ID NOs:399, 400, and 401, respectively;
t. a VH comprising an HCDR1, an HCDR2, and an HCDR3 having an amino acid sequence of SEQ ID NOs:402, 403, and 404, respectively, and a VL comprising an LCDR1, an LCDR2, and an LCDR3 having an amino acid sequence of SEQ ID NOs:405, 406, and 407, respectively;
u. a VH comprising an HCDR1, an HCDR2, and an HCDR3 having an amino acid sequence of SEQ ID NOs:408, 409, and 410, respectively, and a VL comprising an LCDR1, an LCDR2, and an LCDR3 having an amino acid sequence of SEQ ID NOs:411, 412, and 413, respectively;
v. a VH comprising an HCDR1, an HCDR2, and an HCDR3 having an amino acid sequence of SEQ ID NOs:420, 421, and 422, respectively, and a VL comprising an LCDR1, an LCDR2, and an LCDR3 having an amino acid sequence of SEQ ID NOs:423, 424, and 425, respectively;
w. a VH comprising an HCDR1, an HCDR2, and an HCDR3 having an amino acid sequence of SEQ ID NOs:426, 427, and 428, respectively, and a VL comprising an LCDR1, an LCDR2, and an LCDR3 having an amino acid sequence of SEQ ID NOs:429, 430, and 431, respectively;
x. a VH comprising an HCDR1, an HCDR2, and an HCDR3 having an amino acid sequence of SEQ ID NOs:432, 433, and 434, respectively, and a VL comprising an LCDR1, an LCDR2, and an LCDR3 having an amino acid sequence of SEQ ID NOs:435, 436, and 437, respectively;
y. a VH comprising an HCDR1, an HCDR2, and an HCDR3 having an amino acid sequence of SEQ ID NOs:438, 439, and 440, respectively, and a VL comprising an LCDR1, an LCDR2, and an LCDR3 having an amino acid sequence of SEQ ID NOs:441, 442, and 443, respectively;
z. a VH comprising an HCDR1, an HCDR2, and an HCDR3 having an amino acid sequence of SEQ ID NOs:444, 445, and 446, respectively, and a VL comprising an LCDR1, an LCDR2, and an LCDR3 having an amino acid sequence of SEQ ID NOs:447, 448, and 449, respectively;
aa. a VH comprising an HCDR1, an HCDR2, and an HCDR3 having an amino acid sequence of SEQ ID NOs:450, 451, and 452, respectively, and a VL comprising an LCDR1, an LCDR2, and an LCDR3 having an amino acid sequence of SEQ ID NOs:453, 454, and 455, respectively;
bb. a VH comprising an HCDR1, an HCDR2, and an HCDR3 having an amino acid sequence of SEQ ID NOs:456, 457, and 458, respectively, and a VL comprising an LCDR1, an LCDR2, and an LCDR3 having an amino acid sequence of SEQ ID NOs:459, 460, and 461, respectively;
cc. a VH comprising an HCDR1, an HCDR2, and an HCDR3 having an amino acid sequence of SEQ ID NOs:462, 463, and 464, respectively, and a VL comprising an LCDR1, an LCDR2, and an LCDR3 having an amino acid sequence of SEQ ID NOs:465, 466, and 467, respectively;
dd. a VH comprising an HCDR1, an HCDR2, and an HCDR3 having an amino acid sequence of SEQ ID NOs:468, 469, and 470, respectively, and a VL comprising an LCDR1, an LCDR2, and an LCDR3 having an amino acid sequence of SEQ ID NOs:471, 472, and 473, respectively;
ee. a VH comprising an HCDR1, an HCDR2, and an HCDR3 having an amino acid sequence of SEQ ID NOs:474, 475, and 476, respectively, and a VL comprising an LCDR1, an LCDR2, and an LCDR3 having an amino acid sequence of SEQ ID NOs:477, 478, and 479, respectively;
ff. a VH comprising an HCDR1, an HCDR2, and an HCDR3 having an amino acid sequence of SEQ ID NOs:480, 481, and 482, respectively, and a VL comprising an LCDR1, an LCDR2, and an LCDR3 having an amino acid sequence of SEQ ID NOs:483, 484, and 485, respectively;
gg. a VH comprising an HCDR1, an HCDR2, and an HCDR3 having an amino acid sequence of SEQ ID NOs:486, 487, and 488, respectively, and a VL comprising an LCDR1, an LCDR2, and an LCDR3 having an amino acid sequence of SEQ ID NOs:489, 490, and 491, respectively;
hh. a VH comprising an HCDR1, an HCDR2, and an HCDR3 having an amino acid sequence of SEQ ID NOs:492, 493, and 494, respectively, and a VL comprising an LCDR1, an LCDR2, and an LCDR3 having an amino acid sequence of SEQ ID NOs:495, 496, and 497, respectively;
ii. a VH comprising an HCDR1, an HCDR2, and an HCDR3 having an amino acid sequence of SEQ ID NOs:498, 499, and 500, respectively, and a VL comprising an LCDR1, an LCDR2, and an LCDR3 having an amino acid sequence of SEQ ID NOs:501, 502, and 503, respectively;
jj. a VH comprising an HCDR1, an HCDR2, and an HCDR3 having an amino acid sequence of SEQ ID NOs:504, 505, and 506, respectively, and a VL comprising an LCDR1, an LCDR2, and an LCDR3 having an amino acid sequence of SEQ ID NOs:507, 508, and 509, respectively;
kk. a VH comprising an HCDR1, an HCDR2, and an HCDR3 having an amino acid sequence of SEQ ID NOs:510, 511, and 512, respectively, and a VL comprising an LCDR1, an LCDR2, and an LCDR3 having an amino acid sequence of SEQ ID NOs:513, 514, and 515, respectively;
ll. a VH comprising an HCDR1, an HCDR2, and an HCDR3 having an amino acid sequence of SEQ ID NOs:516, 517, and 518, respectively, and a VL comprising an LCDR1, an LCDR2, and an LCDR3 having an amino acid sequence of SEQ ID NOs:519, 520, and 521, respectively;
mm. a VH comprising an HCDR1, an HCDR2, and an HCDR3 having an amino acid sequence of SEQ ID NOs:522, 523, and 524, respectively, and a VL comprising an LCDR1, an LCDR2, and an LCDR3 having an amino acid sequence of SEQ ID NOs:525, 526, and 527, respectively;
nn. a VH comprising an HCDR1, an HCDR2, and an HCDR3 having an amino acid sequence of SEQ ID NOs:528, 529, and 530, respectively, and a VL comprising an LCDR1, an LCDR2, and an LCDR3 having an amino acid sequence of SEQ ID NOs:531, 532, and 533, respectively;
oo. a VH comprising an HCDR1, an HCDR2, and an HCDR3 having an amino acid sequence of SEQ ID NOs:540, 541, and 542, respectively, and a VL comprising an LCDR1, an LCDR2, and an LCDR3 having an amino acid sequence of SEQ ID NOs:543, 544, and 545, respectively;
pp. a VH comprising an HCDR1, an HCDR2, and an HCDR3 having an amino acid sequence of SEQ ID NOs:546, 547, and 548, respectively, and a VL comprising an LCDR1, an LCDR2, and an LCDR3 having an amino acid sequence of SEQ ID NOs:549, 550, and 551, respectively;
qq. a VH comprising an HCDR1, an HCDR2, and an HCDR3 having an amino acid sequence of SEQ ID NOs:552, 553, and 554, respectively, and a VL comprising an LCDR1, an LCDR2, and an LCDR3 having an amino acid sequence of SEQ ID NOs:555, 556, and 557, respectively;
rr. a VH comprising an HCDR1, an HCDR2, and an HCDR3 having an amino acid sequence of SEQ ID NOs:558, 559, and 560, respectively, and a VL comprising an LCDR1, an LCDR2, and an LCDR3 having an amino acid sequence of SEQ ID NOs:561, 562, and 563, respectively;
ss. a VH comprising an HCDR1, an HCDR2, and an HCDR3 having an amino acid sequence of SEQ ID NOs:564, 565, and 566, respectively, and a VL comprising an LCDR1, an LCDR2, and an LCDR3 having an amino acid sequence of SEQ ID NOs:567, 568, and 569, respectively;
tt. a VH comprising an HCDR1, an HCDR2, and an HCDR3 having an amino acid sequence of SEQ ID NOs:570, 571, and 572, respectively, and a VL comprising an LCDR1, an LCDR2, and an LCDR3 having an amino acid sequence of SEQ ID NOs:573, 574, and 575, respectively;
uu. a VH comprising an HCDR1, an HCDR2, and an HCDR3 having an amino acid sequence of SEQ ID NOs:576, 577, and 578, respectively, and a VL comprising an LCDR1, an LCDR2, and an LCDR3 having an amino acid sequence of SEQ ID NOs:579, 580, and 581, respectively;
vv. a VH comprising an HCDR1, an HCDR2, and an HCDR3 having an amino acid sequence of SEQ ID NOs:582, 583, and 584, respectively, and a VL comprising an LCDR1, an LCDR2, and an LCDR3 having an amino acid sequence of SEQ ID NOs:585, 586, and 587, respectively;
ww. a VH comprising an HCDR1, an HCDR2, and an HCDR3 having an amino acid sequence of SEQ ID NOs:600, 601, and 602, respectively, and a VL comprising an LCDR1, an LCDR2, and an LCDR3 having an amino acid sequence of SEQ ID NOs:603, 604, and 605, respectively;
xx. a VH comprising an HCDR1, an HCDR2, and an HCDR3 having an amino acid sequence of SEQ ID NOs:606, 607, and 608, respectively, and a VL comprising an LCDR1, an LCDR2, and an LCDR3 having an amino acid sequence of SEQ ID NOs:609, 610, and 611, respectively;
yy. a VH comprising an HCDR1, an HCDR2, and an HCDR3 having an amino acid sequence of SEQ ID NOs:612, 613, and 614, respectively, and a VL comprising an LCDR1, an LCDR2, and an LCDR3 having an amino acid sequence of SEQ ID NOs:615, 616, and 617, respectively;
zz. a VH comprising an HCDR1, an HCDR2, and an HCDR3 having an amino acid sequence of SEQ ID NOs:618, 619, and 620, respectively, and a VL comprising an LCDR1, an LCDR2, and an LCDR3 having an amino acid sequence of SEQ ID NOs:621, 622, and 623, respectively;
aaa. a VH comprising an HCDR1, an HCDR2, and an HCDR3 having an amino acid sequence of SEQ ID NOs:624, 625, and 626, respectively, and a VL comprising an LCDR1, an LCDR2, and an LCDR3 having an amino acid sequence of SEQ ID NOs:627, 628, and 629, respectively;
bbb. a VH comprising an HCDR1, an HCDR2, and an HCDR3 having an amino acid sequence of SEQ ID NOs:630, 631, and 632, respectively, and a VL comprising an LCDR1, an LCDR2, and an LCDR3 having an amino acid sequence of SEQ ID NOs:633, 634, and 635, respectively;
ccc. a VH comprising an HCDR1, an HCDR2, and an HCDR3 having an amino acid sequence of SEQ ID NOs:636, 637, and 638, respectively, and a VL comprising an LCDR1, an LCDR2, and an LCDR3 having an amino acid sequence of SEQ ID NOs:639, 640, and 641, respectively;
ddd. a VH comprising an HCDR1, an HCDR2, and an HCDR3 having an amino acid sequence of SEQ ID NOs:642, 643, and 644, respectively, and a VL comprising an LCDR1, an LCDR2, and an LCDR3 having an amino acid sequence of SEQ ID NOs:645, 646, and 647, respectively;
eee. a VH comprising an HCDR1, an HCDR2, and an HCDR3 having an amino acid sequence of SEQ ID NOs:648, 649, and 650, respectively, and a VL comprising an LCDR1, an LCDR2, and an LCDR3 having an amino acid sequence of SEQ ID NOs:651, 652, and 653, respectively;
fff. a VH comprising an HCDR1, an HCDR2, and an HCDR3 having an amino acid sequence of SEQ ID NOs:654, 655, and 656, respectively, and a VL comprising an LCDR1, an LCDR2, and an LCDR3 having an amino acid sequence of SEQ ID NOs:657, 658, and 659, respectively;
ggg. a VH comprising an HCDR1, an HCDR2, and an HCDR3 having an amino acid sequence of SEQ ID NOs:666, 667, and 668, respectively, and a VL comprising an LCDR1, an LCDR2, and an LCDR3 having an amino acid sequence of SEQ ID NOs:669, 670, and 671, respectively;
hhh. a VH comprising an HCDR1, an HCDR2, and an HCDR3 having an amino acid sequence of SEQ ID NOs:900, 901, and 902, respectively, and a VL comprising an LCDR1, an LCDR2, and an LCDR3 having an amino acid sequence of SEQ ID NOs:903, 904, and 905, respectively;
iii. a VH comprising an HCDR1, an HCDR2, and an HCDR3 having an amino acid sequence of SEQ ID NOs:906, 907, and 908, respectively, and a VL comprising an LCDR1, an LCDR2, and an LCDR3 having an amino acid sequence of SEQ ID NOs:909, 910, and 911, respectively;
jjj. a VH comprising an HCDR1, an HCDR2, and an HCDR3 having an amino acid sequence of SEQ ID NOs:912, 913, and 914, respectively, and a VL comprising an LCDR1, an LCDR2, and an LCDR3 having an amino acid sequence of SEQ ID NOs:915, 916, and 917, respectively;
kkk. a VH comprising an HCDR1, an HCDR2, and an HCDR3 having an amino acid sequence of SEQ ID NOs:918, 919, and 920, respectively, and a VL comprising an LCDR1, an LCDR2, and an LCDR3 having an amino acid sequence of SEQ ID NOs:921, 922, and 923, respectively;
lll. a VH comprising an HCDR1, an HCDR2, and an HCDR3 having an amino acid sequence of SEQ ID NOs:936, 937, and 938, respectively, and a VL comprising an LCDR1, an LCDR2, and an LCDR3 having an amino acid sequence of SEQ ID NOs:939, 940, and 941, respectively;
mmm. a VH comprising an HCDR1, an HCDR2, and an HCDR3 having an amino acid sequence of SEQ ID NOs:960, 961, and 962, respectively, and a VL comprising an LCDR1, an LCDR2, and an LCDR3 having an amino acid sequence of SEQ ID NOs:963, 964, and 965, respectively;
nnn. a VH comprising an HCDR1, an HCDR2, and an HCDR3 having an amino acid sequence of SEQ ID NOs:966, 967, and 968, respectively, and a VL comprising an LCDR1, an LCDR2, and an LCDR3 having an amino acid sequence of SEQ ID NOs:969, 970, and 971, respectively;
ooo. a VH comprising an HCDR1, an HCDR2, and an HCDR3 having an amino acid sequence of SEQ ID NOs:972, 973, and 974, respectively, and a VL comprising an LCDR1, an LCDR2, and an LCDR3 having an amino acid sequence of SEQ ID NOs:975, 976, and 977, respectively;
ppp. a VH comprising an HCDR1, an HCDR2, and an HCDR3 having an amino acid sequence of SEQ ID NOs:978, 979, and 980, respectively, and a VL comprising an LCDR1, an LCDR2, and an LCDR3 having an amino acid sequence of SEQ ID NOs:981, 982, and 983, respectively;
qqq. a VH comprising an HCDR1, an HCDR2, and an HCDR3 having an amino acid sequence of SEQ ID NOs:984, 985, and 986, respectively, and a VL comprising an LCDR1, an LCDR2, and an LCDR3 having an amino acid sequence of SEQ ID NOs:987, 988, and 989, respectively;
rrr. a VH comprising an HCDR1, an HCDR2, and an HCDR3 having an amino acid sequence of SEQ ID NOs:990, 991, and 992, respectively, and a VL comprising an LCDR1, an LCDR2, and an LCDR3 having an amino acid sequence of SEQ ID NOs:993, 994, and 995, respectively;
sss. a VH comprising an HCDR1, an HCDR2, and an HCDR3 having an amino acid sequence of SEQ ID NOs:996, 997, and 998, respectively, and a VL comprising an LCDR1, an LCDR2, and an LCDR3 having an amino acid sequence of SEQ ID NOs:999, 1000, and 1001, respectively;
ttt. a VH comprising an HCDR1, an HCDR2, and an HCDR3 having an amino acid sequence of SEQ ID NOs:1002, 1003, and 1004, respectively, and a VL comprising an LCDR1, an LCDR2, and an LCDR3 having an amino acid sequence of SEQ ID NOs:1005, 1006, and 1007, respectively;
uuu. a VH comprising an HCDR1, an HCDR2, and an HCDR3 having an amino acid sequence of SEQ ID NOs:1008, 1009, and 1010, respectively, and a VL comprising an LCDR1, an LCDR2, and an LCDR3 having an amino acid sequence of SEQ ID NOs:1011, 1012, and 1013, respectively;
vvv. a VH comprising an HCDR1, an HCDR2, and an HCDR3 having an amino acid sequence of SEQ ID NOs:1014, 1015, and 1016, respectively, and a VL comprising an LCDR1, an LCDR2, and an LCDR3 having an amino acid sequence of SEQ ID NOs:1017, 1018, and 1019, respectively;
www.a VH comprising an HCDR1, an HCDR2, and an HCDR3 having an amino acid sequence of SEQ ID NOs:1020, 1021, and 1022, respectively, and a VL comprising an LCDR1, an LCDR2, and an LCDR3 having an amino acid sequence of SEQ ID NOs:1023, 1024, and 1025, respectively;
xxx. a VH comprising an HCDR1, an HCDR2, and an HCDR3 having an amino acid sequence of SEQ ID NOs:1026, 1027, and 1028, respectively, and a VL comprising an LCDR1, an LCDR2, and an LCDR3 having an amino acid sequence of SEQ ID NOs:1029, 1030, and 1031, respectively;
yyy. a VH comprising an HCDR1, an HCDR2, and an HCDR3 having an amino acid sequence of SEQ ID NOs:1032, 1033, and 1034, respectively, and a VL comprising an LCDR1, an LCDR2, and an LCDR3 having an amino acid sequence of SEQ ID NOs:1035, 1036, and 1037, respectively;
zzz. a VH comprising an HCDR1, an HCDR2, and an HCDR3 having an amino acid sequence of SEQ ID NOs:1038, 1039, and 1040, respectively, and a VL comprising an LCDR1, an LCDR2, and an LCDR3 having an amino acid sequence of SEQ ID NOs:1041, 1042, and 1043, respectively;
aaaa. a VH comprising an HCDR1, an HCDR2, and an HCDR3 having an amino acid sequence of SEQ ID NOs:1050, 1051, and 1052, respectively, and a VL comprising an LCDR1, an LCDR2, and an LCDR3 having an amino acid sequence of SEQ ID NOs:1053, 1054, and 1055, respectively;
bbbb. a VH comprising an HCDR1, an HCDR2, and an HCDR3 having an amino acid sequence of SEQ ID NOs:1056, 1057, and 1058, respectively, and a VL comprising an LCDR1, an LCDR2, and an LCDR3 having an amino acid sequence of SEQ ID NOs:1059, 1060, and 1061, respectively;
cccc. a VH comprising an HCDR1, an HCDR2, and an HCDR3 having an amino acid sequence of SEQ ID NOs:1062, 1063, and 1064, respectively, and a VL comprising an LCDR1, an LCDR2, and an LCDR3 having an amino acid sequence of SEQ ID NOs:1065, 1066, and 1067, respectively;
dddd. a VH comprising an HCDR1, an HCDR2, and an HCDR3 having an amino acid sequence of SEQ ID NOs:1068, 1069, and 1070, respectively, and a VL comprising an LCDR1, an LCDR2, and an LCDR3 having an amino acid sequence of SEQ ID NOs:1071, 1072, and 1073, respectively;
eeee. a VH comprising an HCDR1, an HCDR2, and an HCDR3 having an amino acid sequence of SEQ ID NOs:1080, 1081, and 1082, respectively, and a VL comprising an LCDR1, an LCDR2, and an LCDR3 having an amino acid sequence of SEQ ID NOs:1083, 1084, and 1085, respectively;
ffff. a VH comprising an HCDR1, an HCDR2, and an HCDR3 having an amino acid sequence of SEQ ID NOs:1086, 1087, and 1088, respectively, and a VL comprising an LCDR1, an LCDR2, and an LCDR3 having an amino acid sequence of SEQ ID NOs:1089, 1090, and 1091, respectively;
gggg. a VH comprising an HCDR1, an HCDR2, and an HCDR3 having an amino acid sequence of SEQ ID NOs:1092, 1093, and 1094, respectively, and a VL comprising an LCDR1, an LCDR2, and an LCDR3 having an amino acid sequence of SEQ ID NOs:1095, 1096, and 1097, respectively;
hhhh. a VH comprising an HCDR1, an HCDR2, and an HCDR3 having an amino acid sequence of SEQ ID NOs:1098, 1099, and 1100, respectively, and a VL comprising an LCDR1, an LCDR2, and an LCDR3 having an amino acid sequence of SEQ ID NOs:1101, 1102, and 1103, respectively;
iiii. a VH comprising an HCDR1, an HCDR2, and an HCDR3 having an amino acid sequence of SEQ ID NOs:1104, 1105, and 1106, respectively, and a VL comprising an LCDR1, an LCDR2, and an LCDR3 having an amino acid sequence of SEQ ID NOs:1107, 1108, and 1109, respectively;
jjjj. a VH comprising an HCDR1, an HCDR2, and an HCDR3 having an amino acid sequence of SEQ ID NOs:1110, 1111, and 1112, respectively, and a VL comprising an LCDR1, an LCDR2, and an LCDR3 having an amino acid sequence of SEQ ID NOs:1113, 1114, and 1115, respectively;
kkkk. a VH comprising an HCDR1, an HCDR2, and an HCDR3 having an amino acid sequence of SEQ ID NOs:1116, 1117, and 1118, respectively, and a VL comprising an LCDR1, an LCDR2, and an LCDR3 having an amino acid sequence of SEQ ID NOs:1119, 1120, and 1121, respectively;
llll. a VH comprising an HCDR1, an HCDR2, and an HCDR3 having an amino acid sequence of SEQ ID NOs:1122, 1123, and 1124, respectively, and a VL comprising an LCDR1, an LCDR2, and an LCDR3 having an amino acid sequence of SEQ ID NOs:1125, 1126, and 1127, respectively;
mmmm. a VH comprising an HCDR1, an HCDR2, and an HCDR3 having an amino acid sequence of SEQ ID NOs:1128, 1129, and 1130, respectively, and a VL comprising an LCDR1, an LCDR2, and an LCDR3 having an amino acid sequence of SEQ ID NOs:1131, 1132, and 1133, respectively;
nnnn. a VH comprising an HCDR1, an HCDR2, and an HCDR3 having an amino acid sequence of SEQ ID NOs:1134, 1135, and 1136, respectively, and a VL comprising an LCDR1, an LCDR2, and an LCDR3 having an amino acid sequence of SEQ ID NOs:1137, 1138, and 1139, respectively;
oooo. a VH comprising an HCDR1, an HCDR2, and an HCDR3 having an amino acid sequence of SEQ ID NOs:1140, 1141, and 1142, respectively, and a VL comprising an LCDR1, an LCDR2, and an LCDR3 having an amino acid sequence of SEQ ID NOs:1143, 1144, and 1145, respectively;
pppp. a VH comprising an HCDR1, an HCDR2, and an HCDR3 having an amino acid sequence of SEQ ID NOs:1146, 1147, and 1148, respectively, and a VL comprising an LCDR1, an LCDR2, and an LCDR3 having an amino acid sequence of SEQ ID NOs:1149, 1150, and 1151, respectively;
qqqq. a VH comprising an HCDR1, an HCDR2, and an HCDR3 having an amino acid sequence of SEQ ID NOs:1152, 1153, and 1154, respectively, and a VL comprising an LCDR1, an LCDR2, and an LCDR3 having an amino acid sequence of SEQ ID NOs:1155, 1156, and 1157, respectively;
rrrr. a VH comprising an HCDR1, an HCDR2, and an HCDR3 having an amino acid sequence of SEQ ID NOs:1158, 1159, and 1160, respectively, and a VL comprising an LCDR1, an LCDR2, and an LCDR3 having an amino acid sequence of SEQ ID NOs:1161, 1162, and 1163, respectively;
ssss. a VH comprising an HCDR1, an HCDR2, and an HCDR3 having an amino acid sequence of SEQ ID NOs:1164, 1165, and 1166, respectively, and a VL comprising an LCDR1, an LCDR2, and an LCDR3 having an amino acid sequence of SEQ ID NOs:1167, 1168, and 1169, respectively;
tttt. a VH comprising an HCDR1, an HCDR2, and an HCDR3 having an amino acid sequence of SEQ ID NOs:1170, 1171, and 1172, respectively, and a VL comprising an LCDR1, an LCDR2, and an LCDR3 having an amino acid sequence of SEQ ID NOs:1173, 1174, and 1175, respectively;
uuuu. a VH comprising an HCDR1, an HCDR2, and an HCDR3 having an amino acid sequence of SEQ ID NOs:1176, 1177, and 1178, respectively, and a VL comprising an LCDR1, an LCDR2, and an LCDR3 having an amino acid sequence of SEQ ID NOs:1179, 1180, and 1181, respectively;
vvvv. a VH comprising an HCDR1, an HCDR2, and an HCDR3 having an amino acid sequence of SEQ ID NOs:1182, 1183, and 1184, respectively, and a VL comprising an LCDR1, an LCDR2, and an LCDR3 having an amino acid sequence of SEQ ID NOs:1185, 1186, and 1187, respectively;
wwww. a VH comprising an HCDR1, an HCDR2, and an HCDR3 having an amino acid sequence of SEQ ID NOs:1188, 1189, and 1190, respectively, and a VL comprising an LCDR1, an LCDR2, and an LCDR3 having an amino acid sequence of SEQ ID NOs:1191, 1192, and 1193, respectively;
xxxx. a VH comprising an HCDR1, an HCDR2, and an HCDR3 having an amino acid sequence of SEQ ID NOs:1194, 1195, and 1196, respectively, and a VL comprising an LCDR1, an LCDR2, and an LCDR3 having an amino acid sequence of SEQ ID NOs:1197, 1198, and 1199, respectively;
yyyy. a VH comprising an HCDR1, an HCDR2, and an HCDR3 having an amino acid sequence of SEQ ID NOs:1200, 1201, and 1202, respectively, and a VL comprising an LCDR1, an LCDR2, and an LCDR3 having an amino acid sequence of SEQ ID NOs:1203, 1204, and 1205, respectively;
zzzz. a VH comprising an HCDR1, an HCDR2, and an HCDR3 having an amino acid sequence of SEQ ID NOs:1206, 1207, and 1208, respectively, and a VL comprising an LCDR1, an LCDR2, and an LCDR3 having an amino acid sequence of SEQ ID NOs:1209, 1210, and 1211, respectively; or
aaaaa. a VH comprising an HCDR1, an HCDR2, and an HCDR3 having an amino acid sequence of SEQ ID NOs:1212, 1213, and 1214, respectively, and a VL comprising an LCDR1, an LCDR2, and an LCDR3 having an amino acid sequence of SEQ ID NOs:1215, 1216, and 1217, respectively.

In some embodiments, antibody or antigen binding fragment thereof of is provided comprising:
a. a VH comprising the amino acid sequence of SEQ ID NO:138, and a VL comprising the amino acid sequence of SEQ ID NO:139;
b. a VH comprising the amino acid sequence of SEQ ID NO:142, and a VL comprising the amino acid sequence of SEQ ID NO:143;
c. a VH comprising the amino acid sequence of SEQ ID NO:144, and a VL comprising the amino acid sequence of SEQ ID NO:145;
d. a VH comprising the amino acid sequence of SEQ ID NO:148, and a VL comprising the amino acid sequence of SEQ ID NO:149;
e. a VH comprising the amino acid sequence of SEQ ID NO:150, and a VL comprising the amino acid sequence of SEQ ID NO:151;
f. a VH comprising the amino acid sequence of SEQ ID NO:152, and a VL comprising the amino acid sequence of SEQ ID NO:153;
g. a VH comprising the amino acid sequence of SEQ ID NO:154, and a VL comprising the amino acid sequence of SEQ ID NO:155;
h. a VH comprising the amino acid sequence of SEQ ID NO:156, and a VL comprising the amino acid sequence of SEQ ID NO:157;
i. a VH comprising the amino acid sequence of SEQ ID NO:158, and a VL comprising the amino acid sequence of SEQ ID NO:159;
j. a VH comprising the amino acid sequence of SEQ ID NO:160, and a VL comprising the amino acid sequence of SEQ ID NO:161;
k. a VH comprising the amino acid sequence of SEQ ID NO:162, and a VL comprising the amino acid sequence of SEQ ID NO:163;
l. a VH comprising the amino acid sequence of SEQ ID NO:164, and a VL comprising the amino acid sequence of SEQ ID NO:165;
m. a VH comprising the amino acid sequence of SEQ ID NO:166, and a VL comprising the amino acid sequence of SEQ ID NO:167;
n. a VH comprising the amino acid sequence of SEQ ID NO:168, and a VL comprising the amino acid sequence of SEQ ID NO:169;
o. a VH comprising the amino acid sequence of SEQ ID NO:170, and a VL comprising the amino acid sequence of SEQ ID NO:171;
p. a VH comprising the amino acid sequence of SEQ ID NO:172, and a VL comprising the amino acid sequence of SEQ ID NO:173;
q. a VH comprising the amino acid sequence of SEQ ID NO:174, and a VL comprising the amino acid sequence of SEQ ID NO:175;
r. a VH comprising the amino acid sequence of SEQ ID NO:176, and a VL comprising the amino acid sequence of SEQ ID NO:177;
s. a VH comprising the amino acid sequence of SEQ ID NO:178, and a VL comprising the amino acid sequence of SEQ ID NO:179;
t. a VH comprising the amino acid sequence of SEQ ID NO:180, and a VL comprising the amino acid sequence of SEQ ID NO:181;
u. a VH comprising the amino acid sequence of SEQ ID NO:182, and a VL comprising the amino acid sequence of SEQ ID NO:183;
v. a VH comprising the amino acid sequence of SEQ ID NO:186, and a VL comprising the amino acid sequence of SEQ ID NO:187;
w. a VH comprising the amino acid sequence of SEQ ID NO:188, and a VL comprising the amino acid sequence of SEQ ID NO:189;
x. a VH comprising the amino acid sequence of SEQ ID NO:190, and a VL comprising the amino acid sequence of SEQ ID NO:191;
y. a VH comprising the amino acid sequence of SEQ ID NO:192, and a VL comprising the amino acid sequence of SEQ ID NO:193;
z. a VH comprising the amino acid sequence of SEQ ID NO:194, and a VL comprising the amino acid sequence of SEQ ID NO:195;
aa. a VH comprising the amino acid sequence of SEQ ID NO:196, and a VL comprising the amino acid sequence of SEQ ID NO:197;
bb. a VH comprising the amino acid sequence of SEQ ID NO:198, and a VL comprising the amino acid sequence of SEQ ID NO:199;
cc. a VH comprising the amino acid sequence of SEQ ID NO:200, and a VL comprising the amino acid sequence of SEQ ID NO:201;
dd. a VH comprising the amino acid sequence of SEQ ID NO:202, and a VL comprising the amino acid sequence of SEQ ID NO:203;
ee. a VH comprising the amino acid sequence of SEQ ID NO:204, and a VL comprising the amino acid sequence of SEQ ID NO:205;
ff. a VH comprising the amino acid sequence of SEQ ID NO:206, and a VL comprising the amino acid sequence of SEQ ID NO:207;
gg. a VH comprising the amino acid sequence of SEQ ID NO:208, and a VL comprising the amino acid sequence of SEQ ID NO:209;
hh. a VH comprising the amino acid sequence of SEQ ID NO:210, and a VL comprising the amino acid sequence of SEQ ID NO:211;
ii. a VH comprising the amino acid sequence of SEQ ID NO:212, and a VL comprising the amino acid sequence of SEQ ID NO:213;
jj. a VH comprising the amino acid sequence of SEQ ID NO:214, and a VL comprising the amino acid sequence of SEQ ID NO:215;
kk. a VH comprising the amino acid sequence of SEQ ID NO:216, and a VL comprising the amino acid sequence of SEQ ID NO:217;
ll. a VH comprising the amino acid sequence of SEQ ID NO:218, and a VL comprising the amino acid sequence of SEQ ID NO:219;
mm. a VH comprising the amino acid sequence of SEQ ID NO:220, and a VL comprising the amino acid sequence of SEQ ID NO:221;
nn. a VH comprising the amino acid sequence of SEQ ID NO:222, and a VL comprising the amino acid sequence of SEQ ID NO:223;
oo. a VH comprising the amino acid sequence of SEQ ID NO:226, and a VL comprising the amino acid sequence of SEQ ID NO:227;
pp. a VH comprising the amino acid sequence of SEQ ID NO:228, and a VL comprising the amino acid sequence of SEQ ID NO:229;
qq. a VH comprising the amino acid sequence of SEQ ID NO:230, and a VL comprising the amino acid sequence of SEQ ID NO:231;
rr. a VH comprising the amino acid sequence of SEQ ID NO:232, and a VL comprising the amino acid sequence of SEQ ID NO:233;
ss. a VH comprising the amino acid sequence of SEQ ID NO:234, and a VL comprising the amino acid sequence of SEQ ID NO:235;
tt. a VH comprising the amino acid sequence of SEQ ID NO:236, and a VL comprising the amino acid sequence of SEQ ID NO:237;
uu. a VH comprising the amino acid sequence of SEQ ID NO:238, and a VL comprising the amino acid sequence of SEQ ID NO:239;
vv. a VH comprising the amino acid sequence of SEQ ID NO:240, and a VL comprising the amino acid sequence of SEQ ID NO:241;
ww. a VH comprising the amino acid sequence of SEQ ID NO:246, and a VL comprising the amino acid sequence of SEQ ID NO:247;
xx. a VH comprising the amino acid sequence of SEQ ID NO:248, and a VL comprising the amino acid sequence of SEQ ID NO:249;
yy. a VH comprising the amino acid sequence of SEQ ID NO:250, and a VL comprising the amino acid sequence of SEQ ID NO:251;
zz. a VH comprising the amino acid sequence of SEQ ID NO:252, and a VL comprising the amino acid sequence of SEQ ID NO:253;
aaa. a VH comprising the amino acid sequence of SEQ ID NO:254, and a VL comprising the amino acid sequence of SEQ ID NO:255;
bbb. a VH comprising the amino acid sequence of SEQ ID NO:256, and a VL comprising the amino acid sequence of SEQ ID NO:257;
ccc. a VH comprising the amino acid sequence of SEQ ID NO:258, and a VL comprising the amino acid sequence of SEQ ID NO:259;
ddd. a VH comprising the amino acid sequence of SEQ ID NO:260, and a VL comprising the amino acid sequence of SEQ ID NO:261;
eee. a VH comprising the amino acid sequence of SEQ ID NO:262, and a VL comprising the amino acid sequence of SEQ ID NO:263;
fff. a VH comprising the amino acid sequence of SEQ ID NO:264, and a VL comprising the amino acid sequence of SEQ ID NO:265;
ggg. a VH comprising the amino acid sequence of SEQ ID NO:268, and a VL comprising the amino acid sequence of SEQ ID NO:269;
hhh. a VH comprising the amino acid sequence of SEQ ID NO:786, and a VL comprising the amino acid sequence of SEQ ID NO:787;
iii. a VH comprising the amino acid sequence of SEQ ID NO:788, and a VL comprising the amino acid sequence of SEQ ID NO:789;
jjj. a VH comprising the amino acid sequence of SEQ ID NO:790, and a VL comprising the amino acid sequence of SEQ ID NO:791;
kkk. a VH comprising the amino acid sequence of SEQ ID NO:792, and a VL comprising the amino acid sequence of SEQ ID NO:793;
lll. a VH comprising the amino acid sequence of SEQ ID NO:798, and a VL comprising the amino acid sequence of SEQ ID NO:799;
mmm. a VH comprising the amino acid sequence of SEQ ID NO:806, and a VL comprising the amino acid sequence of SEQ ID NO:807;
nnn. a VH comprising the amino acid sequence of SEQ ID NO:808, and a VL comprising the amino acid sequence of SEQ ID NO:809;
ooo. a VH comprising the amino acid sequence of SEQ ID NO:810, and a VL comprising the amino acid sequence of SEQ ID NO:811;
ppp. a VH comprising the amino acid sequence of SEQ ID NO:812, and a VL comprising the amino acid sequence of SEQ ID NO:813;
qqq. a VH comprising the amino acid sequence of SEQ ID NO:814, and a VL comprising the amino acid sequence of SEQ ID NO:815;
rrr. a VH comprising the amino acid sequence of SEQ ID NO:816, and a VL comprising the amino acid sequence of SEQ ID NO:817;
sss. a VH comprising the amino acid sequence of SEQ ID NO:818, and a VL comprising the amino acid sequence of SEQ ID NO:819;
ttt. a VH comprising the amino acid sequence of SEQ ID NO:820, and a VL comprising the amino acid sequence of SEQ ID NO:821;
uuu. a VH comprising the amino acid sequence of SEQ ID NO:822, and a VL comprising the amino acid sequence of SEQ ID NO:823;
vvv. a VH comprising the amino acid sequence of SEQ ID NO:824, and a VL comprising the amino acid sequence of SEQ ID NO:825;
www. a VH comprising the amino acid sequence of SEQ ID NO:826, and a VL comprising the amino acid sequence of SEQ ID NO:827;
xxx. a VH comprising the amino acid sequence of SEQ ID NO:828, and a VL comprising the amino acid sequence of SEQ ID NO:829;
yyy. a VH comprising the amino acid sequence of SEQ ID NO:830, and a VL comprising the amino acid sequence of SEQ ID NO:831;
zzz. a VH comprising the amino acid sequence of SEQ ID NO:832, and a VL comprising the amino acid sequence of SEQ ID NO:833;
aaaa. a VH comprising the amino acid sequence of SEQ ID NO:836, and a VL comprising the amino acid sequence of SEQ ID NO:837;
bbbb. a VH comprising the amino acid sequence of SEQ ID NO:838, and a VL comprising the amino acid sequence of SEQ ID NO:839;
cccc. a VH comprising the amino acid sequence of SEQ ID NO:840, and a VL comprising the amino acid sequence of SEQ ID NO:841;
dddd. a VH comprising the amino acid sequence of SEQ ID NO: 842, and a VL comprising the amino acid sequence of SEQ ID NO:843;
eeee. a VH comprising the amino acid sequence of SEQ ID NO:846, and a VL comprising the amino acid sequence of SEQ ID NO:847;
ffff. a VH comprising the amino acid sequence of SEQ ID NO:848, and a VL comprising the amino acid sequence of SEQ ID NO:849;
gggg. a VH comprising the amino acid sequence of SEQ ID NO:850, and a VL comprising the amino acid sequence of SEQ ID NO:851;
hhhh. a VH comprising the amino acid sequence of SEQ ID NO:852, and a VL comprising the amino acid sequence of SEQ ID NO:853;
iiii. a VH comprising the amino acid sequence of SEQ ID NO:854, and a VL comprising the amino acid sequence of SEQ ID NO:855;
jjjj. a VH comprising the amino acid sequence of SEQ ID NO:856, and a VL comprising the amino acid sequence of SEQ ID NO:857;
kkkk. a VH comprising the amino acid sequence of SEQ ID NO:858, and a VL comprising the amino acid sequence of SEQ ID NO:859;
llll. a VH comprising the amino acid sequence of SEQ ID NO:860, and a VL comprising the amino acid sequence of SEQ ID NO:861;
mmmm. a VH comprising the amino acid sequence of SEQ ID NO:862, and a VL comprising the amino acid sequence of SEQ ID NO:863;
nnnn. a VH comprising the amino acid sequence of SEQ ID NO:864, and a VL comprising the amino acid sequence of SEQ ID NO:865;
oooo. a VH comprising the amino acid sequence of SEQ ID NO:866, and a VL comprising the amino acid sequence of SEQ ID NO:867;
pppp. a VH comprising the amino acid sequence of SEQ ID NO:868, and a VL comprising the amino acid sequence of SEQ ID NO:869;
qqqq. a VH comprising the amino acid sequence of SEQ ID NO:870, and a VL comprising the amino acid sequence of SEQ ID NO:871;
rrrr. a VH comprising the amino acid sequence of SEQ ID NO:872, and a VL comprising the amino acid sequence of SEQ ID NO:873;
ssss. a VH comprising the amino acid sequence of SEQ ID NO:874, and a VL comprising the amino acid sequence of SEQ ID NO:875;
tttt. a VH comprising the amino acid sequence of SEQ ID NO:876, and a VL comprising the amino acid sequence of SEQ ID NO:877;
uuuu. a VH comprising the amino acid sequence of SEQ ID NO:878, and a VL comprising the amino acid sequence of SEQ ID NO:879;
vvvv. a VH comprising the amino acid sequence of SEQ ID NO:880, and a VL comprising the amino acid sequence of SEQ ID NO:881;
wwww. a VH comprising the amino acid sequence of SEQ ID NO:882, and a VL comprising the amino acid sequence of SEQ ID NO:883;
xxxx. a VH comprising the amino acid sequence of SEQ ID NO:884, and a VL comprising the amino acid sequence of SEQ ID NO:885;
yyyy. a VH comprising the amino acid sequence of SEQ ID NO:886, and a VL comprising the amino acid sequence of SEQ ID NO:887;
zzzz. a VH comprising the amino acid sequence of SEQ ID NO:888, and a VL comprising the amino acid sequence of SEQ ID NO:889; or
aaaaa. a VH comprising the amino acid sequence of SEQ ID NO:890, and a VL comprising the amino acid sequence of SEQ ID NO:891.

In some embodiments, the antibody or antigen bidnimng fragment thereof provided herein is genetically fused or chemically conjugated to an agent. The agent may be a detectable substance or a drug. The agent may be a functional label or a detectable label. In some embodiments, the antibody is genetically fused or chemically conjugated to an agent,
wherein optionally the agent a detectable substance or is a drug,
wherein optionally the detectable substance is selected from enzymes, prosthetic groups, fluorescent materials, luminescent materials, bioluminescent materials or chemiluminescent materials,
wherein optionally the enzymes are selected from horseradish peroxidase, alkaline phosphatase, beta-galactosidase, or acetylcholinesterase;
wherein optionally the prosthetic groups are selected from streptavidin/biotin or avidin/biotin;
wherein optionally the fluorescent materials are selected from umbelliferone, fluorescein, fluorescein isothiocynate, rhodamine, dichlorotriazinylamine fluorescein, dansyl chloride, or phycoerythrin;
wherein optionally the luminescent material is luminol;
wherein optionally the bioluminescent materials are selected from luciferase, luciferin, or aequorin; and
wherein optionally the chemiluminescent materials are selected from 225 Acγ-emitting, Auger-emitting, β-emitting, an alpha-emitting or positron-emitting radioactive isotope.

In some embodiments, the antibody provided herein is an IgG.

In some embodiments, the antibody provided herein is a humanized antibody. In some embodiments, the antibody provided herein is a humanized antibody or a fragment thereof, particularly an antigen binding fragment therof. In some embodiments, the antibody provided herein is a chimeric or chimerized antibody. In some embodiments, the antibody provided herein is a chimeric or chimerized antibody or a fragment thereof, particularly an antigen binding fragment thereof. In some embodiments, the antibody is in the form of an antibody F(ab′)2, scFv fragment, domain antibody, minibody, diabody, triabody or tetrabody.In some embodiments, the antibody provided herein is genetically fused or chemically conjugated to an agent. In some embodiments, the agent is a drug. In some embodiments, the agent a detectable substance. In some embodiments, the detectable substance is selected from enzymes, prosthetic groups, fluorescent materials, luminescent materials, bioluminescent materials or chemiluminescent materials. In some embodiments, the enzymes are selected from horseradish peroxidase, alkaline phosphatase, beta-galactosidase, or acetylcholinesterase. In some embodiments, the prosthetic groups are selected from streptavidin/biotin or avidin/biotin. In some embodiments, the fluorescent materials are selected from umbelliferone, fluorescein, fluorescein isothiocynate, rhodamine, dichlorotriazinylamine fluorescein, dansyl chloride, or phycoerythrin. In some embodiments, the luminescent material is luminol. In some embodiments, the bioluminescent materials are selected from luciferase, luciferin, or aequorin. In some embodiments, the chemiluminescent materials are selected from 225 Acγ-emitting, Auger-emitting, β-emitting, an alpha-emitting or positron-emitting radioactive isotope.

In another aspect, provided herein is a nucleic acid molecule encoding the antibody provided herein. In an embodiment, herein is provided nucleic acid molecule encoding any antibody or antigen binding fragment thereof as described herein. The nucleic acid may encode one antibody or fragment thereof. The nucleic acid may encode one or more or two or more antibodies or fragments thereof. The nucleic acid may encode a heavy chain or a light chain of an antibody. The nucleic acid may encode a heavy chain or a light chain of an antibody or fragment thereof. The nucleic acid may encode a heavy chain and a light chain of an antibody. The nucleic acid may encode a VHH antibody.

In another aspect, provided herein is a vector comprising the nucleic acid molecule provided herein.

In another aspect, provided herein is a host cell transformed with the vector provided herein.

Compositions comprising the antibody(ies) or active fragment(s) thereof are further provided, particularly further comprising a pharmaceutical excipient. In some embodiments, a composition is provided comprising the antibody or fragment thereof. In some embodiments, a composition is provided comprising the nucleic acid. In some embodiments, a composition is provided comprising a vector, and a pharmaceutically acceptable excipient. In another aspect, provided herein is a composition comprising the antibody provided herein, the nucleic acid molecule provided herein, or the vector provided herein, and a pharmaceutically acceptable excipient.

In another aspect, provided herein is a kit comprising the antibody provided herein.

In another aspect, provided herein is a method for detecting, selecting and/or enriching IL-23R in a sample comprising contacting the sample with the antibody provided herein. In some embodiments, the method comprises using the antibody provided herein in an immunohistochemistry (IHC) assay, an immunocytochemistry (ICC) assay, an immunoblotting assay, an immunoprecipitation assay, a flow cytometry assay, an ELISA, a radioimmunoassay, a mass spectrometry assay, or high throughput screening assay. In some embodiments, the method comprises using the antibody provided herein in a AbSeq/CITESeq assay. In some embodiments, the method comprises using the antibody provided herein in a proximity ligation assay. In some embodiments, method comprises using the antibody provided herein in an ELISA or other immunoassay. In some embodiments, the flow cytometry assay comprises three-steps of staining: (1) first, incubating the sample with the antibody provided herein; (2) second, incubating the sample with biotinylated anti-mouse IgG₂ₐ; (3) third, incubating the sample with streptavidin-PE. In some embodiments, to establish the background for the flow cytometry assay, cells incubated with buffer alone or the negative control antibody are used as a control group in step (1), and cells incubated without the biotinylated anti-mouse IgG₂ₐ secondary reagent are used as an additional control group in step (2).

In another aspect, provided herein is a method for detecting, selecting and/or enriching IL-23R in a sample comprising contacting the sample with an antibody that binds to the same epitope as the antibody provided herein, or an antibody that binds IL-23R competitively with the antibody provided herein.

In some embodiments, the method provided herein comprising contacting the sample with an antibody that binds to the same epitope and/or binds IL-23R competitively with the antibody comprising:
a. a VH comprising the amino acid sequence of SEQ ID NO:202 and a VL comprising the amino acid sequence of SEQ ID NO:203;
b. a VH comprising the amino acid sequence of SEQ ID NO:252 and a VL comprising the amino acid sequence of SEQ ID NO:253; or
c. a VH comprising the amino acid sequence of SEQ ID NO:156 and a VL comprising the amino acid sequence of SEQ ID NO:157.

In some embodiments, the method provided herein comprising contacting the sample with an antibody that binds to the same epitope and/or binds IL-23R competitively with the antibody comprising:
a. a VH comprising the amino acid sequence of SEQ ID NO:192 and a VL comprising the amino acid sequence of SEQ ID NO:193;
b. a VH comprising the amino acid sequence of SEQ ID NO:194 and a VL comprising the amino acid sequence of SEQ ID NO:195;
c. a VH comprising the amino acid sequence of SEQ ID NO:200 and a VL comprising the amino acid sequence of SEQ ID NO:201;
d. a VH comprising the amino acid sequence of SEQ ID NO:218 and a VL comprising the amino acid sequence of SEQ ID NO:219;
e. a VH comprising the amino acid sequence of SEQ ID NO:222 and a VL comprising the amino acid sequence of SEQ ID NO:223;
f. a VH comprising the amino acid sequence of SEQ ID NO:260 and a VL comprising the amino acid sequence of SEQ ID NO:261;
g. a VH comprising the amino acid sequence of SEQ ID NO:264 and a VL comprising the amino acid sequence of SEQ ID NO:265;
h. a VH comprising the amino acid sequence of SEQ ID NO:154 and a VL comprising the amino acid sequence of SEQ ID NO:155;
i. a VH comprising the amino acid sequence of SEQ ID NO:160 and a VL comprising the amino acid sequence of SEQ ID NO:161; or
j. a VH comprising the amino acid sequence of SEQ ID NO:164 and a VL comprising the amino acid sequence of SEQ ID NO:165.

In some embodiments, the method provided herein comprising contacting the sample with an antibody that binds to the same epitope and/or binds IL-23R competitively with the antibody comprising:
a. a VH comprising the amino acid sequence of SEQ ID NO:786 and a VL comprising the amino acid sequence of SEQ ID NO:787;
b. a VH comprising the amino acid sequence of SEQ ID NO:790 and a VL comprising the amino acid sequence of SEQ ID NO:791;
c. a VH comprising the amino acid sequence of SEQ ID NO:792 and a VL comprising the amino acid sequence of SEQ ID NO:793;
d. a VH comprising the amino acid sequence of SEQ ID NO:236 and a VL comprising the amino acid sequence of SEQ ID NO:237;
e. a VH comprising the amino acid sequence of SEQ ID NO:826 and a VL comprising the amino acid sequence of SEQ ID NO:827; or
f. a VH comprising the amino acid sequence of SEQ ID NO:828 and a VL comprising the amino acid sequence of SEQ ID NO:829.

In some embodiments, the method provided herein comprising contacting the sample with an antibody that binds to the same epitope and/or binds IL-23R competitively with the antibody comprising:
a. a VH comprising the amino acid sequence of SEQ ID NO:196 and a VL comprising the amino acid sequence of SEQ ID NO:197;
b. a VH comprising the amino acid sequence of SEQ ID NO:262 and a VL comprising the amino acid sequence of SEQ ID NO:263; or
c. a VH comprising the amino acid sequence of SEQ ID NO:152 and a VL comprising the amino acid sequence of SEQ ID NO:153.

In some embodiments, the method provided herein, comprising contacting the sample with an antibody that binds to the same epitope and/or binds IL-23R competitively with the antibody comprising: a VH comprising the amino acid sequence of SEQ ID NO:798 and a VL comprising the amino acid sequence of SEQ ID NO:799.

In some embodiments, the method provided herein comprising contacting the sample with an antibody that binds to the same epitope and/or binds IL-23R competitively with the antibody comprising:
a. a VH comprising the amino acid sequence of SEQ ID NO:206 and a VL comprising the amino acid sequence of SEQ ID NO:207;
b. a VH comprising the amino acid sequence of SEQ ID NO:250 and a VL comprising the amino acid sequence of SEQ ID NO:251;
c. a VH comprising the amino acid sequence of SEQ ID NO:144 and a VL comprising the amino acid sequence of SEQ ID NO:145; or
d. a VH comprising the amino acid sequence of SEQ ID NO:170 and a VL comprising the amino acid sequence of SEQ ID NO:171.

In some embodiments, the method provided herein comprising contacting the sample with an antibody that binds to the same epitope and/or binds IL-23R competitively with the antibody comprising: a VH comprising the amino acid sequence of SEQ ID NO:150 and a VL comprising the amino acid sequence of SEQ ID NO:151.

In some embodiments, the method provided herein comprising contacting the sample with an antibody that binds to the same epitope and/or binds IL-23R competitively with the antibody comprising:
a. a VH comprising the amino acid sequence of SEQ ID NO:198 and a VL comprising the amino acid sequence of SEQ ID NO:199; or
b. a VH comprising the amino acid sequence of SEQ ID NO:240 and a VL comprising the amino acid sequence of SEQ ID NO:241.

In some embodiments, the method provided herein comprising contacting the sample with an antibody that binds to the same epitope and/or binds IL-23R competitively with the antibody comprising:
a. a VH comprising the amino acid sequence of SEQ ID NO:204 and a VL comprising the amino acid sequence of SEQ ID NO:205; or
b. a VH comprising the amino acid sequence of SEQ ID NO:884 and a VL comprising the amino acid sequence of SEQ ID NO:885.

In some embodiments, the method provided herein is for detecting, selecting and/or enriching human IL-23R.

In some embodiments, the method provided herein is for detecting, selecting and/or enriching cells that express IL-23R.

In some embodiments, the method provided herein is for detecting denatured IL-23R.

In some embodiments, the method provided herein comprising contacting the sample with two or more antibodies each being the antibody provided herein. In some embodiments, the two or more antibodies bind to different epitopes of IL-23R. In some embodiments, the sample is from a human subject. In some embodiments, the human subject has a disease or disorder. In some embodiments, the disease or disorder is an IL-23R associated disease or disorder. In some embodiments, the human subject is a healthy human subject.

In another aspect, provided herein is a method of antagonizing or inhibiting IL-23R in a cell comprising contacting the cell with the antibody provided herein.

In another aspect, provided herein is a method of treating a disease or disorder in a subject, comprising administering to the subject the composition provided herein. In some embodiments, the disease or disorder is an IL-23R associated disease or disorder.

In an aspect, provided herein is a method of antagonizing or inhibiting IL-23R in a cell comprising contacting the cell with an antibody or antigen binding fragment thereof described herein.

In an aspect, provided herein is a method of treating a disease or disorder in a subject, comprising administering to the subject the composition hereof.

In accordance with the methods herein, the disease or disorder may an IL-23R associated disease or disorder. In some embodiments, the disease or disorder is associated with inflammatory, autoimmune inflammation diseases and/or related disorders. In some embodiments, the disease or disorder associated with inflammatory, autoimmune inflammation diseases and/or related disorders is selected from multiple sclerosis, asthma, rheumatoid arthritis, inflammation of the gut, inflammatory bowel diseases (IBDs), juvenile IBD, adolescent IBD, Crohn's disease, ulcerative colitis, Celiac disease (nontropical Sprue), microscopic colitis, collagenous colitis, eosinophilic gastroenteritis/esophagitis, colitis associated with radio- or chemo-therapy, colitis associated with disorders of innate immunity as in leukocyte adhesion deficiency-1, sarcoidosis, Systemic Lupus Erythematosus, ankylosing spondylitis (axial spondyloarthritis), psoriatic arthritis, psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, enteropathy associated with seronegative arthropathies, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

In some embodiments, the disease or disorder is associated with an autoimmune disease is selected from Ulcerative colitis (UC), Crohn's Disease (CD), psoriasis (PsO), or psoriatic arthritis (PsA).

### BRIEF DESCRIPTION OF THE FIGURES

FIG. 1 Representative polyclonal phage ELISA. Phage panning experiment XP37 showed enrichment of IL-23R binding starting at round 4 (R4).

FIG. 2 Representative monoclonal Fab ELISA for primary screening. Many monoclonal Fabs from XP37 round 5 (R5) show binding to IL-23R ECD but not to mouse IL-6. A mouse IL-6 binding Fab is used as a positive control for mouse IL-6 protein binding.

FIG. 3 IL-23 responsiveness of peripheral blood mononuclear cells (PBMCs) from 3 human donors. PBMCs from 3 donors were cultured on anti-CD3-coated flasks in serum-free medium supplemented with 100 ng/mL IL-1p. On day 4, cells were transferred to RPMI-1640 supplemented with 0.1% BSA for >4 hours and then stimulated with a serial titration of IL-23. Phosphorylated STAT3 was measured in cell lysates by Meso Scale Discovery (MSD). Each point represents the mean (±SD) of duplicate points.

FIG. 4 PBMC flow cytometry gating strategy and demonstration of lack of background binding with second- and third-step detection reagents. PBMCs were cultured on anti-CD3-coated flasks in serum-free medium supplemented with 100 ng/mL IL-13. On day 4, cells were stained with a viability dye in the presence of FcR blocking reagent and then incubated with 10 μg/mL anti-IL-23R or buffer alone for 60 minutes on ice. Cells were washed and incubated with biotinylated anti-mouse IgG₂ₐ or buffer alone for 30 minutes on ice. After a second wash, cells were incubated with SAV-PE and FITC-anti-human CD3, PE-Vio770-anti-human CD56, and APC-anti-human CD8. Cells were acquired on a FACSCanto cell analyzer, and data were analyzed using FlowJo software. (A) Gating strategy to identify live, single CD3+CD56⁻ T cells, CD3+CD56+ cells, and CD3⁻ CD56+ cells. PE vs CD8-APC in the absence of primary anti-IL-23R mAb (B), absence of primary anti-IL-23R mAb and secondary biotinylated anti-mouse IgG₂ₐ (C), or after staining with the negative control mAb, I23RB5, in the presence (D) or absence (E) of secondary biotinylated anti-mouse IgG₂ₐ.

FIG. 5 IL-23R detection by IL-23R mAbs from the same experiment shown in FIG. 4. The indicated phage display-derived anti-human IL-23R mAbs were tested and results are shown particularly for antibodies I23RB5, I23RB1, I23RB2, I23RB3, I23RB4 and I23RB7. IL-23R vs CD8 staining on gated CD3+CD56⁻ T cells (A), CD3+CD56+ cells (B), and CD3⁻ CD56+ cells (C).

FIG. 6 IL-23R detection by IL-23R mAbs on primary CD3+CD56⁻ T cells. IL-23R mAbs derived from immunizing mice with the human IL-23R extracellular domain were tested for flow cytometric detection of IL-23R expression by PBMCs stimulated for 4 days with anti-CD3 and IL-1β. IL-23R vs CD8 staining on gated CD3+CD56⁻ T cells is shown. (A) 123RB101 showed no binding over background staining. (B) I23RB42 and I23RB157 compete for IL-23R ECD protein binding and belong to epitope bin 1 (Table 11). I23RB157 showed no binding over background staining. (C) I23RB32, I23RB33, I23RB47, I23RB65, I23RB92, and I23RB94 belong to bin 2. (D) I23RB1, I23RB3, I23RB4, and I23RB76 belong to bin 3. (E) I23RB35 belongs to bin 4. (F) I23RB7 (and I23RB47 shown in part C) belong to bin 5. *Note that I23RB47 fell into bins 2 and 5.

FIG. 7 Expression knockdown by IL23R siRNA specifically reduces binding of IL-23R mAbs. PBMCs were cultured on anti-CD3-coated flasks in serum-free medium supplemented with 100 ng/mL IL-1β. On day 3, cells were electroporated with siRNA targeting the IL23R transcript or a negative control siRNA. After electroporation, the cells were cultured in serum-free medium on anti-CD3-coated flasks for an additional ˜24 hours. At that time, cells were pelleted for RNA isolation and real-time PCR analysis of IL23R and IL12RB1 transcript levels or stained for surface IL-23R and IL-12Rβ1. (A) Relative IL23R and IL12RB1 transcript levels in control and IL23R siRNA-electroporated PBMCs. (B) Gating strategy to identify live, single CD4+ and CD8+CD3+CD56⁻ T cells. (C) I23RB4, I23RB7, or I23RB76 (unfilled histograms) or I23RB5 (shaded histograms) staining on control (red) or IL23R (blue) siRNA-electroporated cells. (D) IL-12Rβ1 (unfilled histograms) or fluorescence minus one (FMO) (shaded histograms) staining on control (red) or IL23R (blue) siRNA-electroporated cells.

### DETAILED DESCRIPTION

The present disclosure is based in part on the surprisingly superior properties of the anti-IL-23R antibodies provided herein for detecting and/or measuring IL-23R in primary cells and native tissues as well as native and recombinant cell lines by various assays among other advantages.

In 2011, a proprietary anti-human IL-23R mAb was briefly described by Pidasheva et al. (PLoS One 6: e25038, 2011) in their study of the R381Q variant of the IL-23 receptor. Later, in 2017, Wines et al. (Immunol Cell Biol 95: 272-279, 2017) described three anti-human IL-23R monoclonal antibodies (mAbs) that, when combined in a single staining cocktail, were able to detect IL-23R on the surface of cultured Th17 and Th17/1 cell lines. Two of the mAbs additionally detected IL-23R on the surface of freshly isolated and cultured γδ T cells (Wines et al., 2017). Aside from these reagents, anti-IL-23R mAbs reported to detect IL-23R by flow cytometry are commercially available from R&D Systems (FAB14001, which is the same clone as MAB14001 but in labeled form) and abcam (ab222104). However, the experiments described in this application have shown that these commercial antibodies bind weakly to the IL-23R extracellular domain (ECD).

In this application, we describe the discovery and characterization of novel anti-human IL-23R antibodies validated for flow cytometric detection of IL-23R on the surface of primary human CD3+CD56⁻ T cells. Also, novel anti-IL-23R antibodies are described and characterized that bind human IL-23R and also rat IL-23R. These mAbs have a range of affinities, recognize multiple distinct epitopes on the IL-23R extracellular domain, and have the potential for use in applications beyond flow cytometry, including, but not limited to, receptor occupancy and target engagement assays, immunohistochemistry (IHC), Western blot, and immunoprecipitation with primary cells and native tissues as well as with native and recombinant cell lines. These mAbs have the potential for use in applications including monitoring or modulating IL-23R activity and expression, including in primary cells and native tissues. Further, the antibodies have applications in monitoring or modulating IL-23R mediated signaling in vitro, in vivo and ex vivo. Other applications involving IL-23R mediated signaling and response include in IL-23R mediated and associated diseases and conditions in an animal, including in animal models, in disease assessment, and in treatment or alleviation of IL-23R mediated or associated diseases.

### Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dubel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal antibodies, monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, antibodies including from Camelidae species (e.g., llama or alpaca) or their humanized variants, intrabodies, anti-idiotypic (anti-Id) antibodies, and functional fragments (e.g., antigen binding fragments) of any of the above, which refers to a portion of an antibody heavy or light chain polypeptide that retains some or all of the binding activity of the antibody from which the fragment was derived. Non-limiting examples of functional fragments (e.g., antigen binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment (antigen binding fragment) for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis or RIA. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (K_{D}) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise an antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1: 755-68 (2006)). Also available for the preparation of human monoclonal antibodies are methods described in Cole et al., Monoclonal Antibodies and Cancer Therapy 77 (1985); Boerner et al., J. Immunol. 147(1):86-95 (1991); and van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6(5):561-66 (1995); Brnggemann and Taussing, Curr. Opin. Biotechnol. 8(4):455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid isomerization or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a βsheet configuration, connected by three hypervariable regions, which form loops connecting, and in some cases form part of, the βsheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDR1, CDR2 and CDR3 in VH domain are also referred to as HCDR1, HCDR2 and HCDR3, respectively. CDR1, CDR2 and CDR3 in VL domain are also referred to as LCDR1, LCDR2 and LCDR3, respectively. Accordingly, CDRs are variable region sequences interspersed within the framework region sequences.

The term “gene expression” describes the conversion of the DNA gene sequence information into transcribed RNA (the initial unspliced RNA transcript or the mature mRNA) or the encoded protein product. Gene expression can be monitored by measuring the levels of either the entire RNA or protein products of the gene or their subsequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra; Nick Deschacht et al., J Immunol 2010; 184:5696-5704). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27(1):55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plnckthun, J. Mol. Biol. 309: 657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in Table 1 below.

TABLE 1
Exemplary CDRs According to Various Numbering Systems

| Loop | Kabat | AbM | Chothia | Contact | IMGT |
|---|---|---|---|---|---|
| CDR | L24--L34 | L24--L34 | L26--L32 | L30-- | L27--L38 |
| L1 | | | or | L36 | |
| | | | L24--L34 | | |
| CDR | L50--L56 | L50--L56 | L50--L52 | L46-- | L56--L65 |
| L2 | | | or | L55 | |
| | | | L50--L56 | | |
| CDR | L89--L97 | L89--L97 | L91--L96 | L89-- | L105-L117 |
| L3 | | | or | L96 | |
| | | | L89--L97 | | |
| CDR | H31--H35B | H26--H35B | H26--H32 | H30-- | H27--H38 |
| H1 | (Kabat | | . . . 34 | H35B | |
| | Numbering) | | | | |
| CDR | H31--H35 | H26--H35 | H26--H32 | H30-- | |
| H1 | (Chothia | | | H35 | |
| | Numbering) | | | | |
| CDR | H50--H65 | H50--H58 | H53--H55 | H47-- | H56--H65 |
| H2 | | | or | H58 | |
| | | | H52--H56 | | |
| CDR | H95--H102 | H95--H102 | H96--H101 | H93-- | H105-H117 |
| H3 | | | or | H101 | |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR1 as set forth in a specific VH” includes any CDR1 as defined by the exemplary CDR numbering systems described above, but is not limited thereby. Once a variable region (e.g., a VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “specificity” refers to selective recognition of an antigen binding protein for a particular epitope of an antigen. Natural antibodies, for example, are monospecific. The term “multispecific” as used herein denotes that an antigen binding protein has two or more antigen-binding sites of which at least two bind different antigens. “Bispecific” as used herein denotes that an antigen binding protein has two different antigen-binding specificities. The term “monospecific” antibody as used herein denotes an antigen binding protein that has one or more binding sites each of which bind the same antigen.

The term “valent” as used herein denotes the presence of a specified number of binding sites in an antigen binding protein. A natural antibody for example or a full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding an antibody described herein is a nucleic acid molecule that is identified and separated from at least one contaminant nucleic acid molecule with which it is ordinarily associated in the environment in which it was produced.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain a binding molecule (e.g., an antibody), for example, in isolated or purified form, together with a suitable amount of excipients.

The term “effective amount” or “therapeutically effective amount” as used herein refers to the amount of an antibody or a therapeutic molecule comprising an agent and the antibody or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., diabetes or a cancer).

“IL-23R associated disease or disorder” as used herein refers to a disease or disorder that comprises a cell or tissue in which IL-23R expression, activity or function is altered or abnormal, including a disease or disorder that: comprises a cell or tissue in with IL-23R is expressed or overexpressed; or comprises a cell on which IL-23R is abnormally expressed; or comprises a cell in or on which IL-23R is abnormally functioning.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### IL-23R Binding Molecules

Antibodies that Bind to IL-23R

In one aspect, provided herein are antibodies capable of binding to IL-23R. IL-23 is a disulfide-linked heterodimer of the IL-23p19 and IL-12/23p40 subunits. The receptor for IL-23 comprises the IL-23R and IL-12Rβ1 subunits. IL-23p19 binding to the N-terminal immunoglobulin (Ig)-like domain of IL-23R is followed by IL-12/23p40 binding to IL-12Rβ1. Ligand binding results in the phosphorylation of JAK2 and TYK2 followed by phosphorylation and nuclear translocation of STAT proteins. STAT3 plays a predominant role in mediating the biological effects of IL-23 signaling. In some embodiments, the antibodies provided herein bind to human IL-23R. An example of human IL-23R extracellular domain (ECD) sequence is shown in Table 3. In some embodiments, the antibodies provided herein bind to rat IL-23R. An example of human IL-23R extracellular domain (ECD) sequence is provided herein and in SEQ ID NO:1267. In some embodiments, the antibodies provided herein bind to human IL-23R and also bind to rat IL-23R. In some embodiments, the antibodies provided herein bind to human IL-23R ECD and rat IL-23R ECD. In some embodiments, the anti-IL-23R antibody provided herein modulates IL-23R activities. In some embodiments, the anti-IL-23R antibody provided herein is an antagonist antibody.

In some embodiments, the antibody or fragment thereof provided herein is an antagonist antibody against IL-23R. In some embodiments, the antibody or antigen binding fragment provided herein binds the target protein IL-23R and decreases the binding of IL-23 to IL-23R. In some embodiments, the antibody or antigen binding fragment provided herein binds the target protein IL-23R and decreases the binding of IL-23 to IL-23R to a basal level. In one aspect of this embodiment, the antibody or antigen binding fragment reduces the amount of IL-23 that binds to IL-23R. In a further aspect of this embodiment, the antibody or antigen binding fragment completely prevents IL-23 from binding to IL-23R. In a further embodiment, the antibody or antigen binding fragment inhibits STAT activation. In a further embodiment, the antibody or antigen binding fragment inhibits STAT3 phosphorylation. An antibody that inhibits one or more of these IL-23R functional properties (e.g., biochemical, immune, chemical, cellular, physiological or other biological activities, or the like) as determined according to methodologies known to the art and described herein, will be understood to relate to a statistically significant decrease or increase in the particular activity relative to that seen in the absence of the antibody (e.g., or when a control antibody of irrelevant specificity is present). In some embodiments, an antibody that inhibits IL-23R activity affects such a statistically significant decrease by at least 10% of the measured parameter, by at least 50%, 80% or 90%, and in certain embodiments an antibody of the disclosure may inhibit greater than 90%, 95%, 98% or 99% of an IL-23R activity.

In some embodiments, the anti-IL-23R antibody provided herein binds to IL-23R (e.g., human IL-23R) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In some embodiments, the anti-IL-23R antibody provided herein binds to IL-23R ECD (e.g., human IL-23R ECD) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻¹³ M to 10⁻¹³ M). In some embodiments, the anti-IL-23R antibody provided herein binds to human and rat IL-23R with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M. A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet@, using, for example, an Octet®Red96 system, or by Biacore®, using, for example, a Biacore®TM-2000 or a Biacore®TM-3000. An “on-rate” or “rate of association” or “association rate” or “kon” may also be determined with the same biolayer interferometry (BLI) or surface plasmon resonance (SPR) techniques described above using, for example, the Octet®Red96, the Biacore®TM-2000, the Biacore®TM-3000 system, the Biacore®TM-8K, or the Biacore®TM-8K+ system.

In one aspect, provided herein is an antibody that binds to IL-23R. In some embodiments, the antibody comprises a heavy chain variable region and a light chain variable region. In some embodiments, the IL-23R antibody is not a single domain antibody or nanobody. In some embodiments, the IL-23R antibody is a humanized antibody.

In certain embodiments, provided herein is an IL-23R antibody comprising a VH region, VL region, HCDR1, HCDR2, HCDR3, LCDR1, LCDR2, and/or LCDR3 of any one of the antibodies described herein. In some embodiments, provided herein is an IL-23R antibody comprising a VH region of any one of the antibodies described herein. In some embodiments, provided herein is an IL-23R antibody comprising a VL region of any one of the antibodies described herein. In some embodiments, provided herein is an IL-23R antibody comprising a VH region of any one of the antibodies described herein, and a VL region of any one of the antibodies described herein. In some embodiments, provided herein is an IL-23R antibody comprising a HCDR1, HCDR2, and HCDR3 of any one of the antibodies described herein. In some embodiments, provided herein is an IL-23R antibody comprising a LCDR1, LCDR2, and LCDR3 of any one of the antibodies described herein. In some embodiments, provided herein is an IL-23R antibody comprising a HCDR1, HCDR2, and HCDR3 of any one of the antibodies described herein; and a LCDR1, LCDR2, and LCDR3 of any one of the antibodies described herein. Representative VH and VL amino acid sequences, including HCDR1, HCDR2, HCDR3, LCDR1, LCDR2 and LCDR3 amino acid sequences, of IL-23R antibodies provided herein are provided in the Sequence Listing, as well as Tables 4-9.

In some embodiments, the antibody is a humanized antibody. In certain embodiments, the antibody is an IgG antibody. In other embodiments, the IgG antibody is an IgG1, IgG2, IgG3, or IgG4 antibody. In some embodiments, the antibody is a bispecific antibody. In certain embodiments, the antibody is multivalent. In other embodiments, the antibody is capable of binding at least three antigens. In some embodiments, the antibody is capable of binding at least five antigens.

In specific embodiments, the IL-23R antibody comprises a VH region and a VL region. In some embodiments, the IL-23R antibody is a single chain antibody. In some embodiments, the IL-23R antibody is a single domain antibody. In some embodiments, the IL-23R antibody is a nanobody. In certain embodiments, the IL-23R antibody is a VHH antibody. In certain embodiments, the IL-23R antibody is a llama antibody. In some embodiments, the IL-23R antibody is not a single chain antibody. In some embodiments, the IL-23R antibody is not a single domain antibody. In some embodiments, the IL-23R antibody is not a nanobody. In certain embodiments, the IL-23R antibody is not a VHH antibody. In certain embodiments, the IL-23R antibody is not a llama antibody. In some embodiments, the IL-23R antibody is a multispecific antibody. In other embodiments, the IL-23R is a bispecific antibody. In certain embodiments, the multispecific antibody comprises an antigen binding fragment of an IL-23R antibody provided herein. In other embodiments, the bispecific antibody comprises an antigen binding fragment of an IL-23R antibody provided herein.

In some embodiments, the anti-IL-23R antibodies provide herein are those described in the Examples below. Thus, in some embodiments, the antibody provided herein comprises one or more CDR sequences of any one of SEQ ID NOs:136-269, 786-899. In some embodiments, the antibody provided herein comprises the VH heavy chain CDRs (CDR1, CDR2 and CDR3) sequences of any one of SEQ ID NOs:136-269, 786-899. In some embodiments, the antibody provided herein comprises the VH heavy chain CDRs (CDR1, CDR2 and CDR3) sequences and the VL light chain CDRs (CDR1, CDR2 and CDR3) of any one of SEQ ID NOs:136-269, 786-899. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-IL-23R antibody is humanized. In some embodiments, the anti-IL-23R antibody comprises an acceptor human framework, e.g., a human immunoglobulin framework or a human consensus framework.

In some embodiments, the antibody or antigen binding fragment provided herein comprises an HCDR1, an HCDR2, and an HCDR3 as set forth in SEQ ID NO:138, and an LCDR1, an LCDR2, and an LCDR3 as set forth in SEQ ID NO:139. In some embodiments, the antibody or antigen binding fragment provided herein comprises an HCDR1, an HCDR2, and an HCDR3 as set forth in SEQ ID NO:142, and an LCDR1, an LCDR2, and an LCDR3 as set forth in SEQ ID NO: 143. In some embodiments, the antibody or antigen binding fragment provided herein comprises an HCDR1, an HCDR2, and an HCDR3 as set forth in SEQ ID NO:144, and an LCDR1, an LCDR2, and an LCDR3 as set forth in SEQ ID NO:145. In some embodiments, the antibody or antigen binding fragment provided herein comprises an HCDR1, an HCDR2, and an HCDR3 as set forth in SEQ ID NO:148, and an LCDR1, an LCDR2, and an LCDR3 as set forth in SEQ ID NO:149. In some embodiments, the antibody or antigen binding fragment provided herein comprises an HCDR1, an HCDR2, and an HCDR3 as set forth in SEQ ID NO:150, and an LCDR1, an LCDR2, and an LCDR3 as set forth in SEQ ID NO:151. In some embodiments, the antibody or antigen binding fragment provided herein comprises an HCDR1, an HCDR2, and an HCDR3 as set forth in SEQ ID NO: 152, and an LCDR1, an LCDR2, and an LCDR3 as set forth in SEQ ID NO:153. In some embodiments, the antibody or antigen binding fragment provided herein comprises an HCDR1, an HCDR2, and an HCDR3 as set forth in SEQ ID NO:154, and an LCDR1, an LCDR2, and an LCDR3 as set forth in SEQ ID NO:155. In some embodiments, the antibody or antigen binding fragment provided herein comprises an HCDR1, an HCDR2, and an HCDR3 as set forth in SEQ ID NO:156, and an LCDR1, an LCDR2, and an LCDR3 as set forth in SEQ ID NO:157. In some embodiments, the antibody or antigen binding fragment provided herein comprises an HCDR1, an HCDR2, and an HCDR3 as set forth in SEQ ID NO:158, and an LCDR1, an LCDR2, and an LCDR3 as set forth in SEQ ID NO: 159. In some embodiments, the antibody or antigen binding fragment provided herein comprises an HCDR1, an HCDR2, and an HCDR3 as set forth in SEQ ID NO:160, and an LCDR1, an LCDR2, and an LCDR3 as set forth in SEQ ID NO:161. In some embodiments, the antibody or antigen binding fragment provided herein comprises an HCDR1, an HCDR2, and an HCDR3 as set forth in SEQ ID NO:162, and an LCDR1, an LCDR2, and an LCDR3 as set forth in SEQ ID NO:163. In some embodiments, the antibody or antigen binding fragment provided herein comprises an HCDR1, an HCDR2, and an HCDR3 as set forth in SEQ ID NO:164, and an LCDR1, an LCDR2, and an LCDR3 as set forth in SEQ ID NO:165. In some embodiments, the antibody or antigen binding fragment provided herein comprises an HCDR1, an HCDR2, and an HCDR3 as set forth in SEQ ID NO: 166, and an LCDR1, an LCDR2, and an LCDR3 as set forth in SEQ ID NO:167. In some embodiments, the antibody or antigen binding fragment provided herein comprises an HCDR1, an HCDR2, and an HCDR3 as set forth in SEQ ID NO:168, and an LCDR1, an LCDR2, and an LCDR3 as set forth in SEQ ID NO:169. In some embodiments, the antibody or antigen binding fragment provided herein comprises an HCDR1, an HCDR2, and an HCDR3 as set forth in SEQ ID NO:170, and an LCDR1, an LCDR2, and an LCDR3 as set forth in SEQ ID NO:171. In some embodiments, the antibody or antigen binding fragment provided herein comprises an HCDR1, an HCDR2, and an HCDR3 as set forth in SEQ ID NO:172, and an LCDR1, an LCDR2, and an LCDR3 as set forth in SEQ ID NO: 173. In some embodiments, the antibody or antigen binding fragment provided herein comprises an HCDR1, an HCDR2, and an HCDR3 as set forth in SEQ ID NO:174, and an LCDR1, an LCDR2, and an LCDR3 as set forth in SEQ ID NO:175. In some embodiments, the antibody or antigen binding fragment provided herein comprises an HCDR1, an HCDR2, and an HCDR3 as set forth in SEQ ID NO:176, and an LCDR1, an LCDR2, and an LCDR3 as set forth in SEQ ID NO:177. In some embodiments, the antibody or antigen binding fragment provided herein comprises an HCDR1, an HCDR2, and an HCDR3 as set forth in SEQ ID NO:178, and an LCDR1, an LCDR2, and an LCDR3 as set forth in SEQ ID NO:179. In some embodiments, the antibody or antigen binding fragment provided herein comprises an HCDR1, an HCDR2, and an HCDR3 as set forth in SEQ ID NO: 180, and an LCDR1, an LCDR2, and an LCDR3 as set forth in SEQ ID NO:181. In some embodiments, the antibody or antigen binding fragment provided herein comprises an HCDR1, an HCDR2, and an HCDR3 as set forth in SEQ ID NO:182, and an LCDR1, an LCDR2, and an LCDR3 as set forth in SEQ ID NO:183. In some embodiments, the antibody or antigen binding fragment provided herein comprises an HCDR1, an HCDR2, and an HCDR3 as set forth in SEQ ID NO:186, and an LCDR1, an LCDR2, and an LCDR3 as set forth in SEQ ID NO:187. In some embodiments, the antibody or antigen binding fragment provided herein comprises an HCDR1, an HCDR2, and an HCDR3 as set forth in SEQ ID NO:188, and an LCDR1, an LCDR2, and an LCDR3 as set forth in SEQ ID NO: 189. In some embodiments, the antibody or antigen binding fragment provided herein comprises an HCDR1, an HCDR2, and an HCDR3 as set forth in SEQ ID NO:190, and an LCDR1, an LCDR2, and an LCDR3 as set forth in SEQ ID NO:191. In some embodiments, the antibody or antigen binding fragment provided herein comprises an HCDR1, an HCDR2, and an HCDR3 as set forth in SEQ ID NO:192, and an LCDR1, an LCDR2, and an LCDR3 as set forth in SEQ ID NO:193. In some embodiments, the antibody or antigen binding fragment provided herein comprises an HCDR1, an HCDR2, and an HCDR3 as set forth in SEQ ID NO:194, and an LCDR1, an LCDR2, and an LCDR3 as set forth in SEQ ID NO:195. In some embodiments, the antibody or antigen binding fragment provided herein comprises an HCDR1, an HCDR2, and an HCDR3 as set forth in SEQ ID NO: 196, and an LCDR1, an LCDR2, and an LCDR3 as set forth in SEQ ID NO:197. In some embodiments, the antibody or antigen binding fragment provided herein comprises an HCDR1, an HCDR2, and an HCDR3 as set forth in SEQ ID NO:198, and an LCDR1, an LCDR2, and an LCDR3 as set forth in SEQ ID NO:199. In some embodiments, the antibody or antigen binding fragment provided herein comprises an HCDR1, an HCDR2, and an HCDR3 as set forth in SEQ ID NO:200, and an LCDR1, an LCDR2, and an LCDR3 as set forth in SEQ ID NO:201. In some embodiments, the antibody or antigen binding fragment provided herein comprises an HCDR1, an HCDR2, and an HCDR3 as set forth in SEQ ID NO:202, and an LCDR1, an LCDR2, and an LCDR3 as set forth in SEQ ID NO:203. In some embodiments, the antibody or antigen binding fragment provided herein comprises an HCDR1, an HCDR2, and an HCDR3 as set forth in SEQ ID NO:204, and an LCDR1, an LCDR2, and an LCDR3 as set forth in SEQ ID NO:205. In some embodiments, the antibody or antigen binding fragment provided herein comprises an HCDR1, an HCDR2, and an HCDR3 as set forth in SEQ ID NO:206, and an LCDR1, an LCDR2, and an LCDR3 as set forth in SEQ ID NO:207. In some embodiments, the antibody or antigen binding fragment provided herein comprises an HCDR1, an HCDR2, and an HCDR3 as set forth in SEQ ID NO:208, and an LCDR1, an LCDR2, and an LCDR3 as set forth in SEQ ID NO:209. In some embodiments, the antibody or antigen binding fragment provided herein comprises an HCDR1, an HCDR2, and an HCDR3 as set forth in SEQ ID NO:210, and an LCDR1, an LCDR2, and an LCDR3 as set forth in SEQ ID NO:211. In some embodiments, the antibody or antigen binding fragment provided herein comprises an HCDR1, an HCDR2, and an HCDR3 as set forth in SEQ ID NO:212, and an LCDR1, an LCDR2, and an LCDR3 as set forth in SEQ ID NO:213. In some embodiments, the antibody or antigen binding fragment provided herein comprises an HCDR1, an HCDR2, and an HCDR3 as set forth in SEQ ID NO:214, and an LCDR1, an LCDR2, and an LCDR3 as set forth in SEQ ID NO:215. In some embodiments, the antibody or antigen binding fragment provided herein comprises an HCDR1, an HCDR2, and an HCDR3 as set forth in SEQ ID NO:216, and an LCDR1, an LCDR2, and an LCDR3 as set forth in SEQ ID NO:217. In some embodiments, the antibody or antigen binding fragment provided herein comprises an HCDR1, an HCDR2, and an HCDR3 as set forth in SEQ ID NO:218, and an LCDR1, an LCDR2, and an LCDR3 as set forth in SEQ ID NO:219. In some embodiments, the antibody or antigen binding fragment provided herein comprises an HCDR1, an HCDR2, and an HCDR3 as set forth in SEQ ID NO:220, and an LCDR1, an LCDR2, and an LCDR3 as set forth in SEQ ID NO:221. In some embodiments, the antibody or antigen binding fragment provided herein comprises an HCDR1, an HCDR2, and an HCDR3 as set forth in SEQ ID NO:222, and an LCDR1, an LCDR2, and an LCDR3 as set forth in SEQ ID NO:223. In some embodiments, the antibody or antigen binding fragment provided herein comprises an HCDR1, an HCDR2, and an HCDR3 as set forth in SEQ ID NO:226, and an LCDR1, an LCDR2, and an LCDR3 as set forth in SEQ ID NO:227. In some embodiments, the antibody or antigen binding fragment provided herein comprises an HCDR1, an HCDR2, and an HCDR3 as set forth in SEQ ID NO:228, and an LCDR1, an LCDR2, and an LCDR3 as set forth in SEQ ID NO:229. In some embodiments, the antibody or antigen binding fragment provided herein comprises an HCDR1, an HCDR2, and an HCDR3 as set forth in SEQ ID NO:230, and an LCDR1, an LCDR2, and an LCDR3 as set forth in SEQ ID NO:231. In some embodiments, the antibody or antigen binding fragment provided herein comprises an HCDR1, an HCDR2, and an HCDR3 as set forth in SEQ ID NO:232, and an LCDR1, an LCDR2, and an LCDR3 as set forth in SEQ ID NO:233. In some embodiments, the antibody or antigen binding fragment provided herein comprises an HCDR1, an HCDR2, and an HCDR3 as set forth in SEQ ID NO:234, and an LCDR1, an LCDR2, and an LCDR3 as set forth in SEQ ID NO:235. In some embodiments, the antibody or antigen binding fragment provided herein comprises an HCDR1, an HCDR2, and an HCDR3 as set forth in SEQ ID NO:236, and an LCDR1, an LCDR2, and an LCDR3 as set forth in SEQ ID NO:237. In some embodiments, the antibody or antigen binding fragment provided herein comprises an HCDR1, an HCDR2, and an HCDR3 as set forth in SEQ ID NO:238, and an LCDR1, an LCDR2, and an LCDR3 as set forth in SEQ ID NO:239. In some embodiments, the antibody or antigen binding fragment provided herein comprises an HCDR1, an HCDR2, and an HCDR3 as set forth in SEQ ID NO:240, and an LCDR1, an LCDR2, and an LCDR3 as set forth in SEQ ID NO:241. In some embodiments, the antibody or antigen binding fragment provided herein comprises an HCDR1, an HCDR2, and an HCDR3 as set forth in SEQ ID NO:246, and an LCDR1, an LCDR2, and an LCDR3 as set forth in SEQ ID NO:247. In some embodiments, the antibody or antigen binding fragment provided herein comprises an HCDR1, an HCDR2, and an HCDR3 as set forth in SEQ ID NO:248, and an LCDR1, an LCDR2, and an LCDR3 as set forth in SEQ ID NO:249. In some embodiments, the antibody or antigen binding fragment provided herein comprises an HCDR1, an HCDR2, and an HCDR3 as set forth in SEQ ID NO:250, and an LCDR1, an LCDR2, and an LCDR3 as set forth in SEQ ID NO:251. In some embodiments, the antibody or antigen binding fragment provided herein comprises an HCDR1, an HCDR2, and an HCDR3 as set forth in SEQ ID NO:252, and an LCDR1, an LCDR2, and an LCDR3 as set forth in SEQ ID NO:253. In some embodiments, the antibody or antigen binding fragment provided herein comprises an HCDR1, an HCDR2, and an HCDR3 as set forth in SEQ ID NO:254, and an LCDR1, an LCDR2, and an LCDR3 as set forth in SEQ ID NO:255. In some embodiments, the antibody or antigen binding fragment provided herein comprises an HCDR1, an HCDR2, and an HCDR3 as set forth in SEQ ID NO:256, and an LCDR1, an LCDR2, and an LCDR3 as set forth in SEQ ID NO:257. In some embodiments, the antibody or antigen binding fragment provided herein comprises an HCDR1, an HCDR2, and an HCDR3 as set forth in SEQ ID NO:258, and an LCDR1, an LCDR2, and an LCDR3 as set forth in SEQ ID NO:259. In some embodiments, the antibody or antigen binding fragment provided herein comprises an HCDR1, an HCDR2, and an HCDR3 as set forth in SEQ ID NO:260, and an LCDR1, an LCDR2, and an LCDR3 as set forth in SEQ ID NO:261. In some embodiments, the antibody or antigen binding fragment provided herein comprises an HCDR1, an HCDR2, and an HCDR3 as set forth in SEQ ID NO:262, and a LCDR1, an LCDR2, and an LCDR3 as set forth in SEQ ID NO:263. In some embodiments, the antibody or antigen binding fragment provided herein comprises an HCDR1, an HCDR2, and an HCDR3 as set forth in SEQ ID NO:264, and an LCDR1, an LCDR2, and an LCDR3 as set forth in SEQ ID NO:265. In some embodiments, the antibody or antigen binding fragment provided herein comprises an HCDR1, an HCDR2, and an HCDR3 as set forth in SEQ ID NO:268, and an LCDR1, an LCDR2, and an LCDR3 as set forth in SEQ ID NO:269. In some embodiments, the antibody or antigen binding fragment provided herein comprises an HCDR1, an HCDR2, and an HCDR3 as set forth in SEQ ID NO:786, and an LCDR1, an LCDR2, and an LCDR3 as set forth in SEQ ID NO:787. In some embodiments, the antibody or antigen binding fragment provided herein comprises an HCDR1, an HCDR2, and an HCDR3 as set forth in SEQ ID NO:788, and an LCDR1, an LCDR2, and an LCDR3 as set forth in SEQ ID NO:789. In some embodiments, the antibody or antigen binding fragment provided herein comprises an HCDR1, an HCDR2, and an HCDR3 as set forth in SEQ ID NO:790, and an LCDR1, an LCDR2, and an LCDR3 as set forth in SEQ ID NO:791. In some embodiments, the antibody or antigen binding fragment provided herein comprises an HCDR1, an HCDR2, and an HCDR3 as set forth in SEQ ID NO:792, and an LCDR1, an LCDR2, and an LCDR3 as set forth in SEQ ID NO:793. In some embodiments, the antibody or antigen binding fragment provided herein comprises an HCDR1, an HCDR2, and an HCDR3 as set forth in SEQ ID NO:798, and an LCDR1, an LCDR2, and an LCDR3 as set forth in SEQ ID NO:799. In some embodiments, the antibody or antigen binding fragment provided herein comprises an HCDR1, an HCDR2, and an HCDR3 as set forth in SEQ ID NO:806, and an LCDR1, an LCDR2, and an LCDR3 as set forth in SEQ ID NO:807. In some embodiments, the antibody or antigen binding fragment provided herein comprises an HCDR1, an HCDR2, and an HCDR3 as set forth in SEQ ID NO:808, and an LCDR1, an LCDR2, and an LCDR3 as set forth in SEQ ID NO:809. In some embodiments, the antibody or antigen binding fragment provided herein comprises an HCDR1, an HCDR2, and an HCDR3 as set forth in SEQ ID NO:810, and an LCDR1, an LCDR2, and an LCDR3 as set forth in SEQ ID NO:811. In some embodiments, the antibody or antigen binding fragment provided herein comprises an HCDR1, an HCDR2, and an HCDR3 as set forth in SEQ ID NO:812, and an LCDR1, an LCDR2, and an LCDR3 as set forth in SEQ ID NO:813. In some embodiments, the antibody or antigen binding fragment provided herein comprises an HCDR1, an HCDR2, and an HCDR3 as set forth in SEQ ID NO:814, and an LCDR1, an LCDR2, and an LCDR3 as set forth in SEQ ID NO:815. In some embodiments, the antibody or antigen binding fragment provided herein comprises an HCDR1, an HCDR2, and an HCDR3 as set forth in SEQ ID NO:816, and an LCDR1, an LCDR2, and an LCDR3 as set forth in SEQ ID NO:817. In some embodiments, the antibody or antigen binding fragment provided herein comprises an HCDR1, an HCDR2, and an HCDR3 as set forth in SEQ ID NO:818, and an LCDR1, an LCDR2, and an LCDR3 as set forth in SEQ ID NO:819. In some embodiments, the antibody or antigen binding fragment provided herein comprises an HCDR1, an HCDR2, and an HCDR3 as set forth in SEQ ID NO:820, and an LCDR1, an LCDR2, and an LCDR3 as set forth in SEQ ID NO:821. In some embodiments, the antibody or antigen binding fragment provided herein comprises an HCDR1, an HCDR2, and an HCDR3 as set forth in SEQ ID NO:822, and an LCDR1, an LCDR2, and an LCDR3 as set forth in SEQ ID NO:823. In some embodiments, the antibody or antigen binding fragment provided herein comprises an HCDR1, an HCDR2, and an HCDR3 as set forth in SEQ ID NO:824, and an LCDR1, an LCDR2, and an LCDR3 as set forth in SEQ ID NO:825. In some embodiments, the antibody or antigen binding fragment provided herein comprises an HCDR1, an HCDR2, and an HCDR3 as set forth in SEQ ID NO:826, and an LCDR1, an LCDR2, and an LCDR3 as set forth in SEQ ID NO:827. In some embodiments, the antibody or antigen binding fragment provided herein comprises an HCDR1, an HCDR2, and an HCDR3 as set forth in SEQ ID NO:828, and an LCDR1, an LCDR2, and an LCDR3 as set forth in SEQ ID NO:829. In some embodiments, the antibody or antigen binding fragment provided herein comprises an HCDR1, an HCDR2, and an HCDR3 as set forth in SEQ ID NO:830, and an LCDR1, an LCDR2, and an LCDR3 as set forth in SEQ ID NO:831. In some embodiments, the antibody or antigen binding fragment provided herein comprises an HCDR1, an HCDR2, and an HCDR3 as set forth in SEQ ID NO:832, and an LCDR1, an LCDR2, and an LCDR3 as set forth in SEQ ID NO:833. In some embodiments, the antibody or antigen binding fragment provided herein comprises an HCDR1, an HCDR2, and an HCDR3 as set forth in SEQ ID NO:836, and an LCDR1, an LCDR2, and an LCDR3 as set forth in SEQ ID NO:837. In some embodiments, the antibody or antigen binding fragment provided herein comprises an HCDR1, an HCDR2, and an HCDR3 as set forth in SEQ ID NO:838, and an LCDR1, an LCDR2, and an LCDR3 as set forth in SEQ ID NO:839. In some embodiments, the antibody or antigen binding fragment provided herein comprises an HCDR1, an HCDR2, and an HCDR3 as set forth in SEQ ID NO:840, and an LCDR1, an LCDR2, and an LCDR3 as set forth in SEQ ID NO:841. In some embodiments, the antibody or antigen binding fragment provided herein comprises an HCDR1, an HCDR2, and an HCDR3 as set forth in SEQ ID NO: 842, and an LCDR1, an LCDR2, and an LCDR3 as set forth in SEQ ID NO:843. In some embodiments, the antibody or antigen binding fragment provided herein comprises an HCDR1, an HCDR2, and an HCDR3 as set forth in SEQ ID NO:846, and an LCDR1, an LCDR2, and an LCDR3 as set forth in SEQ ID NO:847. In some embodiments, the antibody or antigen binding fragment provided herein comprises an HCDR1, an HCDR2, and an HCDR3 as set forth in SEQ ID NO:848, and an LCDR1, an LCDR2, and an LCDR3 as set forth in SEQ ID NO:849. In some embodiments, the antibody or antigen binding fragment provided herein comprises an HCDR1, an HCDR2, and an HCDR3 as set forth in SEQ ID NO:850, and an LCDR1, an LCDR2, and an LCDR3 as set forth in SEQ ID NO:851. In some embodiments, the antibody or antigen binding fragment provided herein comprises an HCDR1, an HCDR2, and an HCDR3 as set forth in SEQ ID NO:852, and an LCDR1, an LCDR2, and an LCDR3 as set forth in SEQ ID NO:853. In some embodiments, the antibody or antigen binding fragment provided herein comprises an HCDR1, an HCDR2, and an HCDR3 as set forth in SEQ ID NO:854, and an LCDR1, an LCDR2, and an LCDR3 as set forth in SEQ ID NO:855. In some embodiments, the antibody or antigen binding fragment provided herein comprises an HCDR1, an HCDR2, and an HCDR3 as set forth in SEQ ID NO:856, and an LCDR1, an LCDR2, and an LCDR3 as set forth in SEQ ID NO:857. In some embodiments, the antibody or antigen binding fragment provided herein comprises an HCDR1, an HCDR2, and an HCDR3 as set forth in SEQ ID NO:858, and an LCDR1, an LCDR2, and an LCDR3 as set forth in SEQ ID NO:859. In some embodiments, the antibody or antigen binding fragment provided herein comprises an HCDR1, an HCDR2, and an HCDR3 as set forth in SEQ ID NO:860, and an LCDR1, an LCDR2, and an LCDR3 as set forth in SEQ ID NO:861. In some embodiments, the antibody or antigen binding fragment provided herein comprises an HCDR1, an HCDR2, and an HCDR3 as set forth in SEQ ID NO:862, and an LCDR1, an LCDR2, and an LCDR3 as set forth in SEQ ID NO:863. In some embodiments, the antibody or antigen binding fragment provided herein comprises an HCDR1, an HCDR2, and an HCDR3 as set forth in SEQ ID NO:864, and an LCDR1, an LCDR2, and an LCDR3 as set forth in SEQ ID NO:865. In some embodiments, the antibody or antigen binding fragment provided herein comprises an HCDR1, an HCDR2, and an HCDR3 as set forth in SEQ ID NO:866, and an LCDR1, an LCDR2, and an LCDR3 as set forth in SEQ ID NO:867. In some embodiments, the antibody or antigen binding fragment provided herein comprises an HCDR1, an HCDR2, and an HCDR3 as set forth in SEQ ID NO:868, and an LCDR1, an LCDR2, and an LCDR3 as set forth in SEQ ID NO:869. In some embodiments, the antibody or antigen binding fragment provided herein comprises an HCDR1, an HCDR2, and an HCDR3 as set forth in SEQ ID NO:870, and an LCDR1, an LCDR2, and an LCDR3 as set forth in SEQ ID NO:871. In some embodiments, the antibody or antigen binding fragment provided herein comprises an HCDR1, an HCDR2, and an HCDR3 as set forth in SEQ ID NO:872, and an LCDR1, an LCDR2, and an LCDR3 as set forth in SEQ ID NO:873. In some embodiments, the antibody or antigen binding fragment provided herein comprises an HCDR1, an HCDR2, and an HCDR3 as set forth in SEQ ID NO:874, and an LCDR1, an LCDR2, and an LCDR3 as set forth in SEQ ID NO:875. In some embodiments, the antibody or antigen binding fragment provided herein comprises an HCDR1, an HCDR2, and an HCDR3 as set forth in SEQ ID NO:876, and an LCDR1, an LCDR2, and an LCDR3 as set forth in SEQ ID NO:877. In some embodiments, the antibody or antigen binding fragment provided herein comprises an HCDR1, an HCDR2, and an HCDR3 as set forth in SEQ ID NO:878, and an LCDR1, an LCDR2, and an LCDR3 as set forth in SEQ ID NO:879. In some embodiments, the antibody or antigen binding fragment provided herein comprises an HCDR1, an HCDR2, and an HCDR3 as set forth in SEQ ID NO:880, and an LCDR1, an LCDR2, and an LCDR3 as set forth in SEQ ID NO:881. In some embodiments, the antibody or antigen binding fragment provided herein comprises an HCDR1, an HCDR2, and an HCDR3 as set forth in SEQ ID NO:882, and an LCDR1, an LCDR2, and an LCDR3 as set forth in SEQ ID NO:883. In some embodiments, the antibody or antigen binding fragment provided herein comprises an HCDR1, an HCDR2, and an HCDR3 as set forth in SEQ ID NO:884, and an LCDR1, an LCDR2, and an LCDR3 as set forth in SEQ ID NO:885. In some embodiments, the antibody or antigen binding fragment provided herein comprises an HCDR1, an HCDR2, and an HCDR3 as set forth in SEQ ID NO:886, and an LCDR1, an LCDR2, and an LCDR3 as set forth in SEQ ID NO:887. In some embodiments, the antibody or antigen binding fragment provided herein comprises an HCDR1, an HCDR2, and an HCDR3 as set forth in SEQ ID NO:888, and an LCDR1, an LCDR2, and an LCDR3 as set forth in SEQ ID NO:889. In some embodiments, the antibody or antigen binding fragment provided herein comprises an HCDR1, an HCDR2, and an HCDR3 as set forth in SEQ ID NO:890, and an LCDR1, an LCDR2, and an LCDR3 as set forth in SEQ ID NO:891. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In other embodiments, provided herein is an antibody that binds to IL-23R comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 276, 288, 294, 306, 312, 318, 324, 330, 336, 342, 348, 354, 360, 366, 372, 378, 384, 390, 396, 402, 408, 420, 426, 432, 438, 444, 450, 456, 462, 468, 474, 480, 486, 492, 498, 504, 510, 516, 522, 528, 540, 546, 552, 558, 564, 570, 576, 582, 600, 606, 612, 618, 624, 630, 636, 642, 648, 654, 666, 900, 906, 912, 918, 936, 960, 966, 972, 978, 984, 990, 996, 1002, 1008, 1014, 1020, 1026, 1032, 1038, 1050, 1056, 1062, 1068, 1080, 1086, 1092, 1098, 1104, 1110, 1116, 1122, 1128, 1134, 1140, 1146, 1152, 1158, 1164, 1170, 1176, 1182, 1188, 1194, 1200, 1206 and 1212; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 277, 289, 295, 307, 313, 319, 325, 331, 337, 343, 349, 355, 361, 367, 373, 379, 385, 391, 397, 403, 409, 421, 427, 433, 439, 445, 451, 457, 463, 469, 475, 481, 487, 493, 499, 505, 511, 517, 523, 529, 541, 547, 553, 559, 565, 571, 577, 583, 601, 607, 613, 619, 625, 631, 637, 643, 649, 655, 667, 901, 907, 913, 919, 937, 961, 967, 973, 979, 985, 991, 997, 1003, 1009, 1015, 1021, 1027, 1033, 1039, 1051, 1057, 1063, 1069, 1081, 1087, 1093, 1099, 1105, 1111, 1117, 1123, 1129, 1135, 1141, 1147, 1153, 1159, 1165, 1171, 1177, 1183, 1189, 1195, 1201, 1207 and 1213, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 278, 290, 296, 308, 314, 320, 326, 332, 338, 344, 350, 356, 362, 368, 374, 380, 386, 392, 398, 404, 410, 422, 428, 434, 440, 446, 452, 458, 464, 470, 476, 482, 488, 494, 500, 506, 512, 518, 524, 530, 542, 548, 554, 560, 566, 572, 578, 584, 602, 608, 614, 620, 626, 632, 638, 644, 650, 656, 668, 902, 908, 914, 920, 938, 962, 968, 974, 980, 986, 992, 998, 1004, 1010, 1016, 1022, 1028, 1034, 1040, 1052, 1058, 1064, 1070, 1082, 1088, 1094, 1100, 1106, 1112, 1118, 1124, 1130, 1136, 1142, 1148, 1154, 1160, 1166, 1172, 1178, 1184, 1190, 1196, 1202, 1208 and 1214; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 279, 291, 297, 309, 315, 321, 327, 333, 339, 345, 351, 357, 363, 369, 375, 381, 387, 393, 399, 405, 411, 423, 429, 435, 441, 447, 453, 459, 465, 471, 477, 483, 489, 495, 501, 507, 513, 519, 525, 531, 543, 549, 555, 561, 567, 573, 579, 585, 603, 609, 615, 621, 627, 633, 639, 645, 651, 657, 669, 903, 909, 915, 921, 939, 963, 969, 975, 981, 987, 993, 999, 1005, 1011, 1017, 1023, 1029, 1035, 1041, 1053, 1059, 1065, 1071, 1083, 1089, 1095, 1101, 1107, 1113, 1119, 1125, 1131, 1137, 1143, 1149, 1155, 1161, 1167, 1173, 1179, 1185, 1191, 1197, 1203, 1209 and 1215; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 280, 292, 298, 310, 316, 322, 328, 334, 340, 346, 352, 358, 364, 370, 376, 382, 388, 394, 400, 406, 412, 424, 430, 436, 442, 448, 454, 460, 466, 472, 478, 484, 490, 496, 502, 508, 514, 520, 526, 532, 544, 550, 556, 562, 568, 574, 580, 586, , 604, 610, 616, 622, 628, 634, 640, 646, 652, 658, 670, 904, 910, 916, 922, 940, 964, 970, 976, 982, 988, 994, 1000, 1006, 1012, 1018, 1024, 1030, 1036, 1042, 1054, 1060, 1066, 1072, 1084, 1090, 1096, 1102, 1108, 1114, 1120, 1126, 1132, 1138, 1144, 1150, 1156, 1162, 1168, 1174, 1180, 1186, 1192, 1198, 1204, 1210 and 1216; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to any of SEQ ID NOs: 281, 293, 299, 311, 317, 323, 329, 335, 341, 347, 353, 359, 365, 371, 377, 383, 389, 395, 401, 407, 413, 425, 431, 437, 443, 449, 455, 461, 467, 473, 479, 485, 491, 497, 503, 509, 515, 521, 527, 533, 545, 551, 557, 563, 569, 575, 581, 587, 605, 611, 617, 623, 629, 635, 641, 647, 653, 659, 671, 905, 911, 917, 923, 941, 965, 971, 977, 983, 989, 995, 1001, 1007, 1013, 1019, 1025, 1031, 1037, 1043, 1055, 1061, 1067, 1073, 1085, 1091, 1097, 1103, 1109, 1115, 1121, 1127, 1133, 1139, 1145, 1151, 1157, 1163, 1169, 1175, 1181, 1187, 1193, 1199, 1205, 1211 and 1217. In some embodiments, the anti-IL-23R antibody is humanized. In some embodiments, the anti-IL-23R antibody comprises an acceptor human framework, e.g., a human immunoglobulin framework or a human consensus framework.

In some specific embodiments, provided herein is an antibody that binds to IL-23R, comprising: (i) a VH comprising an HCDR1, an HCDR2, and an HCDR3 having an amino acid sequence of SEQ ID NOs:276, 277, and 278, respectively, and (ii) an VL comprising an LCDR1, an LCDR2, and an LCDR3 having an amino acid sequence of SEQ ID NOs:279, 280, and 281, respectively. In some specific embodiments, provided herein is an antibody that binds to IL-23R, comprising: (i) a VH comprising an HCDR1, an HCDR2, and an HCDR3 having an amino acid sequence of SEQ ID NOs:288, 289, and 290, respectively, and (ii) an VL comprising an LCDR1, an LCDR2, and an LCDR3 having an amino acid sequence of SEQ ID NOs:291, 292, and 293, respectively. In some specific embodiments, provided herein is an antibody that binds to IL-23R, comprising: (i) a VH comprising an HCDR1, an HCDR2, and an HCDR3 having an amino acid sequence of SEQ ID NOs:294, 295, and 296, respectively, and (ii) an VL comprising an LCDR1, an LCDR2, and an LCDR3 having an amino acid sequence of SEQ ID NOs:297, 298, and 299, respectively. In some specific embodiments, provided herein is an antibody that binds to IL-23R, comprising: (i) a VH comprising an HCDR1, an HCDR2, and an HCDR3 having an amino acid sequence of SEQ ID NOs:306, 307, and 308, respectively, and (ii) an VL comprising an LCDR1, an LCDR2, and an LCDR3 having an amino acid sequence of SEQ ID NOs:309, 310, and 311, respectively. In some specific embodiments, provided herein is an antibody that binds to IL-23R, comprising: (i) a VH comprising an HCDR1, an HCDR2, and an HCDR3 having an amino acid sequence of SEQ ID NOs:312, 313, and 314, respectively, and (ii) an VL comprising an LCDR1, an LCDR2, and an LCDR3 having an amino acid sequence of SEQ ID NOs:315, 316, and 317, respectively. In some specific embodiments, provided herein is an antibody that binds to IL-23R, comprising: (i) a VH comprising an HCDR1, an HCDR2, and an HCDR3 having an amino acid sequence of SEQ ID NOs:318, 319, and 320, respectively, and (ii) an VL comprising an LCDR1, an LCDR2, and an LCDR3 having an amino acid sequence of SEQ ID NOs:321, 322, and 323, respectively. In some specific embodiments, provided herein is an antibody that binds to IL-23R, comprising: (i) a VH comprising an HCDR1, an HCDR2, and an HCDR3 having an amino acid sequence of SEQ ID NOs:324, 325, and 326, respectively, and (ii) an VL comprising an LCDR1, an LCDR2, and an LCDR3 having an amino acid sequence of SEQ ID NOs:327, 328, and 329, respectively. In some specific embodiments, provided herein is an antibody that binds to IL-23R, comprising: (i) a VH comprising an HCDR1, an HCDR2, and an HCDR3 having an amino acid sequence of SEQ ID NOs:330, 331, and 332, respectively, and (ii) an VL comprising an LCDR1, an LCDR2, and an LCDR3 having an amino acid sequence of SEQ ID NOs:333, 334, and 335, respectively. In some specific embodiments, provided herein is an antibody that binds to IL-23R, comprising: (i) a VH comprising an HCDR1, an HCDR2, and an HCDR3 having an amino acid sequence of SEQ ID NOs:336, 337, and 338, respectively, and (ii) an VL comprising an LCDR1, an LCDR2, and an LCDR3 having an amino acid sequence of SEQ ID NOs:339, 340, and 341, respectively. In some specific embodiments, provided herein is an antibody that binds to IL-23R, comprising: (i) a VH comprising an HCDR1, an HCDR2, and an HCDR3 having an amino acid sequence of SEQ ID NOs:342, 343, and 344, respectively, and (ii) an VL comprising an LCDR1, an LCDR2, and an LCDR3 having an amino acid sequence of SEQ ID NOs:345, 346, and 347, respectively. In some specific embodiments, provided herein is an antibody that binds to IL-23R, comprising: (i) a VH comprising an HCDR1, an HCDR2, and an HCDR3 having an amino acid sequence of SEQ ID NOs:348, 349, and 350, respectively, and (ii) an VL comprising an LCDR1, an LCDR2, and an LCDR3 having an amino acid sequence of SEQ ID NOs:351, 352, and 353, respectively. In some specific embodiments, provided herein is an antibody that binds to IL-23R, comprising: (i) a VH comprising an HCDR1, an HCDR2, and an HCDR3 having an amino acid sequence of SEQ ID NOs:354, 355, and 356, respectively, and (ii) an VL comprising an LCDR1, an LCDR2, and an LCDR3 having an amino acid sequence of SEQ ID NOs:357, 358, and 359, respectively. In some specific embodiments, provided herein is an antibody that binds to IL-23R, comprising: (i) a VH comprising an HCDR1, an HCDR2, and an HCDR3 having an amino acid sequence of SEQ ID NOs:360, 361, and 362, respectively, and (ii) an VL comprising an LCDR1, an LCDR2, and an LCDR3 having an amino acid sequence of SEQ ID NOs:363, 364, and 365, respectively. In some specific embodiments, provided herein is an antibody that binds to IL-23R, comprising: (i) a VH comprising an HCDR1, an HCDR2, and an HCDR3 having an amino acid sequence of SEQ ID NOs:366, 367, and 368, respectively, and (ii) an VL comprising an LCDR1, an LCDR2, and an LCDR3 having an amino acid sequence of SEQ ID NOs:369, 370, and 371, respectively. In some specific embodiments, provided herein is an antibody that binds to IL-23R, comprising: (i) a VH comprising an HCDR1, an HCDR2, and an HCDR3 having an amino acid sequence of SEQ ID NOs:372, 373, and 374, respectively, and (ii) an VL comprising an LCDR1, an LCDR2, and an LCDR3 having an amino acid sequence of SEQ ID NOs:375, 376, and 377, respectively. In some specific embodiments, provided herein is an antibody that binds to IL-23R, comprising: (i) a VH comprising an HCDR1, an HCDR2, and an HCDR3 having an amino acid sequence of SEQ ID NOs:378, 379, and 380, respectively, and (ii) an VL comprising an LCDR1, an LCDR2, and an LCDR3 having an amino acid sequence of SEQ ID NOs:381, 382, and 383, respectively. In some specific embodiments, provided herein is an antibody that binds to IL-23R, comprising: (i) a VH comprising an HCDR1, an HCDR2, and an HCDR3 having an amino acid sequence of SEQ ID NOs:384, 385, and 386, respectively, and (ii) an VL comprising an LCDR1, an LCDR2, and an LCDR3 having an amino acid sequence of SEQ ID NOs:387, 388, and 389, respectively. In some specific embodiments, provided herein is an antibody that binds to IL-23R, comprising: (i) a VH comprising an HCDR1, an HCDR2, and an HCDR3 having an amino acid sequence of SEQ ID NOs:390, 391, and 392, respectively, and (ii) an VL comprising an LCDR1, an LCDR2, and an LCDR3 having an amino acid sequence of SEQ ID NOs:393, 394, and 395, respectively. In some specific embodiments, provided herein is an antibody that binds to IL-23R, comprising: (i) a VH comprising an HCDR1, an HCDR2, and an HCDR3 having an amino acid sequence of SEQ ID NOs:396, 397, and 398, respectively, and (ii) an VL comprising an LCDR1, an LCDR2, and an LCDR3 having an amino acid sequence of SEQ ID NOs:399, 400, and 401, respectively. In some specific embodiments, provided herein is an antibody that binds to IL-23R, comprising: (i) a VH comprising an HCDR1, an HCDR2, and an HCDR3 having an amino acid sequence of SEQ ID NOs:402, 403, and 404, respectively, and (ii) an VL comprising an LCDR1, an LCDR2, and an LCDR3 having an amino acid sequence of SEQ ID NOs:405, 406, and 407, respectively. In some specific embodiments, provided herein is an antibody that binds to IL-23R, comprising: (i) a VH comprising an HCDR1, an HCDR2, and an HCDR3 having an amino acid sequence of SEQ ID NOs:408, 409, and 410, respectively, and (ii) an VL comprising an LCDR1, an LCDR2, and an LCDR3 having an amino acid sequence of SEQ ID NOs:411, 412, and 413, respectively. In some specific embodiments, provided herein is an antibody that binds to IL-23R, comprising: (i) a VH comprising an HCDR1, an HCDR2, and an HCDR3 having an amino acid sequence of SEQ ID NOs:420, 421, and 422, respectively, and (ii) an VL comprising an LCDR1, an LCDR2, and an LCDR3 having an amino acid sequence of SEQ ID NOs:423, 424, and 425, respectively. In some specific embodiments, provided herein is an antibody that binds to IL-23R, comprising: (i) a VH comprising an HCDR1, an HCDR2, and an HCDR3 having an amino acid sequence of SEQ ID NOs:426, 427, and 428, respectively, and (ii) an VL comprising an LCDR1, an LCDR2, and an LCDR3 having an amino acid sequence of SEQ ID NOs:429, 430, and 431, respectively. In some specific embodiments, provided herein is an antibody that binds to IL-23R, comprising: (i) a VH comprising an HCDR1, an HCDR2, and an HCDR3 having an amino acid sequence of SEQ ID NOs:432, 433, and 434, respectively, and (ii) an VL comprising an LCDR1, an LCDR2, and an LCDR3 having an amino acid sequence of SEQ ID NOs:435, 436, and 437, respectively. In some specific embodiments, provided herein is an antibody that binds to IL-23R, comprising: (i) a VH comprising an HCDR1, an HCDR2, and an HCDR3 having an amino acid sequence of SEQ ID NOs:438, 439, and 440, respectively, and (ii) an VL comprising an LCDR1, an LCDR2, and an LCDR3 having an amino acid sequence of SEQ ID NOs:441, 442, and 443, respectively. In some specific embodiments, provided herein is an antibody that binds to IL-23R, comprising: (i) a VH comprising an HCDR1, an HCDR2, and an HCDR3 having an amino acid sequence of SEQ ID NOs:444, 445, and 446, respectively, and (ii) an VL comprising an LCDR1, an LCDR2, and an LCDR3 having an amino acid sequence of SEQ ID NOs:447, 448, and 449, respectively. In some specific embodiments, provided herein is an antibody that binds to IL-23R, comprising: (i) a VH comprising an HCDR1, an HCDR2, and an HCDR3 having an amino acid sequence of SEQ ID NOs:450, 451, and 452, respectively, and (ii) an VL comprising an LCDR1, an LCDR2, and an LCDR3 having an amino acid sequence of SEQ ID NOs:453, 454, and 455, respectively. In some specific embodiments, provided herein is an antibody that binds to IL-23R, comprising: (i) a VH comprising an HCDR1, an HCDR2, and an HCDR3 having an amino acid sequence of SEQ ID NOs:456, 457, and 458, respectively, and (ii) an VL comprising an LCDR1, an LCDR2, and an LCDR3 having an amino acid sequence of SEQ ID NOs:459, 460, and 461, respectively. In some specific embodiments, provided herein is an antibody that binds to IL-23R, comprising: (i) a VH comprising an HCDR1, an HCDR2, and an HCDR3 having an amino acid sequence of SEQ ID NOs:462, 463, and 464, respectively, and (ii) an VL comprising an LCDR1, an LCDR2, and an LCDR3 having an amino acid sequence of SEQ ID NOs:465, 466, and 467, respectively. In some specific embodiments, provided herein is an antibody that binds to IL-23R, comprising: (i) a VH comprising an HCDR1, an HCDR2, and an HCDR3 having an amino acid sequence of SEQ ID NOs:468, 469, and 470, respectively, and (ii) an VL comprising an LCDR1, an LCDR2, and an LCDR3 having an amino acid sequence of SEQ ID NOs:471, 472, and 473, respectively. In some specific embodiments, provided herein is an antibody that binds to IL-23R, comprising: (i) a VH comprising an HCDR1, an HCDR2, and an HCDR3 having an amino acid sequence of SEQ ID NOs:474, 475, and 476, respectively, and (ii) an VL comprising an LCDR1, an LCDR2, and an LCDR3 having an amino acid sequence of SEQ ID NOs:477, 478, and 479, respectively. In some specific embodiments, provided herein is an antibody that binds to IL-23R, comprising: (i) a VH comprising an HCDR1, an HCDR2, and an HCDR3 having an amino acid sequence of SEQ ID NOs:480, 481, and 482, respectively, and (ii) an VL comprising an LCDR1, an LCDR2, and an LCDR3 having an amino acid sequence of SEQ ID NOs:483, 484, and 485, respectively. In some specific embodiments, provided herein is an antibody that binds to IL-23R, comprising: (i) a VH comprising an HCDR1, an HCDR2, and an HCDR3 having an amino acid sequence of SEQ ID NOs:486, 487, and 488, respectively, and (ii) an VL comprising an LCDR1, an LCDR2, and an LCDR3 having an amino acid sequence of SEQ ID NOs:489, 490, and 491, respectively. In some specific embodiments, provided herein is an antibody that binds to IL-23R, comprising: (i) a VH comprising an HCDR1, an HCDR2, and an HCDR3 having an amino acid sequence of SEQ ID NOs:492, 493, and 494, respectively, and (ii) an VL comprising an LCDR1, an LCDR2, and an LCDR3 having an amino acid sequence of SEQ ID NOs:495, 496, and 497, respectively. In some specific embodiments, provided herein is an antibody that binds to IL-23R, comprising: (i) a VH comprising an HCDR1, an HCDR2, and an HCDR3 having an amino acid sequence of SEQ ID NOs:498, 499, and 500, respectively, and (ii) an VL comprising an LCDR1, an LCDR2, and an LCDR3 having an amino acid sequence of SEQ ID NOs:501, 502, and 503, respectively. In some specific embodiments, provided herein is an antibody that binds to IL-23R, comprising: (i) a VH comprising an HCDR1, an HCDR2, and an HCDR3 having an amino acid sequence of SEQ ID NOs:504, 505, and 506, respectively, and (ii) an VL comprising an LCDR1, an LCDR2, and an LCDR3 having an amino acid sequence of SEQ ID NOs:507, 508, and 509, respectively. In some specific embodiments, provided herein is an antibody that binds to IL-23R, comprising: (i) a VH comprising an HCDR1, an HCDR2, and an HCDR3 having an amino acid sequence of SEQ ID NOs:510, 511, and 512, respectively, and (ii) an VL comprising an LCDR1, an LCDR2, and an LCDR3 having an amino acid sequence of SEQ ID NOs:513, 514, and 515, respectively. In some specific embodiments, provided herein is an antibody that binds to IL-23R, comprising: (i) a VH comprising an HCDR1, an HCDR2, and an HCDR3 having an amino acid sequence of SEQ ID NOs:516, 517, and 518, respectively, and (ii) an VL comprising an LCDR1, an LCDR2, and an LCDR3 having an amino acid sequence of SEQ ID NOs:519, 520, and 521, respectively. In some specific embodiments, provided herein is an antibody that binds to IL-23R, comprising: (i) a VH comprising an HCDR1, an HCDR2, and an HCDR3 having an amino acid sequence of SEQ ID NOs:522, 523, and 524, respectively, and (ii) an VL comprising an LCDR1, an LCDR2, and an LCDR3 having an amino acid sequence of SEQ ID NOs:525, 526, and 527, respectively. In some specific embodiments, provided herein is an antibody that binds to IL-23R, comprising: (i) a VH comprising an HCDR1, an HCDR2, and an HCDR3 having an amino acid sequence of SEQ ID NOs:528, 529, and 530, respectively, and (ii) an VL comprising an LCDR1, an LCDR2, and an LCDR3 having an amino acid sequence of SEQ ID NOs:531, 532, and 533, respectively. In some specific embodiments, provided herein is an antibody that binds to IL-23R, comprising: (i) a VH comprising an HCDR1, an HCDR2, and an HCDR3 having an amino acid sequence of SEQ ID NOs:540, 541, and 542, respectively, and (ii) an VL comprising an LCDR1, an LCDR2, and an LCDR3 having an amino acid sequence of SEQ ID NOs:543, 544, and 545, respectively. In some specific embodiments, provided herein is an antibody that binds to IL-23R, comprising: (i) a VH comprising an HCDR1, an HCDR2, and an HCDR3 having an amino acid sequence of SEQ ID NOs:546, 547, and 548, respectively, and (ii) an VL comprising an LCDR1, an LCDR2, and an LCDR3 having an amino acid sequence of SEQ ID NOs:549, 550, and 551, respectively. In some specific embodiments, provided herein is an antibody that binds to IL-23R, comprising: (i) a VH comprising an HCDR1, an HCDR2, and an HCDR3 having an amino acid sequence of SEQ ID NOs:552, 553, and 554, respectively, and (ii) an VL comprising an LCDR1, an LCDR2, and an LCDR3 having an amino acid sequence of SEQ ID NOs:555, 556, and 557, respectively. In some specific embodiments, provided herein is an antibody that binds to IL-23R, comprising: (i) a VH comprising an HCDR1, an HCDR2, and an HCDR3 having an amino acid sequence of SEQ ID NOs:558, 559, and 560, respectively, and (ii) an VL comprising an LCDR1, an LCDR2, and an LCDR3 having an amino acid sequence of SEQ ID NOs:561, 562, and 563, respectively. In some specific embodiments, provided herein is an antibody that binds to IL-23R, comprising: (i) a VH comprising an HCDR1, an HCDR2, and an HCDR3 having an amino acid sequence of SEQ ID NOs:564, 565, and 566, respectively, and (ii) an VL comprising an LCDR1, an LCDR2, and an LCDR3 having an amino acid sequence of SEQ ID NOs:567, 568, and 569, respectively. In some specific embodiments, provided herein is an antibody that binds to IL-23R, comprising: (i) a VH comprising an HCDR1, an HCDR2, and an HCDR3 having an amino acid sequence of SEQ ID NOs:570, 571, and 572, respectively, and (ii) an VL comprising an LCDR1, an LCDR2, and an LCDR3 having an amino acid sequence of SEQ ID NOs:573, 574, and 575, respectively. In some specific embodiments, provided herein is an antibody that binds to IL-23R, comprising: (i) a VH comprising an HCDR1, an HCDR2, and an HCDR3 having an amino acid sequence of SEQ ID NOs:576, 577, and 578, respectively, and (ii) an VL comprising an LCDR1, an LCDR2, and an LCDR3 having an amino acid sequence of SEQ ID NOs:579, 580, and 581, respectively. In some specific embodiments, provided herein is an antibody that binds to IL-23R, comprising: (i) a VH comprising an HCDR1, an HCDR2, and an HCDR3 having an amino acid sequence of SEQ ID NOs:582, 583, and 584, respectively, and (ii) an VL comprising an LCDR1, an LCDR2, and an LCDR3 having an amino acid sequence of SEQ ID NOs:585, 586, and 587, respectively. In some specific embodiments, provided herein is an antibody that binds to IL-23R, comprising: (i) a VH comprising an HCDR1, an HCDR2, and an HCDR3 having an amino acid sequence of SEQ ID NOs:600, 601, and 602, respectively, and (ii) an VL comprising an LCDR1, an LCDR2, and an LCDR3 having an amino acid sequence of SEQ ID NOs:603, 604, and 605, respectively. In some specific embodiments, provided herein is an antibody that binds to IL-23R, comprising: (i) a VH comprising an HCDR1, an HCDR2, and an HCDR3 having an amino acid sequence of SEQ ID NOs:606, 607, and 608, respectively, and (ii) an VL comprising an LCDR1, an LCDR2, and an LCDR3 having an amino acid sequence of SEQ ID NOs:609, 610, and 611, respectively. In some specific embodiments, provided herein is an antibody that binds to IL-23R, comprising: (i) a VH comprising an HCDR1, an HCDR2, and an HCDR3 having an amino acid sequence of SEQ ID NOs:612, 613, and 614, respectively, and (ii) an VL comprising an LCDR1, an LCDR2, and an LCDR3 having an amino acid sequence of SEQ ID NOs:615, 616, and 617, respectively. In some specific embodiments, provided herein is an antibody that binds to IL-23R, comprising: (i) a VH comprising an HCDR1, an HCDR2, and an HCDR3 having an amino acid sequence of SEQ ID NOs:618, 619, and 620, respectively, and (ii) an VL comprising an LCDR1, an LCDR2, and an LCDR3 having an amino acid sequence of SEQ ID NOs:621, 622, and 623, respectively. In some specific embodiments, provided herein is an antibody that binds to IL-23R, comprising: (i) a VH comprising an HCDR1, an HCDR2, and an HCDR3 having an amino acid sequence of SEQ ID NOs:624, 625, and 626, respectively, and (ii) an VL comprising an LCDR1, an LCDR2, and an LCDR3 having an amino acid sequence of SEQ ID NOs:627, 628, and 629, respectively. In some specific embodiments, provided herein is an antibody that binds to IL-23R, comprising: (i) a VH comprising an HCDR1, an HCDR2, and an HCDR3 having an amino acid sequence of SEQ ID NOs:630, 631, and 632, respectively, and (ii) an VL comprising an LCDR1, an LCDR2, and an LCDR3 having an amino acid sequence of SEQ ID NOs:633, 634, and 635, respectively. In some specific embodiments, provided herein is an antibody that binds to IL-23R, comprising: (i) a VH comprising an HCDR1, an HCDR2, and an HCDR3 having an amino acid sequence of SEQ ID NOs:636, 637, and 638, respectively, and (ii) an VL comprising an LCDR1, an LCDR2, and an LCDR3 having an amino acid sequence of SEQ ID NOs:639, 640, and 641, respectively. In some specific embodiments, provided herein is an antibody that binds to IL-23R, comprising: (i) a VH comprising an HCDR1, an HCDR2, and an HCDR3 having an amino acid sequence of SEQ ID NOs:642, 643, and 644, respectively, and (ii) an VL comprising an LCDR1, an LCDR2, and an LCDR3 having an amino acid sequence of SEQ ID NOs:645, 646, and 647, respectively. In some specific embodiments, provided herein is an antibody that binds to IL-23R, comprising: (i) a VH comprising an HCDR1, an HCDR2, and an HCDR3 having an amino acid sequence of SEQ ID NOs:648, 649, and 650, respectively, and (ii) an VL comprising an LCDR1, an LCDR2, and an LCDR3 having an amino acid sequence of SEQ ID NOs:651, 652, and 653, respectively. In some specific embodiments, provided herein is an antibody that binds to IL-23R, comprising: (i) a VH comprising an HCDR1, an HCDR2, and an HCDR3 having an amino acid sequence of SEQ ID NOs:654, 655, and 656, respectively, and (ii) an VL comprising an LCDR1, an LCDR2, and an LCDR3 having an amino acid sequence of SEQ ID NOs:657, 658, and 659, respectively. In some specific embodiments, provided herein is an antibody that binds to IL-23R, comprising: (i) a VH comprising an HCDR1, an HCDR2, and an HCDR3 having an amino acid sequence of SEQ ID NOs:666, 667, and 668, respectively, and (ii) an VL comprising an LCDR1, an LCDR2, and an LCDR3 having an amino acid sequence of SEQ ID NOs:669, 670, and 671, respectively. In some specific embodiments, provided herein is an antibody that binds to IL-23R, comprising: (i) a VH comprising an HCDR1, an HCDR2, and an HCDR3 having an amino acid sequence of SEQ ID NOs:900, 901, and 902, respectively, and (ii) an VL comprising an LCDR1, an LCDR2, and an LCDR3 having an amino acid sequence of SEQ ID NOs:903, 904, and 905, respectively. In some specific embodiments, provided herein is an antibody that binds to IL-23R, comprising: (i) a VH comprising an HCDR1, an HCDR2, and an HCDR3 having an amino acid sequence of SEQ ID NOs:906, 907, and 908, respectively, and (ii) an VL comprising an LCDR1, an LCDR2, and an LCDR3 having an amino acid sequence of SEQ ID NOs:909, 910, and 911, respectively. In some specific embodiments, provided herein is an antibody that binds to IL-23R, comprising: (i) a VH comprising an HCDR1, an HCDR2, and an HCDR3 having an amino acid sequence of SEQ ID NOs:912, 913, and 914, respectively, and (ii) an VL comprising an LCDR1, an LCDR2, and an LCDR3 having an amino acid sequence of SEQ ID NOs:915, 916, and 917, respectively. In some specific embodiments, provided herein is an antibody that binds to IL-23R, comprising: (i) a VH comprising an HCDR1, an HCDR2, and an HCDR3 having an amino acid sequence of SEQ ID NOs:918, 919, and 920, respectively, and (ii) an VL comprising an LCDR1, an LCDR2, and an LCDR3 having an amino acid sequence of SEQ ID NOs:921, 922, and 923, respectively. In some specific embodiments, provided herein is an antibody that binds to IL-23R, comprising: (i) a VH comprising an HCDR1, an HCDR2, and an HCDR3 having an amino acid sequence of SEQ ID NOs:936, 937, and 938, respectively, and (ii) an VL comprising an LCDR1, an LCDR2, and an LCDR3 having an amino acid sequence of SEQ ID NOs:939, 940, and 941, respectively. In some specific embodiments, provided herein is an antibody that binds to IL-23R, comprising: (i) a VH comprising an HCDR1, an HCDR2, and an HCDR3 having an amino acid sequence of SEQ ID NOs:960, 961, and 962, respectively, and (ii) an VL comprising an LCDR1, an LCDR2, and an LCDR3 having an amino acid sequence of SEQ ID NOs:963, 964, and 965, respectively. In some specific embodiments, provided herein is an antibody that binds to IL-23R, comprising: (i) a VH comprising an HCDR1, an HCDR2, and an HCDR3 having an amino acid sequence of SEQ ID NOs:966, 967, and 968, respectively, and (ii) an VL comprising an LCDR1, an LCDR2, and an LCDR3 having an amino acid sequence of SEQ ID NOs:969, 970, and 971, respectively. In some specific embodiments, provided herein is an antibody that binds to IL-23R, comprising: (i) a VH comprising an HCDR1, an HCDR2, and an HCDR3 having an amino acid sequence of SEQ ID NOs:972, 973, and 974, respectively, and (ii) an VL comprising an LCDR1, an LCDR2, and an LCDR3 having an amino acid sequence of SEQ ID NOs:975, 976, and 977, respectively. In some specific embodiments, provided herein is an antibody that binds to IL-23R, comprising: (i) a VH comprising an HCDR1, an HCDR2, and an HCDR3 having an amino acid sequence of SEQ ID NOs:978, 979, and 980, respectively, and (ii) an VL comprising an LCDR1, an LCDR2, and an LCDR3 having an amino acid sequence of SEQ ID NOs:981, 982, and 983, respectively. In some specific embodiments, provided herein is an antibody that binds to IL-23R, comprising: (i) a VH comprising an HCDR1, an HCDR2, and an HCDR3 having an amino acid sequence of SEQ ID NOs:984, 985, and 986, respectively, and (ii) an VL comprising an LCDR1, an LCDR2, and an LCDR3 having an amino acid sequence of SEQ ID NOs:987, 988, and 989, respectively. In some specific embodiments, provided herein is an antibody that binds to IL-23R, comprising: (i) a VH comprising an HCDR1, an HCDR2, and an HCDR3 having an amino acid sequence of SEQ ID NOs:990, 991, and 992, respectively, and (ii) an VL comprising an LCDR1, an LCDR2, and an LCDR3 having an amino acid sequence of SEQ ID NOs:993, 994, and 995, respectively. In some specific embodiments, provided herein is an antibody that binds to IL-23R, comprising: (i) a VH comprising an HCDR1, an HCDR2, and an HCDR3 having an amino acid sequence of SEQ ID NOs:996, 997, and 998, respectively, and (ii) an VL comprising an LCDR1, an LCDR2, and an LCDR3 having an amino acid sequence of SEQ ID NOs:999, 1000, and 1001, respectively. In some specific embodiments, provided herein is an antibody that binds to IL-23R, comprising: (i) a VH comprising an HCDR1, an HCDR2, and an HCDR3 having an amino acid sequence of SEQ ID NOs:1002, 1003, and 1004, respectively, and (ii) an VL comprising an LCDR1, an LCDR2, and an LCDR3 having an amino acid sequence of SEQ ID NOs:1005, 1006, and 1007, respectively. In some specific embodiments, provided herein is an antibody that binds to IL-23R, comprising: (i) a VH comprising an HCDR1, an HCDR2, and an HCDR3 having an amino acid sequence of SEQ ID NOs:1008, 1009, and 1010, respectively, and (ii) an VL comprising an LCDR1, an LCDR2, and an LCDR3 having an amino acid sequence of SEQ ID NOs:1011, 1012, and 1013, respectively. In some specific embodiments, provided herein is an antibody that binds to IL-23R, comprising: (i) a VH comprising an HCDR1, an HCDR2, and an HCDR3 having an amino acid sequence of SEQ ID NOs:1014, 1015, and 1016, respectively, and (ii) an VL comprising an LCDR1, an LCDR2, and an LCDR3 having an amino acid sequence of SEQ ID NOs:1017, 1018, and 1019, respectively. In some specific embodiments, provided herein is an antibody that binds to IL-23R, comprising: (i) a VH comprising an HCDR1, an HCDR2, and an HCDR3 having an amino acid sequence of SEQ ID NOs:1020, 1021, and 1022, respectively, and (ii) an VL comprising an LCDR1, an LCDR2, and an LCDR3 having an amino acid sequence of SEQ ID NOs:1023, 1024, and 1025, respectively. In some specific embodiments, provided herein is an antibody that binds to IL-23R, comprising: (i) a VH comprising an HCDR1, an HCDR2, and an HCDR3 having an amino acid sequence of SEQ ID NOs:1026, 1027, and 1028, respectively, and (ii) an VL comprising an LCDR1, an LCDR2, and an LCDR3 having an amino acid sequence of SEQ ID NOs:1029, 1030, and 1031, respectively. In some specific embodiments, provided herein is an antibody that binds to IL-23R, comprising: (i) a VH comprising an HCDR1, an HCDR2, and an HCDR3 having an amino acid sequence of SEQ ID NOs:1032, 1033, and 1034, respectively, and (ii) an VL comprising an LCDR1, an LCDR2, and an LCDR3 having an amino acid sequence of SEQ ID NOs:1035, 1036, and 1037, respectively. In some specific embodiments, provided herein is an antibody that binds to IL-23R, comprising: (i) a VH comprising an HCDR1, an HCDR2, and an HCDR3 having an amino acid sequence of SEQ ID NOs:1038, 1039, and 1040, respectively, and (ii) an VL comprising an LCDR1, an LCDR2, and an LCDR3 having an amino acid sequence of SEQ ID NOs:1041, 1042, and 1043, respectively. In some specific embodiments, provided herein is an antibody that binds to IL-23R, comprising: (i) a VH comprising an HCDR1, an HCDR2, and an HCDR3 having an amino acid sequence of SEQ ID NOs:1050, 1051, and 1052, respectively, and (ii) an VL comprising an LCDR1, an LCDR2, and an LCDR3 having an amino acid sequence of SEQ ID NOs:1053, 1054, and 1055, respectively. In some specific embodiments, provided herein is an antibody that binds to IL-23R, comprising: (i) a VH comprising an HCDR1, an HCDR2, and an HCDR3 having an amino acid sequence of SEQ ID NOs:1056, 1057, and 1058, respectively, and (ii) an VL comprising an LCDR1, an LCDR2, and an LCDR3 having an amino acid sequence of SEQ ID NOs:1059, 1060, and 1061, respectively. In some specific embodiments, provided herein is an antibody that binds to IL-23R, comprising: (i) a VH comprising an HCDR1, an HCDR2, and an HCDR3 having an amino acid sequence of SEQ ID NOs:1062, 1063, and 1064, respectively, and (ii) an VL comprising an LCDR1, an LCDR2, and an LCDR3 having an amino acid sequence of SEQ ID NOs:1065, 1066, and 1067, respectively. In some specific embodiments, provided herein is an antibody that binds to IL-23R, comprising: (i) a VH comprising an HCDR1, an HCDR2, and an HCDR3 having an amino acid sequence of SEQ ID NOs:1068, 1069, and 1070, respectively, and (ii) an VL comprising an LCDR1, an LCDR2, and an LCDR3 having an amino acid sequence of SEQ ID NOs:1071, 1072, and 1073, respectively. In some specific embodiments, provided herein is an antibody that binds to IL-23R, comprising: (i) a VH comprising an HCDR1, an HCDR2, and an HCDR3 having an amino acid sequence of SEQ ID NOs:1080, 1081, and 1082, respectively, and (ii) a VL comprising an LCDR1, an LCDR2, and an LCDR3 having an amino acid sequence of SEQ ID NOs:1083, 1084, and 1085, respectively. In some specific embodiments, provided herein is an antibody that binds to IL-23R, comprising: (i) a VH comprising an HCDR1, an HCDR2, and an HCDR3 having an amino acid sequence of SEQ ID NOs:1086, 1087, and 1088, respectively, and (ii) an VL comprising an LCDR1, an LCDR2, and an LCDR3 having an amino acid sequence of SEQ ID NOs:1089, 1090, and 1091, respectively. In some specific embodiments, provided herein is an antibody that binds to IL-23R, comprising: (i) a VH comprising an HCDR1, an HCDR2, and an HCDR3 having an amino acid sequence of SEQ ID NOs:1092, 1093, and 1094, respectively, and (ii) an VL comprising an LCDR1, an LCDR2, and an LCDR3 having an amino acid sequence of SEQ ID NOs:1095, 1096, and 1097, respectively. In some specific embodiments, provided herein is an antibody that binds to IL-23R, comprising: (i) a VH comprising an HCDR1, an HCDR2, and an HCDR3 having an amino acid sequence of SEQ ID NOs:1098, 1099, and 1100, respectively, and (ii) an VL comprising an LCDR1, an LCDR2, and an LCDR3 having an amino acid sequence of SEQ ID NOs:1101, 1102, and 1103, respectively. In some specific embodiments, provided herein is an antibody that binds to IL-23R, comprising: (i) a VH comprising an HCDR1, an HCDR2, and an HCDR3 having an amino acid sequence of SEQ ID NOs:1104, 1105, and 1106, respectively, and (ii) an VL comprising an LCDR1, an LCDR2, and an LCDR3 having an amino acid sequence of SEQ ID NOs:1107, 1108, and 1109, respectively. In some specific embodiments, provided herein is an antibody that binds to IL-23R, comprising: (i) a VH comprising an HCDR1, an HCDR2, and an HCDR3 having an amino acid sequence of SEQ ID NOs:1110, 1111, and 1112, respectively, and (ii) an VL comprising an LCDR1, an LCDR2, and an LCDR3 having an amino acid sequence of SEQ ID NOs:1113, 1114, and 1115, respectively. In some specific embodiments, provided herein is an antibody that binds to IL-23R, comprising: (i) a VH comprising an HCDR1, an HCDR2, and an HCDR3 having an amino acid sequence of SEQ ID NOs:1116, 1117, and 1118, respectively, and (ii) an VL comprising an LCDR1, an LCDR2, and an LCDR3 having an amino acid sequence of SEQ ID NOs:1119, 1120, and 1121, respectively. In some specific embodiments, provided herein is an antibody that binds to IL-23R, comprising: (i) a VH comprising an HCDR1, an HCDR2, and an HCDR3 having an amino acid sequence of SEQ ID NOs:1122, 1123, and 1124, respectively, and (ii) an VL comprising an LCDR1, an LCDR2, and an LCDR3 having an amino acid sequence of SEQ ID NOs:1125, 1126, and 1127, respectively. In some specific embodiments, provided herein is an antibody that binds to IL-23R, comprising: (i) a VH comprising an HCDR1, an HCDR2, and an HCDR3 having an amino acid sequence of SEQ ID NOs:1128, 1129, and 1130, respectively, and (ii) an VL comprising an LCDR1, an LCDR2, and an LCDR3 having an amino acid sequence of SEQ ID NOs:1131, 1132, and 1133, respectively. In some specific embodiments, provided herein is an antibody that binds to IL-23R, comprising: (i) a VH comprising an HCDR1, an HCDR2, and an HCDR3 having an amino acid sequence of SEQ ID NOs:1134, 1135, and 1136, respectively, and (ii) a VL comprising an LCDR1, an LCDR2, and an LCDR3 having an amino acid sequence of SEQ ID NOs:1137, 1138, and 1139, respectively. In some specific embodiments, provided herein is an antibody that binds to IL-23R, comprising: (i) a VH comprising an HCDR1, an HCDR2, and an HCDR3 having an amino acid sequence of SEQ ID NOs:1140, 1141, and 1142, respectively, and (ii) a VL comprising an LCDR1, an LCDR2, and an LCDR3 having an amino acid sequence of SEQ ID NOs:1143, 1144, and 1145, respectively. In some specific embodiments, provided herein is an antibody that binds to IL-23R, comprising: (i) a VH comprising an HCDR1, an HCDR2, and an HCDR3 having an amino acid sequence of SEQ ID NOs:1146, 1147, and 1148, respectively, and (ii) an VL comprising an LCDR1, an LCDR2, and an LCDR3 having an amino acid sequence of SEQ ID NOs:1149, 1150, and 1151, respectively. In some specific embodiments, provided herein is an antibody that binds to IL-23R, comprising: (i) a VH comprising an HCDR1, an HCDR2, and an HCDR3 having an amino acid sequence of SEQ ID NOs:1152, 1153, and 1154, respectively, and (ii) an VL comprising an LCDR1, an LCDR2, and an LCDR3 having an amino acid sequence of SEQ ID NOs:1155, 1156, and 1157, respectively. In some specific embodiments, provided herein is an antibody that binds to IL-23R, comprising: (i) a VH comprising an HCDR1, an HCDR2, and an HCDR3 having an amino acid sequence of SEQ ID NOs:1158, 1159, and 1160, respectively, and (ii) an VL comprising an LCDR1, an LCDR2, and an LCDR3 having an amino acid sequence of SEQ ID NOs:1161, 1162, and 1163, respectively. In some specific embodiments, provided herein is an antibody that binds to IL-23R, comprising: (i) a VH comprising an HCDR1, an HCDR2, and an HCDR3 having an amino acid sequence of SEQ ID NOs:1164, 1165, and 1166, respectively, and (ii) an VL comprising an LCDR1, an LCDR2, and an LCDR3 having an amino acid sequence of SEQ ID NOs:1167, 1168, and 1169, respectively. In some specific embodiments, provided herein is an antibody that binds to IL-23R, comprising: (i) a VH comprising an HCDR1, an HCDR2, and an HCDR3 having an amino acid sequence of SEQ ID NOs:1170, 1171, and 1172, respectively, and (ii) an VL comprising an LCDR1, an LCDR2, and an LCDR3 having an amino acid sequence of SEQ ID NOs:1173, 1174, and 1175, respectively. In some specific embodiments, provided herein is an antibody that binds to IL-23R, comprising: (i) a VH comprising an HCDR1, an HCDR2, and an HCDR3 having an amino acid sequence of SEQ ID NOs:1176, 1177, and 1178, respectively, and (ii) an VL comprising an LCDR1, an LCDR2, and an LCDR3 having an amino acid sequence of SEQ ID NOs:1179, 1180, and 1181, respectively. In some specific embodiments, provided herein is an antibody that binds to IL-23R, comprising: (i) a VH comprising an HCDR1, an HCDR2, and an HCDR3 having an amino acid sequence of SEQ ID NOs:1182, 1183, and 1184, respectively, and (ii) an VL comprising an LCDR1, an LCDR2, and an LCDR3 having an amino acid sequence of SEQ ID NOs:1185, 1186, and 1187, respectively. In some specific embodiments, provided herein is an antibody that binds to IL-23R, comprising: (i) a VH comprising an HCDR1, an HCDR2, and an HCDR3 having an amino acid sequence of SEQ ID NOs:1188, 1189, and 1190, respectively, and (ii) an VL comprising an LCDR1, an LCDR2, and an LCDR3 having an amino acid sequence of SEQ ID NOs:1191, 1192, and 1193, respectively. In some specific embodiments, provided herein is an antibody that binds to IL-23R, comprising: (i) a VH comprising an HCDR1, an HCDR2, and an HCDR3 having an amino acid sequence of SEQ ID NOs:1194, 1195, and 1196, respectively, and (ii) an VL comprising an LCDR1, an LCDR2, and an LCDR3 having an amino acid sequence of SEQ ID NOs:1197, 1198, and 1199, respectively. In some specific embodiments, provided herein is an antibody that binds to IL-23R, comprising: (i) a VH comprising an HCDR1, an HCDR2, and an HCDR3 having an amino acid sequence of SEQ ID NOs:1200, 1201, and 1202, respectively, and (ii) an VL comprising an LCDR1, an LCDR2, and an LCDR3 having an amino acid sequence of SEQ ID NOs:1203, 1204, and 1205, respectively. In some specific embodiments, provided herein is an antibody that binds to IL-23R, comprising: (i) a VH comprising an HCDR1, an HCDR2, and an HCDR3 having an amino acid sequence of SEQ ID NOs:1206, 1207, and 1208, respectively, and (ii) an VL comprising an LCDR1, an LCDR2, and an LCDR3 having an amino acid sequence of SEQ ID NOs:1209, 1210, and 1211, respectively. In some specific embodiments, provided herein is an antibody that binds to IL-23R, comprising: (i) a VH comprising an HCDR1, an HCDR2, and an HCDR3 having an amino acid sequence of SEQ ID NOs:1212, 1213, and 1214, respectively, and (ii) an VL comprising an LCDR1, an LCDR2, and an LCDR3 having an amino acid sequence of SEQ ID NOs:1215, 1216, and 1217, respectively.

In some embodiments, the antibody further comprises one or more framework regions of SEQ ID NOs:136-269, 786-899. In some embodiments, the antibody provided herein is a humanized antibody. In some embodiments, the antibody is a chimeric antibody. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, the antibody or antigen binding fragment provided herein comprises a VH comprising the amino acid sequence of SEQ ID NO:138, and a VL comprising the amino acid sequence of SEQ ID NO:139. In some embodiments, the antibody or antigen binding fragment provided herein comprises a VH comprising the amino acid sequence of SEQ ID NO:142, and a VL comprising the amino acid sequence of SEQ ID NO:143. In some embodiments, the antibody or antigen binding fragment provided herein comprises a VH comprising the amino acid sequence of SEQ ID NO:144, and a VL comprising the amino acid sequence of SEQ ID NO: 145. In some embodiments, the antibody or antigen binding fragment provided herein comprises a VH comprising the amino acid sequence of SEQ ID NO:148, and a VL comprising the amino acid sequence of SEQ ID NO: 149. In some embodiments, the antibody or antigen binding fragment provided herein comprises a VH comprising the amino acid sequence of SEQ ID NO:150, and a VL comprising the amino acid sequence of SEQ ID NO:151. In some embodiments, the antibody or antigen binding fragment provided herein comprises a VH comprising the amino acid sequence of SEQ ID NO:152, and a VL comprising the amino acid sequence of SEQ ID NO:153. In some embodiments, the antibody or antigen binding fragment provided herein comprises a VH comprising the amino acid sequence of SEQ ID NO:154, and a VL comprising the amino acid sequence of SEQ ID NO:155. In some embodiments, the antibody or antigen binding fragment provided herein comprises a VH comprising the amino acid sequence of SEQ ID NO:156, and a VL comprising the amino acid sequence of SEQ ID NO:157. In some embodiments, the antibody or antigen binding fragment provided herein comprises a VH comprising the amino acid sequence of SEQ ID NO:158, and a VL comprising the amino acid sequence of SEQ ID NO:159. In some embodiments, the antibody or antigen binding fragment provided herein comprises a VH comprising the amino acid sequence of SEQ ID NO:160, and a VL comprising the amino acid sequence of SEQ ID NO:161. In some embodiments, the antibody or antigen binding fragment provided herein comprises a VH comprising the amino acid sequence of SEQ ID NO:162, and a VL comprising the amino acid sequence of SEQ ID NO: 163. In some embodiments, the antibody or antigen binding fragment provided herein comprises a VH comprising the amino acid sequence of SEQ ID NO:164, and a VL comprising the amino acid sequence of SEQ ID NO: 165. In some embodiments, the antibody or antigen binding fragment provided herein comprises a VH comprising the amino acid sequence of SEQ ID NO:166, and a VL comprising the amino acid sequence of SEQ ID NO: 167. In some embodiments, the antibody or antigen binding fragment provided herein comprises a VH comprising the amino acid sequence of SEQ ID NO:168, and a VL comprising the amino acid sequence of SEQ ID NO:169. In some embodiments, the antibody or antigen binding fragment provided herein comprises a VH comprising the amino acid sequence of SEQ ID NO:170, and a VL comprising the amino acid sequence of SEQ ID NO:171. In some embodiments, the antibody or antigen binding fragment provided herein comprises a VH comprising the amino acid sequence of SEQ ID NO:172, and a VL comprising the amino acid sequence of SEQ ID NO:173. In some embodiments, the antibody or antigen binding fragment provided herein comprises a VH comprising the amino acid sequence of SEQ ID NO:174, and a VL comprising the amino acid sequence of SEQ ID NO:175. In some embodiments, the antibody or antigen binding fragment provided herein comprises a VH comprising the amino acid sequence of SEQ ID NO:176, and a VL comprising the amino acid sequence of SEQ ID NO: 177. In some embodiments, the antibody or antigen binding fragment provided herein comprises a VH comprising the amino acid sequence of SEQ ID NO:178, and a VL comprising the amino acid sequence of SEQ ID NO:179. In some embodiments, the antibody or antigen binding fragment provided herein comprises a VH comprising the amino acid sequence of SEQ ID NO:180, and a VL comprising the amino acid sequence of SEQ ID NO:181. In some embodiments, the antibody or antigen binding fragment provided herein comprises a VH comprising the amino acid sequence of SEQ ID NO:182, and a VL comprising the amino acid sequence of SEQ ID NO:183. In some embodiments, the antibody or antigen binding fragment provided herein comprises a VH comprising the amino acid sequence of SEQ ID NO:186, and a VL comprising the amino acid sequence of SEQ ID NO:187. In some embodiments, the antibody or antigen binding fragment provided herein comprises a VH comprising the amino acid sequence of SEQ ID NO:188, and a VL comprising the amino acid sequence of SEQ ID NO:189. In some embodiments, the antibody or antigen binding fragment provided herein comprises a VH comprising the amino acid sequence of SEQ ID NO:190, and a VL comprising the amino acid sequence of SEQ ID NO:191. In some embodiments, the antibody or antigen binding fragment provided herein comprises a VH comprising the amino acid sequence of SEQ ID NO:192, and a VL comprising the amino acid sequence of SEQ ID NO:193. In some embodiments, the antibody or antigen binding fragment provided herein comprises a VH comprising the amino acid sequence of SEQ ID NO:194, and a VL comprising the amino acid sequence of SEQ ID NO: 195. In some embodiments, the antibody or antigen binding fragment provided herein comprises a VH comprising the amino acid sequence of SEQ ID NO:196, and a VL comprising the amino acid sequence of SEQ ID NO: 197. In some embodiments, the antibody or antigen binding fragment provided herein comprises a VH comprising the amino acid sequence of SEQ ID NO:198, and a VL comprising the amino acid sequence of SEQ ID NO:199. In some embodiments, the antibody or antigen binding fragment provided herein comprises a VH comprising the amino acid sequence of SEQ ID NO:200, and a VL comprising the amino acid sequence of SEQ ID NO:201. In some embodiments, the antibody or antigen binding fragment provided herein comprises a VH comprising the amino acid sequence of SEQ ID NO:202, and a VL comprising the amino acid sequence of SEQ ID NO:203. In some embodiments, the antibody or antigen binding fragment provided herein comprises a VH comprising the amino acid sequence of SEQ ID NO:204, and a VL comprising the amino acid sequence of SEQ ID NO:205. In some embodiments, the antibody or antigen binding fragment provided herein comprises a VH comprising the amino acid sequence of SEQ ID NO:206, and a VL comprising the amino acid sequence of SEQ ID NO:207. In some embodiments, the antibody or antigen binding fragment provided herein comprises a VH comprising the amino acid sequence of SEQ ID NO:208, and a VL comprising the amino acid sequence of SEQ ID NO:209. In some embodiments, the antibody or antigen binding fragment provided herein comprises a VH comprising the amino acid sequence of SEQ ID NO:210, and a VL comprising the amino acid sequence of SEQ ID NO:211. In some embodiments, the antibody or antigen binding fragment provided herein comprises a VH comprising the amino acid sequence of SEQ ID NO:212, and a VL comprising the amino acid sequence of SEQ ID NO:213. In some embodiments, the antibody or antigen binding fragment provided herein comprises a VH comprising the amino acid sequence of SEQ ID NO:214, and a VL comprising the amino acid sequence of SEQ ID NO:215. In some embodiments, the antibody or antigen binding fragment provided herein comprises a VH comprising the amino acid sequence of SEQ ID NO:216, and a VL comprising the amino acid sequence of SEQ ID NO:217. In some embodiments, the antibody or antigen binding fragment provided herein comprises a VH comprising the amino acid sequence of SEQ ID NO:218, and a VL comprising the amino acid sequence of SEQ ID NO:219. In some embodiments, the antibody or antigen binding fragment provided herein comprises a VH comprising the amino acid sequence of SEQ ID NO:220, and a VL comprising the amino acid sequence of SEQ ID NO:221. In some embodiments, the antibody or antigen binding fragment provided herein comprises a VH comprising the amino acid sequence of SEQ ID NO:222, and a VL comprising the amino acid sequence of SEQ ID NO:223. In some embodiments, the antibody or antigen binding fragment provided herein comprises a VH comprising the amino acid sequence of SEQ ID NO:226, and a VL comprising the amino acid sequence of SEQ ID NO:227. In some embodiments, the antibody or antigen binding fragment provided herein comprises a VH comprising the amino acid sequence of SEQ ID NO:228, and a VL comprising the amino acid sequence of SEQ ID NO:229. In some embodiments, the antibody or antigen binding fragment provided herein comprises a VH comprising the amino acid sequence of SEQ ID NO:230, and a VL comprising the amino acid sequence of SEQ ID NO:231. In some embodiments, the antibody or antigen binding fragment provided herein comprises a VH comprising the amino acid sequence of SEQ ID NO:232, and a VL comprising the amino acid sequence of SEQ ID NO:233. In some embodiments, the antibody or antigen binding fragment provided herein comprises a VH comprising the amino acid sequence of SEQ ID NO:234, and a VL comprising the amino acid sequence of SEQ ID NO:235. In some embodiments, the antibody or antigen binding fragment provided herein comprises a VH comprising the amino acid sequence of SEQ ID NO:236, and a VL comprising the amino acid sequence of SEQ ID NO:237. In some embodiments, the antibody or antigen binding fragment provided herein comprises a VH comprising the amino acid sequence of SEQ ID NO:238, and a VL comprising the amino acid sequence of SEQ ID NO:239. In some embodiments, the antibody or antigen binding fragment provided herein comprises a VH comprising the amino acid sequence of SEQ ID NO:240, and a VL comprising the amino acid sequence of SEQ ID NO:241. In some embodiments, the antibody or antigen binding fragment provided herein comprises a VH comprising the amino acid sequence of SEQ ID NO:246, and a VL comprising the amino acid sequence of SEQ ID NO:247. In some embodiments, the antibody or antigen binding fragment provided herein comprises a VH comprising the amino acid sequence of SEQ ID NO:248, and a VL comprising the amino acid sequence of SEQ ID NO:249. In some embodiments, the antibody or antigen binding fragment provided herein comprises a VH comprising the amino acid sequence of SEQ ID NO:250, and a VL comprising the amino acid sequence of SEQ ID NO:251. In some embodiments, the antibody or antigen binding fragment provided herein comprises a VH comprising the amino acid sequence of SEQ ID NO:252, and a VL comprising the amino acid sequence of SEQ ID NO:253. In some embodiments, the antibody or antigen binding fragment provided herein comprises a VH comprising the amino acid sequence of SEQ ID NO:254, and a VL comprising the amino acid sequence of SEQ ID NO:255. In some embodiments, the antibody or antigen binding fragment provided herein comprises a VH comprising the amino acid sequence of SEQ ID NO:256, and a VL comprising the amino acid sequence of SEQ ID NO:257. In some embodiments, the antibody or antigen binding fragment provided herein comprises a VH comprising the amino acid sequence of SEQ ID NO:258, and a VL comprising the amino acid sequence of SEQ ID NO:259. In some embodiments, the antibody or antigen binding fragment provided herein comprises a VH comprising the amino acid sequence of SEQ ID NO:260, and a VL comprising the amino acid sequence of SEQ ID NO:261. In some embodiments, the antibody or antigen binding fragment provided herein comprises a VH comprising the amino acid sequence of SEQ ID NO:262, and a LCDR1, an LCDR2, and an LCDR3 as set forth in SEQ ID NO:263. In some embodiments, the antibody or antigen binding fragment provided herein comprises a VH comprising the amino acid sequence of SEQ ID NO:264, and a VL comprising the amino acid sequence of SEQ ID NO:265. In some embodiments, the antibody or antigen binding fragment provided herein comprises a VH comprising the amino acid sequence of SEQ ID NO:268, and a VL comprising the amino acid sequence of SEQ ID NO:269. In some embodiments, the antibody or antigen binding fragment provided herein comprises a VH comprising the amino acid sequence of SEQ ID NO:786, and a VL comprising the amino acid sequence of SEQ ID NO:787. In some embodiments, the antibody or antigen binding fragment provided herein comprises a VH comprising the amino acid sequence of SEQ ID NO:788, and a VL comprising the amino acid sequence of SEQ ID NO:789. In some embodiments, the antibody or antigen binding fragment provided herein comprises a VH comprising the amino acid sequence of SEQ ID NO:790, and a VL comprising the amino acid sequence of SEQ ID NO:791. In some embodiments, the antibody or antigen binding fragment provided herein comprises a VH comprising the amino acid sequence of SEQ ID NO:792, and a VL comprising the amino acid sequence of SEQ ID NO:793. In some embodiments, the antibody or antigen binding fragment provided herein comprises a VH comprising the amino acid sequence of SEQ ID NO:798, and a VL comprising the amino acid sequence of SEQ ID NO:799. In some embodiments, the antibody or antigen binding fragment provided herein comprises a VH comprising the amino acid sequence of SEQ ID NO:806, and a VL comprising the amino acid sequence of SEQ ID NO:807. In some embodiments, the antibody or antigen binding fragment provided herein comprises a VH comprising the amino acid sequence of SEQ ID NO:808, and a VL comprising the amino acid sequence of SEQ ID NO:809. In some embodiments, the antibody or antigen binding fragment provided herein comprises a VH comprising the amino acid sequence of SEQ ID NO:810, and a VL comprising the amino acid sequence of SEQ ID NO:811. In some embodiments, the antibody or antigen binding fragment provided herein comprises a VH comprising the amino acid sequence of SEQ ID NO:812, and a VL comprising the amino acid sequence of SEQ ID NO:813. In some embodiments, the antibody or antigen binding fragment provided herein comprises a VH comprising the amino acid sequence of SEQ ID NO:814, and a VL comprising the amino acid sequence of SEQ ID NO:815. In some embodiments, the antibody or antigen binding fragment provided herein comprises a VH comprising the amino acid sequence of SEQ ID NO:816, and a VL comprising the amino acid sequence of SEQ ID NO:817. In some embodiments, the antibody or antigen binding fragment provided herein comprises a VH comprising the amino acid sequence of SEQ ID NO:818, and a VL comprising the amino acid sequence of SEQ ID NO:819. In some embodiments, the antibody or antigen binding fragment provided herein comprises a VH comprising the amino acid sequence of SEQ ID NO:820, and a VL comprising the amino acid sequence of SEQ ID NO:821. In some embodiments, the antibody or antigen binding fragment provided herein comprises a VH comprising the amino acid sequence of SEQ ID NO:822, and a VL comprising the amino acid sequence of SEQ ID NO:823. In some embodiments, the antibody or antigen binding fragment provided herein comprises a VH comprising the amino acid sequence of SEQ ID NO:824, and a VL comprising the amino acid sequence of SEQ ID NO:825. In some embodiments, the antibody or antigen binding fragment provided herein comprises a VH comprising the amino acid sequence of SEQ ID NO:826, and a VL comprising the amino acid sequence of SEQ ID NO:827. In some embodiments, the antibody or antigen binding fragment provided herein comprises a VH comprising the amino acid sequence of SEQ ID NO:828, and a VL comprising the amino acid sequence of SEQ ID NO:829. In some embodiments, the antibody or antigen binding fragment provided herein comprises a VH comprising the amino acid sequence of SEQ ID NO:830, and a VL comprising the amino acid sequence of SEQ ID NO:831. In some embodiments, the antibody or antigen binding fragment provided herein comprises a VH comprising the amino acid sequence of SEQ ID NO:832, and a VL comprising the amino acid sequence of SEQ ID NO:833. In some embodiments, the antibody or antigen binding fragment provided herein comprises a VH comprising the amino acid sequence of SEQ ID NO:836, and a VL comprising the amino acid sequence of SEQ ID NO:837. In some embodiments, the antibody or antigen binding fragment provided herein comprises a VH comprising the amino acid sequence of SEQ ID NO:838, and a VL comprising the amino acid sequence of SEQ ID NO:839. In some embodiments, the antibody or antigen binding fragment provided herein comprises a VH comprising the amino acid sequence of SEQ ID NO:840, and a VL comprising the amino acid sequence of SEQ ID NO:841. In some embodiments, the antibody or antigen binding fragment provided herein comprises a VH comprising the amino acid sequence of SEQ ID NO: 842, and a VL comprising the amino acid sequence of SEQ ID NO:843. In some embodiments, the antibody or antigen binding fragment provided herein comprises a VH comprising the amino acid sequence of SEQ ID NO:846, and a VL comprising the amino acid sequence of SEQ ID NO:847. In some embodiments, the antibody or antigen binding fragment provided herein comprises a VH comprising the amino acid sequence of SEQ ID NO:848, and a VL comprising the amino acid sequence of SEQ ID NO:849. In some embodiments, the antibody or antigen binding fragment provided herein comprises a VH comprising the amino acid sequence of SEQ ID NO:850, and a VL comprising the amino acid sequence of SEQ ID NO:851. In some embodiments, the antibody or antigen binding fragment provided herein comprises a VH comprising the amino acid sequence of SEQ ID NO:852, and a VL comprising the amino acid sequence of SEQ ID NO:853. In some embodiments, the antibody or antigen binding fragment provided herein comprises a VH comprising the amino acid sequence of SEQ ID NO:854, and a VL comprising the amino acid sequence of SEQ ID NO:855. In some embodiments, the antibody or antigen binding fragment provided herein comprises a VH comprising the amino acid sequence of SEQ ID NO:856, and a VL comprising the amino acid sequence of SEQ ID NO:857. In some embodiments, the antibody or antigen binding fragment provided herein comprises a VH comprising the amino acid sequence of SEQ ID NO:858, and a VL comprising the amino acid sequence of SEQ ID NO:859. In some embodiments, the antibody or antigen binding fragment provided herein comprises a VH comprising the amino acid sequence of SEQ ID NO:860, and a VL comprising the amino acid sequence of SEQ ID NO:861. In some embodiments, the antibody or antigen binding fragment provided herein comprises a VH comprising the amino acid sequence of SEQ ID NO:862, and a VL comprising the amino acid sequence of SEQ ID NO:863. In some embodiments, the antibody or antigen binding fragment provided herein comprises a VH comprising the amino acid sequence of SEQ ID NO:864, and a VL comprising the amino acid sequence of SEQ ID NO:865. In some embodiments, the antibody or antigen binding fragment provided herein comprises a VH comprising the amino acid sequence of SEQ ID NO:866, and a VL comprising the amino acid sequence of SEQ ID NO:867. In some embodiments, the antibody or antigen binding fragment provided herein comprises a VH comprising the amino acid sequence of SEQ ID NO:868, and a VL comprising the amino acid sequence of SEQ ID NO:869. In some embodiments, the antibody or antigen binding fragment provided herein comprises a VH comprising the amino acid sequence of SEQ ID NO:870, and a VL comprising the amino acid sequence of SEQ ID NO:871. In some embodiments, the antibody or antigen binding fragment provided herein comprises a VH comprising the amino acid sequence of SEQ ID NO:872, and a VL comprising the amino acid sequence of SEQ ID NO:873. In some embodiments, the antibody or antigen binding fragment provided herein comprises a VH comprising the amino acid sequence of SEQ ID NO:874, and a VL comprising the amino acid sequence of SEQ ID NO:875. In some embodiments, the antibody or antigen binding fragment provided herein comprises a VH comprising the amino acid sequence of SEQ ID NO:876, and a VL comprising the amino acid sequence of SEQ ID NO:877. In some embodiments, the antibody or antigen binding fragment provided herein comprises a VH comprising the amino acid sequence of SEQ ID NO:878, and a VL comprising the amino acid sequence of SEQ ID NO:879. In some embodiments, the antibody or antigen binding fragment provided herein comprises a VH comprising the amino acid sequence of SEQ ID NO:880, and a VL comprising the amino acid sequence of SEQ ID NO:881. In some embodiments, the antibody or antigen binding fragment provided herein comprises a VH comprising the amino acid sequence of SEQ ID NO:882, and a VL comprising the amino acid sequence of SEQ ID NO:883. In some embodiments, the antibody or antigen binding fragment provided herein comprises a VH comprising the amino acid sequence of SEQ ID NO:884, and a VL comprising the amino acid sequence of SEQ ID NO:885. In some embodiments, the antibody or antigen binding fragment provided herein comprises a VH comprising the amino acid sequence of SEQ ID NO:886, and a VL comprising the amino acid sequence of SEQ ID NO:887. In some embodiments, the antibody or antigen binding fragment provided herein comprises a VH comprising the amino acid sequence of SEQ ID NO:888, and a VL comprising the amino acid sequence of SEQ ID NO:889. In some embodiments, the antibody or antigen binding fragment provided herein comprises a VH comprising the amino acid sequence of SEQ ID NO:890, and a VL comprising the amino acid sequence of SEQ ID NO:891.

In certain embodiments, an antibody described herein or an antigen binding fragment thereof comprises amino acid sequences with certain percent identity relative to any antibody provided herein, for example, those described in the Examples providedbelow, including as set out in Tables 4-9.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, the antibody provided herein contains substitutions (e.g., conservative substitutions), insertions, or deletions relative to the reference sequence, but the anti-IL-23R antibody comprising that sequence retains the ability to bind to IL-23R. In some embodiments, the antibody provided herein contains substitutions (e.g., conservative substitutions), insertions, or deletions relative to the reference sequence, but the anti-IL-23R antibody comprising that sequence retains the ability to bind to IL-23R ECD. In some embodiments, a total of 1 to 10 amino acids have been substituted, inserted and/or deleted in a reference amino acid sequence. In some embodiments, a total of 1 to 10 amino acids have been substituted in a reference amino acid sequence. In some embodiments, substitutions, insertions, or deletions occur in regions outside the CDRs (i.e., in the FRs). In some embodiments, substitutions occur in regions outside the CDRs (i.e., in the FRs). In some embodiments, the anti-IL-23R antibody provided herein includes post-translational modifications of a reference sequence.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:138, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:139. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:142, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:143. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:144, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:145. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:148, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:149. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 150, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:151. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:152, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:153. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:154, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:155. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 156, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:157. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:158, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:159. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:160, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:161. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 162, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:163. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:164, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:165. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:166, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:167. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 168, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:169. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:170, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:171. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:172, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:173. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 174, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:175. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:176, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:177. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:178, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:179. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 180, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:181. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:182, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:183. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:186, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:187. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 188, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:189. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:190, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:191. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:192, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:193. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 194, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:195. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:196, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:197. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:198, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:199. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:200, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:201. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:202, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:203. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:204, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:205. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:206, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:207. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:208, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:209. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:210, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:211. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:212, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:213. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:214, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:215. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:216, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:217. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:218, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:219. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:220, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:221. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:222, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:223. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:226, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:227. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:228, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:229. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:230, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:231. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:232, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:233. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:234, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:235. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:236, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:237. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:238, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:239. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:240, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:241. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:246, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:247. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:248, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:249. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:250, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:251. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:252, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:253. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:254, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:255. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:256, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:257. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:258, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:259. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:260, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:261. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:262, and a LCDR1, an LCDR2, and an LCDR3 as set forth in SEQ ID NO:263. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:264, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:265. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:268, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:269. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:786, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:787. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:788, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:789. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:790, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:791. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:792, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:793. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:798, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:799. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:806, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:807. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:808, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:809. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:810, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:811. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:812, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:813. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:814, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:815. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:816, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:817. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:818, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:819. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:820, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:821. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:822, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:823. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:824, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:825. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:826, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:827. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:828, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:829. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:830, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:831. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:832, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:833. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:836, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:837. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:838, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:839. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:840, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:841. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 842, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:843. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:846, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:847. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:848, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:849. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:850, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:851. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:852, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:853. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:854, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:855. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:856, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:857. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:858, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:859. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:860, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:861. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:862, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:863. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:864, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:865. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:866, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:867. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:868, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:869. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:870, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:871. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:872, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:873. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:874, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:875. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:876, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:877. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:878, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:879. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:880, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:881. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:882, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:883. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:884, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:885. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:886, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:887. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:888, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:889. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:890, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:891. In all the embodiments described above, the antibodies bind to IL-23R.

In some embodiments, functional epitopes can be mapped, e.g., by combinatorial alanine scanning, to identify amino acids in the IL-23R protein that are necessary for interaction with anti-IL-23R antibodies provided herein. In some embodiments, conformational and crystal structure of anti-IL-23R antibody bound to IL-23R may be employed to identify the epitopes. In some embodiments, the present disclosure provides an antibody that specifically binds to the same epitope as any of the anti-IL-23R antibodies provided herein. For example, in some embodiments, the antibody or antigen binding fragment provided herein binds to the same epitope as an anti-IL-23R antibody comprising a VH comprising the amino acid sequence of SEQ ID NO:138, and a VL comprising the amino acid sequence of SEQ ID NO:139. In some embodiments, the antibody or antigen binding fragment provided herein binds to the same epitope as an anti-IL-23R antibody comprising a VH comprising the amino acid sequence of SEQ ID NO:142, and a VL comprising the amino acid sequence of SEQ ID NO:143. In some embodiments, the antibody or antigen binding fragment provided herein binds to the same epitope as an anti-IL-23R antibody comprising a VH comprising the amino acid sequence of SEQ ID NO:144, and a VL comprising the amino acid sequence of SEQ ID NO:145. In some embodiments, the antibody or antigen binding fragment provided herein binds to the same epitope as an anti-IL-23R antibody comprising a VH comprising the amino acid sequence of SEQ ID NO:148, and a VL comprising the amino acid sequence of SEQ ID NO:149. In some embodiments, the antibody or antigen binding fragment provided herein binds to the same epitope as an anti-IL-23R antibody comprising a VH comprising the amino acid sequence of SEQ ID NO:150, and a VL comprising the amino acid sequence of SEQ ID NO:151. In some embodiments, the antibody or antigen binding fragment provided herein binds to the same epitope as an anti-IL-23R antibody comprising a VH comprising the amino acid sequence of SEQ ID NO:152, and a VL comprising the amino acid sequence of SEQ ID NO:153. In some embodiments, the antibody or antigen binding fragment provided herein binds to the same epitope as an anti-IL-23R antibody comprising a VH comprising the amino acid sequence of SEQ ID NO:154, and a VL comprising the amino acid sequence of SEQ ID NO:155. In some embodiments, the antibody or antigen binding fragment provided herein binds to the same epitope as an anti-IL-23R antibody comprising a VH comprising the amino acid sequence of SEQ ID NO:156, and a VL comprising the amino acid sequence of SEQ ID NO:157. In some embodiments, the antibody or antigen binding fragment provided herein binds to the same epitope as an anti-IL-23R antibody comprising a VH comprising the amino acid sequence of SEQ ID NO:158, and a VL comprising the amino acid sequence of SEQ ID NO:159. In some embodiments, the antibody or antigen binding fragment provided herein binds to the same epitope as an anti-IL-23R antibody comprising a VH comprising the amino acid sequence of SEQ ID NO:160, and a VL comprising the amino acid sequence of SEQ ID NO:161. In some embodiments, the antibody or antigen binding fragment provided herein binds to the same epitope as an anti-IL-23R antibody comprising a VH comprising the amino acid sequence of SEQ ID NO:162, and a VL comprising the amino acid sequence of SEQ ID NO:163. In some embodiments, the antibody or antigen binding fragment provided herein binds to the same epitope as an anti-IL-23R antibody comprising a VH comprising the amino acid sequence of SEQ ID NO:164, and a VL comprising the amino acid sequence of SEQ ID NO:165. In some embodiments, the antibody or antigen binding fragment provided herein binds to the same epitope as an anti-IL-23R antibody comprising a VH comprising the amino acid sequence of SEQ ID NO:166, and a VL comprising the amino acid sequence of SEQ ID NO:167. In some embodiments, the antibody or antigen binding fragment provided herein binds to the same epitope as an anti-IL-23R antibody comprising a VH comprising the amino acid sequence of SEQ ID NO:168, and a VL comprising the amino acid sequence of SEQ ID NO:169. In some embodiments, the antibody or antigen binding fragment provided herein binds to the same epitope as an anti-IL-23R antibody comprising a VH comprising the amino acid sequence of SEQ ID NO:170, and a VL comprising the amino acid sequence of SEQ ID NO:171. In some embodiments, the antibody or antigen binding fragment provided herein binds to the same epitope as an anti-IL-23R antibody comprising a VH comprising the amino acid sequence of SEQ ID NO:172, and a VL comprising the amino acid sequence of SEQ ID NO:173. In some embodiments, the antibody or antigen binding fragment provided herein binds to the same epitope as an anti-IL-23R antibody comprising a VH comprising the amino acid sequence of SEQ ID NO:174, and a VL comprising the amino acid sequence of SEQ ID NO:175. In some embodiments, the antibody or antigen binding fragment provided herein binds to the same epitope as an anti-IL-23R antibody comprising a VH comprising the amino acid sequence of SEQ ID NO:176, and a VL comprising the amino acid sequence of SEQ ID NO:177. In some embodiments, the antibody or antigen binding fragment provided herein binds to the same epitope as an anti-IL-23R antibody comprising a VH comprising the amino acid sequence of SEQ ID NO:178, and a VL comprising the amino acid sequence of SEQ ID NO:179. In some embodiments, the antibody or antigen binding fragment provided herein binds to the same epitope as an anti-IL-23R antibody comprising a VH comprising the amino acid sequence of SEQ ID NO:180, and a VL comprising the amino acid sequence of SEQ ID NO:181. In some embodiments, the antibody or antigen binding fragment provided herein binds to the same epitope as an anti-IL-23R antibody comprising a VH comprising the amino acid sequence of SEQ ID NO:182, and a VL comprising the amino acid sequence of SEQ ID NO:183. In some embodiments, the antibody or antigen binding fragment provided herein binds to the same epitope as an anti-IL-23R antibody comprising a VH comprising the amino acid sequence of SEQ ID NO:186, and a VL comprising the amino acid sequence of SEQ ID NO:187. In some embodiments, the antibody or antigen binding fragment provided herein binds to the same epitope as an anti-IL-23R antibody comprising a VH comprising the amino acid sequence of SEQ ID NO:188, and a VL comprising the amino acid sequence of SEQ ID NO:189. In some embodiments, the antibody or antigen binding fragment provided herein binds to the same epitope as an anti-IL-23R antibody comprising a VH comprising the amino acid sequence of SEQ ID NO:190, and a VL comprising the amino acid sequence of SEQ ID NO:191. In some embodiments, the antibody or antigen binding fragment provided herein binds to the same epitope as an anti-IL-23R antibody comprising a VH comprising the amino acid sequence of SEQ ID NO:192, and a VL comprising the amino acid sequence of SEQ ID NO:193. In some embodiments, the antibody or antigen binding fragment provided herein binds to the same epitope as an anti-IL-23R antibody comprising a VH comprising the amino acid sequence of SEQ ID NO:194, and a VL comprising the amino acid sequence of SEQ ID NO:195. In some embodiments, the antibody or antigen binding fragment provided herein binds to the same epitope as an anti-IL-23R antibody comprising a VH comprising the amino acid sequence of SEQ ID NO:196, and a VL comprising the amino acid sequence of SEQ ID NO:197. In some embodiments, the antibody or antigen binding fragment provided herein binds to the same epitope as an anti-IL-23R antibody comprising a VH comprising the amino acid sequence of SEQ ID NO:198, and a VL comprising the amino acid sequence of SEQ ID NO:199. In some embodiments, the antibody or antigen binding fragment provided herein binds to the same epitope as an anti-IL-23R antibody comprising a VH comprising the amino acid sequence of SEQ ID NO:200, and a VL comprising the amino acid sequence of SEQ ID NO:201. In some embodiments, the antibody or antigen binding fragment provided herein binds to the same epitope as an anti-IL-23R antibody comprising a VH comprising the amino acid sequence of SEQ ID NO:202, and a VL comprising the amino acid sequence of SEQ ID NO:203. In some embodiments, the antibody or antigen binding fragment provided herein binds to the same epitope as an anti-IL-23R antibody comprising a VH comprising the amino acid sequence of SEQ ID NO:204, and a VL comprising the amino acid sequence of SEQ ID NO:205. In some embodiments, the antibody or antigen binding fragment provided herein binds to the same epitope as an anti-IL-23R antibody comprising a VH comprising the amino acid sequence of SEQ ID NO:206, and a VL comprising the amino acid sequence of SEQ ID NO:207. In some embodiments, the antibody or antigen binding fragment provided herein binds to the same epitope as an anti-IL-23R antibody comprising a VH comprising the amino acid sequence of SEQ ID NO:208, and a VL comprising the amino acid sequence of SEQ ID NO:209. In some embodiments, the antibody or antigen binding fragment provided herein binds to the same epitope as an anti-IL-23R antibody comprising a VH comprising the amino acid sequence of SEQ ID NO:210, and a VL comprising the amino acid sequence of SEQ ID NO:211. In some embodiments, the antibody or antigen binding fragment provided herein binds to the same epitope as an anti-IL-23R antibody comprising a VH comprising the amino acid sequence of SEQ ID NO:212, and a VL comprising the amino acid sequence of SEQ ID NO:213. In some embodiments, the antibody or antigen binding fragment provided herein binds to the same epitope as an anti-IL-23R antibody comprising a VH comprising the amino acid sequence of SEQ ID NO:214, and a VL comprising the amino acid sequence of SEQ ID NO:215. In some embodiments, the antibody or antigen binding fragment provided herein binds to the same epitope as an anti-IL-23R antibody comprising a VH comprising the amino acid sequence of SEQ ID NO:216, and a VL comprising the amino acid sequence of SEQ ID NO:217. In some embodiments, the antibody or antigen binding fragment provided herein binds to the same epitope as an anti-IL-23R antibody comprising a VH comprising the amino acid sequence of SEQ ID NO:218, and a VL comprising the amino acid sequence of SEQ ID NO:219. In some embodiments, the antibody or antigen binding fragment provided herein binds to the same epitope as an anti-IL-23R antibody comprising a VH comprising the amino acid sequence of SEQ ID NO:220, and a VL comprising the amino acid sequence of SEQ ID NO:221. In some embodiments, the antibody or antigen binding fragment provided herein binds to the same epitope as an anti-IL-23R antibody comprising a VH comprising the amino acid sequence of SEQ ID NO:222, and a VL comprising the amino acid sequence of SEQ ID NO:223. In some embodiments, the antibody or antigen binding fragment provided herein binds to the same epitope as an anti-IL-23R antibody comprising a VH comprising the amino acid sequence of SEQ ID NO:226, and a VL comprising the amino acid sequence of SEQ ID NO:227. In some embodiments, the antibody or antigen binding fragment provided herein binds to the same epitope as an anti-IL-23R antibody comprising a VH comprising the amino acid sequence of SEQ ID NO:228, and a VL comprising the amino acid sequence of SEQ ID NO:229. In some embodiments, the antibody or antigen binding fragment provided herein binds to the same epitope as an anti-IL-23R antibody comprising a VH comprising the amino acid sequence of SEQ ID NO:230, and a VL comprising the amino acid sequence of SEQ ID NO:231. In some embodiments, the antibody or antigen binding fragment provided herein binds to the same epitope as an anti-IL-23R antibody comprising a VH comprising the amino acid sequence of SEQ ID NO:232, and a VL comprising the amino acid sequence of SEQ ID NO:233. In some embodiments, the antibody or antigen binding fragment provided herein binds to the same epitope as an anti-IL-23R antibody comprising a VH comprising the amino acid sequence of SEQ ID NO:234, and a VL comprising the amino acid sequence of SEQ ID NO:235. In some embodiments, the antibody or antigen binding fragment provided herein binds to the same epitope as an anti-IL-23R antibody comprising a VH comprising the amino acid sequence of SEQ ID NO:236, and a VL comprising the amino acid sequence of SEQ ID NO:237. In some embodiments, the antibody or antigen binding fragment provided herein binds to the same epitope as an anti-IL-23R antibody comprising a VH comprising the amino acid sequence of SEQ ID NO:238, and a VL comprising the amino acid sequence of SEQ ID NO:239. In some embodiments, the antibody or antigen binding fragment provided herein binds to the same epitope as an anti-IL-23R antibody comprising a VH comprising the amino acid sequence of SEQ ID NO:240, and a VL comprising the amino acid sequence of SEQ ID NO:241. In some embodiments, the antibody or antigen binding fragment provided herein binds to the same epitope as an anti-IL-23R antibody comprising a VH comprising the amino acid sequence of SEQ ID NO:246, and a VL comprising the amino acid sequence of SEQ ID NO:247. In some embodiments, the antibody or antigen binding fragment provided herein binds to the same epitope as an anti-IL-23R antibody comprising a VH comprising the amino acid sequence of SEQ ID NO:248, and a VL comprising the amino acid sequence of SEQ ID NO:249. In some embodiments, the antibody or antigen binding fragment provided herein binds to the same epitope as an anti-IL-23R antibody comprising a VH comprising the amino acid sequence of SEQ ID NO:250, and a VL comprising the amino acid sequence of SEQ ID NO:251. In some embodiments, the antibody or antigen binding fragment provided herein binds to the same epitope as an anti-IL-23R antibody comprising a VH comprising the amino acid sequence of SEQ ID NO:252, and a VL comprising the amino acid sequence of SEQ ID NO:253. In some embodiments, the antibody or antigen binding fragment provided herein binds to the same epitope as an anti-IL-23R antibody comprising a VH comprising the amino acid sequence of SEQ ID NO:254, and a VL comprising the amino acid sequence of SEQ ID NO:255. In some embodiments, the antibody or antigen binding fragment provided herein binds to the same epitope as an anti-IL-23R antibody comprising a VH comprising the amino acid sequence of SEQ ID NO:256, and a VL comprising the amino acid sequence of SEQ ID NO:257. In some embodiments, the antibody or antigen binding fragment provided herein binds to the same epitope as an anti-IL-23R antibody comprising a VH comprising the amino acid sequence of SEQ ID NO:258, and a VL comprising the amino acid sequence of SEQ ID NO:259. In some embodiments, the antibody or antigen binding fragment provided herein binds to the same epitope as an anti-IL-23R antibody comprising a VH comprising the amino acid sequence of SEQ ID NO:260, and a VL comprising the amino acid sequence of SEQ ID NO:261. In some embodiments, the antibody or antigen binding fragment provided herein binds to the same epitope as an anti-IL-23R antibody comprising a VH comprising the amino acid sequence of SEQ ID NO:262, and a VL comprising the amino acid sequence of SEQ ID NO:263. In some embodiments, the antibody or antigen binding fragment provided herein binds to the same epitope as an anti-IL-23R antibody comprising a VH comprising the amino acid sequence of SEQ ID NO:264, and a VL comprising the amino acid sequence of SEQ ID NO:265. In some embodiments, the antibody or antigen binding fragment provided herein binds to the same epitope as an anti-IL-23R antibody comprising a VH comprising the amino acid sequence of SEQ ID NO:268, and a VL comprising the amino acid sequence of SEQ ID NO:269. In some embodiments, the antibody or antigen binding fragment provided herein binds to the same epitope as an anti-IL-23R antibody comprising a VH comprising the amino acid sequence of SEQ ID NO:786, and a VL comprising the amino acid sequence of SEQ ID NO:787. In some embodiments, the antibody or antigen binding fragment provided herein binds to the same epitope as an anti-IL-23R antibody comprising a VH comprising the amino acid sequence of SEQ ID NO:788, and a VL comprising the amino acid sequence of SEQ ID NO:789. In some embodiments, the antibody or antigen binding fragment provided herein binds to the same epitope as an anti-IL-23R antibody comprising a VH comprising the amino acid sequence of SEQ ID NO:790, and a VL comprising the amino acid sequence of SEQ ID NO:791. In some embodiments, the antibody or antigen binding fragment provided herein binds to the same epitope as an anti-IL-23R antibody comprising a VH comprising the amino acid sequence of SEQ ID NO:792, and a VL comprising the amino acid sequence of SEQ ID NO:793. In some embodiments, the antibody or antigen binding fragment provided herein binds to the same epitope as an anti-IL-23R antibody comprising a VH comprising the amino acid sequence of SEQ ID NO:798, and a VL comprising the amino acid sequence of SEQ ID NO:799. In some embodiments, the antibody or antigen binding fragment provided herein binds to the same epitope as an anti-IL-23R antibody comprising a VH comprising the amino acid sequence of SEQ ID NO:806, and a VL comprising the amino acid sequence of SEQ ID NO:807. In some embodiments, the antibody or antigen binding fragment provided herein binds to the same epitope as an anti-IL-23R antibody comprising a VH comprising the amino acid sequence of SEQ ID NO:808, and a VL comprising the amino acid sequence of SEQ ID NO:809. In some embodiments, the antibody or antigen binding fragment provided herein binds to the same epitope as an anti-IL-23R antibody comprising a VH comprising the amino acid sequence of SEQ ID NO:810, and a VL comprising the amino acid sequence of SEQ ID NO:811. In some embodiments, the antibody or antigen binding fragment provided herein binds to the same epitope as an anti-IL-23R antibody comprising a VH comprising the amino acid sequence of SEQ ID NO:812, and a VL comprising the amino acid sequence of SEQ ID NO:813. In some embodiments, the antibody or antigen binding fragment provided herein binds to the same epitope as an anti-IL-23R antibody comprising a VH comprising the amino acid sequence of SEQ ID NO:814, and a VL comprising the amino acid sequence of SEQ ID NO:815. In some embodiments, the antibody or antigen binding fragment provided herein binds to the same epitope as an anti-IL-23R antibody comprising a VH comprising the amino acid sequence of SEQ ID NO:816, and a VL comprising the amino acid sequence of SEQ ID NO:817. In some embodiments, the antibody or antigen binding fragment provided herein binds to the same epitope as an anti-IL-23R antibody comprising a VH comprising the amino acid sequence of SEQ ID NO:818, and a VL comprising the amino acid sequence of SEQ ID NO:819. In some embodiments, the antibody or antigen binding fragment provided herein binds to the same epitope as an anti-IL-23R antibody comprising a VH comprising the amino acid sequence of SEQ ID NO:820, and a VL comprising the amino acid sequence of SEQ ID NO:821. In some embodiments, the antibody or antigen binding fragment provided herein binds to the same epitope as an anti-IL-23R antibody comprising a VH comprising the amino acid sequence of SEQ ID NO:822, and a VL comprising the amino acid sequence of SEQ ID NO:823. In some embodiments, the antibody or antigen binding fragment provided herein binds to the same epitope as an anti-IL-23R antibody comprising a VH comprising the amino acid sequence of SEQ ID NO:824, and a VL comprising the amino acid sequence of SEQ ID NO:825. In some embodiments, the antibody or antigen binding fragment provided herein binds to the same epitope as an anti-IL-23R antibody comprising a VH comprising the amino acid sequence of SEQ ID NO:826, and a VL comprising the amino acid sequence of SEQ ID NO:827. In some embodiments, the antibody or antigen binding fragment provided herein binds to the same epitope as an anti-IL-23R antibody comprising a VH comprising the amino acid sequence of SEQ ID NO:828, and a VL comprising the amino acid sequence of SEQ ID NO:829. In some embodiments, the antibody or antigen binding fragment provided herein binds to the same epitope as an anti-IL-23R antibody comprising a VH comprising the amino acid sequence of SEQ ID NO:830, and a VL comprising the amino acid sequence of SEQ ID NO:831. In some embodiments, the antibody or antigen binding fragment provided herein binds to the same epitope as an anti-IL-23R antibody comprising a VH comprising the amino acid sequence of SEQ ID NO:832, and a VL comprising the amino acid sequence of SEQ ID NO:833. In some embodiments, the antibody or antigen binding fragment provided herein binds to the same epitope as an anti-IL-23R antibody comprising a VH comprising the amino acid sequence of SEQ ID NO:836, and a VL comprising the amino acid sequence of SEQ ID NO:837. In some embodiments, the antibody or antigen binding fragment provided herein binds to the same epitope as an anti-IL-23R antibody comprising a VH comprising the amino acid sequence of SEQ ID NO:838, and a VL comprising the amino acid sequence of SEQ ID NO:839. In some embodiments, the antibody or antigen binding fragment provided herein binds to the same epitope as an anti-IL-23R antibody comprising a VH comprising the amino acid sequence of SEQ ID NO:840, and a VL comprising the amino acid sequence of SEQ ID NO:841. In some embodiments, the antibody or antigen binding fragment provided herein binds to the same epitope as an anti-IL-23R antibody comprising a VH comprising the amino acid sequence of SEQ ID NO: 842, and a VL comprising the amino acid sequence of SEQ ID NO:843. In some embodiments, the antibody or antigen binding fragment provided herein binds to the same epitope as an anti-IL-23R antibody comprising a VH comprising the amino acid sequence of SEQ ID NO:846, and a VL comprising the amino acid sequence of SEQ ID NO:847. In some embodiments, the antibody or antigen binding fragment provided herein binds to the same epitope as an anti-IL-23R antibody comprising a VH comprising the amino acid sequence of SEQ ID NO:848, and a VL comprising the amino acid sequence of SEQ ID NO:849. In some embodiments, the antibody or antigen binding fragment provided herein binds to the same epitope as an anti-IL-23R antibody comprising a VH comprising the amino acid sequence of SEQ ID NO:850, and a VL comprising the amino acid sequence of SEQ ID NO:851. In some embodiments, the antibody or antigen binding fragment provided herein binds to the same epitope as an anti-IL-23R antibody comprising a VH comprising the amino acid sequence of SEQ ID NO:852, and a VL comprising the amino acid sequence of SEQ ID NO:853. In some embodiments, the antibody or antigen binding fragment provided herein binds to the same epitope as an anti-IL-23R antibody comprising a VH comprising the amino acid sequence of SEQ ID NO:854, and a VL comprising the amino acid sequence of SEQ ID NO:855. In some embodiments, the antibody or antigen binding fragment provided herein binds to the same epitope as an anti-IL-23R antibody comprising a VH comprising the amino acid sequence of SEQ ID NO:856, and a VL comprising the amino acid sequence of SEQ ID NO:857. In some embodiments, the antibody or antigen binding fragment provided herein binds to the same epitope as an anti-IL-23R antibody comprising a VH comprising the amino acid sequence of SEQ ID NO:858, and a VL comprising the amino acid sequence of SEQ ID NO:859. In some embodiments, the antibody or antigen binding fragment provided herein binds to the same epitope as an anti-IL-23R antibody comprising a VH comprising the amino acid sequence of SEQ ID NO:860, and a VL comprising the amino acid sequence of SEQ ID NO:861. In some embodiments, the antibody or antigen binding fragment provided herein binds to the same epitope as an anti-IL-23R antibody comprising a VH comprising the amino acid sequence of SEQ ID NO:862, and a VL comprising the amino acid sequence of SEQ ID NO:863. In some embodiments, the antibody or antigen binding fragment provided herein binds to the same epitope as an anti-IL-23R antibody comprising a VH comprising the amino acid sequence of SEQ ID NO:864, and a VL comprising the amino acid sequence of SEQ ID NO:865. In some embodiments, the antibody or antigen binding fragment provided herein binds to the same epitope as an anti-IL-23R antibody comprising a VH comprising the amino acid sequence of SEQ ID NO:866, and a VL comprising the amino acid sequence of SEQ ID NO:867. In some embodiments, the antibody or antigen binding fragment provided herein binds to the same epitope as an anti-IL-23R antibody comprising a VH comprising the amino acid sequence of SEQ ID NO:868, and a VL comprising the amino acid sequence of SEQ ID NO:869. In some embodiments, the antibody or antigen binding fragment provided herein binds to the same epitope as an anti-IL-23R antibody comprising a VH comprising the amino acid sequence of SEQ ID NO:870, and a VL comprising the amino acid sequence of SEQ ID NO:871. In some embodiments, the antibody or antigen binding fragment provided herein binds to the same epitope as an anti-IL-23R antibody comprising a VH comprising the amino acid sequence of SEQ ID NO:872, and a VL comprising the amino acid sequence of SEQ ID NO:873. In some embodiments, the antibody or antigen binding fragment provided herein binds to the same epitope as an anti-IL-23R antibody comprising a VH comprising the amino acid sequence of SEQ ID NO:874, and a VL comprising the amino acid sequence of SEQ ID NO:875. In some embodiments, the antibody or antigen binding fragment provided herein binds to the same epitope as an anti-IL-23R antibody comprising a VH comprising the amino acid sequence of SEQ ID NO:876, and a VL comprising the amino acid sequence of SEQ ID NO:877. In some embodiments, the antibody or antigen binding fragment provided herein binds to the same epitope as an anti-IL-23R antibody comprising a VH comprising the amino acid sequence of SEQ ID NO:878, and a VL comprising the amino acid sequence of SEQ ID NO:879. In some embodiments, the antibody or antigen binding fragment provided herein binds to the same epitope as an anti-IL-23R antibody comprising a VH comprising the amino acid sequence of SEQ ID NO:880, and a VL comprising the amino acid sequence of SEQ ID NO:881. In some embodiments, the antibody or antigen binding fragment provided herein binds to the same epitope as an anti-IL-23R antibody comprising a VH comprising the amino acid sequence of SEQ ID NO:882, and a VL comprising the amino acid sequence of SEQ ID NO:883. In some embodiments, the antibody or antigen binding fragment provided herein binds to the same epitope as an anti-IL-23R antibody comprising a VH comprising the amino acid sequence of SEQ ID NO:884, and a VL comprising the amino acid sequence of SEQ ID NO:885. In some embodiments, the antibody or antigen binding fragment provided herein binds to the same epitope as an anti-IL-23R antibody comprising a VH comprising the amino acid sequence of SEQ ID NO:886, and a VL comprising the amino acid sequence of SEQ ID NO:887. In some embodiments, the antibody or antigen binding fragment provided herein binds to the same epitope as an anti-IL-23R antibody comprising a VH comprising the amino acid sequence of SEQ ID NO:888, and a VL comprising the amino acid sequence of SEQ ID NO:889. In some embodiments, the antibody or antigen binding fragment provided herein binds to the same epitope as an anti-IL-23R antibody comprising a VH comprising the amino acid sequence of SEQ ID NO:890, and a VL comprising the amino acid sequence of SEQ ID NO:891.

In some embodiments, provided herein is an anti-IL-23R antibody, or antigen binding fragment thereof, that specifically binds to IL-23R competitively with any one of the anti-IL-23R antibodies described herein. In some embodiments, the antibody or antigen binding fragment provided herein specifically binds to IL-23R competitively with an anti-IL-23R antibody comprising a VH comprising the amino acid sequence of SEQ ID NO:138, and a VL comprising the amino acid sequence of SEQ ID NO:139. In some embodiments, the antibody or antigen binding fragment provided herein specifically binds to IL-23R competitively with an anti-IL-23R antibody comprising a VH comprising the amino acid sequence of SEQ ID NO: 142, and a VL comprising the amino acid sequence of SEQ ID NO:143. In some embodiments, the antibody or antigen binding fragment provided herein specifically binds to IL-23R competitively with an anti-IL-23R antibody comprising a VH comprising the amino acid sequence of SEQ ID NO: 144, and a VL comprising the amino acid sequence of SEQ ID NO:145. In some embodiments, the antibody or antigen binding fragment provided herein specifically binds to IL-23R competitively with an anti-IL-23R antibody comprising a VH comprising the amino acid sequence of SEQ ID NO: 148, and a VL comprising the amino acid sequence of SEQ ID NO:149. In some embodiments, the antibody or antigen binding fragment provided herein specifically binds to IL-23R competitively with an anti-IL-23R antibody comprising a VH comprising the amino acid sequence of SEQ ID NO: 150, and a VL comprising the amino acid sequence of SEQ ID NO:151. In some embodiments, the antibody or antigen binding fragment provided herein specifically binds to IL-23R competitively with an anti-IL-23R antibody comprising a VH comprising the amino acid sequence of SEQ ID NO: 152, and a VL comprising the amino acid sequence of SEQ ID NO:153. In some embodiments, the antibody or antigen binding fragment provided herein specifically binds to IL-23R competitively with an anti-IL-23R antibody comprising a VH comprising the amino acid sequence of SEQ ID NO: 154, and a VL comprising the amino acid sequence of SEQ ID NO:155. In some embodiments, the antibody or antigen binding fragment provided herein specifically binds to IL-23R competitively with an anti-IL-23R antibody comprising a VH comprising the amino acid sequence of SEQ ID NO: 156, and a VL comprising the amino acid sequence of SEQ ID NO:157. In some embodiments, the antibody or antigen binding fragment provided herein specifically binds to IL-23R competitively with an anti-IL-23R antibody comprising a VH comprising the amino acid sequence of SEQ ID NO: 158, and a VL comprising the amino acid sequence of SEQ ID NO:159. In some embodiments, the antibody or antigen binding fragment provided herein specifically binds to IL-23R competitively with an anti-IL-23R antibody comprising a VH comprising the amino acid sequence of SEQ ID NO: 160, and a VL comprising the amino acid sequence of SEQ ID NO:161. In some embodiments, the antibody or antigen binding fragment provided herein specifically binds to IL-23R competitively with an anti-IL-23R antibody comprising a VH comprising the amino acid sequence of SEQ ID NO: 162, and a VL comprising the amino acid sequence of SEQ ID NO:163. In some embodiments, the antibody or antigen binding fragment provided herein specifically binds to IL-23R competitively with an anti-IL-23R antibody comprising a VH comprising the amino acid sequence of SEQ ID NO: 164, and a VL comprising the amino acid sequence of SEQ ID NO:165. In some embodiments, the antibody or antigen binding fragment provided herein specifically binds to IL-23R competitively with an anti-IL-23R antibody comprising a VH comprising the amino acid sequence of SEQ ID NO: 166, and a VL comprising the amino acid sequence of SEQ ID NO:167. In some embodiments, the antibody or antigen binding fragment provided herein specifically binds to IL-23R competitively with an anti-IL-23R antibody comprising a VH comprising the amino acid sequence of SEQ ID NO: 168, and a VL comprising the amino acid sequence of SEQ ID NO:169. In some embodiments, the antibody or antigen binding fragment provided herein specifically binds to IL-23R competitively with an anti-IL-23R antibody comprising a VH comprising the amino acid sequence of SEQ ID NO: 170, and a VL comprising the amino acid sequence of SEQ ID NO:171. In some embodiments, the antibody or antigen binding fragment provided herein specifically binds to IL-23R competitively with an anti-IL-23R antibody comprising a VH comprising the amino acid sequence of SEQ ID NO: 172, and a VL comprising the amino acid sequence of SEQ ID NO:173. In some embodiments, the antibody or antigen binding fragment provided herein specifically binds to IL-23R competitively with an anti-IL-23R antibody comprising a VH comprising the amino acid sequence of SEQ ID NO: 174, and a VL comprising the amino acid sequence of SEQ ID NO:175. In some embodiments, the antibody or antigen binding fragment provided herein specifically binds to IL-23R competitively with an anti-IL-23R antibody comprising a VH comprising the amino acid sequence of SEQ ID NO: 176, and a VL comprising the amino acid sequence of SEQ ID NO:177. In some embodiments, the antibody or antigen binding fragment provided herein specifically binds to IL-23R competitively with an anti-IL-23R antibody comprising a VH comprising the amino acid sequence of SEQ ID NO: 178, and a VL comprising the amino acid sequence of SEQ ID NO:179. In some embodiments, the antibody or antigen binding fragment provided herein specifically binds to IL-23R competitively with an anti-IL-23R antibody comprising a VH comprising the amino acid sequence of SEQ ID NO: 180, and a VL comprising the amino acid sequence of SEQ ID NO:181. In some embodiments, the antibody or antigen binding fragment provided herein specifically binds to IL-23R competitively with an anti-IL-23R antibody comprising a VH comprising the amino acid sequence of SEQ ID NO: 182, and a VL comprising the amino acid sequence of SEQ ID NO:183. In some embodiments, the antibody or antigen binding fragment provided herein specifically binds to IL-23R competitively with an anti-IL-23R antibody comprising a VH comprising the amino acid sequence of SEQ ID NO: 186, and a VL comprising the amino acid sequence of SEQ ID NO:187. In some embodiments, the antibody or antigen binding fragment provided herein specifically binds to IL-23R competitively with an anti-IL-23R antibody comprising a VH comprising the amino acid sequence of SEQ ID NO: 188, and a VL comprising the amino acid sequence of SEQ ID NO:189. In some embodiments, the antibody or antigen binding fragment provided herein specifically binds to IL-23R competitively with an anti-IL-23R antibody comprising a VH comprising the amino acid sequence of SEQ ID NO: 190, and a VL comprising the amino acid sequence of SEQ ID NO:191. In some embodiments, the antibody or antigen binding fragment provided herein specifically binds to IL-23R competitively with an anti-IL-23R antibody comprising a VH comprising the amino acid sequence of SEQ ID NO: 192, and a VL comprising the amino acid sequence of SEQ ID NO:193. In some embodiments, the antibody or antigen binding fragment provided herein specifically binds to IL-23R competitively with an anti-IL-23R antibody comprising a VH comprising the amino acid sequence of SEQ ID NO: 194, and a VL comprising the amino acid sequence of SEQ ID NO:195. In some embodiments, the antibody or antigen binding fragment provided herein specifically binds to IL-23R competitively with an anti-IL-23R antibody comprising a VH comprising the amino acid sequence of SEQ ID NO: 196, and a VL comprising the amino acid sequence of SEQ ID NO:197. In some embodiments, the antibody or antigen binding fragment provided herein specifically binds to IL-23R competitively with an anti-IL-23R antibody comprising a VH comprising the amino acid sequence of SEQ ID NO: 198, and a VL comprising the amino acid sequence of SEQ ID NO:199. In some embodiments, the antibody or antigen binding fragment provided herein specifically binds to IL-23R competitively with an anti-IL-23R antibody comprising a VH comprising the amino acid sequence of SEQ ID NO:200, and a VL comprising the amino acid sequence of SEQ ID NO:201. In some embodiments, the antibody or antigen binding fragment provided herein specifically binds to IL-23R competitively with an anti-IL-23R antibody comprising a VH comprising the amino acid sequence of SEQ ID NO:202, and a VL comprising the amino acid sequence of SEQ ID NO:203. In some embodiments, the antibody or antigen binding fragment provided herein specifically binds to IL-23R competitively with an anti-IL-23R antibody comprising a VH comprising the amino acid sequence of SEQ ID NO:204, and a VL comprising the amino acid sequence of SEQ ID NO:205. In some embodiments, the antibody or antigen binding fragment provided herein specifically binds to IL-23R competitively with an anti-IL-23R antibody comprising a VH comprising the amino acid sequence of SEQ ID NO:206, and a VL comprising the amino acid sequence of SEQ ID NO:207. In some embodiments, the antibody or antigen binding fragment provided herein specifically binds to IL-23R competitively with an anti-IL-23R antibody comprising a VH comprising the amino acid sequence of SEQ ID NO:208, and a VL comprising the amino acid sequence of SEQ ID NO:209. In some embodiments, the antibody or antigen binding fragment provided herein specifically binds to IL-23R competitively with an anti-IL-23R antibody comprising a VH comprising the amino acid sequence of SEQ ID NO:210, and a VL comprising the amino acid sequence of SEQ ID NO:211. In some embodiments, the antibody or antigen binding fragment provided herein specifically binds to IL-23R competitively with an anti-IL-23R antibody comprising a VH comprising the amino acid sequence of SEQ ID NO:212, and a VL comprising the amino acid sequence of SEQ ID NO:213. In some embodiments, the antibody or antigen binding fragment provided herein specifically binds to IL-23R competitively with an anti-IL-23R antibody comprising a VH comprising the amino acid sequence of SEQ ID NO:214, and a VL comprising the amino acid sequence of SEQ ID NO:215. In some embodiments, the antibody or antigen binding fragment provided herein specifically binds to IL-23R competitively with an anti-IL-23R antibody comprising a VH comprising the amino acid sequence of SEQ ID NO:216, and a VL comprising the amino acid sequence of SEQ ID NO:217. In some embodiments, the antibody or antigen binding fragment provided herein specifically binds to IL-23R competitively with an anti-IL-23R antibody comprising a VH comprising the amino acid sequence of SEQ ID NO:218, and a VL comprising the amino acid sequence of SEQ ID NO:219. In some embodiments, the antibody or antigen binding fragment provided herein specifically binds to IL-23R competitively with an anti-IL-23R antibody comprising a VH comprising the amino acid sequence of SEQ ID NO:220, and a VL comprising the amino acid sequence of SEQ ID NO:221. In some embodiments, the antibody or antigen binding fragment provided herein specifically binds to IL-23R competitively with an anti-IL-23R antibody comprising a VH comprising the amino acid sequence of SEQ ID NO:222, and a VL comprising the amino acid sequence of SEQ ID NO:223. In some embodiments, the antibody or antigen binding fragment provided herein specifically binds to IL-23R competitively with an anti-IL-23R antibody comprising a VH comprising the amino acid sequence of SEQ ID NO:226, and a VL comprising the amino acid sequence of SEQ ID NO:227. In some embodiments, the antibody or antigen binding fragment provided herein specifically binds to IL-23R competitively with an anti-IL-23R antibody comprising a VH comprising the amino acid sequence of SEQ ID NO:228, and a VL comprising the amino acid sequence of SEQ ID NO:229. In some embodiments, the antibody or antigen binding fragment provided herein specifically binds to IL-23R competitively with an anti-IL-23R antibody comprising a VH comprising the amino acid sequence of SEQ ID NO:230, and a VL comprising the amino acid sequence of SEQ ID NO:231. In some embodiments, the antibody or antigen binding fragment provided herein specifically binds to IL-23R competitively with an anti-IL-23R antibody comprising a VH comprising the amino acid sequence of SEQ ID NO:232, and a VL comprising the amino acid sequence of SEQ ID NO:233. In some embodiments, the antibody or antigen binding fragment provided herein specifically binds to IL-23R competitively with an anti-IL-23R antibody comprising a VH comprising the amino acid sequence of SEQ ID NO:234, and a VL comprising the amino acid sequence of SEQ ID NO:235. In some embodiments, the antibody or antigen binding fragment provided herein specifically binds to IL-23R competitively with an anti-IL-23R antibody comprising a VH comprising the amino acid sequence of SEQ ID NO:236, and a VL comprising the amino acid sequence of SEQ ID NO:237. In some embodiments, the antibody or antigen binding fragment provided herein specifically binds to IL-23R competitively with an anti-IL-23R antibody comprising a VH comprising the amino acid sequence of SEQ ID NO:238, and a VL comprising the amino acid sequence of SEQ ID NO:239. In some embodiments, the antibody or antigen binding fragment provided herein specifically binds to IL-23R competitively with an anti-IL-23R antibody comprising a VH comprising the amino acid sequence of SEQ ID NO:240, and a VL comprising the amino acid sequence of SEQ ID NO:241. In some embodiments, the antibody or antigen binding fragment provided herein specifically binds to IL-23R competitively with an anti-IL-23R antibody comprising a VH comprising the amino acid sequence of SEQ ID NO:246, and a VL comprising the amino acid sequence of SEQ ID NO:247. In some embodiments, the antibody or antigen binding fragment provided herein specifically binds to IL-23R competitively with an anti-IL-23R antibody comprising a VH comprising the amino acid sequence of SEQ ID NO:248, and a VL comprising the amino acid sequence of SEQ ID NO:249. In some embodiments, the antibody or antigen binding fragment provided herein specifically binds to IL-23R competitively with an anti-IL-23R antibody comprising a VH comprising the amino acid sequence of SEQ ID NO:250, and a VL comprising the amino acid sequence of SEQ ID NO:251. In some embodiments, the antibody or antigen binding fragment provided herein specifically binds to IL-23R competitively with an anti-IL-23R antibody comprising a VH comprising the amino acid sequence of SEQ ID NO:252, and a VL comprising the amino acid sequence of SEQ ID NO:253. In some embodiments, the antibody or antigen binding fragment provided herein specifically binds to IL-23R competitively with an anti-IL-23R antibody comprising a VH comprising the amino acid sequence of SEQ ID NO:254, and a VL comprising the amino acid sequence of SEQ ID NO:255. In some embodiments, the antibody or antigen binding fragment provided herein specifically binds to IL-23R competitively with an anti-IL-23R antibody comprising a VH comprising the amino acid sequence of SEQ ID NO:256, and a VL comprising the amino acid sequence of SEQ ID NO:257. In some embodiments, the antibody or antigen binding fragment provided herein specifically binds to IL-23R competitively with an anti-IL-23R antibody comprising a VH comprising the amino acid sequence of SEQ ID NO:258, and a VL comprising the amino acid sequence of SEQ ID NO:259. In some embodiments, the antibody or antigen binding fragment provided herein specifically binds to IL-23R competitively with an anti-IL-23R antibody comprising a VH comprising the amino acid sequence of SEQ ID NO:260, and a VL comprising the amino acid sequence of SEQ ID NO:261. In some embodiments, the antibody or antigen binding fragment provided herein specifically binds to IL-23R competitively with an anti-IL-23R antibody comprising a VH comprising the amino acid sequence of SEQ ID NO:262, and a LCDR1, an LCDR2, and an LCDR3 as set forth in SEQ ID NO:263. In some embodiments, the antibody or antigen binding fragment provided herein specifically binds to IL-23R competitively with an anti-IL-23R antibody comprising a VH comprising the amino acid sequence of SEQ ID NO:264, and a VL comprising the amino acid sequence of SEQ ID NO:265. In some embodiments, the antibody or antigen binding fragment provided herein specifically binds to IL-23R competitively with an anti-IL-23R antibody comprising a VH comprising the amino acid sequence of SEQ ID NO:268, and a VL comprising the amino acid sequence of SEQ ID NO:269. In some embodiments, the antibody or antigen binding fragment provided herein specifically binds to IL-23R competitively with an anti-IL-23R antibody comprising a VH comprising the amino acid sequence of SEQ ID NO:786, and a VL comprising the amino acid sequence of SEQ ID NO:787. In some embodiments, the antibody or antigen binding fragment provided herein specifically binds to IL-23R competitively with an anti-IL-23R antibody comprising a VH comprising the amino acid sequence of SEQ ID NO:788, and a VL comprising the amino acid sequence of SEQ ID NO:789. In some embodiments, the antibody or antigen binding fragment provided herein specifically binds to IL-23R competitively with an anti-IL-23R antibody comprising a VH comprising the amino acid sequence of SEQ ID NO:790, and a VL comprising the amino acid sequence of SEQ ID NO:791. In some embodiments, the antibody or antigen binding fragment provided herein specifically binds to IL-23R competitively with an anti-IL-23R antibody comprising a VH comprising the amino acid sequence of SEQ ID NO:792, and a VL comprising the amino acid sequence of SEQ ID NO:793. In some embodiments, the antibody or antigen binding fragment provided herein specifically binds to IL-23R competitively with an anti-IL-23R antibody comprising a VH comprising the amino acid sequence of SEQ ID NO:798, and a VL comprising the amino acid sequence of SEQ ID NO:799. In some embodiments, the antibody or antigen binding fragment provided herein specifically binds to IL-23R competitively with an anti-IL-23R antibody comprising a VH comprising the amino acid sequence of SEQ ID NO:806, and a VL comprising the amino acid sequence of SEQ ID NO:807. In some embodiments, the antibody or antigen binding fragment provided herein specifically binds to IL-23R competitively with an anti-IL-23R antibody comprising a VH comprising the amino acid sequence of SEQ ID NO:808, and a VL comprising the amino acid sequence of SEQ ID NO:809. In some embodiments, the antibody or antigen binding fragment provided herein specifically binds to IL-23R competitively with an anti-IL-23R antibody comprising a VH comprising the amino acid sequence of SEQ ID NO:810, and a VL comprising the amino acid sequence of SEQ ID NO:811. In some embodiments, the antibody or antigen binding fragment provided herein specifically binds to IL-23R competitively with an anti-IL-23R antibody comprising a VH comprising the amino acid sequence of SEQ ID NO:812, and a VL comprising the amino acid sequence of SEQ ID NO:813. In some embodiments, the antibody or antigen binding fragment provided herein specifically binds to IL-23R competitively with an anti-IL-23R antibody comprising a VH comprising the amino acid sequence of SEQ ID NO:814, and a VL comprising the amino acid sequence of SEQ ID NO:815. In some embodiments, the antibody or antigen binding fragment provided herein specifically binds to IL-23R competitively with an anti-IL-23R antibody comprising a VH comprising the amino acid sequence of SEQ ID NO:816, and a VL comprising the amino acid sequence of SEQ ID NO:817. In some embodiments, the antibody or antigen binding fragment provided herein specifically binds to IL-23R competitively with an anti-IL-23R antibody comprising a VH comprising the amino acid sequence of SEQ ID NO:818, and a VL comprising the amino acid sequence of SEQ ID NO:819. In some embodiments, the antibody or antigen binding fragment provided herein specifically binds to IL-23R competitively with an anti-IL-23R antibody comprising a VH comprising the amino acid sequence of SEQ ID NO:820, and a VL comprising the amino acid sequence of SEQ ID NO:821. In some embodiments, the antibody or antigen binding fragment provided herein specifically binds to IL-23R competitively with an anti-IL-23R antibody comprising a VH comprising the amino acid sequence of SEQ ID NO:822, and a VL comprising the amino acid sequence of SEQ ID NO:823. In some embodiments, the antibody or antigen binding fragment provided herein specifically binds to IL-23R competitively with an anti-IL-23R antibody comprising a VH comprising the amino acid sequence of SEQ ID NO:824, and a VL comprising the amino acid sequence of SEQ ID NO:825. In some embodiments, the antibody or antigen binding fragment provided herein specifically binds to IL-23R competitively with an anti-IL-23R antibody comprising a VH comprising the amino acid sequence of SEQ ID NO:826, and a VL comprising the amino acid sequence of SEQ ID NO:827. In some embodiments, the antibody or antigen binding fragment provided herein specifically binds to IL-23R competitively with an anti-IL-23R antibody comprising a VH comprising the amino acid sequence of SEQ ID NO:828, and a VL comprising the amino acid sequence of SEQ ID NO:829. In some embodiments, the antibody or antigen binding fragment provided herein specifically binds to IL-23R competitively with an anti-IL-23R antibody comprising a VH comprising the amino acid sequence of SEQ ID NO:830, and a VL comprising the amino acid sequence of SEQ ID NO:831. In some embodiments, the antibody or antigen binding fragment provided herein specifically binds to IL-23R competitively with an anti-IL-23R antibody comprising a VH comprising the amino acid sequence of SEQ ID NO:832, and a VL comprising the amino acid sequence of SEQ ID NO:833. In some embodiments, the antibody or antigen binding fragment provided herein specifically binds to IL-23R competitively with an anti-IL-23R antibody comprising a VH comprising the amino acid sequence of SEQ ID NO:836, and a VL comprising the amino acid sequence of SEQ ID NO:837. In some embodiments, the antibody or antigen binding fragment provided herein specifically binds to IL-23R competitively with an anti-IL-23R antibody comprising a VH comprising the amino acid sequence of SEQ ID NO:838, and a VL comprising the amino acid sequence of SEQ ID NO:839. In some embodiments, the antibody or antigen binding fragment provided herein specifically binds to IL-23R competitively with an anti-IL-23R antibody comprising a VH comprising the amino acid sequence of SEQ ID NO:840, and a VL comprising the amino acid sequence of SEQ ID NO:841. In some embodiments, the antibody or antigen binding fragment provided herein specifically binds to IL-23R competitively with an anti-IL-23R antibody comprising a VH comprising the amino acid sequence of SEQ ID NO: 842, and a VL comprising the amino acid sequence of SEQ ID NO:843. In some embodiments, the antibody or antigen binding fragment provided herein specifically binds to IL-23R competitively with an anti-IL-23R antibody comprising a VH comprising the amino acid sequence of SEQ ID NO:846, and a VL comprising the amino acid sequence of SEQ ID NO:847. In some embodiments, the antibody or antigen binding fragment provided herein specifically binds to IL-23R competitively with an anti-IL-23R antibody comprising a VH comprising the amino acid sequence of SEQ ID NO:848, and a VL comprising the amino acid sequence of SEQ ID NO:849. In some embodiments, the antibody or antigen binding fragment provided herein specifically binds to IL-23R competitively with an anti-IL-23R antibody comprising a VH comprising the amino acid sequence of SEQ ID NO:850, and a VL comprising the amino acid sequence of SEQ ID NO:851. In some embodiments, the antibody or antigen binding fragment provided herein specifically binds to IL-23R competitively with an anti-IL-23R antibody comprising a VH comprising the amino acid sequence of SEQ ID NO:852, and a VL comprising the amino acid sequence of SEQ ID NO:853. In some embodiments, the antibody or antigen binding fragment provided herein specifically binds to IL-23R competitively with an anti-IL-23R antibody comprising a VH comprising the amino acid sequence of SEQ ID NO:854, and a VL comprising the amino acid sequence of SEQ ID NO:855. In some embodiments, the antibody or antigen binding fragment provided herein specifically binds to IL-23R competitively with an anti-IL-23R antibody comprising a VH comprising the amino acid sequence of SEQ ID NO:856, and a VL comprising the amino acid sequence of SEQ ID NO:857. In some embodiments, the antibody or antigen binding fragment provided herein specifically binds to IL-23R competitively with an anti-IL-23R antibody comprising a VH comprising the amino acid sequence of SEQ ID NO:858, and a VL comprising the amino acid sequence of SEQ ID NO:859. In some embodiments, the antibody or antigen binding fragment provided herein specifically binds to IL-23R competitively with an anti-IL-23R antibody comprising a VH comprising the amino acid sequence of SEQ ID NO:860, and a VL comprising the amino acid sequence of SEQ ID NO:861. In some embodiments, the antibody or antigen binding fragment provided herein specifically binds to IL-23R competitively with an anti-IL-23R antibody comprising a VH comprising the amino acid sequence of SEQ ID NO:862, and a VL comprising the amino acid sequence of SEQ ID NO:863. In some embodiments, the antibody or antigen binding fragment provided herein specifically binds to IL-23R competitively with an anti-IL-23R antibody comprising a VH comprising the amino acid sequence of SEQ ID NO:864, and a VL comprising the amino acid sequence of SEQ ID NO:865. In some embodiments, the antibody or antigen binding fragment provided herein specifically binds to IL-23R competitively with an anti-IL-23R antibody comprising a VH comprising the amino acid sequence of SEQ ID NO:866, and a VL comprising the amino acid sequence of SEQ ID NO:867. In some embodiments, the antibody or antigen binding fragment provided herein specifically binds to IL-23R competitively with an anti-IL-23R antibody comprising a VH comprising the amino acid sequence of SEQ ID NO:868, and a VL comprising the amino acid sequence of SEQ ID NO:869. In some embodiments, the antibody or antigen binding fragment provided herein specifically binds to IL-23R competitively with an anti-IL-23R antibody comprising a VH comprising the amino acid sequence of SEQ ID NO:870, and a VL comprising the amino acid sequence of SEQ ID NO:871. In some embodiments, the antibody or antigen binding fragment provided herein specifically binds to IL-23R competitively with an anti-IL-23R antibody comprising a VH comprising the amino acid sequence of SEQ ID NO:872, and a VL comprising the amino acid sequence of SEQ ID NO:873. In some embodiments, the antibody or antigen binding fragment provided herein specifically binds to IL-23R competitively with an anti-IL-23R antibody comprising a VH comprising the amino acid sequence of SEQ ID NO:874, and a VL comprising the amino acid sequence of SEQ ID NO:875. In some embodiments, the antibody or antigen binding fragment provided herein specifically binds to IL-23R competitively with an anti-IL-23R antibody comprising a VH comprising the amino acid sequence of SEQ ID NO:876, and a VL comprising the amino acid sequence of SEQ ID NO:877. In some embodiments, the antibody or antigen binding fragment provided herein specifically binds to IL-23R competitively with an anti-IL-23R antibody comprising a VH comprising the amino acid sequence of SEQ ID NO:878, and a VL comprising the amino acid sequence of SEQ ID NO:879. In some embodiments, the antibody or antigen binding fragment provided herein specifically binds to IL-23R competitively with an anti-IL-23R antibody comprising a VH comprising the amino acid sequence of SEQ ID NO:880, and a VL comprising the amino acid sequence of SEQ ID NO:881. In some embodiments, the antibody or antigen binding fragment provided herein specifically binds to IL-23R competitively with an anti-IL-23R antibody comprising a VH comprising the amino acid sequence of SEQ ID NO:882, and a VL comprising the amino acid sequence of SEQ ID NO:883. In some embodiments, the antibody or antigen binding fragment provided herein specifically binds to IL-23R competitively with an anti-IL-23R antibody comprising a VH comprising the amino acid sequence of SEQ ID NO:884, and a VL comprising the amino acid sequence of SEQ ID NO:885. In some embodiments, the antibody or antigen binding fragment provided herein specifically binds to IL-23R competitively with an anti-IL-23R antibody comprising a VH comprising the amino acid sequence of SEQ ID NO:886, and a VL comprising the amino acid sequence of SEQ ID NO:887. In some embodiments, the antibody or antigen binding fragment provided herein specifically binds to IL-23R competitively with an anti-IL-23R antibody comprising a VH comprising the amino acid sequence of SEQ ID NO:888, and a VL comprising the amino acid sequence of SEQ ID NO:889. In some embodiments, the antibody or antigen binding fragment provided herein specifically binds to IL-23R competitively with an anti-IL-23R antibody comprising a VH comprising the amino acid sequence of SEQ ID NO:890, and a VL comprising the amino acid sequence of SEQ ID NO:891.

In some embodiments, the antibody comprises a heavy chain comprising the amino acid sequence of SEQ ID NO:4 and a light chain comprising the amino acid sequence of SEQ ID NO:5. In some embodiments, the antibody comprises a heavy chain comprising the amino acid sequence of SEQ ID NO:8 and a light chain comprising the amino acid sequence of SEQ ID NO:9. In some embodiments, the antibody comprises a heavy chain comprising the amino acid sequence of SEQ ID NO:10 and a light chain comprising the amino acid sequence of SEQ ID NO:11. In some embodiments, the antibody comprises a heavy chain comprising the amino acid sequence of SEQ ID NO:14 and a light chain comprising the amino acid sequence of SEQ ID NO:15. In some embodiments, the antibody comprises a heavy chain comprising the amino acid sequence of SEQ ID NO:16 and a light chain comprising the amino acid sequence of SEQ ID NO:17. In some embodiments, the antibody comprises a heavy chain comprising the amino acid sequence of SEQ ID NO:18 and a light chain comprising the amino acid sequence of SEQ ID NO:19. In some embodiments, the antibody comprises a heavy chain comprising the amino acid sequence of SEQ ID NO:20 and a light chain comprising the amino acid sequence of SEQ ID NO:21. In some embodiments, the antibody comprises a heavy chain comprising the amino acid sequence of SEQ ID NO:22 and a light chain comprising the amino acid sequence of SEQ ID NO:23. In some embodiments, the antibody comprises a heavy chain comprising the amino acid sequence of SEQ ID NO:24 and a light chain comprising the amino acid sequence of SEQ ID NO:25. In some embodiments, the antibody comprises a heavy chain comprising the amino acid sequence of SEQ ID NO:26 and a light chain comprising the amino acid sequence of SEQ ID NO:27. In some embodiments, the antibody comprises a heavy chain comprising the amino acid sequence of SEQ ID NO:28 and a light chain comprising the amino acid sequence of SEQ ID NO:29. In some embodiments, the antibody comprises a heavy chain comprising the amino acid sequence of SEQ ID NO:30 and a light chain comprising the amino acid sequence of SEQ ID NO:31. In some embodiments, the antibody comprises a heavy chain comprising the amino acid sequence of SEQ ID NO:32 and a light chain comprising the amino acid sequence of SEQ ID NO:33. In some embodiments, the antibody comprises a heavy chain comprising the amino acid sequence of SEQ ID NO:34 and a light chain comprising the amino acid sequence of SEQ ID NO:35. In some embodiments, the antibody comprises a heavy chain comprising the amino acid sequence of SEQ ID NO:36 and a light chain comprising the amino acid sequence of SEQ ID NO:37. In some embodiments, the antibody comprises a heavy chain comprising the amino acid sequence of SEQ ID NO:38 and a light chain comprising the amino acid sequence of SEQ ID NO:39. In some embodiments, the antibody comprises a heavy chain comprising the amino acid sequence of SEQ ID NO:40 and a light chain comprising the amino acid sequence of SEQ ID NO:41. In some embodiments, the antibody comprises a heavy chain comprising the amino acid sequence of SEQ ID NO:42 and a light chain comprising the amino acid sequence of SEQ ID NO:43. In some embodiments, the antibody comprises a heavy chain comprising the amino acid sequence of SEQ ID NO:44 and a light chain comprising the amino acid sequence of SEQ ID NO:45. In some embodiments, the antibody comprises a heavy chain comprising the amino acid sequence of SEQ ID NO:46 and a light chain comprising the amino acid sequence of SEQ ID NO:47. In some embodiments, the antibody comprises a heavy chain comprising the amino acid sequence of SEQ ID NO:48 and a light chain comprising the amino acid sequence of SEQ ID NO:49. In some embodiments, the antibody comprises a heavy chain comprising the amino acid sequence of SEQ ID NO:52 and a light chain comprising the amino acid sequence of SEQ ID NO:53. In some embodiments, the antibody comprises a heavy chain comprising the amino acid sequence of SEQ ID NO:54 and a light chain comprising the amino acid sequence of SEQ ID NO:55. In some embodiments, the antibody comprises a heavy chain comprising the amino acid sequence of SEQ ID NO:56 and a light chain comprising the amino acid sequence of SEQ ID NO:57. In some embodiments, the antibody comprises a heavy chain comprising the amino acid sequence of SEQ ID NO:58 and a light chain comprising the amino acid sequence of SEQ ID NO:59. In some embodiments, the antibody comprises a heavy chain comprising the amino acid sequence of SEQ ID NO:60 and a light chain comprising the amino acid sequence of SEQ ID NO:61. In some embodiments, the antibody comprises a heavy chain comprising the amino acid sequence of SEQ ID NO:62 and a light chain comprising the amino acid sequence of SEQ ID NO:63. In some embodiments, the antibody comprises a heavy chain comprising the amino acid sequence of SEQ ID NO:64 and a light chain comprising the amino acid sequence of SEQ ID NO:65. In some embodiments, the antibody comprises a heavy chain comprising the amino acid sequence of SEQ ID NO:66 and a light chain comprising the amino acid sequence of SEQ ID NO:67. In some embodiments, the antibody comprises a heavy chain comprising the amino acid sequence of SEQ ID NO:68 and a light chain comprising the amino acid sequence of SEQ ID NO:69. In some embodiments, the antibody comprises a heavy chain comprising the amino acid sequence of SEQ ID NO:70 and a light chain comprising the amino acid sequence of SEQ ID NO:71. In some embodiments, the antibody comprises a heavy chain comprising the amino acid sequence of SEQ ID NO:72 and a light chain comprising the amino acid sequence of SEQ ID NO:73. In some embodiments, the antibody comprises a heavy chain comprising the amino acid sequence of SEQ ID NO:74 and a light chain comprising the amino acid sequence of SEQ ID NO:75. In some embodiments, the antibody comprises a heavy chain comprising the amino acid sequence of SEQ ID NO:76 and a light chain comprising the amino acid sequence of SEQ ID NO:77. In some embodiments, the antibody comprises a heavy chain comprising the amino acid sequence of SEQ ID NO:78 and a light chain comprising the amino acid sequence of SEQ ID NO:79. In some embodiments, the antibody comprises a heavy chain comprising the amino acid sequence of SEQ ID NO:80 and a light chain comprising the amino acid sequence of SEQ ID NO:81. In some embodiments, the antibody comprises a heavy chain comprising the amino acid sequence of SEQ ID NO:82 and a light chain comprising the amino acid sequence of SEQ ID NO:83. In some embodiments, the antibody comprises a heavy chain comprising the amino acid sequence of SEQ ID NO:84 and a light chain comprising the amino acid sequence of SEQ ID NO:85. In some embodiments, the antibody comprises a heavy chain comprising the amino acid sequence of SEQ ID NO:86 and a light chain comprising the amino acid sequence of SEQ ID NO:87. In some embodiments, the antibody comprises a heavy chain comprising the amino acid sequence of SEQ ID NO:88 and a light chain comprising the amino acid sequence of SEQ ID NO:89. In some embodiments, the antibody comprises a heavy chain comprising the amino acid sequence of SEQ ID NO:92 and a light chain comprising the amino acid sequence of SEQ ID NO:93. In some embodiments, the antibody comprises a heavy chain comprising the amino acid sequence of SEQ ID NO:94 and a light chain comprising the amino acid sequence of SEQ ID NO:95. In some embodiments, the antibody comprises a heavy chain comprising the amino acid sequence of SEQ ID NO:96 and a light chain comprising the amino acid sequence of SEQ ID NO:97. In some embodiments, the antibody comprises a heavy chain comprising the amino acid sequence of SEQ ID NO:98 and a light chain comprising the amino acid sequence of SEQ ID NO:99. In some embodiments, the antibody comprises a heavy chain comprising the amino acid sequence of SEQ ID NO:100 and a light chain comprising the amino acid sequence of SEQ ID NO:101. In some embodiments, the antibody comprises a heavy chain comprising the amino acid sequence of SEQ ID NO:102 and a light chain comprising the amino acid sequence of SEQ ID NO:103. In some embodiments, the antibody comprises a heavy chain comprising the amino acid sequence of SEQ ID NO:104 and a light chain comprising the amino acid sequence of SEQ ID NO:105. In some embodiments, the antibody comprises a heavy chain comprising the amino acid sequence of SEQ ID NO:106 and a light chain comprising the amino acid sequence of SEQ ID NO:107. In some embodiments, the antibody comprises a heavy chain comprising the amino acid sequence of SEQ ID NO:112 and a light chain comprising the amino acid sequence of SEQ ID NO:113. In some embodiments, the antibody comprises a heavy chain comprising the amino acid sequence of SEQ ID NO:114 and a light chain comprising the amino acid sequence of SEQ ID NO:115. In some embodiments, the antibody comprises a heavy chain comprising the amino acid sequence of SEQ ID NO:116 and a light chain comprising the amino acid sequence of SEQ ID NO:117. In some embodiments, the antibody comprises a heavy chain comprising the amino acid sequence of SEQ ID NO:118 and a light chain comprising the amino acid sequence of SEQ ID NO:119. In some embodiments, the antibody comprises a heavy chain comprising the amino acid sequence of SEQ ID NO:120 and a light chain comprising the amino acid sequence of SEQ ID NO:121. In some embodiments, the antibody comprises a heavy chain comprising the amino acid sequence of SEQ ID NO:122 and a light chain comprising the amino acid sequence of SEQ ID NO:123. In some embodiments, the antibody comprises a heavy chain comprising the amino acid sequence of SEQ ID NO:124 and a light chain comprising the amino acid sequence of SEQ ID NO:125. In some embodiments, the antibody comprises a heavy chain comprising the amino acid sequence of SEQ ID NO:126 and a light chain comprising the amino acid sequence of SEQ ID NO:127. In some embodiments, the antibody comprises a heavy chain comprising the amino acid sequence of SEQ ID NO:128 and a light chain comprising the amino acid sequence of SEQ ID NO:129. In some embodiments, the antibody comprises a heavy chain comprising the amino acid sequence of SEQ ID NO:130 and a light chain comprising the amino acid sequence of SEQ ID NO:131. In some embodiments, the antibody comprises a heavy chain comprising the amino acid sequence of SEQ ID NO:134 and a light chain comprising the amino acid sequence of SEQ ID NO:135. In some embodiments, the antibody comprises a heavy chain comprising the amino acid sequence of SEQ ID NO:672 and a light chain comprising the amino acid sequence of SEQ ID NO:673. In some embodiments, the antibody comprises a heavy chain comprising the amino acid sequence of SEQ ID NO:674 and a light chain comprising the amino acid sequence of SEQ ID NO:675. In some embodiments, the antibody comprises a heavy chain comprising the amino acid sequence of SEQ ID NO:676 and a light chain comprising the amino acid sequence of SEQ ID NO:677. In some embodiments, the antibody comprises a heavy chain comprising the amino acid sequence of SEQ ID NO:678 and a light chain comprising the amino acid sequence of SEQ ID NO:679. In some embodiments, the antibody comprises a heavy chain comprising the amino acid sequence of SEQ ID NO:684 and a light chain comprising the amino acid sequence of SEQ ID NO:685. In some embodiments, the antibody comprises a heavy chain comprising the amino acid sequence of SEQ ID NO:692 and a light chain comprising the amino acid sequence of SEQ ID NO:693. In some embodiments, the antibody comprises a heavy chain comprising the amino acid sequence of SEQ ID NO:694 and a light chain comprising the amino acid sequence of SEQ ID NO:695. In some embodiments, the antibody comprises a heavy chain comprising the amino acid sequence of SEQ ID NO:696 and a light chain comprising the amino acid sequence of SEQ ID NO:697. In some embodiments, the antibody comprises a heavy chain comprising the amino acid sequence of SEQ ID NO:698 and a light chain comprising the amino acid sequence of SEQ ID NO:699. In some embodiments, the antibody comprises a heavy chain comprising the amino acid sequence of SEQ ID NO:700 and a light chain comprising the amino acid sequence of SEQ ID NO:701. In some embodiments, the antibody comprises a heavy chain comprising the amino acid sequence of SEQ ID NO:702 and a light chain comprising the amino acid sequence of SEQ ID NO:703. In some embodiments, the antibody comprises a heavy chain comprising the amino acid sequence of SEQ ID NO:704 and a light chain comprising the amino acid sequence of SEQ ID NO:705. In some embodiments, the antibody comprises a heavy chain comprising the amino acid sequence of SEQ ID NO:706 and a light chain comprising the amino acid sequence of SEQ ID NO:707. In some embodiments, the antibody comprises a heavy chain comprising the amino acid sequence of SEQ ID NO:708 and a light chain comprising the amino acid sequence of SEQ ID NO:709. In some embodiments, the antibody comprises a heavy chain comprising the amino acid sequence of SEQ ID NO:710 and a light chain comprising the amino acid sequence of SEQ ID NO:711. In some embodiments, the antibody comprises a heavy chain comprising the amino acid sequence of SEQ ID NO:712 and a light chain comprising the amino acid sequence of SEQ ID NO:713. In some embodiments, the antibody comprises a heavy chain comprising the amino acid sequence of SEQ ID NO:714 and a light chain comprising the amino acid sequence of SEQ ID NO:715. In some embodiments, the antibody comprises a heavy chain comprising the amino acid sequence of SEQ ID NO:716 and a light chain comprising the amino acid sequence of SEQ ID NO:717. In some embodiments, the antibody comprises a heavy chain comprising the amino acid sequence of SEQ ID NO:718 and a light chain comprising the amino acid sequence of SEQ ID NO:719. In some embodiments, the antibody comprises a heavy chain comprising the amino acid sequence of SEQ ID NO:722 and a light chain comprising the amino acid sequence of SEQ ID NO:723. In some embodiments, the antibody comprises a heavy chain comprising the amino acid sequence of SEQ ID NO:724 and a light chain comprising the amino acid sequence of SEQ ID NO:725. In some embodiments, the antibody comprises a heavy chain comprising the amino acid sequence of SEQ ID NO:726 and a light chain comprising the amino acid sequence of SEQ ID NO:727. In some embodiments, the antibody comprises a heavy chain comprising the amino acid sequence of SEQ ID NO:728 and a light chain comprising the amino acid sequence of SEQ ID NO:729. In some embodiments, the antibody comprises a heavy chain comprising the amino acid sequence of SEQ ID NO:732 and a light chain comprising the amino acid sequence of SEQ ID NO:733. In some embodiments, the antibody comprises a heavy chain comprising the amino acid sequence of SEQ ID NO:734 and a light chain comprising the amino acid sequence of SEQ ID NO:735. In some embodiments, the antibody comprises a heavy chain comprising the amino acid sequence of SEQ ID NO:736 and a light chain comprising the amino acid sequence of SEQ ID NO:737. In some embodiments, the antibody comprises a heavy chain comprising the amino acid sequence of SEQ ID NO:738 and a light chain comprising the amino acid sequence of SEQ ID NO:739. In some embodiments, the antibody comprises a heavy chain comprising the amino acid sequence of SEQ ID NO:740 and a light chain comprising the amino acid sequence of SEQ ID NO:741. In some embodiments, the antibody comprises a heavy chain comprising the amino acid sequence of SEQ ID NO:742 and a light chain comprising the amino acid sequence of SEQ ID NO:743. In some embodiments, the antibody comprises a heavy chain comprising the amino acid sequence of SEQ ID NO:744 and a light chain comprising the amino acid sequence of SEQ ID NO:745. In some embodiments, the antibody comprises a heavy chain comprising the amino acid sequence of SEQ ID NO:746 and a light chain comprising the amino acid sequence of SEQ ID NO:747. In some embodiments, the antibody comprises a heavy chain comprising the amino acid sequence of SEQ ID NO:748 and a light chain comprising the amino acid sequence of SEQ ID NO:749. In some embodiments, the antibody comprises a heavy chain comprising the amino acid sequence of SEQ ID NO:750 and a light chain comprising the amino acid sequence of SEQ ID NO:751. In some embodiments, the antibody comprises a heavy chain comprising the amino acid sequence of SEQ ID NO:752 and a light chain comprising the amino acid sequence of SEQ ID NO:753. In some embodiments, the antibody comprises a heavy chain comprising the amino acid sequence of SEQ ID NO:754 and a light chain comprising the amino acid sequence of SEQ ID NO:755. In some embodiments, the antibody comprises a heavy chain comprising the amino acid sequence of SEQ ID NO:756 and a light chain comprising the amino acid sequence of SEQ ID NO:757. In some embodiments, the antibody comprises a heavy chain comprising the amino acid sequence of SEQ ID NO:758 and a light chain comprising the amino acid sequence of SEQ ID NO:759. In some embodiments, the antibody comprises a heavy chain comprising the amino acid sequence of SEQ ID NO:760 and a light chain comprising the amino acid sequence of SEQ ID NO:761. In some embodiments, the antibody comprises a heavy chain comprising the amino acid sequence of SEQ ID NO:762 and a light chain comprising the amino acid sequence of SEQ ID NO:763. In some embodiments, the antibody comprises a heavy chain comprising the amino acid sequence of SEQ ID NO:764 and a light chain comprising the amino acid sequence of SEQ ID NO:765. In some embodiments, the antibody comprises a heavy chain comprising the amino acid sequence of SEQ ID NO:766 and a light chain comprising the amino acid sequence of SEQ ID NO:767. In some embodiments, the antibody comprises a heavy chain comprising the amino acid sequence of SEQ ID NO:768 and a light chain comprising the amino acid sequence of SEQ ID NO:769. In some embodiments, the antibody comprises a heavy chain comprising the amino acid sequence of SEQ ID NO:770 and a light chain comprising the amino acid sequence of SEQ ID NO:771. In some embodiments, the antibody comprises a heavy chain comprising the amino acid sequence of SEQ ID NO:772 and a light chain comprising the amino acid sequence of SEQ ID NO:773. In some embodiments, the antibody comprises a heavy chain comprising the amino acid sequence of SEQ ID NO:774 and a light chain comprising the amino acid sequence of SEQ ID NO:775. In some embodiments, the antibody comprises a heavy chain comprising the amino acid sequence of SEQ ID NO:776 and a light chain comprising the amino acid sequence of SEQ ID NO:777.

In some embodiments, provided herein is an IL-23R binding protein comprising any one of the anti-IL-23R antibodies described above. In some embodiments, the IL-23R binding protein is an antibody comprising two heavy chains and two light chains. In some embodiments, the IL-23R binding protein is an antibody comprising two heavy chains comprising a same VH region and two light chains comprising a same VL region.

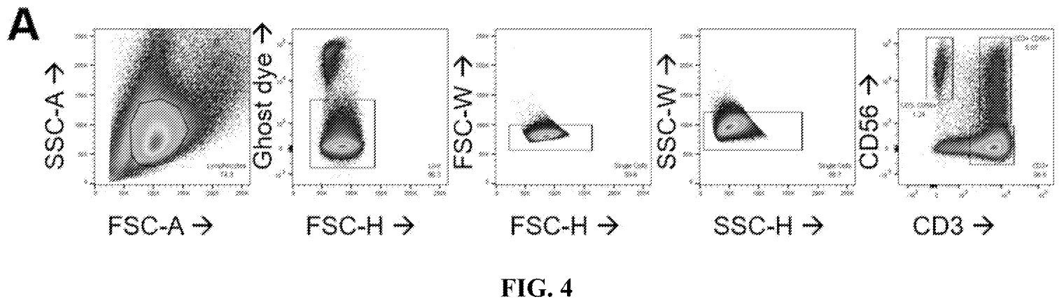

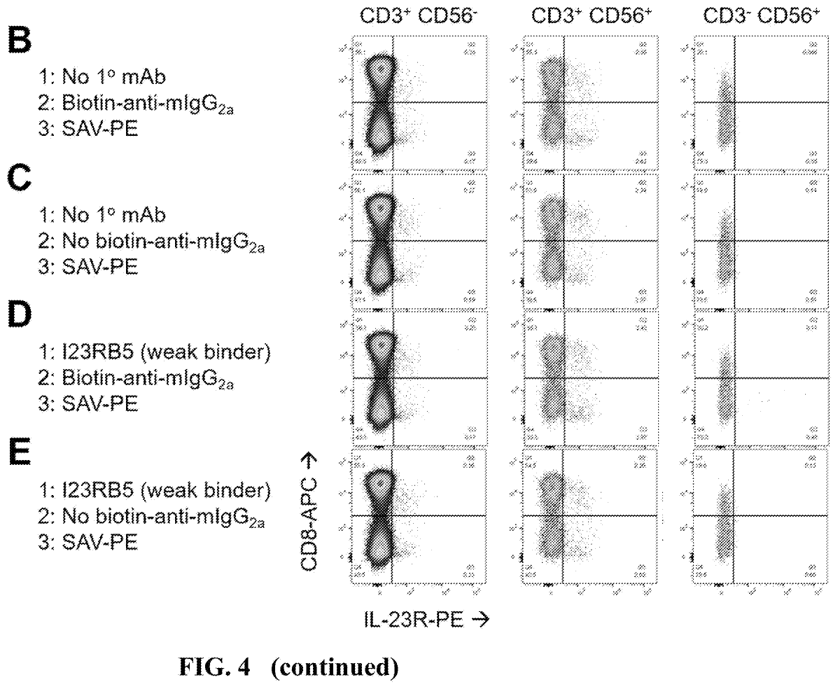

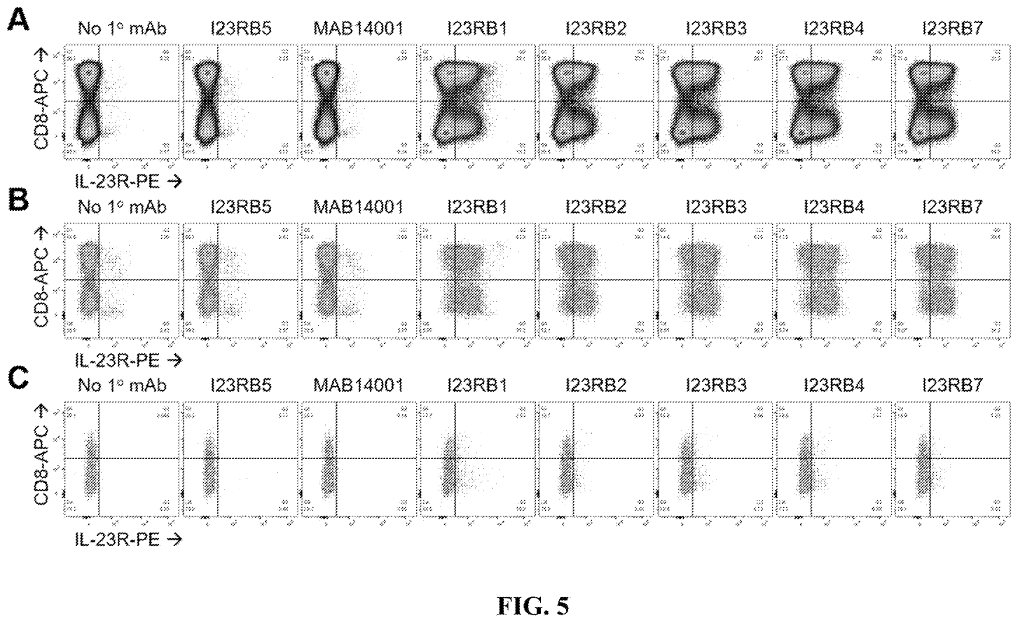

In a particular embodiment, antibodies or antigen binding fragments thereof are provided which were isolated in accordance with the procedures and processes provided herein and which upon binding analysis do not demonstrate particular or significant affinity or binding to IL-23R, or at least do not demonstrate binding or relevant affinity as tested herein, or do not demonstrate binding to the I23R ECD. These can be designated as non binders or as weak binders. These antibodies provide utility as matched or related controls in any of various assays or methods, including when used in combination with the demonstrated IL23R binding antibodies herein. For instance antibody I23RB5 does not show binding to IL-23R but is utilized in experiments provided herein, for example as set out in FIGS. 4 and 5 as a non-binder or weak binder, in such instance for example as a control antibody. The antibodies have unique and novel sequences, including VH, HC and H chain CDR sequences, and in many or most instances also have unique VL, LC and light chain CDR sequences. The following antibodies can be denoted as novel antibodies, without significant or recognized binding to IL23R: I23RB5, I23RB6, I23RB8, I23RB9, I23RB10, I23RB 80, I23RB101, I23RB104, I23RB149, I23RB189, I23RB273, I23RB278, I23RB328, I23RB329, I23RB330, and I23RB332.

These antibodies correspond to antibodies having sequences as indicated below:

In some embodiments, the antibody or antigen binding fragment provided herein comprises an HCDR1, an HCDR2, and an HCDR3 as set forth in SEQ ID NO:136, and an LCDR1, an LCDR2, and an LCDR3 as set forth in SEQ ID NO:137. In some embodiments, the antibody or antigen binding fragment comprises an HCDR1, an HCDR2, and an HCDR3 as set forth in SEQ ID NO:140, and an LCDR1, an LCDR2, and an LCDR3 as set forth in SEQ ID NO:141. In some embodiments, the antibody or antigen binding fragment comprises an HCDR1, an HCDR2, and an HCDR3 as set forth in SEQ ID NO:146, and an LCDR1, an LCDR2, and an LCDR3 as set forth in SEQ ID NO:147. In some embodiments, the antibody or antigen binding fragment comprises an HCDR1, an HCDR2, and an HCDR3 as set forth in SEQ ID NO:184, and an LCDR1, an LCDR2, and an LCDR3 as set forth in SEQ ID NO:185. In some embodiments, the antibody or antigen binding fragment comprises an HCDR1, an HCDR2, and an HCDR3 as set forth in SEQ ID NO:244, and an LCDR1, an LCDR2, and an LCDR3 as set forth in SEQ ID NO:245. In some embodiments, the antibody or antigen binding fragment comprises an HCDR1, an HCDR2, and an HCDR3 as set forth in SEQ ID NO:794, and an LCDR1, an LCDR2, and an LCDR3 as set forth in SEQ ID NO:795. In some embodiments, the antibody or antigen binding fragment comprises an HCDR1, an HCDR2, and an HCDR3 as set forth in SEQ ID NO:796, and an LCDR1, an LCDR2, and an LCDR3 as set forth in SEQ ID NO:797. In some embodiments, the antibody or antigen binding fragment comprises an HCDR1, an HCDR2, and an HCDR3 as set forth in SEQ ID NO:800, and an LCDR1, an LCDR2, and an LCDR3 as set forth in SEQ ID NO:801. In some embodiments, the antibody or antigen binding fragment comprises an HCDR1, an HCDR2, and an HCDR3 as set forth in SEQ ID NO:802, and an LCDR1, an LCDR2, and an LCDR3 as set forth in SEQ ID NO:803. In some embodiments, the antibody or antigen binding fragment comprises an HCDR1, an HCDR2, and an HCDR3 as set forth in SEQ ID NO:804, and an LCDR1, an LCDR2, and an LCDR3 as set forth in SEQ ID NO:805. In some embodiments, the antibody or antigen binding fragment comprises an HCDR1, an HCDR2, and an HCDR3 as set forth in SEQ ID NO:834, and an LCDR1, an LCDR2, and an LCDR3 as set forth in SEQ ID NO:835. In some embodiments, the antibody or antigen binding fragment comprises an HCDR1, an HCDR2, and an HCDR3 as set forth in SEQ ID NO:844, and an LCDR1, an LCDR2, and an LCDR3 as set forth in SEQ ID NO:845. In some embodiments, the antibody or antigen binding fragment comprises an HCDR1, an HCDR2, and an HCDR3 as set forth in SEQ ID NO:892, and an LCDR1, an LCDR2, and an LCDR3 as set forth in SEQ ID NO:893. In some embodiments, the antibody or antigen binding fragment comprises an HCDR1, an HCDR2, and an HCDR3 as set forth in SEQ ID NO:894, and an LCDR1, an LCDR2, and an LCDR3 as set forth in SEQ ID NO:895. In some embodiments, the antibody or antigen binding fragment comprises an HCDR1, an HCDR2, and an HCDR3 as set forth in SEQ ID NO:896, and an LCDR1, an LCDR2, and an LCDR3 as set forth in SEQ ID NO:897. In some embodiments, the antibody or antigen binding fragment comprises an HCDR1, an HCDR2, and an HCDR3 as set forth in SEQ ID NO:898 and an LCDR1, an LCDR2, and an LCDR3 as set forth in SEQ ID NO:899. In other embodiments, an antibody is provided comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 270, 282, 300, 414, 594, 924, 930, 942, 948, 954, 1044, 1074, 1218, 1224, 1230, and 1236; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 271, 283, 301, 415, 595, 925, 931, 943, 949, 955, 1045, 1075, 1219, 1225, 1231, and 1237, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 272, 284, 302, 416, 596, 926, 932, 944, 950, 956, 1046, 1076, 1220, 1226, 1232, and 1238; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 273, 285, 303, 417, 597, 927, 933, 945, 951, 957, 1047, 1077, 1221, 1227, 1233, and 1239; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 274, 286, 304, 418, 598, 928, 934, 946, 952, 958, 1048, 1078, 1222, 1228, 1234, and 1240; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to any of SEQ ID NOs: 275, 287, 305, 419, 599, 929, 935, 947, 953, 959, 1049, 1079, 1223, 1229, 1235, and 1241. In some specific embodiments, an antibody is provided comprising: (i) a VH comprising an HCDR1, an HCDR2, and an HCDR3 having an amino acid sequence of SEQ ID NOs:270, 271, and 272, respectively, and (ii) an VL comprising an LCDR1, an LCDR2, and an LCDR3 having an amino acid sequence of SEQ ID NOs:273, 274, and 275, respectively. In some specific embodiments, provided herein is an antibody comprising: (i) a VH comprising an HCDR1, an HCDR2, and an HCDR3 having an amino acid sequence of SEQ ID NOs:282, 283, and 284, respectively, and (ii) an VL comprising an LCDR1, an LCDR2, and an LCDR3 having an amino acid sequence of SEQ ID NOs:285, 286, and 287, respectively. In some specific embodiments, provided herein is an antibody comprising: (i) a VH comprising an HCDR1, an HCDR2, and an HCDR3 having an amino acid sequence of SEQ ID NOs:300, 301, and 302, respectively, and (ii) an VL comprising an LCDR1, an LCDR2, and an LCDR3 having an amino acid sequence of SEQ ID NOs:303, 304, and 305, respectively. In some specific embodiments, provided herein is an antibody comprising: (i) a VH comprising an HCDR1, an HCDR2, and an HCDR3 having an amino acid sequence of SEQ ID NOs:414, 415, and 416, respectively, and (ii) an VL comprising an LCDR1, an LCDR2, and an LCDR3 having an amino acid sequence of SEQ ID NOs:417, 418, and 419, respectively. In some specific embodiments, provided herein is an antibody comprising: (i) a VH comprising an HCDR1, an HCDR2, and an HCDR3 having an amino acid sequence of SEQ ID NOs:594, 595, and 596, respectively, and (ii) an VL comprising an LCDR1, an LCDR2, and an LCDR3 having an amino acid sequence of SEQ ID NOs:597, 598, and 599, respectively. In some specific embodiments, provided herein is an antibody comprising: (i) a VH comprising an HCDR1, an HCDR2, and an HCDR3 having an amino acid sequence of SEQ ID NOs:924, 925, and 926, respectively, and (ii) an VL comprising an LCDR1, an LCDR2, and an LCDR3 having an amino acid sequence of SEQ ID NOs:927, 928, and 929, respectively. In some specific embodiments, provided herein is an antibody comprising: (i) a VH comprising an HCDR1, an HCDR2, and an HCDR3 having an amino acid sequence of SEQ ID NOs:930, 931, and 932, respectively, and (ii) an VL comprising an LCDR1, an LCDR2, and an LCDR3 having an amino acid sequence of SEQ ID NOs:933, 934, and 935, respectively. In some specific embodiments, provided herein is an antibody comprising: (i) a VH comprising an HCDR1, an HCDR2, and an HCDR3 having an amino acid sequence of SEQ ID NOs:942, 943, and 944, respectively, and (ii) an VL comprising an LCDR1, an LCDR2, and an LCDR3 having an amino acid sequence of SEQ ID NOs:945, 946, and 947, respectively. In some specific embodiments, provided herein is an antibody comprising: (i) a VH comprising an HCDR1, an HCDR2, and an HCDR3 having an amino acid sequence of SEQ ID NOs:948, 949, and 950, respectively, and (ii) an VL comprising an LCDR1, an LCDR2, and an LCDR3 having an amino acid sequence of SEQ ID NOs:951, 952, and 953, respectively. In some specific embodiments, provided herein is an antibody comprising: (i) a VH comprising an HCDR1, an HCDR2, and an HCDR3 having an amino acid sequence of SEQ ID NOs:954, 955, and 956, respectively, and (ii) an VL comprising an LCDR1, an LCDR2, and an LCDR3 having an amino acid sequence of SEQ ID NOs:957, 958, and 959, respectively. In some specific embodiments, provided herein is an antibody comprising: (i) a VH comprising an HCDR1, an HCDR2, and an HCDR3 having an amino acid sequence of SEQ ID NOs:1044, 1045, and 1046, respectively, and (ii) an VL comprising an LCDR1, an LCDR2, and an LCDR3 having an amino acid sequence of SEQ ID NOs:1047, 1048, and 1049, respectively. In some specific embodiments, provided herein is an antibody comprising: (i) a VH comprising an HCDR1, an HCDR2, and an HCDR3 having an amino acid sequence of SEQ ID NOs:1074, 1075, and 1076, respectively, and (ii) an VL comprising an LCDR1, an LCDR2, and an LCDR3 having an amino acid sequence of SEQ ID NOs:1077, 1078, and 1079, respectively. In some specific embodiments, provided herein is an antibody comprising: (i) a VH comprising an HCDR1, an HCDR2, and an HCDR3 having an amino acid sequence of SEQ ID NOs:1218, 1219, and 1220, respectively, and (ii) an VL comprising an LCDR1, an LCDR2, and an LCDR3 having an amino acid sequence of SEQ ID NOs:1221, 1222, and 1223, respectively. In some specific embodiments, provided herein is an antibody comprising: (i) a VH comprising an HCDR1, an HCDR2, and an HCDR3 having an amino acid sequence of SEQ ID NOs:1224, 1225, and 1226, respectively, and (ii) an VL comprising an LCDR1, an LCDR2, and an LCDR3 having an amino acid sequence of SEQ ID NOs:1227, 1228, and 1229, respectively. In some specific embodiments, provided herein is an antibody comprising: (i) a VH comprising an HCDR1, an HCDR2, and an HCDR3 having an amino acid sequence of SEQ ID NOs:1230, 1231, and 1232, respectively, and (ii) an VL comprising an LCDR1, an LCDR2, and an LCDR3 having an amino acid sequence of SEQ ID NOs:1233, 1234, and 1235, respectively. In some specific embodiments, provided herein is an antibody comprising: (i) a VH comprising an HCDR1, an HCDR2, and an HCDR3 having an amino acid sequence of SEQ ID NOs:1236, 1237, and 1238, respectively, and (ii) an VL comprising an LCDR1, an LCDR2, and an LCDR3 having an amino acid sequence of SEQ ID NOs:1239, 1240, and 1241, respectively. In some embodiments, the antibody or antigen binding fragment provided herein comprises a VH comprising the amino acid sequence of SEQ ID NO:136, and a VL comprising the amino acid sequence of SEQ ID NO:137. In some embodiments, the antibody or antigen binding fragment provided herein comprises a VH comprising the amino acid sequence of SEQ ID NO:140, and a VL comprising the amino acid sequence of SEQ ID NO:141. In some embodiments, the antibody or antigen binding fragment provided herein comprises a VH comprising the amino acid sequence of SEQ ID NO:146, and a VL comprising the amino acid sequence of SEQ ID NO: 147. In some embodiments, the antibody or antigen binding fragment provided herein comprises a VH comprising the amino acid sequence of SEQ ID NO:184, and a VL comprising the amino acid sequence of SEQ ID NO: 185. In some embodiments, the antibody or antigen binding fragment provided herein comprises a VH comprising the amino acid sequence of SEQ ID NO:244, and a VL comprising the amino acid sequence of SEQ ID NO:245. In some embodiments, the antibody or antigen binding fragment provided herein comprises a VH comprising the amino acid sequence of SEQ ID NO:794, and a VL comprising the amino acid sequence of SEQ ID NO:795. In some embodiments, the antibody or antigen binding fragment provided herein comprises a VH comprising the amino acid sequence of SEQ ID NO:796, and a VL comprising the amino acid sequence of SEQ ID NO:797. In some embodiments, the antibody or antigen binding fragment provided herein comprises a VH comprising the amino acid sequence of SEQ ID NO:800, and a VL comprising the amino acid sequence of SEQ ID NO:801. In some embodiments, the antibody or antigen binding fragment provided herein comprises a VH comprising the amino acid sequence of SEQ ID NO:802, and a VL comprising the amino acid sequence of SEQ ID NO:803. In some embodiments, the antibody or antigen binding fragment provided herein comprises a VH comprising the amino acid sequence of SEQ ID NO:804, and a VL comprising the amino acid sequence of SEQ ID NO:805. In some embodiments, the antibody or antigen binding fragment provided herein comprises a VH comprising the amino acid sequence of SEQ ID NO:834, and a VL comprising the amino acid sequence of SEQ ID NO:835. In some embodiments, the antibody or antigen binding fragment provided herein comprises a VH comprising the amino acid sequence of SEQ ID NO:844, and a VL comprising the amino acid sequence of SEQ ID NO:845. In some embodiments, the antibody or antigen binding fragment provided herein comprises a VH comprising the amino acid sequence of SEQ ID NO:892, and a VL comprising the amino acid sequence of SEQ ID NO:893. In some embodiments, the antibody or antigen binding fragment provided herein comprises a VH comprising the amino acid sequence of SEQ ID NO:894, and a VL comprising the amino acid sequence of SEQ ID NO:895. In some embodiments, the antibody or antigen binding fragment provided herein comprises a VH comprising the amino acid sequence of SEQ ID NO:896, and a VL comprising the amino acid sequence of SEQ ID NO:897. In some embodiments, the antibody or antigen binding fragment provided herein comprises a VH comprising the amino acid sequence of SEQ ID NO:898 and a VL comprising the amino acid sequence of SEQ ID NO:899.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 136, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:137. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:140, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:141. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:146, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:147. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:184, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:185. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:794, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:795. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:796, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:797. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:800, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:801. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:802, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:803. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:804, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:805. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:834, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:835. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:844, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:845. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:892, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:893. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:894, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:895. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:896, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:897. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:898 and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:899. In some embodiments, the antibody or antigen binding fragment provided herein binds to the same epitope as an antibody comprising a VH comprising the amino acid sequence of SEQ ID NO:136, and a VL comprising the amino acid sequence of SEQ ID NO: 137. In some embodiments, the antibody or antigen binding fragment provided herein binds to the same epitope as an antibody comprising a VH comprising the amino acid sequence of SEQ ID NO:140, and a VL comprising the amino acid sequence of SEQ ID NO:141. In some embodiments, the antibody or antigen binding fragment provided herein binds to the same epitope as an antibody comprising a VH comprising the amino acid sequence of SEQ ID NO:146, and a VL comprising the amino acid sequence of SEQ ID NO: 147. In some embodiments, the antibody or antigen binding fragment provided herein binds to the same epitope as an antibody comprising a VH comprising the amino acid sequence of SEQ ID NO:184, and a VL comprising the amino acid sequence of SEQ ID NO: 185. In some embodiments, the antibody or antigen binding fragment provided herein binds to the same epitope as an antibody comprising a VH comprising the amino acid sequence of SEQ ID NO:244, and a VL comprising the amino acid sequence of SEQ ID NO:245. In some embodiments, the antibody or antigen binding fragment provided herein binds to the same epitope as an antibody comprising a VH comprising the amino acid sequence of SEQ ID NO:794, and a VL comprising the amino acid sequence of SEQ ID NO:795. In some embodiments, the antibody or antigen binding fragment provided herein binds to the same epitope as an antibody comprising a VH comprising the amino acid sequence of SEQ ID NO:796, and a VL comprising the amino acid sequence of SEQ ID NO:797. In some embodiments, the antibody or antigen binding fragment provided herein binds to the same epitope as an antibody comprising a VH comprising the amino acid sequence of SEQ ID NO:800, and a VL comprising the amino acid sequence of SEQ ID NO:801. In some embodiments, the antibody or antigen binding fragment provided herein binds to the same epitope as an antibody comprising a VH comprising the amino acid sequence of SEQ ID NO:802, and a VL comprising the amino acid sequence of SEQ ID NO:803. In some embodiments, the antibody or antigen binding fragment provided herein binds to the same epitope as an antibody comprising a VH comprising the amino acid sequence of SEQ ID NO:804, and a VL comprising the amino acid sequence of SEQ ID NO:805. In some embodiments, the antibody or antigen binding fragment provided herein binds to the same epitope as an antibody comprising a VH comprising the amino acid sequence of SEQ ID NO:834, and a VL comprising the amino acid sequence of SEQ ID NO:835. In some embodiments, the antibody or antigen binding fragment provided herein binds to the same epitope as an antibody comprising a VH comprising the amino acid sequence of SEQ ID NO:844, and a VL comprising the amino acid sequence of SEQ ID NO:845. In some embodiments, the antibody or antigen binding fragment provided herein binds to the same epitope as an antibody comprising a VH comprising the amino acid sequence of SEQ ID NO:892, and a VL comprising the amino acid sequence of SEQ ID NO:893. In some embodiments, the antibody or antigen binding fragment provided herein binds to the same epitope as an antibody comprising a VH comprising the amino acid sequence of SEQ ID NO:894, and a VL comprising the amino acid sequence of SEQ ID NO:895. In some embodiments, the antibody or antigen binding fragment provided herein binds to the same epitope as an antibody comprising a VH comprising the amino acid sequence of SEQ ID NO:896, and a VL comprising the amino acid sequence of SEQ ID NO:897. In some embodiments, the antibody or antigen binding fragment provided herein binds to the same epitope as an antibody comprising a VH comprising the amino acid sequence of SEQ ID NO:898 and a VL comprising the amino acid sequence of SEQ ID NO:899.

In some embodiments, the antibody comprises a heavy chain comprising the amino acid sequence of SEQ ID NO:2 and a light chain comprising the amino acid sequence of SEQ ID NO:3. In some embodiments, the antibody comprises a heavy chain comprising the amino acid sequence of SEQ ID NO:6 and a light chain comprising the amino acid sequence of SEQ ID NO:7. In some embodiments, the antibody comprises a heavy chain comprising the amino acid sequence of SEQ ID NO:12 and a light chain comprising the amino acid sequence of SEQ ID NO:13. In some embodiments, the antibody comprises a heavy chain comprising the amino acid sequence of SEQ ID NO:50 and a light chain comprising the amino acid sequence of SEQ ID NO:51. In some embodiments, the antibody comprises a heavy chain comprising the amino acid sequence of SEQ ID NO:110 and a light chain comprising the amino acid sequence of SEQ ID NO:111. In some embodiments, the antibody comprises a heavy chain comprising the amino acid sequence of SEQ ID NO:680 and a light chain comprising the amino acid sequence of SEQ ID NO:681. In some embodiments, the antibody comprises a heavy chain comprising the amino acid sequence of SEQ ID NO:682 and a light chain comprising the amino acid sequence of SEQ ID NO:683. In some embodiments, the antibody comprises a heavy chain comprising the amino acid sequence of SEQ ID NO:686 and a light chain comprising the amino acid sequence of SEQ ID NO:687. In some embodiments, the antibody comprises a heavy chain comprising the amino acid sequence of SEQ ID NO:688 and a light chain comprising the amino acid sequence of SEQ ID NO:689. In some embodiments, the antibody comprises a heavy chain comprising the amino acid sequence of SEQ ID NO:690 and a light chain comprising the amino acid sequence of SEQ ID NO:691. In some embodiments, the antibody comprises a heavy chain comprising the amino acid sequence of SEQ ID NO:720 and a light chain comprising the amino acid sequence of SEQ ID NO:721. In some embodiments, the antibody comprises a heavy chain comprising the amino acid sequence of SEQ ID NO:730 and a light chain comprising the amino acid sequence of SEQ ID NO:731. In some embodiments, the antibody comprises a heavy chain comprising the amino acid sequence of SEQ ID NO:778 and a light chain comprising the amino acid sequence of SEQ ID NO:779. In some embodiments, the antibody comprises a heavy chain comprising the amino acid sequence of SEQ ID NO:780 and a light chain comprising the amino acid sequence of SEQ ID NO:781. In some embodiments, the antibody comprises a heavy chain comprising the amino acid sequence of SEQ ID NO:782 and a light chain comprising the amino acid sequence of SEQ ID NO:783. In some embodiments, the antibody comprises a heavy chain comprising the amino acid sequence of SEQ ID NO:784 and a light chain comprising the amino acid sequence of SEQ ID NO:785.

Thus, in an aspect or embodiment of the invention, including methods and assays and kits provided herein, one or more of the above indicated and provided non binders or weak binders antibodies are included as a control. The non binder or weak binder antibodies may be combined with other antibodies, including antibodies of one or more unique epitope bin, including as a control or to test for true IL-23R antigen binding, or to compare distinct or unique IL-23R epitope binding or for epitope characterization or comparison.

In some embodiments, the IL-23R binding protein is an antibody comprising a particular designated light chain, particularly a consensus or common light chain, such as a common LC sequence, a common VL sequence and/or a set of common light chain CDR1, CDR2 and CDR3 sequences and a unique or distinct heavy chain comprising a unique or distinct HC sequence, VH sequence and/or a unique heavy chain set of CDRs (CDR1, CDR2 and CDR3). This and these antibodies may have unique and different epitope binding (including being grouped in distinct or the same epitope bins) and/or affinity characteristics, such characateristics primarily determined or even solely determined, by their heavy chain sequence(s). As described herein, including in Tables 13, 14 and 15, common light chain sequences and their unique heavy chain components in certain antibodies are detailed.

Antibodies, particularly human anti-human IL-23R antibodies can be characterized as having an identical LC, VL and light chain CDR1, CDR2 and CDR3 sequence and grouped according to the following. The antibody identifier and VH sequence is provided for each relevant antibody. The heavy chain CDRs are provided in the application including in Table 9 and can be determined within the VH sequence by known and available methods and according to antibody standards. There are several groups of antibodies with the antibodies in each group sharing an identical VL, LC and light chain CDR1, CDR2 and CDR3 sequence, but having distinct heavy chain sequences. The antibodies are grouped by light chain consensus:

Group 1—VL sequence SEQ ID NO:1247, or the CDR1, CR2 and CDR3 sequences therein, and VH sequence selected from:
I23RB1 SEQ ID NO:786
I23RB2 SEQ ID NO:788
I23RB3 SEQ ID NO:790
I23RB4 SEQ ID NO:792
I23RB8 SEQ ID NO:800
I23RB9 SEQ ID NO:802
I23RB268 SEQ ID NO:824
I23RB270 SEQ ID NO:828
I23RB291 SEQ ID NO:870
   Group2—VL sequence SEQ ID NO:1248, or the CDR1, CR2 and CDR3 sequences therein, and VH sequence selected from:
I23RB5 SEQ ID NO:794
I23RB266 SEQ ID NO:820
I23RB271 SEQ ID NO:830
I23RB273 SEQ ID NO:834
I23RB274 SEQ ID NO:836
I23RB275 SEQ ID NO:838
I23RB276 SEQ ID NO:840
I23RB277 SEQ ID NO:842
I23RB279 SEQ ID NO:846
I23RB281 SEQ ID NO:850
I23RB282 SEQ ID NO:852
I23RB284 SEQ ID NO:856
I23RB286 SEQ ID NO:858
I23RB289 SEQ ID NO:866
I23RB290 SEQ ID NO:868
I23RB292 SEQ ID NO:872
   Group3—VL sequence SEQ ID NO:1249, or the CDR1, CR2 and CDR3 sequences therein, and VH sequence selected from:
I23RB6 SEQ ID NO:796
I23RB10 SEQ ID NO:804
   Group4—VL sequence SEQ ID NO:1250, or the CDR1, CR2 and CDR3 sequences therein, and VH sequence selected from:
I23RB259 SEQ ID NO:806
I23RB316 SEQ ID NO:880
   Group5—VL sequence SEQ ID NO:1251, or the CDR1, CR2 and CDR3 sequences therein, and VH sequence selected from:
I23RB267 SEQ ID NO:822
I23RB269 SEQ ID NO:826
I23RB272 SEQ ID NO:832
I23RB278 SEQ ID NO:844
I23RB280 SEQ ID NO:848
I23RB283 SEQ ID NO:854
I23RB287 SEQ ID NO:862
I23RB288 SEQ ID NO:864

For each of the above groups and particular antibodies, the heavy chain sequences HC, VH and Heavy chain CDR1, CDR2 and CDR3 contribute to and have responsible significance for epitope binding and affinity distinctions between and/or among the antibodes. The heavy chain in each instance determines the binding and affinity or uniqueness and epitope specificity and epitope differential binding of the antibodies. Heavy chain domain only or VHH antibodies based on the heavy chain sequence—either of the VH sequence, HC sequence, or comprising the CDR1, CDR2 and CDR3 sequences of the heavy chain—of any of these grouped antibodies may be of particular interest, use and application and are an aspect and embodiment provided and described herein.In some embodiments, the IL-23R binding protein is a monoclonal antibody, including a mouse, chimeric, humanized or human antibody. In some embodiments, the anti-IL-23R antibody is an antibody fragment, e.g., a scFv. In some embodiments, the IL-23R binding protein is a fusion protein comprising the anti-IL-23R antibody provided herein. In other embodiments, the IL-23R binding protein is a multispecific antibody comprising the anti-IL-23R antibody provided herein. Other exemplary IL-23R binding molecules are described in more detail in the following sections.

In some embodiments, the anti-IL-23R antibody or antigen binding protein according to any of the above embodiments may incorporate any of the features, singly or in combination, as described below.

### Antibody Fragments

As used herein, the term “antibody” also includes various antibody fragments thereof. Antibodies provided herein include, but are not limited to, immunoglobulin molecules and immunologically active portions of immunoglobulin molecules. The immunoglobulin molecules provided herein can be of any class (e.g., IgG, IgE, IgM, IgD, and IgA) or any subclass (e.g., IgG1, IgG2, IgG3, IgG4, IgA1, and IgA2) of immunoglobulin molecule. In some embodiments, the antibody is an IgG antibody. In some embodiments, the IgG antibody is an IgG1 antibody. In some embodiments, the IgG antibody is an IgG2, IgG3, or IgG4 antibody.

Variants and derivatives of antibodies include antibody functional fragments or antigen binding fragments that retain the ability to bind to an antigen. Exemplary functional fragments or antigen binding fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)2 (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of, e.g., 10-25 amino acids); a disulfide-linked Fv, or dsFv (e.g., the variable, antigen-binding determinative region of a single light and heavy chain of an antibody linked together by a disulfide bond); a VHH or nanobody (e.g. the variable, antigen-determinative region or an antigen binding domain of the heavy chain of an antibody only); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); a triabody (e.g., a trimerized scFv, formed in a manner similar to a diabody, but in which three antigen-binding domains are created in a single complex; the three antigen binding domains may be directed towards the same or different epitopes); a tetrabody (e.g., a tetramerized scFv, formed in a manner similar to a diabody, but in which four antigen-binding domains are created in a single complex; the four antigen binding domains may be directed towards the same or different epitopes); and other non-full length portions of heavy and/or light chains alone or in any combination.

Nanobodies or VHH antibodies, are single domain (Heavy chain variable region VHH) antibodies (Ward, E. S. et al., Nature 341, 544-546 (1989)). Single domain antibodies were initially isolated firm camelid animals and have been designated interchangeably as camelid antibodies, nanobodies or VHH. A VHH antibody corresponds to the variable region of an antibody heavy chain and has a very small size of around 15 kDa—hence the name “nanobody”, The advantage of these antibody-derived molecules is their small size which can enable their binding to hidden epitopes not accessible to whole antibodies. In the context of therapeutic applications, a small molecular weight also means an efficient penetration and fast clearance. Both scFv and VHH nanobodies can be linked to the Fc fragment of the desired species and keep their specificity and binding properties and are then termed minibody.

Nanobodies are small, low molecular weight, single-domain, heavy-chain only antibody. Owing to its smaller size, genes of these proteins can be readily cloned and manipulated to present on plasmids or in integrated form, expression vector, etc. Therefore, by using molecular cloning techniques, nanobodies against various antigens or against distinct epitopes on a single antigen can be presented, even on a single or multiple constructs, and be provided in an assay, to cells in an in vitro or in vivo study, to a target region or to the systemic circulation.

Antibody(ies) comprising linked nanobodies, such as multimeric and bi-specific versions are included in embodiments of the invention. Thus, two or more nanobodies or sequences encoding two or more nanobodies can be covalently linked, gthrough a linker sequence or any such other recognized and applicable means, to form a bispecific or multimeric form of the nanobody(ies). In a an embodiment, two distinct nanobodies are linked. In an embodiment a single nanobody is multimerized through linkage, which may have applicability to increase binding, avidity, affinity. In an embodiment, two or more nanobodies, including nanobodies directed against distinct IL-23R protein epitopes (such as epitope bins) are linked.

Described and provided herein are numerous IL-23R antibodies, particularly human phage library derived antibodies which have identical light chain VL and LCDR1, LCDR2 and LCDR3 sequences and unique heavy chain sequences, including unique and distinct HCDR1, HCDR2 and HCDR3 sequences. Despite identical light chains, these antibodies exhibit distinct binding affinities and binding characteristics with regard to IL-23R, including in instances recognizing distinct epitopes, being placed in distinct epitope bins in the analysis provided herein, The heavy chain sequences, particularly the VHH sequences, and the heavy chain CDR1, CDR2 and CDR3 sequences of these antibodies thus are the distinctive characteristic of these antibodies and significantly contribute to or are responsible for their distinctions. These antibodies demonstrate the importance of heavy chain sequence, particularly heavy chain antigen binding domains. Further, the relevance of VHH antibodies or nanobodies as useful and applicable is also demonstrated. In a particular embodiment, VHH antibodies comprising the VH sequences or comprising one or more CDRs set forth in SEQ ID NOs:136-269, 786-899 are provided herein. In one embodiment, VHH antibodies comprising the VH sequences or comprising one or more CDRs set forth in SEQ ID NOs: 786-899 are provided herein. In one embodiment, VHH antibodies comprising the VH sequences or comprising one or more CDRs set forth in SEQ ID NOs: 786-797, 800-807, 820-873 or 880-881 are provided herein.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. For example, Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., 1992, Bio/Technology 10:163-67). According to another approach, F(ab′)2 fragments can be isolated directly from recombinant host cell culture. Fab and F(ab′)2 fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of a scFv (See, e.g., Borrebaeck ed., supra). The antibody fragment may also be a “linear antibody,” for example, as described in the references cited above. Such linear antibodies may be monospecific or multi-specific, such as bispecific.

### Humanized Antibodies

The antibodies described herein include humanized antibodies. Humanized antibodies, such as the humanized antibodies disclosed herein can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, Molecular Immunology 28(4/5):489-498 (1991); Studnicka et al., Protein Engineering 7(6):805-814 (1994); and Roguska et al., PNAS 91:969-973 (1994)), chain shuffling (U.S. Pat. No. 5,565,332), and techniques disclosed in, e.g., U.S. Pat. Nos. 6,407,213, 5,766,886, WO 9317105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s- 5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety.

In some embodiments, antibodies provided herein can be humanized antibodies that bind to IL-23R, including human IL-23R. For example, humanized antibodies of the present disclosure may comprise one or more CDRs set forth in SEQ ID NOs:136-269, 786-899. In some embodiments, humanized antibodies may comprise one or more CDRs set forth in the mouse antibodies provided herein, including the antibodies derived by immunization including SEQ ID NO:s 136-269, and in SEQ ID NOs:784-785. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., Nature 321:522-25 (1986); Riechmann et al., Nature 332:323-27 (1988); and Verhoeyen et al., Science 239:1534-36 (1988)), by substituting hypervariable region sequences for the corresponding sequences of a human antibody. In a specific embodiment, humanization of the antibody provided herein is performed as described in Section 6 below.

In some cases, the humanized antibodies are constructed by CDR grafting, in which the amino acid sequences of the CDRs of the parent non-human antibody are grafted onto a human antibody framework. For example, Padlan et al. determined that only about one third of the residues in the CDRs actually contact the antigen, and termed these the “specificity determining residues,” or SDRs (Padlan et al., FASEB J. 9:133-39 (1995)). In the technique of SDR grafting, only the SDR residues are grafted onto the human antibody framework (see, e.g., Kashmiri et al., Methods 36:25-34 (2005)).

The choice of human variable domains to be used in making the humanized antibodies can be important to reduce antigenicity. For example, according to the so-called “best-fit” method, the sequence of the variable domain of a non-human antibody is screened against the entire library of known human variable-domain sequences. The human sequence that is closest to that of the non-human antibody may be selected as the human framework for the humanized antibody (Sims et al., J. Immunol. 151:2296-308 (1993); and Chothia et al., J. Mol. Biol. 196:901-17 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA 89:4285-89 (1992); and Presta et al., J. Immunol. 151:2623-32 (1993)). In some cases, the framework is derived from the consensus sequences of the most abundant human subclasses, V_{L}6 subgroup I (V_{L}6I) and VH subgroup III (V_{H}III). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., J. Immunol. 169:1119-25 (2002)).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13:819-24 (2002)), Modeller (Sali and Blundell, J. Mol. Biol. 234:779-815 (1993)), and Swiss PDB Viewer (Guex and Peitsch, Electrophoresis 18:2714-23 (1997)). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., Mol. Immunol. 44:1986-98 (2007)).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, Nat. Biotechnol. 23:1105-16 (2005); Dufner et al., Trends Biotechnol. 24:523-29 (2006); Feldhaus et al., Nat. Biotechnol. 21:163-70 (2003); and Schlapschy et al., Protein Eng. Des. Sel. 17:847-60 (2004)).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, J. Mol. Biol. 224:487-99 (1992)), or from the more limited set of target residues identified by Baca et al. J. Biol. Chem. 272:10678-84 (1997).

In FR shuffling, whole FRs are combined with the non-human CDRs instead of creating combinatorial libraries of selected residue variants (see, e.g., Dall'Acqua et al., Methods 36:43-60 (2005)). A one-step FR shuffling process may be used. Such a process has been shown to be efficient, as the resulting antibodies exhibited improved biochemical and physicochemical properties including enhanced expression, increased affinity, and thermal stability (see, e.g., Damschroder et al., Mol. Immunol. 44:3049-60 (2007)).

The “humaneering” method is based on experimental identification of essential minimum specificity determinants (MSDs) and is based on sequential replacement of non-human fragments into libraries of human FRs and assessment of binding. This methodology typically results in epitope retention and identification of antibodies from multiple subclasses with distinct human V-segment CDRs.

The “human engineering” method involves altering a non-human antibody or antibody fragment by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering 7:805-14 (1994); U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody.

A deimmunized antibody is an antibody in which T-cell epitopes have been removed. Methods for making deimmunized antibodies have been described. See, e.g., Jones et al., Methods Mol Biol. 525:405-23 (2009), xiv, and De Groot et al., Cell. Immunol. 244:148-153(2006)). Deimmunized antibodies comprise T-cell epitope-depleted variable regions and human constant regions. Briefly, variable regions of an antibody are cloned and T-cell epitopes are subsequently identified by testing overlapping peptides derived from the variable regions of the antibody in a T cell proliferation assay. T cell epitopes are identified via in silico methods to identify peptide binding to human MHC class II. Mutations are introduced in the variable regions to abrogate binding to human MHC class II. Mutated variable regions are then utilized to generate the deimmunized antibody.

### Antibody Variants

In some embodiments, amino acid sequence modification(s) of the antibodies that bind to IL-23R described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the antibodies that bind to IL-23R described herein, it is contemplated that variants of the antibodies that bind to IL-23R described herein can be prepared. For example, antibody variants can be prepared by introducing appropriate nucleotide changes into the encoding DNA, and/or by synthesis of the desired antibody or polypeptide. Those skilled in the art who appreciate that amino acid changes may alter post-translational processes of the antibody.

### Chemical Modifications

In some embodiments, the antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, or conjugation to one or more immunoglobulin domains (e.g., Fc or a portion of an Fc). Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

When the antibody provided herein is fused to an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the binding molecules provided herein may be made in order to create variants with certain improved properties.

In other embodiments, when the antibody provided herein is fused to an Fc region, antibody variants provided herein may have a carbohydrate structure that lacks fucose attached (directly or indirectly) to said Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 and US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108; and WO 2004/056312, and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

The binding molecules comprising an antibody provided herein are further provided with bisected oligosaccharides, e.g., in which a biantennary oligosaccharide attached to the Fc region is bisected by GlcNAc. Such variants may have reduced fucosylation and/or improved ADCC function. Examples of such variants are described, e.g., in WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such variants may have improved CDC function. Such variants are described, e.g., in WO 1997/30087; WO 1998/58964; and WO 1999/22764.

In molecules that comprise the present antibody and an Fc region, one or more amino acid modifications may be introduced into the Fc region, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the present application contemplates variants that possesses some but not all effector functions, which make it a desirable candidate for applications in which the half-life of the binding molecule in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the binding molecule lacks FcγR binding (hence likely lacking ADCC activity), but retains FcRn binding ability. Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96© non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Binding molecules with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, a variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Binding molecules with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those molecules comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

In some embodiments, it may be desirable to create cysteine engineered antibodies, in which one or more residues of an antibody are substituted with cysteine residues. In some embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein.

### Substitutions, Deletions, or Insertions

Variations may be a substitution, deletion, or insertion of one or more codons encoding the antibody or polypeptide that results in a change in the amino acid sequence as compared with the original antibody or polypeptide. Sites of interest for substitutional mutagenesis include the CDRs and FRs.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Antibodies generated by conservative amino acid substitutions are included in the present disclosure. In a conservative amino acid substitution, an amino acid residue is replaced with an amino acid residue having a side chain with a similar charge. As described above, families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. Conservative (e.g., within an amino acid group with similar properties and/or side chains) substitutions may be made, so as to maintain or not significantly change the properties. Exemplary substitutions are shown in Table 2 below.

TABLE 2
Amino Acid Substitutions

| Original | Exemplary |
|---|---|
| Residue | Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; |
| | Norleucine |
| Leu (L) | Norleucine; Ile; Val; Met; Ala; |
| | Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; |
| | Norleucine |

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. For example, any cysteine residue not involved in maintaining the proper conformation of the antibody also may be substituted, for example, with another amino acid, such as alanine or serine, to improve the oxidative stability of the molecule and to prevent aberrant crosslinking. Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant antibody or fragment thereof being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. More detailed description regarding affinity maturation is provided in the section below.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments of the variant antibody sequences provided herein, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, Science, 244:1081-1085 (1989). In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

The variations can be made using methods known in the art such as oligonucleotide-mediated (site-directed) mutagenesis, alanine scanning, and PCR mutagenesis. Site-directed mutagenesis (see, e.g., Carter, Biochem J. 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the antibody variant DNA.

In vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, Methods. Mol. Biol. 178:1-37 (2002); and Bradbury and Marks, J. Immunol. Methods 290:29-49 (2004).

In a yeast display system (see, e.g., Boder et al., Nat. Biotech. 15:553-57 (1997); and Chao et al., Nat. Protocols 1:755-68 (2006)), the antibody may be fused to the adhesion subunit of the yeast agglutinin protein Aga2p, which attaches to the yeast cell wall through disulfide bonds to Agalp. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the single chain antibody (e.g., scFv). Expression has been shown to correlate with the stability of the displayed protein, and thus antibodies can be selected for improved stability as well as affinity (see, e.g., Shusta et al., J. Mol. Biol. 292:949-56 (1999)). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10¹⁴ in size (see, e.g., U.S. Pat. Publication 2003/0186374; and Blaise et al., Gene 342:211-18 (2004)).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., Nucleic Acids Res. 34:e127 (2006)). In mRNA display, a covalent bond between antibody and mRNA is established using puromycin as an adaptor molecule (Wilson et al., Proc. Natl. Acad. Sci. USA 98:3750-55 (2001)).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

In some embodiments, mammalian display systems may be used.

Diversity may also be introduced into the CDRs of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., J. Biol. Chem. 280:607-17 (2005)) or residues suspected of affecting affinity on experimental basis or structural reasons. Diversity may also be introduced by replacement of regions that are naturally diverse via DNA shuffling or similar techniques (see, e.g., Lu et al., J. Biol. Chem. 278:43496-507 (2003); U.S. Pat. Nos. 5,565,332 and 6,989,250). Alternative techniques target hypervariable loops extending into framework-region residues (see, e.g., Bond et al., J. Mol. Biol. 348:699-709 (2005)) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, antibodies can be immobilized onto solid supports, columns, pins, or cellulose/poly (vinylidene fluoride) membranes/other filters, expressed on host cells affixed to adsorption plates or used in cell sorting, or conjugated to biotin for capture with streptavidin-coated beads or used in any other method for panning display libraries.

For review of in vitro affinity maturation methods, see, e.g., Hoogenboom, Nature Biotechnology 23:1105-16 (2005); Quiroz and Sinclair, Revista Ingeneria Biomedia 4:39-51 (2010); and references therein.

### Modifications of Antibodies

Covalent modifications of antibodies are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody included within the scope of this present disclosure include altering the native glycosylation pattern of the antibody or polypeptide as described above (see, e.g., Beck et al., Curr. Pharm. Biotechnol. 9:482-501 (2008); and Walsh, Drug Discov. Today 15:773-80 (2010)), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337. The antibody that binds to IL-23R of the disclosure may also be genetically fused or conjugated to one or more immunoglobulin constant regions or portions thereof (e.g., Fc) to extend half-life and/or to impart known Fc-mediated effector functions.

The antibody that binds to IL-23R of the present disclosure may also be modified to form chimeric molecules comprising the antibody that binds to IL-23R fused to another, heterologous polypeptide or amino acid sequence, for example, an epitope tag (see, e.g., Terpe, Appl. Microbiol. Biotechnol. 60:523-33 (2003)) or the Fc region of an IgG molecule (see, e.g., Aruffo, Antibody Fusion Proteins 221-42 (Chamow and Ashkenazi eds., 1999)).

Also provided herein are fusion proteins comprising the antibody that binds to IL-23R of the disclosure and a heterologous polypeptide. In some embodiments, the heterologous polypeptide to which the antibody is genetically fused or chemically conjugated is useful for targeting the antibody to cells having cell surface-expressed IL-23R.

Also provided herein are panels of antibodies that bind to an IL-23R antigen. In specific embodiments, the panels of antibodies have different association rates, different dissociation rates, different affinities for an IL-23R antigen, and/or different specificities for an IL-23R antigen. In some embodiments, the panels comprise or consist of about 10 to about 1000 antibodies or more. Panels of antibodies can be used, for example, in 96-well or 384-well plates, for assays such as ELISAs.

### Other Binding Molecules Comprising the Antibodies

In another aspect, provided herein is a binding molecule comprising an anti-IL-23R antibody provided herein. In some embodiments, an antibody against IL-23R provided herein is part of other binding molecules. Exemplary binding molecules of the present disclosure are described herein.

### Fusion Protein

In various embodiments, the antibody provided herein can be genetically fused or chemically conjugated to another agent, for example, protein-based entities. The antibody may be chemically-conjugated to the agent, or otherwise non-covalently conjugated to the agent. The agent can be a peptide or antibody (or a fragment thereof).

Thus, in some embodiments, provided herein are antibodies that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, about 100, about 150, about 200, about 250, about 300, about 350, about 400, about 450 or about 500 amino acids, or over 500 amino acids) to generate fusion proteins, as well as uses thereof. In particular, provided herein are fusion proteins comprising an antigen binding fragment of the antibody provided herein (e.g., CDR1, CDR2, and/or CDR3) and a heterologous protein, polypeptide, or peptide.

Moreover, antibodies provided herein can be fused to marker or “tag” sequences, such as a peptide, to facilitate purification. In specific embodiments, the marker or tag amino acid sequence is a hexa-histidine peptide, hemagglutinin (“HA”) tag, and “FLAG” tag.

Methods for fusing or conjugating moieties (including polypeptides) to antibodies are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., Immunol. Rev. 62:119-58 (1982); U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88: 10535-39 (1991); Traunecker et al., Nature, 331:84-86 (1988); Zheng et al., J. Immunol. 154:5590-600 (1995); and Vil et al., Proc. Natl. Acad. Sci. USA 89:11337-41 (1992)).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the antibodies as provided herein, including, for example, antibodies with higher affinities and lower dissociation rates (see, e.g., U.S. Pat. Nos. 5,605,793; 5,811,238; 5,830,721; 5,834,252; and U.S. Pat. No. 5,837,458; Patten et al., Curr. Opinion Biotechnol. 8:724-33 (1997); Harayama, Trends Biotechnol. 16(2):76-82 (1998); Hansson et al., J. Mol. Biol. 287:265-76 (1999); and Lorenzo and Blasco, Biotechniques 24(2):308-13 (1998)). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

In some embodiments, an antibody provided herein is conjugated to a second antibody to form an antibody heteroconjugate.

In various embodiments, the antibody is genetically fused to the agent. Genetic fusion may be accomplished by placing a linker (e.g., a polypeptide) between the antibody and the agent. The linker may be a flexible linker.

In various embodiments, the antibody is genetically conjugated to a therapeutic molecule, with a hinge region linking the antibody to the therapeutic molecule.

Also provided herein are methods for making the various fusion proteins provided herein. The various methods described in Section 5.4 may also be utilized to make the fusion proteins provided herein.

In a specific embodiment, the fusion protein provided herein is recombinantly expressed. Recombinant expression of a fusion protein provided herein may require construction of an expression vector containing a polynucleotide that encodes the protein or a fragment thereof. Once a polynucleotide encoding a protein provided herein or a fragment thereof has been obtained, the vector for the production of the molecule may be produced by recombinant DNA technology using techniques well-known in the art. Thus, methods for preparing a protein by expressing a polynucleotide containing an encoding nucleotide sequence are described herein. Methods which are well known to those skilled in the art can be used to construct expression vectors containing coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding a fusion protein provided herein, or a fragment thereof, or a CDR, operably linked to a promoter.

The expression vector can be transferred to a host cell by conventional techniques and the transfected cells are then cultured by conventional techniques to produce a fusion protein provided herein. Thus, also provided herein are host cells containing a polynucleotide encoding a fusion protein provided herein or fragments thereof operably linked to a heterologous promoter.

A variety of host-expression vector systems may be utilized to express the fusion protein provided herein. Such host-expression systems represent vehicles by which the coding sequences of interest may be produced and subsequently purified, but also represent cells which may, when transformed or transfected with the appropriate nucleotide coding sequences, express a fusion protein provided herein in situ. These include but are not limited to microorganisms such as bacteria (e.g., E. coli and B. subtilis) transformed with recombinant bacteriophage DNA, plasmid DNA or cosmid DNA expression vectors containing coding sequences; yeast (e.g., Saccharomyces Pichia) transformed with recombinant yeast expression vectors containing coding sequences; insect cell systems infected with recombinant virus expression vectors (e.g., baculovirus) containing coding sequences; plant cell systems infected with recombinant virus expression vectors (e.g., cauliflower mosaic virus, CaMV, tobacco mosaic virus, TMV) or transformed with recombinant plasmid expression vectors (e.g., Ti plasmid) containing coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or, eukaryotic cells, especially for the expression of whole recombinant antibody molecule, can be used for the expression of a recombinant fusion protein. For example, mammalian cells such as Chinese hamster ovary cells (CHO), in conjunction with a vector such as the major intermediate early gene promoter element from human cytomegalovirus is an effective expression system for antibodies or variants thereof. In a specific embodiment, the expression of nucleotide sequences encoding the fusion proteins provided herein is regulated by a constitutive promoter, inducible promoter or tissue specific promoter.

In bacterial systems, a number of expression vectors may be advantageously selected depending upon the use intended for the fusion protein being expressed. For example, when a large quantity of such a fusion protein is to be produced, for the generation of pharmaceutical compositions of a fusion protein, vectors which direct the expression of high levels of fusion protein products that are readily purified may be desirable. Such vectors include, but are not limited to, the E. coli expression vector pUR278 (Ruther et al., EMBO 12:1791 (1983)), in which the coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the fusion protein in infected hosts (e.g., see Logan & Shenk, Proc. Natl. Acad. Sci. USA 8 1:355-359 (1984)). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT20 and T47D, NS0 (a murine myeloma cell line that does not endogenously produce any immunoglobulin chains), CRL7030 and HsS78Bst cells.

For long-term, high-yield production of recombinant proteins, stable expression can be utilized. For example, cell lines which stably express the fusion proteins may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the fusion protein. Such engineered cell lines may be particularly useful in screening and evaluation of compositions that interact directly or indirectly with the binding molecule.

A number of selection systems may be used, including but not limited to, the herpes simplex virus thymidine kinase (Wigler et al., Cell 11:223 (1977)), hypoxanthineguanine phosphoribosyltransferase (Szybalska & Szybalski, Proc. Natl. Acad. Sci. USA 48:202 (1992)), and adenine phosphoribosyltransferase (Lowy et al., Cell 22:8-17 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIB TECH 11(5):155-2 15 (1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., J. Mol. Biol. 150:1 (1981), which are incorporated by reference herein in their entireties.

The expression level of a fusion protein can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing a fusion protein is amplifiable, increase in the level of inhibitor present in culture of host cell will increase the number of copies of the marker gene. Since the amplified region is associated with the fusion protein gene, production of the fusion protein will also increase (Crouse et al., Mol. Cell. Biol. 3:257 (1983)).

The host cell may be co-transfected with multiple expression vectors provided herein. The vectors may contain identical selectable markers which enable equal expression of respective encoding polypeptides. Alternatively, a single vector may be used which encodes, and is capable of expressing multiple polypeptides. The coding sequences may comprise cDNA or genomic DNA.

Once a fusion protein provided herein has been produced by recombinant expression, it may be purified by any method known in the art for purification of a polypeptide (e.g., an immunoglobulin molecule), for example, by chromatography (e.g., ion exchange, affinity, particularly by affinity for the specific antigen after Protein A, sizing column chromatography, and Kappa select affinity chromatography), centrifugation, differential solubility, or by any other standard technique for the purification of proteins. Further, the fusion protein molecules provided herein can be fused to heterologous polypeptide sequences described herein or otherwise known in the art to facilitate purification.

### Immunoconjugates

In some embodiments, the present disclosure also provides immunoconjugates comprising any of the anti-IL-23R antibodies described herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some embodiments, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natd. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In some embodiments, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments, an immunoconjugate comprises an antibody as described herein conjugated to a radioactive atom to form a radioconjugate. A variety of radioactive isotopes are available for the production of radioconjugates. Examples include At²¹¹, I³¹, I²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

The linker may be a “cleavable linker” facilitating release of the conjugated agent in the cell, but non-cleavable linkers are also contemplated herein. Linkers for use in the conjugates of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers, thioether linkers, or hydrophilic linkers designed to evade multidrug transporter-mediated resistance.

The immunuoconjugates or ADCs herein contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

In other embodiments, antibodies provided herein are conjugated or recombinantly fused, e.g., to a diagnostic molecule. Such diagnosis and detection can be accomplished, for example, by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin or avidin/biotin; fluorescent materials, such as, but not limited to, umbelliferone, fluorescein, fluorescein isothiocynate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, 225 Acγ-emitting, Auger-emitting, β-emitting, an alpha-emitting or positron-emitting radioactive isotope. In some embodiments, the conjugated antibodies are used for screening purpose(s). In some embodiments, the screening method is high throughput screening with a library of the conjugated antibodies. In some embodiments, the conjugated antibodies are used to mark or label a cell or cellular target or antigen for diagnostic, screening, targeting, isolation, quantification or other such purposes or applications. In some embodiments, unique or distinct antibodies, including antibodies conjugated or fused to different diagnostic molecules may be utilized in combination in one or more such application, screening method, etc.

### Polynucleotides

In certain embodiments, the disclosure provides polynucleotides that encode the present antibodies that bind to IL-23R and fusion proteins comprising the antibodies that bind to IL-23R described herein. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the antibody that binds IL-23R of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding the antibody that binds IL-23R of the disclosure. As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

Also provided are vectors comprising the nucleic acid molecules described herein. In an embodiment, the nucleic acid molecules can be incorporated into a recombinant expression vector. The present disclosure provides recombinant expression vectors comprising any of the nucleic acids of the disclosure. As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole; however, parts of the vectors can be naturally-occurring. The described recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. The non-naturally occurring or altered nucleotides or internucleotide linkages do not hinder the transcription or replication of the vector.

In an embodiment, the recombinant expression vector of the disclosure can be any suitable recombinant expression vector, and can be used to transform or transfect any suitable host. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Examples of plant expression vectors include pBI01, pBI01.2, pBIl21, pBI101.3, and pBIN19 (Clontech). Examples of animal expression vectors include pEUK-Cl, pMAM, and pMAMneo (Clontech). The recombinant expression vector may be a viral vector, e.g., a retroviral vector, e.g., a gamma retroviral vector.

In an embodiment, the recombinant expression vectors are prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, SV40, 2μ plasmid, λ, bovine papilloma virus, and the like.

The recombinant expression vector may comprise regulatory sequences, such as transcription and translation initiation and termination codons, which are specific to the type of host (e.g., bacterium, plant, fungus, or animal) into which the vector is to be introduced, as appropriate, and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the described expression vectors include, for instance, neomycin/G418 resistance genes, histidinol x resistance genes, histidinol resistance genes, tetracycline resistance genes, and ampicillin resistance genes.

The recombinant expression vector can comprise a native or normative promoter operably linked to the nucleotide sequence of the disclosure. The selection of promoters, e.g., strong, weak, tissue-specific, inducible and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an RSV promoter, an SV40 promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

In certain embodiments, a polynucleotide is isolated. In certain embodiments, a polynucleotide is substantially pure.

Also provided are host cells comprising the nucleic acid molecules described herein. The host cell may be any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5a, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, Saos, PC12, SP2/0 (American Type Culture Collection (ATCC), Manassas, VA, CRL-1581), NS0 (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATCC CRL-TIB-196). Other useful cell lines include those derived from Chinese Hamster Ovary (CHO) cells such as CHO-K1SV (Lonza Biologics, Walkersville, MD), CHO-K1 (ATCC CRL-61) or DG44.

### Preparation of Antibodies and Method of Making

Methods of preparing antibodies have been described. See, e.g., E1s Pardon et al, Nature Protocol, 9(3): 674 (2014). Antibodies (such as scFv fragments) may be obtained using methods known in the art such as by immunizing a Camelid species (such as camel or llama) and obtaining hybridomas therefrom, or by cloning a library of antibodies using molecular biology techniques known in the art and subsequent selection by ELISA with individual clones of unselected libraries or by using phage display.

Antibodies provided herein may be produced by culturing cells transformed or transfected with a vector containing an antibody-encoding nucleic acid. Polynucleotide sequences encoding polypeptide components of the antibody of the present disclosure can be obtained using standard recombinant techniques. Desired polynucleotide sequences may be isolated and sequenced from antibody producing cells such as hybridomas cells or B cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present disclosure. Selection of an appropriate vector will depend mainly on the size of the nucleic acids to be inserted into the vector and the particular host cell to be transformed with the vector. Host cells suitable for expressing antibodies of the present disclosure include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian host cell lines. Host cells are transformed with the above-described expression vectors and cultured in conventional nutrient media modified as appropriate for inducing promoters, selecting transformants, or amplifying the genes encoding the desired sequences. Antibodies produced by the host cells are purified using standard protein purification methods as known in the art.

Methods for antibody production including vector construction, expression, and purification are further described in Pluckthun et al., Antibody Engineering: Producing antibodies in Escherichia coli: From PCR to fermentation 203-52 (McCafferty et al. eds., 1996); Kwong and Rader, E. coli Expression and Purification of Fab Antibody Fragments, in Current Protocols in Protein Science (2009); Tachibana and Takekoshi, Production of Antibody Fab Fragments in Escherichia coli, in Antibody Expression and Production (Al-Rubeai ed., 2011); and Therapeutic Monoclonal Antibodies: From Bench to Clinic (An ed., 2009).

It is, of course, contemplated that alternative methods, which are well known in the art, may be employed to prepare anti-IL-23R antibodies. For instance, the appropriate amino acid sequence, or portions thereof, may be produced by direct peptide synthesis using solid-phase techniques (see, e.g., Stewart et al., Solid-Phase Peptide Synthesis (1969); and Merrifield, J. Am. Chem. Soc. 85:2149-54 (1963)). In vitro protein synthesis may be performed using manual techniques or by automation. Various portions of the anti-IL-23R antibody may be chemically synthesized separately and combined using chemical or enzymatic methods to produce the desired anti-IL-23R antibody. Alternatively, antibodies may be purified from cells or bodily fluids, such as milk, of a transgenic animal engineered to express the antibody, as disclosed, for example, in U.S. Pat. Nos. 5,545,807 and 5,827,690.

### Polyclonal Antibodies

Polyclonal antibodies are generally raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

For example, the animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant cell culture as protein fusions. Also, aggregating agents such as alum are suitable to enhance the immune response.

### Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, an appropriate host animal is immunized to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986).

The immunizing agent will typically include the antigenic protein or a fusion variant thereof. Goding, Monoclonal Antibodies: Principles and Practice, Academic Press (1986), pp. 59-103. Immortalized cell lines are usually transformed mammalian cells. The hybridoma cells thus prepared are seeded and grown in a suitable culture medium that preferably contains one or more substances that inhibit the growth or survival of the unfused, parental myeloma cells. Preferred immortalized myeloma cells are those that fuse efficiently, support stable high-level production of antibody by the selected antibody-producing cells, and are sensitive to a medium such as HAT medium.

Culture medium in which hybridoma cells are growing is assayed for production of monoclonal antibodies directed against the antigen. The culture medium in which the hybridoma cells are cultured can be assayed for the presence of monoclonal antibodies directed against the desired antigen. Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Pliiuckthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991). Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence (U.S. Pat. No. 4,816,567; Morrison, et al., Proc. Natl Acad. Sci. USA, 81:6851 (1984)), or by covalently joining to the coding sequence all or part of the coding sequence for a non-immunoglobulin polypeptide. Such non-immunoglobulin polypeptides can be substituted to create a chimeric bivalent antibody comprising one antigen-combining site having specificity for an antigen and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Recombinant Production in Prokarvotic Cells

Polynucleic acid sequences encoding the antibodies of the present disclosure can be obtained using standard recombinant techniques. Desired polynucleic acid sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present disclosure. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to, an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector which can be used to transform susceptible host cells such as E. coli LE392.

The expression vector of the present application may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the present antibody by removing the promoter from the source DNA via restriction enzyme digestion and inserting the isolated promoter sequence into the vector of the present application. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleic acid sequences have been published, thereby enabling a skilled worker operably to ligate them to cistrons encoding the target peptide (Siebenlist et al. Cell 20: 269 (1980)) using linkers or adaptors to supply any required restriction sites.

In one aspect, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence can be substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

In some embodiments, the production of the antibodies according to the present disclosure can occur in the cytoplasm of the host cell, and therefore does not require the presence of secretion signal sequences within each cistron. Certain host strains (e.g., the E. coli trxB⁻ strains) provide cytoplasm conditions that are favorable for disulfide bond formation, thereby permitting proper folding and assembly of expressed protein subunits.

Prokaryotic host cells suitable for expressing the antibodies of the present disclosure include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In one embodiment, E. coli cells are used as hosts. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A(nmpc-fepE) degP41 kan^{R} (U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon.

Typically the host cell should secrete minimal amounts of proteolytic enzymes, and additional protease inhibitors may desirably be incorporated in the cell culture.

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the antibodies of the present application are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol. The prokaryotic host cells are cultured at suitable temperatures and pHs.

If an inducible promoter is used in the expression vector of the present application, protein expression is induced under conditions suitable for the activation of the promoter. In one aspect of the present application, PhoA promoters are used for controlling transcription of the polypeptides. Accordingly, the transformed host cells are cultured in a phosphate-limiting medium for induction. In some embodiments, the phosphate-limiting medium is the C.R.A.P medium (see, e.g., Simmons et al., J. Immunol. Methods 263:133-147 (2002)). A variety of other inducers may be used, according to the vector construct employed, as is known in the art.

The expressed antibodies of the present disclosure are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantity by a fermentation process. Various large-scale fed-batch fermentation procedures are available for production of recombinant proteins. To improve the production yield and quality of the antibodies of the present disclosure, various fermentation conditions can be modified. For example, the chaperone proteins have been demonstrated to facilitate the proper folding and solubility of heterologous proteins produced in bacterial host cells. Chen et al. J Bio Chem 274:19601-19605 (1999); U.S. Pat. Nos. 6,083,715; 6,027,888; Bothmann and Pluckthun, J. Biol. Chem. 275:17100-17105 (2000); Ramm and Pluckthun, J. Biol. Chem. 275:17106-17113 (2000); Arie et al., Mol. Microbiol. 39:199-210 (2001).

To minimize proteolysis of expressed heterologous proteins (especially those that are proteolytically sensitive), certain host strains deficient for proteolytic enzymes can be used for the present invention, as described in, for example, U.S. Pat. Nos. 5,264,365; 5,508,192; Hara et al., Microbial Drug Resistance, 2:63-72 (1996). E. coli strains deficient for proteolytic enzymes and transformed with plasmids overexpressing one or more chaperone proteins may be used as host cells in the expression system encoding the antibodies of the present application.

The antibodies produced herein can be further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75. Protein A immobilized on a solid phase for example can be used in some embodiments for immunoaffinity purification of binding molecules of the present disclosure. In some embodiments, the solid phase to which Protein A is immobilized is a column comprising a glass or silica surface. In some embodiments, the solid phase to which Protein A is immobilized is a controlled pore glass column or a silicic acid column. In some embodiments, the column has been coated with a reagent, such as glycerol, in an attempt to prevent nonspecific adherence of contaminants. The solid phase is then washed to remove contaminants non-specifically bound to the solid phase. Finally the antibodies of interest is recovered from the solid phase by elution.

### Recombinant Production in Eukarvotic Cells

For eukaryotic expression, the vector components generally include, but are not limited to, one or more of the following, a signal sequence, an origin of replication, one or more marker genes, and enhancer element, a promoter, and a transcription termination sequence.

A vector for use in a eukaryotic host may also an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available. The DNA for such precursor region can be ligated in reading frame to DNA encoding the antibodies of the present application.

Generally, the origin of replication component is not needed for mammalian expression vectors (the SV40 origin may typically be used only because it contains the early promoter).

Expression and cloning vectors may contain a selection gene, also termed a selectable marker. Selection genes may encode proteins that confer resistance to antibiotics or other toxins, e.g., ampicillin, neomycin, methotrexate, or tetracycline; complement auxotrophic deficiencies; or supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up nucleic acid encoding the antibodies of the present application. For example, cells transformed with the DHFR selection gene are first identified by culturing all of the transformants in a culture medium that contains methotrexate (Mtx), a competitive antagonist of DHFR. An exemplary appropriate host cell when wild-type DHFR is employed is the Chinese hamster ovary (CHO) cell line deficient in DHFR activity. Alternatively, host cells (particularly wild-type hosts that contain endogenous DHFR) transformed or co-transformed with the polypeptide encoding-DNA sequences, wild-type DHFR protein, and another selectable marker such as aminoglycoside 3′-phosphotransferase (APH) can be selected by cell growth in medium containing a selection agent for the selectable marker such as an aminoglycosidic antibiotic.

Expression and cloning vectors usually contain a promoter that is recognized by the host organism and is operably linked to the nucleic acid encoding the desired polypeptide sequences. Eukaryotic genes have an AT-rich region located approximately 25 to 30 based upstream from the site where transcription is initiated. Another sequence found 70 to 80 bases upstream from the start of the transcription of many genes may be included. The 3′ end of most eukaryotic may be the signal for addition of the poly A tail to the 3′ end of the coding sequence. All of these sequences may be inserted into eukaryotic expression vectors.

Polypeptide transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding the antibodies of the present disclosure by higher eukaryotes is often increased by inserting an enhancer sequence into the vector. Many enhancer sequences are now known from mammalian genes (globin, elastase, albumin, α-fetoprotein, and insulin). Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide encoding sequence, but is preferably located at a site 5′ from the promoter.

Expression vectors used in eukaryotic host cells (yeast, fungi, insect, plant, animal, human, or nucleated cells from other multicellular organisms) also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region.

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells can be transformed with the above-described expression or cloning vectors for antibodies production and cultured in conventional nutrient media modified as appropriate for inducing promoters, selecting transformants, or amplifying the genes encoding the desired sequences.

The host cells used to produce the antibodies of the present application may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibodies can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein composition prepared from the cells can be purified using, for example, hydroxylapatite chromatography, gel electrophoresis, dialysis, and affinity chromatography, with affinity chromatography being the preferred purification technique. The matrix to which the affinity ligand is attached is most often agarose, but other matrices are available. Mechanically stable matrices such as controlled pore glass or poly (styrene-divinyl) benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered. Following any preliminary purification step(s), the mixture comprising the antibody of interest and contaminants may be subjected to low pH hydrophobic interaction chromatography.

### Methods of Using the Antibodies

In one aspect, provided herein is a method of attenuating an activity of IL-23R on a cell, comprising exposing the cell to an effective amount of an antibody or antigen binding fragment thereof provided herein. In one aspect, provided herein is a method of attenuating an activity of IL-23R in a cell, comprising exposing the cell to an effective amount of an antibody or antigen binding fragment thereof provided herein.

In some embodiments, the antibody provided herein attenuates an IL-23R activity by at least about 10%. In some embodiments, the antibody provided herein attenuates an IL-23R activity by at least about 20%. In some embodiments, the antibody provided herein attenuates an IL-23R activity by at least about 30%. In some embodiments, the antibody provided herein attenuates an IL-23R activity by at least about 40%. In some embodiments, the antibody provided herein attenuates an IL-23R activity by at least about 50%. In some embodiments, the antibody provided herein attenuates an IL-23R activity by at least about 60%. In some embodiments, the antibody provided herein attenuates an IL-23R activity by at least about 70%. In some embodiments, the antibody provided herein attenuates an IL-23R activity by at least about 80%. In some embodiments, the antibody provided herein attenuates an IL-23R activity by at least about 90%. In some embodiments, the antibody provided herein attenuates an IL-23R activity by at least about 95%. In certain embodiments, the antibody described herein can attenuate (e.g., partially attenuate) an IL-23R activity by at least about 15% to about 65%. In certain embodiments, the antibody described herein can attenuate (e.g., partially attenuate) an IL-23R activity by at least about 20% to about 65%. In certain embodiments, the antibody described herein can attenuate (e.g., partially attenuate) an IL-23R activity by at least about 30% to about 65%.

A non-limiting example of an IL-23R activity is IL-23R mediated signaling. Thus, in certain embodiments, provided herein is a method of attenuating (e.g., partially attenuating) IL-23R mediated signaling in a cell, comprising exposing the cell to an effective amount of an antibody or antigen binding fragment thereof provided herein.

In some embodiments, the antibody provided herein attenuates IL-23R mediated signaling by at least about 10%. In some embodiments, the antibody provided herein attenuates IL-23R mediated signaling by at least about 20%. In some embodiments, the antibody provided herein attenuates IL-23R mediated signaling by at least about 30%. In some embodiments, the antibody provided herein attenuates IL-23R mediated signaling by at least about 40%. In some embodiments, the antibody provided herein attenuates IL-23R mediated signaling by at least about 50%. In some embodiments, the antibody provided herein attenuates IL-23R mediated signaling by at least about 60%. In some embodiments, the antibody provided herein attenuates IL-23R mediated signaling by at least about 70%. In some embodiments, the antibody provided herein attenuates IL-23R mediated signaling by at least about 80%. In some embodiments, the antibody provided herein attenuates IL-23R mediated signaling by at least about 90%. In some embodiments, the antibody provided herein attenuates IL-23R mediated signaling by at least about 95%. In certain embodiments, the antibody described herein can attenuate (e.g., partially attenuate) IL-23R mediated signaling by at least about 15% to about 65%. In certain embodiments, the antibody described herein can attenuate (e.g., partially attenuate) IL-23R mediated signaling by at least about 20% to about 65%. In certain embodiments, the antibody described herein can attenuate (e.g., partially attenuate) IL-23R mediated signaling by at least about 30% to about 65%.

In some embodiments, the antibodies provided herein attenuate IL-23R binding to at least one of its ligands.

Another non-limiting example of an IL-23R activity is binding to IL-23. Thus, in certain embodiments, provided herein is a method of attenuating (e.g., partially attenuating) the binding of IL-23R to IL-23, comprising exposing a cell to an effective amount of an antibody or antigen binding fragment thereof provided herein.

In some embodiments, the antibody provided herein attenuates the binding of IL-23R to IL-23 by at least about 10%. In some embodiments, the antibody provided herein attenuates the binding of IL-23R to IL-23 by at least about 20%. In some embodiments, the antibody provided herein attenuates the binding of IL-23R to IL-23 by at least about 30%. In some embodiments, the antibody provided herein attenuates the binding of IL-23R to IL-23 by at least about 40%. In some embodiments, the antibody provided herein attenuates the binding of IL-23R to IL-23 by at least about 50%. In some embodiments, the antibody provided herein attenuates the binding of IL-23R to IL-23 by at least about 60%. In some embodiments, the antibody provided herein attenuates the binding of IL-23R to IL-23 by at least about 70%. In some embodiments, the antibody provided herein attenuates the binding of IL-23R to IL-23 by at least about 80%. In some embodiments, the antibody provided herein attenuates the binding of IL-23R to IL-23 by at least about 90%. In some embodiments, the antibody provided herein attenuates the binding of IL-23R to IL-23 by at least about 95%. In certain embodiments, the antibody described herein can attenuate (e.g., partially attenuate) the binding of IL-23R to IL-23 by at least about 15% to about 65%. In certain embodiments, the antibody described herein can attenuate (e.g., partially attenuate) the binding of IL-23R to IL-23 by at least about 20% to about 65%. In certain embodiments, the antibody described herein can attenuate (e.g., partially attenuate) the binding of IL-23R to IL-23 by at least about 30% to about 65%.

Yet another non-limiting example of an IL-23R activity is signaling mediated by IL-23. Thus, in certain embodiments, provided herein is a method of attenuating (e.g., partially attenuating) IL-23 mediated signaling in a cell, comprising exposing the cell to an effective amount of an antibody or antigen binding fragment thereof provided herein.

In some embodiments, the antibody provided herein attenuates IL-23 mediated signaling by at least about 10%. In some embodiments, the antibody provided herein attenuates IL-23 mediated signaling by at least about 20%. In some embodiments, the antibody provided herein attenuates IL-23 mediated signaling by at least about 30%. In some embodiments, the antibody provided herein attenuates IL-23 mediated signaling by at least about 40%. In some embodiments, the antibody provided herein attenuates IL-23 mediated signaling by at least about 50%. In some embodiments, the antibody provided herein attenuates IL-23 mediated signaling by at least about 60%. In some embodiments, the antibody provided herein attenuates IL-23 mediated signaling by at least about 70%. In some embodiments, the antibody provided herein attenuates IL-23 mediated signaling by at least about 80%. In some embodiments, the antibody provided herein attenuates IL-23 mediated signaling by at least about 90%. In some embodiments, the antibody provided herein attenuates IL-23 mediated signaling by at least about 95%. In certain embodiments, the antibody described herein can attenuate (e.g., partially attenuate) IL-23 mediated signaling by at least about 15% to about 65%. In certain embodiments, the antibody described herein can attenuate (e.g., partially attenuate) IL-23 mediated signaling by at least about 20% to about 65%. In certain embodiments, the antibody described herein can attenuate (e.g., partially attenuate) IL-23 mediated signaling by at least about 30% to about 65%.

### Diagnostic Assays and Methods of Detection

In one aspect, the anti-IL-23R antibodies and fragments thereof of the present disclosure are useful for detecting the presence of IL-23R in a biological sample. In an aspect, the anti-IL-23R antibodies and fragments thereof of the present disclosure are useful for detecting the presence of IL-23R on the surface of a cell or cells in a biological sample. In another aspect, the anti-IL-23R antibodies and fragments thereof of the present disclosure are useful for detecting the amount of IL-23R in a biological sample. In an aspect, the anti-IL-23R antibodies and fragments thereof of the present disclosure are useful for detecting the amount of IL-23R on the surface of a cell or cells in a biological sample. Such anti-IL-23R antibodies may include those that bind to human IL-23R but do not otherwise alter IL-23R signaling activity. The term “detecting” or “detection,” as used herein, encompasses quantitative or qualitative detection, and refers to using readouts or results of an assay to make a determination with regard to the presence/absence of IL-23R in the sample, the relative expression level IL-23R in the sample (e.g. relative to one or more reference expressions, relative to other samples, or relative to one or more expression scales), or the concentration of IL-23R in the sample. The detected readout from the assay can be numerical data, e.g. results of immunoassays, or a measured protein concentration; the detected readout can be non-numerical data, e.g. cells or tissues staining with anti-IL-23R antibodies in microscopy images or staining of IL-23R in an imaging flow cytometry; or the detected readout can be any experimental data that can be used as a proxy for the IL-23R expression as is well known to a person skilled in the art, e.g. fluorescent intensity, luminescence intensity, colorimetric readings, and absorption/emission spectroscopy. The detected readout in the assays is referred to herein as the signal of the assay.

In some embodiments, anti-IL-23R antibodies and fragments thereof of the present disclosure bind to the native receptor on cells. In some embodiments, anti-IL-23R antibodies and fragments thereof of the present disclosure bind to and alter the native receptor on cells. In some embodiments, anti-IL-23R antibodies and fragments thereof of the present disclosure bind to the denatured receptor on Western blots. In some embodiments, anti-IL-23R antibodies and fragments thereof of the present disclosure can be used to immunoprecipitate the receptor from cell lysates. In some embodiments, anti-IL-23R antibodies and fragments thereof of the present disclosure can be used to study the cellular synthesis of the IL-23 receptor. In some embodiments, anti-IL-23R antibodies and fragments thereof of the present disclosure can be used to study the trafficking of the IL-23 receptor. In some embodiments, anti-IL-23R antibodies and fragments thereof of the present disclosure can be used to study the turnover of the IL-23 receptor. In some embodiments, anti-IL-23R antibodies and fragments thereof of the present disclosure can be used to study the mechanism of action for how therapeutics affect the cellular synthesis of the IL-23 receptor. In some embodiments, anti-IL-23R antibodies and fragments thereof of the present disclosure can be used to study the mechanism of action for how therapeutics affect the trafficking of the IL-23 receptor. In some embodiments, anti-IL-23R antibodies and fragments thereof of the present disclosure can be used to study the mechanism of action for how therapeutics affect the turnover of the IL-23 receptor. In some embodiments, anti-IL-23R antibodies and fragments thereof of the present disclosure can be used in predicting drug mechanism of action and efficacious doses/exposures. In some embodiments, anti-IL-23R antibodies and fragments thereof of the present disclosure can be used in predicting efficacious doses/exposures of therapeutics.

In some embodiments, anti-IL-23R antibodies and fragments thereof of the present disclosure can be used to detect the IL-23 receptor on human cells. In some embodiments, anti-IL-23R antibodies and fragments thereof of the present disclosure can be used to detect the IL-23 receptor on human IL-23R transfected cell lines. In some embodiments, anti-IL-23R antibodies and fragments thereof of the present disclosure can be used to detect the IL-23 receptor on human IL-23R transfected iLite cell lines. In some embodiments, anti-IL-23R antibodies and fragments thereof of the present disclosure can be used to detect the IL-23 receptor on human IL-23R transfected HEK cell lines. In some embodiments, anti-IL-23R antibodies and fragments thereof of the present disclosure can be used to detect the IL-23 receptor on primary human CD3⁺ CD56⁻ T cells. In some embodiments, anti-IL-23R antibodies and fragments thereof of the present disclosure can be used to detect the IL-23 receptor on primary human CD3⁺ CD56⁺ cells. In some embodiments, anti-IL-23R antibodies and fragments thereof of the present disclosure can be used to detect the IL-23 receptor on primary human CD3⁻ CD56⁺ cells.

In some embodiments, anti-IL-23R antibodies and fragments thereof of the present disclosure can be used to detect the IL-23 receptor on human cells to identify IL-23-responsive cells. In some embodiments, anti-IL-23R antibodies and fragments thereof of the present disclosure can be used to detect the IL-23 receptor on human cells to quantify IL-23-responsive cells. In some embodiments, anti-IL-23R antibodies and fragments thereof of the present disclosure can be used to detect the IL-23 receptor on human cells to predict which diseases will respond to IL-23 pathway targeted inhibitors. In some embodiments, anti-IL-23R antibodies and fragments thereof of the present disclosure can be used to detect the IL-23 receptor on human cells to predict which patient subsets will respond to IL-23 pathway targeted inhibitors.

In some embodiments, anti-IL-23R antibodies and fragments thereof of the present disclosure can be used to label IL-23R-expresssing cells in healthy subjects and disease. In some embodiments, anti-IL-23R antibodies and fragments thereof of the present disclosure can be used to sort IL-23R-expresssing cells in healthy subjects and disease. In some embodiments, anti-IL-23R antibodies and fragments thereof of the present disclosure can be used to isolate IL-23R-expresssing cells in healthy subjects and disease. In some embodiments, anti-IL-23R antibodies and fragments thereof of the present disclosure can be used to characterize IL-23R-expresssing cells in healthy subjects and disease. In some embodiments, anti-IL-23R antibodies and fragments thereof of the present disclosure can be used to characterize the underlying disease pathways. In some embodiments, anti-IL-23R antibodies and fragments thereof of the present disclosure can be used to understand mechanism of action of therapeutics. In some embodiments, anti-IL-23R antibodies and fragments thereof of the present disclosure can be used to identify markers of response to drugs.

In some embodiments, anti-IL-23R antibodies and fragments thereof of the present disclosure bind to the IL-23R in such a manner that they inhibit IL-23 binding and signaling. In some embodiments, anti-IL-23R antibodies and fragments thereof of the present disclosure bind to the IL-23R in such a manner that they compete for binding with IL-23R antagonist peptide therapeutics. In some embodiments, anti-IL-23R antibodies and fragments thereof of the present disclosure are for use in receptor occupancy target engagement assays. In some embodiments, anti-IL-23R antibodies and fragments thereof of the present disclosure are for use in receptor occupancy target engagement assays in blood and other tissues of patients dosed with IL-23R antagonist peptides. In some embodiments, anti-IL-23R antibodies and fragments thereof of the present disclosure are for use in receptor occupancy target engagement assays in blood and other tissues of patients dosed with other receptor-targeted therapeutics. In some embodiments, anti-IL-23R antibodies and fragments thereof of the present disclosure are used in target engagement assays to inform dose prediction in a range of different diseases. In some embodiments, anti-IL-23R antibodies and fragments thereof of the present disclosure are used in target engagement assays to inform selection for future therapeutics in a range of different diseases.

In some embodiments, anti-IL-23R antibodies and fragments thereof of the present disclosure that bind to different epitopes on IL-23R that can be used in different combinations to optimize detection of IL-23R-expressing cells. In some embodiments, anti-IL-23R antibodies and fragments thereof of the present disclosure that bind to different epitopes on IL-23R that can be used in different combinations to optimize staining of IL-23R-expressing cells. In some embodiments, anti-IL-23R antibodies and fragments thereof of the present disclosure that bind to different epitopes on IL-23R that can be used in different combinations to optimize visualization of IL-23R-expressing cells. In some embodiments, anti-IL-23R antibodies and fragments thereof of the present disclosure that bind to different epitopes on IL-23R that can be used in different combinations to optimize quantitation of IL-23R-expressing cells.

In particular embodiments, IL-23R antibodies and fragments thereof, particularly antigen-binding fragments, that bind to different epitopes on IL-23R, such as those that are set out in distinct bins, including as provided in Table 11, are used to optimize evaluation and/or quantitation of IL-23R or IL-23R-expressing cells. In an embodiment, combinations of an antibody with strong cell surface IL-23R binding and an antibody that binds to a distinct epitope, such as from a different epitope bin, that is a non-competing and also demonstrates strong cell surface IL-23R binding are provided and may be particularly preferred.

In embodiments hereof, combinations of a Bin 3 antibody and a non-Bin3 antibody are contemplated and may be utilized. In embodiments hereof, combinations of a Bin 1 antibody and a non-Bin1 antibody are contemplated and may be utilized. In embodiments hereof, combinations of a Bin 5 antibody and a non-Bin5 antibody are contemplated and may be utilized. In embodiments hereof, combinations of a Bin 2 antibody and a non-Bin2 antibody are contemplated and may be utilized. In embodiments hereof, combinations of a Bin 4 antibody and a non-Bin4 antibody are contemplated and may be utilized. In embodiments hereof, combinations of a Bin 6 antibody and a non-Bin6 antibody are contemplated and may be utilized. In embodiments hereof, combinations of a Bin 7 antibody and a non-Bin7 antibody are contemplated and may be utilized. In embodiments hereof, combinations of a Bin 8 antibody and a non-Bin8 antibody are contemplated and may be utilized. In embodiments hereof, combinations of a Bin 9 antibody and a non-Bin9 antibody are contemplated and may be utilized. In embodiments, combinations of a Bin2 and a Bin 1 antibody, or of a Bin 1 and Bin5 antibody, or of a Bin 1 and Bin4 antibody, or a Bin 2 and Bin 4 antibody, or a Bin 2 and a Bin3 antibody, or a Bin 1 and Bin5 antibody, or a Bin 2 and Bin5 antibody, or a Bin3 and Bin 5 antibody, or a Bin 4 and Bin 5 antibody, or a Bin 3 and Bin 7 antibody, or a Bin1 and Bin 7 antibody, or combinations of a Bin3 antibody and a Bin 1 antibody are comtemplated and may be utilized.

Bin 1 antibodies comprise antibody that binds to the same epitope and/or binds IL-23R competitively with the antibody comprising: a VH comprising the amino acid sequence of SEQ ID NO:202 or comprising the CDR1, CDR2 and CDR3 domain sequences thereof, and a VL comprising the amino acid sequence of SEQ ID NO:203 or comprising the CDR1, CDR2 and CDR3 domain sequences thereof; a VH comprising the amino acid sequence of SEQ ID NO:252 or comprising the CDR1, CDR2 and CDR3 domain sequences thereof, and a VL comprising the amino acid sequence of SEQ ID NO:253 or comprising the CDR1, CDR2 and CDR3 domain sequences thereof; or a VH comprising the amino acid sequence of SEQ ID NO:156 or comprising the CDR1, CDR2 and CDR3 domain sequences thereof, and a VL comprising the amino acid sequence of SEQ ID NO:157 or comprising the CDR1, CDR2 and CDR3 domain sequences thereof. Bin 2 antibodies comprise antibody that binds to the same epitope and/or binds IL-23R competitively with the antibody comprising: a VH comprising the amino acid sequence of SEQ ID NO:192 or comprising the CDR1, CDR2 and CDR3 domain sequences thereof, and a VL comprising the amino acid sequence of SEQ ID NO:193 or comprising the CDR1, CDR2 and CDR3 domain sequences thereof; a VH comprising the amino acid sequence of SEQ ID NO:194 or comprising the CDR1, CDR2 and CDR3 domain sequences thereof, and a VL comprising the amino acid sequence of SEQ ID NO:195 or comprising the CDR1, CDR2 and CDR3 domain sequences thereof; a VH comprising the amino acid sequence of SEQ ID NO:200 or comprising the CDR1, CDR2 and CDR3 domain sequences thereof, and a VL comprising the amino acid sequence of SEQ ID NO:201 or comprising the CDR1, CDR2 and CDR3 domain sequences thereof; a VH comprising the amino acid sequence of SEQ ID NO:218 or comprising the CDR1, CDR2 and CDR3 domain sequences thereof, and a VL comprising the amino acid sequence of SEQ ID NO:219 or comprising the CDR1, CDR2 and CDR3 domain sequences thereof; a VH comprising the amino acid sequence of SEQ ID NO:222 or comprising the CDR1, CDR2 and CDR3 domain sequences thereof, and a VL comprising the amino acid sequence of SEQ ID NO:223 or comprising the CDR1, CDR2 and CDR3 domain sequences thereof; a VH comprising the amino acid sequence of SEQ ID NO:260 or comprising the CDR1, CDR2 and CDR3 domain sequences thereof, and a VL comprising the amino acid sequence of SEQ ID NO:261 or comprising the CDR1, CDR2 and CDR3 domain sequences thereof; a VH comprising the amino acid sequence of SEQ ID NO:264 or comprising the CDR1, CDR2 and CDR3 domain sequences thereof, and a VL comprising the amino acid sequence of SEQ ID NO:265 or comprising the CDR1, CDR2 and CDR3 domain sequences thereof; a VH comprising the amino acid sequence of SEQ ID NO:154 or comprising the CDR1, CDR2 and CDR3 domain sequences thereof, and a VL comprising the amino acid sequence of SEQ ID NO: 155 or comprising the CDR1, CDR2 and CDR3 domain sequences thereof; a VH comprising the amino acid sequence of SEQ ID NO:160 or comprising the CDR1, CDR2 and CDR3 domain sequences thereof, and a VL comprising the amino acid sequence of SEQ ID NO:161 or comprising the CDR1, CDR2 and CDR3 domain sequences thereof; or a VH comprising the amino acid sequence of SEQ ID NO: 164 or comprising the CDR1, CDR2 and CDR3 domain sequences thereof, and a VL comprising the amino acid sequence of SEQ ID NO: 165 or comprising the CDR1, CDR2 and CDR3 domain sequences thereof. Bin 3 antibodies comprise antibody that binds to the same epitope and/or binds IL-23R competitively with the antibody comprising: a VH comprising the amino acid sequence of SEQ ID NO:786 or comprising the CDR1, CDR2 and CDR3 domain sequences thereof, and a VL comprising the amino acid sequence of SEQ ID NO:787 or comprising the CDR1, CDR2 and CDR3 domain sequences thereof; a VH comprising the amino acid sequence of SEQ ID NO:790 or comprising the CDR1, CDR2 and CDR3 domain sequences thereof, and a VL comprising the amino acid sequence of SEQ ID NO:791 or comprising the CDR1, CDR2 and CDR3 domain sequences thereof; a VH comprising the amino acid sequence of SEQ ID NO:792 or comprising the CDR1, CDR2 and CDR3 domain sequences thereof, and a VL comprising the amino acid sequence of SEQ ID NO:793 or comprising the CDR1, CDR2 and CDR3 domain sequences thereof; a VH comprising the amino acid sequence of SEQ ID NO:236 or comprising the CDR1, CDR2 and CDR3 domain sequences thereof, and a VL comprising the amino acid sequence of SEQ ID NO:237 or comprising the CDR1, CDR2 and CDR3 domain sequences thereof; a VH comprising the amino acid sequence of SEQ ID NO:826 or comprising the CDR1, CDR2 and CDR3 domain sequences thereof, and a VL comprising the amino acid sequence of SEQ ID NO:827 or comprising the CDR1, CDR2 and CDR3 domain sequences thereof; or a VH comprising the amino acid sequence of SEQ ID NO:828 or comprising the CDR1, CDR2 and CDR3 domain sequences thereof, and a VL comprising the amino acid sequence of SEQ ID NO:829 or comprising the CDR1, CDR2 and CDR3 domain sequences thereof. Bin 4 antibodies comprise antibody that binds to the same epitope and/or binds IL-23R competitively with the antibody comprising: a VH comprising the amino acid sequence of SEQ ID NO:196 or comprising the CDR1, CDR2 and CDR3 domain sequences thereof, and a VL comprising the amino acid sequence of SEQ ID NO:197 or comprising the CDR1, CDR2 and CDR3 domain sequences thereof; a VH comprising the amino acid sequence of SEQ ID NO:262 or comprising the CDR1, CDR2 and CDR3 domain sequences thereof, and a VL comprising the amino acid sequence of SEQ ID NO:263 or comprising the CDR1, CDR2 and CDR3 domain sequences thereof; or a VH comprising the amino acid sequence of SEQ ID NO:152 or comprising the CDR1, CDR2 and CDR3 domain sequences thereof, and a VL comprising the amino acid sequence of SEQ ID NO:153 or comprising the CDR1, CDR2 and CDR3 domain sequences thereof. Bin 5 antibodies comprise antibody that binds to the same epitope and/or binds IL-23R competitively with the antibody comprising: a VH comprising the amino acid sequence of SEQ ID NO:798 or comprising the CDR1, CDR2 and CDR3 domain sequences thereof, and a VL comprising the amino acid sequence of SEQ ID NO:799 or comprising the CDR1, CDR2 and CDR3 domain sequences thereof. Bin 6 antibodies comprise antibody that binds to the same epitope and/or binds IL-23R competitively with the antibody comprising: a VH comprising the amino acid sequence of SEQ ID NO:206 or comprising the CDR1, CDR2 and CDR3 domain sequences thereof, and a VL comprising the amino acid sequence of SEQ ID NO:207 or comprising the CDR1, CDR2 and CDR3 domain sequences thereof; a VH comprising the amino acid sequence of SEQ ID NO:250 or comprising the CDR1, CDR2 and CDR3 domain sequences thereof, and a VL comprising the amino acid sequence of SEQ ID NO:251 or comprising the CDR1, CDR2 and CDR3 domain sequences thereof; a VH comprising the amino acid sequence of SEQ ID NO:144 or comprising the CDR1, CDR2 and CDR3 domain sequences thereof, and a VL comprising the amino acid sequence of SEQ ID NO:145 or comprising the CDR1, CDR2 and CDR3 domain sequences thereof; or a VH comprising the amino acid sequence of SEQ ID NO: 170 or comprising the CDR1, CDR2 and CDR3 domain sequences thereof, and a VL comprising the amino acid sequence of SEQ ID NO: 171 or comprising the CDR1, CDR2 and CDR3 domain sequences thereof. Bin 7 antibodies comprise antibody that binds to the same epitope and/or binds IL-23R competitively with the antibody comprising: a VH comprising the amino acid sequence of SEQ ID NO:150 or comprising the CDR1, CDR2 and CDR3 domain sequences thereof, and a VL comprising the amino acid sequence of SEQ ID NO:151 or comprising the CDR1, CDR2 and CDR3 domain sequences thereof. Bin 8 antibodies comprise antibody that binds to the same epitope and/or binds IL-23R competitively with the antibody comprising: a VH comprising the amino acid sequence of SEQ ID NO:198 or comprising the CDR1, CDR2 and CDR3 domain sequences thereof, and a VL comprising the amino acid sequence of SEQ ID NO:199 or comprising the CDR1, CDR2 and CDR3 domain sequences thereof; or a VH comprising the amino acid sequence of SEQ ID NO:240 or comprising the CDR1, CDR2 and CDR3 domain sequences thereof, and a VL comprising the amino acid sequence of SEQ ID NO:241 or comprising the CDR1, CDR2 and CDR3 domain sequences thereof. Bin 9 antibodies comprise antibody that binds to the same epitope and/or binds IL-23R competitively with the antibody comprising: a VH comprising the amino acid sequence of SEQ ID NO:204 or comprising the CDR1, CDR2 and CDR3 domain sequences thereof, and a VL comprising the amino acid sequence of SEQ ID NO:205 or comprising the CDR1, CDR2 and CDR3 domain sequences thereof; or a VH comprising the amino acid sequence of SEQ ID NO:884 or comprising the CDR1, CDR2 and CDR3 domain sequences thereof, and a VL comprising the amino acid sequence of SEQ ID NO:885 or comprising the CDR1, CDR2 and CDR3 domain sequences thereof.

In embodiments, combinations of a Bin3 antibody and a Bin 1 antibody are comtemplated and may be utilized. In some embodiments one or more of antibody selected from I23RB1, I23RB3, I23RB4, I23RB76, I23RB269 or I23RB270, or an antibody comprising the heavy and light chain CDRs, or at least the heavy chain CDRs, of one or more of I23RB1, I23RB3, I23RB4, I23RB76, I23RB269 or I23RB270, may be combined with one or more antibody selected from I23RB42, I23RB85 or I23RB157, or an antibody comprising the heavy and light chain CDRs, or at least the heavy chain CDRs, of one or more of I23RB42, I23RB85 or I23RB157. In particular embodiments, antibody I23RB4 or I23RB76 or an antibody comprising the heavy and light chain CDRs, or at least the heavy chain CDRs, of I23RB4 or I23RB76 is combined with antibody I23RB42 or I23RB157 or an antibody comprising the heavy and light chain CDRs, or at least the heavy chain CDRs, of I23RB42 or I23RB157.

In some embodiments, the affinity of the anti-IL-23R antibodies and fragments thereof of the present disclosure for human IL-23R can be determined using Surface Plasmon Resonance (SPR) methods. In some embodiments, the affinity of the anti-IL-23R antibodies and fragments thereof of the present disclosure for human IL-23R can be determined using Biolayer Interferometry (BLI) methods. In some embodiments, the affinity of the anti-IL-23R antibodies and fragments thereof of the present disclosure for human IL-23R can be determined using flow cytometry.

In some embodiments, the present disclosure provides a method for detecting, selecting and/or enriching IL-23R in a sample comprising contacting the sample with the unlabeled or labeled anti-IL-23R antibodies. In certain embodiments, the method comprising using unlabeled or labeled anti-IL-23R antibodies in an IHC assay, an immunoblotting assay, a flow cytometry assay, an ELISA, a radioimmunoassay, or high throughput screening assay.

In some embodiments, the present disclosure provides a method for detecting IL-23R target engagement by another therapeutic targeting IL-23R, e.g. another antibody, a peptide or a small molecule, to support dose decisions and indication decisions based on how much target engagement is achieved.

In one aspect, the present disclosure provides a method of detecting the presence of IL-23R in a biological sample. In certain embodiments, the method comprises contacting the biological sample with an anti-IL-23R antibody under conditions permissive for binding of the anti-IL-23R antibody to IL-23R, and detecting the binding between an anti-IL-23R antibody and IL-23R.

As provided herein, a biological sample are samples or materials that have a biological origin. Biological samples include bodily fluid, a cell or a tissue sample. In some embodiments, the cell can be a cultured cell line, engineered cells, in vitro or ex vivo culture of cells obtained from a subject, e.g. a human subject, or cells obtained from a subject. In other embodiments, the tissue samples can include bodily fluid or a tissue obtained from a subject, including a human subject. In some embodiments, the tissue samples include samples from a region of a subject suspected of having a disease. Thus tissue samples can be, but are not limited to, tissues, bodily fluid, tissue fractions, and/or cells isolated from an organism such as a mammal, in particular a human. In a particular embodiment, the tissue samples are prepared into sections that have been formalin fixed, paraffin embedded (FFPE). The cells, bodily fluid, and/or the tissue can be obtained from a subject, e.g. a human subject, by methods well known to a person skilled in the art, e.g. biopsy (including liquid biopsy), surgical procedures, cell smear, and phlebotomy.

As is well known to a person skilled in the art, the amount of an antibody bound to an antigen in a biological sample correlates with the amount of the antigen in the biological sample. Therefore the amount of binding of an anti-IL-23R antibody as provided herein to IL-23R in a biological sample can be used to measure the amount or the expression level of IL-23R in the biological sample. In some embodiments, the amount of IL-23R in the biological sample is detected in the linear detection range of the anti-IL-23R antibody. The “linear detection range” refers to the range of the amount or concentration of an antigen, within which the bound antibody linearly correlates with the amount or concentration of the antigen in the biological sample. Such linear range depends on factors such as the affinity between the antibody and the antigen, the concentration of the antibody used for the antibody-antigen binding, and the condition used for the antibody-antigen binding.

The concentration of the antibody used for antibody-antigen binding can be assessed in a titration experiments. In one embodiment of such titration experiments, the concentration of an antibody is serially diluted by a factor of twofold, threefold, fivefold, or tenfold, and the serially diluted antibodies were each bound to control samples with known amount of the corresponding antigen. In one embodiment, the desirable antibody concentration is that at which the amount of antibody binding differentiates the broadest linear detection range.

The expression of IL-23R in a biological sample refers to the amount of IL-23R in the biological sample at a given time. The expression of IL-23R as measured reflects accumulation of IL-23R in the biological sample over a period of time and can include degradation or modification products such as full length IL-23R, fragments of IL-23R, and naturally modified, e.g. glycosylated, IL-23R. As proteins are translated from mRNAs, the levels of mRNA in the biological samples can be used as a proxy for the expression of IL-23R in the biological samples.

Provided herein are methods for assessing IL-23R expression in a tissue sample from a subject suspected of having an IL-23R-associated disease, including: (a) contacting said tissue sample with an antibody or antigen binding fragment thereof as provided herein; (b) detecting the binding of said antibody or antigen binding fragment thereof to said tissue sample; (c) determining the expression of IL-23R in the tissue sample, wherein the expression level of IL-23R in the tissue sample is compared with a reference expression level of IL-23R.

Also provided herein are methods for assessing IL-23R expression in a tissue sample from a subject suspected of having an IL-23R-associated disease, including: (a) performing an IHC assay on the tissue sample with an antibody or antigen binding fragment thereof as provided herein; (b) determining the expression of IL-23R in the tissue sample, wherein the expression level of IL-23R in the tissue sample is compared with a reference expression level of IL-23R.

Provided herein are also methods of assessing responsiveness of a patient to a therapeutic agent, e.g., IL-23 pathway targeted inhibitors, said method based on IL-23R expression in a tissue sample from said patient, including: (a) contacting said tissue sample with an antibody or antigen binding fragment thereof as provided herein; (b) detecting the binding of said antibody or antigen binding fragment thereof to said tissue sample; (c) determining the expression of IL-23R in the tissue sample, wherein the expression level of IL-23R in the tissue sample is compared with a reference expression level of IL-23R; wherein an increased expression level of IL-23R compared to the reference is indicative of responsiveness to said therapy.

Provided herein are also methods for assessing responsiveness of patient to a therapeutic agent, e.g., IL-23 pathway targeted inhibitors, said method based on IL-23R expression in a tissue sample from said patient, including: (a) performing an IHC assay on the tissue sample with an antibody or antigen binding fragment thereof as provided herein; (b) determining the expression of IL-23R in the tissue sample, wherein the expression level of IL-23R in the tissue sample is compared with a reference expression level of IL-23R, wherein an increased expression level of IL-23R in the tissue sample compared to the reference is indicative of responsiveness to said therapy.

Provided herein are methods of treating an IL-23R-associated disease in a subject including (a) determining expression level of IL-23R in a tissue sample from the subject with an antibody or antigen binding fragment thereof as provided herein, wherein the expression level of IL-23R in the tissue sample is higher than a reference expression level of IL-23R; (b) administering a therapeutic agent to the subject.

Provided herein are methods of treating an IL-23R-associated disease in a subject including (a) contacting a tissue sample from the subject with an antibody or antigen binding fragment thereof as provided herein; (b) determining expression level of IL-23R in the tissue sample from the subject with antibody or antigen binding fragment thereof as provided herein, wherein the expression level of IL-23R in the tissue sample is higher than a reference expression level of IL-23R; (c) administering a therapeutic agent to the subject.

Provided herein are methods of treating an IL-23R-associated disease in a subject including (a) obtaining a tissue sample from the subject; (b) contacting the tissue sample from the subject with an antibody or antigen binding fragment thereof as provided herein; (c) determining expression level of IL-23R in a tissue sample from the subject with an antibody or antigen binding fragment thereof as provided herein, wherein the expression level of IL-23R in the tissue sample is higher than a reference expression level of IL-23R; (d) administering a therapeutic agent to the subject.

The subject in any of the method provided herein can be a human subject and the subject can have any IL-23R-associated disease for which the determination of IL-23R expression can be performed and/or for which IL-23R expression can provide diagnostic, prognostic, or predictive value. Exemplary IL-23R-associated diseases include multiple sclerosis, asthma, rheumatoid arthritis, inflammatory bowel disease (IBD), juvenile IBD, adolescent IBD, Crohn's disease, sarcoidosis, systemic elitematodes, ankylosing spondylitis, Autoimmune inflammation such as inflammation (axial spondylitic arthritis), psoriatic arthritis, or psoriasis and related diseases and disorders. In certain embodiments, the disease or disorder is psoriasis (eg, psoriasis vulgaris, guttate psoriasis), inverse psoriasis, pustular psoriasis, palmo-plantar, Psoriasis, psoriasis vulgaris, or psoriatic psoriasis, atopic dermatitis, acne ectopica, ulcerative colitis, Crohn's disease, Celiac's disease Sexual sprue, enteropathy with seronegative arthritis, microscopic colitis, collagen-accumulating colitis, eosinophilia gastroenteritis/esophagitis, colitis associated with radiation or chemotherapy, leukocyte adhesion deficiency-1 Psoriasis with congenital immunity disorders, chronic granulomatosis, glycogenosis type 1b, Hermannsky-Padrac syndrome, Chediac-East syndrome, Wiscot-Aldrich syndrome, rectal colon resection and ileal anastomosis, Later ileal psoriasis, gastrointestinal cancer, pancreatitis, insulin-dependent diabetes, mammary psoriasis, psoriasis, bile ductitis, primary biliary cirrhosis, virus-related enteropathy, periductitis, chronic bronchitis, chronic sinusitis, asthma, Psoriasis, or implant-to-host disease. Exemplary IL-23R-associated diseases include multiple sclerosis, asthma, rheumatoid arthritis, inflammation of the gut, inflammatory bowel diseases (IBDs), juvenile IBD, adolescent IBD, Crohn's disease, ulcerative colitis, Celiac disease (nontropical Sprue), microscopic colitis, collagenous colitis, eosinophilic gastroenteritis/esophagitis, colitis associated with radio- or chemo-therapy, colitis associated with disorders of innate immunity as in leukocyte adhesion deficiency-1, sarcoidosis, Systemic Lupus Erythematosus, ankylosing spondylitis (axial spondyloarthritis), psoriatic arthritis, psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, enteropathy associated with seronegative arthropathies, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease. In some embodiments, the IL-23R-associated diseases are specifically selected from subset of inflammatory diseases, which include, but are not limited to psoriasis (PSO), psoriatic arthritis (PSA), inflammatory bowel disease (IBD), Crohn's disease (CD), or ulcerative colitis (UC). The disease or disorder may be an autoimmune disease. In some embodiments, the automimmine disease is selected from Ulcerative colitis (UC), Crohn's Disease (CD), psoriasis (PsO), or psoriatic arthritis (PsA).

As used herein, the term “responsiveness” refers to the probability of a cell or an individual or a patient or a subject responding or having a reaction to the treatment of a therapeutic agent. The responsiveness can include the probability of a wide range of reactions to the therapeutic agent, for example and without any limitation, (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy. In certain embodiments, the cell or tissue or individual or patient or subject is more likely to respond to a therapeutic agent, e.g., IL-23 pathway targeted inhibitors, when the cell, tissue, patient, or subject shows a significant expression IL-23R in its cells. In certain embodiments, the responsiveness increases with the increased level of IL-23R in cells. In certain embodiments, the responsiveness correlates with the level of IL-23R expression in cells. In certain embodiments, the responsiveness is linearly proportional to the level of IL-23R expression in cells. In other embodiments, the responsiveness of a cell, tissue, patient, or subject to a therapeutic agent can be 10%, 20%, 30%, 40%, 50%, 60%, 70%, 80%, 90%, 100%, 120%, 150%, 200%, 250%, 300%, 400%, 500%, 750%, 10 times, 15 times, 20 times, 30 times, 40 times, 50 times, 60 times, 70 times, 80 times, 90 times, 100 times, or more than the cells negative for IL-23R expression.

As used herein, “indicative of responsiveness” refers to a prediction that the responsiveness will be higher in one cell, tissue, human individual, or subject, over another cell, tissue, human individual, or subject. Such predictions are made based on certain criteria. In some embodiments, the prediction of the responsiveness is made based on the expression level of IL-23R in the cell, tissue, human individual, or subject. As described above, in certain embodiments, the cell or tissue or individual or patient or subject is predicted to be more likely to respond to a therapeutic agent, e.g., IL-23 pathway targeted inhibitors, when the cell, tissue, patient, or subject shows a significant expression of IL-23R in its cells. In certain embodiments, the responsiveness is predicted to increase with the increased level of IL-23R in its cells. In certain embodiments, the responsiveness is predicted to correlate with the level of IL-23R in its cells. In certain embodiments, the responsiveness is predicted to be linearly proportional to the level of IL-23R in its cells. In other embodiments, the responsiveness of a cell, tissue, patient, or subject to a therapeutic agent, e.g., IL-23 pathway targeted inhibitors, is predicted to be 10%, 20%, 30%, 40%, 50%, 60%, 70%, 80%, 90%, 100%, 120%, 150%, 200%, 250%, 300%, 400%, 500%, 750%, 10 times, 15 times, 20 times, 30 times, 40 times, 50 times, 60 times, 70 times, 80 times, 90 times, 100 times, or more than the cells negative for IL-23R expression.

The therapeutic agent can be any disease therapy for which IL-23R expression can provide diagnostic, prognostic, or predictive value. In certain embodiments, the therapeutic agent is one that targets biological processes, e.g. a signaling pathway, a metabolic pathway, or a protein synthesis/degradation pathway, in which IL-23R plays a role. In certain embodiments, the therapeutic agent is one that targets IL-23R activity, IL-23R signaling, or IL-23R mediated diseases. In a specific embodiment, the therapeutic agent is an anti-IL-23R antibody or an anti-IL-23R antibody drug conjugate.

For determining the expression of IL-23R in the tissue sample, the expression level of IL-23R in the tissue sample is compared or correlated with a reference expression level of IL-23R. The “reference expression level” as used herein refers to an expression level in a reference sample that, when compared with the IL-23R expression level in the test sample, provides relative information about IL-23R expression in the test sample. The reference expression level can be a known expression level, e.g. expression in a reference sample where the amount, concentration, and/or mole quantity of IL-23R is known. For example, the reference expression level can be the IL-23R expression in a cell line where the cells have been transfected or otherwise engineered to express a known amount of IL-23R. In some embodiments, reference expression level can be the IL-23R expression in a cell where the IL-23R expression has been quantitated by methods provided herein or known to a person skilled in the art, such as ELISA, SDS-PAGE, quantitative immunoprecipitation, and/or quantitative western blotting. In other embodiments, reference expression level involves more than one reference, such as 2, 3, 4, 5, 6, 7, 8, 9, 10, 12, 14, 16, 18, 20, 25, 30, 35, 40, 45, 50, 75, 100, and more references. In one embodiment, the reference level has 4 references: one negative for IL-23R expression, one with weak IL-23R expression, one with moderate IL-23R expression, and one with high/strong IL-23R expression.

Alternatively, a reference expression level can be a level where the exact concentration or quantity of IL-23R is unknown but the status, activity, and/or function of the reference sample is known. In some embodiments, the reference expression levels can be those of a non-diseased cell from the same patient that is suspected of having IL-23R-associated disease. In such embodiments, by comparing IL-23R expression in the test sample with that in the reference sample, the IL-23R expression in the test sample relative to (e.g. higher than, lower than, or substantially the same as) the IL-23R expression of the non-diseased cells can be determined. In other embodiments, the reference expression levels can be those of diseased cells, wherein by comparing IL-23R expression in the test sample with that in the reference sample, the IL-23R expression in the test sample relative to (e.g. higher than, lower than, or substantially the same as) the IL-23R expression of the diseased cells can be determined. In other embodiments, the reference expression levels can be those of a cell from a second subject, wherein by comparing IL-23R expression in the test sample with that in the reference sample, the IL-23R expression in the test sample relative to (e.g. higher than, lower than, or substantially the same as) the IL-23R expression of the cells from a second subject can be determined. The second subject can be a normal human subject having no IL-23R-associated disease, a human subject having the same kind of IL-23R-associated disease as that the patient is suspected of having, or a human subject having a different kind of IL-23R-associated disease from what the patient is suspected of having.

The reference sample and reference cells as described herein can be a cell line, in vitro or ex vivo culture of cells obtained from patient suspected of having IL-23R-associated disease, cells obtained from patient suspected of having IL-23R-associated disease, in vitro or ex vivo culture of cells obtained from a second subject, cells obtained from a second subject.

Therefore, in some embodiments of the methods provided herein, the reference expression level of IL-23R can be the IL-23R expression level in IL-23R-associated diseased cells, non-diseased cells of said subject, or non-diseased cells from a second subject.

As described above, in some embodiments, the IL-23R expression in a sample can be determined by correlating or comparing the anti-IL-23R antibody binding to the sample with the anti-IL-23R antibody binding to the negative and positive references, wherein the levels of IL-23R expression in the references are known. The reference IL-23R expression can be a positive IL-23R control or a negative IL-23R control. As used herein, a “positive IL-23R control” or a “positive control” refers to a cell, a tissue, a tumor, a human, and/or a subject known to express significant amount of IL-23R. A “negative IL-23R control” or “negative control” refers to cells and/or tissues known to express no IL-23R or low level of IL-23R such that the IL-23R in the cells or tissues are not biologically significant. IL-23R level is biologically insignificant when (1) a person skilled in the art would consider the level of IL-23R expression low in light of a protein product of a house keeping gene; (2) the presence or absence of the low amount of IL-23R does not make a biological difference to the cell or tissue; and (3) if these insignificant amount of IL-23R is removed or deleted from the cells or tissues, the cells or tissues will continue to function substantially the same way as before such removal or deletion. The known IL23R gene expression but not protein expression can be determined independently by a qPCR assay. The known IL-23R protein expression can also be independently determined using any below described immunoassays with an anti-IL-23R antibody that has been determined to be IL-23R specific, (e.g. an IL-23R specific antibody identified in the specificity-screening methods described above). Suitable immunoassays include, by way of example and without any limitation, an IHC assay, an immunoblotting assay, a FACS assay, or an ELISA. In some embodiments, the IL23R mRNA level may not be zero in the negative control due to the background noise of the assays or due to the biologically insignificant low level of remaining IL-23R. Negative IL-23R control can be properly considered negative for IL-23R, for example, when the IL23R mRNA level in a qPCR assay is substantially similar to the level of mRNA detected with a set of nonspecific primers, or when the IL23R mRNA level is biologically insignificant in light of the IL23R mRNA level in a positive IL-23R control. Similarly, the IL-23R detected by immunoassays using another IL-23R specific antibody in a negative control may not be zero due to background noise of the assays or due to the biologically insignificant low level of remaining IL-23R. The background noise can be caused by non-specific interactions between the assay reagents other than the anti-IL-23R antibodies and the samples. The negative IL-23R control can be properly considered negative for IL-23R, for example, when the IL-23R level in the negative control detected by the anti-IL-23R antibody is substantially similar to the detection level from an isotype control antibody in the same assay. In some assays, the background noise can account for a substantial percentage of the detected signal from the IL-23R expression.

Additionally, positive and negative controls have been identified and published in the literature by persons skilled in the art, including positive and/or negative tissues, cells (including, e.g. cell lines), and pathological samples. Such literature can be readily identified by searching databases such as PubMed (e.g. using search terms such as IL-23R, expression, positive, negative, and/or distribution) and analyzing the search hits.

Accordingly, the relative amount of the antibody or antigen-binding fragment thereof bound to a negative control cell with no IL-23R expression can be about 50%, 40%, 30%, 20%, 10%, 5%, 4%, 3%, 2%, 1%, 0.5%, 0.3%, 0.2%, 0.1%, 0.05%, 0.03%, 0.01%, 0.001% or less of the amount of the antibody or antigen-binding fragment thereof bound to the sample cell expressing IL-23R.

Therefore, in some embodiments of the methods provided herein, the reference expression level of IL-23R is the IL-23R expression level in a negative control, wherein the IL-23R expression in the negative control is independently determined by a qPCR assay, an IHC assay with a second antibody, an immunoblotting assay with a second antibody, a FACS assay with a second antibody, or an ELISA with a second antibody.

In other embodiments of the methods provided herein, the reference expression level of IL-23R is the IL-23R expression level in a positive control cell expressing IL-23R, wherein the IL-23R expression in the positive control is independently determined by a qPCR assay, an IHC assay with a second antibody, an immunoblotting assay with a second antibody, a FACS assay with a second antibody, or an ELISA with a second antibody.

The expression level of IL-23R in a sample can be different from that in a positive control. The relative amount of the antibody or antigen-binding fragment thereof bound to the tissue sample can be about 5%, 10%, 20%, 30%, 40%, 50%, 60%, 70%, 80%, 90%, 100%, 120%, 150%, 200%, 250%, 300%, 400%, 500%, 750%, 10 times, 15 times, 20 times, 30 times, 40 times, 50 times, 60 times, 70 times, 80 times, 90 times, 100 times, 200 times, 500 times, or more of the amount of the antibody or antigen-binding fragment thereof bound to the positive control cell expressing IL-23R.

Numerous different PCR or qPCR protocols are known in the art and exemplified herein below and can be directly applied or adapted for determining the IL23R mRNA level in the samples or controls, which can be used as a proxy for the expression levels of IL-23R protein in the samples or controls. Quantitative PCR (qPCR) (also referred as real-time PCR) is applied and adapted in some embodiments as it provides not only a quantitative measurement, but also reduced time and contamination. As used herein, “quantitative PCR (or “qPCR”) refers to the direct monitoring of the progress of PCR amplification as it is occurring without the need for repeated sampling of the reaction products. In quantitative PCR, the reaction products can be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time. When qPCR is applied to determine mRNA expression level, an extra step of reverse-transcription of mRNA to DNA is performed before the qPCR analysis. Examples of PCR methods can be found in the literature (Wong et al., BioTechniques 39:75-85 (2005); D'haene et al., Methods 50:262-270 (2010)), which is incorporated by reference herein in its entirety. Examples of PCR assays can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In one specific embodiment, qPCR can be performed to determine or measure the IL23R mRNA levels as follows. Briefly, mean Ct (cycle threshold) values (or referred to herein interchangeably as Cq (quantification cycle)) of replicate qPCR reactions for IL23R and one or more housekeeping genes are determined. Mean Ct values for IL23R can be then normalized to the Ct values of the housekeeping genes using the following exemplary formula: IL23R-ΔCt=(mean Ct of IL23R-mean Ct of housekeeping gene A). The relative IL23R-ΔCt can then be used to determine relative level of IL23R mRNA, for example by using the formula of mRNA expression=2^{-ΔCt}. For a summary of Ct and Cq values, see MIQE guideline (Bustin et al., The MIQE Guidelines: Minimum Information for Publication of Quantitative Real-Time PCR Experiments, Clinical Chemistry 55:4 (2009)).

Other commonly used methods known in the art for the quantification of mRNA expression in a sample can also be used, including northern blotting and in situ hybridization (Parker & Barnes, Methods in Molecular Biology 106:247-283 (1999)); RNAse protection assays (Hod, Biotechniques 13:852-854 (1992)); microarrays (Hoheisel et al., Nature Reviews Genetics 7:200-210 (2006); Jaluria et al., Microbial Cell Factories 6:4 (2007)); and polymerase chain reaction (PCR) (Weis et al, Trends in Genetics 8:263-264 (1992)). Alternatively, levels of mRNA expression can be determined by sequencing techniques. Representative methods for sequencing-based gene expression analysis include Serial Analysis of Gene Expression (SAGE), and gene expression analysis by massively parallel signature sequencing (MPSS).

As described earlier, tissue samples can be, but are not limited to, tissues, bodily fluid, tissue fractions, and/or cells isolated from an organism such as a mammal, in particular a human. Therefore, tissue samples can be obtained from a variety of organs of a subject, including a human subject. In some embodiments, tissue samples are obtained from organs suspected of having a disease, dysfunction or disorder, such as an immune disease. In other embodiments, tissue samples are obtained from normal organs from the patient who is being tested or from a second human subject.

Anti-IL-23R antibodies bound to the samples can be detected by a variety of immunoassays known in the art, including an IHC approach, an immunoblotting assay, a FACS assay, and an ELISA.

IL-23R can be detected by an anti-IL-23R antibody in a variety of IHC approaches. IHC staining of tissue sections has been shown to be a reliable method of assessing or detecting the presence of proteins in a sample. IHC techniques utilize an antibody to probe and visualize cellular antigens in situ, generally by chromogenic or fluorescent methods. Primary antibodies or antisera, such as polyclonal antisera and monoclonal antibodies that specifically target IL-23R, can be used to detect expression in an IHC assay. In some embodiments, the tissue sample is contacted with a primary antibody for a specific target for a period of time sufficient for the antibody-target binding to occur. As discussed in detail earlier, the antibodies can be detected by direct labels on the antibodies themselves, for example, radioactive labels, fluorescent labels, hapten labels such as biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. IHC protocols and kits are well known in the art and are commercially available. Automated systems for slide preparation and IHC processing are available commercially. The Leica BOND Autostainer and Leica Bond Refine Detection system is an example of such an automated system.

In some embodiments, an IHC assay is performed with an unlabeled primary antibody in conjunction with a labeled secondary antibody in an indirect assay. The indirect assay utilizes two antibodies for the detection of target proteins such as IL-23R in a tissue sample. First, an unconjugated primary antibody was applied to the tissue (first layer), which reacts with the target antigen in the tissue sample. Next, an enzyme-labeled secondary antibody is applied, which specifically recognizes the antibody isotype of the primary antibody (second layer). The secondary antibody reacts with the primary antibody, followed by substrate-chromogen application. The second-layer antibody can be labeled with an enzyme such as a peroxidase, which reacts with the chromogen 3, 3′-diaminobenzidine (DAB) to produce brown precipitate at the reaction site. This method is sensitive and versatile due to the potential signal amplification through a signal amplification system.

In certain embodiments to increase the sensitivity of the detection, a signal amplification system can be used. “A signal amplification system”, as used herein, means a system of reagents and methods that can be used to increase the signal from detecting the bound primary or the secondary antibody. A signal amplification system increases the sensitivity of the target protein detection, increases the detected signal, and decreases the lower boundary of the detection limits. There are several types of signal amplification systems including an enzyme labeling system and macrolabeling system. These systems/approaches are not mutually exclusive and can be used in combination for additive effect.

Macrolabels or macrolabeling system are collections of labels numbering in the tens (e.g. phycobiliproteins) to millions (e.g. fluorescent microspheres) attached to or incorporated in a common scaffold. The scaffold can be coupled to a target-specific affinity reagent such as an antibody, and the incorporated labels are thereby collectively associated with the target upon binding. The labels in the macrolabels can be any of the labels described herein such as fluorophores, haptens, enzymes, and/or radioisotopes. In one embodiment of the signal amplification system, a labeled chain polymer-conjugated secondary antibody was used. The polymer technology utilized an HRP enzyme-labeled inert “spine” molecule of dextran to which 1, 2, 3, 4, 5, 6, 7, 8, 9 10, 15, 20, 25, 30, 50 or more molecules of secondary antibodies can be attached, making the system even more sensitive.

Signal amplification system based on an enzyme labeling system utilizes the catalytic activity of enzymes, such as horseradish peroxidase (HRP) or alkaline phosphatase to generate high-density labeling of a target protein or nucleic acid sequence in situ. In one embodiment, tyramide can be used to increase the signal of HRP. In such a system, HRP enzymatically converts the labeled tyramide derivative into highly reactive, short-lived tyramide radicals. The labeled active tyramide radicals then covalently couple to residues (principally the phenol moiety of protein tyrosine residues) in the vicinity of the HRP-antibody-target interaction site, resulting in amplification of the number of labels at the site with minimal diffusion-related loss of signal localization. Consequently, the signal can be amplified 1, 2, 3, 4, 5, 6, 7, 8, 9 10, 15, 20, 25, 30, 50, 75, or 100 folds. As known to a person skilled in the art, the labels on the tyramide can be any labels described herein, including fluorophores, enzymes, haptens, radioisotopes, and/or photophores. Other enzyme-based reactions can be utilized to create signal amplification as well. For example, Enzyme-Labeled Fluorescence (ELF) signal amplification is available for alkaline phosphatase, wherein the alkaline phosphatase enzymatically cleaves a weakly blue-fluorescent substrate (ELF 97 phosphate) and converts it into a bright yellow-green-fluorescent precipitate that exhibits an unusually large Stokes shift and excellent photostability. Both tyramide-based signal amplification system and ELF signal amplification are available commercially, for example from ThermoFisher Scientific (Waltham, MA USA 02451).

Thus in some embodiments of the methods provided herein, the expression level of IL-23R is detected with a signal amplification system.

In some embodiments, the specimen is then counterstained to identify cellular and subcellular elements.

In some embodiments, the expression level of IL-23R can also be detected with antibodies described herein using an immunoblotting assay. In some embodiments of an immunoblotting assay proteins are often (but do not have to be) separated by electrophoresis and transferred onto membranes (usually nitrocellulose or PVDF membrane). Similar to the IHC assays, primary antibodies or antisera, such as polyclonal antisera and monoclonal antibodies that specifically target IL-23R, can be used to detect protein expression. In some embodiments, the membrane is contacted with a primary antibody for a specific target for a period of time sufficient for the antibody-antigen binding to occur and the bound antibodies can be detected by direct labels on the primary antibodies themselves, e.g. with radioactive labels, fluorescent labels, hapten labels such as biotin, or enzymes such as horseradish peroxidase or alkaline phosphatase. In other embodiments, unlabeled primary antibody is used in an indirect assay as described above in conjunction with a labeled secondary antibody specific for the primary antibody. As described herein, the secondary antibodies can be labeled, for example, with enzymes or other detectable labels such as fluorescent labels, luminescent labels, colorimetric labels, or radioisotopes. Immunoblotting protocols and kits are well known in the art and are commercially available. Automated systems for immunoblotting, e.g. iBind Western Systems for Western blotting (ThermoFisher, Waltham, MA USA 02451), are available commercially. Immunoblotting includes, but is not limited to, Western blot, in-cell Western blot, and dot blot. Dot blot is a simplified procedure in which protein samples are not separated by electrophoresis but are spotted directly onto a membrane. In cell Western blot involves seeding cells in microtiter plates, fixing/permeabilizing the cells, and subsequent detection with a primary labeled primary antibody or unlabeled primary antibody followed by labeled secondary antibody as described herein.

In other embodiments, the expression levels of IL-23R can also be detected with the antibodies described herein in a flow cytometry assay, including a fluorescence-activated cell sorting (FACS) assay. Similar to the IHC or immunoblotting assays, primary antibodies or antisera, such as polyclonal antisera and monoclonal antibodies that specifically target IL-23R, can be used to detect protein expression in a FACS assay. In some embodiments, cells are stained with primary antibodies against specific target protein for a period of time sufficient for the antibody-antigen binding to occur and the bound antibodies can be detected by direct labels on the primary antibodies, for example, fluorescent labels or hapten labels such as biotin on the primary antibodies. In other embodiments, unlabeled primary antibody is used in an indirect assay as described above in conjunction with a fluorescently labeled secondary antibody specific for the primary antibody. FACS provides a method for sorting or analyzing a mixture of fluorescently labeled biological cells, one cell at a time, based upon the specific light scattering and fluorescent characteristics of each cell. The flow cytometer thus detects and reports the intensity of the fluorchrome-tagged antibody, which indicates the expression level of the target protein. Therefore, the expression level of surface proteins (such as IL-23R) can be detected using antibodies against the target protein. Non-fluorescent cytoplasmic proteins can also be observed by staining permeablized cells. Methods for performing FACS staining and analyses are well known to a person skilled in the art and are described by Teresa S. Hawley and Robert G. Hawley in Flow Cytometry Protocols, Humana Press, 2011 (ISBN 1617379506, 9781617379505).

In other embodiments, the expression levels of IL-23R can also be detected using immunoassays such as an Enzyme Immune Assay (EIA) or an ELISA. Both EIA and ELISA assays are known in the art, e.g. for assaying a wide variety of tissues and samples, including blood, plasma, serum or bone marrow. A wide range of ELISA assay formats are available, see, e.g., U.S. Pat. Nos. 4,016,043, 4,424,279, and 4,018,653, which are hereby incorporated by reference in their entireties. These include both single-site and two-site or “sandwich” assays of the non-competitive types, as well as in the traditional competitive binding assays. These assays also include direct binding of a labeled antibody to a target protein. Sandwich assays are commonly used assays. A number of variations of the sandwich assay technique exist. For example, in a typical forward assay, an unlabeled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of target protein.

In some embodiments of the EIA or ELISA assays, an enzyme is conjugated to the second antibody. In other embodiments, fluorescently labeled secondary antibodies can be used in lieu of the enzyme-labeled secondary antibody to produce a detectable signal in ELISA assay format. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA and ELISA, the fluorescent labeled antibody is allowed to bind to the first antibody-target protein complex. After washing off the unbound reagent, the remaining tertiary complex is then exposed to the light of the appropriate wavelength, the fluorescence observed indicates the presence of the target protein of interest. Immunofluorescence and EIA techniques are both very well established in the art and are disclosed herein.

For the immunoassays described herein, any of a number of enzymes or non-enzyme labels can be utilized so long as the enzymatic activity or non-enzyme label, respectively, can be detected. The enzyme thereby produces a detectable signal, which can be utilized to detect a target protein. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, □-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of □- and □-galactosidase, □-glucuronidase,6-phospho-□-D-galatoside 6-phosphogalactohydrolase, □-gluosidase, □-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods of the present invention.

Various chromogenic or fluorogenic substrates to produce detectable signals are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), Chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-β-D-galactopyranoside and 2-Methoxy-4-(2-nitrovinyl)phenyl β-D-galactopyranoside for □-galactosidase; 2-Methoxy-4-(2-nitrovinyl)phenyl β-D-glucopyranoside for □-glucosidase; and the like. Exemplary fluorogenic substrates include, but are not limited to, 4-(Trifluoromethyl)umbelliferyl phosphate for alkaline phosphatase; 4-Methylumbelliferyl phosphate bis(2-amino-2-methyl-1,3-propanediol), 4-Methylumbelliferyl phosphate bis (cyclohexylammonium) and 4-Methylumbelliferyl phosphate for phosphatases; QuantaBlu™ and QuantaRed™ for horseradish peroxidase; 4-Methylumbelliferyl β-D-galactopyranoside, Fluorescein di(β-D-galactopyranoside) and Naphthofluorescein di-(β-D-galactopyranoside) for β-galactosidase; 3-Acetylumbelliferyl β-D-glucopyranoside and 4-Methylumbelliferyl-β-D-glucopyranoside for β-glucosidase; and 4-Methylumbelliferyl-α-D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are well known and commercially available (Pierce, Rockford IL; Santa Cruz Biotechnology, Dallas TX; Invitrogen, Carlsbad CA; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

In some embodiments of the immunoassays, a detectable label can be directly coupled to either the primary antibody or the secondary antibody that detects the unlabeled primary antibody can have. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene Oreg.); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway NJ), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

Methods well known to a person skilled in the art such as microscopy or spectroscopy can be utilized to visualize chromogenic or fluorescent detectable signals associated with the bound primary or secondary antibodies.

“Determining the expression of IL-23R” refers to putting the expression of IL-23R in the test biological sample in a referenced system (or a referenced scale) so that a person familiar with the referenced system can tell the relative expression of IL-23R in the test sample with respect to other samples that have been positioned in the referenced system. Consequently, such a determination involves comparing the expression of IL-23R in the test tissue sample with a reference expression level of IL-23R.

In some embodiments, the referenced system can be a quantitative system where the actual molar concentration or mole quantity of IL-23R in the test sample is compared with the molar concentration or mole quantity of the reference expression levels. In some embodiments, other surrogate numerical measurements that are linearly proportional to the molar concentration or mole quantity can be used. Examples of these surrogate measurements include fluorescence intensity measurements of bound antibodies, luminescence intensity measurements of bound antibodies, radioactivity measurements of bound antibodies, and/or colorimetric or chromogenic measurements of bound antibodies.

In other embodiments, the referenced system can be a categorization system. A categorization system can have as few as 2 categories and as many as 3, 4, 5, 6, 7, 8, 9, 10, 12, 14, 16, 18, 20, 25, 30, 35, 40, 45, 50, 75, 100, or more categories. In one embodiment, the referenced system has a positive category for samples that are positive for IL-23R expression (positive) and a negative category for samples that are negative for IL-23R expression (negative). In such a categorization system, determining the expression of IL-23R in a test sample involves comparing the expression of IL-23R in the test sample with IL-23R expression of a positive reference sample and/or with a negative reference, and placing the test sample in the positive category if the test sample expresses IL-23R at a similar or higher level than the positive reference. In another embodiment, the categorization system has 3 categories, e.g. negative, medium, and high expression categories. In another embodiment, the categorization system has 4 categories, e.g. negative, low/weak, medium/moderate, and high/strong expression categories. In some aspects, determining the expression of IL-23R in a test sample involves comparing the expression of IL-23R in the test sample with one or more references of IL-23R expression of known categories and placing the test sample in a category according to its relative expression to the reference(s).

In some embodiments, the referenced system can be a scoring system. A scoring system can be similar to a categorization system except that the categories are replaced by a score. For example, a 2-category system of negative and positive IL-23R expression can be a scoring system of 0 and 1, where 0 means negative and 1 means positive. In another embodiment, a 3-category system can be a scoring system of 0, 1, and 2, wherein 0 is negative, 1 is low/weak expression, and 2 is medium/moderate and high/strong expression. In another embodiment, a 4-category system can be a scoring system of 0, 1, 2, and 3, where in 0 is negative, 1 is low/weak, 2 is medium/moderate, and 3 is high/strong expression. Similar to the categorization system, determining the expression of IL-23R in a test sample in the scoring system involves comparing the expression of IL-23R in the test sample with one or more references of IL-23R expression having a known score and assigning the test sample a score according to its relative expression to the references. The scoring system, however, can be scores of discontinuous and discrete numbers (e.g. a system of 0, 2, 4, 6, 8, and 10, or a system of 1, 4, 6, 11, 13, and 19), numbers with fractions (e.g. 1, 1.5, 2, 2.5, 3, 3.5 etc), negative numbers, numbers starting from any number (e.g. 1000, 2000, 3000, etc), and any set of numbers that can be used to track the relative protein expression in biological samples.

In some embodiments, the referenced system can be percentages of cells in conjunction with the categorization or scoring systems. The combination of percentage of cells with a categorization or scoring system provides finer gradation of the samples. In such a combined system, each cell from the biological sample is assigned or placed in the categorization or scoring system, and the approximate percentage of cells of the test sample in each category or score is determined. In one example, measurements of percentages of cells can be coupled with a 2-category system of positive and negative IL-23R expression, wherein the sample is not measured as a whole but break into the percentage of cells that are positive for IL-23R expression and percentage of cells that are negative for IL-23R expression. For example, in a 2-category system of positive and negative IL-23R expression, a test sample may be negative for IL-23R expression if placed in a 2 category system but have 10% cells positive for IL-23R expression and 90% of cells negative for IL-23R expression. In one embodiment, the referenced system includes percentages of cells in conjunction with a 2-category or 2-score system. In one embodiment, the referenced system includes percentages of cells in conjunction with a 3-category or 3-score system. In one embodiment, the referenced system includes percentages of cells in conjunction with a 4-category or 4-score system. In one embodiment, the referenced system includes percentages of cells in conjunction with a 5-category or 5-score system. In one embodiment, the referenced system includes percentages of cells in conjunction with a 6-category or 6-score system. In one embodiment, the referenced system includes percentages of cells in conjunction with a 7-category or 7-score system. In one embodiment, the referenced system includes percentages of cells in conjunction with an 8-category or 8-score system. In one embodiment, the referenced system includes percentages of cells in conjunction with a 9-category or 9-score system. In one embodiment, the referenced system includes percentages of cells in conjunction with a 10-category or 10-score system. In one embodiment, the referenced system includes percentages of cells in conjunction with any categorization or any scoring system. In one embodiment, the referenced system includes percentages of cells in conjunction with a 4-score system comprising 0 (negative for IL-23R), 1 (low/weak IL-23R expression), 2 (medium/moderate IL-23R expression), and 3 (high/strong IL-23R expression).

In some aspects, the referenced system can be a ratio of the IL-23R expression to the expression of at least one reference protein. In some embodiments, the ratio could be a ratio of quantitative measurements of IL-23R expression and those of a reference protein, wherein the quantitative measurements include those described herein such as molar concentration, mole quantity, fluorescence intensity measurements of bound antibodies, luminescence intensity measurements of bound antibodies, radioactivity measurements of bound antibodies, and/or colorimetric or chromogenic measurements of bound antibodies. In other embodiments, the ratio could also be a ratio of scores assigned to IL-23R expression and the reference protein according to any scoring system provided herein. A reference protein could be a protein of ordinary cell function such as actin, GAPDH, GDI, or any protein expressed by a house-keeping gene. For example, if a test sample has a score of 3 for IL-23R expression and score of 1 for expression of actin, the ratio can be determined to be 3.

In other aspects, the reference system can utilize the results of a mathematical function using one or more of the categorization, score, ratio, percentage, and quantitative measurement as input. In some embodiments, the mathematical function uses compounding, addition, multiplication, or combination these operators to combine the various inputs to produce results that provide more detailed information about IL-23R expression in the sample and/or finer gradation on the IL-23R expression scale. In one embodiment, the mathematical function calculates an H score based on the combination of percentage and a 4-score system. For example, an H-score was calculated by summing the products of the percentage of cells (0-100) having each IL-23R expression score (0=negative, 1=low/weak, 2=moderate/medium, and 3=high/strong). For example: a specimen with 10% of cells scoring 3, 30% of cells scoring 2, 20% of cells scoring 1, and 40% of cells scoring 0 would have an H-score of (3×10)+(2×30)+(1×20)+(0×40)=110.

Therefore, as provided herein, the expression level of IL-23R can be determined using a categorization system, a scoring system, a ratio of IL-23R expression to the expression of at least one reference protein, percentage of cells in said categorization or scoring system, a quantitative measurement of IL-23R staining signal, or a result of a mathematical function using one or more of said categorization, score, ratio, percentage, and quantitative measurement as input.

Most formalin-fixed tissues require an antigen retrieval step before immunohistochemical staining. Methylene bridges formed during fixation cross-link proteins and mask the epitopes of the antigens. Antigen retrieval methods break these methylene bridges and expose the epitopes, allowing antibodies to bind. In some embodiments, antigens are retrieved by a heat induced epitope retrieval (HIER) method. In other embodiments, antigens are retrieved by an enzymatic retrieval (e.g. proteolytic digestion) method. For HIER, formalin-fixed, paraffin-embedded (FFPE) tissue sections can be heated at 50° C., 55° C., 60° C., 65° C., 70° C., 75° C., 80° C., 85° C., 90° C., 95° C., 100° C., 105° C., 110° C., 115° C., 120° C., 125° C., or 130° C. The FFPE tissue section can be heated at any of these temperatures for 5, 10, 15, 20, 25, 30, 35, 40, 45, 50, 55, 60, 65, 70, 75, 80, 85, 90, 95, 100, 105, 110, 115, 120, 150, 180, 240, 300, 360, 420, 480, 540, 600, 660, 720, 780, 840, 900, 960, 1020, 1080, 1140, 1200, 1260, 1320, 1380, and 1440 minutes. The HIER procedure at any of the aforementioned temperatures and for any of the aforementioned durations can be performed in a solution having a pH of 2, 2.5, 3, 3.5, 4, 4.5, 5, 5.2, 5.4, 5.6, 5.8, 6.0, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, 10, 10.2, 10.4, 10.6, 10.8, 11, 11.5, or 12. In some embodiments, the HIER can be performed at a temperature selected from a group consisting of 80° C., 85° C., 90° C., 95° C., 100° C., 105° C., 110° C., 115° C., for a duration selected from a group consisting of 5, 10, 15, 20, 25, 30, 35, 40, and at a pH selected from a group consisting of 8, 8.2, 8.4, 8.6, 8.8, 9, 9.2, 9.4, 9.6, 9.8, 10, 10.2, 10.4.

Thus, the methods provided herein further include a step of retrieving the epitope of IL-23R by heat-induced epitope retrieval (HIER).

Unlabeled antibodies, labeled antibodies and derivatives and analogs thereof, which immunospecifically bind to an IL-23R antigen can be used for diagnostic purposes to detect, diagnose, or monitor an IL-23R-mediated disease. Thus, provided herein are methods for the detection of an IL-23R-mediated disease comprising: (a) assaying the expression of an IL-23R antigen in cells or a tissue sample of a subject using one or more antibodies provided herein that immunospecifically bind to the IL-23R antigen; and (b) comparing the level of the IL-23R antigen with a control level, e.g., levels in normal tissue samples (e.g., from a patient not having an IL-23R-mediated disease, or from the same patient before disease onset), whereby an increase in the assayed level of IL-23R antigen compared to the control level of the IL-23R antigen is indicative of an IL-23R-mediated disease.

Also provided herein is a diagnostic assay for diagnosing an IL-23R-mediated disease comprising: (a) assaying for the level of an IL-23R antigen in cells or a tissue sample of an individual using one or more antibodies provided herein that immunospecifically bind to an IL-23R antigen; and (b) comparing the level of the IL-23R antigen with a control level, e.g., levels in normal tissue samples, whereby an increase in the assayed IL-23R antigen level compared to the control level of the IL-23R antigen is indicative of an IL-23R-mediated disease. In certain embodiments, provided herein is a method of treating an IL-23R-mediated disease in a subject, comprising: (a) assaying for the level of an IL-23R antigen in cells or a tissue sample of the subject using one or more antibodies provided herein that immunospecifically bind to an IL-23R antigen; and (b) comparing the level of the IL-23R antigen with a control level, e.g., levels in normal tissue samples, whereby an increase in the assayed IL-23R antigen level compared to the control level of the IL-23R antigen is indicative of an IL-23R-mediated disease. In some embodiments, the method further comprises (c) administering an effective amount of an antibody provided herein to the subject identified as having the IL-23R-mediated disease. A more definitive diagnosis of an IL-23R-mediated disease may allow health professionals to employ preventative measures or aggressive treatment earlier thereby preventing the development or further progression of the IL-23R-mediated disease.

Antibodies provided herein can be used to assay IL-23R antigen levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (1251, 1211), carbon (14C), sulfur (35S), tritium (3H), indium (121In), and technetium (99Tc); luminescent labels, such as luminol; and fluorescent labels, such as fluorescein and rhodamine, and biotin.

One aspect provided herein is the detection and diagnosis of an IL-23R-mediated disease in a human. In one embodiment, diagnosis comprises: a) administering (for example, parenterally, subcutaneously, or intraperitoneally) to a subject an effective amount of a labeled antibody that immunospecifically binds to an IL-23R antigen; b) waiting for a time interval following the administering for permitting the labeled antibody to concentrate at sites in the subject where the IL-23R antigen is expressed (and for unbound labeled molecule to be cleared to background level); c) determining background level; and d) detecting the labeled antibody in the subject, such that detection of labeled antibody above the background level indicates that the subject has an IL-23R-mediated disease. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99Tc. The labeled antibody will then accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled antibody to concentrate at sites in the subject and for unbound labeled antibody to be cleared to background level is 6 to 48 hours or 6 to 24 hours or 6 to 12 hours. In another embodiment, the time interval following administration is 5 to 20 days or 5 to 10 days.

In one embodiment, monitoring of an IL-23R-mediated disease is carried out by repeating the method for diagnosing the IL-23R-mediated disease, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods provided herein include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### Methods of Therapeutic Use

In another aspect, provided herein is a method of treating a disease or disorder in a subject comprising administering to the subject an effective amount of an antibody or antigen binding fragment thereof provided herein. In one embodiment, the disease or disorder is an IL-23R-mediated disease or disorder. In one embodiment, the disease or disorder is an IL-23-associated disease or disorder. Also provided herein is a method of treatment of a disease or disorder, wherein the subject is administered one or more therapeutic agents in combination with the antibody or antigen-binding fragment thereof provided herein.

The disclosure also relates to methods of using the antibodies provided herein to inhibit, i.e. antagonize, function of IL-23R in order to inhibit STAT activation. In some embodiments, the antibodies provided herein inhibit function of IL-23R in order to inhibit STAT3 activation. In some embodiments, the antibodies provided herein inhibit STAT3 phosphorylation.

The disclosure also to a method of treating or preventing an IL-23R associated disease or disorder in a subject in need thereof, comprising administering to the subject an isolated monoclonal antibody or antigen binding fragment thereof that specifically binds IL-23R or a pharmaceutical composition of the invention. The disclosure also relates to a method of treating or preventing an autoimmune disease in a subject in need thereof, comprising administering to the subject an isolated monoclonal antibody or antigen binding fragment thereof that specifically binds IL-23R or a pharmaceutical composition of the invention. In some embodiments, the autoimmune disease is selected from subset of inflammatory diseases, which include, but are not limited to psoriasis (PSO), psoriatic arthritis (PSA), inflammatory bowel disease (IBD), Crohn's disease (CD), or ulcerative colitis (UC).

The pharmaceutical composition comprises a therapeutically effective amount of an anti-IL-23R antibody or antigen binding fragment thereof. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to anti-IL-23R antibodies or antigen-binding fragments thereof, a therapeutically effective amount means an amount of the anti-IL-23R antibody or antigen-binding fragment thereof that modulates an immune response in a subject in need thereof.

A therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to an autoimmune disease or disorder (e.g., psoriasis), which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as an autoimmune disorder (e.g., psoriasis). In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are compositions used in the treatment or prevention of an autoimmune disease. For an autoimmune therapy, the compositions can be used in combination with another treatment including, but not limited to, a chemotherapy, an anti-HLA-Cw6 mAb, anti-IL-23 mAb, an anti-TNF-alpha mAb, an anti-IL-17A mAb, other autoimmune disease drugs, an antibody-drug conjugate (ADC), or a targeted therapy. Anti-IL-23R antibodies can be used to construct bispecific antibodies with partner mAbs against HLA-Cw6, TNF-alpha, IL-17A, and/or other cell surface antigens to treat autoimmune diseases that express both IL-23R and the specific cell surface antigen.

As used herein, the term “in combination,” in the context of the administration of two or more distinct therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

As used herein, in aspects the term “in combination,” in the context of the administration of two or more distinct antibodies particularly including two or more distinct IL-23R antibodies or a first IL-23R antibody and a second antibody which may bind IL-23R or which may not bind IL-23R, to a subject, refers to the use of more than one antibody together. The use of the term “in combination” does not restrict the order in which the antibodies are provided in a particular context or administered to a subject. For example, a first antibody (e.g., a composition comprising an IL-23R antibody described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second antibody to a subject.

Methods of administration and dosing is described in more detail in below.

In another aspect, provided herein is the use of the antibody or antigen binding fragment thereof provided herein in the manufacture of a medicament for treating a disease or disorder in a subject.

In another aspect, provided herein is the use of a pharmaceutical composition provided herein in the manufacture of a medicament for treating a disease or disorder in a subject.

In another aspect, provided herein is the use of an antibody or antigen binding fragment thereof provided herein in the manufacture of a medicament, wherein the medicament is for use in a method for detecting the presence of an IL-23R in a biological sample, the method comprising contacting the biological sample with the antibody under conditions permissive for binding of the antibody to the IL-23R protein, and detecting whether a complex is formed between the antibody and the IL-23R protein.

In a specific embodiment, provided herein is a composition for use in the prevention and/or treatment of a disease or condition comprising an antibody or antigen binding fragment thereof provided herein. In one embodiment, provided herein is a composition for use in the prevention of a disease or condition, wherein the composition comprises an antibody or antigen binding fragment thereof provided herein. In one embodiment, provided herein is a composition for use in the treatment of a disease or condition, wherein the composition comprises an antibody or antigen binding fragment thereof provided herein. In some embodiments, the disease or condition is an IL-23R-mediated disease. In some embodiments, the disease or condition is an IL-23-mediated disease. In some embodiments, the disease or disorder is associated with IL-23R. In some embodiments, the disease or disorder is an autoimmune disease. In certain embodiments, the disease or disorder is multiple sclerosis, asthma, rheumatoid arthritis, inflammatory bowel disease (IBD), juvenile IBD, adolescent IBD, Crohn's disease, sarcoidosis, systemic elitematodes, ankylosing spondylitis, Autoimmune inflammation such as inflammation (axial spondylitic arthritis), psoriatic arthritis, or psoriasis and related diseases and disorders. In certain embodiments, the disease or disorder is psoriasis (eg, psoriasis vulgaris, guttate psoriasis), inverse psoriasis, pustular psoriasis, palmo-plantar, Psoriasis, psoriasis vulgaris, or psoriatic psoriasis, atopic dermatitis, acne ectopica, ulcerative colitis, Crohn's disease, Celiac's disease Sexual sprue, enteropathy with seronegative arthritis, microscopic colitis, collagen-accumulating colitis, eosinophilia gastroenteritis/esophagitis, colitis associated with radiation or chemotherapy, leukocyte adhesion deficiency-1 Psoriasis with congenital immunity disorders, chronic granulomatosis, glycogenosis type 1b, Hermannsky-Padrac syndrome, Chediac-East syndrome, Wiscot-Aldrich syndrome, rectal colon resection and ileal anastomosis, Later ileal psoriasis, gastrointestinal cancer, pancreatitis, insulin-dependent diabetes, mammary psoriasis, psoriasis, bile ductitis, primary biliary cirrhosis, virus-related enteropathy, periductitis, chronic bronchitis, chronic sinusitis, asthma, Psoriasis, or implant-to-host disease. In some embodiments, the disease or disorder is selected from subset of inflammatory diseases, which include, but are not limited to psoriasis (PSO), psoriatic arthritis (PSA), inflammatory bowel disease (IBD), Crohn's disease (CD), or ulcerative colitis (UC).

In certain embodiments, the subject is a subject in need thereof. In some embodiments, the subject has the disease or condition. In other embodiments, the subject is at risk of having the disease or condition. In some embodiments, the administration results in the prevention, management, treatment or amelioration of the disease or condition.

In one embodiment, provided herein is a composition for use in the prevention and/or treatment of a symptom of a disease or condition, wherein the composition comprises an antibody or antigen binding fragment thereof provided herein. In one embodiment, provided herein is a composition for use in the prevention of a symptom of a disease or condition, wherein the composition comprises an antibody or antigen binding fragment thereof provided herein. In one embodiment, provided herein is a composition for use in the treatment of a symptom of a disease or condition, wherein the composition comprises an antibody or antigen binding fragment thereof provided herein. In some embodiments, the disease or condition is an IL-23R-mediated and/or IL-23 mediated disease. In some embodiments, the disease or disorder is associated with IL-23R. In some embodiments, the disease or disorder is an autoimmune disease. In certain embodiments, the disease or disorder is multiple sclerosis, asthma, rheumatoid arthritis, inflammatory bowel disease (IBD), juvenile IBD, adolescent IBD, Crohn's disease, sarcoidosis, systemic elitematodes, ankylosing spondylitis, Autoimmune inflammation such as inflammation (axial spondylotic arthritis), psoriatic arthritis, or psoriasis and related diseases and disorders. In certain embodiments, the disease or disorder is psoriasis (eg, psoriasis vulgaris, guttate psoriasis), inverse psoriasis, pustular psoriasis, palmo-plantar, Psoriasis, psoriasis vulgaris, or psoriatic psoriasis, atopic dermatitis, acne ectopica, ulcerative colitis, Crohn's disease, Celiac's disease Sexual sprue, enteropathy with seronegative arthritis, microscopic colitis, collagen-accumulating colitis, eosinophilia gastroenteritis/esophagitis, colitis associated with radiation or chemotherapy, leukocyte adhesion deficiency-1 Psoriasis with congenital immunity disorders, chronic granulomatosis, glycogenosis type 1b, Hermannsky-Padrac syndrome, Chediac-East syndrome, Wiscot-Aldrich syndrome, rectal colon resection and ileal anastomosis, Later ileal psoriasis, gastrointestinal cancer, pancreatitis, insulin-dependent diabetes, mammary psoriasis, psoriasis, bile ductitis, primary biliary cirrhosis, virus-related enteropathy, periductitis, chronic bronchitis, chronic sinusitis, asthma, Psoriasis, or implant-to-host disease. In some embodiments, the disease or disorder is selected from subset of inflammatory diseases, which include, but are not limited to psoriasis (PSO), psoriatic arthritis (PSA), inflammatory bowel disease (IBD), Crohn's disease (CD), or ulcerative colitis (UC). In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

In another embodiment, provided herein is a method of preventing and/or treating a disease or condition in a subject, comprising administering an effective amount of an antibody or antigen binding fragment thereof provided herein. In one embodiment, provided herein is a method of preventing a disease or condition in a subject, comprising administering an effective amount of an antibody or antigen binding fragment thereof provided herein. In one embodiment, provided herein is a method of treating a disease or condition in a subject, comprising administering an effective amount of an antibody or antigen binding fragment thereof provided herein. In some embodiments, the disease or condition is an IL-23R-mediated and/or IL-23 mediated disease. In some embodiments, the disease or disorder is associated with IL-23R. In some embodiments, the disease or disorder is an autoimmune disease. In certain embodiments, the disease or disorder is multiple sclerosis, asthma, rheumatoid arthritis, inflammatory bowel disease (IBD), juvenile IBD, adolescent IBD, Crohn's disease, sarcoidosis, systemic elitematodes, ankylosing spondylitis, Autoimmune inflammation such as inflammation (axial spondylotic arthritis), psoriatic arthritis, or psoriasis and related diseases and disorders. In certain embodiments, the disease or disorder is psoriasis (eg, psoriasis vulgaris, guttate psoriasis), inverse psoriasis, pustular psoriasis, palmo-plantar, Psoriasis, psoriasis vulgaris, or psoriatic psoriasis, atopic dermatitis, acne ectopica, ulcerative colitis, Crohn's disease, Celiac's disease Sexual sprue, enteropathy with seronegative arthritis, microscopic colitis, collagen-accumulating colitis, eosinophilia gastroenteritis/esophagitis, colitis associated with radiation or chemotherapy, leukocyte adhesion deficiency-1 Psoriasis with congenital immunity disorders, chronic granulomatosis, glycogenosis type 1b, Hermannsky-Padrac syndrome, Chediac-East syndrome, Wiscot-Aldrich syndrome, rectal colon resection and ileal anastomosis, Later ileal psoriasis, gastrointestinal cancer, pancreatitis, insulin-dependent diabetes, mammary psoriasis, psoriasis, bile ductitis, primary biliary cirrhosis, virus-related enteropathy, periductitis, chronic bronchitis, chronic sinusitis, asthma, Psoriasis, or implant-to-host disease. In some embodiments, the disease or disorder is selected from subset of inflammatory diseases, which include, but are not limited to psoriasis (PSO), psoriatic arthritis (PSA), inflammatory bowel disease (IBD), Crohn's disease (CD), or ulcerative colitis (UC). In certain embodiments, the subject is a subject in need thereof. In some embodiments, the subject has the disease or condition. In other embodiments, the subject is at risk of having the disease or condition. In some embodiments, the administration results in the prevention or treatment of the disease or condition.

In another embodiment, provided herein is a method of preventing and/or treating a symptom of a disease or condition in a subject, comprising administering an effective amount of an antibody or antigen binding fragment thereof provided herein. In one embodiment, provided herein is a method of preventing a symptom of a disease or condition in a subject, comprising administering an effective amount of an antibody or antigen binding fragment thereof provided herein. In one embodiment, provided herein is a method of treating a symptom of a disease or condition in a subject, comprising administering an effective amount of an antibody or antigen binding fragment thereof provided herein. In some embodiments, the disease or condition is an IL-23R-mediated and/or IL-23 mediated disease. In some embodiments, the disease or disorder is associated with IL-23R. In some embodiments, the disease or disorder is an autoimmune disease. In certain embodiments, the disease or disorder is multiple sclerosis, asthma, rheumatoid arthritis, inflammatory bowel disease (IBD), juvenile IBD, adolescent IBD, Crohn's disease, sarcoidosis, systemic elitematodes, ankylosing spondylitis, Autoimmune inflammation such as inflammation (axial spondylotic arthritis), psoriatic arthritis, or psoriasis and related diseases and disorders. In certain embodiments, the disease or disorder is psoriasis (eg, psoriasis vulgaris, guttate psoriasis), inverse psoriasis, pustular psoriasis, palmo-plantar, Psoriasis, psoriasis vulgaris, or psoriatic psoriasis, atopic dermatitis, acne ectopica, ulcerative colitis, Crohn's disease, Celiac's disease Sexual sprue, enteropathy with seronegative arthritis, microscopic colitis, collagen-accumulating colitis, eosinophilia gastroenteritis/esophagitis, colitis associated with radiation or chemotherapy, leukocyte adhesion deficiency-1 Psoriasis with congenital immunity disorders, chronic granulomatosis, glycogenosis type 1b, Hermannsky-Padrac syndrome, Chediac-East syndrome, Wiscot-Aldrich syndrome, rectal colon resection and ileal anastomosis, Later ileal psoriasis, gastrointestinal cancer, pancreatitis, insulin-dependent diabetes, mammary psoriasis, psoriasis, bile ductitis, primary biliary cirrhosis, virus-related enteropathy, periductitis, chronic bronchitis, chronic sinusitis, asthma, Psoriasis, or implant-to-host disease. In some embodiments, the disease or disorder is selected from subset of inflammatory diseases, which include, but are not limited to psoriasis (PSO), psoriatic arthritis (PSA), inflammatory bowel disease (IBD), Crohn's disease (CD), or ulcerative colitis (UC). In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

Also provided herein are methods of preventing and/or treating a disease or condition by administrating to a subject of an effective amount of an antibody or antigen binding fragment thereof provided herein, or pharmaceutical composition comprising an antibody or antigen binding fragment thereof provided herein. In one aspect, the antibody or antigen binding fragment thereof is substantially purified (i.e., substantially free from substances that limit its effect or produce undesired side-effects). The subject administered a therapy can be a mammal such as non-primate (e.g., cows, pigs, horses, cats, dogs, rats etc.) or a primate (e.g., a monkey, such as a cynomolgus macaque monkey, or a human). In a one embodiment, the subject is a human. In another embodiment, the subject is a human with a disease or condition.

Various delivery systems are known and can be used to administer a prophylactic or therapeutic agent (e.g., an antibody or antigen binding fragment thereof provided herein), including, but not limited to, encapsulation in liposomes, microparticles, microcapsules, recombinant cells capable of expressing the antibody or antigen binding fragment thereof, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent (e.g., an antibody or antigen binding fragment thereof provided herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., an antibody or antigen binding fragment thereof provided herein), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

In some embodiment, nanoparticles that contain anti-IL-23R antibodies are used to deliver therapeutic agents to IL-23R expressing cells. In some embodiment, the therapeutic agents antagonize a different target other than IL-23R (e.g. siRNA against TNFa, etc).

In a specific embodiment, it may be desirable to administer a prophylactic or therapeutic agent, or a pharmaceutical composition provided herein locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as sialastic membranes, or fibers. In some embodiments, when administering an antibody or antigen binding fragment thereof provided herein, care must be taken to use materials to which the antibody or antigen binding fragment thereof does not absorb.

In another embodiment, a prophylactic or therapeutic agent, or a composition provided herein can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In another embodiment, a prophylactic or therapeutic agent, or a composition provided herein can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of a prophylactic or therapeutic agent (e.g., an antibody provided herein) or a composition provided herein (see e.g., Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the nasal passages or lungs, thus requiring only a fraction of the systemic dose (see, e.g., Goodson, in Medical Applications of Controlled Release, supra, vol. 2, pp. 115-138 (1984)). Controlled release systems are discussed in the review by Langer (1990, Science 249:1527-1533). Any technique known to one of skill in the art can be used to produce sustained release formulations comprising one or more antibody or antigen binding fragment thereof provided herein. See, e.g., U.S. Pat. No. 4,526,938, PCT publication WO 91/05548, PCT publication WO 96/20698, Ning et al., 1996, “Intratumoral Radioimmunotherapy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment, where the composition provided herein is a nucleic acid encoding a prophylactic or therapeutic agent (e.g., an antibody or antigen binding fragment thereof provided herein), the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (see, e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

In a specific embodiment, a composition provided herein comprises one, two or more antibodies or antigen binding fragments thereof provided herein. In another embodiment, a composition provided herein comprises one, two or more antibodies or antigen binding fragments thereof provided herein and a prophylactic or therapeutic agent other than an antibody or antigen binding fragment thereof provided herein. In one embodiment, the agents are known to be useful for or have been or are currently used for the prevention, management, treatment and/or amelioration of a disease or condition. In addition to prophylactic or therapeutic agents, the compositions provided herein may also comprise an excipient.

The compositions provided herein include bulk drug compositions useful in the manufacture of pharmaceutical compositions (e.g., compositions that are suitable for administration to a subject or patient) that can be used in the preparation of unit dosage forms. In an embodiment, a composition provided herein is a pharmaceutical composition. Such compositions comprise a prophylactically or therapeutically effective amount of one or more prophylactic or therapeutic agents (e.g., an antibody or antigen binding fragment thereof provided herein or other prophylactic or therapeutic agent), and a pharmaceutically acceptable excipient. The pharmaceutical compositions can be formulated to be suitable for the route of administration to a subject.

In a specific embodiment, the term “excipient” can also refer to a diluent, adjuvant (e.g., Freunds' adjuvant (complete or incomplete) or vehicle. Pharmaceutical excipients can be sterile liquids, such as water and oils, including those of petroleum, animal, vegetable or synthetic origin, such as peanut oil, soybean oil, mineral oil, sesame oil and the like. Water is an exemplary excipient when the pharmaceutical composition is administered intravenously. Saline solutions and aqueous dextrose and glycerol solutions can also be employed as liquid excipients, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard excipients such as pharmaceutical grades of mannitol, lactose, starch, magnesium stearate, sodium saccharine, cellulose, magnesium carbonate, etc. Examples of suitable pharmaceutical excipients are described in Remington's Pharmaceutical Sciences (1990) Mack Publishing Co., Easton, PA. Such compositions will contain a prophylactically or therapeutically effective amount of the antibody or antigen binding fragment thereof provided herein, such as in purified form, together with a suitable amount of excipient so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Such compositions, however, may be administered by a route other than intravenous.

Generally, the ingredients of compositions provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

An antibody or antigen binding fragment thereof provided herein can be packaged in a hermetically sealed container such as an ampoule or sachette indicating the quantity of antibody. In one embodiment, the antibody or antigen binding fragment thereof is supplied as a dry sterilized lyophilized powder or water free concentrate in a hermetically sealed container and can be reconstituted, e.g., with water or saline to the appropriate concentration for administration to a subject. The lyophilized antibody or antigen binding fragment thereof can be stored at between 2 and 8° C. in its original container and the antibody or antigen binding fragment thereof can be administered within 12 hours, such as within 6 hours, within 5 hours, within 3 hours, or within 1 hour after being reconstituted. In an alternative embodiment, an antibody or antigen binding fragment thereof provided herein is supplied in liquid form in a hermetically sealed container indicating the quantity and concentration of the antibody.

The compositions provided herein can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a prophylactic or therapeutic agent (e.g., an antibody or antigen binding fragment thereof provided herein), or a composition provided herein that will be effective in the prevention and/or treatment of a disease or condition can be determined by standard clinical techniques. In addition, in vitro assays may optionally be employed to help identify optimal dosage ranges. The precise dose to be employed in the formulation will also depend on the route of administration, and the seriousness of a disease or condition, and should be decided according to the judgment of the practitioner and each patient's circumstances.

Effective doses may be extrapolated from dose-response curves derived from in vitro or animal model test systems.

In certain embodiments, the route of administration for a dose of an antibody or antigen binding fragment thereof provided herein to a patient is intranasal, intramuscular, intravenous, subcutaneous, or a combination thereof, but other routes described herein are also acceptable. Each dose may or may not be administered by an identical route of administration. In some embodiments, an antibody or antigen binding fragment thereof provided herein may be administered via multiple routes of administration simultaneously or subsequently to other doses of the same or a different antibody or antigen binding fragment thereof provided herein.

In certain embodiments, the antibody or antigen binding fragment thereof provided herein are administered prophylactically or therapeutically to a subject. The antibody or antigen binding fragment thereof provided herein can be prophylactically or therapeutically administered to a subject so as to prevent, lessen or ameliorate a disease or symptom thereof.

### Pharmaceutical Compositions

In one aspect, the present disclosure further provides pharmaceutical compositions comprising at least one antibody or antigen binding fragment thereof of the present disclosure. In some embodiments, a pharmaceutical composition comprises therapeutically effective amount of an antibody or antigen binding fragment thereof provided herein and a pharmaceutically acceptable excipient.

Pharmaceutical compositions comprising an antibody or antigen binding fragment thereof are prepared for storage by mixing the fusion protein having the desired degree of purity with optional physiologically acceptable excipients (see, e.g., Remington, Remington's Pharmaceutical Sciences (18th ed. 1980)) in the form of aqueous solutions or lyophilized or other dried forms.

The antibody or antigen binding fragment thereof of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

An antibody or antigen binding fragment thereof provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antibody or antigen binding fragment thereof as described herein, including, but not limited to, encapsulation in liposomes, microparticles, microcapsules, recombinant cells capable of expressing the antibody or antigen binding fragment thereof, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody or antigen binding fragment thereof as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibody or antigen binding fragment thereof as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### Gene Therapy

In a specific embodiment, nucleic acids comprising sequences encoding antibodies or functional derivatives thereof, are administered to a subject for use in a method provided herein, for example, to prevent, manage, treat and/or ameliorate an IL-23R-mediated disease, disorder or condition, by way of gene therapy. Such therapy encompasses that performed by the administration to a subject of an expressed or expressible nucleic acid. In an embodiment, the nucleic acids produce their encoded antibody, and the antibody mediates a prophylactic or therapeutic effect.

Any of the methods for recombinant gene expression (or gene therapy) available in the art can be used.

For general review of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a specific embodiment, a composition comprises nucleic acids encoding an antibody provided herein, the nucleic acids being part of an expression vector that expresses the antibody or chimeric proteins or heavy or light chains thereof in a suitable host. In particular, such nucleic acids have promoters, such as heterologous promoters, operably linked to the antibody coding region, the promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438).

Delivery of the nucleic acids into a subject can be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where the sequences are expressed to produce the encoded product. This can be accomplished by any of numerous methods known in the art, e.g., by constructing them as part of an appropriate nucleic acid expression vector and administering the vector so that the sequences become intracellular, e.g., by infection using defective or attenuated retroviral or other viral vectors (see U.S. Pat. No. 4,980,286), or by direct injection of naked DNA, or by use of microparticle bombardment (e.g., a gene gun; Biolistic, Dupont), or coating with lipids or cell surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO 92/20316; WO93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding an antibody are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy can be cloned into one or more vectors, which facilitates delivery of the gene into a subject. More detail about retroviral vectors can be found in Boesen et al., 1994, Biotherapy 6:291-302, which describes the use of a retroviral vector to deliver the MDR1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in the recombinant production of antibodies. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. In a specific embodiment, adenovirus vectors are used.

Adeno-associated virus (AAV) can also be utilized (Walsh et al., 1993, Proc. Soc. Exp. Biol. Med. 204:289-300; and U.S. Pat. No. 5,436,146). In a specific embodiment, AAV vectors are used to express an anti-IL-23R antibody as provided herein. In certain embodiments, the AAV comprises a nucleic acid encoding a VH domain. In other embodiments, the AAV comprises a nucleic acid encoding a VL domain. In certain embodiments, the AAV comprises a nucleic acid encoding a VH domain and a VL domain. In some embodiments of the methods provided herein, a subject is administered an AAV comprising a nucleic acid encoding a VH domain and an AAV comprising a nucleic acid encoding a VL domain. In other embodiments, a subject is administered an AAV comprising a nucleic acid encoding a VH domain and a VL domain. In certain embodiments, the VH and VL domains are over-expressed.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and can be used in accordance with the methods provided herein, provided that the necessary developmental and physiological functions of the recipient cells are not disrupted. The technique should provide for the stable transfer of the nucleic acid to the cell, so that the nucleic acid is expressible by the cell, such as heritable and expressible by its cell progeny.

The resulting recombinant cells can be delivered to a subject by various methods known in the art. Recombinant blood cells (e.g., hematopoietic stem or progenitor cells) can be administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a specific embodiment, the cell used for gene therapy is autologous to the subject.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the methods provided herein (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### Kits

Also provided herein are kits comprising an antibody (e.g., an anti-IL-23R antibody) provided herein, or a composition (e.g., a pharmaceutical composition) thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Also provided herein are panels of antibodies that immunospecifically bind to an IL-23R antigen. In specific embodiments, provided herein are panels of antibodies having different association rate constants, different dissociation rate constants, different affinities for IL-23R antigen, and/or different specificities for an IL-23R antigen. In certain embodiments, provided herein are panels of about 10, preferably about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96 well or 384 well plates, such as for assays such as ELISAs.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges include combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

### ASPECTS OF THE PRESENT INVENTION

The following aspects illustrate and are not intended to limit scope of the present invention. Instead, these aspects provide guidance to any skilled artisan on how to prepare and use compounds, compositions and methods taught by the present invention, where such skilled artisans will appreciate that modifications may be made without departing from the spirit and scope of the invention.

1. An isolated monoclonal antibody or antigen binding fragment thereof that binds human interleukin-23 receptor (IL-23R) on a mammalian cell, wherein the antibody or fragment comprises:
a. an HCDR1, an HCDR2, and an HCDR3 as set forth in SEQ ID NO:236, and an LCDR1, an LCDR2, and an LCDR3 as set forth in SEQ ID NO:237;
b. an HCDR1, an HCDR2, and an HCDR3 as set forth in SEQ ID NO:798, and an LCDR1, an LCDR2, and an LCDR3 as set forth in SEQ ID NO:799;
c. an HCDR1, an HCDR2, and an HCDR3 as set forth in SEQ ID NO:792, and an LCDR1, an LCDR2, and an LCDR3 as set forth in SEQ ID NO:793;
d. an HCDR1, an HCDR2, and an HCDR3 as set forth in SEQ ID NO:786, and an LCDR1, an LCDR2, and an LCDR3 as set forth in SEQ ID NO:787;
e. an HCDR1, an HCDR2, and an HCDR3 as set forth in SEQ ID NO:138, and an LCDR1, an LCDR2, and an LCDR3 as set forth in SEQ ID NO:139;
f. an HCDR1, an HCDR2, and an HCDR3 as set forth in SEQ ID NO:144, and an LCDR1, an LCDR2, and an LCDR3 as set forth in SEQ ID NO:145;
g. an HCDR1, an HCDR2, and an HCDR3 as set forth in SEQ ID NO:152, and an LCDR1, an LCDR2, and an LCDR3 as set forth in SEQ ID NO:153;
h. an HCDR1, an HCDR2, and an HCDR3 as set forth in SEQ ID NO:154, and an LCDR1, an LCDR2, and an LCDR3 as set forth in SEQ ID NO:155;
i. an HCDR1, an HCDR2, and an HCDR3 as set forth in SEQ ID NO:156, and an LCDR1, an LCDR2, and an LCDR3 as set forth in SEQ ID NO:157;
j. an HCDR1, an HCDR2, and an HCDR3 as set forth in SEQ ID NO:160, and an LCDR1, an LCDR2, and an LCDR3 as set forth in SEQ ID NO:161;
k. an HCDR1, an HCDR2, and an HCDR3 as set forth in SEQ ID NO:164, and an LCDR1, an LCDR2, and an LCDR3 as set forth in SEQ ID NO:165;
l. an HCDR1, an HCDR2, and an HCDR3 as set forth in SEQ ID NO:166, and an LCDR1, an LCDR2, and an LCDR3 as set forth in SEQ ID NO:167;
m. an HCDR1, an HCDR2, and an HCDR3 as set forth in SEQ ID NO:168, and an LCDR1, an LCDR2, and an LCDR3 as set forth in SEQ ID NO:169;
n. an HCDR1, an HCDR2, and an HCDR3 as set forth in SEQ ID NO:174, and an LCDR1, an LCDR2, and an LCDR3 as set forth in SEQ ID NO:175;
o. an HCDR1, an HCDR2, and an HCDR3 as set forth in SEQ ID NO:788, and an LCDR1, an LCDR2, and an LCDR3 as set forth in SEQ ID NO:789;
p. an HCDR1, an HCDR2, and an HCDR3 as set forth in SEQ ID NO:186, and an LCDR1, an LCDR2, and an LCDR3 as set forth in SEQ ID NO:187;
q. an HCDR1, an HCDR2, and an HCDR3 as set forth in SEQ ID NO:828, and an LCDR1, an LCDR2, and an LCDR3 as set forth in SEQ ID NO:829;
r. an HCDR1, an HCDR2, and an HCDR3 as set forth in SEQ ID NO:790, and an LCDR1, an LCDR2, and an LCDR3 as set forth in SEQ ID NO:791;
s. an HCDR1, an HCDR2, and an HCDR3 as set forth in SEQ ID NO:188, and an LCDR1, an LCDR2, and an LCDR3 as set forth in SEQ ID NO:189;
t. an HCDR1, an HCDR2, and an HCDR3 as set forth in SEQ ID NO:884, and an LCDR1, an LCDR2, and an LCDR3 as set forth in SEQ ID NO:885;
u. an HCDR1, an HCDR2, and an HCDR3 as set forth in SEQ ID NO:192, and an LCDR1, an LCDR2, and an LCDR3 as set forth in SEQ ID NO:193;
v. an HCDR1, an HCDR2, and an HCDR3 as set forth in SEQ ID NO:194, and an LCDR1, an LCDR2, and an LCDR3 as set forth in SEQ ID NO:195;
w. an HCDR1, an HCDR2, and an HCDR3 as set forth in SEQ ID NO:198, and an LCDR1, an LCDR2, and an LCDR3 as set forth in SEQ ID NO:199;
x. an HCDR1, an HCDR2, and an HCDR3 as set forth in SEQ ID NO:200, and an LCDR1, an LCDR2, and an LCDR3 as set forth in SEQ ID NO:201;
y. an HCDR1, an HCDR2, and an HCDR3 as set forth in SEQ ID NO:202, and an LCDR1, an LCDR2, and an LCDR3 as set forth in SEQ ID NO:203;
z. an HCDR1, an HCDR2, and an HCDR3 as set forth in SEQ ID NO:204, and an LCDR1, an LCDR2, and an LCDR3 as set forth in SEQ ID NO:205;
aa. an HCDR1, an HCDR2, and an HCDR3 as set forth in SEQ ID NO:206, and an LCDR1, an LCDR2, and an LCDR3 as set forth in SEQ ID NO:207;
bb. an HCDR1, an HCDR2, and an HCDR3 as set forth in SEQ ID NO:216, and an LCDR1, an LCDR2, and an LCDR3 as set forth in SEQ ID NO:217;
cc. an HCDR1, an HCDR2, and an HCDR3 as set forth in SEQ ID NO:222, and an LCDR1, an LCDR2, and an LCDR3 as set forth in SEQ ID NO:223;
dd. an HCDR1, an HCDR2, and an HCDR3 as set forth in SEQ ID NO:230, and an LCDR1, an LCDR2, and an LCDR3 as set forth in SEQ ID NO:231;
ee. an HCDR1, an HCDR2, and an HCDR3 as set forth in SEQ ID NO:232, and an LCDR1, an LCDR2, and an LCDR3 as set forth in SEQ ID NO:233;
ff. an HCDR1, an HCDR2, and an HCDR3 as set forth in SEQ ID NO:234, and an LCDR1, an LCDR2, and an LCDR3 as set forth in SEQ ID NO:235;
gg. an HCDR1, an HCDR2, and an HCDR3 as set forth in SEQ ID NO:238, and an LCDR1, an LCDR2, and an LCDR3 as set forth in SEQ ID NO:239;
hh. an HCDR1, an HCDR2, and an HCDR3 as set forth in SEQ ID NO:246, and an LCDR1, an LCDR2, and an LCDR3 as set forth in SEQ ID NO:247;
ii. an HCDR1, an HCDR2, and an HCDR3 as set forth in SEQ ID NO:248, and an LCDR1, an LCDR2, and an LCDR3 as set forth in SEQ ID NO:249;
jj. an HCDR1, an HCDR2, and an HCDR3 as set forth in SEQ ID NO:254, and an LCDR1, an LCDR2, and an LCDR3 as set forth in SEQ ID NO:255;
kk. an HCDR1, an HCDR2, and an HCDR3 as set forth in SEQ ID NO:260, and an LCDR1, an LCDR2, and an LCDR3 as set forth in SEQ ID NO:261;
ll. an HCDR1, an HCDR2, and an HCDR3 as set forth in SEQ ID NO:262, and a LCDR1, an LCDR2, and an LCDR3 as set forth in SEQ ID NO:263; or
mm. an HCDR1, an HCDR2, and an HCDR3 as set forth in SEQ ID NO:264, and an LCDR1, an LCDR2, and an LCDR3 as set forth in SEQ ID NO:265.

2. The isolated monoclonal antibody or antigen binding fragment thereof of aspect 1 that binds human interleukin-23 receptor (IL-23R) on a mammalian cell, wherein the antibody or fragment comprises:
nn. a VH comprising an HCDR1, an HCDR2, and an HCDR3 having an amino acid sequence of SEQ ID NOs:570, 571, and 572, respectively, and a VL comprising an LCDR1, an LCDR2, and an LCDR3 having an amino acid sequence of SEQ ID NOs:573, 574, and 575, respectively;
oo. a VH comprising an HCDR1, an HCDR2, and an HCDR3 having an amino acid sequence of SEQ ID NOs:936, 937, and 938, respectively, and a VL comprising an LCDR1, an LCDR2, and an LCDR3 having an amino acid sequence of SEQ ID NOs:939, 940, and 941, respectively;
pp. a VH comprising an HCDR1, an HCDR2, and an HCDR3 having an amino acid sequence of SEQ ID NOs:918, 919, and 920, respectively, and a VL comprising an LCDR1, an LCDR2, and an LCDR3 having an amino acid sequence of SEQ ID NOs:921, 922, and 923, respectively;
qq. a VH comprising an HCDR1, an HCDR2, and an HCDR3 having an amino acid sequence of SEQ ID NOs:900, 901, and 902, respectively, and a VL comprising an LCDR1, an LCDR2, and an LCDR3 having an amino acid sequence of SEQ ID NOs:903, 904, and 905, respectively;
rr. a VH comprising an HCDR1, an HCDR2, and an HCDR3 having an amino acid sequence of SEQ ID NOs:276, 277, and 278, respectively, and a VL comprising an LCDR1, an LCDR2, and an LCDR3 having an amino acid sequence of SEQ ID NOs:279, 280, and 281, respectively;
ss. a VH comprising an HCDR1, an HCDR2, and an HCDR3 having an amino acid sequence of SEQ ID NOs:294, 295, and 296, respectively, and a VL comprising an LCDR1, an LCDR2, and an LCDR3 having an amino acid sequence of SEQ ID NOs:297, 298, and 299, respectively;
tt. a VH comprising an HCDR1, an HCDR2, and an HCDR3 having an amino acid sequence of SEQ ID NOs:318, 319, and 320, respectively, and a VL comprising an LCDR1, an LCDR2, and an LCDR3 having an amino acid sequence of SEQ ID NOs:321, 322, and 323, respectively;
uu. a VH comprising an HCDR1, an HCDR2, and an HCDR3 having an amino acid sequence of SEQ ID NOs:324, 325, and 326, respectively, and a VL comprising an LCDR1, an LCDR2, and an LCDR3 having an amino acid sequence of SEQ ID NOs:327, 328, and 329,
vv. a VH comprising an HCDR1, an HCDR2, and an HCDR3 having an amino acid sequence of SEQ ID NOs:330, 331, and 332, respectively, and a VL comprising an LCDR1, an LCDR2, and an LCDR3 having an amino acid sequence of SEQ ID NOs:333, 334, and 335, respectively;
ww. a VH comprising an HCDR1, an HCDR2, and an HCDR3 having an amino acid sequence of SEQ ID NOs:342, 343, and 344, respectively, and a VL comprising an LCDR1, an LCDR2, and an LCDR3 having an amino acid sequence of SEQ ID NOs:345, 346, and 347, respectively;
xx. a VH comprising an HCDR1, an HCDR2, and an HCDR3 having an amino acid sequence of SEQ ID NOs:354, 355, and 356, respectively, and a VL comprising an LCDR1, an LCDR2, and an LCDR3 having an amino acid sequence of SEQ ID NOs:357, 358, and 359, respectively;
yy. a VH comprising an HCDR1, an HCDR2, and an HCDR3 having an amino acid sequence of SEQ ID NOs:360, 361, and 362, respectively, and a VL comprising an LCDR1, an LCDR2, and an LCDR3 having an amino acid sequence of SEQ ID NOs:363, 364, and 365, respectively;
zz. a VH comprising an HCDR1, an HCDR2, and an HCDR3 having an amino acid sequence of SEQ ID NOs:366, 367, and 368, respectively, and a VL comprising an LCDR1, an LCDR2, and an LCDR3 having an amino acid sequence of SEQ ID NOs:369, 370, and 371, respectively;
aaa. a VH comprising an HCDR1, an HCDR2, and an HCDR3 having an amino acid sequence of SEQ ID NOs:384, 385, and 386, respectively, and a VL comprising an LCDR1, an LCDR2, and an LCDR3 having an amino acid sequence of SEQ ID NOs:387, 388, and 389, respectively;
bbb. a VH comprising an HCDR1, an HCDR2, and an HCDR3 having an amino acid sequence of SEQ ID NOs:906, 907, and 908, respectively, and a VL comprising an LCDR1, an LCDR2, and an LCDR3 having an amino acid sequence of SEQ ID NOs:909, 910, and 911, respectively;
ccc. a VH comprising an HCDR1, an HCDR2, and an HCDR3 having an amino acid sequence of SEQ ID NOs:420, 421, and 422, respectively, and a VL comprising an LCDR1, an LCDR2, and an LCDR3 having an amino acid sequence of SEQ ID NOs:423, 424, and 425, respectively;
ddd. a VH comprising an HCDR1, an HCDR2, and an HCDR3 having an amino acid sequence of SEQ ID NOs:1026, 1027, and 1028, respectively, and a VL comprising an LCDR1, an LCDR2, and an LCDR3 having an amino acid sequence of SEQ ID NOs:1029, 1030, and 1031,
eee. a VH comprising an HCDR1, an HCDR2, and an HCDR3 having an amino acid sequence of SEQ ID NOs:912, 913, and 914, respectively, and a VL comprising an LCDR1, an LCDR2, and an LCDR3 having an amino acid sequence of SEQ ID NOs:915, 916, and 917, respectively;
fff. a VH comprising an HCDR1, an HCDR2, and an HCDR3 having an amino acid sequence of SEQ ID NOs:426, 427, and 428, respectively, and a VL comprising an LCDR1, an LCDR2, and an LCDR3 having an amino acid sequence of SEQ ID NOs:429, 430, and 431, respectively;
ggg. a VH comprising an HCDR1, an HCDR2, and an HCDR3 having an amino acid sequence of SEQ ID NOs:1194, 1195, and 1196, respectively, and a VL comprising an LCDR1, an LCDR2, and an LCDR3 having an amino acid sequence of SEQ ID NOs:1197, 1198, and 1199, respectively;
hhh. a VH comprising an HCDR1, an HCDR2, and an HCDR3 having an amino acid sequence of SEQ ID NOs:438, 439, and 440, respectively, and a VL comprising an LCDR1, an LCDR2, and an LCDR3 having an amino acid sequence of SEQ ID NOs:441, 442, and 443, respectively;
iii. a VH comprising an HCDR1, an HCDR2, and an HCDR3 having an amino acid sequence of SEQ ID NOs:444, 445, and 446, respectively, and a VL comprising an LCDR1, an LCDR2, and an LCDR3 having an amino acid sequence of SEQ ID NOs:447, 448, and 449, respectively;
jjj. a VH comprising an HCDR1, an HCDR2, and an HCDR3 having an amino acid sequence of SEQ ID NOs:456, 457, and 458, respectively, and a VL comprising an LCDR1, an LCDR2, and an LCDR3 having an amino acid sequence of SEQ ID NOs:459, 460, and 461, respectively;
kkk. a VH comprising an HCDR1, an HCDR2, and an HCDR3 having an amino acid sequence of SEQ ID NOs:462, 463, and 464, respectively, and a VL comprising an LCDR1, an LCDR2, and an LCDR3 having an amino acid sequence of SEQ ID NOs:465, 466, and 467, respectively;
lll. a VH comprising an HCDR1, an HCDR2, and an HCDR3 having an amino acid sequence of SEQ ID NOs:468, 469, and 470, respectively, and a VL comprising an LCDR1, an LCDR2, and an LCDR3 having an amino acid sequence of SEQ ID NOs:471, 472, and 473, respectively;
mmm. a VH comprising an HCDR1, an HCDR2, and an HCDR3 having an amino acid sequence of SEQ ID NOs:474, 475, and 476, respectively, and a VL comprising an LCDR1, an LCDR2, and an LCDR3 having an amino acid sequence of SEQ ID NOs:477, 478, and 479,
nnn. a VH comprising an HCDR1, an HCDR2, and an HCDR3 having an amino acid sequence of SEQ ID NOs:480, 481, and 482, respectively, and a VL comprising an LCDR1, an LCDR2, and an LCDR3 having an amino acid sequence of SEQ ID NOs:483, 484, and 485, respectively;
ooo. a VH comprising an HCDR1, an HCDR2, and an HCDR3 having an amino acid sequence of SEQ ID NOs:510, 511, and 512, respectively, and a VL comprising an LCDR1, an LCDR2, and an LCDR3 having an amino acid sequence of SEQ ID NOs:513, 514, and 515, respectively;
ppp. a VH comprising an HCDR1, an HCDR2, and an HCDR3 having an amino acid sequence of SEQ ID NOs:528, 529, and 530, respectively, and a VL comprising an LCDR1, an LCDR2, and an LCDR3 having an amino acid sequence of SEQ ID NOs:531, 532, and 533, respectively;
qqq. a VH comprising an HCDR1, an HCDR2, and an HCDR3 having an amino acid sequence of SEQ ID NOs:552, 553, and 554, respectively, and a VL comprising an LCDR1, an LCDR2, and an LCDR3 having an amino acid sequence of SEQ ID NOs:555, 556, and 557, respectively;
rrr. a VH comprising an HCDR1, an HCDR2, and an HCDR3 having an amino acid sequence of SEQ ID NOs:558, 559, and 560, respectively, and a VL comprising an LCDR1, an LCDR2, and an LCDR3 having an amino acid sequence of SEQ ID NOs:561, 562, and 563, respectively;
sss. a VH comprising an HCDR1, an HCDR2, and an HCDR3 having an amino acid sequence of SEQ ID NOs:564, 565, and 566, respectively, and a VL comprising an LCDR1, an LCDR2, and an LCDR3 having an amino acid sequence of SEQ ID NOs:567, 568, and 569, respectively;
ttt. a VH comprising an HCDR1, an HCDR2, and an HCDR3 having an amino acid sequence of SEQ ID NOs:576, 577, and 578, respectively, and a VL comprising an LCDR1, an LCDR2, and an LCDR3 having an amino acid sequence of SEQ ID NOs:579, 580, and 581, respectively;
uuu. a VH comprising an HCDR1, an HCDR2, and an HCDR3 having an amino acid sequence of SEQ ID NOs:600, 601, and 602, respectively, and a VL comprising an LCDR1, an LCDR2, and an LCDR3 having an amino acid sequence of SEQ ID NOs:603, 604, and 605, respectively;
vvv. a VH comprising an HCDR1, an HCDR2, and an HCDR3 having an amino acid sequence of SEQ ID NOs:606, 607, and 608, respectively, and a VL comprising an LCDR1, an LCDR2, and an LCDR3 having an amino acid sequence of SEQ ID NOs:609, 610, and 611,
www. a VH comprising an HCDR1, an HCDR2, and an HCDR3 having an amino acid sequence of SEQ ID NOs:624, 625, and 626, respectively, and a VL comprising an LCDR1, an LCDR2, and an LCDR3 having an amino acid sequence of SEQ ID NOs:627, 628, and 629, respectively;
xxx. a VH comprising an HCDR1, an HCDR2, and an HCDR3 having an amino acid sequence of SEQ ID NOs:642, 643, and 644, respectively, and a VL comprising an LCDR1, an LCDR2, and an LCDR3 having an amino acid sequence of SEQ ID NOs:645, 646, and 647, respectively;
yyy. a VH comprising an HCDR1, an HCDR2, and an HCDR3 having an amino acid sequence of SEQ ID NOs:648, 649, and 650, respectively, and a VL comprising an LCDR1, an LCDR2, and an LCDR3 having an amino acid sequence of SEQ ID NOs:651, 652, and 653, respectively; or
zzz. a VH comprising an HCDR1, an HCDR2, and an HCDR3 having an amino acid sequence of SEQ ID NOs:654, 655, and 656, respectively, and a VL comprising an LCDR1, an LCDR2, and an LCDR3 having an amino acid sequence of SEQ ID NOs:657, 658, and 659, respectively.

3. The isolated monoclonal antibody or antigen binding fragment thereof of aspect 1 or 2 that binds human interleukin-23 receptor (IL-23R) on a mammalian cell, wherein the antibody or fragment comprises:
a. a VH comprising the amino acid sequence of SEQ ID NO:236, and a VL comprising the amino acid sequence of SEQ ID NO:237;
b. a VH comprising the amino acid sequence of SEQ ID NO:798, and a VL comprising the amino acid sequence of SEQ ID NO:799;
c. a VH comprising the amino acid sequence of SEQ ID NO:792, and a VL comprising the amino acid sequence of SEQ ID NO:793;
d. a VH comprising the amino acid sequence of SEQ ID NO:786, and a VL comprising the amino acid sequence of SEQ ID NO:787;
e. a VH comprising the amino acid sequence of SEQ ID NO:138, and a VL comprising the amino acid sequence of SEQ ID NO:139;
f. a VH comprising the amino acid sequence of SEQ ID NO:144, and a VL comprising the amino acid sequence of SEQ ID NO:145;
g. a VH comprising the amino acid sequence of SEQ ID NO:152, and a VL comprising the amino acid sequence of SEQ ID NO:153;
h. a VH comprising the amino acid sequence of SEQ ID NO:154, and a VL comprising the amino acid sequence of SEQ ID NO:155;
i. a VH comprising the amino acid sequence of SEQ ID NO:156, and a VL comprising the amino acid sequence of SEQ ID NO:157;
j. a VH comprising the amino acid sequence of SEQ ID NO:160, and a VL comprising the amino acid sequence of SEQ ID NO:161;
k. a VH comprising the amino acid sequence of SEQ ID NO:164, and a VL comprising the amino acid sequence of SEQ ID NO:165;
l. a VH comprising the amino acid sequence of SEQ ID NO:166, and a VL comprising the amino acid sequence of SEQ ID NO:167;
m. a VH comprising the amino acid sequence of SEQ ID NO:168, and a VL comprising the amino acid sequence of SEQ ID NO:169;
n. a VH comprising the amino acid sequence of SEQ ID NO:174, and a VL comprising the amino acid sequence of SEQ ID NO:175;
o. a VH comprising the amino acid sequence of SEQ ID NO:788, and a VL comprising the amino acid sequence of SEQ ID NO:789;
p. a VH comprising the amino acid sequence of SEQ ID NO:186, and a VL comprising the amino acid sequence of SEQ ID NO:187;
q. a VH comprising the amino acid sequence of SEQ ID NO:828, and a VL comprising the amino acid sequence of SEQ ID NO:829;
r. a VH comprising the amino acid sequence of SEQ ID NO:790, and a VL comprising the amino acid sequence of SEQ ID NO:791;
s. a VH comprising the amino acid sequence of SEQ ID NO:188, and a VL comprising the amino acid sequence of SEQ ID NO:189;
t. a VH comprising the amino acid sequence of SEQ ID NO:884, and a VL comprising the amino acid sequence of SEQ ID NO:885;
u. a VH comprising the amino acid sequence of SEQ ID NO:192, and a VL comprising the amino acid sequence of SEQ ID NO:193;
v. a VH comprising the amino acid sequence of SEQ ID NO:194, and a VL comprising the amino acid sequence of SEQ ID NO:195;
w. a VH comprising the amino acid sequence of SEQ ID NO:198, and a VL comprising the amino acid sequence of SEQ ID NO:199;
x. a VH comprising the amino acid sequence of SEQ ID NO:200, and a VL comprising the amino acid sequence of SEQ ID NO:201;
y. a VH comprising the amino acid sequence of SEQ ID NO:202, and a VL comprising the amino acid sequence of SEQ ID NO:203;
z. a VH comprising the amino acid sequence of SEQ ID NO:204, and a VL comprising the amino acid sequence of SEQ ID NO:205;
aa. a VH comprising the amino acid sequence of SEQ ID NO:206, and a VL comprising the amino acid sequence of SEQ ID NO:207;
bb. a VH comprising the amino acid sequence of SEQ ID NO:216, and a VL comprising the amino acid sequence of SEQ ID NO:217;
cc. a VH comprising the amino acid sequence of SEQ ID NO:222, and a VL comprising the amino acid sequence of SEQ ID NO:223;
dd. a VH comprising the amino acid sequence of SEQ ID NO:230, and a VL comprising the amino acid sequence of SEQ ID NO:231;
ee. a VH comprising the amino acid sequence of SEQ ID NO:232, and a VL comprising the amino acid sequence of SEQ ID NO:233;
ff. a VH comprising the amino acid sequence of SEQ ID NO:234, and a VL comprising the amino acid sequence of SEQ ID NO:235;
gg. a VH comprising the amino acid sequence of SEQ ID NO:238, and a VL comprising the amino acid sequence of SEQ ID NO:239;
hh. a VH comprising the amino acid sequence of SEQ ID NO:246, and a VL comprising the amino acid sequence of SEQ ID NO:247;
ii. a VH comprising the amino acid sequence of SEQ ID NO:248, and a VL comprising the amino acid sequence of SEQ ID NO:249;
jj. a VH comprising the amino acid sequence of SEQ ID NO:254, and a VL comprising the amino acid sequence of SEQ ID NO:255;
kk. a VH comprising the amino acid sequence of SEQ ID NO:260, and a VL comprising the amino acid sequence of SEQ ID NO:261;
ll. a VH comprising the amino acid sequence of SEQ ID NO:262, and a VL comprising the amino acid sequence of SEQ ID NO:263; or
mm. a VH comprising the amino acid sequence of SEQ ID NO:264, and a VL comprising the amino acid sequence of SEQ ID NO:265.

4. An isolated monoclonal antibody or active fragment thereof that binds human interleukin-23 receptor (IL-23R) on a mammalian cell of aspect 1, wherein the antibody or fragment comprises:
a. an HCDR1, and HCDR2 and an HCDR3 as set forth in SEQ ID NO:786, and an LCDR1, LCDR2 and LCDR3 set forth in SEQ ID NO:787;
b. an HCDR1, and HCDR2 and an HCDR3 as set forth in SEQ ID NO:790, and an LCDR1, LCDR2 and LCDR3 set forth in SEQ ID NO:791;
c. an HCDR1, and HCDR2 and an HCDR3 as set forth in SEQ ID NO:792, and an LCDR1, LCDR2 and LCDR3 set forth in SEQ ID NO:793;
d. an HCDR1, and HCDR2 and an HCDR3 as set forth in SEQ ID NO:798, and an LCDR1, LCDR2 and LCDR3 set forth in SEQ ID NO:799; or
e. an HCDR1, and HCDR2 and an HCDR3 as set forth in SEQ ID NO:236, and an LCDR1, LCDR2 and LCDR3 set forth in SEQ ID NO:237.

5. The isolated monoclonal antibody or antigen binding fragment thereof of aspect 2 that binds human interleukin-23 receptor (IL-23R) on a mammalian cell, wherein the antibody or fragment comprises:
a. a VH comprising an HCDR1, and HCDR2 and an HCDR3 having an amino acid sequence of SEQ ID NOs:570, 571, and 572, respectively and a VL comprising an LCDR1, an LCDR2, and an LCDR3 having an amino acid sequence of SEQ ID NOs:573, 574, and 575, respectively;
b. a VH comprising an HCDR1, and HCDR2 and an HCDR3 having an amino acid sequence of SEQ ID NOs:936, 937, and 938, respectively and a VL comprising an LCDR1, an LCDR2, and an LCDR3 having an amino acid sequence of SEQ ID NOs:939, 940, and 941, respectively;
c. a VH comprising an HCDR1, and HCDR2 and an HCDR3 having an amino acid sequence of SEQ ID NOs:918, 919, and 920, respectively and a VL comprising an LCDR1, an LCDR2, and an LCDR3 having an amino acid sequence of SEQ ID NOs:921, 922, and 923, respectively;
d. a VH comprising an HCDR1, and HCDR2 and an HCDR3 having an amino acid sequence of SEQ ID NOs:900, 901, and 902, respectively and a VL comprising an LCDR1, an LCDR2, and an LCDR3 having an amino acid sequence of SEQ ID NOs:903, 904, and 905, respectively; or
e. a VH comprising an HCDR1, and HCDR2 and an HCDR3 having an amino acid sequence of SEQ ID NOs:912, 913, and 914, respectively and a VL comprising an LCDR1, an LCDR2, and an LCDR3 having an amino acid sequence of SEQ ID NOs:915, 916, and 917, respectively.

6. The isolated monoclonal antibody or antigen binding fragment thereof of aspect 3 that binds human interleukin-23 receptor (IL-23R) on a mammalian cell, wherein the antibody or fragment comprises:
a. a VH comprising the amino acid sequence of SEQ ID NO:236, and a VL comprising the amino acid sequence of SEQ ID NO:237;
b. a VH comprising the amino acid sequence of SEQ ID NO:798, and a VL comprising the amino acid sequence of SEQ ID NO:799;
c. a VH comprising the amino acid sequence of SEQ ID NO:792, and a VL comprising the amino acid sequence of SEQ ID NO:793;
d. a VH comprising the amino acid sequence of SEQ ID NO:786, and a VL comprising the amino acid sequence of SEQ ID NO:787; or
e. a VH comprising the amino acid sequence of SEQ ID NO:790, and a VL comprising the amino acid sequence of SEQ ID NO:791.

7. The isolated monoclonal antibody or antigen binding fragment thereof of any of aspects 1 to 6 that binds human interleukin-23 receptor (IL-23R) on a mammalian cell, wherein the antibody or fragment comprises:
a. a VH comprising an HCDR1, and HCDR2 and an HCDR3 having an amino acid sequence of SEQ ID NOs:918, 919, and 920, respectively and a VL comprising an LCDR1, an LCDR2, and an LCDR3 having an amino acid sequence of SEQ ID NOs:921, 922, and 923, respectively;
b. a VH comprising an HCDR1, and HCDR2 and an HCDR3 having an amino acid sequence of SEQ ID NOs:936, 937, and 938, respectively and a VL comprising an LCDR1, an LCDR2, and an LCDR3 having an amino acid sequence of SEQ ID NOs:939, 940, and 941, respectively; or
c. a VH comprising an HCDR1, and HCDR2 and an HCDR3 having an amino acid sequence of SEQ ID NOs:570, 571, and 572, respectively and a VL comprising an LCDR1, an LCDR2, and an LCDR3 having an amino acid sequence of SEQ ID NOs:573, 574, and 575, respectively.

8. The isolated monoclonal antibody or antigen binding fragment thereof of any of aspects 1 to 7 that binds human interleukin-23 receptor (IL-23R) on a mammalian cell, wherein the antibody or fragment comprises:
a. a VH comprising the amino acid sequence of SEQ ID NO:792, and a VL comprising the amino acid sequence of SEQ ID NO:793;
b. a VH comprising the amino acid sequence of SEQ ID NO:798, and a VL comprising the amino acid sequence of SEQ ID NO:799; or
c. a VH comprising the amino acid sequence of SEQ ID NO:236, and a VL comprising the amino acid sequence of SEQ ID NO:237.

9. The isolated monoclonal antibody or antigen binding fragment thereof of any of aspects 1-8, wherein the mammalian cell is a human or rat cell.

10. The isolated monoclonal antibody or antigen binding fragment thereof of any of aspects 1-9, wherein the cell is a primary immune cell.

11. An isolated monoclonal antibody or antigen binding fragment thereof which recognizes human and rat interleukin-23 receptor (IL-23R), wherein the antibody or fragment comprises:
a. an HCDR1, and HCDR2 and an HCDR3 as set forth in SEQ ID NO:202, and an LCDR1, LCDR2 and LCDR3 set forth in SEQ ID NO:203;
b. an HCDR1, and HCDR2 and an HCDR3 as set forth in SEQ ID NO:252, and an LCDR1, LCDR2 and LCDR3 set forth in SEQ ID NO:253;
c. an HCDR1, and HCDR2 and an HCDR3 as set forth in SEQ ID NO:156, and an LCDR1, LCDR2 and LCDR3 set forth in SEQ ID NO:157;
d. an HCDR1, and HCDR2 and an HCDR3 as set forth in SEQ ID NO:798, and an LCDR1, LCDR2 and LCDR3 set forth in SEQ ID NO:799;
e. an HCDR1, and HCDR2 and an HCDR3 as set forth in SEQ ID NO:150, and an LCDR1, LCDR2 and LCDR3 set forth in SEQ ID NO:151;
f. an HCDR1, and HCDR2 and an HCDR3 as set forth in SEQ ID NO:204, and an LCDR1, LCDR2 and LCDR3 set forth in SEQ ID NO:205;
g. an HCDR1, and HCDR2 and an HCDR3 as set forth in SEQ ID NO:884, and an LCDR1, LCDR2 and LCDR3 set forth in SEQ ID NO:885;
h. an HCDR1, and HCDR2 and an HCDR3 as set forth in SEQ ID NO:188, and an LCDR1, LCDR2 and LCDR3 set forth in SEQ ID NO:189; or
i. an HCDR1, and HCDR2 and an HCDR3 as set forth in SEQ ID NO:212, and an LCDR1, LCDR2 and LCDR3 set forth in SEQ ID NO:213.

12. The isolated monoclonal antibody or antigen binding fragment thereof of aspect 11 which recognizes human and rat IL-23R, wherein the antibody or fragment comprises:
a. a VH comprising an HCDR1, and HCDR2 and an HCDR3 having an amino acid sequence of SEQ ID NOs:468, 469, and 470, respectively and a VL comprising an LCDR1, an LCDR2, and an LCDR3 having an amino acid sequence of SEQ ID NOs:471, 472, and 473, respectively;
b. a VH comprising an HCDR1, and HCDR2 and an HCDR3 having an amino acid sequence of SEQ ID NOs:618, 619, and 620, respectively and a VL comprising an LCDR1, an LCDR2, and an LCDR3 having an amino acid sequence of SEQ ID NOs:621, 622, and 623, respectively;
c. a VH comprising an HCDR1, and HCDR2 and an HCDR3 having an amino acid sequence of SEQ ID NOs:330, 331, and 332, respectively and a VL comprising an LCDR1, an LCDR2, and an LCDR3 having an amino acid sequence of SEQ ID NOs:333, 334, and 335, respectively;
d. a VH comprising an HCDR1, and HCDR2 and an HCDR3 having an amino acid sequence of SEQ ID NOs:936,937, and 938, respectively and a VL comprising an LCDR1, an LCDR2, and an LCDR3 having an amino acid sequence of SEQ ID NOs:939, 940, and 941, respectively;
e. a VH comprising an HCDR1, and HCDR2 and an HCDR3 having an amino acid sequence of SEQ ID NOs:312, 313, and 314, respectively and a VL comprising an LCDR1, an LCDR2, and an LCDR3 having an amino acid sequence of SEQ ID NOs:315, 316, and 317, respectively;
f. a VH comprising an HCDR1, and HCDR2 and an HCDR3 having an amino acid sequence of SEQ ID NOs:474, 475, and 476, respectively and a VL comprising an LCDR1, an LCDR2, and an LCDR3 having an amino acid sequence of SEQ ID NOs:477, 478, and 479, respectively;
g. a VH comprising an HCDR1, and HCDR2 and an HCDR3 having an amino acid sequence of SEQ ID NOs:1194, 1195, and 1196, respectively and a VL comprising an LCDR1, an LCDR2, and an LCDR3 having an amino acid sequence of SEQ ID NOs:1197, 1198, and 1199, respectively;
h. a VH comprising an HCDR1, and HCDR2 and an HCDR3 having an amino acid sequence of SEQ ID NOs:426, 427, and 428, respectively and a VL comprising an LCDR1, an LCDR2, and an LCDR3 having an amino acid sequence of SEQ ID NOs:429, 430, and 431, respectively; or
i. a VH comprising an HCDR1, and HCDR2 and an HCDR3 having an amino acid sequence of SEQ ID NOs:498, 499, and 500, respectively and a VL comprising an LCDR1, an LCDR2, and an LCDR3 having an amino acid sequence of SEQ ID NOs:501, 502, and 503, respectively.

13. The isolated monoclonal antibody or antigen binding fragment thereof of any of aspects 11 or 12 which recognizes human and rat IL-23R, wherein the antibody or fragment comprises:
a. a VH comprising the amino acid sequence of SEQ ID NO:202, and a VL comprising the amino acid sequence of SEQ ID NO:203;
b. a VH comprising the amino acid sequence of SEQ ID NO:252, and a VL comprising the amino acid sequence of SEQ ID NO:253;
c. a VH comprising the amino acid sequence of SEQ ID NO:156, and a VL comprising the amino acid sequence of SEQ ID NO:157;
d. a VH comprising the amino acid sequence of SEQ ID NO:798, and a VL comprising the amino acid sequence of SEQ ID NO:799;
e. a VH comprising the amino acid sequence of SEQ ID NO:150, and a VL comprising the amino acid sequence of SEQ ID NO:151;
f. a VH comprising the amino acid sequence of SEQ ID NO:204, and a VL comprising the amino acid sequence of SEQ ID NO:205;
g. a VH comprising the amino acid sequence of SEQ ID NO:884, and a VL comprising the amino acid sequence of SEQ ID NO:885;
h. a VH comprising the amino acid sequence of SEQ ID NO:188, and a VL comprising the amino acid sequence of SEQ ID NO:189; or
i. a VH comprising the amino acid sequence of SEQ ID NO:212, and a VL comprising the amino acid sequence of SEQ ID NO:213.

14. An isolated monoclonal antibody or antigen binding fragment thereof that binds interleukin-23 receptor (IL-23R) comprising a VL comprising CDR sequences of an LCDR1 SEQ ID NO: 1252, an LCDR2 SEQ ID NO:1253, and an LCDR3 SEQ ID NO:1254 and a VH comprising CDR sequences selected from:
a. an HCDR1 SEQ ID NO:900, an HCDR2 SEQ ID NO:901 and an HCDR3 SEQ ID NO:902;
b. an HCDR1 SEQ ID NO:906, an HCDR2 SEQ ID NO:907, and an HCDR3 SEQ ID NO:908;
c. an HCDR1 SEQ ID NO:912, an HCDR2 SEQ ID NO:913, and an HCDR3 SEQ ID NO:914;
d. an HCDR1 SEQ ID NO:918, an HCDR2 SEQ ID NO:919, and an HCDR3 SEQ ID NO:920;
e. an HCDR1 SEQ ID NO:942, an HCDR2 SEQ ID NO:943, and an HCDR3 SEQ ID NO:944;
f. an HCDR1 SEQ ID NO:948, an HCDR2 SEQ ID NO:949, and an HCDR3 SEQ ID NO:950;
g. an HCDR1 SEQ ID NO: 1014, an HCDR2 SEQ ID NO: 1015, and an HCDR3 SEQ ID NO:1016;
h. an HCDR1 SEQ ID NO: 1026, an HCDR2 SEQ ID NO: 1027, and an HCDR3 SEQ ID NO: 1028; and
i. an HCDR1 SEQ ID NO:1152, an HCDR2 SEQ ID NO:1153, and an HCDR3 SEQ ID NO:1154.

15. The isolated monoclonal antibody or antigen binding fragment thereof of aspect 13 comprising a VL comprising CDR sequences of an LCDR1 SEQ ID NO:1252, an LCDR2 SEQ ID NO: 1253, and an LCDR3 SEQ ID NO: 1254 or VL having the amino acid sequence SEQ ID NO: 1247 and a VH sequence selected from SEQ ID NO:786, 788, 790, 792, 800, 802, 824, 828 and 870.

16. An isolated monoclonal antibody or antigen binding fragment thereof that binds interleukin-23 receptor (IL-23R) comprising a VL comprising CDR sequences of an LCDR1 SEQ ID NO: 1255, an LCDR2 SEQ ID NO:1256, and an LCDR3 SEQ ID NO:1257 and a VH comprising CDR sequences selected from:
a. an HCDR1 SEQ ID NO:924, an HCDR2 SEQ ID NO:925, and an HCDR3 SEQ ID NO:926;
b. an HCDR1 SEQ ID NO: 1002, an HCDR2 SEQ ID NO: 1003, and an HCDR3 SEQ ID NO: 1004;
c. an HCDR1 SEQ ID NO:1032, an HCDR2 SEQ ID NO:1033, and an HCDR3 SEQ ID NO:1034;
d. an HCDR1 SEQ ID NO: 1044, an HCDR2 SEQ ID NO: 1045, and an HCDR3 SEQ ID NO:1046;
e. an HCDR1 SEQ ID NO:1050, an HCDR2 SEQ ID NO:1051, and an HCDR3 SEQ ID NO:1052;
f. an HCDR1 SEQ ID NO: 1056, an HCDR2 SEQ ID NO: 1057, and an HCDR3 SEQ ID NO:1058;
g. an HCDR1 SEQ ID NO: 1062, an HCDR2 SEQ ID NO: 1063, and an HCDR3 SEQ ID NO: 1064;
h. an HCDR1 SEQ ID NO: 1068, an HCDR2 SEQ ID NO: 1069, and an HCDR3 SEQ ID NO: 1070;
i. an HCDR1 SEQ ID NO:1080, an HCDR2 SEQ ID NO:1081, and an HCDR3 SEQ ID NO: 1082;
j. an HCDR1 SEQ ID NO:1092, an HCDR2 SEQ ID NO:1093, and an HCDR3 SEQ ID NO: 1094;
k. an HCDR1 SEQ ID NO: 1098, an HCDR2 SEQ ID NO: 1099, and an HCDR3 SEQ ID NO: 1100;
l. an HCDR1 SEQ ID NO:1110, an HCDR2 SEQ ID NO:1111, and an HCDR3 SEQ ID NO: 1112;
m. an HCDR1 SEQ ID NO:1122, an HCDR2 SEQ ID NO:1123, and an HCDR3 SEQ ID NO: 1124;
n. an HCDR1 SEQ ID NO:1140, an HCDR2 SEQ ID NO:1141, and an HCDR3 SEQ ID NO: 1142;
o. an HCDR1 SEQ ID NO: 1146, an HCDR2 SEQ ID NO: 1147, and an HCDR3 SEQ ID NO:1148; and
p. an HCDR1 SEQ ID NO: 1158, an HCDR2 SEQ ID NO: 1159, and an HCDR3 SEQ ID NO: 1160.

17. The isolated monoclonal antibody or antigen binding fragment thereof of aspect 16 comprising a VL comprising CDR sequences of an LCDR1 SEQ ID NO:1255, an LCDR2 SEQ ID NO: 1256, and an LCDR3 SEQ ID NO: 1257 or having the amino acid sequence SEQ ID NO:1248 and a VH sequence selected from SEQ ID NO:794, 820, 830, 834, 836, 838, 840, 842, 846, 850, 852, 856, 860, 866, 868 and 872.

18. An isolated monoclonal antibody or antigen binding fragment thereof that binds interleukin-23 receptor (IL-23R) comprising a VL comprising CDR sequences of an LCDR1 SEQ ID NO: 1258, an LCDR2 SEQ ID NO:1259, and an LCDR3 SEQ ID NO:1260 and a VH comprising CDR sequences selected from:
a. an HCDR1 SEQ ID NO:930, an HCDR2 SEQ ID NO:931, and an HCDR3 SEQ ID NO:932; and
b. an HCDR1 SEQ ID NO:954, an HCDR2 SEQ ID NO:955, and an HCDR3 SEQ ID NO:956.

19. The isolated monoclonal antibody or antigen binding fragment thereof of aspect 18 comprising a VL comprising CDR sequences of an LCDR1 SEQ ID NO:1258, an LCDR2 SEQ ID NO:1259, and an LCDR3 SEQ ID NO: 1260 or VL having the amino acid sequence SEQ ID NO:1249; and a VH sequence selected from SEQ ID NO:796 and 804.

20. An isolated monoclonal antibody or antigen binding fragment thereof that binds interleukin-23 receptor (IL-23R) comprising a VL comprising CDR sequences of an LCDR1 SEQ ID NO: 1261, an LCDR2 SEQ ID NO:1262, and an LCDR3 SEQ ID NO:1263 and a VH comprising CDR sequences selected from:
a. an HCDR1 SEQ ID NO:960, an HCDR2 SEQ ID NO:961, and an HCDR3 SEQ ID NO:962; and
b. an HCDR1 SEQ ID NO:1182, an HCDR2 SEQ ID NO:1183, and an HCDR3 SEQ ID NO:1184.

21. The isolated monoclonal antibody or antigen binding fragment thereof of aspect 20 comprising a VL comprising CDR sequences of an LCDR1 SEQ ID NO:1261, an LCDR2 SEQ ID NO: 1262, and an LCDR3 SEQ ID NO: 1263 or having the amino acid sequence SEQ ID NO:1250 and
a VH sequence selected from SEQ ID NO:806 and 880.

22. An isolated monoclonal antibody or antigen binding fragment thereof that binds interleukin-23 receptor (IL-23R) comprising a VL comprising CDR sequences of an LCDR1 SEQ ID NO: 1264, an LCDR2 SEQ ID NO:1265, and an LCDR3 SEQ ID NO:1266 and a VH comprising CDR sequences selected from:
a. an HCDR1 SEQ ID NO:1008, an HCDR2 SEQ ID NO:1009, and an HCDR3 SEQ ID NO:1010;
b. an HCDR1 SEQ ID NO: 1020, an HCDR2 SEQ ID NO:1021, and an HCDR3 SEQ ID NO: 1022;
c. an HCDR1 SEQ ID NO:1038, an HCDR2 SEQ ID NO:1039, and an HCDR3 SEQ ID NO: 1040;
d. an HCDR1 SEQ ID NO: 1074, an HCDR2 SEQ ID NO: 1075, and an HCDR3 SEQ ID NO: 1076;
e. an HCDR1 SEQ ID NO:1086, an HCDR2 SEQ ID NO:1087, and an HCDR3 SEQ ID NO: 1088;
f. an HCDR1 SEQ ID NO: 1104, an HCDR2 SEQ ID NO: 1105, and an HCDR3 SEQ ID NO:1106;
g. an HCDR1 SEQ ID NO: 1128, an HCDR2 SEQ ID NO: 1129, and an HCDR3 SEQ ID NO:1130; and
h. an HCDR1 SEQ ID NO:1134, an HCDR2 SEQ ID NO:1135, and an HCDR3 SEQ ID NO:1136.

23. The isolated monoclonal antibody or antigen binding fragment thereof of aspect 22 comprising a VL comprising CDR sequences of an LCDR1 SEQ ID NO:1264, an LCDR2 SEQ ID NO:1265, and an LCDR3 SEQ ID NO: 1266 or VL having the amino acid sequence SEQ ID NO:1251; and a VH sequence selected from SEQ ID NO: 806 and 880 822, 826, 832, 844, 848, 854, 862 and 864.

24. A VHH antibody or nanobody that binds interleukin-23 receptor (IL-23R) comprising a VH sequence selected from SEQ ID NO:786, 788, 790, 792, 800, 802, 824, 828, 870, 794, 820, 830, 834, 836, 838, 840, 842, 846, 850, 852, 856, 860, 866, 868, 872, 796, 804, 806, 880, 822, 826, 832, 844, 848, 854, 862 and 864.

25. An isolated monoclonal antibody or antigen binding fragment thereof that binds interleukin-23 receptor (IL-23R) comprising:
a. an HCDR1, an HCDR2, and an HCDR3 as set forth in SEQ ID NO: 138, and an LCDR1, an LCDR2, and an LCDR3 as set forth in SEQ ID NO:139;
b. an HCDR1, an HCDR2, and an HCDR3 as set forth in SEQ ID NO: 142, and an LCDR1, an LCDR2, and an LCDR3 as set forth in SEQ ID NO:143;
c. an HCDR1, an HCDR2, and an HCDR3 as set forth in SEQ ID NO: 144, and an LCDR1, an LCDR2, and an LCDR3 as set forth in SEQ ID NO:145;
d. an HCDR1, an HCDR2, and an HCDR3 as set forth in SEQ ID NO: 148, and an LCDR1, an LCDR2, and an LCDR3 as set forth in SEQ ID NO:149;
e. an HCDR1, an HCDR2, and an HCDR3 as set forth in SEQ ID NO: 150, and an LCDR1, an LCDR2, and an LCDR3 as set forth in SEQ ID NO:151;
f. an HCDR1, an HCDR2, and an HCDR3 as set forth in SEQ ID NO: 152, and an LCDR1, an LCDR2, and an LCDR3 as set forth in SEQ ID NO:153;
g. an HCDR1, an HCDR2, and an HCDR3 as set forth in SEQ ID NO: 154, and an LCDR1, an LCDR2, and an LCDR3 as set forth in SEQ ID NO:155;
h. an HCDR1, an HCDR2, and an HCDR3 as set forth in SEQ ID NO: 156, and an LCDR1, an LCDR2, and an LCDR3 as set forth in SEQ ID NO:157;
i. an HCDR1, an HCDR2, and an HCDR3 as set forth in SEQ ID NO: 158, and an LCDR1, an LCDR2, and an LCDR3 as set forth in SEQ ID NO:159;
j. an HCDR1, an HCDR2, and an HCDR3 as set forth in SEQ ID NO: 160, and an LCDR1, an LCDR2, and an LCDR3 as set forth in SEQ ID NO:161;
k. an HCDR1, an HCDR2, and an HCDR3 as set forth in SEQ ID NO: 162, and an LCDR1, an LCDR2, and an LCDR3 as set forth in SEQ ID NO:163;
l. an HCDR1, an HCDR2, and an HCDR3 as set forth in SEQ ID NO: 164, and an LCDR1, an LCDR2, and an LCDR3 as set forth in SEQ ID NO:165;
m. an HCDR1, an HCDR2, and an HCDR3 as set forth in SEQ ID NO: 166, and an LCDR1, an LCDR2, and an LCDR3 as set forth in SEQ ID NO:167;
n. an HCDR1, an HCDR2, and an HCDR3 as set forth in SEQ ID NO: 168, and an LCDR1, an LCDR2, and an LCDR3 as set forth in SEQ ID NO:169;
o. an HCDR1, an HCDR2, and an HCDR3 as set forth in SEQ ID NO: 170, and an LCDR1, an LCDR2, and an LCDR3 as set forth in SEQ ID NO:171;
p. an HCDR1, an HCDR2, and an HCDR3 as set forth in SEQ ID NO: 172, and an LCDR1, an LCDR2, and an LCDR3 as set forth in SEQ ID NO:173;
q. an HCDR1, an HCDR2, and an HCDR3 as set forth in SEQ ID NO: 174, and an LCDR1, an LCDR2, and an LCDR3 as set forth in SEQ ID NO:175;
r. an HCDR1, an HCDR2, and an HCDR3 as set forth in SEQ ID NO: 176, and an LCDR1, an LCDR2, and an LCDR3 as set forth in SEQ ID NO:177;
s. an HCDR1, an HCDR2, and an HCDR3 as set forth in SEQ ID NO: 178, and an LCDR1, an LCDR2, and an LCDR3 as set forth in SEQ ID NO:179;
t. an HCDR1, an HCDR2, and an HCDR3 as set forth in SEQ ID NO: 180, and an LCDR1, an LCDR2, and an LCDR3 as set forth in SEQ ID NO:181;
u. an HCDR1, an HCDR2, and an HCDR3 as set forth in SEQ ID NO: 182, and an LCDR1, an LCDR2, and an LCDR3 as set forth in SEQ ID NO:183;
v. an HCDR1, an HCDR2, and an HCDR3 as set forth in SEQ ID NO: 186, and an LCDR1, an LCDR2, and an LCDR3 as set forth in SEQ ID NO:187;
w. an HCDR1, an HCDR2, and an HCDR3 as set forth in SEQ ID NO: 188, and an LCDR1, an LCDR2, and an LCDR3 as set forth in SEQ ID NO:189;
x. an HCDR1, an HCDR2, and an HCDR3 as set forth in SEQ ID NO: 190, and an LCDR1, an LCDR2, and an LCDR3 as set forth in SEQ ID NO:191;
y. an HCDR1, an HCDR2, and an HCDR3 as set forth in SEQ ID NO: 192, and an LCDR1, an LCDR2, and an LCDR3 as set forth in SEQ ID NO:193;
z. an HCDR1, an HCDR2, and an HCDR3 as set forth in SEQ ID NO: 194, and an LCDR1, an LCDR2, and an LCDR3 as set forth in SEQ ID NO:195;
aa. an HCDR1, an HCDR2, and an HCDR3 as set forth in SEQ ID NO: 196, and an LCDR1, an LCDR2, and an LCDR3 as set forth in SEQ ID NO:197;
bb. an HCDR1, an HCDR2, and an HCDR3 as set forth in SEQ ID NO: 198, and an LCDR1, an LCDR2, and an LCDR3 as set forth in SEQ ID NO:199;
cc. an HCDR1, an HCDR2, and an HCDR3 as set forth in SEQ ID NO:200, and an LCDR1, an LCDR2, and an LCDR3 as set forth in SEQ ID NO:201;
dd. an HCDR1, an HCDR2, and an HCDR3 as set forth in SEQ ID NO:202, and an LCDR1, an LCDR2, and an LCDR3 as set forth in SEQ ID NO:203;
ee. an HCDR1, an HCDR2, and an HCDR3 as set forth in SEQ ID NO:204, and an LCDR1, an LCDR2, and an LCDR3 as set forth in SEQ ID NO:205;
ff. an HCDR1, an HCDR2, and an HCDR3 as set forth in SEQ ID NO:206, and an LCDR1, an LCDR2, and an LCDR3 as set forth in SEQ ID NO:207;
gg. an HCDR1, an HCDR2, and an HCDR3 as set forth in SEQ ID NO:208, and an LCDR1, an LCDR2, and an LCDR3 as set forth in SEQ ID NO:209;
hh. an HCDR1, an HCDR2, and an HCDR3 as set forth in SEQ ID NO:210, and an LCDR1, an LCDR2, and an LCDR3 as set forth in SEQ ID NO:211;
ii. an HCDR1, an HCDR2, and an HCDR3 as set forth in SEQ ID NO:212, and an LCDR1, an LCDR2, and an LCDR3 as set forth in SEQ ID NO:213;
jj. an HCDR1, an HCDR2, and an HCDR3 as set forth in SEQ ID NO:214, and an LCDR1, an LCDR2, and an LCDR3 as set forth in SEQ ID NO:215;
kk. an HCDR1, an HCDR2, and an HCDR3 as set forth in SEQ ID NO:216, and an LCDR1, an LCDR2, and an LCDR3 as set forth in SEQ ID NO:217;
ll. an HCDR1, an HCDR2, and an HCDR3 as set forth in SEQ ID NO:218, and an LCDR1, an LCDR2, and an LCDR3 as set forth in SEQ ID NO:219;
mm. an HCDR1, an HCDR2, and an HCDR3 as set forth in SEQ ID NO:220, and an LCDR1, an LCDR2, and an LCDR3 as set forth in SEQ ID NO:221;
nn. an HCDR1, an HCDR2, and an HCDR3 as set forth in SEQ ID NO:222, and an LCDR1, an LCDR2, and an LCDR3 as set forth in SEQ ID NO:223;
oo. an HCDR1, an HCDR2, and an HCDR3 as set forth in SEQ ID NO:226, and an LCDR1, an LCDR2, and an LCDR3 as set forth in SEQ ID NO:227;
pp. an HCDR1, an HCDR2, and an HCDR3 as set forth in SEQ ID NO:228, and an LCDR1, an LCDR2, and an LCDR3 as set forth in SEQ ID NO:229;
qq. an HCDR1, an HCDR2, and an HCDR3 as set forth in SEQ ID NO:230, and an LCDR1, an LCDR2, and an LCDR3 as set forth in SEQ ID NO:231;
rr. an HCDR1, an HCDR2, and an HCDR3 as set forth in SEQ ID NO:232, and an LCDR1, an LCDR2, and an LCDR3 as set forth in SEQ ID NO:233;
ss. an HCDR1, an HCDR2, and an HCDR3 as set forth in SEQ ID NO:234, and an LCDR1, an LCDR2, and an LCDR3 as set forth in SEQ ID NO:235;
tt. an HCDR1, an HCDR2, and an HCDR3 as set forth in SEQ ID NO:236, and an LCDR1, an LCDR2, and an LCDR3 as set forth in SEQ ID NO:237;
uu. an HCDR1, an HCDR2, and an HCDR3 as set forth in SEQ ID NO:238, and an LCDR1, an LCDR2, and an LCDR3 as set forth in SEQ ID NO:239;
vv. an HCDR1, an HCDR2, and an HCDR3 as set forth in SEQ ID NO:240, and an LCDR1, an LCDR2, and an LCDR3 as set forth in SEQ ID NO:241;
ww. an HCDR1, an HCDR2, and an HCDR3 as set forth in SEQ ID NO:246, and an LCDR1, an LCDR2, and an LCDR3 as set forth in SEQ ID NO:247;
xx. an HCDR1, an HCDR2, and an HCDR3 as set forth in SEQ ID NO:248, and an LCDR1, an LCDR2, and an LCDR3 as set forth in SEQ ID NO:249;
yy. an HCDR1, an HCDR2, and an HCDR3 as set forth in SEQ ID NO:250, and an LCDR1, an LCDR2, and an LCDR3 as set forth in SEQ ID NO:251;
zz. an HCDR1, an HCDR2, and an HCDR3 as set forth in SEQ ID NO:252, and an LCDR1, an LCDR2, and an LCDR3 as set forth in SEQ ID NO:253;
aaa. an HCDR1, an HCDR2, and an HCDR3 as set forth in SEQ ID NO:254, and an LCDR1, an LCDR2, and an LCDR3 as set forth in SEQ ID NO:255;
bbb. an HCDR1, an HCDR2, and an HCDR3 as set forth in SEQ ID NO:256, and an LCDR1, an LCDR2, and an LCDR3 as set forth in SEQ ID NO:257;
ccc. an HCDR1, an HCDR2, and an HCDR3 as set forth in SEQ ID NO:258, and an LCDR1, an LCDR2, and an LCDR3 as set forth in SEQ ID NO:259;
ddd. an HCDR1, an HCDR2, and an HCDR3 as set forth in SEQ ID NO:260, and an LCDR1, an LCDR2, and an LCDR3 as set forth in SEQ ID NO:261;
eee. an HCDR1, an HCDR2, and an HCDR3 as set forth in SEQ ID NO:262, and a LCDR1, an LCDR2, and an LCDR3 as set forth in SEQ ID NO:263;
fff. an HCDR1, an HCDR2, and an HCDR3 as set forth in SEQ ID NO:264, and an LCDR1, an LCDR2, and an LCDR3 as set forth in SEQ ID NO:265;
ggg. an HCDR1, an HCDR2, and an HCDR3 as set forth in SEQ ID NO:268, and an LCDR1, an LCDR2, and an LCDR3 as set forth in SEQ ID NO:269;
hhh. an HCDR1, an HCDR2, and an HCDR3 as set forth in SEQ ID NO:786, and an LCDR1, an LCDR2, and an LCDR3 as set forth in SEQ ID NO:787;
iii. an HCDR1, an HCDR2, and an HCDR3 as set forth in SEQ ID NO:788, and an LCDR1, an LCDR2, and an LCDR3 as set forth in SEQ ID NO:789;
jjj. an HCDR1, an HCDR2, and an HCDR3 as set forth in SEQ ID NO:790, and an LCDR1, an LCDR2, and an LCDR3 as set forth in SEQ ID NO:791;
kkk. an HCDR1, an HCDR2, and an HCDR3 as set forth in SEQ ID NO:792, and an LCDR1, an LCDR2, and an LCDR3 as set forth in SEQ ID NO:793;
lll. an HCDR1, an HCDR2, and an HCDR3 as set forth in SEQ ID NO:798, and an LCDR1, an LCDR2, and an LCDR3 as set forth in SEQ ID NO:799;
mmm. an HCDR1, an HCDR2, and an HCDR3 as set forth in SEQ ID NO:806, and an LCDR1, an LCDR2, and an LCDR3 as set forth in SEQ ID NO:807;
nnn. an HCDR1, an HCDR2, and an HCDR3 as set forth in SEQ ID NO:808, and an LCDR1, an LCDR2, and an LCDR3 as set forth in SEQ ID NO:809;
ooo. an HCDR1, an HCDR2, and an HCDR3 as set forth in SEQ ID NO:810, and an LCDR1, an LCDR2, and an LCDR3 as set forth in SEQ ID NO:811;
ppp. an HCDR1, an HCDR2, and an HCDR3 as set forth in SEQ ID NO:812, and an LCDR1, an LCDR2, and an LCDR3 as set forth in SEQ ID NO:813;
qqq. an HCDR1, an HCDR2, and an HCDR3 as set forth in SEQ ID NO:814, and an LCDR1, an LCDR2, and an LCDR3 as set forth in SEQ ID NO:815;
rrr. an HCDR1, an HCDR2, and an HCDR3 as set forth in SEQ ID NO:816, and an LCDR1, an LCDR2, and an LCDR3 as set forth in SEQ ID NO:817;
sss. an HCDR1, an HCDR2, and an HCDR3 as set forth in SEQ ID NO:818, and an LCDR1, an LCDR2, and an LCDR3 as set forth in SEQ ID NO:819;
ttt. an HCDR1, an HCDR2, and an HCDR3 as set forth in SEQ ID NO:820, and an LCDR1, an LCDR2, and an LCDR3 as set forth in SEQ ID NO:821;
uuu. an HCDR1, an HCDR2, and an HCDR3 as set forth in SEQ ID NO:822, and an LCDR1, an LCDR2, and an LCDR3 as set forth in SEQ ID NO:823;
vvv. an HCDR1, an HCDR2, and an HCDR3 as set forth in SEQ ID NO:824, and an LCDR1, an LCDR2, and an LCDR3 as set forth in SEQ ID NO:825;
www.an HCDR1, an HCDR2, and an HCDR3 as set forth in SEQ ID NO:826, and an LCDR1, an LCDR2, and an LCDR3 as set forth in SEQ ID NO:827;
xxx. an HCDR1, an HCDR2, and an HCDR3 as set forth in SEQ ID NO:828, and an LCDR1, an LCDR2, and an LCDR3 as set forth in SEQ ID NO:829;
yyy. an HCDR1, an HCDR2, and an HCDR3 as set forth in SEQ ID NO:830, and an LCDR1, an LCDR2, and an LCDR3 as set forth in SEQ ID NO:831;
zzz. an HCDR1, an HCDR2, and an HCDR3 as set forth in SEQ ID NO:832, and an LCDR1, an LCDR2, and an LCDR3 as set forth in SEQ ID NO:833;
aaaa. an HCDR1, an HCDR2, and an HCDR3 as set forth in SEQ ID NO:836, and an LCDR1, an LCDR2, and an LCDR3 as set forth in SEQ ID NO:837;
bbbb. an HCDR1, an HCDR2, and an HCDR3 as set forth in SEQ ID NO:838, and an LCDR1, an LCDR2, and an LCDR3 as set forth in SEQ ID NO:839;
cccc. an HCDR1, an HCDR2, and an HCDR3 as set forth in SEQ ID NO:840, and an LCDR1, an LCDR2, and an LCDR3 as set forth in SEQ ID NO:841;
dddd. an HCDR1, an HCDR2, and an HCDR3 as set forth in SEQ ID NO: 842, and an LCDR1, an LCDR2, and an LCDR3 as set forth in SEQ ID NO:843;
eeee. an HCDR1, an HCDR2, and an HCDR3 as set forth in SEQ ID NO:846, and an LCDR1, an LCDR2, and an LCDR3 as set forth in SEQ ID NO:847;
ffff. an HCDR1, an HCDR2, and an HCDR3 as set forth in SEQ ID NO:848, and an LCDR1, an LCDR2, and an LCDR3 as set forth in SEQ ID NO:849;
gggg. an HCDR1, an HCDR2, and an HCDR3 as set forth in SEQ ID NO:850, and an LCDR1, an LCDR2, and an LCDR3 as set forth in SEQ ID NO:851;
hhhh. an HCDR1, an HCDR2, and an HCDR3 as set forth in SEQ ID NO:852, and an LCDR1, an LCDR2, and an LCDR3 as set forth in SEQ ID NO:853;
iiii. an HCDR1, an HCDR2, and an HCDR3 as set forth in SEQ ID NO:854, and an LCDR1, an LCDR2, and an LCDR3 as set forth in SEQ ID NO:855;
jjjj. an HCDR1, an HCDR2, and an HCDR3 as set forth in SEQ ID NO:856, and an LCDR1, an LCDR2, and an LCDR3 as set forth in SEQ ID NO:857;
kkkk. an HCDR1, an HCDR2, and an HCDR3 as set forth in SEQ ID NO:858, and an LCDR1, an LCDR2, and an LCDR3 as set forth in SEQ ID NO:859;
llll. an HCDR1, an HCDR2, and an HCDR3 as set forth in SEQ ID NO:860, and an LCDR1, an LCDR2, and an LCDR3 as set forth in SEQ ID NO:861;
mmmm. an HCDR1, an HCDR2, and an HCDR3 as set forth in SEQ ID NO:862, and an LCDR1, an LCDR2, and an LCDR3 as set forth in SEQ ID NO:863;
nnnn. an HCDR1, an HCDR2, and an HCDR3 as set forth in SEQ ID NO:864, and an LCDR1, an LCDR2, and an LCDR3 as set forth in SEQ ID NO:865;
oooo. an HCDR1, an HCDR2, and an HCDR3 as set forth in SEQ ID NO:866, and an LCDR1, an LCDR2, and an LCDR3 as set forth in SEQ ID NO:867;
pppp. an HCDR1, an HCDR2, and an HCDR3 as set forth in SEQ ID NO:868, and an LCDR1, an LCDR2, and an LCDR3 as set forth in SEQ ID NO:869;
qqqq. an HCDR1, an HCDR2, and an HCDR3 as set forth in SEQ ID NO:870, and an LCDR1, an LCDR2, and an LCDR3 as set forth in SEQ ID NO:871;
rrrr. an HCDR1, an HCDR2, and an HCDR3 as set forth in SEQ ID NO:872, and an LCDR1, an LCDR2, and an LCDR3 as set forth in SEQ ID NO:873;
ssss. an HCDR1, an HCDR2, and an HCDR3 as set forth in SEQ ID NO:874, and an LCDR1, an LCDR2, and an LCDR3 as set forth in SEQ ID NO:875;
tttt. an HCDR1, an HCDR2, and an HCDR3 as set forth in SEQ ID NO:876, and an LCDR1, an LCDR2, and an LCDR3 as set forth in SEQ ID NO:877;
uuuu. an HCDR1, an HCDR2, and an HCDR3 as set forth in SEQ ID NO:878, and an LCDR1, an LCDR2, and an LCDR3 as set forth in SEQ ID NO:879;
vvvv. an HCDR1, an HCDR2, and an HCDR3 as set forth in SEQ ID NO:880, and an LCDR1, an LCDR2, and an LCDR3 as set forth in SEQ ID NO:881;
wwww. an HCDR1, an HCDR2, and an HCDR3 as set forth in SEQ ID NO:882, and an LCDR1, an LCDR2, and an LCDR3 as set forth in SEQ ID NO:883;
xxxx. an HCDR1, an HCDR2, and an HCDR3 as set forth in SEQ ID NO:884, and an LCDR1, an LCDR2, and an LCDR3 as set forth in SEQ ID NO:885;
yyyy. an HCDR1, an HCDR2, and an HCDR3 as set forth in SEQ ID NO:886, and an LCDR1, an LCDR2, and an LCDR3 as set forth in SEQ ID NO:887;
zzzz. an HCDR1, an HCDR2, and an HCDR3 as set forth in SEQ ID NO:888, and an LCDR1, an LCDR2, and an LCDR3 as set forth in SEQ ID NO:889; or
aaaaa. an HCDR1, an HCDR2, and an HCDR3 as set forth in SEQ ID NO:890, and an LCDR1, an LCDR2, and an LCDR3 as set forth in SEQ ID NO:891.

26. An isolated monoclonal antibody or antigen binding fragment thereof that binds interleukin-23 receptor (IL-23R) comprising:
a. a VH comprising an HCDR1, an HCDR2, and an HCDR3 having an amino acid sequence of SEQ ID NOs:276, 277, and 278, respectively, and a VL comprising an LCDR1, an LCDR2, and an LCDR3 having an amino acid sequence of SEQ ID NOs:279, 280, and 281, respectively;
b. a VH comprising an HCDR1, an HCDR2, and an HCDR3 having an amino acid sequence of SEQ ID NOs:288, 289, and 290, respectively, and a VL comprising an LCDR1, an LCDR2, and an LCDR3 having an amino acid sequence of SEQ ID NOs:291, 292, and 293, respectively;
c. a VH comprising an HCDR1, an HCDR2, and an HCDR3 having an amino acid sequence of SEQ ID NOs:294, 295, and 296, respectively, and a VL comprising an LCDR1, an LCDR2, and an LCDR3 having an amino acid sequence of SEQ ID NOs:297, 298, and 299, respectively;
d. a VH comprising an HCDR1, an HCDR2, and an HCDR3 having an amino acid sequence of SEQ ID NOs:306, 307, and 308, respectively, and a VL comprising an LCDR1, an LCDR2, and an LCDR3 having an amino acid sequence of SEQ ID NOs:309, 310, and 311, respectively;
e. a VH comprising an HCDR1, an HCDR2, and an HCDR3 having an amino acid sequence of SEQ ID NOs:312, 313, and 314, respectively, and a VL comprising an LCDR1, an LCDR2, and an LCDR3 having an amino acid sequence of SEQ ID NOs:315, 316, and 317,
f. a VH comprising an HCDR1, an HCDR2, and an HCDR3 having an amino acid sequence of SEQ ID NOs:318, 319, and 320, respectively, and a VL comprising an LCDR1, an LCDR2, and an LCDR3 having an amino acid sequence of SEQ ID NOs:321, 322, and 323, respectively;
g. a VH comprising an HCDR1, an HCDR2, and an HCDR3 having an amino acid sequence of SEQ ID NOs:324, 325, and 326, respectively, and a VL comprising an LCDR1, an LCDR2, and an LCDR3 having an amino acid sequence of SEQ ID NOs:327, 328, and 329, respectively;
h. a VH comprising an HCDR1, an HCDR2, and an HCDR3 having an amino acid sequence of SEQ ID NOs:330, 331, and 332, respectively, and a VL comprising an LCDR1, an LCDR2, and an LCDR3 having an amino acid sequence of SEQ ID NOs:333, 334, and 335, respectively;
i. a VH comprising an HCDR1, an HCDR2, and an HCDR3 having an amino acid sequence of SEQ ID NOs:336, 337, and 338, respectively, and a VL comprising an LCDR1, an LCDR2, and an LCDR3 having an amino acid sequence of SEQ ID NOs:339, 340, and 341, respectively;
j. a VH comprising an HCDR1, an HCDR2, and an HCDR3 having an amino acid sequence of SEQ ID NOs:342, 343, and 344, respectively, and a VL comprising an LCDR1, an LCDR2, and an LCDR3 having an amino acid sequence of SEQ ID NOs:345, 346, and 347, respectively;
k. a VH comprising an HCDR1, an HCDR2, and an HCDR3 having an amino acid sequence of SEQ ID NOs:348, 349, and 350, respectively, and a VL comprising an LCDR1, an LCDR2, and an LCDR3 having an amino acid sequence of SEQ ID NOs:351, 352, and 353, respectively;
l. a VH comprising an HCDR1, an HCDR2, and an HCDR3 having an amino acid sequence of SEQ ID NOs:354, 355, and 356, respectively, and a VL comprising an LCDR1, an LCDR2, and an LCDR3 having an amino acid sequence of SEQ ID NOs:357, 358, and 359, respectively;
m. a VH comprising an HCDR1, an HCDR2, and an HCDR3 having an amino acid sequence of SEQ ID NOs:360, 361, and 362, respectively, and a VL comprising an LCDR1, an LCDR2, and an LCDR3 having an amino acid sequence of SEQ ID NOs:363, 364, and 365, respectively;
n. a VH comprising an HCDR1, an HCDR2, and an HCDR3 having an amino acid sequence of SEQ ID NOs:366, 367, and 368, respectively, and a VL comprising an LCDR1, an LCDR2, and an LCDR3 having an amino acid sequence of SEQ ID NOs:369, 370, and 371,
o. a VH comprising an HCDR1, an HCDR2, and an HCDR3 having an amino acid sequence of SEQ ID NOs:372, 373, and 374, respectively, and a VL comprising an LCDR1, an LCDR2, and an LCDR3 having an amino acid sequence of SEQ ID NOs:375, 376, and 377, respectively;
p. a VH comprising an HCDR1, an HCDR2, and an HCDR3 having an amino acid sequence of SEQ ID NOs:378, 379, and 380, respectively, and a VL comprising an LCDR1, an LCDR2, and an LCDR3 having an amino acid sequence of SEQ ID NOs:381, 382, and 383, respectively;
q. a VH comprising an HCDR1, an HCDR2, and an HCDR3 having an amino acid sequence of SEQ ID NOs:384, 385, and 386, respectively, and a VL comprising an LCDR1, an LCDR2, and an LCDR3 having an amino acid sequence of SEQ ID NOs:387, 388, and 389, respectively;
r. a VH comprising an HCDR1, an HCDR2, and an HCDR3 having an amino acid sequence of SEQ ID NOs:390, 391, and 392, respectively, and a VL comprising an LCDR1, an LCDR2, and an LCDR3 having an amino acid sequence of SEQ ID NOs:393, 394, and 395, respectively;
s. a VH comprising an HCDR1, an HCDR2, and an HCDR3 having an amino acid sequence of SEQ ID NOs:396, 397, and 398, respectively, and a VL comprising an LCDR1, an LCDR2, and an LCDR3 having an amino acid sequence of SEQ ID NOs:399, 400, and 401, respectively;
t. a VH comprising an HCDR1, an HCDR2, and an HCDR3 having an amino acid sequence of SEQ ID NOs:402, 403, and 404, respectively, and a VL comprising an LCDR1, an LCDR2, and an LCDR3 having an amino acid sequence of SEQ ID NOs:405, 406, and 407, respectively;
u. a VH comprising an HCDR1, an HCDR2, and an HCDR3 having an amino acid sequence of SEQ ID NOs:408, 409, and 410, respectively, and a VL comprising an LCDR1, an LCDR2, and an LCDR3 having an amino acid sequence of SEQ ID NOs:411, 412, and 413, respectively;
v. a VH comprising an HCDR1, an HCDR2, and an HCDR3 having an amino acid sequence of SEQ ID NOs:420, 421, and 422, respectively, and a VL comprising an LCDR1, an LCDR2, and an LCDR3 having an amino acid sequence of SEQ ID NOs:423, 424, and 425, respectively;
w. a VH comprising an HCDR1, an HCDR2, and an HCDR3 having an amino acid sequence of SEQ ID NOs:426, 427, and 428, respectively, and a VL comprising an LCDR1, an LCDR2, and an LCDR3 having an amino acid sequence of SEQ ID NOs:429, 430, and 431,
x. a VH comprising an HCDR1, an HCDR2, and an HCDR3 having an amino acid sequence of SEQ ID NOs:432, 433, and 434, respectively, and a VL comprising an LCDR1, an LCDR2, and an LCDR3 having an amino acid sequence of SEQ ID NOs:435, 436, and 437, respectively;
y. a VH comprising an HCDR1, an HCDR2, and an HCDR3 having an amino acid sequence of SEQ ID NOs:438, 439, and 440, respectively, and a VL comprising an LCDR1, an LCDR2, and an LCDR3 having an amino acid sequence of SEQ ID NOs:441, 442, and 443, respectively;
z. a VH comprising an HCDR1, an HCDR2, and an HCDR3 having an amino acid sequence of SEQ ID NOs:444, 445, and 446, respectively, and a VL comprising an LCDR1, an LCDR2, and an LCDR3 having an amino acid sequence of SEQ ID NOs:447, 448, and 449, respectively;
aa. a VH comprising an HCDR1, an HCDR2, and an HCDR3 having an amino acid sequence of SEQ ID NOs:450, 451, and 452, respectively, and a VL comprising an LCDR1, an LCDR2, and an LCDR3 having an amino acid sequence of SEQ ID NOs:453, 454, and 455, respectively;
bb. a VH comprising an HCDR1, an HCDR2, and an HCDR3 having an amino acid sequence of SEQ ID NOs:456, 457, and 458, respectively, and a VL comprising an LCDR1, an LCDR2, and an LCDR3 having an amino acid sequence of SEQ ID NOs:459, 460, and 461, respectively;
cc. a VH comprising an HCDR1, an HCDR2, and an HCDR3 having an amino acid sequence of SEQ ID NOs:462, 463, and 464, respectively, and a VL comprising an LCDR1, an LCDR2, and an LCDR3 having an amino acid sequence of SEQ ID NOs:465, 466, and 467, respectively;
dd. a VH comprising an HCDR1, an HCDR2, and an HCDR3 having an amino acid sequence of SEQ ID NOs:468, 469, and 470, respectively, and a VL comprising an LCDR1, an LCDR2, and an LCDR3 having an amino acid sequence of SEQ ID NOs:471, 472, and 473, respectively;
ee. a VH comprising an HCDR1, an HCDR2, and an HCDR3 having an amino acid sequence of SEQ ID NOs:474, 475, and 476, respectively, and a VL comprising an LCDR1, an LCDR2, and an LCDR3 having an amino acid sequence of SEQ ID NOs:477, 478, and 479, respectively;
ff. a VH comprising an HCDR1, an HCDR2, and an HCDR3 having an amino acid sequence of SEQ ID NOs:480, 481, and 482, respectively, and a VL comprising an LCDR1, an LCDR2, and an LCDR3 having an amino acid sequence of SEQ ID NOs:483, 484, and 485,
gg. a VH comprising an HCDR1, an HCDR2, and an HCDR3 having an amino acid sequence of SEQ ID NOs:486, 487, and 488, respectively, and a VL comprising an LCDR1, an LCDR2, and an LCDR3 having an amino acid sequence of SEQ ID NOs:489, 490, and 491, respectively;
hh. a VH comprising an HCDR1, an HCDR2, and an HCDR3 having an amino acid sequence of SEQ ID NOs:492, 493, and 494, respectively, and a VL comprising an LCDR1, an LCDR2, and an LCDR3 having an amino acid sequence of SEQ ID NOs:495, 496, and 497, respectively;
ii. a VH comprising an HCDR1, an HCDR2, and an HCDR3 having an amino acid sequence of SEQ ID NOs:498, 499, and 500, respectively, and a VL comprising an LCDR1, an LCDR2, and an LCDR3 having an amino acid sequence of SEQ ID NOs:501, 502, and 503, respectively;
jj. a VH comprising an HCDR1, an HCDR2, and an HCDR3 having an amino acid sequence of SEQ ID NOs:504, 505, and 506, respectively, and a VL comprising an LCDR1, an LCDR2, and an LCDR3 having an amino acid sequence of SEQ ID NOs:507, 508, and 509, respectively;
kk. a VH comprising an HCDR1, an HCDR2, and an HCDR3 having an amino acid sequence of SEQ ID NOs:510, 511, and 512, respectively, and a VL comprising an LCDR1, an LCDR2, and an LCDR3 having an amino acid sequence of SEQ ID NOs:513, 514, and 515, respectively;
ll. a VH comprising an HCDR1, an HCDR2, and an HCDR3 having an amino acid sequence of SEQ ID NOs:516, 517, and 518, respectively, and a VL comprising an LCDR1, an LCDR2, and an LCDR3 having an amino acid sequence of SEQ ID NOs:519, 520, and 521, respectively;
mm. a VH comprising an HCDR1, an HCDR2, and an HCDR3 having an amino acid sequence of SEQ ID NOs:522, 523, and 524, respectively, and a VL comprising an LCDR1, an LCDR2, and an LCDR3 having an amino acid sequence of SEQ ID NOs:525, 526, and 527, respectively;
nn. a VH comprising an HCDR1, an HCDR2, and an HCDR3 having an amino acid sequence of SEQ ID NOs:528, 529, and 530, respectively, and a VL comprising an LCDR1, an LCDR2, and an LCDR3 having an amino acid sequence of SEQ ID NOs:531, 532, and 533, respectively;
oo. a VH comprising an HCDR1, an HCDR2, and an HCDR3 having an amino acid sequence of SEQ ID NOs:540, 541, and 542, respectively, and a VL comprising an LCDR1, an LCDR2, and an LCDR3 having an amino acid sequence of SEQ ID NOs:543, 544, and 545,
pp. a VH comprising an HCDR1, an HCDR2, and an HCDR3 having an amino acid sequence of SEQ ID NOs:546, 547, and 548, respectively, and a VL comprising an LCDR1, an LCDR2, and an LCDR3 having an amino acid sequence of SEQ ID NOs:549, 550, and 551, respectively;
qq. a VH comprising an HCDR1, an HCDR2, and an HCDR3 having an amino acid sequence of SEQ ID NOs:552, 553, and 554, respectively, and a VL comprising an LCDR1, an LCDR2, and an LCDR3 having an amino acid sequence of SEQ ID NOs:555, 556, and 557, respectively;
rr. a VH comprising an HCDR1, an HCDR2, and an HCDR3 having an amino acid sequence of SEQ ID NOs:558, 559, and 560, respectively, and a VL comprising an LCDR1, an LCDR2, and an LCDR3 having an amino acid sequence of SEQ ID NOs:561, 562, and 563, respectively;
ss. a VH comprising an HCDR1, an HCDR2, and an HCDR3 having an amino acid sequence of SEQ ID NOs:564, 565, and 566, respectively, and a VL comprising an LCDR1, an LCDR2, and an LCDR3 having an amino acid sequence of SEQ ID NOs:567, 568, and 569, respectively;
tt. a VH comprising an HCDR1, an HCDR2, and an HCDR3 having an amino acid sequence of SEQ ID NOs:570, 571, and 572, respectively, and a VL comprising an LCDR1, an LCDR2, and an LCDR3 having an amino acid sequence of SEQ ID NOs:573, 574, and 575, respectively;
uu. a VH comprising an HCDR1, an HCDR2, and an HCDR3 having an amino acid sequence of SEQ ID NOs:576, 577, and 578, respectively, and a VL comprising an LCDR1, an LCDR2, and an LCDR3 having an amino acid sequence of SEQ ID NOs:579, 580, and 581, respectively;
vv. a VH comprising an HCDR1, an HCDR2, and an HCDR3 having an amino acid sequence of SEQ ID NOs:582, 583, and 584, respectively, and a VL comprising an LCDR1, an LCDR2, and an LCDR3 having an amino acid sequence of SEQ ID NOs:585, 586, and 587, respectively;
ww. a VH comprising an HCDR1, an HCDR2, and an HCDR3 having an amino acid sequence of SEQ ID NOs:600, 601, and 602, respectively, and a VL comprising an LCDR1, an LCDR2, and an LCDR3 having an amino acid sequence of SEQ ID NOs:603, 604, and 605, respectively;
xx. a VH comprising an HCDR1, an HCDR2, and an HCDR3 having an amino acid sequence of SEQ ID NOs:606, 607, and 608, respectively, and a VL comprising an LCDR1, an LCDR2, and an LCDR3 having an amino acid sequence of SEQ ID NOs:609, 610, and 611,
yy. a VH comprising an HCDR1, an HCDR2, and an HCDR3 having an amino acid sequence of SEQ ID NOs:612, 613, and 614, respectively, and a VL comprising an LCDR1, an LCDR2, and an LCDR3 having an amino acid sequence of SEQ ID NOs:615, 616, and 617, respectively;
zz. a VH comprising an HCDR1, an HCDR2, and an HCDR3 having an amino acid sequence of SEQ ID NOs:618, 619, and 620, respectively, and a VL comprising an LCDR1, an LCDR2, and an LCDR3 having an amino acid sequence of SEQ ID NOs:621, 622, and 623, respectively;
aaa. a VH comprising an HCDR1, an HCDR2, and an HCDR3 having an amino acid sequence of SEQ ID NOs:624, 625, and 626, respectively, and a VL comprising an LCDR1, an LCDR2, and an LCDR3 having an amino acid sequence of SEQ ID NOs:627, 628, and 629, respectively;
bbb. a VH comprising an HCDR1, an HCDR2, and an HCDR3 having an amino acid sequence of SEQ ID NOs:630, 631, and 632, respectively, and a VL comprising an LCDR1, an LCDR2, and an LCDR3 having an amino acid sequence of SEQ ID NOs:633, 634, and 635, respectively;
ccc. a VH comprising an HCDR1, an HCDR2, and an HCDR3 having an amino acid sequence of SEQ ID NOs:636, 637, and 638, respectively, and a VL comprising an LCDR1, an LCDR2, and an LCDR3 having an amino acid sequence of SEQ ID NOs:639, 640, and 641, respectively;
ddd. a VH comprising an HCDR1, an HCDR2, and an HCDR3 having an amino acid sequence of SEQ ID NOs:642, 643, and 644, respectively, and a VL comprising an LCDR1, an LCDR2, and an LCDR3 having an amino acid sequence of SEQ ID NOs:645, 646, and 647, respectively;
eee. a VH comprising an HCDR1, an HCDR2, and an HCDR3 having an amino acid sequence of SEQ ID NOs:648, 649, and 650, respectively, and a VL comprising an LCDR1, an LCDR2, and an LCDR3 having an amino acid sequence of SEQ ID NOs:651, 652, and 653, respectively;
fff. a VH comprising an HCDR1, an HCDR2, and an HCDR3 having an amino acid sequence of SEQ ID NOs:654, 655, and 656, respectively, and a VL comprising an LCDR1, an LCDR2, and an LCDR3 having an amino acid sequence of SEQ ID NOs:657, 658, and 659, respectively;
ggg. a VH comprising an HCDR1, an HCDR2, and an HCDR3 having an amino acid sequence of SEQ ID NOs:666, 667, and 668, respectively, and a VL comprising an LCDR1, an LCDR2, and an LCDR3 having an amino acid sequence of SEQ ID NOs:669, 670, and 671,
hhh. a VH comprising an HCDR1, an HCDR2, and an HCDR3 having an amino acid sequence of SEQ ID NOs:900, 901, and 902, respectively, and a VL comprising an LCDR1, an LCDR2, and an LCDR3 having an amino acid sequence of SEQ ID NOs:903, 904, and 905, respectively;
iii. a VH comprising an HCDR1, an HCDR2, and an HCDR3 having an amino acid sequence of SEQ ID NOs:906, 907, and 908, respectively, and a VL comprising an LCDR1, an LCDR2, and an LCDR3 having an amino acid sequence of SEQ ID NOs:909, 910, and 911, respectively;
jjj. a VH comprising an HCDR1, an HCDR2, and an HCDR3 having an amino acid sequence of SEQ ID NOs:912, 913, and 914, respectively, and a VL comprising an LCDR1, an LCDR2, and an LCDR3 having an amino acid sequence of SEQ ID NOs:915, 916, and 917, respectively;
kkk. a VH comprising an HCDR1, an HCDR2, and an HCDR3 having an amino acid sequence of SEQ ID NOs:918, 919, and 920, respectively, and a VL comprising an LCDR1, an LCDR2, and an LCDR3 having an amino acid sequence of SEQ ID NOs:921, 922, and 923, respectively;
lll. a VH comprising an HCDR1, an HCDR2, and an HCDR3 having an amino acid sequence of SEQ ID NOs:936, 937, and 938, respectively, and a VL comprising an LCDR1, an LCDR2, and an LCDR3 having an amino acid sequence of SEQ ID NOs:939, 940, and 941, respectively;
mmm. a VH comprising an HCDR1, an HCDR2, and an HCDR3 having an amino acid sequence of SEQ ID NOs:960, 961, and 962, respectively, and a VL comprising an LCDR1, an LCDR2, and an LCDR3 having an amino acid sequence of SEQ ID NOs:963, 964, and 965, respectively;
nnn. a VH comprising an HCDR1, an HCDR2, and an HCDR3 having an amino acid sequence of SEQ ID NOs:966, 967, and 968, respectively, and a VL comprising an LCDR1, an LCDR2, and an LCDR3 having an amino acid sequence of SEQ ID NOs:969, 970, and 971, respectively;
ooo. a VH comprising an HCDR1, an HCDR2, and an HCDR3 having an amino acid sequence of SEQ ID NOs:972, 973, and 974, respectively, and a VL comprising an LCDR1, an LCDR2, and an LCDR3 having an amino acid sequence of SEQ ID NOs:975, 976, and 977, respectively;
ppp. a VH comprising an HCDR1, an HCDR2, and an HCDR3 having an amino acid sequence of SEQ ID NOs:978, 979, and 980, respectively, and a VL comprising an LCDR1, an LCDR2, and an LCDR3 having an amino acid sequence of SEQ ID NOs:981, 982, and 983,
qqq. a VH comprising an HCDR1, an HCDR2, and an HCDR3 having an amino acid sequence of SEQ ID NOs:984, 985, and 986, respectively, and a VL comprising an LCDR1, an LCDR2, and an LCDR3 having an amino acid sequence of SEQ ID NOs:987, 988, and 989, respectively;
rrr. a VH comprising an HCDR1, an HCDR2, and an HCDR3 having an amino acid sequence of SEQ ID NOs:990, 991, and 992, respectively, and a VL comprising an LCDR1, an LCDR2, and an LCDR3 having an amino acid sequence of SEQ ID NOs:993, 994, and 995, respectively;
sss. a VH comprising an HCDR1, an HCDR2, and an HCDR3 having an amino acid sequence of SEQ ID NOs:996, 997, and 998, respectively, and a VL comprising an LCDR1, an LCDR2, and an LCDR3 having an amino acid sequence of SEQ ID NOs:999, 1000, and 1001, respectively;
ttt. a VH comprising an HCDR1, an HCDR2, and an HCDR3 having an amino acid sequence of SEQ ID NOs:1002, 1003, and 1004, respectively, and a VL comprising an LCDR1, an LCDR2, and an LCDR3 having an amino acid sequence of SEQ ID NOs:1005, 1006, and 1007, respectively;
uuu. a VH comprising an HCDR1, an HCDR2, and an HCDR3 having an amino acid sequence of SEQ ID NOs:1008, 1009, and 1010, respectively, and a VL comprising an LCDR1, an LCDR2, and an LCDR3 having an amino acid sequence of SEQ ID NOs:1011, 1012, and 1013, respectively;
vvv. a VH comprising an HCDR1, an HCDR2, and an HCDR3 having an amino acid sequence of SEQ ID NOs:1014, 1015, and 1016, respectively, and a VL comprising an LCDR1, an LCDR2, and an LCDR3 having an amino acid sequence of SEQ ID NOs:1017, 1018, and 1019, respectively;
www. a VH comprising an HCDR1, an HCDR2, and an HCDR3 having an amino acid sequence of SEQ ID NOs:1020, 1021, and 1022, respectively, and a VL comprising an LCDR1, an LCDR2, and an LCDR3 having an amino acid sequence of SEQ ID NOs:1023, 1024, and 1025, respectively;
xxx. a VH comprising an HCDR1, an HCDR2, and an HCDR3 having an amino acid sequence of SEQ ID NOs:1026, 1027, and 1028, respectively, and a VL comprising an LCDR1, an LCDR2, and an LCDR3 having an amino acid sequence of SEQ ID NOs:1029, 1030, and 1031, respectively;
yyy. a VH comprising an HCDR1, an HCDR2, and an HCDR3 having an amino acid sequence of SEQ ID NOs:1032, 1033, and 1034, respectively, and a VL comprising an LCDR1, an LCDR2, and an LCDR3 having an amino acid sequence of SEQ ID NOs:1035, 1036, and 1037,
zzz. a VH comprising an HCDR1, an HCDR2, and an HCDR3 having an amino acid sequence of SEQ ID NOs:1038, 1039, and 1040, respectively, and a VL comprising an LCDR1, an LCDR2, and an LCDR3 having an amino acid sequence of SEQ ID NOs:1041, 1042, and 1043, respectively;
aaaa. a VH comprising an HCDR1, an HCDR2, and an HCDR3 having an amino acid sequence of SEQ ID NOs: 1050, 1051, and 1052, respectively, and a VL comprising an LCDR1, an LCDR2, and an LCDR3 having an amino acid sequence of SEQ ID NOs:1053, 1054, and 1055, respectively;
bbbb. a VH comprising an HCDR1, an HCDR2, and an HCDR3 having an amino acid sequence of SEQ ID NOs:1056, 1057, and 1058, respectively, and a VL comprising an LCDR1, an LCDR2, and an LCDR3 having an amino acid sequence of SEQ ID NOs:1059, 1060, and 1061, respectively;
cccc. a VH comprising an HCDR1, an HCDR2, and an HCDR3 having an amino acid sequence of SEQ ID NOs:1062, 1063, and 1064, respectively, and a VL comprising an LCDR1, an LCDR2, and an LCDR3 having an amino acid sequence of SEQ ID NOs:1065, 1066, and 1067, respectively;
dddd. a VH comprising an HCDR1, an HCDR2, and an HCDR3 having an amino acid sequence of SEQ ID NOs:1068, 1069, and 1070, respectively, and a VL comprising an LCDR1, an LCDR2, and an LCDR3 having an amino acid sequence of SEQ ID NOs:1071, 1072, and 1073, respectively;
eeee. a VH comprising an HCDR1, an HCDR2, and an HCDR3 having an amino acid sequence of SEQ ID NOs: 1080, 1081, and 1082, respectively, and a VL comprising an LCDR1, an LCDR2, and an LCDR3 having an amino acid sequence of SEQ ID NOs:1083, 1084, and 1085, respectively;
ffff. a VH comprising an HCDR1, an HCDR2, and an HCDR3 having an amino acid sequence of SEQ ID NOs:1086, 1087, and 1088, respectively, and a VL comprising an LCDR1, an LCDR2, and an LCDR3 having an amino acid sequence of SEQ ID NOs:1089, 1090, and 1091, respectively;
gggg. a VH comprising an HCDR1, an HCDR2, and an HCDR3 having an amino acid sequence of SEQ ID NOs:1092, 1093, and 1094, respectively, and a VL comprising an LCDR1, an LCDR2, and an LCDR3 having an amino acid sequence of SEQ ID NOs:1095, 1096, and 1097, respectively;
hhhh. a VH comprising an HCDR1, an HCDR2, and an HCDR3 having an amino acid sequence of SEQ ID NOs:1098, 1099, and 1100, respectively, and a VL comprising an LCDR1, an LCDR2, and an LCDR3 having an amino acid sequence of SEQ ID NOs:1101, 1102, and 1103,
iiii. a VH comprising an HCDR1, an HCDR2, and an HCDR3 having an amino acid sequence of SEQ ID NOs:1104, 1105, and 1106, respectively, and a VL comprising an LCDR1, an LCDR2, and an LCDR3 having an amino acid sequence of SEQ ID NOs:1107, 1108, and 1109, respectively;
jjjj. a VH comprising an HCDR1, an HCDR2, and an HCDR3 having an amino acid sequence of SEQ ID NOs:1110, 1111, and 1112, respectively, and a VL comprising an LCDR1, an LCDR2, and an LCDR3 having an amino acid sequence of SEQ ID NOs:1113, 1114, and 1115, respectively;
kkkk. a VH comprising an HCDR1, an HCDR2, and an HCDR3 having an amino acid sequence of SEQ ID NOs:1116, 1117, and 1118, respectively, and a VL comprising an LCDR1, an LCDR2, and an LCDR3 having an amino acid sequence of SEQ ID NOs:1119, 1120, and 1121, respectively;
llll. a VH comprising an HCDR1, an HCDR2, and an HCDR3 having an amino acid sequence of SEQ ID NOs:1122, 1123, and 1124, respectively, and a VL comprising an LCDR1, an LCDR2, and an LCDR3 having an amino acid sequence of SEQ ID NOs:1125, 1126, and 1127, respectively;
mmmm. a VH comprising an HCDR1, an HCDR2, and an HCDR3 having an amino acid sequence of SEQ ID NOs:1128, 1129, and 1130, respectively, and a VL comprising an LCDR1, an LCDR2, and an LCDR3 having an amino acid sequence of SEQ ID NOs:1131, 1132, and 1133, respectively;
nnnn. a VH comprising an HCDR1, an HCDR2, and an HCDR3 having an amino acid sequence of SEQ ID NOs:1134, 1135, and 1136, respectively, and a VL comprising an LCDR1, an LCDR2, and an LCDR3 having an amino acid sequence of SEQ ID NOs:1137, 1138, and 1139, respectively;
oooo. a VH comprising an HCDR1, an HCDR2, and an HCDR3 having an amino acid sequence of SEQ ID NOs: 1140, 1141, and 1142, respectively, and a VL comprising an LCDR1, an LCDR2, and an LCDR3 having an amino acid sequence of SEQ ID NOs:1143, 1144, and 1145, respectively;
pppp. a VH comprising an HCDR1, an HCDR2, and an HCDR3 having an amino acid sequence of SEQ ID NOs:1146, 1147, and 1148, respectively, and a VL comprising an LCDR1, an LCDR2, and an LCDR3 having an amino acid sequence of SEQ ID NOs:1149, 1150, and 1151, respectively;
qqqq. a VH comprising an HCDR1, an HCDR2, and an HCDR3 having an amino acid sequence of SEQ ID NOs:1152, 1153, and 1154, respectively, and a VL comprising an LCDR1, an LCDR2, and an LCDR3 having an amino acid sequence of SEQ ID NOs:1155, 1156, and 1157,
rrrr. a VH comprising an HCDR1, an HCDR2, and an HCDR3 having an amino acid sequence of SEQ ID NOs:1158, 1159, and 1160, respectively, and a VL comprising an LCDR1, an LCDR2, and an LCDR3 having an amino acid sequence of SEQ ID NOs:1161, 1162, and 1163, respectively;
ssss. a VH comprising an HCDR1, an HCDR2, and an HCDR3 having an amino acid sequence of SEQ ID NOs:1164, 1165, and 1166, respectively, and a VL comprising an LCDR1, an LCDR2, and an LCDR3 having an amino acid sequence of SEQ ID NOs:1167, 1168, and 1169, respectively;
tttt. a VH comprising an HCDR1, an HCDR2, and an HCDR3 having an amino acid sequence of SEQ ID NOs:1170, 1171, and 1172, respectively, and a VL comprising an LCDR1, an LCDR2, and an LCDR3 having an amino acid sequence of SEQ ID NOs:1173, 1174, and 1175, respectively;
uuuu. a VH comprising an HCDR1, an HCDR2, and an HCDR3 having an amino acid sequence of SEQ ID NOs:1176, 1177, and 1178, respectively, and a VL comprising an LCDR1, an LCDR2, and an LCDR3 having an amino acid sequence of SEQ ID NOs:1179, 1180, and 1181, respectively;
vvvv. a VH comprising an HCDR1, an HCDR2, and an HCDR3 having an amino acid sequence of SEQ ID NOs:1182, 1183, and 1184, respectively, and a VL comprising an LCDR1, an LCDR2, and an LCDR3 having an amino acid sequence of SEQ ID NOs:1185, 1186, and 1187, respectively;
wwww. a VH comprising an HCDR1, an HCDR2, and an HCDR3 having an amino acid sequence of SEQ ID NOs:1188, 1189, and 1190, respectively, and a VL comprising an LCDR1, an LCDR2, and an LCDR3 having an amino acid sequence of SEQ ID NOs:1191, 1192, and 1193, respectively;
xxxx. a VH comprising an HCDR1, an HCDR2, and an HCDR3 having an amino acid sequence of SEQ ID NOs:1194, 1195, and 1196, respectively, and a VL comprising an LCDR1, an LCDR2, and an LCDR3 having an amino acid sequence of SEQ ID NOs:1197, 1198, and 1199, respectively;
yyyy. a VH comprising an HCDR1, an HCDR2, and an HCDR3 having an amino acid sequence of SEQ ID NOs:1200, 1201, and 1202, respectively, and a VL comprising an LCDR1, an LCDR2, and an LCDR3 having an amino acid sequence of SEQ ID NOs:1203, 1204, and 1205, respectively;
zzzz. a VH comprising an HCDR1, an HCDR2, and an HCDR3 having an amino acid sequence of SEQ ID NOs:1206, 1207, and 1208, respectively, and a VL comprising an LCDR1, an LCDR2, and an LCDR3 having an amino acid sequence of SEQ ID NOs:1209, 1210, and 1211, respectively; or
aaaaa. a VH comprising an HCDR1, an HCDR2, and an HCDR3 having an amino acid sequence of SEQ ID NOs:1212, 1213, and 1214, respectively, and a VL comprising an LCDR1, an LCDR2, and an LCDR3 having an amino acid sequence of SEQ ID NOs:1215, 1216, and 1217, respectively.

27. The isolated monoclonal antibody or antigen binding fragment thereof of any of aspects 25 or 26 comprising:
a. a VH comprising the amino acid sequence of SEQ ID NO:138, and a VL comprising the amino acid sequence of SEQ ID NO:139;
b. a VH comprising the amino acid sequence of SEQ ID NO:142, and a VL comprising the amino acid sequence of SEQ ID NO:143;
c. a VH comprising the amino acid sequence of SEQ ID NO:144, and a VL comprising the amino acid sequence of SEQ ID NO:145;
d. a VH comprising the amino acid sequence of SEQ ID NO:148, and a VL comprising the amino acid sequence of SEQ ID NO:149;
e. a VH comprising the amino acid sequence of SEQ ID NO:150, and a VL comprising the amino acid sequence of SEQ ID NO:151;
f. a VH comprising the amino acid sequence of SEQ ID NO:152, and a VL comprising the amino acid sequence of SEQ ID NO:153;
g. a VH comprising the amino acid sequence of SEQ ID NO:154, and a VL comprising the amino acid sequence of SEQ ID NO:155;
h. a VH comprising the amino acid sequence of SEQ ID NO:156, and a VL comprising the amino acid sequence of SEQ ID NO:157;
i. a VH comprising the amino acid sequence of SEQ ID NO:158, and a VL comprising the amino acid sequence of SEQ ID NO:159;
j. a VH comprising the amino acid sequence of SEQ ID NO:160, and a VL comprising the amino acid sequence of SEQ ID NO:161;
k. a VH comprising the amino acid sequence of SEQ ID NO:162, and a VL comprising the amino acid sequence of SEQ ID NO:163;
l. a VH comprising the amino acid sequence of SEQ ID NO:164, and a VL comprising the amino acid sequence of SEQ ID NO:165;
m. a VH comprising the amino acid sequence of SEQ ID NO:166, and a VL comprising the amino acid sequence of SEQ ID NO:167;
n. a VH comprising the amino acid sequence of SEQ ID NO:168, and a VL comprising the amino acid sequence of SEQ ID NO:169;
o. a VH comprising the amino acid sequence of SEQ ID NO:170, and a VL comprising the amino acid sequence of SEQ ID NO:171;
p. a VH comprising the amino acid sequence of SEQ ID NO:172, and a VL comprising the amino acid sequence of SEQ ID NO:173;
q. a VH comprising the amino acid sequence of SEQ ID NO:174, and a VL comprising the amino acid sequence of SEQ ID NO:175;
r. a VH comprising the amino acid sequence of SEQ ID NO:176, and a VL comprising the amino acid sequence of SEQ ID NO:177;
s. a VH comprising the amino acid sequence of SEQ ID NO:178, and a VL comprising the amino acid sequence of SEQ ID NO:179;
t. a VH comprising the amino acid sequence of SEQ ID NO:180, and a VL comprising the amino acid sequence of SEQ ID NO:181;
u. a VH comprising the amino acid sequence of SEQ ID NO:182, and a VL comprising the amino acid sequence of SEQ ID NO: 183;
v. a VH comprising the amino acid sequence of SEQ ID NO:186, and a VL comprising the amino acid sequence of SEQ ID NO:187;
w. a VH comprising the amino acid sequence of SEQ ID NO:188, and a VL comprising the amino acid sequence of SEQ ID NO:189;
x. a VH comprising the amino acid sequence of SEQ ID NO:190, and a VL comprising the amino acid sequence of SEQ ID NO:191;
y. a VH comprising the amino acid sequence of SEQ ID NO:192, and a VL comprising the amino acid sequence of SEQ ID NO:193;
z. a VH comprising the amino acid sequence of SEQ ID NO:194, and a VL comprising the amino acid sequence of SEQ ID NO:195;
aa. a VH comprising the amino acid sequence of SEQ ID NO:196, and a VL comprising the amino acid sequence of SEQ ID NO:197;
bb. a VH comprising the amino acid sequence of SEQ ID NO:198, and a VL comprising the amino acid sequence of SEQ ID NO:199;
cc. a VH comprising the amino acid sequence of SEQ ID NO:200, and a VL comprising the amino acid sequence of SEQ ID NO:201;
dd. a VH comprising the amino acid sequence of SEQ ID NO:202, and a VL comprising the amino acid sequence of SEQ ID NO:203;
ee. a VH comprising the amino acid sequence of SEQ ID NO:204, and a VL comprising the amino acid sequence of SEQ ID NO:205;
ff. a VH comprising the amino acid sequence of SEQ ID NO:206, and a VL comprising the amino acid sequence of SEQ ID NO:207;
gg. a VH comprising the amino acid sequence of SEQ ID NO:208, and a VL comprising the amino acid sequence of SEQ ID NO:209;
hh. a VH comprising the amino acid sequence of SEQ ID NO:210, and a VL comprising the amino acid sequence of SEQ ID NO:211;
ii. a VH comprising the amino acid sequence of SEQ ID NO:212, and a VL comprising the amino acid sequence of SEQ ID NO:213;
jj. a VH comprising the amino acid sequence of SEQ ID NO:214, and a VL comprising the amino acid sequence of SEQ ID NO:215;
kk. a VH comprising the amino acid sequence of SEQ ID NO:216, and a VL comprising the amino acid sequence of SEQ ID NO:217;
ll. a VH comprising the amino acid sequence of SEQ ID NO:218, and a VL comprising the amino acid sequence of SEQ ID NO:219;
mm. a VH comprising the amino acid sequence of SEQ ID NO:220, and a VL comprising the amino acid sequence of SEQ ID NO:221;
nn. a VH comprising the amino acid sequence of SEQ ID NO:222, and a VL comprising the amino acid sequence of SEQ ID NO:223;
oo. a VH comprising the amino acid sequence of SEQ ID NO:226, and a VL comprising the amino acid sequence of SEQ ID NO:227;
pp. a VH comprising the amino acid sequence of SEQ ID NO:228, and a VL comprising the amino acid sequence of SEQ ID NO:229;
qq. a VH comprising the amino acid sequence of SEQ ID NO:230, and a VL comprising the amino acid sequence of SEQ ID NO:231;
rr. a VH comprising the amino acid sequence of SEQ ID NO:232, and a VL comprising the amino acid sequence of SEQ ID NO:233;
ss. a VH comprising the amino acid sequence of SEQ ID NO:234, and a VL comprising the amino acid sequence of SEQ ID NO:235;
tt. a VH comprising the amino acid sequence of SEQ ID NO:236, and a VL comprising the amino acid sequence of SEQ ID NO:237;
uu. a VH comprising the amino acid sequence of SEQ ID NO:238, and a VL comprising the amino acid sequence of SEQ ID NO:239;
vv. a VH comprising the amino acid sequence of SEQ ID NO:240, and a VL comprising the amino acid sequence of SEQ ID NO:241;
ww. a VH comprising the amino acid sequence of SEQ ID NO:246, and a VL comprising the amino acid sequence of SEQ ID NO:247;
xx. a VH comprising the amino acid sequence of SEQ ID NO:248, and a VL comprising the amino acid sequence of SEQ ID NO:249;
yy. a VH comprising the amino acid sequence of SEQ ID NO:250, and a VL comprising the amino acid sequence of SEQ ID NO:251;
zz. a VH comprising the amino acid sequence of SEQ ID NO:252, and a VL comprising the amino acid sequence of SEQ ID NO:253;
aaa. a VH comprising the amino acid sequence of SEQ ID NO:254, and a VL comprising the amino acid sequence of SEQ ID NO:255;
bbb. a VH comprising the amino acid sequence of SEQ ID NO:256, and a VL comprising the amino acid sequence of SEQ ID NO:257;
ccc. a VH comprising the amino acid sequence of SEQ ID NO:258, and a VL comprising the amino acid sequence of SEQ ID NO:259;
ddd. a VH comprising the amino acid sequence of SEQ ID NO:260, and a VL comprising the amino acid sequence of SEQ ID NO:261;
eee. a VH comprising the amino acid sequence of SEQ ID NO:262, and a VL comprising the amino acid sequence of SEQ ID NO:263;
fff. a VH comprising the amino acid sequence of SEQ ID NO:264, and a VL comprising the amino acid sequence of SEQ ID NO:265;
ggg. a VH comprising the amino acid sequence of SEQ ID NO:268, and a VL comprising the amino acid sequence of SEQ ID NO:269;
hhh. a VH comprising the amino acid sequence of SEQ ID NO:786, and a VL comprising the amino acid sequence of SEQ ID NO:787;
iii. a VH comprising the amino acid sequence of SEQ ID NO:788, and a VL comprising the amino acid sequence of SEQ ID NO:789;
jjj. a VH comprising the amino acid sequence of SEQ ID NO:790, and a VL comprising the amino acid sequence of SEQ ID NO:791;
kkk. a VH comprising the amino acid sequence of SEQ ID NO:792, and a VL comprising the amino acid sequence of SEQ ID NO:793;
lll. a VH comprising the amino acid sequence of SEQ ID NO:798, and a VL comprising the amino acid sequence of SEQ ID NO:799;
mmm. a VH comprising the amino acid sequence of SEQ ID NO:806, and a VL comprising the amino acid sequence of SEQ ID NO:807;
nnn. a VH comprising the amino acid sequence of SEQ ID NO:808, and a VL comprising the amino acid sequence of SEQ ID NO:809;
ooo. a VH comprising the amino acid sequence of SEQ ID NO:810, and a VL comprising the amino acid sequence of SEQ ID NO:811;
ppp. a VH comprising the amino acid sequence of SEQ ID NO:812, and a VL comprising the amino acid sequence of SEQ ID NO:813;
qqq. a VH comprising the amino acid sequence of SEQ ID NO:814, and a VL comprising the amino acid sequence of SEQ ID NO:815;
rrr. a VH comprising the amino acid sequence of SEQ ID NO:816, and a VL comprising the amino acid sequence of SEQ ID NO:817;
sss. a VH comprising the amino acid sequence of SEQ ID NO:818, and a VL comprising the amino acid sequence of SEQ ID NO:819;
ttt. a VH comprising the amino acid sequence of SEQ ID NO:820, and a VL comprising the amino acid sequence of SEQ ID NO:821;
uuu. a VH comprising the amino acid sequence of SEQ ID NO:822, and a VL comprising the amino acid sequence of SEQ ID NO:823;
vvv. a VH comprising the amino acid sequence of SEQ ID NO:824, and a VL comprising the amino acid sequence of SEQ ID NO:825;
www.a VH comprising the amino acid sequence of SEQ ID NO:826, and a VL comprising the amino acid sequence of SEQ ID NO:827;
xxx. a VH comprising the amino acid sequence of SEQ ID NO:828, and a VL comprising the amino acid sequence of SEQ ID NO:829;
yyy. a VH comprising the amino acid sequence of SEQ ID NO:830, and a VL comprising the amino acid sequence of SEQ ID NO:831;
zzz. a VH comprising the amino acid sequence of SEQ ID NO:832, and a VL comprising the amino acid sequence of SEQ ID NO:833;
aaaa. a VH comprising the amino acid sequence of SEQ ID NO:836, and a VL comprising the amino acid sequence of SEQ ID NO:837;
bbbb. a VH comprising the amino acid sequence of SEQ ID NO:838, and a VL comprising the amino acid sequence of SEQ ID NO:839;
cccc. a VH comprising the amino acid sequence of SEQ ID NO:840, and a VL comprising the amino acid sequence of SEQ ID NO:841;
dddd. a VH comprising the amino acid sequence of SEQ ID NO: 842, and a VL comprising the amino acid sequence of SEQ ID NO:843;
eeee. a VH comprising the amino acid sequence of SEQ ID NO:846, and a VL comprising the amino acid sequence of SEQ ID NO:847;
ffff. a VH comprising the amino acid sequence of SEQ ID NO:848, and a VL comprising the amino acid sequence of SEQ ID NO:849;
gggg. a VH comprising the amino acid sequence of SEQ ID NO:850, and a VL comprising the amino acid sequence of SEQ ID NO:851;
hhhh. a VH comprising the amino acid sequence of SEQ ID NO:852, and a VL comprising the amino acid sequence of SEQ ID NO:853;
iiii. a VH comprising the amino acid sequence of SEQ ID NO:854, and a VL comprising the amino acid sequence of SEQ ID NO:855;
jjjj. a VH comprising the amino acid sequence of SEQ ID NO:856, and a VL comprising the amino acid sequence of SEQ ID NO:857;
kkkk. a VH comprising the amino acid sequence of SEQ ID NO:858, and a VL comprising the amino acid sequence of SEQ ID NO:859;
llll. a VH comprising the amino acid sequence of SEQ ID NO:860, and a VL comprising the amino acid sequence of SEQ ID NO:861;
mmmm. a VH comprising the amino acid sequence of SEQ ID NO:862, and a VL comprising the amino acid sequence of SEQ ID NO:863;
nnnn. a VH comprising the amino acid sequence of SEQ ID NO:864, and a VL comprising the amino acid sequence of SEQ ID NO:865;
oooo. a VH comprising the amino acid sequence of SEQ ID NO:866, and a VL comprising the amino acid sequence of SEQ ID NO:867;
pppp. a VH comprising the amino acid sequence of SEQ ID NO:868, and a VL comprising the amino acid sequence of SEQ ID NO:869;
qqqq. a VH comprising the amino acid sequence of SEQ ID NO:870, and a VL comprising the amino acid sequence of SEQ ID NO:871;
rrrr. a VH comprising the amino acid sequence of SEQ ID NO:872, and a VL comprising the amino acid sequence of SEQ ID NO:873;
ssss. a VH comprising the amino acid sequence of SEQ ID NO:874, and a VL comprising the amino acid sequence of SEQ ID NO:875;
tttt. a VH comprising the amino acid sequence of SEQ ID NO:876, and a VL comprising the amino acid sequence of SEQ ID NO:877;
uuuu. a VH comprising the amino acid sequence of SEQ ID NO:878, and a VL comprising the amino acid sequence of SEQ ID NO:879;
vvvv. a VH comprising the amino acid sequence of SEQ ID NO:880, and a VL comprising the amino acid sequence of SEQ ID NO:881;
wwww. a VH comprising the amino acid sequence of SEQ ID NO:882, and a VL comprising the amino acid sequence of SEQ ID NO:883;
xxxx. a VH comprising the amino acid sequence of SEQ ID NO:884, and a VL comprising the amino acid sequence of SEQ ID NO:885;
yyyy. a VH comprising the amino acid sequence of SEQ ID NO:886, and a VL comprising the amino acid sequence of SEQ ID NO:887;
zzzz. a VH comprising the amino acid sequence of SEQ ID NO:888, and a VL comprising the amino acid sequence of SEQ ID NO:889; or aaaaa. a VH comprising the amino acid sequence of SEQ ID NO:890, and a VL comprising the amino acid sequence of SEQ ID NO:891.

28. The antibody or fragment of any of aspects 1-27, wherein the antibody is an IgG.

29. The antibody or fragment of any of aspects 1-28, wherein the antibody is a humanized or chimeric antibody or fragment thereof.

30. The isolated antibody or fragment of any of aspects 1-28 which is an antibody or fragment thereof wherein said isolated antibody is the form of an antibody F(ab′)2, scFv fragment, domain antibody, minibody, diabody, triabody or tetrabody.

31. The antibody of any of aspects 1-30, wherein the antibody is genetically fused or chemically conjugated to an agent,
wherein optionally the agent a detectable substance or is a drug,
wherein optionally the detectable substance is selected from enzymes, prosthetic groups, fluorescent materials, luminescent materials, bioluminescent materials or chemiluminescent materials,
wherein optionally the enzymes are selected from horseradish peroxidase, alkaline phosphatase, beta-galactosidase, or acetylcholinesterase;
wherein optionally the prosthetic groups are selected from streptavidin/biotin or avidin/biotin;
wherein optionally the fluorescent materials are selected from umbelliferone, fluorescein, fluorescein isothiocynate, rhodamine, dichlorotriazinylamine fluorescein, dansyl chloride, or phycoerythrin;
wherein optionally the luminescent material is luminol;
wherein optionally the bioluminescent materials are selected from luciferase, luciferin, or aequorin; and
wherein optionally the chemiluminescent materials are selected from 225 Acγ-emitting, Auger-emitting, β-emitting, an alpha-emitting or positron-emitting radioactive isotope.

32. A nucleic acid molecule encoding the antibody of any of aspects 1-30 or a variable region or antigen binding fragment thereof.

33. A vector comprising the nucleic acid molecule of aspect 32.

34. A host cell transformed with the vector of aspect 33.

35. A composition comprising the antibody of any of aspects 1-31, the nucleic acid molecule of claim 30, or the vector of claim 31, and a pharmaceutically acceptable excipient.

36. A kit comprising the antibody of any of aspects 1-31.

37. A method for detecting, selecting and/or enriching interleukin-23 receptor (IL-23R) in a sample comprising contacting the sample with the antibody of any of aspects 1 to 31,
wherein optionally the method comprising using the antibody of any of aspects 1 to 31 in an immunohistochemistry (IHC) assay, an immunocytochemistry (ICC) assay, an immunoblotting assay, an immunoprecipitation assay, a flow cytometry assay, an ELISA, a radioimmunoassay, a mass spectrometry assay, or high throughput screening assay;
wherein:
optionally the flow cytometry assay comprises three-steps of staining: (1) first, incubating the sample with the antibody of any of aspects 1 to 31; (2) second, incubating the sample with biotinylated anti-mouse IgG₂ₐ; (3) third, incubating the sample with streptavidin-PE; and wherein optionally, to establish the background for the flow cytometry assay, cells incubated with buffer alone or the negative control antibody are used as a control group in step (1), and cells incubated without the biotinylated anti-mouse IgG₂ₐ secondary reagent are used as an additional control group in step (2).

38. A method for detecting, selecting and/or enriching interleukin-23 receptor (IL-23R) in a sample comprising contacting the sample with an antibody that binds to the same epitope as the antibody of any of aspects 1 to 31, or an antibody that binds IL-23R competitively with the antibody of any of aspects 1 to 31.

39. The method of aspect 38, comprising contacting the sample with an antibody that binds to the same epitope and/or binds IL-23R competitively with the antibody comprising:
a. a VH comprising the amino acid sequence of SEQ ID NO:202 and a VL comprising the amino acid sequence of SEQ ID NO:203;
b. a VH comprising the amino acid sequence of SEQ ID NO:252 and a VL comprising the amino acid sequence of SEQ ID NO:253; or
c. a VH comprising the amino acid sequence of SEQ ID NO:156 and a VL comprising the amino acid sequence of SEQ ID NO:157.

40. The method of aspect 38, comprising contacting the sample with an antibody that binds to the same epitope and/or binds IL-23R competitively with the antibody comprising:
a. a VH comprising the amino acid sequence of SEQ ID NO:192 and a VL comprising the amino acid sequence of SEQ ID NO:193;
b. a VH comprising the amino acid sequence of SEQ ID NO:194 and a VL comprising the amino acid sequence of SEQ ID NO:195;
c. a VH comprising the amino acid sequence of SEQ ID NO:200 and a VL comprising the amino acid sequence of SEQ ID NO:201;
d. a VH comprising the amino acid sequence of SEQ ID NO:218 and a VL comprising the amino acid sequence of SEQ ID NO:219;
e. a VH comprising the amino acid sequence of SEQ ID NO:222 and a VL comprising the amino acid sequence of SEQ ID NO:223;
f. a VH comprising the amino acid sequence of SEQ ID NO:260 and a VL comprising the amino acid sequence of SEQ ID NO:261;
g. a VH comprising the amino acid sequence of SEQ ID NO:264 and a VL comprising the amino acid sequence of SEQ ID NO:265;
h. a VH comprising the amino acid sequence of SEQ ID NO:154 and a VL comprising the amino acid sequence of SEQ ID NO:155;
i. a VH comprising the amino acid sequence of SEQ ID NO:160 and a VL comprising the amino acid sequence of SEQ ID NO:161; or
j. a VH comprising the amino acid sequence of SEQ ID NO:164 and a VL comprising the amino acid sequence of SEQ ID NO:165.

41. The method of aspect 38, comprising contacting the sample with an antibody that binds to the same epitope and/or binds IL-23R competitively with the antibody comprising:
a. a VH comprising the amino acid sequence of SEQ ID NO:786 and a VL comprising the amino acid sequence of SEQ ID NO:787;
b. a VH comprising the amino acid sequence of SEQ ID NO:790 and a VL comprising the amino acid sequence of SEQ ID NO:791;
c. a VH comprising the amino acid sequence of SEQ ID NO:792 and a VL comprising the amino acid sequence of SEQ ID NO:793;
d. a VH comprising the amino acid sequence of SEQ ID NO:236 and a VL comprising the amino acid sequence of SEQ ID NO:237;
e. a VH comprising the amino acid sequence of SEQ ID NO:826 and a VL comprising the amino acid sequence of SEQ ID NO:827; or
f. a VH comprising the amino acid sequence of SEQ ID NO:828 and a VL comprising the amino acid sequence of SEQ ID NO:829.

42. The method of aspect 38, comprising contacting the sample with an antibody that binds to the same epitope and/or binds IL-23R competitively with the antibody comprising:
a. a VH comprising the amino acid sequence of SEQ ID NO:196 and a VL comprising the amino acid sequence of SEQ ID NO:197;
b. a VH comprising the amino acid sequence of SEQ ID NO:262 and a VL comprising the amino acid sequence of SEQ ID NO:263; or
c. a VH comprising the amino acid sequence of SEQ ID NO:152 and a VL comprising the amino acid sequence of SEQ ID NO:153.

43. The method of aspect 38, comprising contacting the sample with an antibody that binds to the same epitope and/or binds IL-23R competitively with the antibody comprising:
a. a VH comprising the amino acid sequence of SEQ ID NO:798 and a VL comprising the amino acid sequence of SEQ ID NO:799.

44. The method of aspect 38, comprising contacting the sample with an antibody that binds to the same epitope and/or binds IL-23R competitively with the antibody comprising:
a. a VH comprising the amino acid sequence of SEQ ID NO:206 and a VL comprising the amino acid sequence of SEQ ID NO:207;
b. a VH comprising the amino acid sequence of SEQ ID NO:250 and a VL comprising the amino acid sequence of SEQ ID NO:251;
c. a VH comprising the amino acid sequence of SEQ ID NO:144 and a VL comprising the amino acid sequence of SEQ ID NO:145; or
d. a VH comprising the amino acid sequence of SEQ ID NO:170 and a VL comprising the amino acid sequence of SEQ ID NO:171.

45. The method of aspect 38, comprising contacting the sample with an antibody that binds to the same epitope and/or binds IL-23R competitively with the antibody comprising:
a. a VH comprising the amino acid sequence of SEQ ID NO:150 and a VL comprising the amino acid sequence of SEQ ID NO:151.

46. The method of aspect 38, comprising contacting the sample with an antibody that binds to the same epitope and/or binds IL-23R competitively with the antibody comprising:
a. a VH comprising the amino acid sequence of SEQ ID NO:198 and a VL comprising the amino acid sequence of SEQ ID NO:199; or
b. a VH comprising the amino acid sequence of SEQ ID NO:240 and a VL comprising the amino acid sequence of SEQ ID NO:241.

47. The method of aspect 38, comprising contacting the sample with an antibody that binds to the same epitope and/or binds IL-23R competitively with the antibody comprising:
a. a VH comprising the amino acid sequence of SEQ ID NO:204 and a VL comprising the amino acid sequence of SEQ ID NO:205; or
b. a VH comprising the amino acid sequence of SEQ ID NO:884 and a VL comprising the amino acid sequence of SEQ ID NO:885.

48. The method of any of aspects 37 to 47, wherein the method is for detecting, selecting and/or enriching human IL-23R.

49. The method of any of aspects 37 to 47, wherein the method is for detecting, selecting and/or enriching cells that express IL-23R.

50. The method of any of aspects 37 to 47, wherein the method is for detecting denatured IL-23R.

51. The method of any of aspects 37 to 47, wherein the method comprising contacting the sample with two or more antibodies each being the antibody of any of aspects1 to 29.

52. The method of aspect 49, wherein the two or more antibodies bind to different epitopes of IL-23R.

53. The method of any of aspects 37 to 52, wherein the sample is from a human subject.

54. The method of aspect 53, wherein the human subject has a disease or disorder.

55. The method of aspect 54, wherein the disease or disorder is an IL-23R associated disease or disorder.

56. The method of aspect 53, wherein the human subject is a healthy human subject.

57. A method of antagonizing or inhibiting IL-23R in a cell comprising contacting the cell with the antibody of any of aspects 1 to 31.

58. A method of treating a disease or disorder in a subject, comprising administering to the subject the composition of aspect 35 or the antibody of any of aspects 1 to 31.

59. The method of aspect 58, wherein the disease or disorder is an IL-23R associated disease or disorder.

60. The method according to aspect 58, wherein the disease or disorder is associated with inflammatory, autoimmune inflammation diseases and/or related disorders.

61. The method according to aspect 60, wherein the disease or disorder associated with inflammatory, autoimmune inflammation diseases and/or related disorders is selected from multiple sclerosis, asthma, rheumatoid arthritis, inflammation of the gut, inflammatory bowel diseases (IBDs), juvenile IBD, adolescent IBD, Crohn's disease, ulcerative colitis, Celiac disease (nontropical Sprue), microscopic colitis, collagenous colitis, eosinophilic gastroenteritis/esophagitis, colitis associated with radio- or chemo-therapy, colitis associated with disorders of innate immunity as in leukocyte adhesion deficiency-1, sarcoidosis, Systemic Lupus Erythematosus, ankylosing spondylitis (axial spondyloarthritis), psoriatic arthritis, psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, enteropathy associated with seronegative arthropathies, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

62. The method of aspect 61, wherein the disease or disorder is associated with an autoimmune disease is selected from Ulcerative colitis (UC), Crohn's Disease (CD), psoriasis (PsO), or psoriatic arthritis (PsA).

63. Use of an antibody or antigen binding fragment thereof of any of aspects 1-31 or a composition of aspect 35 in the manufacture of a medicament for treatment of an IL-23R associated disease or disorder, particularly a disease or disorder associated with inflammatory, autoimmune inflammation diseases and/or related disorders.

64. The antibody or antigen binding fragment of any of aspects 1-31 or the composition of aspect 35 for use in the treatment of an IL-23R associated disease or disorder, particularly a disease or disorder associated with inflammatory, autoimmune inflammation diseases and/or related disorders

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### IL-23R Antibody Discovery and Characterization Summary

Discovery of monoclonal antibodies (mAbs) that bind human interleukin (IL)-23 (IL-23) receptor was done by both wild type mouse immunization and phage display and selection using proprietary Janssen de novo human and mouse Fab libraries. Recombinant human IL-23 receptor (IL-23R) extracellular domain (ECD) protein (sequence shown in Table 3) was used as the antigen in both the immunization and phage display approaches, with the IL-23R ECD protein validated for IL-23 binding. A panel of human IL-23R binding mAbs was identified through both approaches that included both mouse antibodies from immunization and phage display and human antibodies from phage display only.

TABLE 3
Amino acid sequence of human IL-23R ECD

| | Sequence |
|---|---|
| Q5VWK51 | GITNINCSGHIWVEPATIFKMGMNISIYCQAAIKN |
| 24-355 | CQPRKLHFYKNGIKERFQITRINKTTARLWYKNFL |
| ECD | EPHASMYCTAECPKHFQETLICGKDISSGYPPDIP |
| | DEVTCVIYEYSGNMTCTWNAGKLTYIDTKYVVHVK |
| | SLETEEEQQYLTSSYINISTDSLQGGKKYLVWVQA |
| | ANALGMEESKQLQIHLDDIVIPSAAVISRAETINA |
| | TVPKTIIYWDSQTTIEKVSCEMRYKATTNQTWNVK |
| | EFDTNFTYVQQSEFYLEPNIKYVFQVRCQETGKRY |
| | WQPWSSLFFHKTPETVPQVTSKAFQHDTWNSGLTV |
| | ASISTGHLTSDNRGDIG |
| | (SEQ ID NO: 1) |

All human IL-23R binding mAbs discovered were cloned with mouse IgG₂ₐ and mouse kappa constant domains and expressed recombinantly from mammalian cells for detailed characterization. Those mAbs originally identified as mouse antibodies were full mouse mAbs when recombinantly produced and those originally identified as human antibodies were human/mouse chimeric mAbs when recombinantly produced. The recombinant IL-23R binding mAbs expressed in mammalian cells were characterized as unpurified proteins in crude mammalian expression supernatants, as purified proteins or in both forms, with much of the characterization not requiring purified protein. Characterization of the IL-23R mAbs included determination of affinity to human and rat IL-23R ECD protein, determination of binding to recombinant cells lines expressing cell surface human IL-23R or primary human cells expressing IL-23R and determination of the number of human IL-23R ECD epitope/competition bins covered by the mAbs in this panel.

The human IL-23R binding mAbs discovered, cloned, expressed, characterized and described here have a range of affinities to human IL-23R ECD. The antibodies demonstrate activities and binding capabilities which are unique to and/or better than other known IL-23R antibodies, including commercially available IL-23R antibodies. Most of the human IL-23R binding antibodies herein demonstrate and have higher affinity, some with substantially higher affinity, than the commercially available IL-23R mAbs tested. The antibodiesrecognize multiple distinct epitopes on the IL-23R extracellular domain. Some of the antibodies have cross-reactive binding to rat IL-23R ECD and bind to both human IL-23R and rat IL-23R, particularly binding to human IL-23R ECD and rat IL-23R ECD. Many of these mAbs show binding to both recombinant cell lines and primary human cells expressing human IL-23R, and demonstrate and have cell binding that is better than that of the commercially available IL-23R mAbs tested. The novel IL-23R antibodies provided herein comprise unique sequences and distinct and relevant binding characteristics and affinity, even among and between one another, as well as in comparison to other known and available antibodies including commercially available antibodies.

### Example 1: IL-23R mAb Discovery

Discovery of monoclonal antibodies (mAbs) that bind human interleukin (IL)-23 (IL-23) receptor was done by both wild type mouse immunization and phage display.

### Immunization Based IL-23R mAb Discovery

Recombinant human IL-23R extracellular (ECD) protein antigen was prepared with adjuvant for immunization. AJ and C3H mice were injected with prepared IL-23R antigen+CL413 IP on days 0, 14, 28, and 47. The surviving mice were euthanized on day 54, 7 days post final boost, and spleens and serum were harvested. Splenocytes from harvested spleens of 9 total individual mice, three A/J and six C3H, were stained with biotinylated human IL-23R/Fc and AF647 labeled ILT4/Fc (to exclude Fc binders). With gating set for IL-23R+ and ILT4/Fc−, a total of 7040 single B cells were sorted into 20-384 well plates containing single B cell (SBC) media. Six days post sort, supernatants were collected and B cells were lysed for all 20 plates of cultured single B cells. Supernatants were diluted 1:2 in PBS/BSA (0.4%) and stored at 4° C. Lysate plates were stored at −80° C. Supernatants were subsequently screened for binding to human IL-23R ECD protein and for IgG concentration using Meso Scale Discovery (MSD)-based assays.

876 SBC clones were identified with >10-fold IL-23R binding over background and the IgG quantification assay identified 451 clones with >2 ng/ml IgG, with 421 clones meeting both criteria. From these 421 clones, 384 were selected based on IL-23R binding over background being >63-fold and IgG expression being >10 ng/ml. The antibody heavy chain (HC) and light chain (LC) variable regions (VH and VL, respectively) were cloned from the frozen lysates of the cultured single B cells using a 5′ RACE V-region cloning method. VH and VL regions obtained were cloned with mouse IgG₂ₐ and mouse kappa constant regions, respectively, into separate mammalian cell expression vectors. Not all V-regions were successfully amplified and cloned. Plasmid DNA was prepared for all expression constructs generated for use in various scales of expression of the recombinant antibodies. Expression constructs were not sequenced and were considered non-clonal at this stage.

### Phage Display Based IL-23R mAb Discovery

Human IL-23 receptor binding Fabs were selected from in-house generated mouse de novo Fab-pIX phage display libraries or human de novo Fab-pIX display libraries as described in Shi et al., J Mol Biol 397:385-96, 2010 and Int. Pat. Publ. No. WO2009/085462; U.S. Pat. Publ. No. US2010/0021477, following standard protocols (Cheadle, E. J. et al. Antibody Engineering. 907, 645-666 (2012).

Phage panning. In the phage selections, biotinylated purified recombinant biotinylated human IL-23 receptor (IL-23R) extracellular domain (ECD) was used as the antigen to capture and immobilize the phage binders. 42 panning experiments (I23RXP1 to 36, I23RXP53 to 57) using the mouse libraries and 8 panning experiments (I23RXP37 and 38, I23RXP47 to 52) using the human libraries were run. In each panning experiment, a specific combination of library(s) and panning conditions was used. Briefly, the phage libraries and paramagnetic streptavidin (SA) beads were blocked in 50% Chemiblocker (Millipore cat #2170)/50% 1×TBST (Teknova cat #T0310) for one hour. Libraries were added to the SA beads to adsorb clones that bind non-specifically to the beads. SA beads were discarded and the pre-cleared library was added to biotinylated human IL-23R ECD. Binders were retrieved by addition of SA beads to form a bead/antigen/phage complex, which was washed in 1×TBST. After the final wash, phage was rescued by infection of log phase TG1 E. coli cells (OD600 nm=0.4-0.6). The phage-infected TG1 cells were spread on three 150 mm LB agar plates containing 75 μg/ml carbenicillin and 1% glucose and grown overnight at 37° C. Phage was produced and subjected to additional panning. To increase selection pressure, the antigen concentration was reduced for each subsequent round: R1 100 nM, R2 50 nM, R3 10 nM, R4 10 nM, R1-R4 all for 1 hour at room temperature; R5 5 nM, overnight at 4° C.; R6 2.5 nM, overnight at 4° C.

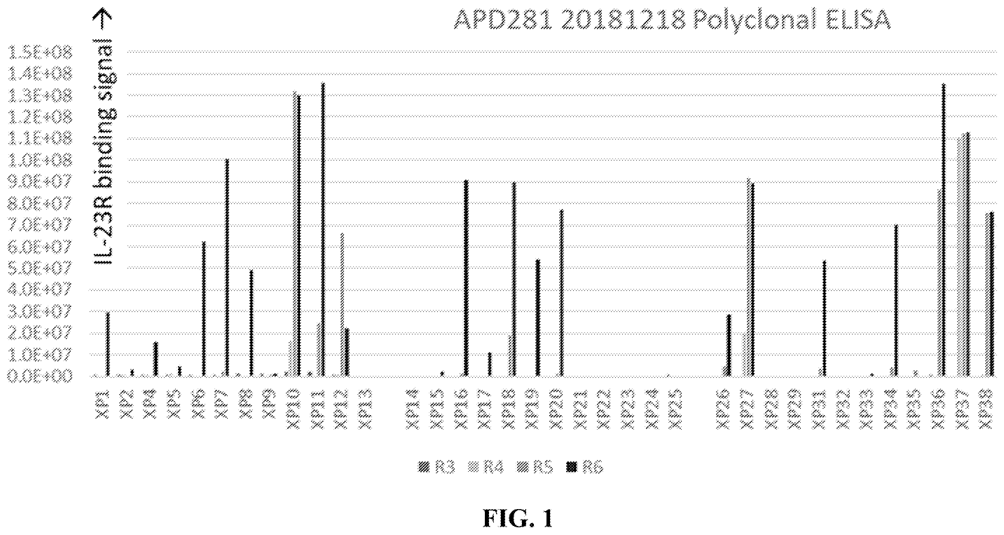

After multiple selection rounds, a polyclonal phage ELISA using human IL-23R ECD was performed to determine the enrichment of antigen-specific binders in individual panning experiments (representative polyclonal phage ELISA in FIG. 1).

Polyclonalphage ELISA. Briefly, 100 μl of 20 nM biotinylated human IL-23R ECD diluted in 1×TBS (Teknova cat #T9530) was captured on an NA-coated plate (Thermo cat #15217). After an hour incubation at 37° C., the plate was washed 3 times in 300 μl 1×TBST per well. 300 μl of blocking buffer 50% Chemiblocker/50% 1×TBST was added to each well of the plate and incubated at room temperature for 1 hour. After blocking, the plate was washed 3 times with 300 μl of 1×TBST per well. Polyclonal phage output for each panning round of each panning experiment was diluted 1/100 diluted in assay buffer (10% Chemiblocker/90% 1×TBST). 100 μL of these diluted phage outputs was added to the blocked ELISA plate and incubated at room temperature for 1 hour to allow the binding of Fab displayed on the phage particles to the immobilized human IL-23R ECD. Following the incubation, the plate was washed 3 times with 1×TBST. 100 μl of HRP-conjugated anti-M13 (pVIII) antibody (GE Healthcare cat #27942101) diluted 1:2500 in assay buffer was added to the plate and incubated at room temperature. After 1 hour incubation, the plate was washed 6 times with 300 μl of 1×TBST per well. 100 μL of prepared BM chemiluminescence ELISA substrate (Roche cat #11582950001) was added to the plate. The chemiluminescence or relative light unit (RLU) was measured in an Envision plate reader.

Phage collected from panning experiments which demonstrated enrichment for human IL-23R ECD binders, were used to express monoclonal Fab proteins in E. coli for primary screening.

Fab production. Briefly, plasmid DNA was isolated and purified from glycerol stocks of specific rounds of phage panning experiments that were identified as demonstrating enrichment of binders to human IL-23 receptor and transformed into TG-1 E. coli cells and grown on LB agar plates overnight. The plates of transformants were used for (i) colony PCR and sequencing of the V-regions and (ii) starter cultures for Fab production. For Fab production, the overnight cultures were diluted 10-100 fold in new media, grown for 5-6 hours at 37° C. and Fab production induced by the addition of fresh media containing IPTG. Induced cultures were grown overnight at 30° C. The cultures were spun down and the bacterial pellet was lysed using BugBuster™ (Millipore) to release the soluble Fab proteins. The cell lysates were cleared by centrifugation and the supernatants were used for Fab ELISA.

Monoclonal Fab lysates that were prepared from the enriched Fab libraries were screened by ELISA for binding to human IL-23 receptor and lack of binding to a negative control antigen, mouse IL-6.

Monoclonal Fab screening. Black maxisorp plates (ThermoFisher Scientific, Cat #437111) were coated with 100 μl of 10 ng/μl goat anti-mouse Fab2 (Jackson Immuno Research, Code #115-005-072) or 4 ng/μl of sheep anti-human Fd (The Binding Site Group, Product Code AU075). After overnight incubation at 4° C., the coated plates were blocked with 250p1 of Chemiblocker (Milipore, Cat #2170) and incubated at room temperature for 1 hour. 100 μl of each Fab sample were added to wells and the plates were incubated at room temperature for 1 hour with shaking at 20 rpm, followed by 5 washes with 1×TBST. 100 μl of 20 nM biotinylated human IL-23 receptor ECD or murine IL-6 (counter screening) were added to wells and incubated at room temperature for 1 hour with shaking 20 rpm, followed by 5 washes with 1×TBST. 100 μl of 1:5000 strepavidin HRP were added, and the plates incubated at room temperature for 40 minutes, followed by 3 washes with 1×TBST. 100 μL per well of prepared BM chemiluminescence ELISA substrate (Roche cat #11582950001) was added to the plate. The chemiluminescence or relative light unit (RLU) was measured in an Envision plate reader.

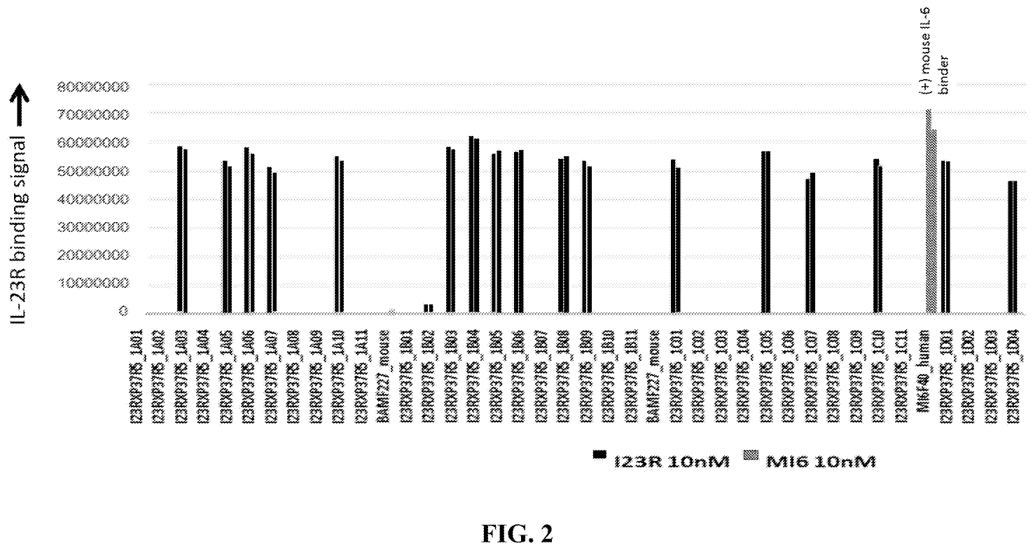

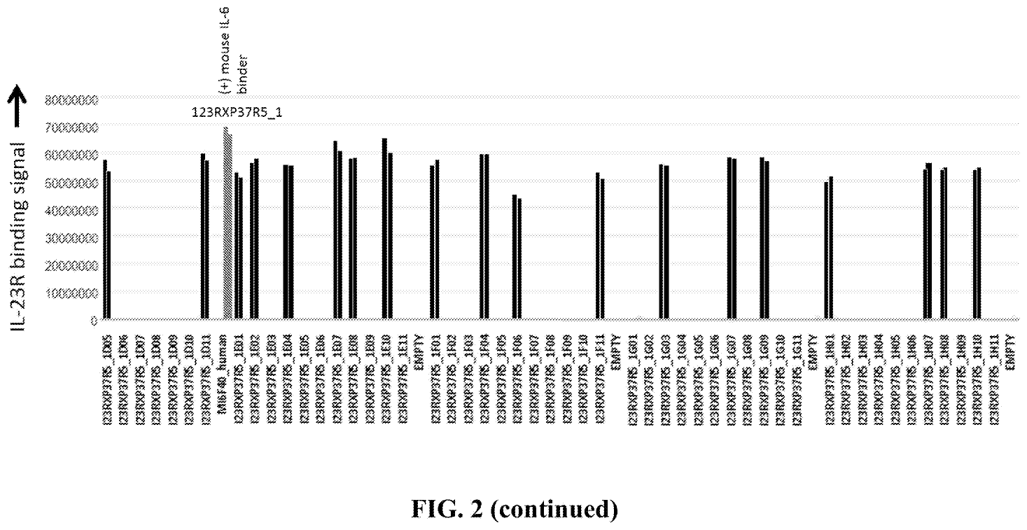

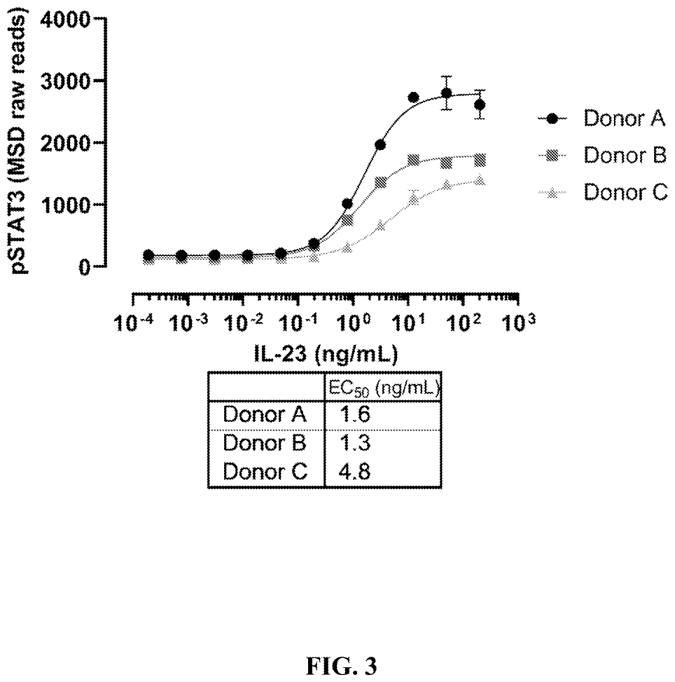

Fab clones meeting the selection criteria of binding to human IL-23 receptor and not binding to a negative control antigen, mouse IL-6, were sequenced to identify the unique Fabs (representative monoclonal Fab ELISA in FIG. 2).

Heavy chain (HC) and light chain (LC) variable (V) regions (VH and VL, respectively) of the unique Fab were cloned into mammalian expression vectors to express recombinant mAbs with murine IgG₂ₐ/murine Kappa constant regions. Plasmid DNA was prepared for all expression constructs generated for use in various scales of expression of the recombinant antibodies.

Cloning VH and VL of IL-23R binding Fab hits. Two pcDNA3.1 derived mammalian expression vectors (vDR000368 and vDR000961) were used to generate the single gene constructs encoding a heavy chain (HC) or light chain (LC) for each Fab hit. Each vector contains a human cytomegalovirus (hCMV) promoter to drive the expression of the HC and LC and both contain the ampicillin resistance gene (Amp(R)) to facilitate cloning. vDR000368 has unique HindIII and DraIII restriction enzyme sites for VH cloning and also a mouse IgG₂ₐ constant region; vDR000961 has unique HindIII and Tth111I restriction enzyme sites for VL cloning and also a mouse Kappa constant region.

### IL-23R mAb Expression and Purification

Recombinant IL-23R binding mAbs discovered from both the immunization and phage display strategies were transiently expressed in either ExpiCHO or HEK Expi293™ cells in various expression volumes from 2 ml to 500 ml using prepared and quantitated plasmid DNA of the heavy chain (HC) and light chain (LC) mammalian expression constructs generated for these mAbs.

Expression in transiently transfected HEK Expi293™ cells. HEK Expi293™ cells (Thermo cat #A14527) were grown in Expi293™ expression media (Thermo cat #A1435101) at 37° C. shaking at 125RPM with 8% CO₂. The cells were transfected at 2.5×10⁶ cells per ml using Expi293™ Expression Kit (Thermo cat #A14524). For each liter of cells transfected, 1 mg of total DNA was diluted in 25 ml of Opti-MEM (Thermo cat #319850620) and 2.6 ml of Expi293™ reagent was diluted in 25 ml of Opti-MEM and incubated for 5 minutes at room temperature. The diluted DNA and diluted Expi293 reagent were combined and incubated for 20 minutes at room temperature. The DNA complex was then added to the cells and the cells placed in the shaking incubator overnight. The next day after transfection, 5 ml of Enhancer 1 was diluted into 50 ml of Enhancer 2 and the total volume of the two Enhancers was added to the cells. The transfected cells were placed back into the incubator for 4 days until harvested. The cells were removed by centrifugation at 4,500 g for 35 minutes then the cleared culture is filtered with a 0.2 μm filter prior to checking expression levels. This protocol can be used with varying expression volumes by adjusting reagents in a linear manner. When transfection is done with 96-well plates incubation is done in an incubator with a 3 mm shaking diameter set at 1,000 RPM. Expression was quantitated by Octet using Protein A biosensors. Murine IgG2 (Sigma Cat #M9144) was used as the standard. The samples and the standard were diluted with spent Expi293 media. The standard curve was generated with two-fold dilutions starting at 100 μg/ml, using the linear portion. The samples were diluted 1:10 for measurement. The calculations were performed by the Forte Biosystems software.

Expression in transiently transfected ExpiCHO cells. Transient transfection using ExpiCHO-S™ cells (ThermoFisher Scientific, Cat #A29127) with purified DNA plasmid following manufacturer's recommendations. Briefly, ExpiCHO-S™ cells were maintained in suspension in ExpiCHO™ expression medium (ThermoFisher Scientific, Cat #A29100) in a shaking incubator set at 37° C., 8% CO₂ and 125 RPM. The cells were passaged so that on the day of transfection, dilution down to 6.0×10⁶ cells per ml could be achieved, maintaining cell viability at 98% or better. Transient transfections were done using the ExpiFectamine™ CHO transfection kit (ThermoFisher Scientific Cat #A29131). For each ml of diluted cells to be transfected, one microgram of plasmid DNA is used and diluted into OptiPRO™ SFM complexation medium. ExpiFectamine™ CHO reagent is used at a 1:3 ratio (v/v, DNA:reagent) and also diluted into OptiPRO™. The diluted DNA and transfection reagent were combined for one minute, allowing DNA/lipid complex formation, and then added to the cells. After overnight incubation, ExpiCHO™ feed and ExpiFectamine™ CHO enhancer were added to the cells. Cells were cultured with shaking at 32° C. for five days prior to harvesting the culture supernatants. Culture supernatants from the transiently transfected ExpiCHO-S™ cells were harvested by clarifying through centrifugation (35 min, 6,500 rpm) followed by filtration (0.24 PES membrane, Corning).

The crude mammalian expression supernatants, after quantitation of mAb concentration, were used directly for some characterization of the unpurified mAbs contained within. Some IL-23R mAbs were purified using a routine Protein A purification protocol and characterized as purified mAbs.

Protein A purification of mAbs expressed in transient transfections. Pilot scale (0.3-3 mgs) purifications were performed on the AKTA Xpress chromatography system and large-scale (3 mgs or greater) purifications were performed using the AKTA FPLC chromatography systems. The purification procedures for pilot and large-scale preps were identical. Briefly, harvested, clarified and filtered supernatants from transiently transfected mammalian cells were loaded onto an equilibrated (PBS, pH 7.2) HiTrap MabSelect Sure Protein A column (GE Healthcare) at a relative concentration of ˜30 mg protein per ml of resin. After loading, the column was washed with PBS, pH7.2 and protein eluted with 10 column volumes of 0.1 M Na-Acetate, pH 3.5. The protein fractions were neutralized immediately by elution into tubes containing 2.0 M Tris, pH 7 at 20% the elution fraction volume. Peak fractions were pooled, further neutralized, if necessary, filtered (0.2 μm) and dialyzed against 3 changes of PBS, pH 7.2 overnight at 4° C. The next day, samples were removed from dialysis, filtered (0.2 μm) and the protein concentration determined by absorbance at 280 nm. The quality of the purified proteins was assessed by SDS-PAGE and analytical size exclusion HPLC (Dionex HPLC system). Endotoxin levels were measured using a LAL assay (Pyrotell®-T, Associates of Cape Cod). Purified proteins were stored at 4° C.

Exemplary IL-23R mAbs

Examples of immunization based IL-23R antibodies or antigen binding fragments of the present disclosure are summarized in Table 4, Table 5 and Table 6:

TABLE 4
HC and LC AA Sequences of Exemplary Immunization Based IL-23R Antibodies

| Name | HC AA Seq | LC AA Seq |
|---|---|---|
| I23RB101 | EVQLQQSGAEVKKPGSSVKVSCKA | DIVMTQSHKFMSTSIGDRVSITCKTSQDV |
| | SGGTFSSYAISWVRQAPGQGLEWM | RTAVAWYQQKPGQSPKLLIYSASYRYIG |
| | GWINPNSGGTNYAQKFQGRVTMTR | VPDRFTGSGSGTDFTFTISDVQAEDLALY |
| | DTSISTAYMELSRLRSDDTAVYYCA | YCQQHYSTPYTFGGGTKLEIKRADAAPT |
| | RGYRYGPLDGFDIWGQGTTLTVSSA | VSIFPPSSEQLTSGGASVVCFLNNFYPKDI |
| | KTTAPSVYPLAPVCGDTTGSSVTLG | NVKWKIDGSERQNGVLNSWTDQDSKDS |
| | CLVKGYFPEPVTLTWNSGSLSSGVH | TYSMSSTLTLTKDEYERHNSYTCEATHKT |
| | TFPAVLQSDLYTLSSSVTVTSSTWPS | STSPIVKSFNRNEC (SEQ ID NO: 3) |
| | QSITCNVAHPASSTKVDKKIEPRGPT | |
| | IKPCPPCKCPAPNLLGGPSVFIFPPKI | |
| | KDVLMISLSPIVTCVVVDVSEDDPD | |
| | VQISWFVNNVEVHTAQTQTHREDY | |
| | NSTLRVVSALPIQHQDWMSGKEFK | |
| | CKVNNKDLPAPIERTISKPKGSVRAP | |
| | QVYVLPPPEEEMTKKQVTLTCMVT | |
| | DFMPEDIYVEWTNNGKTELNYKNT | |
| | EPVLDSDGSYFMYSKLRVEKKNWV | |
| | ERNSYSCSVVHEGLHNHHTTKSFSR | |
| | TPGK (SEQ ID NO: 2) | |
| I23RB102 | QVQLQQPGAELVKPGASVKVSCKA | DIQMTQSPASLSVSVGETVTITCRASENIY |
| | SGYTFTKYWINWMNQRPGQGLEWI | SHITWYQLKQGKSPQLLVYASTNLADDV |
| | GNIYPGSISTNYNEKFKSKVTLTVDI | PSRFSGSGSGTQYSLKINSLLSEDFGTYYC |
| | SSSTAYMQLSSLTSDDSAVYYCARG | QHFWGTPWTFGGGTKLEMKRADAAPTV |
| | DFYALDYWGQGTTLTVSSAKTTAP | SIFPPSSEQLTSGGASVVCFLNNFYPKDIN |
| | SVYPLAPVCGDTTGSSVTLGCLVKG | VKWKIDGSERQNGVLNSWTDQDSKDST |
| | YFPEPVTLTWNSGSLSSGVHTFPAV | YSMSSTLTLTKDEYERHNSYTCEATHKTS |
| | LQSDLYTLSSSVTVTSSTWPSQSITC | TSPIVKSFNRNEC (SEQ ID NO: 5) |
| | NVAHPASSTKVDKKIEPRGPTIKPCP | |
| | PCKCPAPNLLGGPSVFIFPPKIKDVL | |
| | MISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 4) | |
| I23RB104 | EVKLVESGGGLVKPGGSLKLSCAAS | DIQMTQSPASLSVSVGETVTITCRASENIY |
| | GFTFSSYAMSWVRQTPEKRLDWVA | SNLAWYQQKQGKSPQLLVYAATNLADG |
| | SISTGGSTYYPDSVKGGFTISRDIAR | VPSRFSGSGSGTQYSLKINSLQSEDFGSYY |
| | NTLSLQMTSLRSEDTAMYYCARGP | CQHFWGTPPTFGSGTKLEIKRADAAPTVS |
| | HYYGGSYPFAYWGQGTTLTVSSAK | IFPPSSEQLTSGGASVVCFLNNFYPKDINV |
| | TTAPSVYPLAPVCGDTTGSSVTLGC | KWKIDGSERQNGVLNSWTDQDSKDSTYS |
| | LVKGYFPEPVTLTWNSGSLSSGVHT | MSSTLTLTKDEYERHNSYTCEATHKTSTS |
| | FPAVLQSDLYTLSSSVTVTSSTWPSQ | PIVKSFNRNEC (SEQ ID NO: 7) |
| | SITCNVAHPASSTKVDKKIEPRGPTI | |
| | KPCPPCKCPAPNLLGGPSVFIFPPKIK | |
| | DVLMISLSPIVTCVVVDVSEDDPDV | |
| | QISWFVNNVEVHTAQTQTHREDYN | |
| | STLRVVSALPIQHQDWMSGKEFKC | |
| | KVNNKDLPAPIERTISKPKGSVRAPQ | |
| | VYVLPPPEEEMTKKQVTLTCMVTD | |
| | FMPEDIYVEWTNNGKTELNYKNTE | |
| | PVLDSDGSYFMYSKLRVEKKNWVE | |
| | RNSYSCSVVHEGLHNHHTTKSFSRT | |
| | PGK (SEQ ID NO: 6) | |
| I23RB144 | EVQLQQSGPELVKPGASVKMSCKA | DIVMTQSPLTLSVIIGQSASISCKSSQSLLD |
| | SGYTFTSYIIHWVKQKPGQGLEWIG | SDGKTYLNWLLQRPGQSPKRLMYLVSKL |
| | YIDPYNDDTKYNEKFKGKATLTSD | DSGVPDRFTGSGSGTDFTLKISRVEAEDL |
| | KSSSTAYMEFSSLTSEDSAVYYCAR | GVYYCWQGIHFPLTFGSGTRLEIKRADAA |
| | LGSINYYDGSYFDYWGQGTSVTVSS | PTVSIFPPSSEQLTSGGASVVCFLNNFYPK |
| | AKTTAPSVYPLAPVCGDTTGSSVTL | DINVKWKIDGSERQNGVLNSWTDQDSKD |
| | GCLVKGYFPEPVTLTWNSGSLSSGV | STYSMSSTLTLTKDEYERHNSYTCEATHK |
| | HTFPAVLQSDLYTLSSSVTVTSSTW | TSTSPIVKSFNRNEC (SEQ ID NO: 9) |
| | PSQSITCNVAHPASSTKVDKKIEPRG | |
| | PTIKPCPPCKCPAPNLLGGPSVFIFPP | |
| | KIKDVLMISLSPIVTCVVVDVSEDDP | |
| | DVQISWFVNNVEVHTAQTQTHRED | |
| | YNSTLRVVSALPIQHQDWMSGKEF | |
| | KCKVNNKDLPAPIERTISKPKGSVR | |
| | APQVYVLPPPEEEMTKKQVTLTCM | |
| | VTDFMPEDIYVEWTNNGKTELNYK | |
| | NTEPVLDSDGSYFMYSKLRVEKKN | |
| | WVERNSYSCSVVHEGLHNHHTTKS | |
| | FSRTPGK (SEQ ID NO: 8) | |
| I23RB148 | DVQLQESGPDLVKPSQSLSLTCTVT | DIVMTQSHKFMSTSVGDRVSLTCKASQE |
| | GYSITSGYNWHWIRQFPGNKLEWM | VRTAVAWYQQKPGQPPKLLIYSASYRYT |
| | GYIHSSGTTNYSPSLRSRLSITRDTSK | GVPDRFTGSGSGTDFTFTIISVQAEDLAVY |
| | NQFFLQLNSVTTEDTATYYCTRQLG | YCQQHYTTPYSFGSGTKLEIKRADAAPTV |
| | LKAMEYWGQGTTLTVSSAKTTAPS | SIFPPSSEQLTSGGASVVCFLNNFYPKDIN |
| | VYPLAPVCGDTTGSSVTLGCLVKG | VKWKIDGSERQNGVLNSWTDQDSKDST |
| | YFPEPVTLTWNSGSLSSGVHTFPAV | YSMSSTLTLTKDEYERHNSYTCEATHKTS |
| | LQSDLYTLSSSVTVTSSTWPSQSITC | TSPIVKSFNRNEC (SEQ ID NO: 11) |
| | NVAHPASSTKVDKKIEPRGPTIKPCP | |
| | PCKCPAPNLLGGPSVFIFPPKIKDVL | |
| | MISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 10) | |
| I23RB149 | DVQLQESGPDLVKPSQSLSLTCTVT | DVQMIQSPLSLPVSLGDQASISCRSSQSLV |
| | GYSITSGYNWHWIRQFPGNKLEWM | HSNGNTYLHWYLQKPGQSPKLLIYKVSN |
| | GYIHFSDRTNYNPSLKSRISITRDTSK | RFSGVPDRFSGSGSGTDFTLKISRVEAEDL |
| | NQFFLQLNSVTSEDTATYYCSRQLG | GVYFCSQSTHVPLTFGAGTRLEIKRADAA |
| | LKALDYWGQGTTLTVSSAKTTAPS | PTVSIFPPSSEQLTSGGASVVCFLNNFYPK |
| | VYPLAPVCGDTTGSSVTLGCLVKG | DINVKWKIDGSERQNGVLNSWTDQDSKD |
| | YFPEPVTLTWNSGSLSSGVHTFPAV | STYSMSSTLTLTKDEYERHNSYTCEATHK |
| | LQSDLYTLSSSVTVTSSTWPSQSITC | TSTSPIVKSFNRNEC (SEQ ID NO: 13) |
| | NVAHPASSTKVDKKIEPRGPTIKPCP | |
| | PCKCPAPNLLGGPSVFIFPPKIKDVL | |
| | MISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 12) | |
| I23RB150 | QIQLVQSGPELKKPGETVKISCKASA | DIVMTQSHKFMSTSVGGRVSITCKASQD |
| | YTFTNYGMNWVKQAPGKGLKWM | VSTAVAWYQQKPGQSPRLLIYSASYRYT |
| | GWIKTNTGEPTYAEEFKGRFAFSLE | GVPDRFTGSGSGTDFTFTISSVQAEDLAV |
| | TSASTAYLQINNLKNEDTATYFCAR | FYCQQHYSTPYTFGSGTRLEIKRADAAPT |
| | SRQLGLRYYFDYWGQGTTLTVSSA | VSIFPPSSEQLTSGGASVVCFLNNFYPKDI |
| | KTTAPSVYPLAPVCGDTTGSSVTLG | NVKWKIDGSERQNGVLNSWTDQDSKDS |
| | CLVKGYFPEPVTLTWNSGSLSSGVH | TYSMSSTLTLTKDEYERHNSYTCEATHKT |
| | TFPAVLQSDLYTLSSSVTVTSSTWPS | STSPIVKSFNRNEC (SEQ ID NO: 15) |
| | QSITCNVAHPASSTKVDKKIEPRGPT | |
| | IKPCPPCKCPAPNLLGGPSVFIFPPKI | |
| | KDVLMISLSPIVTCVVVDVSEDDPD | |
| | VQISWFVNNVEVHTAQTQTHREDY | |
| | NSTLRVVSALPIQHQDWMSGKEFK | |
| | CKVNNKDLPAPIERTISKPKGSVRAP | |
| | QVYVLPPPEEEMTKKQVTLTCMVT | |
| | DFMPEDIYVEWTNNGKTELNYKNT | |
| | EPVLDSDGSYFMYSKLRVEKKNWV | |
| | ERNSYSCSVVHEGLHNHHTTKSFSR | |
| | TPGK (SEQ ID NO: 14) | |
| I23RB153 | EVKLVESGGGLVQPGGSLKLSCAAS | DIVMTQSHRFMSTSVGERVSITCKASQDV |
| | GFTFSSDTMSWVRQTPEKRLELVAE | GNAVAWYQQKPGQSPKLLIYWTSTRHN |
| | ISSNGGSTYHIDTVKGRFTISRDNAK | GVPDRFTGSGSGTDFTLTISNVQGEDLAD |
| | NTLYLQMSSLKSEDTAMYYCASPG | YFCQQYNRYPLTFGSGTKLEIKRADAAPT |
| | YPWFAYWGQGTTVTVSAAKTTAPS | VSIFPPSSEQLTSGGASVVCFLNNFYPKDI |
| | VYPLAPVCGDTTGSSVTLGCLVKG | NVKWKIDGSERQNGVLNSWTDQDSKDS |
| | YFPEPVTLTWNSGSLSSGVHTFPAV | TYSMSSTLTLTKDEYERHNSYTCEATHKT |
| | LQSDLYTLSSSVTVTSSTWPSQSITC | STSPIVKSFNRNEC (SEQ ID NO: 17) |
| | NVAHPASSTKVDKKIEPRGPTIKPCP | |
| | PCKCPAPNLLGGPSVFIFPPKIKDVL | |
| | MISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 16) | |
| I23RB155 | QVQLKESGPGLVKPSQSLSLTCSVT | QIVLSQSPASLAVSLGQRATISCRTSESVD |
| | GYSITSGHSWNWIRQFPGNKLEWM | TYGNSFIYWYQQKPGQPPKLLIYRASNLE |
| | GYISYDGTNDYNPSLKNRISITRDTS | SGIPARFSGSGSRTDFTLTINPVEADDVAT |
| | KNQFFLNLNSVTTEDTVTYYCARG | YYCQQGNKDPYTFGSGTKLEMKRADAA |
| | GGYYGGTYRVFADWGQGTTLTVSS | PTVSIFPPSSEQLTSGGASVVCFLNNFYPK |
| | AKTTAPSVYPLAPVCGDTTGSSVTL | DINVKWKIDGSERQNGVLNSWTDQDSKD |
| | GCLVKGYFPEPVTLTWNSGSLSSGV | STYSMSSTLTLTKDEYERHNSYTCEATHK |
| | HTFPAVLQSDLYTLSSSVTVTSSTW | TSTSPIVKSFNRNEC (SEQ ID NO: 19) |
| | PSQSITCNVAHPASSTKVDKKIEPRG | |
| | PTIKPCPPCKCPAPNLLGGPSVFIFPP | |
| | KIKDVLMISLSPIVTCVVVDVSEDDP | |
| | DVQISWFVNNVEVHTAQTQTHRED | |
| | YNSTLRVVSALPIQHQDWMSGKEF | |
| | KCKVNNKDLPAPIERTISKPKGSVR | |
| | APQVYVLPPPEEEMTKKQVTLTCM | |
| | VTDFMPEDIYVEWTNNGKTELNYK | |
| | NTEPVLDSDGSYFMYSKLRVEKKN | |
| | WVERNSYSCSVVHEGLHNHHTTKS | |
| | FSRTPGK (SEQ ID NO: 18) | |
| I23RB156 | QVQLKESGPGLVAPSQSLSITCTVSG | DIVMTQSHKFMSTSVGDRVSITCKASQD |
| | FSLTSYAINWVRQPPGKGLEWLGVI | VSTAVAWYQQKPGRSPKLLIYSAFYRYT |
| | WTGGGTNYNSVFKSRLSISKDNSKS | GVPDRFTGSGSGTDFTFTIGSVQAEDLAV |
| | QIFLKMISLQTDDTARYYCARNRDY | YYCQQHYSIPLTFGAGTKLELKRADAAPT |
| | SNYRWYFDVWGAGTTLTVSSAKTT | VSIFPPSSEQLTSGGASVVCFLNNFYPKDI |
| | APSVYPLAPVCGDTTGSSVTLGCLV | NVKWKIDGSERQNGVLNSWTDQDSKDS |
| | KGYFPEPVTLTWNSGSLSSGVHTFP | TYSMSSTLTLTKDEYERHNSYTCEATHKT |
| | AVLQSDLYTLSSSVTVTSSTWPSQSI | STSPIVKSFNRNEC (SEQ ID NO: 21) |
| | TCNVAHPASSTKVDKKIEPRGPTIKP | |
| | CPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 20) | |
| I23RB157 | EVKLVESGGGSVKPGGSLKLSCAAS | DIVMTQSPASLAVSLGQRATISCRASESV |
| | GFTFSRYTMSWVRQTPEKRLEWVA | DNYGISFMNWFQQKPGQPPKLLIYAASN |
| | YISTGGGDTYYPDSVKGRFTISRDN | QGSGVPARFSGSGSGTDFSLNIHPMEEDD |
| | ARNTLYLQMSSLRSEDTAMYYCAR | TAMYFCQQSKEVPWTFGAGTKLELKRA |
| | RTSYSYYNYVFDYWGQGTTLTVSS | DAAPTVSIFPPSSEQLTSGGASVVCFLNNF |
| | AKTTAPSVYPLAPVCGDTTGSSVTL | YPKDINVKWKIDGSERQNGVLNSWTDQD |
| | GCLVKGYFPEPVTLTWNSGSLSSGV | SKDSTYSMSSTLTLTKDEYERHNSYTCEA |
| | HTFPAVLQSDLYTLSSSVTVTSSTW | THKTSTSPIVKSFNRNEC (SEQ ID NO: 23) |
| | PSQSITCNVAHPASSTKVDKKIEPRG | |
| | PTIKPCPPCKCPAPNLLGGPSVFIFPP | |
| | KIKDVLMISLSPIVTCVVVDVSEDDP | |
| | DVQISWFVNNVEVHTAQTQTHRED | |
| | YNSTLRVVSALPIQHQDWMSGKEF | |
| | KCKVNNKDLPAPIERTISKPKGSVR | |
| | APQVYVLPPPEEEMTKKQVTLTCM | |
| | VTDFMPEDIYVEWTNNGKTELNYK | |
| | NTEPVLDSDGSYFMYSKLRVEKKN | |
| | WVERNSYSCSVVHEGLHNHHTTKS | |
| | FSRTPGK (SEQ ID NO: 22) | |
| I23RB159 | EVQLQQSGGGLVKPGGSLKLSCAA | DIVMTQTTSSLSASLGDRVTISCRASQDIS |
| | SGFTFSDYGMAWVRQTPRKGPEWI | NYLNWYQQKPDGTVKLLIYYTSRLHSGV |
| | AFISNLAYSVYYSDTVTGRFTISREN | PSRFSGSGSGTDYSLTISNLEQEDIATYFC |
| | AKNTLYLEMSSLRSEDTAMYYCAR | QQGNTLPWTFGAGTKLELKRADAAPTVS |
| | HGDYRNYGFAYWGLGTTVTVSSAK | IFPPSSEQLTSGGASVVCFLNNFYPKDINV |
| | TTAPSVYPLAPVCGDTTGSSVTLGC | KWKIDGSERQNGVLNSWTDQDSKDSTYS |
| | LVKGYFPEPVTLTWNSGSLSSGVHT | MSSTLTLTKDEYERHNSYTCEATHKTSTS |
| | FPAVLQSDLYTLSSSVTVTSSTWPSQ | PIVKSFNRNEC (SEQ ID NO: 25) |
| | SITCNVAHPASSTKVDKKIEPRGPTI | |
| | KPCPPCKCPAPNLLGGPSVFIFPPKIK | |
| | DVLMISLSPIVTCVVVDVSEDDPDV | |
| | QISWFVNNVEVHTAQTQTHREDYN | |
| | STLRVVSALPIQHQDWMSGKEFKC | |
| | KVNNKDLPAPIERTISKPKGSVRAPQ | |
| | VYVLPPPEEEMTKKQVTLTCMVTD | |
| | FMPEDIYVEWTNNGKTELNYKNTE | |
| | PVLDSDGSYFMYSKLRVEKKNWVE | |
| | RNSYSCSVVHEGLHNHHTTKSFSRT | |
| | PGK (SEQ ID NO: 24) | |
| I23RB160 | QVQLKESGPDLVKPSQSLSLTCTVT | DIVMTQSHKFMSTSVGDRVSITCKASQD |
| | GYSITSGYNWHWIRQFPGNKLEWM | VRTAVAWYQQKPGQSPKLLIHSASYRYT |
| | GYIHYSSGTIYNPSLKSRISITRDTSK | GVPDRFTGSGSGTDFTFTISSVQVEDLAV |
| | NQFFLQLNSVTTEDTGTYYCARQLG | YYCQQHYTTPYTFGSGTKLEIKRADAAPT |
| | LKAMDYWGQGTSVTVSSAKTTAPS | VSIFPPSSEQLTSGGASVVCFLNNFYPKDI |
| | VYPLAPVCGDTTGSSVTLGCLVKG | NVKWKIDGSERQNGVLNSWTDQDSKDS |
| | YFPEPVTLTWNSGSLSSGVHTFPAV | TYSMSSTLTLTKDEYERHNSYTCEATHKT |
| | LQSDLYTLSSSVTVTSSTWPSQSITC | STSPIVKSFNRNEC (SEQ ID NO: 27) |
| | NVAHPASSTKVDKKIEPRGPTIKPCP | |
| | PCKCPAPNLLGGPSVFIFPPKIKDVL | |
| | MISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 26) | |
| I23RB161 | EFQLQQSGPELVKPGASVKISCKAS | DIVMTQSPAILSASPGEKVTMTCRASSSV |
| | VYAFSSSWMNWVKQRPGKGLEWI | SYMHWYQQKPGSSPKPWIYATSDLASGA |
| | GRIYPGDGDTNYNGKFKGKATLTA | PARFSGSGSGTSYSLTISRVEAEDAATYY |
| | DKSSSTAYMQLSSLTSEDSAVYFCA | CQQWSSNPPTFGGGTKLEIKRADAAPTVS |
| | RRYDFGSAMDYWGQGTSVTVSSAK | IFPPSSEQLTSGGASVVCFLNNFYPKDINV |
| | TTAPSVYPLAPVCGDTTGSSVTLGC | KWKIDGSERQNGVLNSWTDQDSKDSTYS |
| | LVKGYFPEPVTLTWNSGSLSSGVHT | MSSTLTLTKDEYERHNSYTCEATHKTSTS |
| | FPAVLQSDLYTLSSSVTVTSSTWPSQ | PIVKSFNRNEC (SEQ ID NO: 29) |
| | SITCNVAHPASSTKVDKKIEPRGPTI | |
| | KPCPPCKCPAPNLLGGPSVFIFPPKIK | |
| | DVLMISLSPIVTCVVVDVSEDDPDV | |
| | QISWFVNNVEVHTAQTQTHREDYN | |
| | STLRVVSALPIQHQDWMSGKEFKC | |
| | KVNNKDLPAPIERTISKPKGSVRAPQ | |
| | VYVLPPPEEEMTKKQVTLTCMVTD | |
| | FMPEDIYVEWTNNGKTELNYKNTE | |
| | PVLDSDGSYFMYSKLRVEKKNWVE | |
| | RNSYSCSVVHEGLHNHHTTKSFSRT | |
| | PGK (SEQ ID NO: 28) | |
| I23RB162 | EFQLQQSGPELVKPGASVKMSCKAS | DIVMTQSQKFMSTSVGDRVSVTCKASQN |
| | GYTFTNYVMHWVKQKPGQGLEWI | VGTNVAWYQQKPGQSPKALIYSASYRYS |
| | GYINPYNDGTKYNEKFKGKATLTS | GVPDRFTGSGSGTDFTLTISNVQSEDLAE |
| | DKSSSTAYMELSSLTSEDSAVYYCA | YFCQQYNSYPFTFGSGTKLEIKRADAAPT |
| | RSRTGTRWYFDVWGAGTTVTVSSA | VSIFPPSSEQLTSGGASVVCFLNNFYPKDI |
| | KTTAPSVYPLAPVCGDTTGSSVTLG | NVKWKIDGSERQNGVLNSWTDQDSKDS |
| | CLVKGYFPEPVTLTWNSGSLSSGVH | TYSMSSTLTLTKDEYERHNSYTCEATHKT |
| | TFPAVLQSDLYTLSSSVTVTSSTWPS | STSPIVKSFNRNEC (SEQ ID NO: 31) |
| | QSITCNVAHPASSTKVDKKIEPRGPT | |
| | IKPCPPCKCPAPNLLGGPSVFIFPPKI | |
| | KDVLMISLSPIVTCVVVDVSEDDPD | |
| | VQISWFVNNVEVHTAQTQTHREDY | |
| | NSTLRVVSALPIQHQDWMSGKEFK | |
| | CKVNNKDLPAPIERTISKPKGSVRAP | |
| | QVYVLPPPEEEMTKKQVTLTCMVT | |
| | DFMPEDIYVEWTNNGKTELNYKNT | |
| | EPVLDSDGSYFMYSKLRVEKKNWV | |
| | ERNSYSCSVVHEGLHNHHTTKSFSR | |
| | TPGK (SEQ ID NO: 30) | |
| I23RB165 | EVQLQQSGAELVRPGASVKLSCTAS | DIVMTQSPASLAVSLGQRATISCRASKSV |
| | GFNIKDSLMHWVKQRPEQGLEWIG | STSGYSYMHWYQQKPGQPPKLLIYLASN |
| | WIDPEDGKTKYAPKFQDKATITADT | LESGVPARFSGSGSGTDFTLNIHPVEEEDA |
| | SSNTAYLQLSSLTSEDTAIYYCARRE | ATYYCQHSRELPLTFGAGTKLEMKRADA |
| | VLDYFDYWGQGTTLTVSSAKTTAP | APTVSIFPPSSEQLTSGGASVVCFLNNFYP |
| | SVYPLAPVCGDTTGSSVTLGCLVKG | KDINVKWKIDGSERQNGVLNSWTDQDSK |
| | YFPEPVTLTWNSGSLSSGVHTFPAV | DSTYSMSSTLTLTKDEYERHNSYTCEATH |
| | LQSDLYTLSSSVTVTSSTWPSQSITC | KTSTSPIVKSFNRNEC (SEQ ID NO: 33) |
| | NVAHPASSTKVDKKIEPRGPTIKPCP | |
| | PCKCPAPNLLGGPSVFIFPPKIKDVL | |
| | MISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 32) | |
| I23RB166 | DVQLQESGPGLVKPSQTVSLTCTVT | DIVMTQSHKFMSTSVGARVSITCKASQD |
| | GYSITNGNHWWNWIRQVSGSKLEW | VSTAVAWYQQKPGQSPKLLIYSASYRYT |
| | IGYIRSSGSTDSNPSLKSRISITRDTSK | GVPDRFTGSGSGTDFTFTISSVQAEDLAV |
| | NQLFLQLNSVTAEDIATYYCARERL | YYCQQHYSTPRTFGGGTKLEIKRADAAP |
| | RLRWSYFDYWGQGTTLTVSSAKTT | TVSIFPPSSEQLTSGGASVVCFLNNFYPKD |
| | APSVYPLAPVCGDTTGSSVTLGCLV | INVKWKIDGSERQNGVLNSWTDQDSKDS |
| | KGYFPEPVTLTWNSGSLSSGVHTFP | TYSMSSTLTLTKDEYERHNSYTCEATHKT |
| | AVLQSDLYTLSSSVTVTSSTWPSQSI | STSPIVKSFNRNEC (SEQ ID NO: 35) |
| | TCNVAHPASSTKVDKKIEPRGPTIKP | |
| | CPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 34) | |
| I23RB168 | QIQLVQSGPELVKPGASVKMSCKTS | DIQMTQSSSSFSVSLGDRVTITCKASEDLY |
| | GYTFTDYYINWVKQRPGQGLEWIG | NRLAWYQQKPGNAPRLLISGATSLGTGV |
| | WIYPAINNTNYNEKFKGKATLTVDT | LSRFSGSGSGKDYTLSITSLQTEDVATYY |
| | SSRTAYMQLNSLTSEDSAVYFCAIS | CQQYWSIPWTFGAGTKLELKRADAAPTV |
| | RKPYFDYWGQGTTLTVSSAKTTAPS | SIFPPSSEQLTSGGASVVCFLNNFYPKDIN |
| | VYPLAPVCGDTTGSSVTLGCLVKG | VKWKIDGSERQNGVLNSWTDQDSKDST |
| | YFPEPVTLTWNSGSLSSGVHTFPAV | YSMSSTLTLTKDEYERHNSYTCEATHKTS |
| | LQSDLYTLSSSVTVTSSTWPSQSITC | TSPIVKSFNRNEC (SEQ ID NO: 37) |
| | NVAHPASSTKVDKKIEPRGPTIKPCP | |
| | PCKCPAPNLLGGPSVFIFPPKIKDVL | |
| | MISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 36) | |
| I23RB169 | QIQLVQSGPELKKPGETVKISCKASG | DIKMTQSPASLAVSLGQRATISCRASKSV |
| | YTFTTVGMQWVQKMPGKGFKWIG | STSDYSYMHWYQQKPGQPPKLLIYLASN |
| | WINTHSGEPKYTDDFKGRFAFSLET | LESGVPARFSGSGSGTDFTLNIHPVEEEDA |
| | SASTAYLQISNLKSEDTATYFCARSS | ATYYCQHSRELPRTFGAGTKLELKRADA |
| | ALTGAFAYWGQGTLVTVSSAKTTA | APTVSIFPPSSEQLTSGGASVVCFLNNFYP |
| | PSVYPLAPVCGDTTGSSVTLGCLVK | KDINVKWKIDGSERQNGVLNSWTDQDSK |
| | GYFPEPVTLTWNSGSLSSGVHTFPA | DSTYSMSSTLTLTKDEYERHNSYTCEATH |
| | VLQSDLYTLSSSVTVTSSTWPSQSIT | KTSTSPIVKSFNRNEC (SEQ ID NO: 39) |
| | CNVAHPASSTKVDKKIEPRGPTIKPC | |
| | PPCKCPAPNLLGGPSVFIFPPKIKDV | |
| | LMISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 38) | |
| I23RB173 | DVQLQESGPGLVKPSQSLSLTCSVT | DIKMTQSPAIMSASPGEKVTITCSASSSVS |
| | GYSITSGYYWNWIRQFPGNKLEWM | YMHWFQQKPGTSPKLWIYSTSNLASGVP |
| | GYISYDGGNNYNPSLKNRISITRDTS | ARFSGSGSGTSYSLTISRMEAEDAATYYC |
| | KNQFFLKLNSVTTEDTATYYCARD | QQRSSYPLTFGDGTKLEMKRADAAPTVSI |
| | AGLLPYYAMDYWGQGTTLTVSSAK | FPPSSEQLTSGGASVVCFLNNFYPKDINV |
| | TTAPSVYPLAPVCGDTTGSSVTLGC | KWKIDGSERQNGVLNSWTDQDSKDSTYS |
| | LVKGYFPEPVTLTWNSGSLSSGVHT | MSSTLTLTKDEYERHNSYTCEATHKTSTS |
| | FPAVLQSDLYTLSSSVTVTSSTWPSQ | PIVKSFNRNEC (SEQ ID NO: 41) |
| | SITCNVAHPASSTKVDKKIEPRGPTI | |
| | KPCPPCKCPAPNLLGGPSVFIFPPKIK | |
| | DVLMISLSPIVTCVVVDVSEDDPDV | |
| | QISWFVNNVEVHTAQTQTHREDYN | |
| | STLRVVSALPIQHQDWMSGKEFKC | |
| | KVNNKDLPAPIERTISKPKGSVRAPQ | |
| | VYVLPPPEEEMTKKQVTLTCMVTD | |
| | FMPEDIYVEWTNNGKTELNYKNTE | |
| | PVLDSDGSYFMYSKLRVEKKNWVE | |
| | RNSYSCSVVHEGLHNHHTTKSFSRT | |
| | PGK (SEQ ID NO: 40) | |
| I23RB177 | EVQLQQSGPELLKPGASVKISCKAS | NTQMTQSPSSMYASLGERVTITCKASQDI |
| | GYTITDYTMHWVKQSHGKSLEWIG | NSYLSWFQQKPGKSPKALIYRANRLVDG |
| | GINPNYGGTSYNEKFKDKATLTVD | VPSRFSGSGSGQDYSLTISSLEYEDMGIYY |
| | KSSSTAYMELRSLTSEDSAVFYCVR | CQQYDEFPRTFGAGTKLELKRADAAPTV |
| | GNIYFDYWGQGTTLTVSSAKTTAPS | SIFPPSSEQLTSGGASVVCFLNNFYPKDIN |
| | VYPLAPVCGDTTGSSVTLGCLVKG | VKWKIDGSERQNGVLNSWTDQDSKDST |
| | YFPEPVTLTWNSGSLSSGVHTFPAV | YSMSSTLTLTKDEYERHNSYTCEATHKTS |
| | LQSDLYTLSSSVTVTSSTWPSQSITC | TSPIVKSFNRNEC (SEQ ID NO: 43) |
| | NVAHPASSTKVDKKIEPRGPTIKPCP | |
| | PCKCPAPNLLGGPSVFIFPPKIKDVL | |
| | MISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 42) | |
| I23RB180 | QVTLKESGPGILQPSQTLSLTCSFSG | DIQMTQSPASLSASVGETVTITCRASVNIH |
| | FSLSTYGMGVGWIRQPSGKGLEWL | NYLAWYQQKQGKSPQLLVYNAKTLADG |
| | ANIWWNDSKYHNAALKSRLTISKD | VPSRFSGSGSGTQYSLKINSLQPEDFGSYY |
| | TSKNQVFLKIASVDTADTATYYCGR | CQHFWTTPYMYTFGAGTKLELKRADAA |
| | IDGYYECVDVWGTGTSVTVSSAKT | PTVSIFPPSSEQLTSGGASVVCFLNNFYPK |
| | TAPSVYPLAPVCGDTTGSSVTLGCL | DINVKWKIDGSERQNGVLNSWTDQDSKD |
| | VKGYFPEPVTLTWNSGSLSSGVHTF | STYSMSSTLTLTKDEYERHNSYTCEATHK |
| | PAVLQSDLYTLSSSVTVTSSTWPSQS | TSTSPIVKSFNRNEC (SEQ ID NO: 45) |
| | ITCNVAHPASSTKVDKKIEPRGPTIK | |
| | PCPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 44) | |
| I23RB182 | EVQLQQSGPGLVKPSQSLSLTCSVT | DIVMTQSPAIMSASLGEKVTMSCRASSSV |
| | GYSITSGYFWNWLRQFPGSKLEWM | NYMYWYQQKSDASPKLWIYYTSNLAPG |
| | GFIDNDGSNDYNPSLKNRISITRDTS | VPARFSGSGSGNSYSLTISSMEGEDAATY |
| | KNQFFLRLNSVTTEDTATYYCARGR | YCQQFTNSPWTFGAGTKLELKRADAAPT |
| | LGFFDSWGPGTTLTVSSAKTTAPSV | VSIFPPSSEQLTSGGASVVCFLNNFYPKDI |
| | YPLAPVCGDTTGSSVTLGCLVKGYF | NVKWKIDGSERQNGVLNSWTDQDSKDS |
| | PEPVTLTWNSGSLSSGVHTFPAVLQ | TYSMSSTLTLTKDEYERHNSYTCEATHKT |
| | SDLYTLSSSVTVTSSTWPSQSITCNV | STSPIVKSFNRNEC (SEQ ID NO: 47) |
| | AHPASSTKVDKKIEPRGPTIKPCPPC | |
| | KCPAPNLLGGPSVFIFPPKIKDVLMI | |
| | SLSPIVTCVVVDVSEDDPDVQISWF | |
| | VNNVEVHTAQTQTHREDYNSTLRV | |
| | VSALPIQHQDWMSGKEFKCKVNNK | |
| | DLPAPIERTISKPKGSVRAPQVYVLP | |
| | PPEEEMTKKQVTLTCMVTDFMPEDI | |
| | YVEWTNNGKTELNYKNTEPVLDSD | |
| | GSYFMYSKLRVEKKNWVERNSYSC | |
| | SVVHEGLHNHHTTKSFSRTPGK | |
| | (SEQ ID NO: 46) | |
| I23RB188 | QIQLVQSGPELKKPGETVKISCKASG | DVQMIQSPSSLSASLGERVSLTCRASQEIS |
| | YTFTNYGMNWVKQAPGKGLKWM | GNLSWLQQKPDGTIKRLIYAASTLDSGVP |
| | GWINTYTGAPTYADDFKGRFAFSLE | KRFSGSRSGSDYSLTISSLESEDFADYYCL |
| | TSASTAYLQINNFKNEDTATYFCAR | QYASYPWTFGAGTRLEIKRADAAPTVSIF |
| | GNGYPYYYGINYWGQGTSVTVSSA | PPSSEQLTSGGASVVCFLNNFYPKDINVK |
| | KTTAPSVYPLAPVCGDTTGSSVTLG | WKIDGSERQNGVLNSWTDQDSKDSTYS |
| | CLVKGYFPEPVTLTWNSGSLSSGVH | MSSTLTLTKDEYERHNSYTCEATHKTSTS |
| | TFPAVLQSDLYTLSSSVTVTSSTWPS | PIVKSFNRNEC (SEQ ID NO: 49) |
| | QSITCNVAHPASSTKVDKKIEPRGPT | |
| | IKPCPPCKCPAPNLLGGPSVFIFPPKI | |
| | KDVLMISLSPIVTCVVVDVSEDDPD | |
| | VQISWFVNNVEVHTAQTQTHREDY | |
| | NSTLRVVSALPIQHQDWMSGKEFK | |
| | CKVNNKDLPAPIERTISKPKGSVRAP | |
| | QVYVLPPPEEEMTKKQVTLTCMVT | |
| | DFMPEDIYVEWTNNGKTELNYKNT | |
| | EPVLDSDGSYFMYSKLRVEKKNWV | |
| | ERNSYSCSVVHEGLHNHHTTKSFSR | |
| | TPGK (SEQ ID NO: 48) | |
| I23RB189 | DVQLQESGPDLVKPSQSLSLTCTVT | DIVLTQSPKSMSMSVGERVTLSCKASENV |
| | GYSITSGYNWHWIRQFPGNKLEWM | GTYVSWYQQKPEQSPKLLMYGASNRYT |
| | GYIHSSGTTNYSPSLRSRLSITRDTSK | GVPDRFTGSGSATDFTLTISSVQAEDLAD |
| | NQFFLQLNSVTTEDTATYYCTRQLG | YHCGQSYSYPPTFGGGTKLEIKRADAAPT |
| | LKAMEYWGQGTTLTVSSAKTTAPS | VSIFPPSSEQLTSGGASVVCFLNNFYPKDI |
| | VYPLAPVCGDTTGSSVTLGCLVKG | NVKWKIDGSERQNGVLNSWTDQDSKDS |
| | YFPEPVTLTWNSGSLSSGVHTFPAV | TYSMSSTLTLTKDEYERHNSYTCEATHKT |
| | LQSDLYTLSSSVTVTSSTWPSQSITC | STSPIVKSFNRNEC (SEQ ID NO: 51) |
| | NVAHPASSTKVDKKIEPRGPTIKPCP | |
| | PCKCPAPNLLGGPSVFIFPPKIKDVL | |
| | MISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 50) | |
| I23RB27 | QVQLKESGPDLVQPSQTLSLTCTVS | DIQMTQSPTSLAVSLGQRATISCRASESVE |
| | GFSLTSYGVHWVRQPPGKGLEWVG | YYGTSLMHWYQQKPGQPPKLLIYAASNV |
| | TLGWDDKKSYNSALKSRLSISRDTS | ESGVPARFSGSGSGTDFSLNIHPVEEEDIA |
| | KSQVFLKLSSLQTEDTAMYYCTKE | MYFCQQGKKVPWTFGAGTKLEMKRADA |
| | DVSYAMDFWGQGTTLTVSSAKTTA | APTVSIFPPSSEQLTSGGASVVCFLNNFYP |
| | PSVYPLAPVCGDTTGSSVTLGCLVK | KDINVKWKIDGSERQNGVLNSWTDQDSK |
| | GYFPEPVTLTWNSGSLSSGVHTFPA | DSTYSMSSTLTLTKDEYERHNSYTCEATH |
| | VLQSDLYTLSSSVTVTSSTWPSQSIT | KTSTSPIVKSFNRNEC (SEQ ID NO: 53) |
| | CNVAHPASSTKVDKKIEPRGPTIKPC | |
| | PPCKCPAPNLLGGPSVFIFPPKIKDV | |
| | LMISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 52) | |
| I23RB30 | QVTLKESGPGILQPSQTLSLTCSFSG | DIVLTQSQRFMSTSVGDRVSITCKASQNIR |
| | FSLSTSGMGVSWIRQPSGKGLEWLA | TAVAWYQQKPGQSPKALIYLPSNRHTGV |
| | HIYWDDDKRYNPSLKSRLTISKDTS | PDRFTGSGSGTDFTLTISNVQSEDLADYF |
| | SNQIFLKITSVDTADTATYYCARRTL | CLQHWNYPLTFGAGTKLELKRADAAPTV |
| | GGPPFDYWGQGTTLTVSSAKTTAPS | SIFPPSSEQLTSGGASVVCFLNNFYPKDIN |
| | VYPLAPVCGDTTGSSVTLGCLVKG | VKWKIDGSERQNGVLNSWTDQDSKDST |
| | YFPEPVTLTWNSGSLSSGVHTFPAV | YSMSSTLTLTKDEYERHNSYTCEATHKTS |
| | LQSDLYTLSSSVTVTSSTWPSQSITC | TSPIVKSFNRNEC (SEQ ID NO: 55) |
| | NVAHPASSTKVDKKIEPRGPTIKPCP | |
| | PCKCPAPNLLGGPSVFIFPPKIKDVL | |
| | MISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 54) | |
| I23RB31 | EFQLQQSGAEMVRPGASVKLSCKA | DIVLTQSPATLSVTPGDRVSLSCRASQSIS |
| | SGYTFTGYWMNWVKQRPGQGLEW | DHLHWYQQKSHESPRLLIKFASQSISGIPS |
| | IGKIDPSDTETHYNQNFKDKATLTV | RFSGSGSGSDFTLSIDSVEPEDVGVYYCQ |
| | DKSSSTAYMQLNSLTSEDSAVYYC | NGHSFPLTFGSGTKLEIKRADAAPTVSIFP |
| | ARWGLGRNFAMDYWGQGTTLTVS | PSSEQLTSGGASVVCFLNNFYPKDINVKW |
| | SAKTTAPSVYPLAPVCGDTTGSSVT | KIDGSERQNGVLNSWTDQDSKDSTYSMS |
| | LGCLVKGYFPEPVTLTWNSGSLSSG | STLTLTKDEYERHNSYTCEATHKTSTSPIV |
| | VHTFPAVLQSDLYTLSSSVTVTSST | KSFNRNEC (SEQ ID NO: 57) |
| | WPSQSITCNVAHPASSTKVDKKIEP | |
| | RGPTIKPCPPCKCPAPNLLGGPSVFIF | |
| | PPKIKDVLMISLSPIVTCVVVDVSED | |
| | DPDVQISWFVNNVEVHTAQTQTHR | |
| | EDYNSTLRVVSALPIQHQDWMSGK | |
| | EFKCKVNNKDLPAPIERTISKPKGSV | |
| | RAPQVYVLPPPEEEMTKKQVTLTC | |
| | MVTDFMPEDIYVEWTNNGKTELNY | |
| | KNTEPVLDSDGSYFMYSKLRVEKK | |
| | NWVERNSYSCSVVHEGLHNHHTTK | |
| | SFSRTPGK (SEQ ID NO: 56) | |
| I23RB32 | EFQLQQSGPELVKPGASVKLSCKAS | DIVMTQSPSSLTVTAGEKVTMSCKSSQSL |
| | GYTFTDYALHWVKQSPGQGLEWIG | LNSGNQKNYLTWYQQKPGQPPKLLIYW |
| | WIYPGGGNTYYNDKFKGKATMTA | ASTRESGVPDRFTGSGSGTDFTLTISSVQA |
| | DKSSSTAYMQLSSLTSEDSAVYFCA | EDLAVYYCQNDYTYPLTFGSGTKLEIKR |
| | RGRLRRDYPMDYWGQGTSVTVSSA | ADAAPTVSIFPPSSEQLTSGGASVVCFLNN |
| | KTTAPSVYPLAPVCGDTTGSSVTLG | FYPKDINVKWKIDGSERQNGVLNSWTDQ |
| | CLVKGYFPEPVTLTWNSGSLSSGVH | DSKDSTYSMSSTLTLTKDEYERHNSYTCE |
| | TFPAVLQSDLYTLSSSVTVTSSTWPS | ATHKTSTSPIVKSFNRNEC (SEQ ID NO: 59) |
| | QSITCNVAHPASSTKVDKKIEPRGPT | |
| | IKPCPPCKCPAPNLLGGPSVFIFPPKI | |
| | KDVLMISLSPIVTCVVVDVSEDDPD | |
| | VQISWFVNNVEVHTAQTQTHREDY | |
| | NSTLRVVSALPIQHQDWMSGKEFK | |
| | CKVNNKDLPAPIERTISKPKGSVRAP | |
| | QVYVLPPPEEEMTKKQVTLTCMVT | |
| | DFMPEDIYVEWTNNGKTELNYKNT | |
| | EPVLDSDGSYFMYSKLRVEKKNWV | |
| | ERNSYSCSVVHEGLHNHHTTKSFSR | |
| | TPGK (SEQ ID NO: 58) | |
| I23RB33 | EVQLQQSGAELVKPGASVKLSCTAS | DIVLTQSPAIMSASPGEKVTMTCRASSSV |
| | GFNIRDIYIHWVKQRPEQGLEWIGRI | RSSYLHWYQQKSGASPKLWIYSTSNLAS |
| | DPANGHSIYAAKFQGKATITADTSL | GVPARFSGSGSGTSYSLTITSVEAEDAAT |
| | NIAYMQLSSLTSGDTAVYYCARGS | YYCQQYTGNPIFTFGSGTKLELKRADAAP |
| | DNDYGRVLAYWGLGTSVTVSSAKT | TVSIFPPSSEQLTSGGASVVCFLNNFYPKD |
| | TAPSVYPLAPVCGDTTGSSVTLGCL | INVKWKIDGSERQNGVLNSWTDQDSKDS |
| | VKGYFPEPVTLTWNSGSLSSGVHTF | TYSMSSTLTLTKDEYERHNSYTCEATHKT |
| | PAVLQSDLYTLSSSVTVTSSTWPSQS | STSPIVKSFNRNEC (SEQ ID NO: 61) |
| | ITCNVAHPASSTKVDKKIEPRGPTIK | |
| | PCPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 60) | |
| I23RB35 | DVQLQESGPGLVKPSQSLSLTCSVT | DIVLTQSPASLAVSLGQRATISCRASESVD |
| | GYSITSGHNWNWIRQFPGNKLEWL | SYGNSFMHWYQQKPGQPPKLLIYRASNL |
| | GYISYDGSNNYDPSLKNRISITRDTS | ESEIPARFSGRGSGTDFTLTINPVEADDVA |
| | KNQFFLKLNSVTTEDTATYYCARG | TYYCQQSNADPYTFGGGTKLEIKRADAA |
| | GGYFGNSYRVFASWGQGTTVTVSS | PTVSIFPPSSEQLTSGGASVVCFLNNFYPK |
| | AKTTAPSVYPLAPVCGDTTGSSVTL | DINVKWKIDGSERQNGVLNSWTDQDSKD |
| | GCLVKGYFPEPVTLTWNSGSLSSGV | STYSMSSTLTLTKDEYERHNSYTCEATHK |
| | HTFPAVLQSDLYTLSSSVTVTSSTW | TSTSPIVKSFNRNEC (SEQ ID NO: 63) |
| | PSQSITCNVAHPASSTKVDKKIEPRG | |
| | PTIKPCPPCKCPAPNLLGGPSVFIFPP | |
| | KIKDVLMISLSPIVTCVVVDVSEDDP | |
| | DVQISWFVNNVEVHTAQTQTHRED | |
| | YNSTLRVVSALPIQHQDWMSGKEF | |
| | KCKVNNKDLPAPIERTISKPKGSVR | |
| | APQVYVLPPPEEEMTKKQVTLTCM | |
| | VTDFMPEDIYVEWTNNGKTELNYK | |
| | NTEPVLDSDGSYFMYSKLRVEKKN | |
| | WVERNSYSCSVVHEGLHNHHTTKS | |
| | FSRTPGK (SEQ ID NO: 62) | |
| I23RB36 | QIQLVQSGAELVKPGASVKLSCTTS | DIVMTQSPKSMSMSVGERVTLSCKASEN |
| | GFNIKDTYIHWMKQRPEQGLEWIG | VGTSVSWFQQKPEQSPKLLIYGASNRYTG |
| | RIDPANGNTIYASKFQGKATLTTDT | VPDRFTGSGSATDFTLTISSVQAEDLADY |
| | SSNTAYMLLSSLTSGDTAVYYCTRG | HCGQSYSYPRTFGGGTKLEIKRADAAPTV |
| | TSWFAYWGQGTTVTVSSAKTTAPS | SIFPPSSEQLTSGGASVVCFLNNFYPKDIN |
| | VYPLAPVCGDTTGSSVTLGCLVKG | VKWKIDGSERQNGVLNSWTDQDSKDST |
| | YFPEPVTLTWNSGSLSSGVHTFPAV | YSMSSTLTLTKDEYERHNSYTCEATHKTS |
| | LQSDLYTLSSSVTVTSSTWPSQSITC | TSPIVKSFNRNEC (SEQ ID NO: 65) |
| | NVAHPASSTKVDKKIEPRGPTIKPCP | |
| | PCKCPAPNLLGGPSVFIFPPKIKDVL | |
| | MISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 64) | |
| I23RB39 | EVQLQQSGPELVKPGAPVKISCKAS | DIQMTQSHKFMSTSVGDRVSITCKASQD |
| | GYTFTNYYIHWVKQRPGQGLEWIG | VSTAVAWYQQKPGQSPKLLIYSAFYRYT |
| | YIYPGNGGTAYNQKFKDKATLTAD | GVPDRFTGSGSGTDFTFTISSVQAEDLAV |
| | NPSSTAYMQLSSLTSEDSAVYFCAR | YYCQQHYSSPRTFGAGTKLELKRADAAP |
| | SRGGSRNYFDYWGQGTTLTVSSAK | TVSIFPPSSEQLTSGGASVVCFLNNFYPKD |
| | TTAPSVYPLAPVCGDTTGSSVTLGC | INVKWKIDGSERQNGVLNSWTDQDSKDS |
| | LVKGYFPEPVTLTWNSGSLSSGVHT | TYSMSSTLTLTKDEYERHNSYTCEATHKT |
| | FPAVLQSDLYTLSSSVTVTSSTWPSQ | STSPIVKSFNRNEC (SEQ ID NO: 67) |
| | SITCNVAHPASSTKVDKKIEPRGPTI | |
| | KPCPPCKCPAPNLLGGPSVFIFPPKIK | |
| | DVLMISLSPIVTCVVVDVSEDDPDV | |
| | QISWFVNNVEVHTAQTQTHREDYN | |
| | STLRVVSALPIQHQDWMSGKEFKC | |
| | KVNNKDLPAPIERTISKPKGSVRAPQ | |
| | VYVLPPPEEEMTKKQVTLTCMVTD | |
| | FMPEDIYVEWTNNGKTELNYKNTE | |
| | PVLDSDGSYFMYSKLRVEKKNWVE | |
| | RNSYSCSVVHEGLHNHHTTKSFSRT | |
| | PGK (SEQ ID NO: 66) | |
| I23RB42 | EVQLQQSGAELVKPGASVKISCKAS | DIVLTQTAPSVPVTPGESVSISCRSSKSLL |
| | GYSFTDYIMNWVKQSHGKSLEWIG | HSNGNTYLYWFLQRPGQSPQLLIYRMSN |
| | NINPYFGFTSYNQEFKGKATLTVDK | LASGVPDRESGSGSGTAFTLRISRVEAED |
| | SSSTAYMLLNSLTSEDSAVYYCSRG | VGVYYCMQHLEYPYTFGSGTRLEIKRAD |
| | SYYGYETFFDYWGQGTTLTVSSAK | AAPTVSIFPPSSEQLTSGGASVVCFLNNFY |
| | TTAPSVYPLAPVCGDTTGSSVTLGC | PKDINVKWKIDGSERQNGVLNSWTDQDS |
| | LVKGYFPEPVTLTWNSGSLSSGVHT | KDSTYSMSSTLTLTKDEYERHNSYTCEAT |
| | FPAVLQSDLYTLSSSVTVTSSTWPSQ | HKTSTSPIVKSFNRNEC (SEQ ID NO: 69) |
| | SITCNVAHPASSTKVDKKIEPRGPTI | |
| | KPCPPCKCPAPNLLGGPSVFIFPPKIK | |
| | DVLMISLSPIVTCVVVDVSEDDPDV | |
| | QISWFVNNVEVHTAQTQTHREDYN | |
| | STLRVVSALPIQHQDWMSGKEFKC | |
| | KVNNKDLPAPIERTISKPKGSVRAPQ | |
| | VYVLPPPEEEMTKKQVTLTCMVTD | |
| | FMPEDIYVEWTNNGKTELNYKNTE | |
| | PVLDSDGSYFMYSKLRVEKKNWVE | |
| | RNSYSCSVVHEGLHNHHTTKSFSRT | |
| | PGK (SEQ ID NO: 68) | |
| I23RB45 | QVTLKESGPGILQPSQTLSLTCSFSG | DIVLTQSQKFISTSVGDRVSITCKASQNVR |
| | FSLSTYGMGVSWIRQPSGKGLEWL | TAVAWYQQKPGQSPKLLIYLPSNRHTGV |
| | AHIYWDDDKRYNPSLKSRLTISKDT | PDRFTGSGSGTVFTLTISNVQSEDQADYF |
| | SNNQVFLKITSVDTADTATYYCAQR | CLQHWNYPLTFGAGTKLELKRADAAPTV |
| | ALPLLGQKAPFDYWGQGTTLTVSS | SIFPPSSEQLTSGGASVVCFLNNFYPKDIN |
| | AKTTAPSVYPLAPVCGDTTGSSVTL | VKWKIDGSERQNGVLNSWTDQDSKDST |
| | GCLVKGYFPEPVTLTWNSGSLSSGV | YSMSSTLTLTKDEYERHNSYTCEATHKTS |
| | HTFPAVLQSDLYTLSSSVTVTSSTW | TSPIVKSFNRNEC (SEQ ID NO: 71) |
| | PSQSITCNVAHPASSTKVDKKIEPRG | |
| | PTIKPCPPCKCPAPNLLGGPSVFIFPP | |
| | KIKDVLMISLSPIVTCVVVDVSEDDP | |
| | DVQISWFVNNVEVHTAQTQTHRED | |
| | YNSTLRVVSALPIQHQDWMSGKEF | |
| | KCKVNNKDLPAPIERTISKPKGSVR | |
| | APQVYVLPPPEEEMTKKQVTLTCM | |
| | VTDFMPEDIYVEWTNNGKTELNYK | |
| | NTEPVLDSDGSYFMYSKLRVEKKN | |
| | WVERNSYSCSVVHEGLHNHHTTKS | |
| | FSRTPGK (SEQ ID NO: 70) | |
| I23RB47 | QVQLKESGPGLVAPSQSLSITCTVSG | DIVLTQSPASQSASLGESVTITCLASQTIGS |
| | FSLTNYDIHWVRQSPGKGLEWLGVI | WLAWYQQKPGKSPQLLIYAATRLADGVP |
| | WSDGRTDYKGAFISRLSISKDNSKS | SRFSGSGSGTKFSFKISSLQAEDFVFYYCH |
| | QVFFKMNSLQADDTAIYYCARNNY | QLYSIPLTFGGGTKLEMKRADAAPTVSIF |
| | YRYGRNTMDYWGQGTSVTVSSAK | PPSSEQLTSGGASVVCFLNNFYPKDINVK |
| | TTAPSVYPLAPVCGDTTGSSVTLGC | WKIDGSERQNGVLNSWTDQDSKDSTYS |
| | LVKGYFPEPVTLTWNSGSLSSGVHT | MSSTLTLTKDEYERHNSYTCEATHKTSTS |
| | FPAVLQSDLYTLSSSVTVTSSTWPSQ | PIVKSFNRNEC (SEQ ID NO: 73) |
| | SITCNVAHPASSTKVDKKIEPRGPTI | |
| | KPCPPCKCPAPNLLGGPSVFIFPPKIK | |
| | DVLMISLSPIVTCVVVDVSEDDPDV | |
| | QISWFVNNVEVHTAQTQTHREDYN | |
| | STLRVVSALPIQHQDWMSGKEFKC | |
| | KVNNKDLPAPIERTISKPKGSVRAPQ | |
| | VYVLPPPEEEMTKKQVTLTCMVTD | |
| | FMPEDIYVEWTNNGKTELNYKNTE | |
| | PVLDSDGSYFMYSKLRVEKKNWVE | |
| | RNSYSCSVVHEGLHNHHTTKSFSRT | |
| | PGK (SEQ ID NO: 72) | |
| I23RB51 | QVQLQQSGPDLVKPSQSLSLTCTVT | DIVLTQSPASLSASVGETVTITCRASGNIH |
| | GYSITSGYNWHWIRQFPGNKLEWM | NYLAWYQHKQGKSPQLLVYNAKTLADG |
| | GYVHYRDSTHYNPSLKSRISITRDTS | VPSRFSGSGSGTQYSLKINSLQPEDFGSYY |
| | KNQFFLQLNSVTTEDTATYYCARQL | CQYFWTTRTFGAGTKLELKRADAAPTVS |
| | GLKAMDYWGQGTSVTVSSAKTTAP | IFPPSSEQLTSGGASVVCFLNNFYPKDINV |
| | SVYPLAPVCGDTTGSSVTLGCLVKG | KWKIDGSERQNGVLNSWTDQDSKDSTYS |
| | YFPEPVTLTWNSGSLSSGVHTFPAV | MSSTLTLTKDEYERHNSYTCEATHKTSTS |
| | LQSDLYTLSSSVTVTSSTWPSQSITC | PIVKSFNRNEC (SEQ ID NO: 75) |
| | NVAHPASSTKVDKKIEPRGPTIKPCP | |
| | PCKCPAPNLLGGPSVFIFPPKIKDVL | |
| | MISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 74) | |
| I23RB54 | EVQLQQSGGGLVKPGGSLKLSCAA | DIQMTQSPKFLLVSAGDRVTITCKASQSV |
| | SGFTFSSYAMSWVRQTPEKRLEWV | SNDVAWYQQKPGQSPRLLIYYASNRYTG |
| | AGISSGGSYIYYPDNVKGRFTISRDN | VPDRFTGSGYGTDFTFTISTVQAEDLAVY |
| | ARNTLYLQMSSLRSEDTAMYYCAR | FCQQDYSSLPTFGAGTKLEMKRADAAPT |
| | KKSDYGAMDYWGQGTTLTVSSAK | VSIFPPSSEQLTSGGASVVCFLNNFYPKDI |
| | TTAPSVYPLAPVCGDTTGSSVTLGC | NVKWKIDGSERQNGVLNSWTDQDSKDS |
| | LVKGYFPEPVTLTWNSGSLSSGVHT | TYSMSSTLTLTKDEYERHNSYTCEATHKT |
| | FPAVLQSDLYTLSSSVTVTSSTWPSQ | STSPIVKSFNRNEC (SEQ ID NO: 77) |
| | SITCNVAHPASSTKVDKKIEPRGPTI | |
| | KPCPPCKCPAPNLLGGPSVFIFPPKIK | |
| | DVLMISLSPIVTCVVVDVSEDDPDV | |
| | QISWFVNNVEVHTAQTQTHREDYN | |
| | STLRVVSALPIQHQDWMSGKEFKC | |
| | KVNNKDLPAPIERTISKPKGSVRAPQ | |
| | VYVLPPPEEEMTKKQVTLTCMVTD | |
| | FMPEDIYVEWTNNGKTELNYKNTE | |
| | PVLDSDGSYFMYSKLRVEKKNWVE | |
| | RNSYSCSVVHEGLHNHHTTKSFSRT | |
| | PGK (SEQ ID NO: 76) | |
| I23RB56 | EVKLVESGGGLVKPGGSLKLSCAAS | DIVMTQSPKFLLVSAGDRVTITCKASQSV |
| | GFTFSSYAMSWVRQTPEKRLEWVA | SNDVAWYQQKPGQSPKLLIYYASNRYTG |
| | AINSNDGSTYYPDTVKDRFTISRDN | VPDRFTGSGYGTVFTFTISTVQAEDLAVY |
| | AKNTLYLQMSSLRSEDTALYYCAR | FCQQDYSSPFTFGSGTKLEIKRADAAPTV |
| | HFLYFSNFYYFDYWGQGTTLTVSSA | SIFPPSSEQLTSGGASVVCFLNNFYPKDIN |
| | KTTAPSVYPLAPVCGDTTGSSVTLG | VKWKIDGSERQNGVLNSWTDQDSKDST |
| | CLVKGYFPEPVTLTWNSGSLSSGVH | YSMSSTLTLTKDEYERHNSYTCEATHKTS |
| | TFPAVLQSDLYTLSSSVTVTSSTWPS | TSPIVKSFNRNEC (SEQ ID NO: 79) |
| | QSITCNVAHPASSTKVDKKIEPRGPT | |
| | IKPCPPCKCPAPNLLGGPSVFIFPPKI | |
| | KDVLMISLSPIVTCVVVDVSEDDPD | |
| | VQISWFVNNVEVHTAQTQTHREDY | |
| | NSTLRVVSALPIQHQDWMSGKEFK | |
| | CKVNNKDLPAPIERTISKPKGSVRAP | |
| | QVYVLPPPEEEMTKKQVTLTCMVT | |
| | DFMPEDIYVEWTNNGKTELNYKNT | |
| | EPVLDSDGSYFMYSKLRVEKKNWV | |
| | ERNSYSCSVVHEGLHNHHTTKSFSR | |
| | TPGK (SEQ ID NO: 78) | |
| I23RB57 | EFQLQQSGPELVKPGASVKMSCKAS | DIVMTQSPAIMSASLGERVTMTCTASSSV |
| | GYTFTNYYMHWVKQSHGKTIEWIG | SSSYLHWYQQKPGSSPKLWIYSTSNLASG |
| | YIYPNSGDTSYNQKFKGKATLTVDK | VPARFSGSGSGTSYSLTISSMEAEDAATY |
| | SSSTAYMELRSLTSEDSAVYNCARF | YCHQYHRSPFTFGSGTKLEIKRADAAPTV |
| | GGFYDAMDYWGQGTSVTVSSAKT | SIFPPSSEQLTSGGASVVCFLNNFYPKDIN |
| | TAPSVYPLAPVCGDTTGSSVTLGCL | VKWKIDGSERQNGVLNSWTDQDSKDST |
| | VKGYFPEPVTLTWNSGSLSSGVHTF | YSMSSTLTLTKDEYERHNSYTCEATHKTS |
| | PAVLQSDLYTLSSSVTVTSSTWPSQS | TSPIVKSFNRNEC (SEQ ID NO: 81) |
| | ITCNVAHPASSTKVDKKIEPRGPTIK | |
| | PCPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 80) | |
| I23RB58 | EVKLVESGGGLVKPGGSLKLSCAAS | DIVMTQTPASLAVSLGQRATISCRASESV |
| | GFSFIIYTMSWVRQTPEKRLEWVAY | DSYGNSFMHWYQQKPGQPPKLLIYRASN |
| | ITSGGGITYYPDSVKGRFTISRDNAR | LESGIPARFSGSGSRTDFTLTINPVEADDV |
| | NTLYLQMSSLRSEDTAMYYCARHE | ATYYCQQSNEDPLTFGAGTKLEMKRADA |
| | GDYYDDYYRIGYAMDYWGQGTSV | APTVSIFPPSSEQLTSGGASVVCFLNNFYP |
| | TVSSAKTTAPSVYPLAPVCGDTTGS | KDINVKWKIDGSERQNGVLNSWTDQDSK |
| | SVTLGCLVKGYFPEPVTLTWNSGSL | DSTYSMSSTLTLTKDEYERHNSYTCEATH |
| | SSGVHTFPAVLQSDLYTLSSSVTVTS | KTSTSPIVKSFNRNEC (SEQ ID NO: 83) |
| | STWPSQSITCNVAHPASSTKVDKKIE | |
| | PRGPTIKPCPPCKCPAPNLLGGPSVFI | |
| | FPPKIKDVLMISLSPIVTCVVVDVSE | |
| | DDPDVQISWFVNNVEVHTAQTQTH | |
| | REDYNSTLRVVSALPIQHQDWMSG | |
| | KEFKCKVNNKDLPAPIERTISKPKGS | |
| | VRAPQVYVLPPPEEEMTKKQVTLTC | |
| | MVTDFMPEDIYVEWTNNGKTELNY | |
| | KNTEPVLDSDGSYFMYSKLRVEKK | |
| | NWVERNSYSCSVVHEGLHNHHTTK | |
| | SFSRTPGK (SEQ ID NO: 82) | |
| I23RB60 | DVQLQESGPELVKPGDSVKMSCEA | DIVMTQSTSSLSASLGDRVTISCRASQDIR |
| | SGYTFTDYYIDWVKQSHGKSLEWI | NNLNWYQQKPDGTVKLLIFYSSRLHSGV |
| | GYIYPNNGGISFNQKFEGKATLTVD | PSRFSGSGSGTDYSLTISNLEQEDIATYFC |
| | KSSSTAYMELHSLTSEDSAVYYCAR | QQGHTLPWTFGAGTKLELKRADAAPTVS |
| | EKLRGRDYWGQGTTLTVSSAKTTA | IFPPSSEQLTSGGASVVCFLNNFYPKDINV |
| | PSVYPLAPVCGDTTGSSVTLGCLVK | KWKIDGSERQNGVLNSWTDQDSKDSTYS |
| | GYFPEPVTLTWNSGSLSSGVHTFPA | MSSTLTLTKDEYERHNSYTCEATHKTSTS |
| | VLQSDLYTLSSSVTVTSSTWPSQSIT | PIVKSFNRNEC (SEQ ID NO: 85) |
| | CNVAHPASSTKVDKKIEPRGPTIKPC | |
| | PPCKCPAPNLLGGPSVFIFPPKIKDV | |
| | LMISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 84) | |
| I23RB61 | DVQLQESGAELVKPGASVKLSCKSF | DIQMTQSHKFLSTSVGDRITITCKASQDVI |
| | DYTFTSFDINWMKQRPGQGLEWIG | TAVAWYQQKSGQSPKLLIYWASTRHTGV |
| | WIFPGSGTTNYNEKFKGKATLTADK | PDRFTGSGSGTDYTLTISNVQAEDLALYY |
| | SSSTAYMHLTSLTSEDSAVYFCARR | CQQHYTTPPTFGAGTKLELKRADAAPTV |
| | LTGTRYFDVWGTGTTLTVSSAKTT | SIFPPSSEQLTSGGASVVCFLNNFYPKDIN |
| | APSVYPLAPVCGDTTGSSVTLGCLV | VKWKIDGSERQNGVLNSWTDQDSKDST |
| | KGYFPEPVTLTWNSGSLSSGVHTFP | YSMSSTLTLTKDEYERHNSYTCEATHKTS |
| | AVLQSDLYTLSSSVTVTSSTWPSQSI | TSPIVKSFNRNEC (SEQ ID NO: 87) |
| | TCNVAHPASSTKVDKKIEPRGPTIKP | |
| | CPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 86) | |
| I23RB65 | EVQLQQSGPELVKPGASVKISCKAS | QIVLTQSPASLAVSLGQRATISCRASQSVS |
| | GYTFTDHTMDWVKQSHGKSLEWIG | TSSYNYIHWYQQKPGQPPKLLIKYASNLE |
| | YIYPKSGNAAYSQKFKSEATLTVDK | SGVPARFSGSGSGTDFTLNIHPVEEDDTA |
| | SSSTAYMEFHSLTSEDSAVYYCAR | TYYCQHTWEIPYTFGSGTKLEIKRADAAP |
| | WGLRRDGFTYWGQGTSVTVSSAKT | TVSIFPPSSEQLTSGGASVVCFLNNFYPKD |
| | TAPSVYPLAPVCGDTTGSSVTLGCL | INVKWKIDGSERQNGVLNSWTDQDSKDS |
| | VKGYFPEPVTLTWNSGSLSSGVHTF | TYSMSSTLTLTKDEYERHNSYTCEATHKT |
| | PAVLQSDLYTLSSSVTVTSSTWPSQS | STSPIVKSFNRNEC (SEQ ID NO: 89) |
| | ITCNVAHPASSTKVDKKIEPRGPTIK | |
| | PCPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 88) | |
| I23RB66 | QVTLKESGPGILQPSQTLSLTCSFSG | DIVMTQSHKFMSTSVGDRVSITCKASQD |
| | FSLSTSNMGIGWIRQPSGKGLEWLA | VSTAVAWYQQKPGQSPKLLIYSASYRYT |
| | HIWWNSDKYYNPSLKSRLTISKDTS | GVPDRFTGSGSGTDFTFTFSSVQAEDLAV |
| | NNQVFLTITSVDTADTATYYCAQIR | YYCQQHYSTPYTFGGGTKLEIKRADAAP |
| | WLRFYFDYWGQGTTVTVSSAKTTA | TVSIFPPSSEQLTSGGASVVCFLNNFYPKD |
| | PSVYPLAPVCGDTTGSSVTLGCLVK | INVKWKIDGSERQNGVLNSWTDQDSKDS |
| | GYFPEPVTLTWNSGSLSSGVHTFPA | TYSMSSTLTLTKDEYERHNSYTCEATHKT |
| | VLQSDLYTLSSSVTVTSSTWPSQSIT | STSPIVKSFNRNEC (SEQ ID NO: 91) |
| | CNVAHPASSTKVDKKIEPRGPTIKPC | |
| | PPCKCPAPNLLGGPSVFIFPPKIKDV | |
| | LMISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 90) | |
| I23RB67 | EVQLQQSGAELVKPGASVKLSCKA | DVVMTQSHKFMSTSVGDRVSITCKASQD |
| | SGYTFTSYWINWVKQRPGQGLEWI | VSTAVAWYQQKPGQSPKLLIYSAFYRYT |
| | GNIYPGSSSTNYNEKFKSKATLTVD | GVPDRFTGSGSGTDFTFTISSVQAEDLAV |
| | TSSSTAYMQLSSLTSDDSAVYYCAR | YYCQQHYSTPYTFGGGTKLELKRADAAP |
| | SAYGNRLAMDYWGQGTTLTVSSA | TVSIFPPSSEQLTSGGASVVCFLNNFYPKD |
| | KTTAPSVYPLAPVCGDTTGSSVTLG | INVKWKIDGSERQNGVLNSWTDQDSKDS |
| | CLVKGYFPEPVTLTWNSGSLSSGVH | TYSMSSTLTLTKDEYERHNSYTCEATHKT |
| | TFPAVLQSDLYTLSSSVTVTSSTWPS | STSPIVKSFNRNEC (SEQ ID NO: 93) |
| | QSITCNVAHPASSTKVDKKIEPRGPT | |
| | IKPCPPCKCPAPNLLGGPSVFIFPPKI | |
| | KDVLMISLSPIVTCVVVDVSEDDPD | |
| | VQISWFVNNVEVHTAQTQTHREDY | |
| | NSTLRVVSALPIQHQDWMSGKEFK | |
| | CKVNNKDLPAPIERTISKPKGSVRAP | |
| | QVYVLPPPEEEMTKKQVTLTCMVT | |
| | DFMPEDIYVEWTNNGKTELNYKNT | |
| | EPVLDSDGSYFMYSKLRVEKKNWV | |
| | ERNSYSCSVVHEGLHNHHTTKSFSR | |
| | TPGK (SEQ ID NO: 92) | |
| I23RB69 | EVQLQQSGAELVKPGASVKISCKAS | DIVMTQSHKFMSTSVGDRVSITCKASQD |
| | GYSFTGYNMNWVKQSHGKSLEWIG | VSTAVAWYQQKPGQSPKLLIYSASYRYT |
| | NINPFYDKTSYNQKFKGKATLTVDK | GVPDRFTGSGSGTDFTFTISSVQAEDLAV |
| | ASSTAYMQLNSLTSEDSAVYYCAR | YYCQQHYSTPWTFGGGTKLEMKRADAA |
| | QLGLKGFAYWGQGTLVTVSAAKTT | PTVSIFPPSSEQLTSGGASVVCFLNNFYPK |
| | APSVYPLAPVCGDTTGSSVTLGCLV | DINVKWKIDGSERQNGVLNSWTDQDSKD |
| | KGYFPEPVTLTWNSGSLSSGVHTFP | STYSMSSTLTLTKDEYERHNSYTCEATHK |
| | AVLQSDLYTLSSSVTVTSSTWPSQSI | TSTSPIVKSFNRNEC (SEQ ID NO: 95) |
| | TCNVAHPASSTKVDKKIEPRGPTIKP | |
| | CPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 94) | |
| I23RB71 | DVQLQESGPDLVKPSQSLSLTCTVT | DIVMTQSHKFMSTSVGDRVSITCKASQD |
| | DYSITRGYNWHWIRQFPGNKLEWM | VITAVAWYQQKPGQSPKLLIYSASYRYT |
| | GYIHYSGSTNYNPSLKSRISITRDTS | GVPDRFTGSGSGTDFTFTISSVQAEDLAV |
| | KNQFFLQLNSVTTEDTATYYCARQ | YFCQQHFGTPYTFGSGTKLEMKRADAAP |
| | YGLKAMDYWGQGTSVTVSSAKTT | TVSIFPPSSEQLTSGGASVVCFLNNFYPKD |
| | APSVYPLAPVCGDTTGSSVTLGCLV | INVKWKIDGSERQNGVLNSWTDQDSKDS |
| | KGYFPEPVTLTWNSGSLSSGVHTFP | TYSMSSTLTLTKDEYERHNSYTCEATHKT |
| | AVLQSDLYTLSSSVTVTSSTWPSQSI | STSPIVKSFNRNEC (SEQ ID NO: 97) |
| | TCNVAHPASSTKVDKKIEPRGPTIKP | |
| | CPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 96) | |
| I23RB73 | EVQLQQSGAELVKPGASVKISCKAS | DIVMTQSHKFMSTSVGDRVSITCKASQD |
| | GYTFTDHAIHWVKQRPEQGLEWIG | VSTAVAWYQQKPGQSPKLLIYWASTRLT |
| | YISLGNGDIKYSEKFRGKATLTADK | GVPDRFTGSGSGTDYTLTISSVQAEDLAL |
| | TSNTAYMQLNSLTSEDSAVYFCKRS | YYCQQYYNTPLTFGAGTKLELKRADAAP |
| | RVLRWTTYWYFDVWGAGTSVTVS | TVSIFPPSSEQLTSGGASVVCFLNNFYPKD |
| | SAKTTAPSVYPLAPVCGDTTGSSVT | INVKWKIDGSERQNGVLNSWTDQDSKDS |
| | LGCLVKGYFPEPVTLTWNSGSLSSG | TYSMSSTLTLTKDEYERHNSYTCEATHKT |
| | VHTFPAVLQSDLYTLSSSVTVTSST | STSPIVKSFNRNEC (SEQ ID NO: 99) |
| | WPSQSITCNVAHPASSTKVDKKIEP | |
| | RGPTIKPCPPCKCPAPNLLGGPSVFIF | |
| | PPKIKDVLMISLSPIVTCVVVDVSED | |
| | DPDVQISWFVNNVEVHTAQTQTHR | |
| | EDYNSTLRVVSALPIQHQDWMSGK | |
| | EFKCKVNNKDLPAPIERTISKPKGSV | |
| | RAPQVYVLPPPEEEMTKKQVTLTC | |
| | MVTDFMPEDIYVEWTNNGKTELNY | |
| | KNTEPVLDSDGSYFMYSKLRVEKK | |
| | NWVERNSYSCSVVHEGLHNHHTTK | |
| | SFSRTPGK (SEQ ID NO: 98) | |
| I23RB74 | DVQLQESGPGLVKPSQSLSLTCSVT | DIVLTQSPASLAVSLGQRATISCRASKSVS |
| | GYSITSGYYWNWIRQFPGNKLEWM | TSGSNFLHWYQQKPGQPPKLLIYFASNLE |
| | GYISYDGSFNYNPSLKNRLSITRDTS | SGVPARFSGSGSGTDFTLNIHPVEEEDAA |
| | MNQFFLKLNSVTSEDTATYYCARA | TYYCQHSRELPHTFGAGTKLELKRADAA |
| | EIYYDYDGDFDYWGQGTTLTVSSA | PTVSIFPPSSEQLTSGGASVVCFLNNFYPK |
| | KTTAPSVYPLAPVCGDTTGSSVTLG | DINVKWKIDGSERQNGVLNSWTDQDSKD |
| | CLVKGYFPEPVTLTWNSGSLSSGVH | STYSMSSTLTLTKDEYERHNSYTCEATHK |
| | TFPAVLQSDLYTLSSSVTVTSSTWPS | TSTSPIVKSFNRNEC (SEQ ID NO: 101) |
| | QSITCNVAHPASSTKVDKKIEPRGPT | |
| | IKPCPPCKCPAPNLLGGPSVFIFPPKI | |
| | KDVLMISLSPIVTCVVVDVSEDDPD | |
| | VQISWFVNNVEVHTAQTQTHREDY | |
| | NSTLRVVSALPIQHQDWMSGKEFK | |
| | CKVNNKDLPAPIERTISKPKGSVRAP | |
| | QVYVLPPPEEEMTKKQVTLTCMVT | |
| | DFMPEDIYVEWTNNGKTELNYKNT | |
| | EPVLDSDGSYFMYSKLRVEKKNWV | |
| | ERNSYSCSVVHEGLHNHHTTKSFSR | |
| | TPGK (SEQ ID NO: 100) | |
| I23RB76 | EVQLQQSGPELVKPGASVKMSCKA | DIVLTQSPATLCVTPGETVSLSCRASQNIY |
| | SGYTFTDYYMNWMKQSHGKSLEW | KNLHWFQQKSHRSPRLLIKYASDSISGIPS |
| | IGYISPKNGRTGYNQKFKGKATLTV | RFTGSGSGTDYTLSINSVKPEDEGIYYCLQ |
| | DKSSTTAYMELRSLTSEDSAVYYCA | GYSTPWTFGGGTKLEIKRADAAPTVSIFP |
| | KSNGYYALDYWGQGTTVTVSSAKT | PSSEQLTSGGASVVCFLNNFYPKDINVKW |
| | TAPSVYPLAPVCGDTTGSSVTLGCL | KIDGSERQNGVLNSWTDQDSKDSTYSMS |
| | VKGYFPEPVTLTWNSGSLSSGVHTF | STLTLTKDEYERHNSYTCEATHKTSTSPIV |
| | PAVLQSDLYTLSSSVTVTSSTWPSQS | KSFNRNEC (SEQ ID NO: 103) |
| | ITCNVAHPASSTKVDKKIEPRGPTIK | |
| | PCPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 102) | |
| I23RB77 | DVQLQESGPGLVAPSQSLSITCTVSG | DIVMTQSPASLALSLGQRATISCRASESV |
| | FSLSRYSVHWVRQPPGKGLEWLGM | DSYGNSFMHWYQQKPGQPPKLLIYLASN |
| | IWGSGSTDYNSALKSRLSINKDNSK | LESGVPARFSGSGSRTDFTLTIDPVEADD |
| | SQVFLKMNSLQTDDTAMYYCARV | AAIYYCQQNNEEPNTFGAGTKLEMKRAD |
| | YYGGSPYAMDYWGQGTTVTVSSA | AAPTVSIFPPSSEQLTSGGASVVCFLNNFY |
| | KTTAPSVYPLAPVCGDTTGSSVTLG | PKDINVKWKIDGSERQNGVLNSWTDQDS |
| | CLVKGYFPEPVTLTWNSGSLSSGVH | KDSTYSMSSTLTLTKDEYERHNSYTCEAT |
| | TFPAVLQSDLYTLSSSVTVTSSTWPS | HKTSTSPIVKSFNRNEC (SEQ ID NO: 105) |
| | QSITCNVAHPASSTKVDKKIEPRGPT | |
| | IKPCPPCKCPAPNLLGGPSVFIFPPKI | |
| | KDVLMISLSPIVTCVVVDVSEDDPD | |
| | VQISWFVNNVEVHTAQTQTHREDY | |
| | NSTLRVVSALPIQHQDWMSGKEFK | |
| | CKVNNKDLPAPIERTISKPKGSVRAP | |
| | QVYVLPPPEEEMTKKQVTLTCMVT | |
| | DFMPEDIYVEWTNNGKTELNYKNT | |
| | EPVLDSDGSYFMYSKLRVEKKNWV | |
| | ERNSYSCSVVHEGLHNHHTTKSFSR | |
| | TPGK (SEQ ID NO: 104) | |
| I23RB78 | EFQLQQSGGGLVKPGGSLKLSCAAS | DIVMTQSPASLAVSLGQRATISCRASESV |
| | GFTFSNYTMSWVRQTPEKRLKWVA | DSYGISFMHWYQQKPGQPPKLLIYRASNL |
| | CISSGGGNTYYPDSVKGRFTISRDN | ESGIPARFSGSGSRTDFTLTINPVEADDVA |
| | ARNTLYLQMSSLRSEDTAMYYCAR | TYYCQQSNEDPWTFGAGTGLEIKRADAA |
| | RNYRRAMDYWGQGTSVTVSSAKT | PTVSIFPPSSEQLTSGGASVVCFLNNFYPK |
| | TAPSVYPLAPVCGDTTGSSVTLGCL | DINVKWKIDGSERQNGVLNSWTDQDSKD |
| | VKGYFPEPVTLTWNSGSLSSGVHTF | STYSMSSTLTLTKDEYERHNSYTCEATHK |
| | PAVLQSDLYTLSSSVTVTSSTWPSQS | TSTSPIVKSFNRNEC (SEQ ID NO: 107) |
| | ITCNVAHPASSTKVDKKIEPRGPTIK | |
| | PCPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 106) | |
| I23RB79 | DVQLQESGPELVRPGASVKMSCKA | DIQMTQSPKFLHVSAGDRVTITCKASQSV |
| | SGYTFTDYYMNWVKQSHGKSLEWI | INDVAWYQQKPGQSPKLLIYYASNRYTG |
| | GYIYPNNGDTVYNQKFKGKATLTV | VPDRFTGSGYGTDFTFTISTVQAEDLAIYF |
| | DKSSNTAYMELRSLTSEDSAVYYC | CQQDYSSPPTFGAGTRLEIKRADAAPTVSI |
| | ARGTLTGMYYFDYWGQGTSVTVSS | FPPSSEQLTSGGASVVCFLNNFYPKDINV |
| | AKTTAPSVYPLAPVCGDTTGSSVTL | KWKIDGSERQNGVLNSWTDQDSKDSTYS |
| | GCLVKGYFPEPVTLTWNSGSLSSGV | MSSTLTLTKDEYERHNSYTCEATHKTSTS |
| | HTFPAVLQSDLYTLSSSVTVTSSTW | PIVKSFNRNEC (SEQ ID NO: 109) |
| | PSQSITCNVAHPASSTKVDKKIEPRG | |
| | PTIKPCPPCKCPAPNLLGGPSVFIFPP | |
| | KIKDVLMISLSPIVTCVVVDVSEDDP | |
| | DVQISWFVNNVEVHTAQTQTHRED | |
| | YNSTLRVVSALPIQHQDWMSGKEF | |
| | KCKVNNKDLPAPIERTISKPKGSVR | |
| | APQVYVLPPPEEEMTKKQVTLTCM | |
| | VTDFMPEDIYVEWTNNGKTELNYK | |
| | NTEPVLDSDGSYFMYSKLRVEKKN | |
| | WVERNSYSCSVVHEGLHNHHTTKS | |
| | FSRTPGK (SEQ ID NO: 108) | |
| I23RB80 | DVQLQESGPGLVQPSQSLSITCTVSG | DIVMTQSQKFMSTSEGDRVNITCKASQNI |
| | FSLTSYGVHWVRQSPGKGLEWLGV | RTAVAWYQQKPGQSPKPLIYLASNRHTG |
| | IWSGVSTDYNAAFISRLSISKDNSKS | VPDRFTGSGSGTDFTLTISNVQSEDLAEYF |
| | QVFFKMNSLQADDTAIYYCARSRG | CLQHWNYPLTFGAGTKLELKRADAAPTV |
| | SYYFDYWGQGTTLTVSSAKTTAPS | SIFPPSSEQLTSGGASVVCFLNNFYPKDIN |
| | VYPLAPVCGDTTGSSVTLGCLVKG | VKWKIDGSERQNGVLNSWTDQDSKDST |
| | YFPEPVTLTWNSGSLSSGVHTFPAV | YSMSSTLTLTKDEYERHNSYTCEATHKTS |
| | LQSDLYTLSSSVTVTSSTWPSQSITC | TSPIVKSFNRNEC (SEQ ID NO: 111) |
| | NVAHPASSTKVDKKIEPRGPTIKPCP | |
| | PCKCPAPNLLGGPSVFIFPPKIKDVL | |
| | MISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 110) | |
| I23RB81 | DVQLQESGPGLVAPSQSLSITCTVSG | DIVMTQSHKFMSTSVGDRVSIICKASQDV |
| | FSLTSYAINWVRQPPGKGLEWLGVI | STAVAWYQQKPGQSPKLLIYSASYRYTG |
| | WTGGGTNYNSALKSRLSISKDNSKS | VPDRFTGSGSGTDFTFTISSVQAEDLAVY |
| | QLFLKMNSLQTDDTARYYCARNRD | YCQQHYSTPLTFGAGTRLEIKRADAAPTV |
| | YSNYRWYFDVWGAGTSVTVSSAKT | SIFPPSSEQLTSGGASVVCFLNNFYPKDIN |
| | TAPSVYPLAPVCGDTTGSSVTLGCL | VKWKIDGSERQNGVLNSWTDQDSKDST |
| | VKGYFPEPVTLTWNSGSLSSGVHTF | YSMSSTLTLTKDEYERHNSYTCEATHKTS |
| | PAVLQSDLYTLSSSVTVTSSTWPSQS | TSPIVKSFNRNEC (SEQ ID NO: 113) |
| | ITCNVAHPASSTKVDKKIEPRGPTIK | |
| | PCPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 112) | |
| I23RB82 | QVQLKESGPGLVAPSQSLSITCTVSG | QIVLSQSHKFMSTSVGDRVSITCKASQDV |
| | FSLTNYALHWFRQSPGKGLEWLGVI | STAVAWYQQKPGQSPKLLIYSAFYRYTG |
| | WSDGSTDYNSPFISRLSISKDNSKSQ | VPDRFTGSGSGTDFTFTISSVQAEDLAVY |
| | VFFKMNSLQADDTAIYYCARSRSN | YCQQHYSTPYTFGGGTKLEIKRADAAPT |
| | YGRWYFDVWGAGTTLTVSSAKTTA | VSIFPPSSEQLTSGGASVVCFLNNFYPKDI |
| | PSVYPLAPVCGDTTGSSVTLGCLVK | NVKWKIDGSERQNGVLNSWTDQDSKDS |
| | GYFPEPVTLTWNSGSLSSGVHTFPA | TYSMSSTLTLTKDEYERHNSYTCEATHKT |
| | VLQSDLYTLSSSVTVTSSTWPSQSIT | STSPIVKSFNRNEC (SEQ ID NO: 115) |
| | CNVAHPASSTKVDKKIEPRGPTIKPC | |
| | PPCKCPAPNLLGGPSVFIFPPKIKDV | |
| | LMISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 114) | |
| I23RB83 | DVQLQESGPGLVAPSQSLSITCTVSG | DIQMTQSPASQSASLGESVTITCLASQTIG |
| | FSLTNYDVHWVRQSPGKGLEWLGV | SWLAWYQQKPGKSPQLLIYAATSLADGV |
| | IWSDGSTDYNAAFISRLSFSKDNSKR | PSRFSGSGSGTKFSFKISSLQAEDFVSYYC |
| | QVFFKMNSLQPDDTAIYYCARNNY | QQLYSSPLTFGGGTKLEIKRADAAPTVSIF |
| | YRYGRNPMDYWGQGTTVTVSSAK | PPSSEQLTSGGASVVCFLNNFYPKDINVK |
| | TTAPSVYPLAPVCGDTTGSSVTLGC | WKIDGSERQNGVLNSWTDQDSKDSTYS |
| | LVKGYFPEPVTLTWNSGSLSSGVHT | MSSTLTLTKDEYERHNSYTCEATHKTSTS |
| | FPAVLQSDLYTLSSSVTVTSSTWPSQ | PIVKSFNRNEC (SEQ ID NO: 117) |
| | SITCNVAHPASSTKVDKKIEPRGPTI | |
| | KPCPPCKCPAPNLLGGPSVFIFPPKIK | |
| | DVLMISLSPIVTCVVVDVSEDDPDV | |
| | QISWFVNNVEVHTAQTQTHREDYN | |
| | STLRVVSALPIQHQDWMSGKEFKC | |
| | KVNNKDLPAPIERTISKPKGSVRAPQ | |
| | VYVLPPPEEEMTKKQVTLTCMVTD | |
| | FMPEDIYVEWTNNGKTELNYKNTE | |
| | PVLDSDGSYFMYSKLRVEKKNWVE | |
| | RNSYSCSVVHEGLHNHHTTKSFSRT | |
| | PGK (SEQ ID NO: 116) | |
| I23RB85 | DVQLQESGPGLVAPSQSLSITCTVSG | DIVLTQSPLSLFVSLGDQASISCRSSQSLV |
| | FSFNTYAITWVRQPPGRGLEWLGVL | HSNGNTYLHWYLQKPGQSPKLLIYKVSN |
| | WPGGGISYNLALKSRLTISRDSSKSQ | RFSGVPDRFSGSGSGTDFTLKISRVEAEDL |
| | VFLEMNSLQTDDTARYYCARNWR | GVYFCSQSTHVPWTFGGGTRLEIKRADA |
| | HYDGSYAMDFWGQGTSVTVSSAKT | APTVSIFPPSSEQLTSGGASVVCFLNNFYP |
| | TAPSVYPLAPVCGDTTGSSVTLGCL | KDINVKWKIDGSERQNGVLNSWTDQDSK |
| | VKGYFPEPVTLTWNSGSLSSGVHTF | DSTYSMSSTLTLTKDEYERHNSYTCEATH |
| | PAVLQSDLYTLSSSVTVTSSTWPSQS | KTSTSPIVKSFNRNEC (SEQ ID NO: 119) |
| | ITCNVAHPASSTKVDKKIEPRGPTIK | |
| | PCPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 118) | |
| I23RB86 | EVQLQQSGAELVKPGASVKLSCKA | DIVMTQSPSSMSVSLGDTVSITCHASQGIS |
| | SGYTFTSYWLHWVKQRPGQGLEWI | SNIGWLQQKPGKSFKGLIYHGTNLEDEVP |
| | GEIDPSDSYTYYNQKFKGKATLTVD | SRFSGSGSGADYSLTIGSLESEDFADYYC |
| | KSSSTAYMQLSSLTSEDSAVYYCAR | VQYAQFPWTFGAGTKLEMKRADAAPTV |
| | SPYDYDWGAYWGQGTSVTVSSAK | SIFPPSSEQLTSGGASVVCFLNNFYPKDIN |
| | TTAPSVYPLAPVCGDTTGSSVTLGC | VKWKIDGSERQNGVLNSWTDQDSKDST |
| | LVKGYFPEPVTLTWNSGSLSSGVHT | YSMSSTLTLTKDEYERHNSYTCEATHKTS |
| | FPAVLQSDLYTLSSSVTVTSSTWPSQ | TSPIVKSFNRNEC (SEQ ID NO: 121) |
| | SITCNVAHPASSTKVDKKIEPRGPTI | |
| | KPCPPCKCPAPNLLGGPSVFIFPPKIK | |
| | DVLMISLSPIVTCVVVDVSEDDPDV | |
| | QISWFVNNVEVHTAQTQTHREDYN | |
| | STLRVVSALPIQHQDWMSGKEFKC | |
| | KVNNKDLPAPIERTISKPKGSVRAPQ | |
| | VYVLPPPEEEMTKKQVTLTCMVTD | |
| | FMPEDIYVEWTNNGKTELNYKNTE | |
| | PVLDSDGSYFMYSKLRVEKKNWVE | |
| | RNSYSCSVVHEGLHNHHTTKSFSRT | |
| | PGK (SEQ ID NO: 120) | |
| I23RB87 | EFQLQQSGPELVKPGTSVKMSCKAS | DIVMTQTTSSLSASLGDRVTISCRASQDIS |
| | GYTFTNYVMHWVKQKPGQGLEWI | NYLNWYQQKPDGTVKLLIYYTSRLHSGV |
| | GYINPYDASPKYNEKFKGKAILTSD | PSRFSGSGSGTDFSLTISNLEQEDIATYFC |
| | KSSSTAYMELSSLTSEDSAVYYCTR | QQGITLPWTFGAGTKLELKRADAAPTVSI |
| | DAKLDYFDYWGQGTTLTVSSAKTT | FPPSSEQLTSGGASVVCFLNNFYPKDINV |
| | APSVYPLAPVCGDTTGSSVTLGCLV | KWKIDGSERQNGVLNSWTDQDSKDSTYS |
| | KGYFPEPVTLTWNSGSLSSGVHTFP | MSSTLTLTKDEYERHNSYTCEATHKTSTS |
| | AVLQSDLYTLSSSVTVTSSTWPSQSI | PIVKSFNRNEC (SEQ ID NO: 123) |
| | TCNVAHPASSTKVDKKIEPRGPTIKP | |
| | CPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 122) | |
| I23RB91 | EFQLQQFGPELLKPGASVKISCKAS | DIKMTQSPSSMYASLGERVTITCKASQDI |
| | GYTFTDYIMHWVKQSHGKSLEWIG | NSYLSWCQQKPGKSPKALIYRANSLVDG |
| | GISPNYGGTGYNGKFKDKATLTVD | VPSRFSGSGSGQDYSLTISSLEYEDMGIYY |
| | KSSSTAYMELRSLTSEDSAVYYCAR | CLQYDEFPYTFGGGTKLEMKRADAAPTV |
| | FNNYYYFDYWGQGTTLTVSSAKTT | SIFPPSSEQLTSGGASVVCFLNNFYPKDIN |
| | APSVYPLAPVCGDTTGSSVTLGCLV | VKWKIDGSERQNGVLNSWTDQDSKDST |
| | KGYFPEPVTLTWNSGSLSSGVHTFP | YSMSSTLTLTKDEYERHNSYTCEATHKTS |
| | AVLQSDLYTLSSSVTVTSSTWPSQSI | TSPIVKSFNRNEC (SEQ ID NO: 125) |
| | TCNVAHPASSTKVDKKIEPRGPTIKP | |
| | CPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 124) | |
| I23RB92 | EVQLQQSGPEVVKPGASVKISCKAS | DIKMTQSPASLAVSLGQRATISCRASQSV |
| | GYTFTDHTMDWVKQSHGKNLEWI | STSKYDYIHWYQQKSGQPPRLLIKYASNL |
| | GYIYPKSGGTGYNQKFKNKATLTV | ESGVPARFSGSGSGTDFTLNIHPVEEEDTA |
| | DKSSSTAYMEFHSLTSDDSAVYYCA | TYYCQHTWEIPYTFGSGTKLELKRADAA |
| | RWGLRRDGFVYWGQGTLVTVSAA | PTVSIFPPSSEQLTSGGASVVCFLNNFYPK |
| | KTTAPSVYPLAPVCGDTTGSSVTLG | DINVKWKIDGSERQNGVLNSWTDQDSKD |
| | CLVKGYFPEPVTLTWNSGSLSSGVH | STYSMSSTLTLTKDEYERHNSYTCEATHK |
| | TFPAVLQSDLYTLSSSVTVTSSTWPS | TSTSPIVKSFNRNEC (SEQ ID NO: 127) |
| | QSITCNVAHPASSTKVDKKIEPRGPT | |
| | IKPCPPCKCPAPNLLGGPSVFIFPPKI | |
| | KDVLMISLSPIVTCVVVDVSEDDPD | |
| | VQISWFVNNVEVHTAQTQTHREDY | |
| | NSTLRVVSALPIQHQDWMSGKEFK | |
| | CKVNNKDLPAPIERTISKPKGSVRAP | |
| | QVYVLPPPEEEMTKKQVTLTCMVT | |
| | DFMPEDIYVEWTNNGKTELNYKNT | |
| | EPVLDSDGSYFMYSKLRVEKKNWV | |
| | ERNSYSCSVVHEGLHNHHTTKSFSR | |
| | TPGK (SEQ ID NO: 126) | |
| I23RB93 | QVQLKESGPGLVQPSQSLSITCTVSG | DIKMTQSPATLSVTPGDSVSLFCRASQSIS |
| | FSLTSYGVHWVRQSPGKGLEWLGV | NNLHWYQQKSHESPRLLIKYASQSISGIPS |
| | IWSGVSTDYNAAFISRLSISKDNSKS | RFSGSGSGTDFTLSINSVETEDFGMYFCQ |
| | QVFFKMNSLQADDTAIYYCARSRG | QSNSWPLTFGGGTKLEIKRADAAPTVSIF |
| | SYYFDYWGQGTTLTVSSAKTTAPS | PPSSEQLTSGGASVVCFLNNFYPKDINVK |
| | VYPLAPVCGDTTGSSVTLGCLVKG | WKIDGSERQNGVLNSWTDQDSKDSTYS |
| | YFPEPVTLTWNSGSLSSGVHTFPAV | MSSTLTLTKDEYERHNSYTCEATHKTSTS |
| | LQSDLYTLSSSVTVTSSTWPSQSITC | PIVKSFNRNEC (SEQ ID NO: 129) |
| | NVAHPASSTKVDKKIEPRGPTIKPCP | |
| | PCKCPAPNLLGGPSVFIFPPKIKDVL | |
| | MISLSPIVTCVVVDVSEDDPDVQIS | |
| | WFVNNVEVHTAQTQTHREDYNSTL | |
| | RVVSALPIQHQDWMSGKEFKCKVN | |
| | NKDLPAPIERTISKPKGSVRAPQVYV | |
| | LPPPEEEMTKKQVTLTCMVTDFMPE | |
| | DIYVEWTNNGKTELNYKNTEPVLD | |
| | SDGSYFMYSKLRVEKKNWVERNSY | |
| | SCSVVHEGLHNHHTTKSFSRTPGK | |
| | (SEQ ID NO: 128) | |
| I23RB94 | DVQLQESGPELKKPGETVKISCKAS | DIVMTQSHKFMSTSVGDRVSITCKASQD |
| | GYTFTNYGMNWVRQAPGKSLKWM | VNTAVAWYQQKPGQSPKLLIYSAFYRYP |
| | GWINTNTGEPAYAEEFKGRFAFSLE | GVPDRFTGSGSGTDFTFTINSVQAEDLAV |
| | TSASTAYLQINNLKNEDMATYFCAR | YCCQQHYTSPLTFGDGTRLEIKRADAAPT |
| | VRQTARATRGYFDVWGTGTTVTVS | VSIFPPSSEQLTSGGASVVCFLNNFYPKDI |
| | SAKTTAPSVYPLAPVCGDTTGSSVT | NVKWKIDGSERQNGVLNSWTDQDSKDS |
| | LGCLVKGYFPEPVTLTWNSGSLSSG | TYSMSSTLTLTKDEYERHNSYTCEATHKT |
| | VHTFPAVLQSDLYTLSSSVTVTSST | STSPIVKSFNRNEC (SEQ ID NO: 131) |
| | WPSQSITCNVAHPASSTKVDKKIEP | |
| | RGPTIKPCPPCKCPAPNLLGGPSVFIF | |
| | PPKIKDVLMISLSPIVTCVVVDVSED | |
| | DPDVQISWFVNNVEVHTAQTQTHR | |
| | EDYNSTLRVVSALPIQHQDWMSGK | |
| | EFKCKVNNKDLPAPIERTISKPKGSV | |
| | RAPQVYVLPPPEEEMTKKQVTLTC | |
| | MVTDFMPEDIYVEWTNNGKTELNY | |
| | KNTEPVLDSDGSYFMYSKLRVEKK | |
| | NWVERNSYSCSVVHEGLHNHHTTK | |
| | SFSRTPGK (SEQ ID NO: 130) | |
| I23RB95 | EVKLVESGGGLVQPGSSMKLSCTAS | DIVLTQSHKFMSTSVGDRVSITCKASQDV |
| | GFTFSDYYMAWVRQVPEKGLEWV | GTAVAWYQQKPGQSPKLLIYWASTRHTG |
| | ANINYDGSNTYYLDSLKSRFIISRDN | VPDRFTGSGSGTDFTLTISNVQSEDLADY |
| | AKNILYLQMSSLKSEDTATYYCARE | FCQQYSTYPYTVGGGTKLEIKRADAAPT |
| | GYDEAMDYWGQGTTLTVSSAKTT | VSIFPPSSEQLTSGGASVVCFLNNFYPKDI |
| | APSVYPLAPVCGDTTGSSVTLGCLV | NVKWKIDGSERQNGVLNSWTDQDSKDS |
| | KGYFPEPVTLTWNSGSLSSGVHTFP | TYSMSSTLTLTKDEYERHNSYTCEATHKT |
| | AVLQSDLYTLSSSVTVTSSTWPSQSI | STSPIVKSFNRNEC (SEQ ID NO: 133) |
| | TCNVAHPASSTKVDKKIEPRGPTIKP | |
| | CPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 132) | |
| I23RB98 | EFQLQQSGPELVKPGASVKMSCKAS | DIVLTQSPAIMSASLGERVTMTCTASSSVS |
| | GYTFTNYYMHWVKQSHGKTIEWIG | SSYLHWYQQKPGSSPKLWIYSTSNLASG |
| | YIYPNSGDTSYNQKFKGKATLTVDK | VPARFSGSGSGTSYSLTISSMEAEDAATY |
| | SSSTAYMELRSLTSEDSAVYTCARF | YCHQYHRSPFTFGSGTKLEMKRADAAPT |
| | GGFYDAMDYWGQGTTVTVSSAKT | VSIFPPSSEQLTSGGASVVCFLNNFYPKDI |
| | TAPSVYPLAPVCGDTTGSSVTLGCL | NVKWKIDGSERQNGVLNSWTDQDSKDS |
| | VKGYFPEPVTLTWNSGSLSSGVHTF | TYSMSSTLTLTKDEYERHNSYTCEATHKT |
| | PAVLQSDLYTLSSSVTVTSSTWPSQS | STSPIVKSFNRNEC (SEQ ID NO: 135) |
| | ITCNVAHPASSTKVDKKIEPRGPTIK | |
| | PCPPCKCPAPNLLGGPSVFIFPPKIKD | |
| | VLMISLSPIVTCVVVDVSEDDPDVQI | |
| | SWFVNNVEVHTAQTQTHREDYNST | |
| | LRVVSALPIQHQDWMSGKEFKCKV | |
| | NNKDLPAPIERTISKPKGSVRAPQVY | |
| | VLPPPEEEMTKKQVTLTCMVTDFM | |
| | PEDIYVEWTNNGKTELNYKNTEPVL | |
| | DSDGSYFMYSKLRVEKKNWVERNS | |
| | YSCSVVHEGLHNHHTTKSFSRTPGK | |
| | (SEQ ID NO: 134) | |

TABLE 5
VH and VL AA Sequences of Exemplary Immunization Based IL-23R Antibodies

| Name | VH AA Seq | VL AA Seq |
|---|---|---|
| I23RB101 | EVQLQQSGAEVKKPGSSVKVSCKA | DIVMTQSHKFMSTSIGDRVSITCKTSQDV |
| | SGGTFSSYAISWVRQAPGQGLEWM | RTAVAWYQQKPGQSPKLLIYSASYRYIG |
| | GWINPNSGGTNYAQKFQGRVTMTR | VPDRFTGSGSGTDFTFTISDVQAEDLALY |
| | DTSISTAYMELSRLRSDDTAVYYCA | YCQQHYSTPYTFGGGTKLEIK (SEQ ID |
| | RGYRYGPLDGFDIWGQGTTLTVSS | NO: 137) |
| | (SEQ ID NO: 136) | |
| I23RB102 | QVQLQQPGAELVKPGASVKVSCKA | DIQMTQSPASLSVSVGETVTITCRASENIY |
| | SGYTFTKYWINWMNQRPGQGLEWI | SHITWYQLKQGKSPQLLVYASTNLADDV |
| | GNIYPGSISTNYNEKFKSKVTLTVDI | PSRFSGSGSGTQYSLKINSLLSEDFGTYYC |
| | SSSTAYMQLSSLTSDDSAVYYCARG | QHFWGTPWTFGGGTKLEMK (SEQ ID |
| | DFYALDYWGQGTTLTVSS (SEQ ID | NO: 139) |
| | NO: 138) | |
| I23RB104 | EVKLVESGGGLVKPGGSLKLSCAAS | DIQMTQSPASLSVSVGETVTITCRASENIY |
| | GFTFSSYAMSWVRQTPEKRLDWVA | SNLAWYQQKQGKSPQLLVYAATNLADG |
| | SISTGGSTYYPDSVKGGFTISRDIAR | VPSRFSGSGSGTQYSLKINSLQSEDFGSYY |
| | NTLSLQMTSLRSEDTAMYYCARGP | CQHFWGTPPTFGSGTKLEIK (SEQ ID |
| | HYYGGSYPFAYWGQGTTLTVSS | NO: 141) |
| | (SEQ ID NO: 140) | |
| I23RB144 | EVQLQQSGPELVKPGASVKMSCKA | DIVMTQSPLTLSVIIGQSASISCKSSQSLLD |
| | SGYTFTSYIIHWVKQKPGQGLEWIG | SDGKTYLNWLLQRPGQSPKRLMYLVSKL |
| | YIDPYNDDTKYNEKFKGKATLTSD | DSGVPDRFTGSGSGTDFTLKISRVEAEDL |
| | KSSSTAYMEFSSLTSEDSAVYYCAR | GVYYCWQGIHFPLTFGSGTRLEIK (SEQ |
| | LGSINYYDGSYFDYWGQGTSVTVSS | ID NO: 143) |
| | (SEQ ID NO: 142) | |
| I23RB148 | DVQLQESGPDLVKPSQSLSLTCTVT | DIVMTQSHKFMSTSVGDRVSLTCKASQE |
| | GYSITSGYNWHWIRQFPGNKLEWM | VRTAVAWYQQKPGQPPKLLIYSASYRYT |
| | GYIHSSGTTNYSPSLRSRLSITRDTSK | GVPDRFTGSGSGTDFTFTIISVQAEDLAVY |
| | NQFFLQLNSVTTEDTATYYCTRQLG | YCQQHYTTPYSFGSGTKLEIK (SEQ ID |
| | LKAMEYWGQGTTLTVSS (SEQ ID | NO: 145) |
| | NO: 144) | |
| I23RB149 | DVQLQESGPDLVKPSQSLSLTCTVT | DVQMIQSPLSLPVSLGDQASISCRSSQSLV |
| | GYSITSGYNWHWIRQFPGNKLEWM | HSNGNTYLHWYLQKPGQSPKLLIYKVSN |
| | GYIHFSDRTNYNPSLKSRISITRDTSK | RFSGVPDRFSGSGSGTDFTLKISRVEAEDL |
| | NQFFLQLNSVTSEDTATYYCSRQLG | GVYFCSQSTHVPLTFGAGTRLEIK (SEQ |
| | LKALDYWGQGTTLTVSS (SEQ ID | ID NO: 147) |
| | NO: 146) | |
| I23RB150 | QIQLVQSGPELKKPGETVKISCKASA | DIVMTQSHKFMSTSVGGRVSITCKASQD |
| | YTFTNYGMNWVKQAPGKGLKWM | VSTAVAWYQQKPGQSPRLLIYSASYRYT |
| | GWIKTNTGEPTYAEEFKGRFAFSLE | GVPDRFTGSGSGTDFTFTISSVQAEDLAV |
| | TSASTAYLQINNLKNEDTATYFCAR | FYCQQHYSTPYTFGSGTRLEIK (SEQ ID |
| | SRQLGLRYYFDYWGQGTTLTVSS | NO: 149) |
| | (SEQ ID NO: 148) | |
| I23RB153 | EVKLVESGGGLVQPGGSLKLSCAAS | DIVMTQSHRFMSTSVGERVSITCKASQDV |
| | GFTFSSDTMSWVRQTPEKRLELVAE | GNAVAWYQQKPGQSPKLLIYWTSTRHN |
| | ISSNGGSTYHIDTVKGRFTISRDNAK | GVPDRFTGSGSGTDFTLTISNVQGEDLAD |
| | NTLYLQMSSLKSEDTAMYYCASPG | YFCQQYNRYPLTFGSGTKLEIK (SEQ ID |
| | YPWFAYWGQGTTVTVSA (SEQ ID | NO: 151) |
| | NO: 150) | |
| I23RB155 | QVQLKESGPGLVKPSQSLSLTCSVT | QIVLSQSPASLAVSLGQRATISCRTSESVD |
| | GYSITSGHSWNWIRQFPGNKLEWM | TYGNSFIYWYQQKPGQPPKLLIYRASNLE |
| | GYISYDGTNDYNPSLKNRISITRDTS | SGIPARFSGSGSRTDFTLTINPVEADDVAT |
| | KNQFFLNLNSVTTEDTVTYYCARG | YYCQQGNKDPYTFGSGTKLEMK (SEQ ID |
| | GGYYGGTYRVFADWGQGTTLTVSS | NO: 153) |
| | (SEQ ID NO: 152) | |
| I23RB156 | QVQLKESGPGLVAPSQSLSITCTVSG | DIVMTQSHKFMSTSVGDRVSITCKASQD |
| | FSLTSYAINWVRQPPGKGLEWLGVI | VSTAVAWYQQKPGRSPKLLIYSAFYRYT |
| | WTGGGTNYNSVFKSRLSISKDNSKS | GVPDRFTGSGSGTDFTFTIGSVQAEDLAV |
| | QIFLKMISLQTDDTARYYCARNRDY | YYCQQHYSIPLTFGAGTKLELK (SEQ ID |
| | SNYRWYFDVWGAGTTLTVSS (SEQ | NO: 155) |
| | ID NO: 154) | |
| I23RB157 | EVKLVESGGGSVKPGGSLKLSCAAS | DIVMTQSPASLAVSLGQRATISCRASESV |
| | GFTFSRYTMSWVRQTPEKRLEWVA | DNYGISFMNWFQQKPGQPPKLLIYAASN |
| | YISTGGGDTYYPDSVKGRFTISRDN | QGSGVPARFSGSGSGTDFSLNIHPMEEDD |
| | ARNTLYLQMSSLRSEDTAMYYCAR | TAMYFCQQSKEVPWTFGAGTKLELK |
| | RTSYSYYNYVFDYWGQGTTLTVSS | (SEQ ID NO: 157) |
| | (SEQ ID NO: 156) | |
| I23RB159 | EVQLQQSGGGLVKPGGSLKLSCAA | DIVMTQTTSSLSASLGDRVTISCRASQDIS |
| | SGFTFSDYGMAWVRQTPRKGPEWI | NYLNWYQQKPDGTVKLLIYYTSRLHSGV |
| | AFISNLAYSVYYSDTVTGRFTISREN | PSRFSGSGSGTDYSLTISNLEQEDIATYFC |
| | AKNTLYLEMSSLRSEDTAMYYCAR | QQGNTLPWTFGAGTKLELK (SEQ ID |
| | HGDYRNYGFAYWGLGTTVTVSS | NO: 159) |
| | (SEQ ID NO: 158) | |
| I23RB160 | QVQLKESGPDLVKPSQSLSLTCTVT | DIVMTQSHKFMSTSVGDRVSITCKASQD |
| | GYSITSGYNWHWIRQFPGNKLEWM | VRTAVAWYQQKPGQSPKLLIHSASYRYT |
| | GYIHYSSGTIYNPSLKSRISITRDTSK | GVPDRFTGSGSGTDFTFTISSVQVEDLAV |
| | NQFFLQLNSVTTEDTGTYYCARQLG | YYCQQHYTTPYTFGSGTKLEIK (SEQ ID |
| | LKAMDYWGQGTSVTVSS (SEQ ID | NO: 161) |
| | NO: 160) | |
| I23RB161 | EFQLQQSGPELVKPGASVKISCKAS | DIVMTQSPAILSASPGEKVTMTCRASSSV |
| | VYAFSSSWMNWVKQRPGKGLEWI | SYMHWYQQKPGSSPKPWIYATSDLASGA |
| | GRIYPGDGDTNYNGKFKGKATLTA | PARFSGSGSGTSYSLTISRVEAEDAATYY |
| | DKSSSTAYMQLSSLTSEDSAVYFCA | CQQWSSNPPTFGGGTKLEIK (SEQ ID |
| | RRYDFGSAMDYWGQGTSVTVSS | NO: 163) |
| | (SEQ ID NO: 162) | |
| I23RB162 | EFQLQQSGPELVKPGASVKMSCKAS | DIVMTQSQKFMSTSVGDRVSVTCKASQN |
| | GYTFTNYVMHWVKQKPGQGLEWI | VGTNVAWYQQKPGQSPKALIYSASYRYS |
| | GYINPYNDGTKYNEKFKGKATLTS | GVPDRFTGSGSGTDFTLTISNVQSEDLAE |
| | DKSSSTAYMELSSLTSEDSAVYYCA | YFCQQYNSYPFTFGSGTKLEIK (SEQ ID |
| | RSRTGTRWYFDVWGAGTTVTVSS | NO: 165) |
| | (SEQ ID NO: 164) | |
| I23RB165 | EVQLQQSGAELVRPGASVKLSCTAS | DIVMTQSPASLAVSLGQRATISCRASKSV |
| | GFNIKDSLMHWVKQRPEQGLEWIG | STSGYSYMHWYQQKPGQPPKLLIYLASN |
| | WIDPEDGKTKYAPKFQDKATITADT | LESGVPARFSGSGSGTDFTLNIHPVEEEDA |
| | SSNTAYLQLSSLTSEDTAIYYCARRE | ATYYCQHSRELPLTFGAGTKLEMK (SEQ |
| | VLDYFDYWGQGTTLTVSS (SEQ ID | ID NO: 167) |
| | NO: 166) | |
| I23RB166 | DVQLQESGPGLVKPSQTVSLTCTVT | DIVMTQSHKFMSTSVGARVSITCKASQD |
| | GYSITNGNHWWNWIRQVSGSKLEW | VSTAVAWYQQKPGQSPKLLIYSASYRYT |
| | IGYIRSSGSTDSNPSLKSRISITRDTSK | GVPDRFTGSGSGTDFTFTISSVQAEDLAV |
| | NQLFLQLNSVTAEDIATYYCARERL | YYCQQHYSTPRTFGGGTKLEIK (SEQ ID |
| | RLRWSYFDYWGQGTTLTVSS (SEQ | NO: 169) |
| | ID NO: 168) | |
| I23RB168 | QIQLVQSGPELVKPGASVKMSCKTS | DIQMTQSSSSFSVSLGDRVTITCKASEDLY |
| | GYTFTDYYINWVKQRPGQGLEWIG | NRLAWYQQKPGNAPRLLISGATSLGTGV |
| | WIYPAINNTNYNEKFKGKATLTVDT | LSRFSGSGSGKDYTLSITSLQTEDVATYY |
| | SSRTAYMQLNSLTSEDSAVYFCAIS | CQQYWSIPWTFGAGTKLELK (SEQ ID |
| | RKPYFDYWGQGTTLTVSS (SEQ ID | NO: 171) |
| | NO: 170) | |
| I23RB16 | QIQLVQSGPELKKPGETVKISCKASG | DIKMTQSPASLAVSLGQRATISCRASKSV |
| 9 | YTFTTVGMQWVQKMPGKGFKWIG | STSDYSYMHWYQQKPGQPPKLLIYLASN |
| | WINTHSGEPKYTDDFKGRFAFSLET | LESGVPARFSGSGSGTDFTLNIHPVEEEDA |
| | SASTAYLQISNLKSEDTATYFCARSS | ATYYCQHSRELPRTFGAGTKLELK (SEQ |
| | ALTGAFAYWGQGTLVTVSS (SEQ | ID NO: 173) |
| | ID NO: 172) | |
| I23RB173 | DVQLQESGPGLVKPSQSLSLTCSVT | DIKMTQSPAIMSASPGEKVTITCSASSSVS |
| | GYSITSGYYWNWIRQFPGNKLEWM | YMHWFQQKPGTSPKLWIYSTSNLASGVP |
| | GYISYDGGNNYNPSLKNRISITRDTS | ARFSGSGSGTSYSLTISRMEAEDAATYYC |
| | KNQFFLKLNSVTTEDTATYYCARD | QQRSSYPLTFGDGTKLEMK (SEQ ID |
| | AGLLPYYAMDYWGQGTTLTVSS | NO: 175) |
| | (SEQ ID NO: 174) | |
| I23RB177 | EVQLQQSGPELLKPGASVKISCKAS | NTQMTQSPSSMYASLGERVTITCKASQDI |
| | GYTITDYTMHWVKQSHGKSLEWIG | NSYLSWFQQKPGKSPKALIYRANRLVDG |
| | GINPNYGGTSYNEKFKDKATLTVD | VPSRFSGSGSGQDYSLTISSLEYEDMGIYY |
| | KSSSTAYMELRSLTSEDSAVFYCVR | CQQYDEFPRTFGAGTKLELK (SEQ ID |
| | GNIYFDYWGQGTTLTVSS (SEQ ID | NO: 177) |
| | NO: 176) | |
| I23RB180 | QVTLKESGPGILQPSQTLSLTCSFSG | DIQMTQSPASLSASVGETVTITCRASVNIH |
| | FSLSTYGMGVGWIRQPSGKGLEWL | NYLAWYQQKQGKSPQLLVYNAKTLADG |
| | ANIWWNDSKYHNAALKSRLTISKD | VPSRFSGSGSGTQYSLKINSLQPEDFGSYY |
| | TSKNQVFLKIASVDTADTATYYCGR | CQHFWTTPYMYTFGAGTKLELK (SEQ ID |
| | IDGYYECVDVWGTGTSVTVSS (SEQ | NO: 179) |
| | ID NO: 178) | |
| I23RB182 | EVQLQQSGPGLVKPSQSLSLTCSVT | DIVMTQSPAIMSASLGEKVTMSCRASSSV |
| | GYSITSGYFWNWLRQFPGSKLEWM | NYMYWYQQKSDASPKLWIYYTSNLAPG |
| | GFIDNDGSNDYNPSLKNRISITRDTS | VPARFSGSGSGNSYSLTISSMEGEDAATY |
| | KNQFFLRLNSVTTEDTATYYCARGR | YCQQFTNSPWTFGAGTKLELK (SEQ ID |
| | LGFFDSWGPGTTLTVSS (SEQ ID | NO: 181) |
| | NO: 180) | |
| I23RB188 | QIQLVQSGPELKKPGETVKISCKASG | DVQMIQSPSSLSASLGERVSLTCRASQEIS |
| | YTFTNYGMNWVKQAPGKGLKWM | GNLSWLQQKPDGTIKRLIYAASTLDSGVP |
| | GWINTYTGAPTYADDFKGRFAFSLE | KRFSGSRSGSDYSLTISSLESEDFADYYCL |
| | TSASTAYLQINNFKNEDTATYFCAR | QYASYPWTFGAGTRLEIK (SEQ ID |
| | GNGYPYYYGINYWGQGTSVTVSS | NO: 183) |
| | (SEQ ID NO: 182) | |
| I23RB189 | DVQLQESGPDLVKPSQSLSLTCTVT | DIVLTQSPKSMSMSVGERVTLSCKASENV |
| | GYSITSGYNWHWIRQFPGNKLEWM | GTYVSWYQQKPEQSPKLLMYGASNRYT |
| | GYIHSSGTTNYSPSLRSRLSITRDTSK | GVPDRFTGSGSATDFTLTISSVQAEDLAD |
| | NQFFLQLNSVTTEDTATYYCTRQLG | YHCGQSYSYPPTFGGGTKLEIK (SEQ ID |
| | LKAMEYWGQGTTLTVSS (SEQ ID | NO: 185) |
| | NO: 184) | |
| I23RB27 | QVQLKESGPDLVQPSQTLSLTCTVS | DIQMTQSPTSLAVSLGQRATISCRASESVE |
| | GFSLTSYGVHWVRQPPGKGLEWVG | YYGTSLMHWYQQKPGQPPKLLIYAASNV |
| | TLGWDDKKSYNSALKSRLSISRDTS | ESGVPARFSGSGSGTDFSLNIHPVEEEDIA |
| | KSQVFLKLSSLQTEDTAMYYCTKE | MYFCQQGKKVPWTFGAGTKLEMK (SEQ |
| | DVSYAMDFWGQGTTLTVSS (SEQ | ID NO: 187) |
| | ID NO: 186) | |
| I23RB30 | QVTLKESGPGILQPSQTLSLTCSFSG | DIVLTQSQRFMSTSVGDRVSITCKASQNIR |
| | FSLSTSGMGVSWIRQPSGKGLEWLA | TAVAWYQQKPGQSPKALIYLPSNRHTGV |
| | HIYWDDDKRYNPSLKSRLTISKDTS | PDRFTGSGSGTDFTLTISNVQSEDLADYF |
| | SNQIFLKITSVDTADTATYYCARRTL | CLQHWNYPLTFGAGTKLELK (SEQ ID |
| | GGPPFDYWGQGTTLTVSS (SEQ ID | NO: 189) |
| | NO: 188) | |
| I23RB31 | EFQLQQSGAEMVRPGASVKLSCKA | DIVLTQSPATLSVTPGDRVSLSCRASQSIS |
| | SGYTFTGYWMNWVKQRPGQGLEW | DHLHWYQQKSHESPRLLIKFASQSISGIPS |
| | IGKIDPSDTETHYNQNFKDKATLTV | RFSGSGSGSDFTLSIDSVEPEDVGVYYCQ |
| | DKSSSTAYMQLNSLTSEDSAVYYC | NGHSFPLTFGSGTKLEIK (SEQ ID NO: 191) |
| | ARWGLGRNFAMDYWGQGTTLTVS | |
| | S (SEQ ID NO: 190) | |
| I23RB32 | EFQLQQSGPELVKPGASVKLSCKAS | DIVMTQSPSSLTVTAGEKVTMSCKSSQSL |
| | GYTFTDYALHWVKQSPGQGLEWIG | LNSGNQKNYLTWYQQKPGQPPKLLIYW |
| | WIYPGGGNTYYNDKFKGKATMTA | ASTRESGVPDRFTGSGSGTDFTLTISSVQA |
| | DKSSSTAYMQLSSLTSEDSAVYFCA | EDLAVYYCQNDYTYPLTFGSGTKLEIK |
| | RGRLRRDYPMDYWGQGTSVTVSS | (SEQ ID NO: 193) |
| | (SEQ ID NO: 192) | |
| I23RB33 | EVQLQQSGAELVKPGASVKLSCTAS | DIVLTQSPAIMSASPGEKVTMTCRASSSV |
| | GFNIRDIYIHWVKQRPEQGLEWIGRI | RSSYLHWYQQKSGASPKLWIYSTSNLAS |
| | DPANGHSIYAAKFQGKATITADTSL | GVPARFSGSGSGTSYSLTITSVEAEDAAT |
| | NIAYMQLSSLTSGDTAVYYCARGS | YYCQQYTGNPIFTFGSGTKLELK (SEQ ID |
| | DNDYGRVLAYWGLGTSVTVSS | NO: 195) |
| | (SEQ ID NO: 194) | |
| I23RB35 | DVQLQESGPGLVKPSQSLSLTCSVT | DIVLTQSPASLAVSLGQRATISCRASESVD |
| | GYSITSGHNWNWIRQFPGNKLEWL | SYGNSFMHWYQQKPGQPPKLLIYRASNL |
| | GYISYDGSNNYDPSLKNRISITRDTS | ESEIPARFSGRGSGTDFTLTINPVEADDVA |
| | KNQFFLKLNSVTTEDTATYYCARG | TYYCQQSNADPYTFGGGTKLEIK (SEQ ID |
| | GGYFGNSYRVFASWGQGTTVTVSS | NO: 197) |
| | (SEQ ID NO: 196) | |
| I23RB36 | QIQLVQSGAELVKPGASVKLSCTTS | DIVMTQSPKSMSMSVGERVTLSCKASEN |
| | GFNIKDTYIHWMKQRPEQGLEWIG | VGTSVSWFQQKPEQSPKLLIYGASNRYTG |
| | RIDPANGNTIYASKFQGKATLTTDT | VPDRFTGSGSATDFTLTISSVQAEDLADY |
| | SSNTAYMLLSSLTSGDTAVYYCTRG | HCGQSYSYPRTFGGGTKLEIK (SEQ ID |
| | TSWFAYWGQGTTVTVSS (SEQ ID | NO: 199) |
| | NO: 198) | |
| I23RB39 | EVQLQQSGPELVKPGAPVKISCKAS | DIQMTQSHKFMSTSVGDRVSITCKASQD |
| | GYTFTNYYIHWVKQRPGQGLEWIG | VSTAVAWYQQKPGQSPKLLIYSAFYRYT |
| | YIYPGNGGTAYNQKFKDKATLTAD | GVPDRFTGSGSGTDFTFTISSVQAEDLAV |
| | NPSSTAYMQLSSLTSEDSAVYFCAR | YYCQQHYSSPRTFGAGTKLELK (SEQ ID |
| | SRGGSRNYFDYWGQGTTLTVSS | NO: 201) |
| | (SEQ ID NO: 200) | |
| I23RB42 | EVQLQQSGAELVKPGASVKISCKAS | DIVLTQTAPSVPVTPGESVSISCRSSKSLL |
| | GYSFTDYIMNWVKQSHGKSLEWIG | HSNGNTYLYWFLQRPGQSPQLLIYRMSN |
| | NINPYFGFTSYNQEFKGKATLTVDK | LASGVPDRFSGSGSGTAFTLRISRVEAED |
| | SSSTAYMLLNSLTSEDSAVYYCSRG | VGVYYCMQHLEYPYTFGSGTRLEIK (SEQ |
| | SYYGYETFFDYWGQGTTLTVSS | ID NO: 203) |
| | (SEQ ID NO: 202) | |
| I23RB45 | QVTLKESGPGILQPSQTLSLTCSFSG | DIVLTQSQKFISTSVGDRVSITCKASQNVR |
| | FSLSTYGMGVSWIRQPSGKGLEWL | TAVAWYQQKPGQSPKLLIYLPSNRHTGV |
| | AHIYWDDDKRYNPSLKSRLTISKDT | PDRFTGSGSGTVFTLTISNVQSEDQADYF |
| | SNNQVFLKITSVDTADTATYYCAQR | CLQHWNYPLTFGAGTKLELK (SEQ ID |
| | ALPLLGQKAPFDYWGQGTTLTVSS | NO: 205) |
| | (SEQ ID NO: 204) | |
| I23RB47 | QVQLKESGPGLVAPSQSLSITCTVSG | DIVLTQSPASQSASLGESVTITCLASQTIGS |
| | FSLTNYDIHWVRQSPGKGLEWLGVI | WLAWYQQKPGKSPQLLIYAATRLADGVP |
| | WSDGRTDYKGAFISRLSISKDNSKS | SRFSGSGSGTKFSFKISSLQAEDFVFYYCH |
| | QVFFKMNSLQADDTAIYYCARNNY | QLYSIPLTFGGGTKLEMK (SEQ ID |
| | YRYGRNTMDYWGQGTSVTVSS | NO: 207) |
| | (SEQ ID NO: 206) | |
| I23RB51 | QVQLQQSGPDLVKPSQSLSLTCTVT | DIVLTQSPASLSASVGETVTITCRASGNIH |
| | GYSITSGYNWHWIRQFPGNKLEWM | NYLAWYQHKQGKSPQLLVYNAKTLADG |
| | GYVHYRDSTHYNPSLKSRISITRDTS | VPSRFSGSGSGTQYSLKINSLQPEDFGSYY |
| | KNQFFLQLNSVTTEDTATYYCARQL | CQYFWTTRTFGAGTKLELK (SEQ ID |
| | GLKAMDYWGQGTSVTVSS (SEQ ID | NO: 209) |
| | NO: 208) | |
| I23RB54 | EVQLQQSGGGLVKPGGSLKLSCAA | DIQMTQSPKFLLVSAGDRVTITCKASQSV |
| | SGFTFSSYAMSWVRQTPEKRLEWV | SNDVAWYQQKPGQSPRLLIYYASNRYTG |
| | AGISSGGSYIYYPDNVKGRFTISRDN | VPDRFTGSGYGTDFTFTISTVQAEDLAVY |
| | ARNTLYLQMSSLRSEDTAMYYCAR | FCQQDYSSLPTFGAGTKLEMK (SEQ ID |
| | KKSDYGAMDYWGQGTTLTVSS | NO: 211) |
| | (SEQ ID NO: 210) | |
| I23RB56 | EVKLVESGGGLVKPGGSLKLSCAAS | DIVMTQSPKFLLVSAGDRVTITCKASQSV |
| | GFTFSSYAMSWVRQTPEKRLEWVA | SNDVAWYQQKPGQSPKLLIYYASNRYTG |
| | AINSNDGSTYYPDTVKDRFTISRDN | VPDRFTGSGYGTVFTFTISTVQAEDLAVY |
| | AKNTLYLQMSSLRSEDTALYYCAR | FCQQDYSSPFTFGSGTKLEIK (SEQ ID |
| | HFLYFSNFYYFDYWGQGTTLTVSS | NO: 213) |
| | (SEQ ID NO: 212) | |
| I23RB57 | EFQLQQSGPELVKPGASVKMSCKAS | DIVMTQSPAIMSASLGERVTMTCTASSSV |
| | GYTFTNYYMHWVKQSHGKTIEWIG | SSSYLHWYQQKPGSSPKLWIYSTSNLASG |
| | YIYPNSGDTSYNQKFKGKATLTVDK | VPARFSGSGSGTSYSLTISSMEAEDAATY |
| | SSSTAYMELRSLTSEDSAVYNCARF | YCHQYHRSPFTFGSGTKLEIK (SEQ ID |
| | GGFYDAMDYWGQGTSVTVSS (SEQ | NO: 215) |
| | ID NO: 214) | |
| I23RB58 | EVKLVESGGGLVKPGGSLKLSCAAS | DIVMTQTPASLAVSLGQRATISCRASESV |
| | GFSFIIYTMSWVRQTPEKRLEWVAY | DSYGNSFMHWYQQKPGQPPKLLIYRASN |
| | ITSGGGITYYPDSVKGRFTISRDNAR | LESGIPARFSGSGSRTDFTLTINPVEADDV |
| | NTLYLQMSSLRSEDTAMYYCARHE | ATYYCQQSNEDPLTFGAGTKLEMK (SEQ |
| | GDYYDDYYRIGYAMDYWGQGTSV | ID NO: 217) |
| | TVSS (SEQ ID NO: 216) | |
| I23RB60 | DVQLQESGPELVKPGDSVKMSCEA | DIVMTQSTSSLSASLGDRVTISCRASQDIR |
| | SGYTFTDYYIDWVKQSHGKSLEWI | NNLNWYQQKPDGTVKLLIFYSSRLHSGV |
| | GYIYPNNGGISFNQKFEGKATLTVD | PSRFSGSGSGTDYSLTISNLEQEDIATYFC |
| | KSSSTAYMELHSLTSEDSAVYYCAR | QQGHTLPWTFGAGTKLELK (SEQ ID |
| | EKLRGRDYWGQGTTLTVSS (SEQ | NO: 219) |
| | ID NO: 218) | |
| I23RB61 | DVQLQESGAELVKPGASVKLSCKSF | DIQMTQSHKFLSTSVGDRITITCKASQDVI |
| | DYTFTSFDINWMKQRPGQGLEWIG | TAVAWYQQKSGQSPKLLIYWASTRHTGV |
| | WIFPGSGTTNYNEKFKGKATLTADK | PDRFTGSGSGTDYTLTISNVQAEDLALYY |
| | SSSTAYMHLTSLTSEDSAVYFCARR | CQQHYTTPPTFGAGTKLELK (SEQ ID |
| | LTGTRYFDVWGTGTTLTVSS (SEQ | NO: 221) |
| | ID NO: 220) | |
| I23RB65 | EVQLQQSGPELVKPGASVKISCKAS | QIVLTQSPASLAVSLGQRATISCRASQSVS |
| | GYTFTDHTMDWVKQSHGKSLEWIG | TSSYNYIHWYQQKPGQPPKLLIKYASNLE |
| | YIYPKSGNAAYSQKFKSEATLTVDK | SGVPARFSGSGSGTDFTLNIHPVEEDDTA |
| | SSSTAYMEFHSLTSEDSAVYYCAR | TYYCQHTWEIPYTFGSGTKLEIK (SEQ ID |
| | WGLRRDGFTYWGQGTSVTVSS | NO: 223) |
| | (SEQ ID NO: 222) | |
| I23RB66 | QVTLKESGPGILQPSQTLSLTCSFSG | DIVMTQSHKFMSTSVGDRVSITCKASQD |
| | FSLSTSNMGIGWIRQPSGKGLEWLA | VSTAVAWYQQKPGQSPKLLIYSASYRYT |
| | HIWWNSDKYYNPSLKSRLTISKDTS | GVPDRFTGSGSGTDFTFTFSSVQAEDLAV |
| | NNQVFLTITSVDTADTATYYCAQIR | YYCQQHYSTPYTFGGGTKLEIK (SEQ ID |
| | WLRFYFDYWGQGTTVTVSS (SEQ | NO: 225) |
| | ID NO: 224) | |
| I23RB67 | EVQLQQSGAELVKPGASVKLSCKA | DVVMTQSHKFMSTSVGDRVSITCKASQD |
| | SGYTFTSYWINWVKQRPGQGLEWI | VSTAVAWYQQKPGQSPKLLIYSAFYRYT |
| | GNIYPGSSSTNYNEKFKSKATLTVD | GVPDRFTGSGSGTDFTFTISSVQAEDLAV |
| | TSSSTAYMQLSSLTSDDSAVYYCAR | YYCQQHYSTPYTFGGGTKLELK (SEQ ID |
| | SAYGNRLAMDYWGQGTTLTVSS | NO: 227) |
| | (SEQ ID NO: 226) | |
| I23RB69 | EVQLQQSGAELVKPGASVKISCKAS | DIVMTQSHKFMSTSVGDRVSITCKASQD |
| | GYSFTGYNMNWVKQSHGKSLEWIG | VSTAVAWYQQKPGQSPKLLIYSASYRYT |
| | NINPFYDKTSYNQKFKGKATLTVDK | GVPDRFTGSGSGTDFTFTISSVQAEDLAV |
| | ASSTAYMQLNSLTSEDSAVYYCAR | YYCQQHYSTPWTFGGGTKLEMK (SEQ ID |
| | QLGLKGFAYWGQGTLVTVSA (SEQ | NO: 229) |
| | ID NO: 228) | |
| I23RB71 | DVQLQESGPDLVKPSQSLSLTCTVT | DIVMTQSHKFMSTSVGDRVSITCKASQD |
| | DYSITRGYNWHWIRQFPGNKLEWM | VITAVAWYQQKPGQSPKLLIYSASYRYT |
| | GYIHYSGSTNYNPSLKSRISITRDTS | GVPDRFTGSGSGTDFTFTISSVQAEDLAV |
| | KNQFFLQLNSVTTEDTATYYCARQ | YFCQQHFGTPYTFGSGTKLEMK (SEQ ID |
| | YGLKAMDYWGQGTSVTVSS (SEQ | NO: 231) |
| | ID NO: 230) | |
| I23RB73 | EVQLQQSGAELVKPGASVKISCKAS | DIVMTQSHKFMSTSVGDRVSITCKASQD |
| | GYTFTDHAIHWVKQRPEQGLEWIG | VSTAVAWYQQKPGQSPKLLIYWASTRLT |
| | YISLGNGDIKYSEKFRGKATLTADK | GVPDRFTGSGSGTDYTLTISSVQAEDLAL |
| | TSNTAYMQLNSLTSEDSAVYFCKRS | YYCQQYYNTPLTFGAGTKLELK (SEQ ID |
| | RVLRWTTYWYFDVWGAGTSVTVS | NO: 233) |
| | S (SEQ ID NO: 232) | |
| I23RB74 | DVQLQESGPGLVKPSQSLSLTCSVT | DIVLTQSPASLAVSLGQRATISCRASKSVS |
| | GYSITSGYYWNWIRQFPGNKLEWM | TSGSNFLHWYQQKPGQPPKLLIYFASNLE |
| | GYISYDGSFNYNPSLKNRLSITRDTS | SGVPARFSGSGSGTDFTLNIHPVEEEDAA |
| | MNQFFLKLNSVTSEDTATYYCARA | TYYCQHSRELPHTFGAGTKLELK (SEQ ID |
| | EIYYDYDGDFDYWGQGTTLTVSS | NO: 235) |
| | (SEQ ID NO: 234) | |
| I23RB76 | EVQLQQSGPELVKPGASVKMSCKA | DIVLTQSPATLCVTPGETVSLSCRASQNIY |
| | SGYTFTDYYMNWMKQSHGKSLEW | KNLHWFQQKSHRSPRLLIKYASDSISGIPS |
| | IGYISPKNGRTGYNQKFKGKATLTV | RFTGSGSGTDYTLSINSVKPEDEGIYYCLQ |
| | DKSSTTAYMELRSLTSEDSAVYYCA | GYSTPWTFGGGTKLEIK (SEQ ID NO: 237) |
| | KSNGYYALDYWGQGTTVTVSS | |
| | (SEQ ID NO: 236) | |
| I23RB77 | DVQLQESGPGLVAPSQSLSITCTVSG | DIVMTQSPASLALSLGQRATISCRASESV |
| | FSLSRYSVHWVRQPPGKGLEWLGM | DSYGNSFMHWYQQKPGQPPKLLIYLASN |
| | IWGSGSTDYNSALKSRLSINKDNSK | LESGVPARFSGSGSRTDFTLTIDPVEADD |
| | SQVFLKMNSLQTDDTAMYYCARV | AAIYYCQQNNEEPNTFGAGTKLEMK |
| | YYGGSPYAMDYWGQGTTVTVSS | (SEQ ID NO: 239) |
| | (SEQ ID NO: 238) | |
| I23RB78 | EFQLQQSGGGLVKPGGSLKLSCAAS | DIVMTQSPASLAVSLGQRATISCRASESV |
| | GFTFSNYTMSWVRQTPEKRLKWVA | DSYGISFMHWYQQKPGQPPKLLIYRASNL |
| | CISSGGGNTYYPDSVKGRFTISRDN | ESGIPARFSGSGSRTDFTLTINPVEADDVA |
| | ARNTLYLQMSSLRSEDTAMYYCAR | TYYCQQSNEDPWTFGAGTGLEIK (SEQ ID |
| | RNYRRAMDYWGQGTSVTVSS (SEQ | NO: 241) |
| | ID NO: 240) | |
| I23RB79 | DVQLQESGPELVRPGASVKMSCKA | DIQMTQSPKFLHVSAGDRVTITCKASQSV |
| | SGYTFTDYYMNWVKQSHGKSLEWI | INDVAWYQQKPGQSPKLLIYYASNRYTG |
| | GYIYPNNGDTVYNQKFKGKATLTV | VPDRFTGSGYGTDFTFTISTVQAEDLAIYF |
| | DKSSNTAYMELRSLTSEDSAVYYC | CQQDYSSPPTFGAGTRLEIK (SEQ ID |
| | ARGTLTGMYYFDYWGQGTSVTVSS | NO: 243) |
| | (SEQ ID NO: 242) | |
| I23RB80 | DVQLQESGPGLVQPSQSLSITCTVSG | DIVMTQSQKFMSTSEGDRVNITCKASQNI |
| | FSLTSYGVHWVRQSPGKGLEWLGV | RTAVAWYQQKPGQSPKPLIYLASNRHTG |
| | IWSGVSTDYNAAFISRLSISKDNSKS | VPDRFTGSGSGTDFTLTISNVQSEDLAEYF |
| | QVFFKMNSLQADDTAIYYCARSRG | CLQHWNYPLTFGAGTKLELK (SEQ ID |
| | SYYFDYWGQGTTLTVSS (SEQ ID | NO: 245) |
| | NO: 244) | |
| I23RB81 | DVQLQESGPGLVAPSQSLSITCTVSG | DIVMTQSHKFMSTSVGDRVSIICKASQDV |
| | FSLTSYAINWVRQPPGKGLEWLGVI | STAVAWYQQKPGQSPKLLIYSASYRYTG |
| | WTGGGTNYNSALKSRLSISKDNSKS | VPDRFTGSGSGTDFTFTISSVQAEDLAVY |
| | QLFLKMNSLQTDDTARYYCARNRD | YCQQHYSTPLTFGAGTRLEIK (SEQ ID |
| | YSNYRWYFDVWGAGTSVTVSS | NO: 247) |
| | (SEQ ID NO: 246) | |
| I23RB82 | QVQLKESGPGLVAPSQSLSITCTVSG | QIVLSQSHKFMSTSVGDRVSITCKASQDV |
| | FSLTNYALHWFRQSPGKGLEWLGVI | STAVAWYQQKPGQSPKLLIYSAFYRYTG |
| | WSDGSTDYNSPFISRLSISKDNSKSQ | VPDRFTGSGSGTDFTFTISSVQAEDLAVY |
| | VFFKMNSLQADDTAIYYCARSRSN | YCQQHYSTPYTFGGGTKLEIK (SEQ ID |
| | YGRWYFDVWGAGTTLTVSS (SEQ | NO: 249) |
| | ID NO: 248) | |
| I23RB83 | DVQLQESGPGLVAPSQSLSITCTVSG | DIQMTQSPASQSASLGESVTITCLASQTIG |
| | FSLTNYDVHWVRQSPGKGLEWLGV | SWLAWYQQKPGKSPQLLIYAATSLADGV |
| | IWSDGSTDYNAAFISRLSFSKDNSKR | PSRFSGSGSGTKFSFKISSLQAEDFVSYYC |
| | QVFFKMNSLQPDDTAIYYCARNNY | QQLYSSPLTFGGGTKLEIK (SEQ ID |
| | YRYGRNPMDYWGQGTTVTVSS | NO: 251) |
| | (SEQ ID NO: 250) | |
| I23RB85 | DVQLQESGPGLVAPSQSLSITCTVSG | DIVLTQSPLSLFVSLGDQASISCRSSQSLV |
| | FSFNTYAITWVRQPPGRGLEWLGVL | HSNGNTYLHWYLQKPGQSPKLLIYKVSN |
| | WPGGGISYNLALKSRLTISRDSSKSQ | RFSGVPDRFSGSGSGTDFTLKISRVEAEDL |
| | VFLEMNSLQTDDTARYYCARNWR | GVYFCSQSTHVPWTFGGGTRLEIK (SEQ |
| | HYDGSYAMDFWGQGTSVTVSS | ID NO: 253) |
| | (SEQ ID NO: 252) | |
| I23RB86 | EVQLQQSGAELVKPGASVKLSCKA | DIVMTQSPSSMSVSLGDTVSITCHASQGIS |
| | SGYTFTSYWLHWVKQRPGQGLEWI | SNIGWLQQKPGKSFKGLIYHGTNLEDEVP |
| | GEIDPSDSYTYYNQKFKGKATLTVD | SRFSGSGSGADYSLTIGSLESEDFADYYC |
| | KSSSTAYMQLSSLTSEDSAVYYCAR | VQYAQFPWTFGAGTKLEMK (SEQ ID |
| | SPYDYDWGAYWGQGTSVTVSS | NO: 255) |
| | (SEQ ID NO: 254) | |
| I23RB87 | EFQLQQSGPELVKPGTSVKMSCKAS | DIVMTQTTSSLSASLGDRVTISCRASQDIS |
| | GYTFTNYVMHWVKQKPGQGLEWI | NYLNWYQQKPDGTVKLLIYYTSRLHSGV |
| | GYINPYDASPKYNEKFKGKAILTSD | PSRFSGSGSGTDFSLTISNLEQEDIATYFC |
| | KSSSTAYMELSSLTSEDSAVYYCTR | QQGITLPWTFGAGTKLELK (SEQ ID |
| | DAKLDYFDYWGQGTTLTVSS (SEQ | NO: 257) |
| | ID NO: 256) | |
| I23RB91 | EFQLQQFGPELLKPGASVKISCKAS | DIKMTQSPSSMYASLGERVTITCKASQDI |
| | GYTFTDYIMHWVKQSHGKSLEWIG | NSYLSWCQQKPGKSPKALIYRANSLVDG |
| | GISPNYGGTGYNGKFKDKATLTVD | VPSRFSGSGSGQDYSLTISSLEYEDMGIYY |
| | KSSSTAYMELRSLTSEDSAVYYCAR | CLQYDEFPYTFGGGTKLEMK (SEQ ID |
| | FNNYYYFDYWGQGTTLTVSS (SEQ | NO: 259) |
| | ID NO: 258) | |
| I23RB92 | EVQLQQSGPEVVKPGASVKISCKAS | DIKMTQSPASLAVSLGQRATISCRASQSV |
| | GYTFTDHTMDWVKQSHGKNLEWI | STSKYDYIHWYQQKSGQPPRLLIKYASNL |
| | GYIYPKSGGTGYNQKFKNKATLTV | ESGVPARFSGSGSGTDFTLNIHPVEEEDTA |
| | DKSSSTAYMEFHSLTSDDSAVYYCA | TYYCQHTWEIPYTFGSGTKLELK (SEQ ID |
| | RWGLRRDGFVYWGQGTLVTVSA | NO: 261) |
| | (SEQ ID NO: 260) | |
| I23RB93 | QVQLKESGPGLVQPSQSLSITCTVSG | DIKMTQSPATLSVTPGDSVSLFCRASQSIS |
| | FSLTSYGVHWVRQSPGKGLEWLGV | NNLHWYQQKSHESPRLLIKYASQSISGIPS |
| | IWSGVSTDYNAAFISRLSISKDNSKS | RFSGSGSGTDFTLSINSVETEDFGMYFCQ |
| | QVFFKMNSLQADDTAIYYCARSRG | QSNSWPLTFGGGTKLEIK (SEQ ID |
| | SYYFDYWGQGTTLTVSS (SEQ ID | NO: 263) |
| | NO: 262) | |
| I23RB94 | DVQLQESGPELKKPGETVKISCKAS | DIVMTQSHKFMSTSVGDRVSITCKASQD |
| | GYTFTNYGMNWVRQAPGKSLKWM | VNTAVAWYQQKPGQSPKLLIYSAFYRYP |
| | GWINTNTGEPAYAEEFKGRFAFSLE | GVPDRFTGSGSGTDFTFTINSVQAEDLAV |
| | TSASTAYLQINNLKNEDMATYFCAR | YCCQQHYTSPLTFGDGTRLEIK (SEQ ID |
| | VRQTARATRGYFDVWGTGTTVTVS | NO: 265) |
| | S (SEQ ID NO: 264) | |
| I23RB95 | EVKLVESGGGLVQPGSSMKLSCTAS | DIVLTQSHKFMSTSVGDRVSITCKASQDV |
| | GFTFSDYYMAWVRQVPEKGLEWV | GTAVAWYQQKPGQSPKLLIYWASTRHTG |
| | ANINYDGSNTYYLDSLKSRFIISRDN | VPDRFTGSGSGTDFTLTISNVQSEDLADY |
| | AKNILYLQMSSLKSEDTATYYCARE | FCQQYSTYPYTVGGGTKLEIK (SEQ ID |
| | GYDEAMDYWGQGTTLTVSS (SEQ | NO: 267) |
| | ID NO: 266) | |
| I23RB98 | EFQLQQSGPELVKPGASVKMSCKAS | DIVLTQSPAIMSASLGERVTMTCTASSSVS |
| | GYTFTNYYMHWVKQSHGKTIEWIG | SSYLHWYQQKPGSSPKLWIYSTSNLASG |
| | YIYPNSGDTSYNQKFKGKATLTVDK | VPARFSGSGSGTSYSLTISSMEAEDAATY |
| | SSSTAYMELRSLTSEDSAVYTCARF | YCHQYHRSPFTFGSGTKLEMK (SEQ ID |
| | GGFYDAMDYWGQGTTVTVSS (SEQ | NO: 269) |
| | ID NO: 268) | |

TABLE 6
CDR AA Sequences of Exemplary Immunization Based IL-23R Antibodies

| Name | HCDR1 | HCDR2 | HCDR3 | LCDR1 | LCDR2 | LCDR3 |
|---|---|---|---|---|---|---|
| I23RB101 | GGTFSSYA | INPNSGGT | ARGYRYG | QDVRTA | SAS (SEQ | QQHYSTP |
| | (SEQ ID | (SEQ ID | PLDGFDI | (SEQ ID | ID NO: 274) | YT (SEQ ID |
| | NO: 270) | NO: 271) | (SEQ ID | NO: 273) | | NO: 275) |
| | | | NO: 272) | | | |
| I23RB102 | GYTFTKY | IYPGSIST | ARGDFYA | ENIYSH | AST (SEQ | QHFWGTP |
| | W (SEQ ID | (SEQ ID | LDY (SEQ | (SEQ ID | ID NO: 280) | WT (SEQ |
| | NO: 276) | NO: 277) | ID NO: 278) | NO: 279) | | ID NO: 281) |
| I23RB104 | GFTFSSYA | ISTGGST | ARGPHYY | ENIYSN | AAT (SEQ | QHFWGTP |
| | (SEQ ID | (SEQ ID | GGSYPFAY | (SEQ ID | ID NO: 286) | PT (SEQ ID |
| | NO: 282) | NO: 283) | (SEQ ID | NO: 285) | | NO: 287) |
| | | | NO: 284) | | | |
| I23RB144 | GYTFTSYI | IDPYNDDT | ARLGSINY | QSLLDSDG | LVS (SEQ | WQGIHFPL |
| | (SEQ ID | (SEQ ID | YDGSYFD | KTY (SEQ | ID NO: 292) | T (SEQ ID |
| | NO: 288) | NO: 289) | Y (SEQ ID | ID NO: 291) | | NO: 293) |
| | | | NO: 290) | | | |
| I23RB148 | GYSITSGY | IHSSGTT | TRQLGLK | QEVRTA | SAS (SEQ | QQHYTTP |
| | N (SEQ ID | (SEQ ID | AMEY | (SEQ ID | ID NO: 298) | YS (SEQ ID |
| | NO: 294) | NO: 295) | (SEQ ID | NO: 297) | | NO: 299) |
| | | | NO: 296) | | | |
| I23RB149 | GYSITSGY | IHFSDRT | SRQLGLK | QSLVHSN | KVS (SEQ | SQSTHVPL |
| | N (SEQ ID | (SEQ ID | ALDY (SEQ | GNTY (SEQ | ID NO: 304) | T (SEQ ID |
| | NO: 300) | NO: 301) | ID NO: 302) | ID NO: 303) | | NO: 305) |
| I23RB150 | AYTFTNY | IKTNTGEP | ARSRQLGL | QDVSTA | SAS (SEQ | QQHYSTP |
| | G (SEQ ID | (SEQ ID | RYYFDY | (SEQ ID | ID NO: 310) | YT (SEQ ID |
| | NO: 306) | NO: 307) | (SEQ ID | NO: 309) | | NO: 311) |
| | | | NO: 308) | | | |
| I23RB153 | GFTFSSDT | ISSNGGST | ASPGYPW | QDVGNA | WTS (SEQ | QQYNRYP |
| | (SEQ ID | (SEQ ID | FAY (SEQ | (SEQ ID | ID NO: 316) | LT (SEQ ID |
| | NO: 312) | NO: 313) | ID NO: 314) | NO: 315) | | NO: 317) |
| I23RB155 | GYSITSGH | ISYDGTN | ARGGGYY | ESVDTYG | RAS (SEQ | QQGNKDP |
| | S (SEQ ID | (SEQ ID | GGTYRVF | NSF (SEQ | ID NO: 322) | YT (SEQ ID |
| | NO: 318) | NO: 319) | AD (SEQ ID | ID NO: 321) | | NO: 323) |
| | | | NO: 320) | | | |
| I23RB156 | GFSLTSYA | IWTGGGT | ARNRDYS | QDVSTA | SAF (SEQ | QQHYSIPL |
| | (SEQ ID | (SEQ ID | NYRWYFD | (SEQ ID | ID NO: 328) | T (SEQ ID |
| | NO: 324) | NO: 325) | V (SEQ ID | NO: 327) | | NO: 329) |
| | | | NO: 326) | | | |
| I23RB157 | GFTFSRYT | ISTGGGDT | ARRTSYSY | ESVDNYGI | AAS (SEQ | QQSKEVP |
| | (SEQ ID | (SEQ ID | YNYVFDY | SF (SEQ ID | ID NO: 334) | WT (SEQ |
| | NO: 330) | NO: 331) | (SEQ ID | NO: 333) | | ID NO: 335) |
| | | | NO: 332) | | | |
| I23RB159 | GFTFSDYG | ISNLAYSV | ARHGDYR | QDISNY | YTS (SEQ | QQGNTLP |
| | (SEQ ID | (SEQ ID | NYGFAY | (SEQ ID | ID NO: 340) | WT (SEQ |
| | NO: 336) | NO: 337) | (SEQ ID | NO: 339) | | ID NO: 341) |
| | | | NO: 338) | | | |
| I23RB160 | GYSITSGY | IHYSSGT | ARQLGLK | QDVRTA | SAS (SEQ | QQHYTTP |
| | N (SEQ ID | (SEQ ID | AMDY | (SEQ ID | ID NO: 346) | YT (SEQ ID |
| | NO: 342) | NO: 343) | (SEQ ID | NO: 345) | | NO: 347) |
| | | | NO: 344) | | | |
| I23RB161 | VYAFSSS | IYPGDGDT | ARRYDFG | SSVSY | ATS (SEQ | QQWSSNP |
| | W (SEQ ID | (SEQ ID | SAMDY | (SEQ ID | ID NO: 352) | PT (SEQ ID |
| | NO: 348) | NO: 349) | (SEQ ID | NO: 351) | | NO: 353) |
| | | | NO: 350) | | | |
| I23RB162 | GYTFTNY | INPYNDGT | ARSRTGTR | QNVGTN | SAS (SEQ | QQYNSYPF |
| | V (SEQ ID | (SEQ ID | WYFDV | (SEQ ID | ID NO: 358) | T (SEQ ID |
| | NO: 354) | NO: 355) | (SEQ ID | NO: 357) | | NO: 359) |
| | | | NO: 356) | | | |
| I23RB165 | GFNIKDSL | IDPEDGKT | ARREVLD | KSVSTSGY | LAS (SEQ | QHSRELPL |
| | (SEQ ID | (SEQ ID | YFDY (SEQ | SY (SEQ ID | ID NO: 364) | T (SEQ ID |
| | NO: 360) | NO: 361) | ID NO: 362) | NO: 363) | | NO: 365) |
| I23RB166 | GYSITNGN | IRSSGST | ARERLRLR | QDVSTA | SAS (SEQ | QQHYSTPR |
| | HW (SEQ | (SEQ ID | WSYFDY | (SEQ ID | ID NO: 370) | T (SEQ ID |
| | ID NO: 366) | NO: 367) | (SEQ ID | NO: 369) | | NO: 371) |
| | | | NO: 368) | | | |
| I23RB168 | GYTFTDY | IYPAINNT | AISRKPYF | EDLYNR | GAT (SEQ | QQYWSIP |
| | Y (SEQ ID | (SEQ ID | DY (SEQ ID | (SEQ ID | ID NO: 376) | WT (SEQ |
| | NO: 372) | NO: 373) | NO: 374) | NO: 375) | | ID NO: 377) |
| I23RB169 | GYTFTTVG | INTHSGEP | ARSSALTG | KSVSTSDY | LAS (SEQ | QHSRELPR |
| | (SEQ ID | (SEQ ID | AFAY (SEQ | SY (SEQ ID | ID NO: 382) | T (SEQ ID |
| | NO: 378) | NO: 379) | ID NO: 380) | NO: 381) | | NO: 383) |
| I23RB173 | GYSITSGY | ISYDGGN | ARDAGLL | SSVSY | STS (SEQ | QQRSSYPL |
| | Y (SEQ ID | (SEQ ID | PYYAMDY | (SEQ ID | ID NO: 388) | T (SEQ ID |
| | NO: 384) | NO: 385) | (SEQ ID | NO: 387) | | NO: 389) |
| | | | NO: 386) | | | |
| I23RB177 | GYTITDYT | INPNYGGT | VRGNIYFD | QDINSY | RAN (SEQ | QQYDEFPR |
| | (SEQ ID | (SEQ ID | Y (SEQ ID | (SEQ ID | ID NO: 394) | T (SEQ ID |
| | NO: 390) | NO: 391) | NO: 392) | NO: 393) | | NO: 395) |
| I23RB180 | GFSLSTYG | IWWNDSK | GRIDGYYE | VNIHNY | NAK (SEQ | QHFWTTP |
| | MG (SEQ | (SEQ ID | CVDV | (SEQ ID | ID NO: 400) | YMYT |
| | ID NO: 396) | NO: 397) | (SEQ ID | NO: 399) | | (SEQ ID |
| | | | NO: 398) | | | NO: 401) |
| I23RB182 | GYSITSGY | IDNDGSN | ARGRLGFF | SSVNY | YTS (SEQ | QQFTNSP |
| | F (SEQ ID | (SEQ ID | DS (SEQ ID | (SEQ ID | ID NO: 406) | WT (SEQ |
| | NO: 402) | NO: 403) | NO: 404) | NO: 405) | | ID NO: 407) |
| I23RB188 | GYTFTNY | INTYTGAP | ARGNGYP | QEISGN | AAS (SEQ | LQYASYP |
| | G (SEQ ID | (SEQ ID | YYYGINY | (SEQ ID | ID NO: 412) | WT (SEQ |
| | NO: 408) | NO: 409) | (SEQ ID | NO: 411) | | ID NO: 413) |
| | | | NO: 410) | | | |
| I23RB189 | GYSITSGY | IHSSGTT | TRQLGLK | ENVGTY | GAS (SEQ | GQSYSYPP |
| | N (SEQ ID | (SEQ ID | AMEY | (SEQ ID | ID NO: 418) | T (SEQ ID |
| | NO: 414) | NO: 415) | (SEQ ID | NO: 417) | | NO: 419) |
| | | | NO: 416) | | | |
| I23RB27 | GFSLTSYG | LGWDDKK | TKEDVSY | ESVEYYGT | AAS (SEQ | QQGKKVP |
| | (SEQ ID | (SEQ ID | AMDF | SL (SEQ ID | ID NO: 424) | WT (SEQ |
| | NO: 420) | NO: 421) | (SEQ ID | NO: 423) | | ID NO: 425) |
| | | | NO: 422) | | | |
| I23RB30 | GFSLSTSG | IYWDDDK | ARRTLGGP | QNIRTA | LPS (SEQ | LQHWNYP |
| | MG (SEQ | (SEQ ID | PFDY (SEQ | (SEQ ID | ID NO: 430) | LT (SEQ ID |
| | ID NO: 426) | NO: 427) | ID NO: 428) | NO: 429) | | NO: 431) |
| I23RB31 | GYTFTGY | IDPSDTET | ARWGLGR | QSISDH | FAS (SEQ | QNGHSFPL |
| | W (SEQ ID | (SEQ ID | NFAMDY | (SEQ ID | ID NO: 436) | T (SEQ ID |
| | NO: 432) | NO: 433) | (SEQ ID | NO: 435) | | NO: 437) |
| | | | NO: 434) | | | |
| I23RB32 | GYTFTDY | IYPGGGNT | ARGRLRR | QSLLNSGN | WAS (SEQ | QNDYTYP |
| | A (SEQ ID | (SEQ ID | DYPMDY | QKNY | ID NO: 442) | LT (SEQ ID |
| | NO: 438) | NO: 439) | (SEQ ID | (SEQ ID | | NO: 443) |
| | | | NO: 440) | NO: 441) | | |
| I23RB33 | GFNIRDIY | IDPANGHS | ARGSDND | SSVRSSY | STS (SEQ | QQYTGNPI |
| | (SEQ ID | (SEQ ID | YGRVLAY | (SEQ ID | ID NO: 448) | FT (SEQ ID |
| | NO: 444) | NO: 445) | (SEQ ID | NO: 447) | | NO: 449) |
| | | | NO: 446) | | | |
| I23RB35 | GYSITSGH | ISYDGSN | ARGGGYF | ESVDSYG | RAS (SEQ | QQSNADP |
| | N (SEQ ID | (SEQ ID | GNSYRVF | NSF (SEQ | ID NO: 454) | YT (SEQ ID |
| | NO: 450) | NO: 451) | AS (SEQ ID | ID NO: 453) | | NO: 455) |
| | | | NO: 452) | | | |
| I23RB36 | GFNIKDTY | IDPANGNT | TRGTSWF | ENVGTS | GAS (SEQ | GQSYSYPR |
| | (SEQ ID | (SEQ ID | AY (SEQ ID | (SEQ ID | ID NO: 460) | T (SEQ ID |
| | NO: 456) | NO: 457) | NO: 458) | NO: 459) | | NO: 461) |
| I23RB39 | GYTFTNY | IYPGNGGT | ARSRGGSR | QDVSTA | SAF (SEQ | QQHYSSPR |
| | Y (SEQ ID | (SEQ ID | NYFDY | (SEQ ID | ID NO: 466) | T (SEQ ID |
| | NO: 462) | NO: 463) | (SEQ ID | NO: 465) | | NO: 467) |
| | | | NO: 464) | | | |
| I23RB42 | GYSFTDYI | INPYFGFT | SRGSYYG | KSLLHSNG | RMS (SEQ | MQHLEYP |
| | (SEQ ID | (SEQ ID | YETFFDY | NTY (SEQ | ID NO: 472) | YT (SEQ ID |
| | NO: 468) | NO: 469) | (SEQ ID | ID NO: 471) | | NO: 473) |
| | | | NO: 470) | | | |
| I23RB45 | GFSLSTYG | IYWDDDK | AQRALPLL | QNVRTA | LPS (SEQ | LQHWNYP |
| | MG (SEQ | (SEQ ID | GQKAPFD | (SEQ ID | ID NO: 478) | LT (SEQ ID |
| | ID NO: 474) | NO: 475) | Y (SEQ ID | NO: 477) | | NO: 479) |
| | | | NO: 476) | | | |
| I23RB47 | GFSLTNYD | IWSDGRT | ARNNYYR | QTIGSW | AAT (SEQ | HQLYSIPL |
| | (SEQ ID | (SEQ ID | YGRNTMD | (SEQ ID | ID NO: 484) | T (SEQ ID |
| | NO: 480) | NO: 481) | Y (SEQ ID | NO: 483) | | NO: 485) |
| | | | NO: 482) | | | |
| I23RB51 | GYSITSGY | VHYRDST | ARQLGLK | GNIHNY | NAK (SEQ | QYFWTTR |
| | N (SEQ ID | (SEQ ID | AMDY | (SEQ ID | ID NO: 490) | T (SEQ ID |
| | NO: 486) | NO: 487) | (SEQ ID | NO: 489) | | NO: 491) |
| | | | NO: 488) | | | |
| I23RB54 | GFTFSSYA | ISSGGSYI | ARKKSDY | QSVSND | YAS (SEQ | QQDYSSLP |
| | (SEQ ID | (SEQ ID | GAMDY | (SEQ ID | ID NO: 496) | T (SEQ ID |
| | NO: 492) | NO: 493) | (SEQ ID | NO: 495) | | NO: 497) |
| | | | NO: 494) | | | |
| I23RB56 | GFTFSSYA | INSNDGST | ARHFLYFS | QSVSND | YAS (SEQ | QQDYSSPF |
| | (SEQ ID | (SEQ ID | NFYYFDY | (SEQ ID | ID NO: 502) | T (SEQ ID |
| | NO: 498) | NO: 499) | (SEQ ID | NO: 501) | | NO: 503) |
| | | | NO: 500) | | | |
| I23RB57 | GYTFTNY | IYPNSGDT | ARFGGFY | SSVSSSY | STS (SEQ | HQYHRSPF |
| | Y (SEQ ID | (SEQ ID | DAMDY | (SEQ ID | ID NO: 508) | T (SEQ ID |
| | NO: 504) | NO: 505) | (SEQ ID | NO: 507) | | NO: 509) |
| | | | NO: 506) | | | |
| I23RB58 | GFSFIIYT | ITSGGGIT | ARHEGDY | ESVDSYG | RAS (SEQ | QQSNEDPL |
| | (SEQ ID | (SEQ ID | YDDYYRI | NSF (SEQ | ID NO: 514) | T (SEQ ID |
| | NO: 510) | NO: 511) | GYAMDY | ID NO: 513) | | NO: 515) |
| | | | (SEQ ID | | | |
| | | | NO: 512) | | | |
| I23RB60 | GYTFTDY | IYPNNGGI | AREKLRG | QDIRNN | YSS (SEQ | QQGHTLP |
| | Y (SEQ ID | (SEQ ID | RDY (SEQ | (SEQ ID | ID NO: 520) | WT (SEQ |
| | NO: 516) | NO: 517) | ID NO: 518) | NO: 519) | | ID NO: 521) |
| I23RB61 | DYTFTSFD | IFPGSGTT | ARRLTGTR | QDVITA | WAS (SEQ | QQHYTTPP |
| | (SEQ ID | (SEQ ID | YFDV (SEQ | (SEQ ID | ID NO: 526) | T (SEQ ID |
| | NO: 522) | NO: 523) | ID NO: 524) | NO: 525) | | NO: 527) |
| I23RB65 | GYTFTDHT | IYPKSGNA | ARWGLRR | QSVSTSSY | YAS (SEQ | QHTWEIPY |
| | (SEQ ID | (SEQ ID | DGFTY | NY (SEQ ID | ID NO: 532) | T (SEQ ID |
| | NO: 528) | NO: 529) | (SEQ ID | NO: 531) | | NO: 533) |
| | | | NO: 530) | | | |
| I23RB66 | GFSLSTSN | IWWNSDK | AQIRWLRF | QDVSTA | SAS (SEQ | QQHYSTP |
| | MG (SEQ | (SEQ ID | YFDY (SEQ | (SEQ ID | ID NO: 538) | YT (SEQ ID |
| | ID NO: 534) | NO: 535) | ID NO: 536) | NO: 537) | | NO: 539) |
| I23RB67 | GYTFTSY | IYPGSSST | ARSAYGN | QDVSTA | SAF (SEQ | QQHYSTP |
| | W (SEQ ID | (SEQ ID | RLAMDY | (SEQ ID | ID NO: 544) | YT (SEQ ID |
| | NO: 540) | NO: 541) | (SEQ ID | NO: 543) | | NO: 545) |
| | | | NO: 542) | | | |
| I23RB69 | GYSFTGY | INPFYDKT | ARQLGLK | QDVSTA | SAS (SEQ | QQHYSTP |
| | N (SEQ ID | (SEQ ID | GFAY (SEQ | (SEQ ID | ID NO: 550) | WT (SEQ |
| | NO: 546) | NO: 547) | ID NO: 548) | NO: 549) | | ID NO: 551) |
| I23RB71 | DYSITRGY | IHYSGST | ARQYGLK | QDVITA | SAS (SEQ | QQHFGTP |
| | N (SEQ ID | (SEQ ID | AMDY | (SEQ ID | ID NO: 556) | YT (SEQ ID |
| | NO: 552) | NO: 553) | (SEQ ID | NO: 555) | | NO: 557) |
| | | | NO: 554) | | | |
| I23RB73 | GYTFTDH | ISLGNGDI | KRSRVLR | QDVSTA | WAS (SEQ | QQYYNTP |
| | A (SEQ ID | (SEQ ID | WTTYWYF | (SEQ ID | ID NO: 562) | LT (SEQ ID |
| | NO: 558) | NO: 559) | DV (SEQ ID | NO: 561) | | NO: 563) |
| | | | NO: 560) | | | |
| I23RB74 | GYSITSGY | ISYDGSF | ARAEIYYD | KSVSTSGS | FAS (SEQ | QHSRELPH |
| | Y (SEQ ID | (SEQ ID | YDGDFDY | NF (SEQ ID | ID NO: 568) | T (SEQ ID |
| | NO: 564) | NO: 565) | (SEQ ID | NO: 567) | | NO: 569) |
| | | | NO: 566) | | | |
| I23RB76 | GYTFTDY | ISPKNGRT | AKSNGYY | QNIYKN | YAS (SEQ | LOGYSTP |
| | Y (SEQ ID | (SEQ ID | ALDY (SEQ | (SEQ ID | ID NO: 574) | WT (SEQ |
| | NO: 570) | NO: 571) | ID NO: 572) | NO: 573) | | ID NO: 575) |
| I23RB77 | GFSLSRYS | IWGSGST | ARVYYGG | ESVDSYG | LAS (SEQ | QQNNEEP |
| | (SEQ ID | (SEQ ID | SPYAMDY | NSF (SEQ | ID NO: 580) | NT (SEQ ID |
| | NO: 576) | NO: 577) | (SEQ ID | ID NO: 579) | | NO: 581) |
| | | | NO: 578) | | | |
| I23RB78 | GFTFSNYT | ISSGGGNT | ARRNYRR | ESVDSYGI | RAS (SEQ | QQSNEDP |
| | (SEQ ID | (SEQ ID | AMDY | SF (SEQ ID | ID NO: 586) | WT (SEQ |
| | NO: 582) | NO: 583) | (SEQ ID | NO: 585) | | ID NO: 587) |
| | | | NO: 584) | | | |
| I23RB79 | GYTFTDY | IYPNNGDT | ARGTLTG | QSVIND | YAS (SEQ | QQDYSSPP |
| | Y (SEQ ID | (SEQ ID | MYYFDY | (SEQ ID | ID NO: 592) | T (SEQ ID |
| | NO: 588) | NO: 589) | (SEQ ID | NO: 591) | | NO: 593) |
| | | | NO: 590) | | | |
| I23RB80 | GFSLTSYG | IWSGVST | ARSRGSY | QNIRTA | LAS (SEQ | LQHWNYP |
| | (SEQ ID | (SEQ ID | YFDY (SEQ | (SEQ ID | ID NO: 598) | LT (SEQ ID |
| | NO: 594) | NO: 595) | ID NO: 596) | NO: 597) | | NO: 599) |
| I23RB81 | GFSLTSYA | IWTGGGT | ARNRDYS | QDVSTA | SAS (SEQ | QQHYSTPL |
| | (SEQ ID | (SEQ ID | NYRWYFD | (SEQ ID | ID NO: 604) | T (SEQ ID |
| | NO: 600) | NO: 601) | V (SEQ ID | NO: 603) | | NO: 605) |
| | | | NO: 602) | | | |
| I23RB82 | GFSLTNYA | IWSDGST | ARSRSNY | QDVSTA | SAF (SEQ | QQHYSTP |
| | (SEQ ID | (SEQ ID | GRWYFDV | (SEQ ID | ID NO: 610) | YT (SEQ ID |
| | NO: 606) | NO: 607) | (SEQ ID | NO: 609) | | NO: 611) |
| | | | NO: 608) | | | |
| I23RB83 | GFSLTNYD | IWSDGST | ARNNYYR | QTIGSW | AAT (SEQ | QQLYSSPL |
| | (SEQ ID | (SEQ ID | YGRNPMD | (SEQ ID | ID NO: 616) | T (SEQ ID |
| | NO: 612) | NO: 613) | Y (SEQ ID | NO: 615) | | NO: 617) |
| | | | NO: 614) | | | |
| I23RB85 | GFSFNTYA | LWPGGGI | ARNWRHY | QSLVHSN | KVS (SEQ | SQSTHVP |
| | (SEQ ID | (SEQ ID | DGSYAMD | GNTY (SEQ | ID NO: 622) | WT (SEQ |
| | NO: 618) | NO: 619) | F (SEQ ID | ID NO: 621) | | ID NO: 623) |
| | | | NO: 620) | | | |
| I23RB86 | GYTFTSY | IDPSDSYT | ARSPYDY | QGISSN | HGT (SEQ | VQYAQFP |
| | W (SEQ ID | (SEQ ID | DWGAY | (SEQ ID | ID NO: 628) | WT (SEQ |
| | NO: 624) | NO: 625) | (SEQ ID | NO: 627) | | ID NO: 629) |
| | | | NO: 626) | | | |
| I23RB87 | GYTFTNY | INPYDASP | TRDAKLD | QDISNY | YTS (SEQ | QQGITLPW |
| | V (SEQ ID | (SEQ ID | YFDY (SEQ | (SEQ ID | ID NO: 634) | T (SEQ ID |
| | NO: 630) | NO: 631) | ID NO: 632) | NO: 633) | | NO: 635) |
| I23RB91 | GYTFTDYI | ISPNYGGT | ARFNNYY | QDINSY | RAN (SEQ | LQYDEFPY |
| | (SEQ ID | (SEQ ID | YFDY (SEQ | (SEQ ID | ID NO: 640) | T (SEQ ID |
| | NO: 636) | NO: 637) | ID NO: 638) | NO: 639) | | NO: 641) |
| I23RB92 | GYTFTDHT | IYPKSGGT | ARWGLRR | QSVSTSKY | YAS (SEQ | QHTWEIPY |
| | (SEQ ID | (SEQ ID | DGFVY | DY (SEQ ID | ID NO: 646) | T (SEQ ID |
| | NO: 642) | NO: 643) | (SEQ ID | NO: 645) | | NO: 647) |
| | | | NO: 644) | | | |
| I23RB93 | GFSLTSYG | IWSGVST | ARSRGSY | QSISNN | YAS (SEQ | QQSNSWP |
| | (SEQ ID | (SEQ ID | YFDY (SEQ | (SEQ ID | ID NO: 652) | LT (SEQ ID |
| | NO: 648) | NO: 649) | ID NO: 650) | NO: 651) | | NO: 653) |
| I23RB94 | GYTFTNY | INTNTGEP | ARVRQTA | QDVNTA | SAF (SEQ | QQHYTSPL |
| | G (SEQ ID | (SEQ ID | RATRGYF | (SEQ ID | ID NO: 658) | T (SEQ ID |
| | NO: 654) | NO: 655) | DV (SEQ ID | NO: 657) | | NO: 659) |
| | | | NO: 656) | | | |
| I23RB95 | GFTFSDYY | INYDGSNT | AREGYDE | QDVGTA | WAS (SEQ | QQYSTYP |
| | (SEQ ID | (SEQ ID | AMDY | (SEQ ID | ID NO: 664) | YT (SEQ ID |
| | NO: 660) | NO: 661) | (SEQ ID | NO: 663) | | NO: 665) |
| | | | NO: 662) | | | |
| I23RB98 | GYTFTNY | IYPNSGDT | ARFGGFY | SSVSSSY | STS (SEQ | HQYHRSPF |
| | Y (SEQ ID | (SEQ ID | DAMDY | (SEQ ID | ID NO: 670) | T (SEQ ID |
| | NO: 666) | NO: 667) | (SEQ ID | NO: 669) | | NO: 671) |
| | | | NO: 668) | | | |

Examples of phage display based IL-23R antibodies or antigen binding fragments of the present disclosure are summarized in Table 7, Table 8, and Table 9. All of the phage display based antibodies with the exception of 123RB332 and 123RB7 were derived from the human phage display and are human antibodies. Antibody 123RB332, corresponding to VH SEQ ID NO: 898 and VL SEQ ID NO:899 and also antibody 123RB7, corresponding to VH SEQ ID NO:798 and VL SEQ ID NO:799, are mouse antibodies.

TABLE 7
HC and LC AA Sequences of Exemplary Phage Display Based IL-23R antibodies

| Names | HC Sequence | LC Sequence |
|---|---|---|
| I23RB1 | QVQLVQSGAEVKKPGSSVKVSCKASG | EIVLTQSPATLSLSPGERATLSCRASQS |
| | GTFKNYAISWVRQAPGQGLEWMGWI | VSSYLAWYQQKPGQAPRLLIYDASNR |
| | RPIFGTAYYAQKFQGRVTITADESTSTA | ATGIPARFSGSGSGTDFTLTISSLEPEDF |
| | YMELSSLRSEDTAVYYCARDSGSRYA | AVYYCQQRSNWPLTFGQGTKVEIKRA |
| | GRYAFDYWGQGTLVTVSSAKTTAPSV | DAAPTVSIFPPSSEQLTSGGASVVCFLN |
| | YPLAPVCGDTTGSSVTLGCLVKGYFPE | NFYPKDINVKWKIDGSERQNGVLNSW |
| | PVTLTWNSGSLSSGVHTFPAVLQSDLY | TDQDSKDSTYSMSSTLTLTKDEYERHN |
| | TLSSSVTVTSSTWPSQSITCNVAHPASS | SYTCEATHKTSTSPIVKSFNRNEC (SEQ |
| | TKVDKKIEPRGPTIKPCPPCKCPAPNLL | ID NO: 673) |
| | GGPSVFIFPPKIKDVLMISLSPIVTCVVV | |
| | DVSEDDPDVQISWFVNNVEVHTAQTQ | |
| | THREDYNSTLRVVSALPIQHQDWMSG | |
| | KEFKCKVNNKDLPAPIERTISKPKGSVR | |
| | APQVYVLPPPEEEMTKKQVTLTCMVT | |
| | DFMPEDIYVEWTNNGKTELNYKNTEP | |
| | VLDSDGSYFMYSKLRVEKKNWVERNS | |
| | YSCSVVHEGLHNHHTTKSFSRTPGK | |
| | (SEQ ID NO: 672) | |
| I23RB2 | QVQLVQSGAEVKKPGSSVKVSCKASG | EIVLTQSPATLSLSPGERATLSCRASQS |
| | GTFSSYAISWVRQAPGQGLEWMGGIIP | VSSYLAWYQQKPGQAPRLLIYDASNR |
| | IFGTANYAQKFQGRVTITADESTSTAY | ATGIPARFSGSGSGTDFTLTISSLEPEDF |
| | MELSSLRSEDTAVYYCARDRYRYLGA | AVYYCQQRSNWPLTFGQGTKVEIKRA |
| | NGFDYWGQGTLVTVSSAKTTAPSVYP | DAAPTVSIFPPSSEQLTSGGASVVCFLN |
| | LAPVCGDTTGSSVTLGCLVKGYFPEPV | NFYPKDINVKWKIDGSERQNGVLNSW |
| | TLTWNSGSLSSGVHTFPAVLQSDLYTL | TDQDSKDSTYSMSSTLTLTKDEYERHN |
| | SSSVTVTSSTWPSQSITCNVAHPASSTK | SYTCEATHKTSTSPIVKSFNRNEC (SEQ |
| | VDKKIEPRGPTIKPCPPCKCPAPNLLGG | ID NO: 675) |
| | PSVFIFPPKIKDVLMISLSPIVTCVVVDV | |
| | SEDDPDVQISWFVNNVEVHTAQTQTH | |
| | REDYNSTLRVVSALPIQHQDWMSGKE | |
| | FKCKVNNKDLPAPIERTISKPKGSVRAP | |
| | QVYVLPPPEEEMTKKQVTLTCMVTDF | |
| | MPEDIYVEWTNNGKTELNYKNTEPVL | |
| | DSDGSYFMYSKLRVEKKNWVERNSYS | |
| | CSVVHEGLHNHHTTKSFSRTPGK (SEQ | |
| | ID NO: 674) | |
| I23RB3 | QVQLVQSGAEVKKPGSSVKVSCKASG | EIVLTQSPATLSLSPGERATLSCRASQS |
| | GTFKSYSIHWVRQAPGQGLEWMGGIIP | VSSYLAWYQQKPGQAPRLLIYDASNR |
| | YFGYANYAQKFQGRVTITADESTSTAY | ATGIPARFSGSGSGTDFTLTISSLEPEDF |
| | MELSSLRSEDTAVYYCARERGYYGSIY | AVYYCQQRSNWPLTFGQGTKVEIKRA |
| | YGGGFDYWGQGTLVTVSSAKTTAPSV | DAAPTVSIFPPSSEQLTSGGASVVCFLN |
| | YPLAPVCGDTTGSSVTLGCLVKGYFPE | NFYPKDINVKWKIDGSERQNGVLNSW |
| | PVTLTWNSGSLSSGVHTFPAVLQSDLY | TDQDSKDSTYSMSSTLTLTKDEYERHN |
| | TLSSSVTVTSSTWPSQSITCNVAHPASS | SYTCEATHKTSTSPIVKSFNRNEC (SEQ |
| | TKVDKKIEPRGPTIKPCPPCKCPAPNLL | ID NO: 677) |
| | GGPSVFIFPPKIKDVLMISLSPIVTCVVV | |
| | DVSEDDPDVQISWFVNNVEVHTAQTQ | |
| | THREDYNSTLRVVSALPIQHQDWMSG | |
| | KEFKCKVNNKDLPAPIERTISKPKGSVR | |
| | APQVYVLPPPEEEMTKKQVTLTCMVT | |
| | DFMPEDIYVEWTNNGKTELNYKNTEP | |
| | VLDSDGSYFMYSKLRVEKKNWVERNS | |
| | YSCSVVHEGLHNHHTTKSFSRTPGK | |
| | (SEQ ID NO: 676) | |
| I23RB4 | QVQLVQSGAEVKKPGSSVKVSCKASG | EIVLTQSPATLSLSPGERATLSCRASQS |
| | GTFKTYAIHWVRQAPGQGLEWMGWII | VSSYLAWYQQKPGQAPRLLIYDASNR |
| | PIFGTANYAQKFQGRVTITADESTSTA | ATGIPARFSGSGSGTDFTLTISSLEPEDF |
| | YMELSSLRSEDTAVYYCARDGGYYGS | AVYYCQQRSNWPLTFGQGTKVEIKRA |
| | RYYPRSYYFDYWGQGTLVTVSSAKTT | DAAPTVSIFPPSSEQLTSGGASVVCFLN |
| | APSVYPLAPVCGDTTGSSVTLGCLVKG | NFYPKDINVKWKIDGSERQNGVLNSW |
| | YFPEPVTLTWNSGSLSSGVHTFPAVLQ | TDQDSKDSTYSMSSTLTLTKDEYERHN |
| | SDLYTLSSSVTVTSSTWPSQSITCNVAH | SYTCEATHKTSTSPIVKSFNRNEC (SEQ |
| | PASSTKVDKKIEPRGPTIKPCPPCKCPA | ID NO: 679) |
| | PNLLGGPSVFIFPPKIKDVLMISLSPIVT | |
| | CVVVDVSEDDPDVQISWFVNNVEVHT | |
| | AQTQTHREDYNSTLRVVSALPIQHQD | |
| | WMSGKEFKCKVNNKDLPAPIERTISKP | |
| | KGSVRAPQVYVLPPPEEEMTKKQVTL | |
| | TCMVTDFMPEDIYVEWTNNGKTELNY | |
| | KNTEPVLDSDGSYFMYSKLRVEKKNW | |
| | VERNSYSCSVVHEGLHNHHTTKSFSRT | |
| | PGK (SEQ ID NO: 678) | |
| I23RB5 | QVQLVQSGAEVKKPGSSVKVSCKASG | DIQMTQSPSSLSASVGDRVTITCRASQS |
| | GTFSSYAISWVRQAPGQGLEWMGGIIP | ISSYLNWYQQKPGKAPKLLIYAASSLQ |
| | IFGTANYAQKFQGRVTITADESTSTAY | SGVPSRFSGSGSGTDFTLTISSLQPEDF |
| | MELSSLRSEDTAVYYCARDSYYSTVT | ATYYCQQSYSTPLTFGQGTKVEIKRAD |
| | YYYFDYWGQGTLVTVSSAKTTAPSVY | AAPTVSIFPPSSEQLTSGGASVVCFLNN |
| | PLAPVCGDTTGSSVTLGCLVKGYFPEP | FYPKDINVKWKIDGSERQNGVLNSWT |
| | VTLTWNSGSLSSGVHTFPAVLQSDLYT | DQDSKDSTYSMSSTLTLTKDEYERHNS |
| | LSSSVTVTSSTWPSQSITCNVAHPASST | YTCEATHKTSTSPIVKSFNRNEC (SEQ |
| | KVDKKIEPRGPTIKPCPPCKCPAPNLLG | ID NO: 681) |
| | GPSVFIFPPKIKDVLMISLSPIVTCVVVD | |
| | VSEDDPDVQISWFVNNVEVHTAQTQT | |
| | HREDYNSTLRVVSALPIQHQDWMSGK | |
| | EFKCKVNNKDLPAPIERTISKPKGSVRA | |
| | PQVYVLPPPEEEMTKKQVTLTCMVTD | |
| | FMPEDIYVEWTNNGKTELNYKNTEPV | |
| | LDSDGSYFMYSKLRVEKKNWVERNSY | |
| | SCSVVHEGLHNHHTTKSFSRTPGK | |
| | (SEQ ID NO: 680) | |
| I23RB6 | QVQLVQSGAEVKKPGSSVKVSCKASG | DIVMTQSPDSLAVSLGERATINCKSSQS |
| | GTFSSYAISWVRQAPGQGLEWMGGIIP | VLYSSNNKNYLAWYQQKPGQPPKLLI |
| | IFGTANYAQKFQGRVTITADESTSTAY | YWASTRESGVPDRFSGSGSGTDFTLTIS |
| | MELSSLRSEDTAVYYCARGRYIRSYDY | SLQAEDVAVYYCQQYYSTPLTFGQGT |
| | FDYWGQGTLVTVSSAKTTAPSVYPLA | KVEIKRADAAPTVSIFPPSSEQLTSGGA |
| | PVCGDTTGSSVTLGCLVKGYFPEPVTL | SVVCFLNNFYPKDINVKWKIDGSERQN |
| | TWNSGSLSSGVHTFPAVLQSDLYTLSS | GVLNSWTDQDSKDSTYSMSSTLTLTK |
| | SVTVTSSTWPSQSITCNVAHPASSTKV | DEYERHNSYTCEATHKTSTSPIVKSFN |
| | DKKIEPRGPTIKPCPPCKCPAPNLLGGP | RNEC (SEQ ID NO: 683) |
| | SVFIFPPKIKDVLMISLSPIVTCVVVDVS | |
| | EDDPDVQISWFVNNVEVHTAQTQTHR | |
| | EDYNSTLRVVSALPIQHQDWMSGKEF | |
| | KCKVNNKDLPAPIERTISKPKGSVRAP | |
| | QVYVLPPPEEEMTKKQVTLTCMVTDF | |
| | MPEDIYVEWTNNGKTELNYKNTEPVL | |
| | DSDGSYFMYSKLRVEKKNWVERNSYS | |
| | CSVVHEGLHNHHTTKSFSRTPGK (SEQ | |
| | ID NO: 682) | |
| I23RB7 | QIQLVQSGPELKKPGETVKISCKASGY | QIVLTQSPAIMSASPGEKVTMTCSASSS |
| | TFTTYGMSWVKQAPGKGLKWMGWIN | VSYMYWYQQKPGSSPRLLIYDTSNLA |
| | TYSGVPTYADDFKGRFAFSLETSASTA | SGVPVRFSGSGSGTSYSLTISRMEAED |
| | YLQINNLKNEDTATYYCARRDRYGYV | AATYYCQQWSSYPWTFGGGTKLEIKR |
| | AGAFAYWGQGTLVTVSSAKTTAPSVY | ADAAPTVSIFPPSSEQLTSGGASVVCFL |
| | PLAPVCGDTTGSSVTLGCLVKGYFPEP | NNFYPKDINVKWKIDGSERQNGVLNS |
| | VTLTWNSGSLSSGVHTFPAVLQSDLYT | WTDQDSKDSTYSMSSTLTLTKDEYER |
| | LSSSVTVTSSTWPSQSITCNVAHPASST | HNSYTCEATHKTSTSPIVKSFNRNEC |
| | KVDKKIEPRGPTIKPCPPCKCPAPNLLG | (SEQ ID NO: 685) |
| | GPSVFIFPPKIKDVLMISLSPIVTCVVVD | |
| | VSEDDPDVQISWFVNNVEVHTAQTQT | |
| | HREDYNSTLRVVSALPIQHQDWMSGK | |
| | EFKCKVNNKDLPAPIERTISKPKGSVRA | |
| | PQVYVLPPPEEEMTKKQVTLTCMVTD | |
| | FMPEDIYVEWTNNGKTELNYKNTEPV | |
| | LDSDGSYFMYSKLRVEKKNWVERNSY | |
| | SCSVVHEGLHNHHTTKSFSRTPGK | |
| | (SEQ ID NO: 684) | |
| I23RB8 | QVQLVQSGAEVKKPGSSVKVSCKASG | EIVLTQSPATLSLSPGERATLSCRASQS |
| | GTFKTDYINWVRQAPGQGLEWMGGII | VSSYLAWYQQKPGQAPRLLIYDASNR |
| | PIFGTANYAQKFQGRVTITADESTSTA | ATGIPARFSGSGSGTDFTLTISSLEPEDF |
| | YMELSSLRSEDTAVYYCARGPSYAYG | AVYYCQQRSNWPLTFGQGTKVEIKRA |
| | SRTYGYFDYWGQGTLVTVSSAKTTAP | DAAPTVSIFPPSSEQLTSGGASVVCFLN |
| | SVYPLAPVCGDTTGSSVTLGCLVKGYF | NFYPKDINVKWKIDGSERQNGVLNSW |
| | PEPVTLTWNSGSLSSGVHTFPAVLQSD | TDQDSKDSTYSMSSTLTLTKDEYERHN |
| | LYTLSSSVTVTSSTWPSQSITCNVAHPA | SYTCEATHKTSTSPIVKSFNRNEC (SEQ |
| | SSTKVDKKIEPRGPTIKPCPPCKCPAPN | ID NO: 687) |
| | LLGGPSVFIFPPKIKDVLMISLSPIVTCV | |
| | VVDVSEDDPDVQISWFVNNVEVHTAQ | |
| | TQTHREDYNSTLRVVSALPIQHQDWM | |
| | SGKEFKCKVNNKDLPAPIERTISKPKGS | |
| | VRAPQVYVLPPPEEEMTKKQVTLTCM | |
| | VTDFMPEDIYVEWTNNGKTELNYKNT | |
| | EPVLDSDGSYFMYSKLRVEKKNWVER | |
| | NSYSCSVVHEGLHNHHTTKSFSRTPGK | |
| | (SEQ ID NO: 686) | |
| I23RB9 | QVQLVQSGAEVKKPGSSVKVSCKASG | EIVLTQSPATLSLSPGERATLSCRASQS |
| | GTFSSYAISWVRQAPGQGLEWMGGIIP | VSSYLAWYQQKPGQAPRLLIYDASNR |
| | IFGTANYAQKFQGRVTITADESTSTAY | ATGIPARFSGSGSGTDFTLTISSLEPEDF |
| | MELSSLRSEDTAVYYCARDRSYSYGLS | AVYYCQQRSNWPLTFGQGTKVEIKRA |
| | GLGYYGFDYWGQGTLVTVSSAKTTAP | DAAPTVSIFPPSSEQLTSGGASVVCFLN |
| | SVYPLAPVCGDTTGSSVTLGCLVKGYF | NFYPKDINVKWKIDGSERQNGVLNSW |
| | PEPVTLTWNSGSLSSGVHTFPAVLQSD | TDQDSKDSTYSMSSTLTLTKDEYERHN |
| | LYTLSSSVTVTSSTWPSQSITCNVAHPA | SYTCEATHKTSTSPIVKSFNRNEC (SEQ |
| | SSTKVDKKIEPRGPTIKPCPPCKCPAPN | ID NO: 689) |
| | LLGGPSVFIFPPKIKDVLMISLSPIVTCV | |
| | VVDVSEDDPDVQISWFVNNVEVHTAQ | |
| | TQTHREDYNSTLRVVSALPIQHQDWM | |
| | SGKEFKCKVNNKDLPAPIERTISKPKGS | |
| | VRAPQVYVLPPPEEEMTKKQVTLTCM | |
| | VTDFMPEDIYVEWTNNGKTELNYKNT | |
| | EPVLDSDGSYFMYSKLRVEKKNWVER | |
| | NSYSCSVVHEGLHNHHTTKSFSRTPGK | |
| | (SEQ ID NO: 688) | |
| I23RB10 | QVQLVQSGAEVKKPGSSVKVSCKASG | DIVMTQSPDSLAVSLGERATINCKSSQS |
| | GTFSSYAIHWVRQAPGQGLEWMGRIIP | VLYSSNNKNYLAWYQQKPGQPPKLLI |
| | ISGRANYAQKFQGRVTITADESTSTAY | YWASTRESGVPDRFSGSGSGTDFTLTIS |
| | MELSSLRSEDTAVYYCARDSYPSYGSG | SLQAEDVAVYYCQQYYSTPLTFGQGT |
| | GAFGYWGQGTLVTVSSAKTTAPSVYP | KVEIKRADAAPTVSIFPPSSEQLTSGGA |
| | LAPVCGDTTGSSVTLGCLVKGYFPEPV | SVVCFLNNFYPKDINVKWKIDGSERQN |
| | TLTWNSGSLSSGVHTFPAVLQSDLYTL | GVLNSWTDQDSKDSTYSMSSTLTLTK |
| | SSSVTVTSSTWPSQSITCNVAHPASSTK | DEYERHNSYTCEATHKTSTSPIVKSFN |
| | VDKKIEPRGPTIKPCPPCKCPAPNLLGG | RNEC (SEQ ID NO: 691) |
| | PSVFIFPPKIKDVLMISLSPIVTCVVVDV | |
| | SEDDPDVQISWFVNNVEVHTAQTQTH | |
| | REDYNSTLRVVSALPIQHQDWMSGKE | |
| | FKCKVNNKDLPAPIERTISKPKGSVRAP | |
| | QVYVLPPPEEEMTKKQVTLTCMVTDF | |
| | MPEDIYVEWTNNGKTELNYKNTEPVL | |
| | DSDGSYFMYSKLRVEKKNWVERNSYS | |
| | CSVVHEGLHNHHTTKSFSRTPGK (SEQ | |
| | ID NO: 690) | |
| I23RB25 | QVQLVQSGAEVKKPGSSVKVSCKASG | DIQMTQSPSSLSASVGDRVTITCRASQS |
| 9 | GTFSSYGISWVRQAPGQGLEWMGWIS | ISNDLNWYQQKPGKAPKLLIYAASSLQ |
| | PIFGTTNYAQKFQGRVTITADESTSTAY | SGVPSRFSGSGSGTDFTLTISSLQPEDF |
| | MELSSLRSEDTAVYYCARMMMQSLD | ATYYCQQSYSTPLTFGQGTKVEIKRAD |
| | YWGQGTLVTVSSAKTTAPSVYPLAPV | AAPTVSIFPPSSEQLTSGGASVVCFLNN |
| | CGDTTGSSVTLGCLVKGYFPEPVTLTW | FYPKDINVKWKIDGSERQNGVLNSWT |
| | NSGSLSSGVHTFPAVLQSDLYTLSSSVT | DQDSKDSTYSMSSTLTLTKDEYERHNS |
| | VTSSTWPSQSITCNVAHPASSTKVDKKI | YTCEATHKTSTSPIVKSFNRNEC (SEQ |
| | EPRGPTIKPCPPCKCPAPNLLGGPSVFIF | ID NO: 693) |
| | PPKIKDVLMISLSPIVTCVVVDVSEDDP | |
| | DVQISWFVNNVEVHTAQTQTHREDYN | |
| | STLRVVSALPIQHQDWMSGKEFKCKV | |
| | NNKDLPAPIERTISKPKGSVRAPQVYV | |
| | LPPPEEEMTKKQVTLTCMVTDFMPEDI | |
| | YVEWTNNGKTELNYKNTEPVLDSDGS | |
| | YFMYSKLRVEKKNWVERNSYSCSVVH | |
| | EGLHNHHTTKSFSRTPGK (SEQ ID | |
| | NO: 692) | |
| I23RB26 | QVQLVQSGAEVKKPGSSVKVSCKASG | DIVMTQSPDSLAVSLGERATINCKSSQS |
| 0 | GTFSSYGISWVRQAPGQGLEWMGGIIP | VLYSSNNKNYLAWYQQKPGQPPKLLI |
| | IFGTANYAQKFQGRVTITADESTSTAY | YSASTRESGVPDRFSGSGSGTDFTLTIS |
| | MELSSLRSEDTAVYYCARDFGWSLWF | SLQAEDVAVYYCQQSFHVPWTFGQGT |
| | DYWGQGTLVTVSSAKTTAPSVYPLAP | KVEIKRADAAPTVSIFPPSSEQLTSGGA |
| | VCGDTTGSSVTLGCLVKGYFPEPVTLT | SVVCFLNNFYPKDINVKWKIDGSERQN |
| | WNSGSLSSGVHTFPAVLQSDLYTLSSS | GVLNSWTDQDSKDSTYSMSSTLTLTK |
| | VTVTSSTWPSQSITCNVAHPASSTKVD | DEYERHNSYTCEATHKTSTSPIVKSFN |
| | KKIEPRGPTIKPCPPCKCPAPNLLGGPS | RNEC (SEQ ID NO: 695) |
| | VFIFPPKIKDVLMISLSPIVTCVVVDVSE | |
| | DDPDVQISWFVNNVEVHTAQTQTHRE | |
| | DYNSTLRVVSALPIQHQDWMSGKEFK | |
| | CKVNNKDLPAPIERTISKPKGSVRAPQ | |
| | VYVLPPPEEEMTKKQVTLTCMVTDFM | |
| | PEDIYVEWTNNGKTELNYKNTEPVLDS | |
| | DGSYFMYSKLRVEKKNWVERNSYSCS | |
| | VVHEGLHNHHTTKSFSRTPGK (SEQ ID | |
| | NO: 694) | |
| I23RB26 | QVQLVQSGAEVKKPGSSVKVSCKASG | DIQMTQSPSSLSASVGDRVTITCRASQS |
| 1 | GTFSSYAISWVRQAPGQGLEWMGWIS | INDDLNWYQQKPGKAPKLLIYAASSL |
| | PYFGTANYAQKFQGRVTITADESTSTA | QSGVPSRFSGSGSGTDFTLTISSLQPED |
| | YMELSSLRSEDTAVYYCARMYRQGFD | FATYYCQQSYSTPLTFGQGTKVEIKRA |
| | YWGQGTLVTVSSAKTTAPSVYPLAPV | DAAPTVSIFPPSSEQLTSGGASVVCFLN |
| | CGDTTGSSVTLGCLVKGYFPEPVTLTW | NFYPKDINVKWKIDGSERQNGVLNSW |
| | NSGSLSSGVHTFPAVLQSDLYTLSSSVT | TDQDSKDSTYSMSSTLTLTKDEYERHN |
| | VTSSTWPSQSITCNVAHPASSTKVDKKI | SYTCEATHKTSTSPIVKSFNRNEC (SEQ |
| | EPRGPTIKPCPPCKCPAPNLLGGPSVFIF | ID NO: 697) |
| | PPKIKDVLMISLSPIVTCVVVDVSEDDP | |
| | DVQISWFVNNVEVHTAQTQTHREDYN | |
| | STLRVVSALPIQHQDWMSGKEFKCKV | |
| | NNKDLPAPIERTISKPKGSVRAPQVYV | |
| | LPPPEEEMTKKQVTLTCMVTDFMPEDI | |
| | YVEWTNNGKTELNYKNTEPVLDSDGS | |
| | YFMYSKLRVEKKNWVERNSYSCSVVH | |
| | EGLHNHHTTKSFSRTPGK (SEQ ID | |
| | NO: 696) | |
| I23RB26 | QVQLVQSGAEVKKPGSSVKVSCKASG | DIQMTQSPSSLSASVGDRVTITCRASQS |
| 2 | GTFSSYGISWVRQAPGQGLEWMGWIS | INKDLNWYQQKPGKAPKLLIYFASSLQ |
| | AIFGTANYAQKFQGRVTITADESTSTA | SGVPSRFSGSGSGTDFTLTISSLQPEDF |
| | YMELSSLRSEDTAVYYCARMLKQGFD | ATYYCQQSYSTPLTFGQGTKVEIKRAD |
| | YWGQGTLVTVSSAKTTAPSVYPLAPV | AAPTVSIFPPSSEQLTSGGASVVCFLNN |
| | CGDTTGSSVTLGCLVKGYFPEPVTLTW | FYPKDINVKWKIDGSERQNGVLNSWT |
| | NSGSLSSGVHTFPAVLQSDLYTLSSSVT | DQDSKDSTYSMSSTLTLTKDEYERHNS |
| | VTSSTWPSQSITCNVAHPASSTKVDKKI | YTCEATHKTSTSPIVKSFNRNEC (SEQ |
| | EPRGPTIKPCPPCKCPAPNLLGGPSVFIF | ID NO: 699) |
| | PPKIKDVLMISLSPIVTCVVVDVSEDDP | |
| | DVQISWFVNNVEVHTAQTQTHREDYN | |
| | STLRVVSALPIQHQDWMSGKEFKCKV | |
| | NNKDLPAPIERTISKPKGSVRAPQVYV | |
| | LPPPEEEMTKKQVTLTCMVTDFMPEDI | |
| | YVEWTNNGKTELNYKNTEPVLDSDGS | |
| | YFMYSKLRVEKKNWVERNSYSCSVVH | |
| | EGLHNHHTTKSFSRTPGK (SEQ ID | |
| | NO: 698) | |
| I23RB26 | QVQLVQSGAEVKKPGSSVKVSCKASG | DIQMTQSPSSLSASVGDRVTITCRASQS |
| 3 | GTFSSYAISWVRQAPGQGLEWMGGIIP | ISSYLNWYQQKPGKAPKLLIYAASSLQ |
| | IFGTANYAQKFQGRVTITADESTSTAY | SGVPSRFSGSGSGTDFTLTISSLQPEDF |
| | MELSSLRSEDTAVYYCARFHRFWLDY | ATYYCQQPYSVPFTFGQGTKVEIKRAD |
| | WGQGTLVTVSSAKTTAPSVYPLAPVC | AAPTVSIFPPSSEQLTSGGASVVCFLNN |
| | GDTTGSSVTLGCLVKGYFPEPVTLTWN | FYPKDINVKWKIDGSERQNGVLNSWT |
| | SGSLSSGVHTFPAVLQSDLYTLSSSVTV | DQDSKDSTYSMSSTLTLTKDEYERHNS |
| | TSSTWPSQSITCNVAHPASSTKVDKKIE | YTCEATHKTSTSPIVKSFNRNEC (SEQ |
| | PRGPTIKPCPPCKCPAPNLLGGPSVFIFP | ID NO: 701) |
| | PKIKDVLMISLSPIVTCVVVDVSEDDPD | |
| | VQISWFVNNVEVHTAQTQTHREDYNS | |
| | TLRVVSALPIQHQDWMSGKEFKCKVN | |
| | NKDLPAPIERTISKPKGSVRAPQVYVLP | |
| | PPEEEMTKKQVTLTCMVTDFMPEDIY | |
| | VEWTNNGKTELNYKNTEPVLDSDGSY | |
| | FMYSKLRVEKKNWVERNSYSCSVVHE | |
| | GLHNHHTTKSFSRTPGK (SEQ ID | |
| | NO: 700) | |
| I23RB26 | QVQLVQSGAEVKKPGSSVKVSCKASG | DIQMTQSPSSLSASVGDRVTITCRASQS |
| 4 | GTFSSYAISWVRQAPGQGLEWMGGIIP | IDNDLNWYQQKPGKAPKLLIYNASSL |
| | IFGTANYAQKFQGRVTITADESTSTAY | QSGVPSRFSGSGSGTDFTLTISSLQPED |
| | MELSSLRSEDTAVYYCARRSSPGFDY | FATYYCQQPYSIPLTFGQGTKVEIKRA |
| | WGQGTLVTVSSAKTTAPSVYPLAPVC | DAAPTVSIFPPSSEQLTSGGASVVCFLN |
| | GDTTGSSVTLGCLVKGYFPEPVTLTWN | NFYPKDINVKWKIDGSERQNGVLNSW |
| | SGSLSSGVHTFPAVLQSDLYTLSSSVTV | TDQDSKDSTYSMSSTLTLTKDEYERHN |
| | TSSTWPSQSITCNVAHPASSTKVDKKIE | SYTCEATHKTSTSPIVKSFNRNEC (SEQ |
| | PRGPTIKPCPPCKCPAPNLLGGPSVFIFP | ID NO: 703) |
| | PKIKDVLMISLSPIVTCVVVDVSEDDPD | |
| | VQISWFVNNVEVHTAQTQTHREDYNS | |
| | TLRVVSALPIQHQDWMSGKEFKCKVN | |
| | NKDLPAPIERTISKPKGSVRAPQVYVLP | |
| | PPEEEMTKKQVTLTCMVTDFMPEDIY | |
| | VEWTNNGKTELNYKNTEPVLDSDGSY | |
| | FMYSKLRVEKKNWVERNSYSCSVVHE | |
| | GLHNHHTTKSFSRTPGK (SEQ ID | |
| | NO: 702) | |
| I23RB26 | QVQLVQSGAEVKKPGSSVKVSCKASG | DIQMTQSPSSLSASVGDRVTITCRASQS |
| 5 | GTFSSYAISWVRQAPGQGLEWMGGIIP | ISTWLNWYQQKPGKAPKLLIYKASSLQ |
| | IFGTANYAQKFQGRVTITADESTSTAY | SGVPSRFSGSGSGTDFTLTISSLQPEDF |
| | MELSSLRSEDTAVYYCARGFVPTQGSL | ATYYCQQYNSPPLTFGQGTKVEIKRAD |
| | DYWGQGTLVTVSSAKTTAPSVYPLAP | AAPTVSIFPPSSEQLTSGGASVVCFLNN |
| | VCGDTTGSSVTLGCLVKGYFPEPVTLT | FYPKDINVKWKIDGSERQNGVLNSWT |
| | WNSGSLSSGVHTFPAVLQSDLYTLSSS | DQDSKDSTYSMSSTLTLTKDEYERHNS |
| | VTVTSSTWPSQSITCNVAHPASSTKVD | YTCEATHKTSTSPIVKSFNRNEC (SEQ |
| | KKIEPRGPTIKPCPPCKCPAPNLLGGPS | ID NO: 705) |
| | VFIFPPKIKDVLMISLSPIVTCVVVDVSE | |
| | DDPDVQISWFVNNVEVHTAQTQTHRE | |
| | DYNSTLRVVSALPIQHQDWMSGKEFK | |
| | CKVNNKDLPAPIERTISKPKGSVRAPQ | |
| | VYVLPPPEEEMTKKQVTLTCMVTDFM | |
| | PEDIYVEWTNNGKTELNYKNTEPVLDS | |
| | DGSYFMYSKLRVEKKNWVERNSYSCS | |
| | VVHEGLHNHHTTKSFSRTPGK (SEQ ID | |
| | NO: 704) | |
| I23RB26 | EVQLLESGGGLVQPGGSLRLSCAASGF | DIQMTQSPSSLSASVGDRVTITCRASQS |
| 6 | TFSDYSMSWVRQAPGKGLEWVSVISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCAKHSMAGNS | ATYYCQQSYSTPLTFGQGTKVEIKRAD |
| | FLRFLDYWGQGTLVTVSSAKTTAPSV | AAPTVSIFPPSSEQLTSGGASVVCFLNN |
| | YPLAPVCGDTTGSSVTLGCLVKGYFPE | FYPKDINVKWKIDGSERQNGVLNSWT |
| | PVTLTWNSGSLSSGVHTFPAVLQSDLY | DQDSKDSTYSMSSTLTLTKDEYERHNS |
| | TLSSSVTVTSSTWPSQSITCNVAHPASS | YTCEATHKTSTSPIVKSFNRNEC (SEQ |
| | TKVDKKIEPRGPTIKPCPPCKCPAPNLL | ID NO: 707) |
| | GGPSVFIFPPKIKDVLMISLSPIVTCVVV | |
| | DVSEDDPDVQISWFVNNVEVHTAQTQ | |
| | THREDYNSTLRVVSALPIQHQDWMSG | |
| | KEFKCKVNNKDLPAPIERTISKPKGSVR | |
| | APQVYVLPPPEEEMTKKQVTLTCMVT | |
| | DFMPEDIYVEWTNNGKTELNYKNTEP | |
| | VLDSDGSYFMYSKLRVEKKNWVERNS | |
| | YSCSVVHEGLHNHHTTKSFSRTPGK | |
| | (SEQ ID NO: 706) | |
| I23RB26 | EVQLVQSGAEVKKPGESLKISCKGSGY | EIVLTQSPGTLSLSPGERATLSCRASQS |
| 7 | SFTSYWIGWVRQMPGKGLEWMGIIYP | VSSSYLAWYQQKPGQAPRLLIYGASSR |
| | GDSDTRYSPSFQGQVTISADKSISTAYL | ATGIPDRFSGSGSGTDFTLTISRLEPEDF |
| | QWSSLKASDTAMYYCARVWYSSWDL | AVYYCQQYGSSPLTFGQGTKVEIKRA |
| | DYWGQGTLVTVSSAKTTAPSVYPLAP | DAAPTVSIFPPSSEQLTSGGASVVCFLN |
| | VCGDTTGSSVTLGCLVKGYFPEPVTLT | NFYPKDINVKWKIDGSERQNGVLNSW |
| | WNSGSLSSGVHTFPAVLQSDLYTLSSS | TDQDSKDSTYSMSSTLTLTKDEYERHN |
| | VTVTSSTWPSQSITCNVAHPASSTKVD | SYTCEATHKTSTSPIVKSFNRNEC (SEQ |
| | KKIEPRGPTIKPCPPCKCPAPNLLGGPS | ID NO: 709) |
| | VFIFPPKIKDVLMISLSPIVTCVVVDVSE | |
| | DDPDVQISWFVNNVEVHTAQTQTHRE | |
| | DYNSTLRVVSALPIQHQDWMSGKEFK | |
| | CKVNNKDLPAPIERTISKPKGSVRAPQ | |
| | VYVLPPPEEEMTKKQVTLTCMVTDFM | |
| | PEDIYVEWTNNGKTELNYKNTEPVLDS | |
| | DGSYFMYSKLRVEKKNWVERNSYSCS | |
| | VVHEGLHNHHTTKSFSRTPGK (SEQ ID | |
| | NO: 708) | |
| I23RB26 | QVQLVQSGAEVKKPGSSVKVSCKASG | EIVLTQSPATLSLSPGERATLSCRASQS |
| 8 | GTFSSYYIHWVRQAPGQGLEWMGGIIP | VSSYLAWYQQKPGQAPRLLIYDASNR |
| | IFGYANYAQKFQGRVTITADESTSTAY | ATGIPARFSGSGSGTDFTLTISSLEPEDF |
| | MELSSLRSEDTAVYYCARDPGSYGSV | AVYYCQQRSNWPLTFGQGTKVEIKRA |
| | YYRRSYYFDYWGQGTLVTVSSAKTTA | DAAPTVSIFPPSSEQLTSGGASVVCFLN |
| | PSVYPLAPVCGDTTGSSVTLGCLVKGY | NFYPKDINVKWKIDGSERQNGVLNSW |
| | FPEPVTLTWNSGSLSSGVHTFPAVLQS | TDQDSKDSTYSMSSTLTLTKDEYERHN |
| | DLYTLSSSVTVTSSTWPSQSITCNVAHP | SYTCEATHKTSTSPIVKSFNRNEC (SEQ |
| | ASSTKVDKKIEPRGPTIKPCPPCKCPAP | ID NO: 711) |
| | NLLGGPSVFIFPPKIKDVLMISLSPIVTC | |
| | VVVDVSEDDPDVQISWFVNNVEVHTA | |
| | QTQTHREDYNSTLRVVSALPIQHQDW | |
| | MSGKEFKCKVNNKDLPAPIERTISKPK | |
| | GSVRAPQVYVLPPPEEEMTKKQVTLTC | |
| | MVTDFMPEDIYVEWTNNGKTELNYKN | |
| | TEPVLDSDGSYFMYSKLRVEKKNWVE | |
| | RNSYSCSVVHEGLHNHHTTKSFSRTPG | |
| | K (SEQ ID NO: 710) | |
| I23RB26 | QVQLVQSGAEVKKPGSSVKVSCKASG | EIVLTQSPGTLSLSPGERATLSCRASQS |
| 9 | GTFSDASISWVRQAPGQGLEWMGTIIPI | VSSSYLAWYQQKPGQAPRLLIYGASSR |
| | SGNANYAQKFQGRVTITADESTSTAY | ATGIPDRFSGSGSGTDFTLTISRLEPEDF |
| | MELSSLRSEDTAVYYCARDRYVWYY | AVYYCQQYGSSPLTFGQGTKVEIKRA |
| | WDGYFDYWGQGTLVTVSSAKTTAPSV | DAAPTVSIFPPSSEQLTSGGASVVCFLN |
| | YPLAPVCGDTTGSSVTLGCLVKGYFPE | NFYPKDINVKWKIDGSERQNGVLNSW |
| | PVTLTWNSGSLSSGVHTFPAVLQSDLY | TDQDSKDSTYSMSSTLTLTKDEYERHN |
| | TLSSSVTVTSSTWPSQSITCNVAHPASS | SYTCEATHKTSTSPIVKSFNRNEC (SEQ |
| | TKVDKKIEPRGPTIKPCPPCKCPAPNLL | ID NO: 713) |
| | GGPSVFIFPPKIKDVLMISLSPIVTCVVV | |
| | DVSEDDPDVQISWFVNNVEVHTAQTQ | |
| | THREDYNSTLRVVSALPIQHQDWMSG | |
| | KEFKCKVNNKDLPAPIERTISKPKGSVR | |
| | APQVYVLPPPEEEMTKKQVTLTCMVT | |
| | DFMPEDIYVEWTNNGKTELNYKNTEP | |
| | VLDSDGSYFMYSKLRVEKKNWVERNS | |
| | YSCSVVHEGLHNHHTTKSFSRTPGK | |
| | (SEQ ID NO: 712) | |
| I23RB27 | QVQLVQSGAEVKKPGSSVKVSCKASG | EIVLTQSPATLSLSPGERATLSCRASQS |
| 0 | GTFDSYAISWVRQAPGQGLEWMGGIIP | VSSYLAWYQQKPGQAPRLLIYDASNR |
| | ISGYANYAQKFQGRVTITADESTSTAY | ATGIPARFSGSGSGTDFTLTISSLEPEDF |
| | MELSSLRSEDTAVYYCARDLSGGYLG | AVYYCQQRSNWPLTFGQGTKVEIKRA |
| | LRTLYYYYFDYWGQGTLVTVSSAKTT | DAAPTVSIFPPSSEQLTSGGASVVCFLN |
| | APSVYPLAPVCGDTTGSSVTLGCLVKG | NFYPKDINVKWKIDGSERQNGVLNSW |
| | YFPEPVTLTWNSGSLSSGVHTFPAVLQ | TDQDSKDSTYSMSSTLTLTKDEYERHN |
| | SDLYTLSSSVTVTSSTWPSQSITCNVAH | SYTCEATHKTSTSPIVKSFNRNEC (SEQ |
| | PASSTKVDKKIEPRGPTIKPCPPCKCPA | ID NO: 715) |
| | PNLLGGPSVFIFPPKIKDVLMISLSPIVT | |
| | CVVVDVSEDDPDVQISWFVNNVEVHT | |
| | AQTQTHREDYNSTLRVVSALPIQHQD | |
| | WMSGKEFKCKVNNKDLPAPIERTISKP | |
| | KGSVRAPQVYVLPPPEEEMTKKQVTL | |
| | TCMVTDFMPEDIYVEWTNNGKTELNY | |
| | KNTEPVLDSDGSYFMYSKLRVEKKNW | |
| | VERNSYSCSVVHEGLHNHHTTKSFSRT | |
| | PGK (SEQ ID NO: 714) | |
| I23RB27 | EVQLLESGGGLVQPGGSLRLSCAASGF | DIQMTQSPSSLSASVGDRVTITCRASQS |
| 1 | TFSSYAMSWVRQAPGKGLEWVSAISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCARDRGSAETF | ATYYCQQSYSTPLTFGQGTKVEIKRAD |
| | DYWGQGTLVTVSSAKTTAPSVYPLAP | AAPTVSIFPPSSEQLTSGGASVVCFLNN |
| | VCGDTTGSSVTLGCLVKGYFPEPVTLT | FYPKDINVKWKIDGSERQNGVLNSWT |
| | WNSGSLSSGVHTFPAVLQSDLYTLSSS | DQDSKDSTYSMSSTLTLTKDEYERHNS |
| | VTVTSSTWPSQSITCNVAHPASSTKVD | YTCEATHKTSTSPIVKSFNRNEC (SEQ |
| | KKIEPRGPTIKPCPPCKCPAPNLLGGPS | ID NO: 717) |
| | VFIFPPKIKDVLMISLSPIVTCVVVDVSE | |
| | DDPDVQISWFVNNVEVHTAQTQTHRE | |
| | DYNSTLRVVSALPIQHQDWMSGKEFK | |
| | CKVNNKDLPAPIERTISKPKGSVRAPQ | |
| | VYVLPPPEEEMTKKQVTLTCMVTDFM | |
| | PEDIYVEWTNNGKTELNYKNTEPVLDS | |
| | DGSYFMYSKLRVEKKNWVERNSYSCS | |
| | VVHEGLHNHHTTKSFSRTPGK (SEQ ID | |
| | NO: 716) | |
| I23RB27 | EVQLLESGGGLVQPGGSLRLSCAASGF | EIVLTQSPGTLSLSPGERATLSCRASQS |
| 2 | TFSSYAMSWVRQAPGKGLEWVSAISG | VSSSYLAWYQQKPGQAPRLLIYGASSR |
| | SGGSTYYADSVKGRFTISRDNSKNTLY | ATGIPDRFSGSGSGTDFTLTISRLEPEDF |
| | LQMNSLRAEDTAVYYCARDFGEPNGG | AVYYCQQYGSSPLTFGQGTKVEIKRA |
| | FDYWGQGTLVTVSSAKTTAPSVYPLA | DAAPTVSIFPPSSEQLTSGGASVVCFLN |
| | PVCGDTTGSSVTLGCLVKGYFPEPVTL | NFYPKDINVKWKIDGSERQNGVLNSW |
| | TWNSGSLSSGVHTFPAVLQSDLYTLSS | TDQDSKDSTYSMSSTLTLTKDEYERHN |
| | SVTVTSSTWPSQSITCNVAHPASSTKV | SYTCEATHKTSTSPIVKSFNRNEC (SEQ |
| | DKKIEPRGPTIKPCPPCKCPAPNLLGGP | ID NO: 719) |
| | SVFIFPPKIKDVLMISLSPIVTCVVVDVS | |
| | EDDPDVQISWFVNNVEVHTAQTQTHR | |
| | EDYNSTLRVVSALPIQHQDWMSGKEF | |
| | KCKVNNKDLPAPIERTISKPKGSVRAP | |
| | QVYVLPPPEEEMTKKQVTLTCMVTDF | |
| | MPEDIYVEWTNNGKTELNYKNTEPVL | |
| | DSDGSYFMYSKLRVEKKNWVERNSYS | |
| | CSVVHEGLHNHHTTKSFSRTPGK (SEQ | |
| | ID NO: 718) | |
| I23RB27 | EVQLLESGGGLVQPGGSLRLSCAASGF | DIQMTQSPSSLSASVGDRVTITCRASQS |
| 3 | TFSSYAMSWVRQAPGKGLEWVSAISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCARDPSSYYYN | ATYYCQQSYSTPLTFGQGTKVEIKRAD |
| | GYLGSYAFDYWGQGTLVTVSSAKTTA | AAPTVSIFPPSSEQLTSGGASVVCFLNN |
| | PSVYPLAPVCGDTTGSSVTLGCLVKGY | FYPKDINVKWKIDGSERQNGVLNSWT |
| | FPEPVTLTWNSGSLSSGVHTFPAVLQS | DQDSKDSTYSMSSTLTLTKDEYERHNS |
| | DLYTLSSSVTVTSSTWPSQSITCNVAHP | YTCEATHKTSTSPIVKSFNRNEC (SEQ |
| | ASSTKVDKKIEPRGPTIKPCPPCKCPAP | ID NO: 721) |
| | NLLGGPSVFIFPPKIKDVLMISLSPIVTC | |
| | VVVDVSEDDPDVQISWFVNNVEVHTA | |
| | QTQTHREDYNSTLRVVSALPIQHQDW | |
| | MSGKEFKCKVNNKDLPAPIERTISKPK | |
| | GSVRAPQVYVLPPPEEEMTKKQVTLTC | |
| | MVTDFMPEDIYVEWTNNGKTELNYKN | |
| | TEPVLDSDGSYFMYSKLRVEKKNWVE | |
| | RNSYSCSVVHEGLHNHHTTKSFSRTPG | |
| | K (SEQ ID NO: 720) | |
| I23RB27 | EVQLLESGGGLVQPGGSLRLSCAASGF | DIQMTQSPSSLSASVGDRVTITCRASQS |
| 4 | TFSSYAMSWVRQAPGKGLEWVSAISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCAREDATDSSD | ATYYCQQSYSTPLTFGQGTKVEIKRAD |
| | YYFDYWGQGTLVTVSSAKTTAPSVYP | AAPTVSIFPPSSEQLTSGGASVVCFLNN |
| | LAPVCGDTTGSSVTLGCLVKGYFPEPV | FYPKDINVKWKIDGSERQNGVLNSWT |
| | TLTWNSGSLSSGVHTFPAVLQSDLYTL | DQDSKDSTYSMSSTLTLTKDEYERHNS |
| | SSSVTVTSSTWPSQSITCNVAHPASSTK | YTCEATHKTSTSPIVKSFNRNEC (SEQ |
| | VDKKIEPRGPTIKPCPPCKCPAPNLLGG | ID NO: 723) |
| | PSVFIFPPKIKDVLMISLSPIVTCVVVDV | |
| | SEDDPDVQISWFVNNVEVHTAQTQTH | |
| | REDYNSTLRVVSALPIQHQDWMSGKE | |
| | FKCKVNNKDLPAPIERTISKPKGSVRAP | |
| | QVYVLPPPEEEMTKKQVTLTCMVTDF | |
| | MPEDIYVEWTNNGKTELNYKNTEPVL | |
| | DSDGSYFMYSKLRVEKKNWVERNSYS | |
| | CSVVHEGLHNHHTTKSFSRTPGK (SEQ | |
| | ID NO: 722) | |
| I23RB27 | EVQLLESGGGLVQPGGSLRLSCAASGF | DIQMTQSPSSLSASVGDRVTITCRASQS |
| 5 | TFSDSGMSWVRQAPGKGLEWVSAIDG | ISSYLNWYQQKPGKAPKLLIYAASSLQ |
| | SGGTTYYADSVKGRFTISRDNSKNTLY | SGVPSRFSGSGSGTDFTLTISSLQPEDF |
| | LQMNSLRAEDTAVYYCARGSGGYVG | ATYYCQQSYSTPLTFGQGTKVEIKRAD |
| | YGGFDYWGQGTLVTVSSAKTTAPSVY | AAPTVSIFPPSSEQLTSGGASVVCFLNN |
| | PLAPVCGDTTGSSVTLGCLVKGYFPEP | FYPKDINVKWKIDGSERQNGVLNSWT |
| | VTLTWNSGSLSSGVHTFPAVLQSDLYT | DQDSKDSTYSMSSTLTLTKDEYERHNS |
| | LSSSVTVTSSTWPSQSITCNVAHPASST | |
| | KVDKKIEPRGPTIKPCPPCKCPAPNLLG | YTCEATHKTSTSPIVKSFNRNEC (SEQ |
| | GPSVFIFPPKIKDVLMISLSPIVTCVVVD | ID NO: 725) |
| | VSEDDPDVQISWFVNNVEVHTAQTQT | |
| | HREDYNSTLRVVSALPIQHQDWMSGK | |
| | EFKCKVNNKDLPAPIERTISKPKGSVRA | |
| | PQVYVLPPPEEEMTKKQVTLTCMVTD | |
| | FMPEDIYVEWTNNGKTELNYKNTEPV | |
| | LDSDGSYFMYSKLRVEKKNWVERNSY | |
| | SCSVVHEGLHNHHTTKSFSRTPGK | |
| | (SEQ ID NO: 724) | |
| I23RB27 | EVQLLESGGGLVQPGGSLRLSCAASGF | DIQMTQSPSSLSASVGDRVTITCRASQS |
| 6 | TFSNYAMSWVRQAPGKGLEWVSAISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCARDGSYGYR | ATYYCQQSYSTPLTFGQGTKVEIKRAD |
| | GRRRRYYDYFDYWGQGTLVTVSSAKT | AAPTVSIFPPSSEQLTSGGASVVCFLNN |
| | TAPSVYPLAPVCGDTTGSSVTLGCLVK | FYPKDINVKWKIDGSERQNGVLNSWT |
| | GYFPEPVTLTWNSGSLSSGVHTFPAVL | DQDSKDSTYSMSSTLTLTKDEYERHNS |
| | QSDLYTLSSSVTVTSSTWPSQSITCNVA | YTCEATHKTSTSPIVKSFNRNEC (SEQ |
| | HPASSTKVDKKIEPRGPTIKPCPPCKCP | ID NO: 727) |
| | APNLLGGPSVFIFPPKIKDVLMISLSPIV | |
| | TCVVVDVSEDDPDVQISWFVNNVEVH | |
| | TAQTQTHREDYNSTLRVVSALPIQHQD | |
| | WMSGKEFKCKVNNKDLPAPIERTISKP | |
| | KGSVRAPQVYVLPPPEEEMTKKQVTL | |
| | TCMVTDFMPEDIYVEWTNNGKTELNY | |
| | KNTEPVLDSDGSYFMYSKLRVEKKNW | |
| | VERNSYSCSVVHEGLHNHHTTKSFSRT | |
| | PGK (SEQ ID NO: 726) | |
| I23RB27 | EVQLLESGGGLVQPGGSLRLSCAASGF | DIQMTQSPSSLSASVGDRVTITCRASQS |
| 7 | TFSSYAMSWVRQAPGKGLEWVSAISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCARRRRYRYFD | ATYYCQQSYSTPLTFGQGTKVEIKRAD |
| | YWGQGTLVTVSSAKTTAPSVYPLAPV | AAPTVSIFPPSSEQLTSGGASVVCFLNN |
| | CGDTTGSSVTLGCLVKGYFPEPVTLTW | FYPKDINVKWKIDGSERQNGVLNSWT |
| | NSGSLSSGVHTFPAVLQSDLYTLSSSVT | DQDSKDSTYSMSSTLTLTKDEYERHNS |
| | VTSSTWPSQSITCNVAHPASSTKVDKKI | YTCEATHKTSTSPIVKSFNRNEC (SEQ |
| | EPRGPTIKPCPPCKCPAPNLLGGPSVFIF | ID NO: 729) |
| | PPKIKDVLMISLSPIVTCVVVDVSEDDP | |
| | DVQISWFVNNVEVHTAQTQTHREDYN | |
| | STLRVVSALPIQHQDWMSGKEFKCKV | |
| | NNKDLPAPIERTISKPKGSVRAPQVYV | |
| | LPPPEEEMTKKQVTLTCMVTDFMPEDI | |
| | YVEWTNNGKTELNYKNTEPVLDSDGS | |
| | YFMYSKLRVEKKNWVERNSYSCSVVH | |
| | EGLHNHHTTKSFSRTPGK (SEQ ID | |
| | NO: 728) | |
| I23RB27 | EVQLLESGGGLVQPGGSLRLSCAASGF | EIVLTQSPGTLSLSPGERATLSCRASQS |
| 8 | TFSSYAMSWVRQAPGKGLEWVSAISG | VSSSYLAWYQQKPGQAPRLLIYGASSR |
| | SGGSTYYADSVKGRFTISRDNSKNTLY | ATGIPDRFSGSGSGTDFTLTISRLEPEDF |
| | LQMNSLRAEDTAVYYCVRGGSLGGGF | AVYYCQQYGSSPLTFGQGTKVEIKRA |
| | YFDYWGQGTLVTVSSAKTTAPSVYPL | DAAPTVSIFPPSSEQLTSGGASVVCFLN |
| | APVCGDTTGSSVTLGCLVKGYFPEPVT | NFYPKDINVKWKIDGSERQNGVLNSW |
| | LTWNSGSLSSGVHTFPAVLQSDLYTLS | TDQDSKDSTYSMSSTLTLTKDEYERHN |
| | SSVTVTSSTWPSQSITCNVAHPASSTKV | SYTCEATHKTSTSPIVKSFNRNEC (SEQ |
| | DKKIEPRGPTIKPCPPCKCPAPNLLGGP | ID NO: 731) |
| | SVFIFPPKIKDVLMISLSPIVTCVVVDVS | |
| | EDDPDVQISWFVNNVEVHTAQTQTHR | |
| | EDYNSTLRVVSALPIQHQDWMSGKEF | |
| | KCKVNNKDLPAPIERTISKPKGSVRAP | |
| | QVYVLPPPEEEMTKKQVTLTCMVTDF | |
| | MPEDIYVEWTNNGKTELNYKNTEPVL | |
| | DSDGSYFMYSKLRVEKKNWVERNSYS | |
| | CSVVHEGLHNHHTTKSFSRTPGK (SEQ | |
| | ID NO: 730) | |
| I23RB27 | EVQLLESGGGLVQPGGSLRLSCAASGF | DIQMTQSPSSLSASVGDRVTITCRASQS |
| 9 | TFDTYAMHWVRQAPGKGLEWVSWIS | ISSYLNWYQQKPGKAPKLLIYAASSLQ |
| | GEGGSTEYADSVKGRFTISRDNSKNTL | SGVPSRFSGSGSGTDFTLTISSLQPEDF |
| | YLQMNSLRAEDTAVYYCARDLGAYN | ATYYCQQSYSTPLTFGQGTKVEIKRAD |
| | RPFYAFDYWGQGTLVTVSSAKTTAPS | AAPTVSIFPPSSEQLTSGGASVVCFLNN |
| | VYPLAPVCGDTTGSSVTLGCLVKGYFP | FYPKDINVKWKIDGSERQNGVLNSWT |
| | EPVTLTWNSGSLSSGVHTFPAVLQSDL | DQDSKDSTYSMSSTLTLTKDEYERHNS |
| | YTLSSSVTVTSSTWPSQSITCNVAHPAS | YTCEATHKTSTSPIVKSFNRNEC (SEQ |
| | STKVDKKIEPRGPTIKPCPPCKCPAPNL | ID NO: 733) |
| | LGGPSVFIFPPKIKDVLMISLSPIVTCVV | |
| | VDVSEDDPDVQISWFVNNVEVHTAQT | |
| | QTHREDYNSTLRVVSALPIQHQDWMS | |
| | GKEFKCKVNNKDLPAPIERTISKPKGS | |
| | VRAPQVYVLPPPEEEMTKKQVTLTCM | |
| | VTDFMPEDIYVEWTNNGKTELNYKNT | |
| | EPVLDSDGSYFMYSKLRVEKKNWVER | |
| | NSYSCSVVHEGLHNHHTTKSFSRTPGK | |
| | (SEQ ID NO: 732) | |
| I23RB28 | EVQLLESGGGLVQPGGSLRLSCAASGF | EIVLTQSPGTLSLSPGERATLSCRASQS |
| 0 | TFSNAAMHWVRQAPGKGLEWVSRID | VSSSYLAWYQQKPGQAPRLLIYGASSR |
| | GSGGYTEYADSVKGRFTISRDNSKNTL | ATGIPDRFSGSGSGTDFTLTISRLEPEDF |
| | YLQMNSLRAEDTAVYYCARELYSYLG | AVYYCQQYGSSPLTFGQGTKVEIKRA |
| | SSYYYGYFDYWGQGTLVTVSSAKTTA | DAAPTVSIFPPSSEQLTSGGASVVCFLN |
| | PSVYPLAPVCGDTTGSSVTLGCLVKGY | NFYPKDINVKWKIDGSERQNGVLNSW |
| | FPEPVTLTWNSGSLSSGVHTFPAVLQS | TDQDSKDSTYSMSSTLTLTKDEYERHN |
| | DLYTLSSSVTVTSSTWPSQSITCNVAHP | SYTCEATHKTSTSPIVKSFNRNEC (SEQ |
| | ASSTKVDKKIEPRGPTIKPCPPCKCPAP | ID NO: 735) |
| | NLLGGPSVFIFPPKIKDVLMISLSPIVTC | |
| | VVVDVSEDDPDVQISWFVNNVEVHTA | |
| | QTQTHREDYNSTLRVVSALPIQHQDW | |
| | MSGKEFKCKVNNKDLPAPIERTISKPK | |
| | GSVRAPQVYVLPPPEEEMTKKQVTLTC | |
| | MVTDFMPEDIYVEWTNNGKTELNYKN | |
| | TEPVLDSDGSYFMYSKLRVEKKNWVE | |
| | RNSYSCSVVHEGLHNHHTTKSFSRTPG | |
| | K (SEQ ID NO: 734) | |
| I23RB28 | EVQLLESGGGLVQPGGSLRLSCAASGF | DIQMTQSPSSLSASVGDRVTITCRASQS |
| 1 | TFSSYAMSWVRQAPGKGLEWVSAISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCARGPYYSGTS | ATYYCQQSYSTPLTFGQGTKVEIKRAD |
| | GYRSGGGFDYWGQGTLVTVSSAKTTA | AAPTVSIFPPSSEQLTSGGASVVCFLNN |
| | PSVYPLAPVCGDTTGSSVTLGCLVKGY | FYPKDINVKWKIDGSERQNGVLNSWT |
| | FPEPVTLTWNSGSLSSGVHTFPAVLQS | DQDSKDSTYSMSSTLTLTKDEYERHNS |
| | DLYTLSSSVTVTSSTWPSQSITCNVAHP | YTCEATHKTSTSPIVKSFNRNEC (SEQ |
| | ASSTKVDKKIEPRGPTIKPCPPCKCPAP | ID NO: 737) |
| | NLLGGPSVFIFPPKIKDVLMISLSPIVTC | |
| | VVVDVSEDDPDVQISWFVNNVEVHTA | |
| | QTQTHREDYNSTLRVVSALPIQHQDW | |
| | MSGKEFKCKVNNKDLPAPIERTISKPK | |
| | GSVRAPQVYVLPPPEEEMTKKQVTLTC | |
| | MVTDFMPEDIYVEWTNNGKTELNYKN | |
| | TEPVLDSDGSYFMYSKLRVEKKNWVE | |
| | RNSYSCSVVHEGLHNHHTTKSFSRTPG | |
| | K (SEQ ID NO: 736) | |
| I23RB28 | EVQLLESGGGLVQPGGSLRLSCAASGF | DIQMTQSPSSLSASVGDRVTITCRASQS |
| 2 | TFSNDAMNWVRQAPGKGLEWVSAID | ISSYLNWYQQKPGKAPKLLIYAASSLQ |
| | GNGGSTNYADSVKGRFTISRDNSKNTL | SGVPSRFSGSGSGTDFTLTISSLQPEDF |
| | YLQMNSLRAEDTAVYYCARDLGGYY | ATYYCQQSYSTPLTFGQGTKVEIKRAD |
| | GRRRRYYGMDVWGQGTLVTVSSAKT | AAPTVSIFPPSSEQLTSGGASVVCFLNN |
| | TAPSVYPLAPVCGDTTGSSVTLGCLVK | FYPKDINVKWKIDGSERQNGVLNSWT |
| | GYFPEPVTLTWNSGSLSSGVHTFPAVL | DQDSKDSTYSMSSTLTLTKDEYERHNS |
| | QSDLYTLSSSVTVTSSTWPSQSITCNVA | YTCEATHKTSTSPIVKSFNRNEC (SEQ |
| | HPASSTKVDKKIEPRGPTIKPCPPCKCP | ID NO: 739) |
| | APNLLGGPSVFIFPPKIKDVLMISLSPIV | |
| | TCVVVDVSEDDPDVQISWFVNNVEVH | |
| | TAQTQTHREDYNSTLRVVSALPIQHQD | |
| | WMSGKEFKCKVNNKDLPAPIERTISKP | |
| | KGSVRAPQVYVLPPPEEEMTKKQVTL | |
| | TCMVTDFMPEDIYVEWTNNGKTELNY | |
| | KNTEPVLDSDGSYFMYSKLRVEKKNW | |
| | VERNSYSCSVVHEGLHNHHTTKSFSRT | |
| | PGK (SEQ ID NO: 738) | |
| I23RB28 | EVQLLESGGGLVQPGGSLRLSCAAIGF | EIVLTQSPGTLSLSPGERATLSCRASQS |
| 3 | TFSSSAMHWVRQAPGKGLEWVSAIDG | VSSSYLAWYQQKPGQAPRLLIYGASSR |
| | EGGSTYYADSVKGRFTISRDNSKNTLY | ATGIPDRFSGSGSGTDFTLTISRLEPEDF |
| | LQMNSLRAEDTAVYYCAREVSSYGRY | AVYYCQQYGSSPLTFGQGTKVEIKRA |
| | GFGYWGQGTLVTVSSAKTTAPSVYPL | DAAPTVSIFPPSSEQLTSGGASVVCFLN |
| | APVCGDTTGSSVTLGCLVKGYFPEPVT | NFYPKDINVKWKIDGSERQNGVLNSW |
| | LTWNSGSLSSGVHTFPAVLQSDLYTLS | TDQDSKDSTYSMSSTLTLTKDEYERHN |
| | SSVTVTSSTWPSQSITCNVAHPASSTKV | SYTCEATHKTSTSPIVKSFNRNEC (SEQ |
| | DKKIEPRGPTIKPCPPCKCPAPNLLGGP | ID NO: 741) |
| | SVFIFPPKIKDVLMISLSPIVTCVVVDVS | |
| | EDDPDVQISWFVNNVEVHTAQTQTHR | |
| | EDYNSTLRVVSALPIQHQDWMSGKEF | |
| | KCKVNNKDLPAPIERTISKPKGSVRAP | |
| | QVYVLPPPEEEMTKKQVTLTCMVTDF | |
| | MPEDIYVEWTNNGKTELNYKNTEPVL | |
| | DSDGSYFMYSKLRVEKKNWVERNSYS | |
| | CSVVHEGLHNHHTTKSFSRTPGK (SEQ | |
| | ID NO: 740) | |
| I23RB28 | EVQLLESGGGLVQPGGSLRLSCAASGF | DIQMTQSPSSLSASVGDRVTITCRASQS |
| 4 | TFSSYAMSWVRQAPGKGLEWVSAISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCARGLAGNSSG | ATYYCQQSYSTPLTFGQGTKVEIKRAD |
| | GGFDYWGQGTLVTVSSAKTTAPSVYP | AAPTVSIFPPSSEQLTSGGASVVCFLNN |
| | LAPVCGDTTGSSVTLGCLVKGYFPEPV | FYPKDINVKWKIDGSERQNGVLNSWT |
| | TLTWNSGSLSSGVHTFPAVLQSDLYTL | DQDSKDSTYSMSSTLTLTKDEYERHNS |
| | SSSVTVTSSTWPSQSITCNVAHPASSTK | YTCEATHKTSTSPIVKSFNRNEC (SEQ |
| | VDKKIEPRGPTIKPCPPCKCPAPNLLGG | ID NO: 743) |
| | PSVFIFPPKIKDVLMISLSPIVTCVVVDV | |
| | SEDDPDVQISWFVNNVEVHTAQTQTH | |
| | REDYNSTLRVVSALPIQHQDWMSGKE | |
| | FKCKVNNKDLPAPIERTISKPKGSVRAP | |
| | QVYVLPPPEEEMTKKQVTLTCMVTDF | |
| | MPEDIYVEWTNNGKTELNYKNTEPVL | |
| | DSDGSYFMYSKLRVEKKNWVERNSYS | |
| | CSVVHEGLHNHHTTKSFSRTPGK (SEQ | |
| | ID NO: 742) | |
| I23RB28 | EVQLLESGGGLVQPGGSLRLSCAASGF | DIQMTQSPSSLSASVGDRVTITCRASQS |
| 5 | TFDDYAMNWVRQAPGKGLEWVSAIA | ISSYLNWYQQKPGKAPKLLIYAASSLQ |
| | GNGGSTEYADSVKGRFTISRDNSKNTL | SGVPSRFSGSGSGTDFTLTISSLQPEDF |
| | YLQMNSLRAEDTAVYYCARDGGYPLT | ATYYCQQSYSTPLTFGQGTKVEIKRAD |
| | AVTGIYFDYWGQGTLVTVSSAKTTAPS | AAPTVSIFPPSSEQLTSGGASVVCFLNN |
| | VYPLAPVCGDTTGSSVTLGCLVKGYFP | FYPKDINVKWKIDGSERQNGVLNSWT |
| | EPVTLTWNSGSLSSGVHTFPAVLQSDL | DQDSKDSTYSMSSTLTLTKDEYERHNS |
| | YTLSSSVTVTSSTWPSQSITCNVAHPAS | YTCEATHKTSTSPIVKSFNRNEC (SEQ |
| | STKVDKKIEPRGPTIKPCPPCKCPAPNL | ID NO: 745) |
| | LGGPSVFIFPPKIKDVLMISLSPIVTCVV | |
| | VDVSEDDPDVQISWFVNNVEVHTAQT | |
| | QTHREDYNSTLRVVSALPIQHQDWMS | |
| | GKEFKCKVNNKDLPAPIERTISKPKGS | |
| | VRAPQVYVLPPPEEEMTKKQVTLTCM | |
| | VTDFMPEDIYVEWTNNGKTELNYKNT | |
| | EPVLDSDGSYFMYSKLRVEKKNWVER | |
| | NSYSCSVVHEGLHNHHTTKSFSRTPGK | |
| | (SEQ ID NO: 744) | |
| I23RB28 | EVQLLESGGGLVQPGGSLRLSCAASGF | DIQMTQSPSSLSASVGDRVTITCRASQS |
| 6 | TFSDAGMSWVRQAPGKGLEWVSAISG | ISSYLNWYQQKPGKAPKLLIYAASSLQ |
| | NGGSTNYADSVKGRFTISRDNSKNTLY | SGVPSRFSGSGSGTDFTLTISSLQPEDF |
| | LQMNSLRAEDTAVYYCARRRRRASFD | ATYYCQQSYSTPLTFGQGTKVEIKRAD |
| | YWGQGTLVTVSSAKTTAPSVYPLAPV | AAPTVSIFPPSSEQLTSGGASVVCFLNN |
| | CGDTTGSSVTLGCLVKGYFPEPVTLTW | FYPKDINVKWKIDGSERQNGVLNSWT |
| | NSGSLSSGVHTFPAVLQSDLYTLSSSVT | DQDSKDSTYSMSSTLTLTKDEYERHNS |
| | VTSSTWPSQSITCNVAHPASSTKVDKKI | YTCEATHKTSTSPIVKSFNRNEC (SEQ |
| | EPRGPTIKPCPPCKCPAPNLLGGPSVFIF | ID NO: 747) |
| | PPKIKDVLMISLSPIVTCVVVDVSEDDP | |
| | DVQISWFVNNVEVHTAQTQTHREDYN | |
| | STLRVVSALPIQHQDWMSGKEFKCKV | |
| | NNKDLPAPIERTISKPKGSVRAPQVYV | |
| | LPPPEEEMTKKQVTLTCMVTDFMPEDI | |
| | YVEWTNNGKTELNYKNTEPVLDSDGS | |
| | YFMYSKLRVEKKNWVERNSYSCSVVH | |
| | EGLHNHHTTKSFSRTPGK (SEQ ID | |
| | NO: 746) | |
| I23RB28 | EVQLVQSGAEVKKPGESLKISCKGSGY | EIVLTQSPGTLSLSPGERATLSCRASQS |
| 7 | SFTDAAIHWVRQMPGKGLEWMGTIDP | VSSSYLAWYQQKPGQAPRLLIYGASSR |
| | GDSTTRYSPSFQGQVTISADKSISTAYL | ATGIPDRFSGSGSGTDFTLTISRLEPEDF |
| | QWSSLKASDTAVYYCARDLYYTLEYN | AVYYCQQYGSSPLTFGQGTKVEIKRA |
| | WYYDAFDYWGQGTLVTVSSAKTTAPS | DAAPTVSIFPPSSEQLTSGGASVVCFLN |
| | VYPLAPVCGDTTGSSVTLGCLVKGYFP | NFYPKDINVKWKIDGSERQNGVLNSW |
| | EPVTLTWNSGSLSSGVHTFPAVLQSDL | TDQDSKDSTYSMSSTLTLTKDEYERHN |
| | YTLSSSVTVTSSTWPSQSITCNVAHPAS | SYTCEATHKTSTSPIVKSFNRNEC (SEQ |
| | STKVDKKIEPRGPTIKPCPPCKCPAPNL | ID NO: 749) |
| | LGGPSVFIFPPKIKDVLMISLSPIVTCVV | |
| | VDVSEDDPDVQISWFVNNVEVHTAQT | |
| | QTHREDYNSTLRVVSALPIQHQDWMS | |
| | GKEFKCKVNNKDLPAPIERTISKPKGS | |
| | VRAPQVYVLPPPEEEMTKKQVTLTCM | |
| | VTDFMPEDIYVEWTNNGKTELNYKNT | |
| | EPVLDSDGSYFMYSKLRVEKKNWVER | |
| | NSYSCSVVHEGLHNHHTTKSFSRTPGK | |
| | (SEQ ID NO: 748) | |
| I23RB28 | EVQLVQSGAEVKKPGESLKISCKGSGY | EIVLTQSPGTLSLSPGERATLSCRASQS |
| 8 | SFKSDWISWVRQMPGKGLEWMGWIY | VSSSYLAWYQQKPGQAPRLLIYGASSR |
| | PGDSRTYYSPSFQGQVTISADKSISTAY | ATGIPDRFSGSGSGTDFTLTISRLEPEDF |
| | LQWSSLKASDTAVYYCARDSSYGTGN | AVYYCQQYGSSPLTFGQGTKVEIKRA |
| | RGGRSGAFDYWGQGTLVTVSSAKTTA | DAAPTVSIFPPSSEQLTSGGASVVCFLN |
| | PSVYPLAPVCGDTTGSSVTLGCLVKGY | NFYPKDINVKWKIDGSERQNGVLNSW |
| | FPEPVTLTWNSGSLSSGVHTFPAVLQS | TDQDSKDSTYSMSSTLTLTKDEYERHN |
| | DLYTLSSSVTVTSSTWPSQSITCNVAHP | SYTCEATHKTSTSPIVKSFNRNEC (SEQ |
| | ASSTKVDKKIEPRGPTIKPCPPCKCPAP | ID NO: 751) |
| | NLLGGPSVFIFPPKIKDVLMISLSPIVTC | |
| | VVVDVSEDDPDVQISWFVNNVEVHTA | |
| | QTQTHREDYNSTLRVVSALPIQHQDW | |
| | MSGKEFKCKVNNKDLPAPIERTISKPK | |
| | GSVRAPQVYVLPPPEEEMTKKQVTLTC | |
| | MVTDFMPEDIYVEWTNNGKTELNYKN | |
| | TEPVLDSDGSYFMYSKLRVEKKNWVE | |
| | RNSYSCSVVHEGLHNHHTTKSFSRTPG | |
| | K (SEQ ID NO: 750) | |
| I23RB28 | EVQLVQSGAEVKKPGESLKISCKGSGY | DIQMTQSPSSLSASVGDRVTITCRASQS |
| 9 | SFDSAGINWVRQMPGKGLEWMGNIYP | ISSYLNWYQQKPGKAPKLLIYAASSLQ |
| | GDSDTRYSPSFQGQVTISADKSISTAYL | SGVPSRFSGSGSGTDFTLTISSLQPEDF |
| | QWSSLKASDTAVYYCARGGGSLGFDY | ATYYCQQSYSTPLTFGQGTKVEIKRAD |
| | WGQGTLVTVSSAKTTAPSVYPLAPVC | AAPTVSIFPPSSEQLTSGGASVVCFLNN |
| | GDTTGSSVTLGCLVKGYFPEPVTLTWN | FYPKDINVKWKIDGSERQNGVLNSWT |
| | SGSLSSGVHTFPAVLQSDLYTLSSSVTV | DQDSKDSTYSMSSTLTLTKDEYERHNS |
| | TSSTWPSQSITCNVAHPASSTKVDKKIE | YTCEATHKTSTSPIVKSFNRNEC (SEQ |
| | PRGPTIKPCPPCKCPAPNLLGGPSVFIFP | ID NO: 753) |
| | PKIKDVLMISLSPIVTCVVVDVSEDDPD | |
| | VQISWFVNNVEVHTAQTQTHREDYNS | |
| | TLRVVSALPIQHQDWMSGKEFKCKVN | |
| | NKDLPAPIERTISKPKGSVRAPQVYVLP | |
| | PPEEEMTKKQVTLTCMVTDFMPEDIY | |
| | VEWTNNGKTELNYKNTEPVLDSDGSY | |
| | FMYSKLRVEKKNWVERNSYSCSVVHE | |
| | GLHNHHTTKSFSRTPGK (SEQ ID | |
| | NO: 752) | |
| I23RB29 | EVQLVQSGAEVKKPGESLKISCKGSGY | DIQMTQSPSSLSASVGDRVTITCRASQS |
| 0 | SFKNDGIHWVRQMPGKGLEWMGWIA | ISSYLNWYQQKPGKAPKLLIYAASSLQ |
| | PGESTTGYSPSFQGQVTISADKSISTAY | SGVPSRFSGSGSGTDFTLTISSLQPEDF |
| | LQWSSLKASDTAVYYCARDLGGYGSA | ATYYCQQSYSTPLTFGQGTKVEIKRAD |
| | SGRRSDYFDYWGQGTLVTVSSAKTTA | AAPTVSIFPPSSEQLTSGGASVVCFLNN |
| | PSVYPLAPVCGDTTGSSVTLGCLVKGY | FYPKDINVKWKIDGSERQNGVLNSWT |
| | FPEPVTLTWNSGSLSSGVHTFPAVLQS | DQDSKDSTYSMSSTLTLTKDEYERHNS |
| | DLYTLSSSVTVTSSTWPSQSITCNVAHP | YTCEATHKTSTSPIVKSFNRNEC (SEQ |
| | ASSTKVDKKIEPRGPTIKPCPPCKCPAP | ID NO: 755) |
| | NLLGGPSVFIFPPKIKDVLMISLSPIVTC | |
| | VVVDVSEDDPDVQISWFVNNVEVHTA | |
| | QTQTHREDYNSTLRVVSALPIQHQDW | |
| | MSGKEFKCKVNNKDLPAPIERTISKPK | |
| | GSVRAPQVYVLPPPEEEMTKKQVTLTC | |
| | MVTDFMPEDIYVEWTNNGKTELNYKN | |
| | TEPVLDSDGSYFMYSKLRVEKKNWVE | |
| | RNSYSCSVVHEGLHNHHTTKSFSRTPG | |
| | K (SEQ ID NO: 754) | |
| I23RB29 | EVQLVQSGAEVKKPGESLKISCKGSGY | EIVLTQSPATLSLSPGERATLSCRASQS |
| 1 | SFTSYYISWVRQMPGKGLEWMGNIYP | VSSYLAWYQQKPGQAPRLLIYDASNR |
| | GDSRTRYSPSFQGQVTISADKSISTAYL | ATGIPARFSGSGSGTDFTLTISSLEPEDF |
| | QWSSLKASDTAVYYCARDRYYGIPFD | AVYYCQQRSNWPLTFGQGTKVEIKRA |
| | YWGQGTLVTVSSAKTTAPSVYPLAPV | DAAPTVSIFPPSSEQLTSGGASVVCFLN |
| | CGDTTGSSVTLGCLVKGYFPEPVTLTW | NFYPKDINVKWKIDGSERQNGVLNSW |
| | NSGSLSSGVHTFPAVLQSDLYTLSSSVT | TDQDSKDSTYSMSSTLTLTKDEYERHN |
| | VTSSTWPSQSITCNVAHPASSTKVDKKI | SYTCEATHKTSTSPIVKSFNRNEC (SEQ |
| | EPRGPTIKPCPPCKCPAPNLLGGPSVFIF | ID NO: 757) |
| | PPKIKDVLMISLSPIVTCVVVDVSEDDP | |
| | DVQISWFVNNVEVHTAQTQTHREDYN | |
| | STLRVVSALPIQHQDWMSGKEFKCKV | |
| | NNKDLPAPIERTISKPKGSVRAPQVYV | |
| | LPPPEEEMTKKQVTLTCMVTDFMPEDI | |
| | YVEWTNNGKTELNYKNTEPVLDSDGS | |
| | YFMYSKLRVEKKNWVERNSYSCSVVH | |
| | EGLHNHHTTKSFSRTPGK (SEQ ID | |
| | NO: 756) | |
| I23RB29 | EVQLVQSGAEVKKPGESLKISCKGSGY | DIQMTQSPSSLSASVGDRVTITCRASQS |
| 2 | SFDTDYINWVRQMPGKGLEWMGYILP | ISSYLNWYQQKPGKAPKLLIYAASSLQ |
| | GESDTRYSPSFQGQVTISADKSISTAYL | SGVPSRFSGSGSGTDFTLTISSLQPEDF |
| | QWSSLKASDTAVYYCARGGGYGLVN | ATYYCQQSYSTPLTFGQGTKVEIKRAD |
| | RRRRSYAFDYWGQGTLVTVSSAKTTA | AAPTVSIFPPSSEQLTSGGASVVCFLNN |
| | PSVYPLAPVCGDTTGSSVTLGCLVKGY | FYPKDINVKWKIDGSERQNGVLNSWT |
| | FPEPVTLTWNSGSLSSGVHTFPAVLQS | DQDSKDSTYSMSSTLTLTKDEYERHNS |
| | DLYTLSSSVTVTSSTWPSQSITCNVAHP | YTCEATHKTSTSPIVKSFNRNEC (SEQ |
| | ASSTKVDKKIEPRGPTIKPCPPCKCPAP | ID NO: 759) |
| | NLLGGPSVFIFPPKIKDVLMISLSPIVTC | |
| | VVVDVSEDDPDVQISWFVNNVEVHTA | |
| | QTQTHREDYNSTLRVVSALPIQHQDW | |
| | MSGKEFKCKVNNKDLPAPIERTISKPK | |
| | GSVRAPQVYVLPPPEEEMTKKQVTLTC | |
| | MVTDFMPEDIYVEWTNNGKTELNYKN | |
| | TEPVLDSDGSYFMYSKLRVEKKNWVE | |
| | RNSYSCSVVHEGLHNHHTTKSFSRTPG | |
| | K (SEQ ID NO: 758) | |
| I23RB31 | QVQLVQSGAEVKKPGSSVKVSCKASG | DIQMTQSPSSLSASVGDRVTITCRASQS |
| 3 | GTFSSYGISWVRQAPGQGLEWMGWIS | IGEDLNWYQQKPGKAPKLLIYAASSLQ |
| | AIFGTTNYAQKFQGRVTITADESTSTA | SGVPSRFSGSGSGTDFTLTISSLQPEDF |
| | YMELSSLRSEDTAVYYCARGTVPGFD | ATYYCQQSYSTPLTFGQGTKVEIKRAD |
| | YWGQGTLVTVSSAKTTAPSVYPLAPV | AAPTVSIFPPSSEQLTSGGASVVCFLNN |
| | CGDTTGSSVTLGCLVKGYFPEPVTLTW | FYPKDINVKWKIDGSERQNGVLNSWT |
| | NSGSLSSGVHTFPAVLQSDLYTLSSSVT | DQDSKDSTYSMSSTLTLTKDEYERHNS |
| | VTSSTWPSQSITCNVAHPASSTKVDKKI | YTCEATHKTSTSPIVKSFNRNEC (SEQ |
| | EPRGPTIKPCPPCKCPAPNLLGGPSVFIF | ID NO: 761) |
| | PPKIKDVLMISLSPIVTCVVVDVSEDDP | |
| | DVQISWFVNNVEVHTAQTQTHREDYN | |
| | STLRVVSALPIQHQDWMSGKEFKCKV | |
| | NNKDLPAPIERTISKPKGSVRAPQVYV | |
| | LPPPEEEMTKKQVTLTCMVTDFMPEDI | |
| | YVEWTNNGKTELNYKNTEPVLDSDGS | |
| | YFMYSKLRVEKKNWVERNSYSCSVVH | |
| | EGLHNHHTTKSFSRTPGK (SEQ ID | |
| | NO: 760) | |
| I23RB31 | QVQLVQSGAEVKKPGSSVKVSCKASG | DIQMTQSPSSLSASVGDRVTITCRASQS |
| 4 | GTFSSYGISWVRQAPGQGLEWMGWIS | ISEDLNWYQQKPGKAPKLLIYAASSLQ |
| | PIFGTANYAQKFQGRVTITADESTSTA | SGVPSRFSGSGSGTDFTLTISSLQPEDF |
| | YMELSSLRSEDTAVYYCARMMMQGF | ATYYCQQSYSTPLTFGQGTKVEIKRAD |
| | DYWGQGTLVTVSSAKTTAPSVYPLAP | AAPTVSIFPPSSEQLTSGGASVVCFLNN |
| | VCGDTTGSSVTLGCLVKGYFPEPVTLT | FYPKDINVKWKIDGSERQNGVLNSWT |
| | WNSGSLSSGVHTFPAVLQSDLYTLSSS | DQDSKDSTYSMSSTLTLTKDEYERHNS |
| | VTVTSSTWPSQSITCNVAHPASSTKVD | YTCEATHKTSTSPIVKSFNRNEC (SEQ |
| | KKIEPRGPTIKPCPPCKCPAPNLLGGPS | ID NO: 763) |
| | VFIFPPKIKDVLMISLSPIVTCVVVDVSE | |
| | DDPDVQISWFVNNVEVHTAQTQTHRE | |
| | DYNSTLRVVSALPIQHQDWMSGKEFK | |
| | CKVNNKDLPAPIERTISKPKGSVRAPQ | |
| | VYVLPPPEEEMTKKQVTLTCMVTDFM | |
| | PEDIYVEWTNNGKTELNYKNTEPVLDS | |
| | DGSYFMYSKLRVEKKNWVERNSYSCS | |
| | VVHEGLHNHHTTKSFSRTPGK (SEQ ID | |
| | NO: 762) | |
| I23RB31 | QVQLVQSGAEVKKPGSSVKVSCKASG | EIVLTQSPATLSLSPGERATLSCRASQS |
| 5 | DTFSSYAISWVRQAPGQGLEWMGWIS | VRKWLAWYQQKPGQAPRLLIYAASN |
| | AIFGNANYAQKFQGRVTITADESTSTA | RATGIPARFSGSGSGTDFTLTISSLEPED |
| | YMELSSLRSEDTAVYYCARGAGWTLL | FAVYYCQQYNNAPLTFGQGTKVEIKR |
| | LDYWGQGTLVTVSSAKTTAPSVYPLA | ADAAPTVSIFPPSSEQLTSGGASVVCFL |
| | PVCGDTTGSSVTLGCLVKGYFPEPVTL | NNFYPKDINVKWKIDGSERQNGVLNS |
| | TWNSGSLSSGVHTFPAVLQSDLYTLSS | WTDQDSKDSTYSMSSTLTLTKDEYER |
| | SVTVTSSTWPSQSITCNVAHPASSTKV | HNSYTCEATHKTSTSPIVKSFNRNEC |
| | DKKIEPRGPTIKPCPPCKCPAPNLLGGP | (SEQ ID NO: 765) |
| | SVFIFPPKIKDVLMISLSPIVTCVVVDVS | |
| | EDDPDVQISWFVNNVEVHTAQTQTHR | |
| | EDYNSTLRVVSALPIQHQDWMSGKEF | |
| | KCKVNNKDLPAPIERTISKPKGSVRAP | |
| | QVYVLPPPEEEMTKKQVTLTCMVTDF | |
| | MPEDIYVEWTNNGKTELNYKNTEPVL | |
| | DSDGSYFMYSKLRVEKKNWVERNSYS | |
| | CSVVHEGLHNHHTTKSFSRTPGK (SEQ | |
| | ID NO: 764) | |
| I23RB31 | QVQLVQSGAEVKKPGSSVKVSCKASG | DIQMTQSPSSLSASVGDRVTITCRASQS |
| 6 | GTFSSYGISWVRQAPGQGLEWMGWIIP | ISNDLNWYQQKPGKAPKLLIYAASSLQ |
| | IFGTTNYAQKFQGRVTITADESTSTAY | SGVPSRFSGSGSGTDFTLTISSLQPEDF |
| | MELSSLRSEDTAVYYCARMMMQSLD | ATYYCQQSYSTPLTFGQGTKVEIKRAD |
| | YWGQGTLVTVSSAKTTAPSVYPLAPV | AAPTVSIFPPSSEQLTSGGASVVCFLNN |
| | CGDTTGSSVTLGCLVKGYFPEPVTLTW | FYPKDINVKWKIDGSERQNGVLNSWT |
| | NSGSLSSGVHTFPAVLQSDLYTLSSSVT | DQDSKDSTYSMSSTLTLTKDEYERHNS |
| | VTSSTWPSQSITCNVAHPASSTKVDKKI | YTCEATHKTSTSPIVKSFNRNEC (SEQ |
| | EPRGPTIKPCPPCKCPAPNLLGGPSVFIF | ID NO: 767) |
| | PPKIKDVLMISLSPIVTCVVVDVSEDDP | |
| | DVQISWFVNNVEVHTAQTQTHREDYN | |
| | STLRVVSALPIQHQDWMSGKEFKCKV | |
| | NNKDLPAPIERTISKPKGSVRAPQVYV | |
| | LPPPEEEMTKKQVTLTCMVTDFMPEDI | |
| | YVEWTNNGKTELNYKNTEPVLDSDGS | |
| | YFMYSKLRVEKKNWVERNSYSCSVVH | |
| | EGLHNHHTTKSFSRTPGK (SEQ ID | |
| | NO: 766) | |
| I23RB31 | QVQLVQSGAEVKKPGSSVKVSCKASG | EIVLTQSPATLSLSPGERATLSCRASQS |
| 7 | GTFSSYAISWVRQAPGQGLEWMGGIIP | VDSFLAWYQQKPGQAPRLLIYTASNR |
| | IFGTANYAQKFQGRVTITADESTSTAY | ATGIPARFSGSGSGTDFTLTISSLEPEDF |
| | MELSSLRSEDTAVYYCARGNGVNYHS | AVYYCQQGNSAPFTFGQGTKVEIKRA |
| | ELDYWGQGTLVTVSSAKTTAPSVYPL | DAAPTVSIFPPSSEQLTSGGASVVCFLN |
| | APVCGDTTGSSVTLGCLVKGYFPEPVT | NFYPKDINVKWKIDGSERQNGVLNSW |
| | LTWNSGSLSSGVHTFPAVLQSDLYTLS | TDQDSKDSTYSMSSTLTLTKDEYERHN |
| | SSVTVTSSTWPSQSITCNVAHPASSTKV | SYTCEATHKTSTSPIVKSFNRNEC (SEQ |
| | DKKIEPRGPTIKPCPPCKCPAPNLLGGP | ID NO: 769) |
| | SVFIFPPKIKDVLMISLSPIVTCVVVDVS | |
| | EDDPDVQISWFVNNVEVHTAQTQTHR | |
| | EDYNSTLRVVSALPIQHQDWMSGKEF | |
| | KCKVNNKDLPAPIERTISKPKGSVRAP | |
| | QVYVLPPPEEEMTKKQVTLTCMVTDF | |
| | MPEDIYVEWTNNGKTELNYKNTEPVL | |
| | DSDGSYFMYSKLRVEKKNWVERNSYS | |
| | CSVVHEGLHNHHTTKSFSRTPGK (SEQ | |
| | ID NO: 768) | |
| I23RB31 | QVQLVQSGAEVKKPGSSVKVSCKASG | DIQMTQSPSSLSASVGDRVTITCRASQS |
| 8 | GTFSSYAISWVRQAPGQGLEWMGGIIP | IDRWLNWYQQKPGKAPKLLIYYASSL |
| | IFGTANYAQKFQGRVTITADESTSTAY | QSGVPSRFSGSGSGTDFTLTISSLQPED |
| | MELSSLRSEDTAVYYCARVRARHRGS | FATYYCQQSYSTPLTFGQGTKVEIKRA |
| | FDYWGQGTLVTVSSAKTTAPSVYPLA | DAAPTVSIFPPSSEQLTSGGASVVCFLN |
| | PVCGDTTGSSVTLGCLVKGYFPEPVTL | NFYPKDINVKWKIDGSERQNGVLNSW |
| | TWNSGSLSSGVHTFPAVLQSDLYTLSS | TDQDSKDSTYSMSSTLTLTKDEYERHN |
| | SVTVTSSTWPSQSITCNVAHPASSTKV | SYTCEATHKTSTSPIVKSFNRNEC (SEQ |
| | DKKIEPRGPTIKPCPPCKCPAPNLLGGP | ID NO: 771) |
| | SVFIFPPKIKDVLMISLSPIVTCVVVDVS | |
| | EDDPDVQISWFVNNVEVHTAQTQTHR | |
| | EDYNSTLRVVSALPIQHQDWMSGKEF | |
| | KCKVNNKDLPAPIERTISKPKGSVRAP | |
| | QVYVLPPPEEEMTKKQVTLTCMVTDF | |
| | MPEDIYVEWTNNGKTELNYKNTEPVL | |
| | DSDGSYFMYSKLRVEKKNWVERNSYS | |
| | CSVVHEGLHNHHTTKSFSRTPGK (SEQ | |
| | ID NO: 770) | |
| I23RB31 | QVQLVQSGAEVKKPGSSVKVSCKASG | DIQMTQSPSSLSASVGDRVTITCRASQS |
| 9 | GTFSSYGISWVRQAPGQGLEWMGWIS | ITDDLNWYQQKPGKAPKLLIYAASSLQ |
| | AIFGTTNYAQKFQGRVTITADESTSTA | SGVPSRFSGSGSGTDFTLTISSLQPEDF |
| | YMELSSLRSEDTALYYCARPLKQAFD | ATYYCQQSYSTPLTFGQGTKVEIKRAD |
| | YWGQGTLVTVSSAKTTAPSVYPLAPV | AAPTVSIFPPSSEQLTSGGASVVCFLNN |
| | CGDTTGSSVTLGCLVKGYFPEPVTLTW | FYPKDINVKWKIDGSERQNGVLNSWT |
| | NSGSLSSGVHTFPAVLQSDLYTLSSSVT | DQDSKDSTYSMSSTLTLTKDEYERHNS |
| | VTSSTWPSQSITCNVAHPASSTKVDKKI | YTCEATHKTSTSPIVKSFNRNEC (SEQ |
| | EPRGPTIKPCPPCKCPAPNLLGGPSVFIF | ID NO: 773) |
| | PPKIKDVLMISLSPIVTCVVVDVSEDDP | |
| | DVQISWFVNNVEVHTAQTQTHREDYN | |
| | STLRVVSALPIQHQDWMSGKEFKCKV | |
| | NNKDLPAPIERTISKPKGSVRAPQVYV | |
| | LPPPEEEMTKKQVTLTCMVTDFMPEDI | |
| | YVEWTNNGKTELNYKNTEPVLDSDGS | |
| | YFMYSKLRVEKKNWVERNSYSCSVVH | |
| | EGLHNHHTTKSFSRTPGK (SEQ ID | |
| | NO: 772) | |
| I23RB32 | QVQLVQSGAEVKKPGSSVKVSCKASG | DIQMTQSPSSLSASVGDRVTITCRASQS |
| 0 | GTFSSYGISWVRQAPGQGLEWMGWIS | ITSDLNWYQQKPGKAPKLLIYAASSLQ |
| | AIFGTTNYAQKFQGRVTITADESTSTA | SGVPSRFSGSGSGTDFTLTISSLQPEDF |
| | YMELSSLRSEDTAVYYCARNGTGNSF | ATYYCQQSYSTPLTFGQGTKVEIKRAD |
| | DYWGQGTLVTVSSAKTTAPSVYPLAP | AAPTVSIFPPSSEQLTSGGASVVCFLNN |
| | VCGDTTGSSVTLGCLVKGYFPEPVTLT | FYPKDINVKWKIDGSERQNGVLNSWT |
| | WNSGSLSSGVHTFPAVLQSDLYTLSSS | DQDSKDSTYSMSSTLTLTKDEYERHNS |
| | VTVTSSTWPSQSITCNVAHPASSTKVD | YTCEATHKTSTSPIVKSFNRNEC (SEQ |
| | KKIEPRGPTIKPCPPCKCPAPNLLGGPS | ID NO: 775) |
| | VFIFPPKIKDVLMISLSPIVTCVVVDVSE | |
| | DDPDVQISWFVNNVEVHTAQTQTHRE | |
| | DYNSTLRVVSALPIQHQDWMSGKEFK | |
| | CKVNNKDLPAPIERTISKPKGSVRAPQ | |
| | VYVLPPPEEEMTKKQVTLTCMVTDFM | |
| | PEDIYVEWTNNGKTELNYKNTEPVLDS | |
| | DGSYFMYSKLRVEKKNWVERNSYSCS | |
| | VVHEGLHNHHTTKSFSRTPGK (SEQ ID | |
| | NO: 774) | |
| I23RB32 | QVQLVQSGAEVKKPGSSVKVSCKASG | DIQMTQSPSSLSASVGDRVTITCRASQS |
| 1 | GTFSSYGISWVRQAPGQGLEWMGWIS | IGGDLNWYQQKPGKAPKLLIYAASSL |
| | AIFGTTNYAQKFQGRVTITADESTSTA | QSGVPSRFSGSGSGTDFTLTISSLQPED |
| | YMELSSLRSEDTAVYYCARHWLQSFD | FATYYCQQSYSTPLTFGQGTKVEIKRA |
| | YWGQGTLVTVSSAKTTAPSVYPLAPV | DAAPTVSIFPPSSEQLTSGGASVVCFLN |
| | CGDTTGSSVTLGCLVKGYFPEPVTLTW | NFYPKDINVKWKIDGSERQNGVLNSW |
| | NSGSLSSGVHTFPAVLQSDLYTLSSSVT | TDQDSKDSTYSMSSTLTLTKDEYERHN |
| | VTSSTWPSQSITCNVAHPASSTKVDKKI | SYTCEATHKTSTSPIVKSFNRNEC (SEQ |
| | EPRGPTIKPCPPCKCPAPNLLGGPSVFIF | ID NO: 777) |
| | PPKIKDVLMISLSPIVTCVVVDVSEDDP | |
| | DVQISWFVNNVEVHTAQTQTHREDYN | |
| | STLRVVSALPIQHQDWMSGKEFKCKV | |
| | NNKDLPAPIERTISKPKGSVRAPQVYV | |
| | LPPPEEEMTKKQVTLTCMVTDFMPEDI | |
| | YVEWTNNGKTELNYKNTEPVLDSDGS | |
| | YFMYSKLRVEKKNWVERNSYSCSVVH | |
| | EGLHNHHTTKSFSRTPGK (SEQ ID | |
| | NO: 776) | |
| I23RB32 | QVQLVQSGAEVKKPGSSVKVSCKASG | EIVLTQSPATLSLSPGERATLSCRASQS |
| 8 | GTFSSYAISWVRQAPGQGLEWMGGIIP | VSSYLAWYQQKPGQAPRLLIYDASNR |
| | IFGTANYAQKFQGRVTITADESTSTAY | ATGVPARFSGSGTGTDFTLTISSLQPED |
| | MELSSLRSEDTAVYYCARGRYIRSYDY | VAVYYCQQRNSTPLTFGQGTKVEIKR |
| | FDYWGQGTLVTVSSAKTTAPSVYPLA | ADAAPTVSIFPPSSEQLTSGGASVVCFL |
| | PVCGDTTGSSVTLGCLVKGYFPEPVTL | NNFYPKDINVKWKIDGSERQNGVLNS |
| | TWNSGSLSSGVHTFPAVLQSDLYTLSS | WTDQDSKDSTYSMSSTLTLTKDEYER |
| | SVTVTSSTWPSQSITCNVAHPASSTKV | HNSYTCEATHKTSTSPIVKSFNRNEC |
| | DKKIEPRGPTIKPCPPCKCPAPNLLGGP | (SEQ ID NO: 779) |
| | SVFIFPPKIKDVLMISLSPIVTCVVVDVS | |
| | EDDPDVQISWFVNNVEVHTAQTQTHR | |
| | EDYNSTLRVVSALPIQHQDWMSGKEF | |
| | KCKVNNKDLPAPIERTISKPKGSVRAP | |
| | QVYVLPPPEEEMTKKQVTLTCMVTDF | |
| | MPEDIYVEWTNNGKTELNYKNTEPVL | |
| | DSDGSYFMYSKLRVEKKNWVERNSYS | |
| | CSVVHEGLHNHHTTKSFSRTPGK (SEQ | |
| | ID NO: 778) | |
| I23RB32 | QVQLVQSGAEVKKPGSSVKVSCKASG | EIVLTQSPATLSLSPGERATLSCRASQS |
| 9 | GTFSSYAISWVRQAPGQGLEWMGGIIP | VSSYLAWYQQKPGQAPRLLIYDASNR |
| | IFGTANYAQKFQGRVTITADESTSTAY | ATGIPARFSGTGSGTDFTLTISSLEPEDF |
| | MELSSLRSEDTAVYYCARDSYYSTVT | ATYYCQQRSNTPLTFGQGTKVEIKRAD |
| | YYYFDYWGQGTLVTVSSAKTTAPSVY | AAPTVSIFPPSSEQLTSGGASVVCFLNN |
| | PLAPVCGDTTGSSVTLGCLVKGYFPEP | FYPKDINVKWKIDGSERQNGVLNSWT |
| | VTLTWNSGSLSSGVHTFPAVLQSDLYT | DQDSKDSTYSMSSTLTLTKDEYERHNS |
| | LSSSVTVTSSTWPSQSITCNVAHPASST | YTCEATHKTSTSPIVKSFNRNEC (SEQ |
| | KVDKKIEPRGPTIKPCPPCKCPAPNLLG | ID NO: 781) |
| | GPSVFIFPPKIKDVLMISLSPIVTCVVVD | |
| | VSEDDPDVQISWFVNNVEVHTAQTQT | |
| | HREDYNSTLRVVSALPIQHQDWMSGK | |
| | EFKCKVNNKDLPAPIERTISKPKGSVRA | |
| | PQVYVLPPPEEEMTKKQVTLTCMVTD | |
| | FMPEDIYVEWTNNGKTELNYKNTEPV | |
| | LDSDGSYFMYSKLRVEKKNWVERNSY | |
| | SCSVVHEGLHNHHTTKSFSRTPGK | |
| | (SEQ ID NO: 780) | |
| I23RB33 | QVQLVQSGAEVKKPGSSVKVSCKASG | EIVLTQSPATLSLSPGERATLSCRASQS |
| 0 | GTFSSYAISWVRQAPGQGLEWMGGIIP | VSSYLAWYQQKPGQAPRLLIYDASNR |
| | IFGTANYAQKFQGRVTITADESTSTAY | ATGIPARFSGSGSGTDFTLTISSLEPEDF |
| | MELSSLRSEDTAVYYCARDRSYSYGLS | AVYYCQQRSNTPLTFGQGTKVEIKRA |
| | GLGYYGFDYWGQGTLVTVSSAKTTAP | DAAPTVSIFPPSSEQLTSGGASVVCFLN |
| | SVYPLAPVCGDTTGSSVTLGCLVKGYF | NFYPKDINVKWKIDGSERQNGVLNSW |
| | PEPVTLTWNSGSLSSGVHTFPAVLQSD | TDQDSKDSTYSMSSTLTLTKDEYERHN |
| | LYTLSSSVTVTSSTWPSQSITCNVAHPA | SYTCEATHKTSTSPIVKSFNRNEC (SEQ |
| | SSTKVDKKIEPRGPTIKPCPPCKCPAPN | ID NO: 783) |
| | LLGGPSVFIFPPKIKDVLMISLSPIVTCV | |
| | VVDVSEDDPDVQISWFVNNVEVHTAQ | |
| | TQTHREDYNSTLRVVSALPIQHQDWM | |
| | SGKEFKCKVNNKDLPAPIERTISKPKGS | |
| | VRAPQVYVLPPPEEEMTKKQVTLTCM | |
| | VTDFMPEDIYVEWTNNGKTELNYKNT | |
| | EPVLDSDGSYFMYSKLRVEKKNWVER | |
| | NSYSCSVVHEGLHNHHTTKSFSRTPGK | |
| | (SEQ ID NO: 782) | |
| I23RB33 | QVQLQQPGAELVMPGASVKLSCKASG | DIVLTQSPASLAVSLGQRATISCKASQS |
| 2 | YTFTSYWMHWVKQRPGQGLEWIGEID | VDYDGDSYMNWYQQKPGQPPKLLIY |
| | PSDSYTNYNQKFKGKSTLTVDKSSSTA | AASNLESGIPARFSGSGSGTDFTLNIHP |
| | YMQLSSLTSEDSAVYYCARSYAFDYW | VEEEDAATYYCQQSNEDPWTFGGGTK |
| | GQGTTVTVSSAKTTAPSVYPLAPVCGD | LEIKRADAAPTVSIFPPSSEQLTSGGAS |
| | TTGSSVTLGCLVKGYFPEPVTLTWNSG | VVCFLNNFYPKDINVKWKIDGSERQN |
| | SLSSGVHTFPAVLQSDLYTLSSSVTVTS | GVLNSWTDQDSKDSTYSMSSTLTLTK |
| | STWPSQSITCNVAHPASSTKVDKKIEPR | DEYERHNSYTCEATHKTSTSPIVKSFN |
| | GPTIKPCPPCKCPAPNLLGGPSVFIFPPK | RNEC (SEQ ID NO: 785) |
| | IKDVLMISLSPIVTCVVVDVSEDDPDV | |
| | QISWFVNNVEVHTAQTQTHREDYNST | |
| | LRVVSALPIQHQDWMSGKEFKCKVNN | |
| | KDLPAPIERTISKPKGSVRAPQVYVLPP | |
| | PEEEMTKKQVTLTCMVTDFMPEDIYV | |
| | EWTNNGKTELNYKNTEPVLDSDGSYF | |
| | MYSKLRVEKKNWVERNSYSCSVVHE | |
| | GLHNHHTTKSFSRTPGK (SEQ ID | |
| | NO: 784) | |

TABLE 8
VH and VL AA Sequences of Examplary Phage Display Based IL-23R antibodies

| Names | VH Sequence | VL Sequence |
|---|---|---|
| I23RB1 | QVQLVQSGAEVKKPGSSVKVSCKASG | EIVLTQSPATLSLSPGERATLSCRASQS |
| | GTFKNYAISWVRQAPGQGLEWMGWI | VSSYLAWYQQKPGQAPRLLIYDASNR |
| | RPIFGTAYYAQKFQGRVTITADESTSTA | ATGIPARFSGSGSGTDFTLTISSLEPEDF |
| | YMELSSLRSEDTAVYYCARDSGSRYA | AVYYCQQRSNWPLTFGQGTKVEIK |
| | GRYAFDYWGQGTLVTVSS (SEQ ID | (SEQ ID NO: 787) |
| | NO: 786) | |
| I23RB2 | QVQLVQSGAEVKKPGSSVKVSCKASG | EIVLTQSPATLSLSPGERATLSCRASQS |
| | GTFSSYAISWVRQAPGQGLEWMGGIIP | VSSYLAWYQQKPGQAPRLLIYDASNR |
| | IFGTANYAQKFQGRVTITADESTSTAY | ATGIPARFSGSGSGTDFTLTISSLEPEDF |
| | MELSSLRSEDTAVYYCARDRYRYLGA | AVYYCQQRSNWPLTFGQGTKVEIK |
| | NGFDYWGQGTLVTVSS (SEQ ID | (SEQ ID NO: 789) |
| | NO: 788) | |
| I23RB3 | QVQLVQSGAEVKKPGSSVKVSCKASG | EIVLTQSPATLSLSPGERATLSCRASQS |
| | GTFKSYSIHWVRQAPGQGLEWMGGIIP | VSSYLAWYQQKPGQAPRLLIYDASNR |
| | YFGYANYAQKFQGRVTITADESTSTAY | ATGIPARFSGSGSGTDFTLTISSLEPEDF |
| | MELSSLRSEDTAVYYCARERGYYGSIY | AVYYCQQRSNWPLTFGQGTKVEIK |
| | YGGGFDYWGQGTLVTVSS (SEQ ID | (SEQ ID NO: 791) |
| | NO: 790) | |
| I23RB4 | QVQLVQSGAEVKKPGSSVKVSCKASG | EIVLTQSPATLSLSPGERATLSCRASQS |
| | GTFKTYAIHWVRQAPGQGLEWMGWII | VSSYLAWYQQKPGQAPRLLIYDASNR |
| | PIFGTANYAQKFQGRVTITADESTSTA | ATGIPARFSGSGSGTDFTLTISSLEPEDF |
| | YMELSSLRSEDTAVYYCARDGGYYGS | AVYYCQQRSNWPLTFGQGTKVEIK |
| | RYYPRSYYFDYWGQGTLVTVSS (SEQ | (SEQ ID NO: 793) |
| | ID NO: 792) | |
| I23RB5 | QVQLVQSGAEVKKPGSSVKVSCKASG | DIQMTQSPSSLSASVGDRVTITCRASQS |
| | GTFSSYAISWVRQAPGQGLEWMGGIIP | ISSYLNWYQQKPGKAPKLLIYAASSLQ |
| | IFGTANYAQKFQGRVTITADESTSTAY | SGVPSRFSGSGSGTDFTLTISSLQPEDF |
| | MELSSLRSEDTAVYYCARDSYYSTVT | ATYYCQQSYSTPLTFGQGTKVEIK |
| | YYYFDYWGQGTLVTVSS (SEQ ID | (SEQ ID NO: 795) |
| | NO: 794) | |
| I23RB6 | QVQLVQSGAEVKKPGSSVKVSCKASG | DIVMTQSPDSLAVSLGERATINCKSSQS |
| | GTFSSYAISWVRQAPGQGLEWMGGIIP | VLYSSNNKNYLAWYQQKPGQPPKLLI |
| | IFGTANYAQKFQGRVTITADESTSTAY | YWASTRESGVPDRFSGSGSGTDFTLTIS |
| | MELSSLRSEDTAVYYCARGRYIRSYDY | SLQAEDVAVYYCQQYYSTPLTFGQGT |
| | FDYWGQGTLVTVSS (SEQ ID NO: 796) | KVEIK (SEQ ID NO: 797) |
| I23RB7 | QIQLVQSGPELKKPGETVKISCKASGY | QIVLTQSPAIMSASPGEKVTMTCSASSS |
| | TFTTYGMSWVKQAPGKGLKWMGWIN | VSYMYWYQQKPGSSPRLLIYDTSNLA |
| | TYSGVPTYADDFKGRFAFSLETSASTA | SGVPVRFSGSGSGTSYSLTISRMEAED |
| | YLQINNLKNEDTATYYCARRDRYGYV | AATYYCQQWSSYPWTFGGGTKLEIK |
| | AGAFAYWGQGTLVTVSS (SEQ ID | (SEQ ID NO: 799) |
| | NO: 798) | |
| I23RB8 | QVQLVQSGAEVKKPGSSVKVSCKASG | EIVLTQSPATLSLSPGERATLSCRASQS |
| | GTFKTDYINWVRQAPGQGLEWMGGII | VSSYLAWYQQKPGQAPRLLIYDASNR |
| | PIFGTANYAQKFQGRVTITADESTSTA | ATGIPARFSGSGSGTDFTLTISSLEPEDF |
| | YMELSSLRSEDTAVYYCARGPSYAYG | AVYYCQQRSNWPLTFGQGTKVEIK |
| | SRTYGYFDYWGQGTLVTVSS (SEQ ID | (SEQ ID NO: 801) |
| | NO: 800) | |
| I23RB9 | QVQLVQSGAEVKKPGSSVKVSCKASG | EIVLTQSPATLSLSPGERATLSCRASQS |
| | GTFSSYAISWVRQAPGQGLEWMGGIIP | VSSYLAWYQQKPGQAPRLLIYDASNR |
| | IFGTANYAQKFQGRVTITADESTSTAY | ATGIPARFSGSGSGTDFTLTISSLEPEDF |
| | MELSSLRSEDTAVYYCARDRSYSYGLS | AVYYCQQRSNWPLTFGQGTKVEIK |
| | GLGYYGFDYWGQGTLVTVSS (SEQ ID | (SEQ ID NO: 803) |
| | NO: 802) | |
| I23RB10 | QVQLVQSGAEVKKPGSSVKVSCKASG | DIVMTQSPDSLAVSLGERATINCKSSQS |
| | GTFSSYAIHWVRQAPGQGLEWMGRIIP | VLYSSNNKNYLAWYQQKPGQPPKLLI |
| | ISGRANYAQKFQGRVTITADESTSTAY | YWASTRESGVPDRFSGSGSGTDFTLTIS |
| | MELSSLRSEDTAVYYCARDSYPSYGSG | SLQAEDVAVYYCQQYYSTPLTFGQGT |
| | GAFGYWGQGTLVTVSS (SEQ ID | KVEIK (SEQ ID NO: 805) |
| | NO: 804) | |
| I23RB259 | QVQLVQSGAEVKKPGSSVKVSCKASG | DIQMTQSPSSLSASVGDRVTITCRASQS |
| | GTFSSYGISWVRQAPGQGLEWMGWIS | ISNDLNWYQQKPGKAPKLLIYAASSLQ |
| | PIFGTTNYAQKFQGRVTITADESTSTAY | SGVPSRFSGSGSGTDFTLTISSLQPEDF |
| | MELSSLRSEDTAVYYCARMMMQSLD | ATYYCQQSYSTPLTFGQGTKVEIK |
| | YWGQGTLVTVSS (SEQ ID NO: 806) | (SEQ ID NO: 807) |
| I23RB260 | QVQLVQSGAEVKKPGSSVKVSCKASG | DIVMTQSPDSLAVSLGERATINCKSSQS |
| | GTFSSYGISWVRQAPGQGLEWMGGIIP | VLYSSNNKNYLAWYQQKPGQPPKLLI |
| | IFGTANYAQKFQGRVTITADESTSTAY | YSASTRESGVPDRFSGSGSGTDFTLTIS |
| | MELSSLRSEDTAVYYCARDFGWSLWF | SLQAEDVAVYYCQQSFHVPWTFGQGT |
| | DYWGQGTLVTVSS (SEQ ID NO: 808) | KVEIK (SEQ ID NO: 809) |
| I23RB261 | QVQLVQSGAEVKKPGSSVKVSCKASG | DIQMTQSPSSLSASVGDRVTITCRASQS |
| | GTFSSYAISWVRQAPGQGLEWMGWIS | INDDLNWYQQKPGKAPKLLIYAASSL |
| | PYFGTANYAQKFQGRVTITADESTSTA | QSGVPSRFSGSGSGTDFTLTISSLQPED |
| | YMELSSLRSEDTAVYYCARMYRQGFD | FATYYCQQSYSTPLTFGQGTKVEIK |
| | YWGQGTLVTVSS (SEQ ID NO: 810) | (SEQ ID NO: 811) |
| I23RB262 | QVQLVQSGAEVKKPGSSVKVSCKASG | DIQMTQSPSSLSASVGDRVTITCRASQS |
| | GTFSSYGISWVRQAPGQGLEWMGWIS | INKDLNWYQQKPGKAPKLLIYFASSLQ |
| | AIFGTANYAQKFQGRVTITADESTSTA | SGVPSRFSGSGSGTDFTLTISSLQPEDF |
| | YMELSSLRSEDTAVYYCARMLKQGFD | ATYYCQQSYSTPLTFGQGTKVEIK |
| | YWGQGTLVTVSS (SEQ ID NO: 812) | (SEQ ID NO: 813) |
| I23RB263 | QVQLVQSGAEVKKPGSSVKVSCKASG | DIQMTQSPSSLSASVGDRVTITCRASQS |
| | GTFSSYAISWVRQAPGQGLEWMGGIIP | ISSYLNWYQQKPGKAPKLLIYAASSLQ |
| | IFGTANYAQKFQGRVTITADESTSTAY | SGVPSRFSGSGSGTDFTLTISSLQPEDF |
| | MELSSLRSEDTAVYYCARFHRFWLDY | ATYYCQQPYSVPFTFGQGTKVEIK |
| | WGQGTLVTVSS (SEQ ID NO: 814) | (SEQ ID NO: 815) |
| I23RB264 | QVQLVQSGAEVKKPGSSVKVSCKASG | DIQMTQSPSSLSASVGDRVTITCRASQS |
| | GTFSSYAISWVRQAPGQGLEWMGGIIP | IDNDLNWYQQKPGKAPKLLIYNASSL |
| | IFGTANYAQKFQGRVTITADESTSTAY | QSGVPSRFSGSGSGTDFTLTISSLQPED |
| | MELSSLRSEDTAVYYCARRSSPGFDY | FATYYCQQPYSIPLTFGQGTKVEIK |
| | WGQGTLVTVSS (SEQ ID NO: 816) | (SEQ ID NO: 817) |
| I23RB265 | QVQLVQSGAEVKKPGSSVKVSCKASG | DIQMTQSPSSLSASVGDRVTITCRASQS |
| | GTFSSYAISWVRQAPGQGLEWMGGIIP | ISTWLNWYQQKPGKAPKLLIYKASSLQ |
| | IFGTANYAQKFQGRVTITADESTSTAY | SGVPSRFSGSGSGTDFTLTISSLQPEDF |
| | MELSSLRSEDTAVYYCARGFVPTQGSL | ATYYCQQYNSPPLTFGQGTKVEIK |
| | DYWGQGTLVTVSS (SEQ ID NO: 818) | (SEQ ID NO: 819) |
| I23RB266 | EVQLLESGGGLVQPGGSLRLSCAASGF | DIQMTQSPSSLSASVGDRVTITCRASQS |
| | TFSDYSMSWVRQAPGKGLEWVSVISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCAKHSMAGNS | ATYYCQQSYSTPLTFGQGTKVEIK |
| | FLRFLDYWGQGTLVTVSS (SEQ ID | (SEQ ID NO: 821) |
| | NO: 820) | |
| I23RB267 | EVQLVQSGAEVKKPGESLKISCKGSGY | EIVLTQSPGTLSLSPGERATLSCRASQS |
| | SFTSYWIGWVRQMPGKGLEWMGIIYP | VSSSYLAWYQQKPGQAPRLLIYGASSR |
| | GDSDTRYSPSFQGQVTISADKSISTAYL | ATGIPDRESGSGSGTDFTLTISRLEPEDF |
| | QWSSLKASDTAMYYCARVWYSSWDL | AVYYCQQYGSSPLTFGQGTKVEIK |
| | DYWGQGTLVTVSS (SEQ ID NO: 822) | (SEQ ID NO: 823) |
| I23RB268 | QVQLVQSGAEVKKPGSSVKVSCKASG | EIVLTQSPATLSLSPGERATLSCRASQS |
| | GTFSSYYIHWVRQAPGQGLEWMGGIIP | VSSYLAWYQQKPGQAPRLLIYDASNR |
| | IFGYANYAQKFQGRVTITADESTSTAY | ATGIPARFSGSGSGTDFTLTISSLEPEDF |
| | MELSSLRSEDTAVYYCARDPGSYGSV | AVYYCQQRSNWPLTFGQGTKVEIK |
| | YYRRSYYFDYWGQGTLVTVSS (SEQ | (SEQ ID NO: 825) |
| | ID NO: 824) | |
| I23RB269 | QVQLVQSGAEVKKPGSSVKVSCKASG | EIVLTQSPGTLSLSPGERATLSCRASQS |
| | GTFSDASISWVRQAPGQGLEWMGTIIPI | VSSSYLAWYQQKPGQAPRLLIYGASSR |
| | SGNANYAQKFQGRVTITADESTSTAY | ATGIPDRFSGSGSGTDFTLTISRLEPEDF |
| | MELSSLRSEDTAVYYCARDRYVWYY | AVYYCQQYGSSPLTFGQGTKVEIK |
| | WDGYFDYWGQGTLVTVSS (SEQ ID | (SEQ ID NO: 827) |
| | NO: 826) | |
| I23RB270 | QVQLVQSGAEVKKPGSSVKVSCKASG | EIVLTQSPATLSLSPGERATLSCRASQS |
| | GTFDSYAISWVRQAPGQGLEWMGGIIP | VSSYLAWYQQKPGQAPRLLIYDASNR |
| | ISGYANYAQKFQGRVTITADESTSTAY | ATGIPARFSGSGSGTDFTLTISSLEPEDF |
| | MELSSLRSEDTAVYYCARDLSGGYLG | AVYYCQQRSNWPLTFGQGTKVEIK |
| | LRTLYYYYFDYWGQGTLVTVSS (SEQ | (SEQ ID NO: 829) |
| | ID NO: 828) | |
| I23RB271 | EVQLLESGGGLVQPGGSLRLSCAASGF | DIQMTQSPSSLSASVGDRVTITCRASQS |
| | TFSSYAMSWVRQAPGKGLEWVSAISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCARDRGSAETF | ATYYCQQSYSTPLTFGQGTKVEIK |
| | DYWGQGTLVTVSS (SEQ ID NO: 830) | (SEQ ID NO: 831) |
| I23RB272 | EVQLLESGGGLVQPGGSLRLSCAASGF | EIVLTQSPGTLSLSPGERATLSCRASQS |
| | TFSSYAMSWVRQAPGKGLEWVSAISG | VSSSYLAWYQQKPGQAPRLLIYGASSR |
| | SGGSTYYADSVKGRFTISRDNSKNTLY | ATGIPDRFSGSGSGTDFTLTISRLEPEDF |
| | LQMNSLRAEDTAVYYCARDFGEPNGG | AVYYCQQYGSSPLTFGQGTKVEIK |
| | FDYWGQGTLVTVSS (SEQ ID NO: 832) | (SEQ ID NO: 833) |
| I23RB273 | EVQLLESGGGLVQPGGSLRLSCAASGF | DIQMTQSPSSLSASVGDRVTITCRASQS |
| | TFSSYAMSWVRQAPGKGLEWVSAISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCARDPSSYYYN | ATYYCQQSYSTPLTFGQGTKVEIK |
| | GYLGSYAFDYWGQGTLVTVSS (SEQ | (SEQ ID NO: 835) |
| | ID NO: 834) | |
| I23RB274 | EVQLLESGGGLVQPGGSLRLSCAASGF | DIQMTQSPSSLSASVGDRVTITCRASQS |
| | TFSSYAMSWVRQAPGKGLEWVSAISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCAREDATDSSD | ATYYCQQSYSTPLTFGQGTKVEIK |
| | YYFDYWGQGTLVTVSS (SEQ ID | (SEQ ID NO: 837) |
| | NO: 836) | |
| I23RB275 | EVQLLESGGGLVQPGGSLRLSCAASGF | DIQMTQSPSSLSASVGDRVTITCRASQS |
| | TFSDSGMSWVRQAPGKGLEWVSAIDG | ISSYLNWYQQKPGKAPKLLIYAASSLQ |
| | SGGTTYYADSVKGRFTISRDNSKNTLY | SGVPSRFSGSGSGTDFTLTISSLQPEDF |
| | LQMNSLRAEDTAVYYCARGSGGYVG | ATYYCQQSYSTPLTFGQGTKVEIK |
| | YGGFDYWGQGTLVTVSS (SEQ ID | (SEQ ID NO: 839) |
| | NO: 838) | |
| I23RB276 | EVQLLESGGGLVQPGGSLRLSCAASGF | DIQMTQSPSSLSASVGDRVTITCRASQS |
| | TFSNYAMSWVRQAPGKGLEWVSAISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCARDGSYGYR | ATYYCQQSYSTPLTFGQGTKVEIK |
| | GRRRRYYDYFDYWGQGTLVTVSS | (SEQ ID NO: 841) |
| | (SEQ ID NO: 840) | |
| I23RB277 | EVQLLESGGGLVQPGGSLRLSCAASGF | DIQMTQSPSSLSASVGDRVTITCRASQS |
| | TFSSYAMSWVRQAPGKGLEWVSAISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCARRRRYRYFD | ATYYCQQSYSTPLTFGQGTKVEIK |
| | YWGQGTLVTVSS (SEQ ID NO: 842) | (SEQ ID NO: 843) |
| I23RB278 | EVQLLESGGGLVQPGGSLRLSCAASGF | EIVLTQSPGTLSLSPGERATLSCRASQS |
| | TFSSYAMSWVRQAPGKGLEWVSAISG | VSSSYLAWYQQKPGQAPRLLIYGASSR |
| | SGGSTYYADSVKGRFTISRDNSKNTLY | ATGIPDRFSGSGSGTDFTLTISRLEPEDF |
| | LQMNSLRAEDTAVYYCVRGGSLGGGF | AVYYCQQYGSSPLTFGQGTKVEIK |
| | YFDYWGQGTLVTVSS (SEQ ID NO: 844) | (SEQ ID NO: 845) |
| I23RB279 | EVQLLESGGGLVQPGGSLRLSCAASGF | DIQMTQSPSSLSASVGDRVTITCRASQS |
| | TFDTYAMHWVRQAPGKGLEWVSWIS | ISSYLNWYQQKPGKAPKLLIYAASSLQ |
| | GEGGSTEYADSVKGRFTISRDNSKNTL | SGVPSRFSGSGSGTDFTLTISSLQPEDF |
| | YLQMNSLRAEDTAVYYCARDLGAYN | ATYYCQQSYSTPLTFGQGTKVEIK |
| | RPFYAFDYWGQGTLVTVSS (SEQ ID | (SEQ ID NO: 847) |
| | NO: 846) | |
| I23RB280 | EVQLLESGGGLVQPGGSLRLSCAASGF | EIVLTQSPGTLSLSPGERATLSCRASQS |
| | TFSNAAMHWVRQAPGKGLEWVSRID | VSSSYLAWYQQKPGQAPRLLIYGASSR |
| | GSGGYTEYADSVKGRFTISRDNSKNTL | ATGIPDRFSGSGSGTDFTLTISRLEPEDF |
| | YLQMNSLRAEDTAVYYCARELYSYLG | AVYYCQQYGSSPLTFGQGTKVEIK |
| | SSYYYGYFDYWGQGTLVTVSS (SEQ | (SEQ ID NO: 849) |
| | ID NO: 848) | |
| I23RB281 | EVQLLESGGGLVQPGGSLRLSCAASGF | DIQMTQSPSSLSASVGDRVTITCRASQS |
| | TFSSYAMSWVRQAPGKGLEWVSAISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCARGPYYSGTS | ATYYCQQSYSTPLTFGQGTKVEIK |
| | GYRSGGGFDYWGQGTLVTVSS (SEQ | (SEQ ID NO: 851) |
| | ID NO: 850) | |
| I23RB282 | EVQLLESGGGLVQPGGSLRLSCAASGF | DIQMTQSPSSLSASVGDRVTITCRASQS |
| | TFSNDAMNWVRQAPGKGLEWVSAID | ISSYLNWYQQKPGKAPKLLIYAASSLQ |
| | GNGGSTNYADSVKGRFTISRDNSKNTL | SGVPSRFSGSGSGTDFTLTISSLQPEDF |
| | YLQMNSLRAEDTAVYYCARDLGGYY | ATYYCQQSYSTPLTFGQGTKVEIK |
| | GRRRRYYGMDVWGQGTLVTVSS | (SEQ ID NO: 853) |
| | (SEQ ID NO: 852) | |
| I23RB283 | EVQLLESGGGLVQPGGSLRLSCAAIGF | EIVLTQSPGTLSLSPGERATLSCRASQS |
| | TFSSSAMHWVRQAPGKGLEWVSAIDG | VSSSYLAWYQQKPGQAPRLLIYGASSR |
| | EGGSTYYADSVKGRFTISRDNSKNTLY | ATGIPDRFSGSGSGTDFTLTISRLEPEDF |
| | LQMNSLRAEDTAVYYCAREVSSYGRY | AVYYCQQYGSSPLTFGQGTKVEIK |
| | GFGYWGQGTLVTVSS (SEQ ID NO: 854) | (SEQ ID NO: 855) |
| I23RB284 | EVQLLESGGGLVQPGGSLRLSCAASGF | DIQMTQSPSSLSASVGDRVTITCRASQS |
| | TFSSYAMSWVRQAPGKGLEWVSAISG | ISSYLNWYQQKPGKAPKLLIYAASSLQ |
| | SGGSTYYADSVKGRFTISRDNSKNTLY | SGVPSRFSGSGSGTDFTLTISSLQPEDF |
| | LQMNSLRAEDTAVYYCARGLAGNSSG | ATYYCQQSYSTPLTFGQGTKVEIK |
| | GGFDYWGQGTLVTVSS (SEQ ID | (SEQ ID NO: 857) |
| | NO: 856) | |
| I23RB285 | EVQLLESGGGLVQPGGSLRLSCAASGF | DIQMTQSPSSLSASVGDRVTITCRASQS |
| | TEDDYAMNWVRQAPGKGLEWVSAIA | ISSYLNWYQQKPGKAPKLLIYAASSLQ |
| | GNGGSTEYADSVKGRFTISRDNSKNTL | SGVPSRFSGSGSGTDFTLTISSLQPEDF |
| | YLQMNSLRAEDTAVYYCARDGGYPLT | ATYYCQQSYSTPLTFGQGTKVEIK |
| | AVTGIYFDYWGQGTLVTVSS (SEQ ID | (SEQ ID NO: 859) |
| | NO: 858) | |
| I23RB286 | EVQLLESGGGLVQPGGSLRLSCAASGF | DIQMTQSPSSLSASVGDRVTITCRASQS |
| | TFSDAGMSWVRQAPGKGLEWVSAISG | ISSYLNWYQQKPGKAPKLLIYAASSLQ |
| | NGGSTNYADSVKGRFTISRDNSKNTLY | SGVPSRFSGSGSGTDFTLTISSLQPEDF |
| | LQMNSLRAEDTAVYYCARRRRRASFD | ATYYCQQSYSTPLTFGQGTKVEIK |
| | YWGQGTLVTVSS (SEQ ID NO: 860) | (SEQ ID NO: 861) |
| I23RB287 | EVQLVQSGAEVKKPGESLKISCKGSGY | EIVLTQSPGTLSLSPGERATLSCRASQS |
| | SFTDAAIHWVRQMPGKGLEWMGTIDP | VSSSYLAWYQQKPGQAPRLLIYGASSR |
| | GDSTTRYSPSFQGQVTISADKSISTAYL | ATGIPDRFSGSGSGTDFTLTISRLEPEDF |
| | QWSSLKASDTAVYYCARDLYYTLEYN | AVYYCQQYGSSPLTFGQGTKVEIK |
| | WYYDAFDYWGQGTLVTVSS (SEQ ID | (SEQ ID NO: 863) |
| | NO: 862) | |
| I23RB288 | EVQLVQSGAEVKKPGESLKISCKGSGY | EIVLTQSPGTLSLSPGERATLSCRASQS |
| | SFKSDWISWVRQMPGKGLEWMGWIY | VSSSYLAWYQQKPGQAPRLLIYGASSR |
| | PGDSRTYYSPSFQGQVTISADKSISTAY | ATGIPDRFSGSGSGTDFTLTISRLEPEDF |
| | LOWSSLKASDTAVYYCARDSSYGTGN | AVYYCQQYGSSPLTFGQGTKVEIK |
| | RGGRSGAFDYWGQGTLVTVSS (SEQ | (SEQ ID NO: 865) |
| | ID NO: 864) | |
| I23RB289 | EVQLVQSGAEVKKPGESLKISCKGSGY | DIQMTQSPSSLSASVGDRVTITCRASQS |
| | SFDSAGINWVRQMPGKGLEWMGNIYP | ISSYLNWYQQKPGKAPKLLIYAASSLQ |
| | GDSDTRYSPSFQGQVTISADKSISTAYL | SGVPSRFSGSGSGTDFTLTISSLQPEDF |
| | QWSSLKASDTAVYYCARGGGSLGFDY | ATYYCQQSYSTPLTFGQGTKVEIK |
| | WGQGTLVTVSS (SEQ ID NO: 866) | (SEQ ID NO: 867) |
| I23RB290 | EVQLVQSGAEVKKPGESLKISCKGSGY | DIQMTQSPSSLSASVGDRVTITCRASQS |
| | SFKNDGIHWVRQMPGKGLEWMGWIA | ISSYLNWYQQKPGKAPKLLIYAASSLQ |
| | PGESTTGYSPSFQGQVTISADKSISTAY | SGVPSRFSGSGSGTDFTLTISSLQPEDF |
| | LOWSSLKASDTAVYYCARDLGGYGSA | ATYYCQQSYSTPLTFGQGTKVEIK |
| | SGRRSDYFDYWGQGTLVTVSS (SEQ | (SEQ ID NO: 869) |
| | ID NO: 868) | |
| I23RB291 | EVQLVQSGAEVKKPGESLKISCKGSGY | EIVLTQSPATLSLSPGERATLSCRASQS |
| | SFTSYYISWVRQMPGKGLEWMGNIYP | VSSYLAWYQQKPGQAPRLLIYDASNR |
| | GDSRTRYSPSFQGQVTISADKSISTAYL | ATGIPARFSGSGSGTDFTLTISSLEPEDF |
| | QWSSLKASDTAVYYCARDRYYGIPFD | AVYYCQQRSNWPLTFGQGTKVEIK |
| | YWGQGTLVTVSS (SEQ ID NO: 870) | (SEQ ID NO: 871) |
| I23RB292 | EVQLVQSGAEVKKPGESLKISCKGSGY | DIQMTQSPSSLSASVGDRVTITCRASQS |
| | SFDTDYINWVRQMPGKGLEWMGYILP | ISSYLNWYQQKPGKAPKLLIYAASSLQ |
| | GESDTRYSPSFQGQVTISADKSISTAYL | SGVPSRFSGSGSGTDFTLTISSLQPEDF |
| | QWSSLKASDTAVYYCARGGGYGLVN | ATYYCQQSYSTPLTFGQGTKVEIK |
| | RRRRSYAFDYWGQGTLVTVSS (SEQ | (SEQ ID NO: 873) |
| | ID NO: 872) | |
| I23RB313 | QVQLVQSGAEVKKPGSSVKVSCKASG | DIQMTQSPSSLSASVGDRVTITCRASQS |
| | GTFSSYGISWVRQAPGQGLEWMGWIS | IGEDLNWYQQKPGKAPKLLIYAASSLQ |
| | AIFGTTNYAQKFQGRVTITADESTSTA | SGVPSRFSGSGSGTDFTLTISSLQPEDF |
| | YMELSSLRSEDTAVYYCARGTVPGFD | ATYYCQQSYSTPLTFGQGTKVEIK |
| | YWGQGTLVTVSS (SEQ ID NO: 874) | (SEQ ID NO: 875) |
| I23RB314 | QVQLVQSGAEVKKPGSSVKVSCKASG | DIQMTQSPSSLSASVGDRVTITCRASQS |
| | GTFSSYGISWVRQAPGQGLEWMGWIS | ISEDLNWYQQKPGKAPKLLIYAASSLQ |
| | PIFGTANYAQKFQGRVTITADESTSTA | SGVPSRFSGSGSGTDFTLTISSLQPEDF |
| | YMELSSLRSEDTAVYYCARMMMQGF | ATYYCQQSYSTPLTFGQGTKVEIK |
| | DYWGQGTLVTVSS (SEQ ID NO: 876) | (SEQ ID NO: 877) |
| I23RB315 | QVQLVQSGAEVKKPGSSVKVSCKASG | EIVLTQSPATLSLSPGERATLSCRASQS |
| | DTFSSYAISWVRQAPGQGLEWMGWIS | VRKWLAWYQQKPGQAPRLLIYAASN |
| | AIFGNANYAQKFQGRVTITADESTSTA | RATGIPARFSGSGSGTDFTLTISSLEPED |
| | YMELSSLRSEDTAVYYCARGAGWTLL | FAVYYCQQYNNAPLTFGQGTKVEIK |
| | LDYWGQGTLVTVSS (SEQ ID NO: 878) | (SEQ ID NO: 879) |
| I23RB316 | QVQLVQSGAEVKKPGSSVKVSCKASG | DIQMTQSPSSLSASVGDRVTITCRASQS |
| | GTFSSYGISWVRQAPGQGLEWMGWIIP | ISNDLNWYQQKPGKAPKLLIYAASSLQ |
| | IFGTTNYAQKFQGRVTITADESTSTAY | SGVPSRFSGSGSGTDFTLTISSLQPEDF |
| | MELSSLRSEDTAVYYCARMMMQSLD | ATYYCQQSYSTPLTFGQGTKVEIK |
| | YWGQGTLVTVSS (SEQ ID NO: 880) | (SEQ ID NO: 881) |
| I23RB317 | QVQLVQSGAEVKKPGSSVKVSCKASG | EIVLTQSPATLSLSPGERATLSCRASQS |
| | GTFSSYAISWVRQAPGQGLEWMGGIIP | VDSFLAWYQQKPGQAPRLLIYTASNR |
| | IFGTANYAQKFQGRVTITADESTSTAY | ATGIPARFSGSGSGTDFTLTISSLEPEDF |
| | MELSSLRSEDTAVYYCARGNGVNYHS | AVYYCQQGNSAPFTFGQGTKVEIK |
| | ELDYWGQGTLVTVSS (SEQ ID NO: 882) | (SEQ ID NO: 883) |
| I23RB318 | QVQLVQSGAEVKKPGSSVKVSCKASG | DIQMTQSPSSLSASVGDRVTITCRASQS |
| | GTFSSYAISWVRQAPGQGLEWMGGIIP | IDRWLNWYQQKPGKAPKLLIYYASSL |
| | IFGTANYAQKFQGRVTITADESTSTAY | QSGVPSRFSGSGSGTDFTLTISSLQPED |
| | MELSSLRSEDTAVYYCARVRARHRGS | FATYYCQQSYSTPLTFGQGTKVEIK |
| | FDYWGQGTLVTVSS (SEQ ID NO: 884) | (SEQ ID NO: 885) |
| I23RB319 | QVQLVQSGAEVKKPGSSVKVSCKASG | DIQMTQSPSSLSASVGDRVTITCRASQS |
| | GTFSSYGISWVRQAPGQGLEWMGWIS | ITDDLNWYQQKPGKAPKLLIYAASSLQ |
| | AIFGTTNYAQKFQGRVTITADESTSTA | SGVPSRFSGSGSGTDFTLTISSLQPEDF |
| | YMELSSLRSEDTALYYCARPLKQAFD | ATYYCQQSYSTPLTFGQGTKVEIK |
| | YWGQGTLVTVSS (SEQ ID NO: 886) | (SEQ ID NO: 887) |
| I23RB320 | QVQLVQSGAEVKKPGSSVKVSCKASG | DIQMTQSPSSLSASVGDRVTITCRASQS |
| | GTFSSYGISWVRQAPGQGLEWMGWIS | ITSDLNWYQQKPGKAPKLLIYAASSLQ |
| | AIFGTTNYAQKFQGRVTITADESTSTA | SGVPSRFSGSGSGTDFTLTISSLQPEDF |
| | YMELSSLRSEDTAVYYCARNGTGNSF | ATYYCQQSYSTPLTFGQGTKVEIK |
| | DYWGQGTLVTVSS (SEQ ID NO: 888) | (SEQ ID NO: 889) |
| I23RB321 | QVQLVQSGAEVKKPGSSVKVSCKASG | DIQMTQSPSSLSASVGDRVTITCRASQS |
| | GTFSSYGISWVRQAPGQGLEWMGWIS | IGGDLNWYQQKPGKAPKLLIYAASSL |
| | AIFGTTNYAQKFQGRVTITADESTSTA | QSGVPSRFSGSGSGTDFTLTISSLQPED |
| | YMELSSLRSEDTAVYYCARHWLQSFD | FATYYCQQSYSTPLTFGQGTKVEIK |
| | YWGQGTLVTVSS (SEQ ID NO: 890) | (SEQ ID NO: 891) |
| I23RB328 | QVQLVQSGAEVKKPGSSVKVSCKASG | EIVLTQSPATLSLSPGERATLSCRASQS |
| | GTFSSYAISWVRQAPGQGLEWMGGIIP | VSSYLAWYQQKPGQAPRLLIYDASNR |
| | IFGTANYAQKFQGRVTITADESTSTAY | ATGVPARFSGSGTGTDFTLTISSLQPED |
| | MELSSLRSEDTAVYYCARGRYIRSYDY | VAVYYCQQRNSTPLTFGQGTKVEIK |
| | FDYWGQGTLVTVSS (SEQ ID NO: 892) | (SEQ ID NO: 893) |
| I23RB329 | QVQLVQSGAEVKKPGSSVKVSCKASG | EIVLTQSPATLSLSPGERATLSCRASQS |
| | GTFSSYAISWVRQAPGQGLEWMGGIIP | VSSYLAWYQQKPGQAPRLLIYDASNR |
| | IFGTANYAQKFQGRVTITADESTSTAY | ATGIPARFSGTGSGTDFTLTISSLEPEDF |
| | MELSSLRSEDTAVYYCARDSYYSTVT | ATYYCQQRSNTPLTFGQGTKVEIK |
| | YYYFDYWGQGTLVTVSS (SEQ ID | (SEQ ID NO: 895) |
| | NO: 894) | |
| I23RB330 | QVQLVQSGAEVKKPGSSVKVSCKASG | EIVLTQSPATLSLSPGERATLSCRASQS |
| | GTFSSYAISWVRQAPGQGLEWMGGIIP | VSSYLAWYQQKPGQAPRLLIYDASNR |
| | IFGTANYAQKFQGRVTITADESTSTAY | ATGIPARFSGSGSGTDFTLTISSLEPEDF |
| | MELSSLRSEDTAVYYCARDRSYSYGLS | AVYYCQQRSNTPLTFGQGTKVEIK |
| | GLGYYGFDYWGQGTLVTVSS (SEQ ID | (SEQ ID NO: 897) |
| | NO: 896) | |
| I23RB332 | QVQLQQPGAELVMPGASVKLSCKASG | DIVLTQSPASLAVSLGQRATISCKASQS |
| | YTFTSYWMHWVKQRPGQGLEWIGEID | VDYDGDSYMNWYQQKPGQPPKLLIY |
| | PSDSYTNYNQKFKGKSTLTVDKSSSTA | AASNLESGIPARFSGSGSGTDFTLNIHP |
| | YMQLSSLTSEDSAVYYCARSYAFDYW | VEEEDAATYYCQQSNEDPWTFGGGTK |
| | GQGTTVTVSS (SEQ ID NO: 898) | LEIK (SEQ ID NO: 899) |

TABLE 9
CDR AA Sequences of Exemplary Phage Display Based IL-23R antibodies

| Names | HCDR1 | HCDR2 | HCDR3 | LCDR1 | LCDR2 | LCDR3 |
|---|---|---|---|---|---|---|
| I23RB1 | GGTFKNY | IRPIFGTA | ARDSGSR | QSVSSY | DAS (SEQ | QQRSNWP |
| | A (SEQ ID | (SEQ ID | YAGRYAF | (SEQ ID | ID NO: 904) | LT (SEQ ID |
| | NO: 900) | NO: 901) | DY (SEQ ID | NO: 903) | | NO: 905) |
| | | | NO: 902) | | | |
| I23RB2 | GGTFSSYA | IIPIFGTA | ARDRYRY | QSVSSY | DAS (SEQ | QQRSNWP |
| | (SEQ ID | (SEQ ID | LGANGFD | (SEQ ID | ID NO: 910) | LT (SEQ ID |
| | NO: 906) | NO: 907) | Y (SEQ ID | NO: 909) | | NO: 911) |
| | | | NO: 908) | | | |
| I23RB3 | GGTFKSYS | IIPYFGYA | ARERGYY | QSVSSY | DAS (SEQ | QQRSNWP |
| | (SEQ ID | (SEQ ID | GSIYYGGG | (SEQ ID | ID NO: 916) | LT (SEQ ID |
| | NO: 912) | NO: 913) | FDY (SEQ | NO: 915) | | NO: 917) |
| | | | ID NO: 914) | | | |
| I23RB4 | GGTFKTY | IIPIFGTA | ARDGGYY | QSVSSY | DAS (SEQ | QQRSNWP |
| | A (SEQ ID | (SEQ ID | GSRYYPRS | (SEQ ID | ID NO: 922) | LT (SEQ ID |
| | NO: 918) | NO: 919) | YYFDY | NO: 921) | | NO: 923) |
| | | | (SEQ ID | | | |
| | | | NO: 920) | | | |
| I23RB5 | GGTFSSYA | IIPIFGTA | ARDSYYST | QSISSY | AAS (SEQ | QQSYSTPL |
| | (SEQ ID | (SEQ ID | VTYYYFD | (SEQ ID | ID NO: 928) | T (SEQ ID |
| | NO: 924) | NO: 925) | Y (SEQ ID | NO: 927) | | NO: 929) |
| | | | NO: 926) | | | |
| I23RB6 | GGTFSSYA | IIPIFGTA | ARGRYIRS | QSVLYSSN | WAS (SEQ | QQYYSTPL |
| | (SEQ ID | (SEQ ID | YDYFDY | NKNY | ID NO: 934) | T (SEQ ID |
| | NO: 930) | NO: 931) | (SEQ ID | (SEQ ID | | NO: 935) |
| | | | NO: 932) | NO: 933) | | |
| I23RB7 | GYTFTTYG | INTYSGVP | ARRDRYG | SSVSY | DTS (SEQ | QQWSSYP |
| | (SEQ ID | (SEQ ID | YVAGAFA | (SEQ ID | ID NO: 940) | WT (SEQ |
| | NO: 936) | NO: 937) | Y (SEQ ID | NO: 939) | | ID NO: 941) |
| | | | NO: 938) | | | |
| I23RB8 | GGTFKTD | IIPIFGTA | ARGPSYA | QSVSSY | DAS (SEQ | QQRSNWP |
| | Y (SEQ ID | (SEQ ID | YGSRTYG | (SEQ ID | ID NO: 946) | LT (SEQ ID |
| | NO: 942) | NO: 943) | YFDY (SEQ | NO: 945) | | NO: 947) |
| | | | ID NO: 944) | | | |
| I23RB9 | GGTFSSYA | IIPIFGTA | ARDRSYS | QSVSSY | DAS (SEQ | QQRSNWP |
| | (SEQ ID | (SEQ ID | YGLSGLG | (SEQ ID | ID NO: 952) | LT (SEQ ID |
| | NO: 948) | NO: 949) | YYGFDY | NO: 951) | | NO: 953) |
| | | | (SEQ ID | | | |
| | | | NO: 950) | | | |
| I23RB10 | GGTFSSYA | IIPISGRA | ARDSYPSY | QSVLYSSN | WAS (SEQ | QQYYSTPL |
| | (SEQ ID | (SEQ ID | GSGGAFG | NKNY | ID NO: 958) | T (SEQ ID |
| | NO: 954) | NO: 955) | Y (SEQ ID | (SEQ ID | | NO: 959) |
| | | | NO: 956) | NO: 957) | | |
| I23RB259 | GGTFSSYG | ISPIFGTT | ARMMMQ | QSISND | AAS (SEQ | QQSYSTPL |
| | (SEQ ID | (SEQ ID | SLDY (SEQ | (SEQ ID | ID NO: 964) | T (SEQ ID |
| | NO: 960) | NO: 961) | ID NO: 962) | NO: 963) | | NO: 965) |
| I23RB260 | GGTFSSYG | IIPIFGTA | ARDFGWS | QSVLYSSN | SAS (SEQ | QQSFHVP |
| | (SEQ ID | (SEQ ID | LWFDY | NKNY | ID NO: 970) | WT (SEQ |
| | NO: 966) | NO: 967) | (SEQ ID | (SEQ ID | | ID NO: 971) |
| | | | NO: 968) | NO: 969) | | |
| I23RB261 | GGTFSSYA | ISPYFGTA | ARMYRQG | QSINDD | AAS (SEQ | QQSYSTPL |
| | (SEQ ID | (SEQ ID | FDY (SEQ | (SEQ ID | ID NO: 976) | T (SEQ ID |
| | NO: 972) | NO: 973) | ID NO: 974) | NO: 975) | | NO: 977) |
| I23RB262 | GGTFSSYG | ISAIFGTA | ARMLKQG | QSINKD | FAS (SEQ | QQSYSTPL |
| | (SEQ | (SEQ ID | FDY (SEQ | (SEQ ID | ID NO: 982) | T (SEQ ID |
| | ID NO: 978) | NO: 979) | ID NO: 980) | NO: 981) | | NO: 983) |
| I23RB263 | GGTFSSYA | IIPIFGTA | ARFHRFW | QSISSY | AAS (SEQ | QQPYSVPF |
| | (SEQ ID | (SEQ ID | LDY (SEQ | (SEQ ID | ID NO: 988) | T (SEQ ID |
| | NO: 984) | NO: 985) | ID NO: 986) | NO: 987) | | NO: 989) |
| I23RB264 | GGTFSSYA | IIPIFGTA | ARRSSPGF | QSIDND | NAS (SEQ | QQPYSIPL |
| | (SEQ ID | (SEQ ID | DY (SEQ ID | (SEQ ID | ID NO: 994) | T (SEQ ID |
| | NO: 990) | NO: 991) | NO: 992) | NO: 993) | | NO: 995) |
| I23RB265 | GGTFSSYA | IIPIFGTA | ARGFVPTQ | QSISTW | KAS (SEQ | QQYNSPPL |
| | (SEQ ID | (SEQ ID | GSLDY | (SEQ ID | ID | T (SEQ ID |
| | NO: 996) | NO: 997) | (SEQ ID | NO: 999) | NO: 1000) | NO: 1001) |
| | | | NO: 998) | | | |
| I23RB266 | GFTFSDYS | ISGSGGST | AKHSMAG | QSISSY | AAS (SEQ | QQSYSTPL |
| | (SEQ ID | (SEQ ID | NSFLRFLD | (SEQ ID | ID | T (SEQ ID |
| | NO: 1002) | NO: 1003) | Y (SEQ ID | NO: 1005) | NO: 1006) | NO: 1007) |
| | | | NO: 1004) | | | |
| I23RB267 | GYSFTSY | IYPGDSDT | ARVWYSS | QSVSSSY | GAS (SEQ | QQYGSSPL |
| | W (SEQ ID | (SEQ ID | WDLDY | (SEQ ID | ID | T (SEQ ID |
| | NO: 1008) | NO: 1009) | (SEQ ID | NO: 1011) | NO: 1012) | NO: 1013) |
| | | | NO: 1010) | | | |
| I23RB268 | GGTFSSYY | IIPIFGYA | ARDPGSY | QSVSSY | DAS (SEQ | QQRSNWP |
| | (SEQ ID | (SEQ ID | GSVYYRR | (SEQ ID | ID | LT (SEQ ID |
| | NO: 1014) | NO: 1015) | SYYFDY | NO: 1017) | NO: 1018) | NO: 1019) |
| | | | (SEQ ID | | | |
| | | | NO: 1016) | | | |
| I23RB269 | GGTFSDAS | IIPISGNA | ARDRYVW | QSVSSSY | GAS (SEQ | QQYGSSPL |
| | (SEQ ID | (SEQ ID | YYWDGYF | (SEQ ID | ID | T (SEQ ID |
| | NO: 1020) | NO: 1021) | DY (SEQ ID | NO: 1023) | NO: 1024) | NO: 1025) |
| | | | NO: 1022) | | | |
| I23RB270 | GGTFDSY | IIPISGYA | ARDLSGG | QSVSSY | DAS (SEQ | QQRSNWP |
| | A (SEQ ID | (SEQ ID | YLGLRTLY | (SEQ ID | ID | LT (SEQ ID |
| | NO: 1026) | NO: 1027) | YYYFDY | NO: 1029) | NO: 1030) | NO: 1031) |
| | | | (SEQ ID | | | |
| | | | NO: 1028) | | | |
| I23RB271 | GFTFSSYA | ISGSGGST | ARDRGSA | QSISSY | AAS (SEQ | QQSYSTPL |
| | (SEQ ID | (SEQ ID | ETFDY | (SEQ ID | ID | T (SEQ ID |
| | NO: 1032) | NO: 1033) | (SEQ ID | NO: 1035) | NO: 1036) | NO: 1037) |
| | | | NO: 1034) | | | |
| I23RB272 | GFTFSSYA | ISGSGGST | ARDFGEPN | QSVSSSY | GAS (SEQ | QQYGSSPL |
| | (SEQ ID | (SEQ ID | GGFDY | (SEQ ID | ID | T (SEQ ID |
| | NO: 1038) | NO: 1039) | (SEQ ID | NO: 1041) | NO: 1042) | NO: 1043) |
| | | | NO: 1040) | | | |
| I23RB273 | GFTFSSYA | ISGSGGST | ARDPSSYY | QSISSY | AAS (SEQ | QQSYSTPL |
| | (SEQ ID | (SEQ ID | YNGYLGS | (SEQ ID | ID | T (SEQ ID |
| | NO: 1044) | NO: 1045) | YAFDY | NO: 1047) | NO: 1048) | NO: 1049) |
| | | | (SEQ ID | | | |
| | | | NO: 1046) | | | |
| I23RB274 | GFTFSSYA | ISGSGGST | AREDATD | QSISSY | AAS (SEQ | QQSYSTPL |
| | (SEQ ID | (SEQ ID | SSDYYFDY | (SEQ ID | ID | T (SEQ ID |
| | NO: 1050) | NO: 1051) | (SEQ ID | NO: 1053) | NO: 1054) | NO: 1055) |
| | | | NO: 1052) | | | |
| I23RB275 | GFTFSDSG | IDGSGGTT | ARGSGGY | QSISSY | AAS (SEQ | QQSYSTPL |
| | (SEQ ID | (SEQ ID | VGYGGFD | (SEQ ID | ID | T (SEQ ID |
| | NO: 1056) | NO: 1057) | Y (SEQ ID | NO: 1059) | NO: 1060) | NO: 1061) |
| | | | NO: 1058) | | | |
| I23RB276 | GFTFSNYA | ISGSGGST | ARDGSYG | QSISSY | AAS (SEQ | QQSYSTPL |
| | (SEQ ID | (SEQ ID | YRGRRRR | (SEQ ID | ID | T (SEQ ID |
| | NO: 1062) | NO: 1063) | YYDYFDY | NO: 1065) | NO: 1066) | NO: 1067) |
| | | | (SEQ ID | | | |
| | | | NO: 1064) | | | |
| I23RB277 | GFTFSSYA | ISGSGGST | ARRRRYR | QSISSY | AAS (SEQ | QQSYSTPL |
| | (SEQ ID | (SEQ ID | YFDY (SEQ | (SEQ ID | ID | T (SEQ ID |
| | NO: 1068) | NO: 1069) | ID | NO: 1071) | NO: 1072) | NO: 1073) |
| | | | NO: 1070) | | | |
| I23RB278 | GFTFSSYA | ISGSGGST | VRGGSLG | QSVSSSY | GAS (SEQ | QQYGSSPL |
| | (SEQ ID | (SEQ ID | GGFYFDY | (SEQ ID | ID | T (SEQ ID |
| | NO: 1074) | NO: 1075) | (SEQ ID | NO: 1077) | NO: 1078) | NO: 1079) |
| | | | NO: 1076) | | | |
| I23RB279 | GFTFDTYA | ISGEGGST | ARDLGAY | QSISSY | AAS (SEQ | QQSYSTPL |
| | (SEQ ID | (SEQ ID | NRPFYAFD | (SEQ ID | ID | T (SEQ ID |
| | NO: 1080) | NO: 1081) | Y (SEQ ID | NO: 1083) | NO: 1084) | NO: 1085) |
| | | | NO: 1082) | | | |
| I23RB280 | GFTFSNAA | IDGSGGYT | ARELYSYL | QSVSSSY | GAS (SEQ | QQYGSSPL |
| | (SEQ ID | (SEQ ID | GSSYYYG | (SEQ ID | ID | T (SEQ ID |
| | NO: 1086) | NO: 1087) | YFDY (SEQ | NO: 1089) | NO: 1090) | NO: 1091) |
| | | | ID | | | |
| | | | NO: 1088) | | | |
| I23RB281 | GFTFSSYA | ISGSGGST | ARGPYYS | QSISSY | AAS (SEQ | QQSYSTPL |
| | (SEQ ID | (SEQ ID | GTSGYRSG | (SEQ ID | ID | T (SEQ ID |
| | NO: 1092) | NO: 1093) | GGFDY | NO: 1095) | NO: 1096) | NO: 1097) |
| | | | (SEQ ID | | | |
| | | | NO: 1094) | | | |
| I23RB282 | GFTFSNDA | IDGNGGST | ARDLGGY | QSISSY | AAS (SEQ | QQSYSTPL |
| | (SEQ ID | (SEQ ID | YGRRRRY | (SEQ ID | ID | T (SEQ ID |
| | NO: 1098) | NO: 1099) | YGMDV | NO: 1101) | NO: 1102) | NO: 1103) |
| | | | (SEQ ID | | | |
| | | | NO: 1100) | | | |
| I23RB283 | GFTFSSSA | IDGEGGST | AREVSSYG | QSVSSSY | GAS (SEQ | QQYGSSPL |
| | (SEQ ID | (SEQ ID | RYGFGY | (SEQ ID | ID | T (SEQ ID |
| | NO: 1104) | NO: 1105) | (SEQ ID | NO: 1107) | NO: 1108) | NO: 1109) |
| | | | NO: 1106) | | | |
| I23RB284 | GFTFSSYA | ISGSGGST | ARGLAGN | QSISSY | AAS (SEQ | QQSYSTPL |
| | (SEQ ID | (SEQ ID | SSGGGFDY | (SEQ ID | ID | T (SEQ ID |
| | NO: 1110) | NO: 1111) | (SEQ ID | NO: 1113) | NO: 1114) | NO: 1115) |
| | | | NO: 1112) | | | |
| I23RB285 | GFTFDDY | IAGNGGST | ARDGGYP | QSISSY | AAS (SEQ | QQSYSTPL |
| | A (SEQ ID | (SEQ ID | LTAVTGIY | (SEQ ID | ID | T (SEQ ID |
| | NO: 1116) | NO: 1117) | FDY (SEQ | NO: 1119) | NO: 1120) | NO: 1121) |
| | | | ID | | | |
| | | | NO: 1118) | | | |
| I23RB286 | GFTFSDAG | ISGNGGST | ARRRRRA | QSISSY | AAS (SEQ | QQSYSTPL |
| | (SEQ ID | (SEQ ID | SFDY (SEQ | (SEQ ID | ID | T (SEQ ID |
| | NO: 1122) | NO: 1123) | ID | NO: 1125) | NO: 1126) | NO: 1127) |
| | | | NO: 1124) | | | |
| I23RB287 | GYSFTDA | IDPGDSTT | ARDLYYT | QSVSSSY | GAS (SEQ | QQYGSSPL |
| | A (SEQ ID | (SEQ ID | LEYNWYY | (SEQ ID | ID | T (SEQ ID |
| | NO: 1128) | NO: 1129) | DAFDY | NO: 1131) | NO: 1132) | NO: 1133) |
| | | | (SEQ ID | | | |
| | | | NO: 1130) | | | |
| I23RB288 | GYSFKSD | IYPGDSRT | ARDSSYGT | QSVSSSY | GAS (SEQ | QQYGSSPL |
| | W (SEQ ID | (SEQ ID | GNRGGRS | (SEQ ID | ID | T (SEQ ID |
| | NO: 1134) | NO: 1135) | GAFDY | NO: 1137) | NO: 1138) | NO: 1139) |
| | | | (SEQ ID | | | |
| | | | NO: 1136) | | | |
| I23RB289 | GYSFDSAG | IYPGDSDT | ARGGGSL | QSISSY | AAS (SEQ | QQSYSTPL |
| | (SEQ ID | (SEQ ID | GFDY (SEQ | (SEQ ID | ID | T (SEQ ID |
| | NO: 1140) | NO: 1141) | ID | NO: 1143) | NO: 1144) | NO: 1145) |
| | | | NO: 1142) | | | |
| I23RB290 | GYSFKND | IAPGESTT | ARDLGGY | QSISSY | AAS (SEQ | QQSYSTPL |
| | G (SEQ ID | (SEQ ID | GSASGRRS | (SEQ ID | ID | T (SEQ ID |
| | NO: 1146) | NO: 1147) | DYFDY | NO: 1149) | NO: 1150) | NO: 1151) |
| | | | (SEQ ID | | | |
| | | | NO: 1148) | | | |
| I23RB291 | GYSFTSYY | IYPGDSRT | ARDRYYGI | QSVSSY | DAS (SEQ | QQRSNWP |
| | (SEQ ID | (SEQ ID | PFDY (SEQ | (SEQ ID | ID | LT (SEQ ID |
| | NO: 1152) | NO: 1153) | ID | NO: 1155) | NO: 1156) | NO: 1157) |
| | | | NO: 1154) | | | |
| I23RB292 | GYSFDTD | ILPGESDT | ARGGGYG | QSISSY | AAS (SEQ | QQSYSTPL |
| | Y (SEQ ID | (SEQ ID | LVNRRRRS | (SEQ ID | ID | T (SEQ ID |
| | NO: 1158) | NO: 1159) | YAFDY | NO: 1161) | NO: 1162) | NO: 1163) |
| | | | (SEQ ID | | | |
| | | | NO: 1160) | | | |
| I23RB313 | GGTFSSYG | ISAIFGTT | ARGTVPGF | QSIGED | AAS (SEQ | QQSYSTPL |
| | (SEQ ID | (SEQ ID | DY (SEQ ID | (SEQ ID | ID | T (SEQ ID |
| | NO: 1164) | NO: 1165) | NO: 1166) | NO: 1167) | NO: 1168) | NO: 1169) |
| I23RB314 | GGTFSSYG | ISPIFGTA | ARMMMQ | QSISED | AAS (SEQ | QQSYSTPL |
| | (SEQ ID | (SEQ ID | GFDY (SEQ | (SEQ ID | ID | T (SEQ ID |
| | NO: 1170) | NO: 1171) | ID | NO: 1173) | NO: 1174) | NO: 1175) |
| | | | NO: 1172) | | | |
| I23RB315 | GDTFSSYA | ISAIFGNA | ARGAGWT | QSVRKW | AAS (SEQ | QQYNNAP |
| | (SEQ ID | (SEQ ID | LLLDY | (SEQ ID | ID | LT (SEQ ID |
| | NO: 1176) | NO: 1177) | (SEQ ID | NO: 1179) | NO: 1180) | NO: 1181) |
| | | | NO: 1178) | | | |
| I23RB316 | GGTFSSYG | IIPIFGTT | ARMMMQ | QSISND | AAS (SEQ | QQSYSTPL |
| | (SEQ ID | (SEQ ID | SLDY (SEQ | (SEQ ID | ID | T (SEQ ID |
| | NO: 1182) | NO: 1183) | ID | NO: 1185) | NO: 1186) | NO: 1187) |
| | | | NO: 1184) | | | |
| I23RB317 | GGTFSSYA | IIPIFGTA | ARGNGVN | QSVDSF | TAS (SEQ | QQGNSAPF |
| | (SEQ ID | (SEQ ID | YHSELDY | (SEQ ID | ID | T (SEQ ID |
| | NO: 1188) | NO: 1189) | (SEQ ID | NO: 1191) | NO: 1192) | NO: 1193) |
| | | | NO: 1190) | | | |
| I23RB318 | GGTFSSYA | IIPIFGTA | ARVRARH | QSIDRW | YAS (SEQ | QQSYSTPL |
| | (SEQ ID | (SEQ ID | RGSFDY | (SEQ ID | ID | T (SEQ ID |
| | NO: 1194) | NO: 1195) | (SEQ ID | NO: 1197) | NO: 1198) | NO: 1199) |
| | | | NO: 1196) | | | |
| I23RB319 | GGTFSSYG | ISAIFGTT | ARPLKQAF | QSITDD | AAS (SEQ | QQSYSTPL |
| | (SEQ ID | (SEQ ID | DY (SEQ ID | (SEQ ID | ID | T (SEQ ID |
| | NO: 1200) | NO: 1201) | NO: 1202) | NO: 1203) | NO: 1204) | NO: 1205) |
| I23RB320 | GGTFSSYG | ISAIFGTT | ARNGTGN | QSITSD | AAS (SEQ | QQSYSTPL |
| | (SEQ ID | (SEQ ID | SFDY (SEQ | (SEQ ID | ID | T (SEQ ID |
| | NO: 1206) | NO: 1207) | ID | NO: 1209) | NO: 1210) | NO: 1211) |
| | | | NO: 1208) | | | |
| I23RB321 | GGTFSSYG | ISAIFGTT | ARHWLQS | QSIGGD | AAS (SEQ | QQSYSTPL |
| | (SEQ ID | (SEQ ID | FDY (SEQ | (SEQ ID | ID | T (SEQ ID |
| | NO: 1212) | NO: 1213) | ID | NO: 1215) | NO: 1216) | NO: 1217) |
| | | | NO: 1214) | | | |
| I23RB328 | GGTFSSYA | IIPIFGTA | ARGRYIRS | QSVSSY | DAS (SEQ | QQRNSTPL |
| | (SEQ ID | (SEQ ID | YDYFDY | (SEQ ID | ID | T (SEQ ID |
| | NO: 1218) | NO: 1219) | (SEQ ID | NO: 1221) | NO: 1222) | NO: 1223) |
| | | | NO: 1220) | | | |
| I23RB329 | GGTFSSYA | IIPIFGTA | ARDSYYST | QSVSSY | DAS (SEQ | QQRSNTPL |
| | (SEQ ID | (SEQ ID | VTYYYFD | (SEQ ID | ID | T (SEQ ID |
| | NO: 1224) | NO: 1225) | Y (SEQ ID | NO: 1227) | NO: 1228) | NO: 1229) |
| | | | NO: 1226) | | | |
| I23RB330 | GGTFSSYA | IIPIFGTA | ARDRSYS | QSVSSY | DAS (SEQ | QQRSNTPL |
| | (SEQ ID | (SEQ ID | YGLSGLG | (SEQ ID | ID | T (SEQ ID |
| | | NO: 1231) | YYGFDY | NO: 1233) | NO: 1234) | NO: 1235) |
| | | | (SEQ ID | | | |
| | NO: 1230) | | NO: 1232) | | | |
| I23RB332 | GYTFTSY | IDPSDSYT | ARSYAFD | QSVDYDG | AAS (SEQ | QQSNEDP |
| | W (SEQ ID | (SEQ ID | Y (SEQ ID | DSY (SEQ | ID | WT (SEQ |
| | NO: 1236) | NO: 1237) | NO: 1238) | ID | NO: 1240) | ID |
| | | | | NO: 1239) | | NO: 1241) |

### Example 2: Binding Kinetics and Affinity Measurements

Affinity of the IL-23R mAbs for human and rat IL-23R ECD protein was determined using both purified and unpurified mAbs by Surface Plasmon Resonance (SPR) and Biolayer Interferometry (BLI) methods. BLI was also used to determine how many different epitopes on human IL-23R ECD are bound by the panel of immunization and phage display derived IL-23R mAbs through competition binding experiments. Commercially available IL-23R mAbs were included in the binding studies to IL-23R protein for comparison. The commercial antibodies evaluated were: Anti-IL23R, Rab Mab, clone EPR22838-4, AbCam cat #222104; Anti-IL23R, Mouse IgG2b, clone #218213, R&D cat #MAB14001; Anti-IL,23R, Mouse IgG1, clone #15N6C6, ThermoFisher cat #MA5-24804; Anti-IL,23R, Mouse IgG1, clone #ABM25G4, Abcam cat #ABM25G4; Anti-IL23R, Mouse IgG2b, LSBio cat #LS-C37068; Anti-IL23R, Mouse IgG₂ₐ, LSBio cat #LS-C308468; and Anti-IL,23R, Mouse IgG1, clone #51H9, LSBio cat #LS-C340289.

### Affinity Measurements

Surface Plasmon Resonance (SPR) and Biolayer Interferometry are label-free detection methods used to investigate biomolecular interactions. By monitoring small changes in mass on a sensor surface, these direct real-time binding assays provide qualitative and quantitative data about the interaction between biomolecules; i.e. determination of equilibrium binding constant (affinity, KD) and kinetic rate constants (ka/kd; rate of complex association ka, and rate of complex dissociation kd). This method is useful in studies of protein-protein and protein-nucleic acid interactions, as well as interactions between proteins and small molecules. Here, interactions between IL-23R antibodies and human IL-23R and rat IL-23R extracellular domain (ECD) proteins were investigated.

The human IL-23R ECD sequence (SEQ ID NO:1) is provided above in Table 3. Sequence comparison and alignment of the human and rat IL-23R extracellular domain sequences demonstrates 76.74% sequence identity. The rat IL-23R ECD sequence (F1LX96 amino acids 24-354) is provided below:

(SEQ ID NO: 1267)
GIATINCSGNMWVEPGEIFQMGMNVSVYCQEALKNCRPRNLHFYKNGFK
ERFHITRINRTTARVWYKGFSEPHASMYCTAECPGRFQETLICGKDISS
GYPPDAPSNMTCVIYEYSGNMTCTWNTGKPTYIDTKYTVHVKSLETEEQ
QQYLASNYVNISTDSLQGGRKYLVWVQAVNALGMENSQQLQVHLDDIVI
PSPSIISRAETTNANVPKTIIYWKSKIMTGKVFCEMRYKATTNQTWNVK
EFDTNYTYVQQSEFYLEPNSKYVFQVRCQGTGKRNWQPWSSPFVHQTPQ
TASQVTSKPPHEPQKIEMLTATIFKGHSTSDNSQDIG

Affinity by SPR using ProteOn. Affinity measurements using Surface Plasmon Resonance (SPR) were performed using a ProteOn XPR36 system (BioRad). A biosensor surface was prepared by coupling anti-Mouse IgG Fc (Jackson cat #315-005-046) to the modified alginate polymer layer surface of a GLC chip (BioRad, Cat #176-5011) using the manufacturer instructions for amine-coupling chemistry. Approximately 6000 RU (response units) of mAbs were immobilized. The kinetic experiments were performed at 25° C. in running buffer (DPBS+0.01% P20+100 μg/ml BSA). To perform kinetic experiments, approximately 250 RU of mAbs were captured followed by injections of human IL-23R at concentrations ranging from 1.56 nM to 400 nM (in a 4-fold serial dilution). The association phase was monitored for 3 minutes at 50 μL/min, then followed by 10 minutes of buffer flow (dissociation phase). The chip surface was regenerated with two 18 second pulses of 100 mM H3PO4 (Sigma, Cat #7961) at 100 μL/min. The collected data were processed using ProteOn Manager software. First, the data was corrected for background using inter-spots. Then, double reference subtraction of the data was performed by using the buffer injection for analyte injections. The kinetic analysis of the data was performed using a Langmuir 1:1 binding model. The result for each mAb was reported in the format of Ka (On-rate), Kd (Off-rate) and KD (equilibrium dissociation constant).

Affinity by SPR using Biacore 8K. Affinity measurements using Surface Plasmon Resonance (SPR) were performed using a Biacore 8K system (Cytiva). A biosensor surface was prepared by coupling anti-Mouse IgG Fc (Jackson cat #315-005-046) to the surface of a CM5 chip using the manufacturer instructions for amine-coupling chemistry. Approximately 9000-10000 RU (response units) of mAbs were immobilized. The kinetic experiments were performed at 25° C. in running buffer 1× of (HBS-P+ Buffer 10× (Cytiva #BR100671)+100 μg/ml BSA). To perform kinetic experiments, approximately 150 RU of mAbs were captured followed by injections of human IL-23R or rat IL-23R at concentrations ranging from 0.8 nM to 500 nM (in a 5-fold serial dilution). The association phase was monitored for 3 minutes at 50 μL/min, then followed by 30 minutes of buffer flow (dissociation phase). The chip surface was regenerated with four 20 second pulses of 0.85% H3PO4 (Sigma, Cat #7961) at 100 μL/min. The collected data were processed using Biacore insight Evaluation software (Version 3.0.12.15655). The kinetic analysis of the data was performed using a Langmuir 1:1 binding model. The result for each mAb was reported in the format of Ka (On-rate), Kd (Off-rate) and KD (equilibrium dissociation constant).

Affinity by BLI using Octet Red 384. Affinity measurements using Biolayer Interferometry (BLI) were performed using an Octet Red 384 system (Sartorius). Antibodies were loaded on to Protein A biosensors (Sartorius, Cat #18-5010) to approximately a 1 nm shift. The kinetic experiments were performed at 25° C. in running buffer of (DPBS+0.02% P20⁺1 mg/ml BSA). To perform kinetic experiments, both human and rat IL-23R are associated at concentrations ranging from 1.37 nM to 1000 nM (in a 3-fold serial dilution). The association phase was monitored for 3 minutes with shaking at 1000 rpm, followed dissociation in buffer for 5 minutes, with shaking at 1000 rpm. The collected data was processed using Octet Evaluation software (Version 10). The kinetic analysis of the data was performed using a Langmuir 1:1 binding model. The result for each mAb was reported in the format of Ka (On-rate), Kd (Off-rate) and KD (equilibrium dissociation constant).

Six of the seven commercially available mAbs tested for binding kinetics by SPR or BLI were mouse IgG mAbs that had weak signal above background or no binding at the highest 4 μM concentration of human IL-23R extracellular domain protein (ECD) tested. The commercially available Abcam rabbit monoclonal antibody (RabMab) (catalog #222104) showed slightly stronger binding to human IL-23R ECD compared with the other 6 commercially available mAbs, but had a KD value >100 nM. The mAbs derived from phage panning and human IL-23R immunizations have a range of affinities from <51 μM to >263 nM and all have better human IL-23R ECD binding affinity than the commercially available R&D Systems human IL-23R mAb, MAB14001. MAB14001 showed no binding to human IL-23R ECD in SPR but had measurable binding to human IL-23R expressing cells in flow cytometry experiments. Most of the phage display and immunization derived mAbs also have better human IL-23R ECD binding affinity than the Abcam mAb (catalog #222104). Only some of the human IL-23R mAbs showed binding to rat IL-23R ECD. Affinities determined for the commercially available human IL-23R binding mAbs and those derived from phage panning and human BL-23R immunizations are summarized in Table 10.

Antibodies 123RB5, 23RB101, I23RB6, I23RB8, 23RB9, I23RB10, and 23RB80 show no binding or undetectable or very weak binding in the assays provided in Table 10. Other antibodies that showed no binding or undetectable or very weak binding are I23RB104, I23RB149, I23RB189, I23RB273, I23RB278, 23RB328, I23RB329, I23RB330 and I23RB332. These antibodies are useful in experiments and assays including as negative control antibodies, providing ‘true’ negatives isolated procedurally with the IL23R binders herein. For instance, I23RB5 has been used as a negative comtrol in various experiments and assays, including as provided herein.

TABLE 10
IL-23R mAb affinities for human and rat IL-23R ECD

| | | SPR data using BIACORE 8K | | | | | | | | | | |
|---|---|---|---|---|---|---|---|---|---|---|---|---|
| | | Binding to human IL-23R ECD (analysis 1) | | | | Binding to human IL-23R ECD (analysis 2) | | | | Binding to Rat IL-23R ECD | | |
| Sample type | mAb ID | ka (1/Ms) | kd (1/s) | KD (M) | Comment | ka (1/Ms) | kd (1/s) | KD (M) | Comment | ka (1/Ms) | kd (1/s) | KD (M) |
| Purified Protein | I23RB1 | | | | | 3.99E+05 | 1.11E−03 | 2.80E−09 | | binding not detectable | | |
| | I23RB3 | | | | | 5.36E+04 | 2.55E−04 | 4.76E−09 | | binding not detectable | | |
| | I23RB4 | | | | | 2.30E+05 | 1.35E−04 | 5.88E−10 | | binding not detectable | | |
| | I23RB5 | | | | | not binder | | | | binding not detectable | | |
| | I23RB7 | | | | | 1.99E+05 | 2.46E−04 | 1.23E−09 | | 2.42E+04 | 1.27E−02 | 5.24E−07 |
| | I23RB94 | 7.87E+05 | 6.30E−04 | 8.00E−10 | | 6.56E+05 | 7.53E−04 | 1.15E−09 | | binding not detectable | | |
| | I23RB92 | 2.65E+05 | <2.89E−05 | <1.09E−10 | slow off rate | 2.40E+05 | <2.89E−05 | <1.20E−10 | slow off rate | binding not detectable | | |
| | I23RB76 | 6.31E+05 | 5.62E−05 | 8.91E−11 | | 4.82E+05 | 4.14E−05 | 8.59E−11 | | binding not detectable | | |
| | I23RB65 | 4.84E+05 | <2.89E−05 | <5.97E−11 | slow off rate | 4.00E+05 | <2.89E−05 | <7.23E−11 | slow off rate | binding not detectable | | |
| | I23RB42 | 7.95E+05 | 5.76E−05 | 7.25E−11 | | 5.35E+05 | 6.25E−05 | 1.17E−10 | | 1.26E+05 | 8.77E−02 | 9.51E−07 |
| | I23RB32 | 5.50E+05 | 1.82E−04 | 3.32E−10 | | 5.06E+05 | 1.72E−04 | 3.40E−10 | | binding not detectable | | |
| | I23RB157 | 3.04E+05 | 8.68E−04 | 2.86E−09 | | 2.30E+05 | 9.47E−04 | 4.12E−09 | | 6.91E+04 | 5.48E−03 | 8.11E−08 |
| | I23RB33 | 9.57E+05 | 1.55E−04 | 1.62E−10 | | 6.91E+05 | 1.49E−04 | 2.16E−10 | | binding not detectable | | |
| | I23RB47 | | | | | 4.53E+05 | 5.17E−05 | 1.14E−10 | | binding not detectable | | |
| | I23RB35 | 5.49E+05 | <2.89E−05 | <5.27E−11 | slow off rate | 4.21E+05 | <2.89E−05 | <6.87E−11 | slow off rate | binding not detectable | | |
| | I23RB101 | No Binding | | | | not binder | | | | binding not detectable | | |
| Unpurified Protein | I23RB1 | | | | | 3.76E+05 | 9.82E−04 | 2.62E−09 | | binding not detectable | | |
| in Mammalian | I23RB3 | | | | | 5.83E+04 | 2.33E−04 | 3.99E−09 | | binding not detectable | | |
| Cell Expression | I23RB4 | | | | | 2.54E+05 | 1.21E−04 | 4.77E−10 | | binding not detectable | | |
| Crude Supernatants | I23RB5 | | | | | not a binder | | | | binding not detectable | | |
| | I23RB7 | | | | | 2.00E+05 | 2.47E−04 | 1.23E−09 | | binding not detectable | | |
| | I23RB94 | | | | | 6.85E+05 | 6.86E−04 | 1.00E−09 | | binding not detectable | | |
| | I23RB92 | | | | | 2.33E+05 | <2.89E−05 | <1.24E−10 | slow off rate | binding not detectable | | |
| | I23RB76 | | | | | 4.84E+05 | 3.90E−05 | 8.05E−11 | | binding not detectable | | |
| | I23RB65 | | | | | 4.16E+05 | 4.58E−05 | 1.10E−10 | | binding not detectable | | |
| | I23RB42 | | | | | 5.82E+05 | 6.29E−05 | 1.08E−10 | | binding not detectable | | |
| | I23RB32 | | | | | not a binder | | | | binding not detectable | | |
| | I23RB157 | | | | | 2.38E+05 | 9.46E−04 | 3.97E−09 | | 2.97E+03 | 5.83E−03 | 1.96E−06 |
| | I23RB33 | | | | | 6.72E+05 | 1.46E−04 | 2.18E−10 | | binding not detectable | | |
| | I23RB47 | 5.66E+05 | <2.89E−05 | <5.10E−11 | slow off rate | 4.45E+05 | 5.32E−05 | 1.19E−10 | | binding not detectable | | |
| | I23RB35 | | | | | 3.72E+05 | <2.89E−05 | <7.77E−11 | slow off rate | binding not detectable | | |
| | I23RB318 | 7.84E+04 | 1.67E−03 | 2.13E−08 | | 7.29E+04 | 1.56E−03 | 2.14E−08 | | binding not detectable | | |
| | I23RB313 | 5.80E+03 | <2.89E−05 | <4.98E−09 | slow off rate | 2.23E+04 | 1.08E−04 | 4.85E−09 | | binding not detectable | | |
| | I23RB270 | 5.35E+05 | 1.02E−03 | 1.91E−09 | | 4.09E+05 | 1.01E−03 | 2.46E−09 | | binding not detectable | | |
| | I23RB269 | 4.73E+05 | 5.14E−04 | 1.09E−09 | | 3.85E+05 | 5.42E−04 | 1.41E−09 | | binding not detectable | | |
| | I23RB268 | 2.28E+04 | 2.73E−04 | 1.20E−08 | | 1.63E+04 | 3.08E−04 | 1.89E−08 | | binding not detectable | | |
| | I23RB267 | 1.08E+04 | 1.96E−04 | 1.81E−08 | | 2.35E+04 | 1.78E−04 | 7.56E−09 | | Binding not detectable | | |
| | I23RB102 | | | | | 4.71E+05 | 8.73E−03 | 1.85E−08 | | Binding not detectable | | |
| | I23RB98 | 7.51E+04 | 2.94E−03 | 3.91E−08 | | 1.36E+05 | 3.50E−03 | 2.58E−08 | | Binding not detectable | | |
| | I23RB93 | 4.80E+05 | 1.38E−03 | 2.88E−09 | | 4.23E+05 | 1.23E−03 | 2.90E−09 | | Binding not detectable | | |
| | I23RB87 | | | | | 8.65E+04 | 9.45E−04 | 1.09E−08 | | Binding not detectable | | |
| | I23RB86 | 7.28E+04 | 5.87E−04 | 8.06E−09 | | 7.03E+04 | 5.72E−04 | 8.14E−09 | | Binding not detectable | | |
| | I23RB85 | 2.10E+05 | 2.26E−04 | 1.08E−09 | | 1.24E+05 | 1.78E−04 | 1.43E−09 | | Binding not detectable | | |
| | I23RB83 | 3.25E+05 | 1.19E−04 | 3.66E−10 | | 1.72E+05 | 5.91E−05 | 3.45E−10 | | Binding not detectable | | |
| | I23RB82 | 5.29E+05 | 2.47E−03 | 4.67E−09 | | 3.97E+05 | 1.36E−02 | 3.44E−08 | | Binding not detectable | | |
| | I23RB81 | not a binder | | | | 1.95E+05 | 1.73E−03 | 8.89E−09 | | Binding not detectable | | |
| | | (possibly | | | | | | | | | | |
| | | switched with | | | | | | | | | | |
| | | I23RB80**) | | | | | | | | | | |
| | I23RB80 | 7.74E+05 | 1.04E−02 | 1.34E−08 | ** | No Binding | | | | Binding not detectable | | |
| | I23RB78 | | | | | 1.79E+05 | 7.74E−04 | 4.33E−09 | | Binding not detectable | | |
| | I23RB77 | 1.39E+06 | 2.14E−03 | 1.54E−09 | | 5.56E+05 | 1.00E−02 | 1.80E−08 | | Binding not detectable | | |
| | I23RB74 | not a binder | | | | 9.54E+05 | 2.22E−03 | 2.33E−09 | | Binding not detectable | | |
| | I23RB73 | 3.22E+05 | 3.78E−03 | 1.17E−08 | | 2.13E+05 | 2.93E−03 | 1.37E−08 | | Binding not detectable | | |
| | I23RB71 | 7.04E+05 | 2.31E−04 | 3.28E−10 | | 3.58E+05 | 4.63E−05 | 1.29E−10 | | Binding not detectable | | |
| | I23RB67 | | | | | 6.77E+05 | 1.72E−02 | 2.54E−08 | | Binding not detectable | | |
| | I23RB61 | 4.59E+05 | 1.01E−02 | 2.21E−08 | | 3.55E+05 | 1.95E−03 | 5.50E−09 | | Binding not detectable | | |
| | I23RB60 | 2.98E+05 | 2.18E−04 | 7.31E−10 | | 3.62E+05 | 1.69E−04 | 4.66E−10 | | Binding not detectable | | |
| | I23RB58 | 3.28E+05 | 5.32E−03 | 1.62E−08 | | 2.33E+05 | 5.23E−03 | 2.25E−08 | | Binding not detectable | | |
| | I23RB57 | 1.02E+05 | 2.99E−03 | 2.92E−08 | | 8.07E+04 | 3.07E−03 | 3.81E−08 | | Binding not detectable | | |
| | I23RB56 | 1.68E+05 | 1.15E−02 | 6.84E−08 | | 3.52E+05 | 8.98E−03 | 2.55E−08 | | 8.97E+04 | 6.42E−02 | 7.15E−07 |
| | I23RB54 | | | | | 5.34E+05 | 9.79E−02 | 1.83E−07 | | Binding not detectable | | |
| | I23RB45 | 1.58E+05 | 3.32E−03 | 2.10E−08 | | 2.03E+05 | 4.56E−03 | 2.25E−08 | | 2.01E+03 | 3.43E−03 | 1.71E−07 |
| | I23RB39 | 2.31E+05 | 7.39E−03 | 3.20E−08 | | 4.82E+05 | 7.16E−03 | 1.49E−08 | | Binding not detectable | | |
| | I23RB36 | 8.64E+04 | 1.18E−03 | 1.37E−08 | | 5.92E+05 | 1.29E−03 | 2.18E−09 | | Binding not detectable | | |
| | I23RB31 | 6.55E+04 | 1.23E−03 | 1.89E−08 | | 9.13E+04 | 7.81E−04 | 8.55E−09 | | Binding not detectable | | |
| | I23RB30 | 2.20E+05 | 1.01E−02 | 4.62E−08 | | 2.12E+05 | 7.63E−03 | 3.59E−08 | | 2.98E+04 | 9.09E−03 | 3.06E−07 |
| | I23RB27 | 3.25E+05 | 3.72E−03 | 1.14E−08 | | 2.23E+05 | 4.16E−03 | 1.87E−08 | | Binding not detectable | | |
| | I23RB188 | 9.82E+04 | 1.30E−03 | 1.33E−08 | | 7.98E+04 | 1.51E−03 | 1.89E−08 | | Binding not detectable | | |
| | I23RB182 | 3.89E+04 | 1.94E−03 | 4.98E−08 | | 9.02E+04 | 3.94E−03 | 4.37E−08 | | Binding not detectable | | |
| | I23RB173 | 2.96E+05 | 7.59E−03 | 2.57E−08 | | 2.85E+05 | 1.03E−02 | 3.61E−08 | | Binding not detectable | | |
| | I23RB169 | 1.28E+05 | 1.16E−02 | 9.13E−08 | | 1.23E+05 | 4.27E−03 | 3.48E−08 | | Binding not detectable | | |
| | I23RB168 | 4.24E+05 | 1.18E−04 | 2.79E−10 | | 3.68E+05 | 1.55E−04 | 4.22E−10 | | Binding not detectable | | |
| | I23RB166 | 4.47E+05 | 1.93E−02 | 4.31E−08 | | 2.14E+05 | 9.42E−03 | 4.41E−08 | | Binding not detectable | | |
| | I23RB165 | 4.46E+05 | 8.06E−03 | 1.81E−08 | | 3.24E+05 | 9.41E−03 | 2.91E−08 | | Binding not detectable | | |
| | I23RB162 | 5.31E+05 | 2.37E−02 | 4.46E−08 | | 3.47E+05 | 2.16E−02 | 6.22E−08 | | Binding not detectable | | |
| | I23RB160 | 5.67E+05 | 9.55E−03 | 1.69E−08 | | 1.02E+06 | 1.42E−02 | 1.38E−08 | | Binding not detectable | | |
| | I23RB156 | 5.42E+05 | 2.18E−04 | 4.03E−10 | | 4.41E+05 | 2.59E−04 | 5.88E−10 | | Binding not detectable | | |
| | I23RB155 | 3.69E+05 | <2.89E−05 | <7.84E−11 | slow off rate | 2.39E+05 | <2.89E−05 | <1.21E−10 | slow off rate | Binding not detectable | | |
| | I23RB153 | 1.38E+05 | 2.26E−04 | 1.64E−09 | | 1.37E+05 | 3.18E−04 | 2.33E−09 | | 5.95E+04 | 5.48E−03 | 9.21E−08 |
| | I23RB150 | 3.41E+05 | 3.29E−02 | 9.65E−08 | | 3.34E+05 | 3.76E−02 | 1.12E−07 | | Binding not detectable | | |
| | I23RB148 | 5.25E+05 | 4.59E−04 | 8.74E−10 | | 4.98E+05 | 3.82E−04 | 7.66E−10 | | Binding not detectable | | |
| | I23RB144 | 8.90E+04 | 2.72E−03 | 3.06E−08 | | 5.20E+04 | 2.77E−03 | 5.33E−08 | | Binding not detectable | | |
| | I23RB260 | 1.84E+04 | 8.72E−05 | 4.75E−09 | | | | | | | | |
| | I23RB91 | 2.14E+04 | 1.10E−04 | 5.15E−09 | | | | | | | | |
| | I23RB177 | 1.88E+04 | 1.07E−04 | 5.70E−09 | | | | | | | | |
| | I23RB319 | 8.85E+03 | 8.01E−05 | 9.05E−09 | | | | | | | | |
| | I23RB259 | 5.59E+04 | 7.52E−04 | 1.34E−08 | | | | | | | | |
| | I23RB264 | 4.74E+04 | 8.47E−04 | 1.79E−08 | | | | | | | | |
| | I23RB321 | 2.09E+04 | 3.84E−04 | 1.84E−08 | | | | | | | | |
| | I23RB261 | 1.79E+04 | 4.02E−04 | 2.25E−08 | | | | | | | | |
| | I23RB317 | 2.36E+04 | 5.96E−04 | 2.52E−08 | | | | | | | | |
| | I23RB161 | 3.24E+05 | 2.74E−02 | 8.45E−08 | | | | | | | | |
| | I23RB180 | 2.01E+05 | 3.02E−02 | 1.50E−07 | | | | | | | | |
| | I23RB69 | 1.88E+05 | 4.93E−02 | 2.63E−07 | | | | | | | | |

| | | SPR data using ProteOn | | | BLI data using Octet | | | | | | |
|---|---|---|---|---|---|---|---|---|---|---|---|
| | | Binding to human IL-23R ECD | | | Binding to human IL-23R ECD | | | | Binding to Rat IL-23R ECD | | |
| Sample type | mAb ID | ka (1/Ms) | kd (1/s) | KD (M) | ka (1/Ms) | kd (1/s) | KD (M) | Comment | ka (1/Ms) | kd (1/s) | KD (M) |
| Purified | I23RB1 | 3.45E+06 | 2.29E−03 | 6.67E−10 | | | | | | | |
| Protein | I23RB2 | Weak binding | | >4.00E−6 | | | | | | | |
| | I23RB3 | 1.74E+05 | 3.35E−04 | 1.92E−09 | | | | | | | |
| | I23RB4 | 1.56E+06 | 2.35E−04 | 1.51E−10 | | | | | | | |
| | I23RB5 | Binding not detectable | | | | | | | | | |
| | I23RB6 | Binding not detectable | | | | | | | | | |
| | I23RB7 | 5.81E+05 | 2.26E−04 | 3.90E−10 | | | | | | | |
| | I23RB8 | Binding not detectable | | | | | | | | | |
| | I23RB9 | Binding not detectable | | | | | | | | | |
| | I23RB10 | Binding not detectable | | | | | | | | | |
| | I23RB157 | | | | 8.56E+04 | 6.60E−05 | 7.71E−10 | estimate | 7.34E+04 | 5.81E−03 | 7.91E−08 |
| Commercial | Anti-IL- | | | | Weak Binding | | | >1.00E−7 | 3.42E+04 | 6.78E−04 | 3.13E−08 |
| Antibodies | 23R, | | | | | | | | | | |
| | Rab | | | | | | | | | | |
| | Mab, | | | | | | | | | | |
| | clone | | | | | | | | | | |
| | EPR22 | | | | | | | | | | |
| | 838-4, | | | | | | | | | | |
| | AbCam | | | | | | | | | | |
| | cat | | | | | | | | | | |
| | #222104 | | | | | | | | | | |
| | Anti-IL- | Binding not detectable | | | | | | | | | |
| | 23R, | | | | | | | | | | |
| | Mouse | | | | | | | | | | |
| | IgG2b, | | | | | | | | | | |
| | clone | | | | | | | | | | |
| | #218213, | | | | | | | | | | |
| | R&D | | | | | | | | | | |
| | cat | | | | | | | | | | |
| | #MAB14001 | | | | | | | | | | |
| | Anti-IL- | Weak binding | | >4.00E−6 | | | | | | | |
| | 23R, | | | | | | | | | | |
| | Mouse | | | | | | | | | | |
| | IgG1, | | | | | | | | | | |
| | clone | | | | | | | | | | |
| | #15N6C6, | | | | | | | | | | |
| | Thermo | | | | | | | | | | |
| | Fisher | | | | | | | | | | |
| | cat | | | | | | | | | | |
| | #MA5- | | | | | | | | | | |
| | 24804 | | | | | | | | | | |
| | Anti-IL- | Weak binding | | >4.00E−6 | | | | | | | |
| | 23R, | | | | | | | | | | |
| | Mouse | | | | | | | | | | |
| | IgG1, | | | | | | | | | | |
| | clone | | | | | | | | | | |
| | #ABM25G4, | | | | | | | | | | |
| | Abcam | | | | | | | | | | |
| | cat | | | | | | | | | | |
| | #ABM25G4 | | | | | | | | | | |
| | Anti-IL- | Binding not detectable | | | | | | | | | |
| | 23R, | | | | | | | | | | |
| | Mouse | | | | | | | | | | |
| | IgG2b, | | | | | | | | | | |
| | LSBio | | | | | | | | | | |
| | cat #LS- | | | | | | | | | | |
| | C37068 | | | | | | | | | | |
| | Anti-IL- | Binding not detectable | | | | | | | | | |
| | 23R, | | | | | | | | | | |
| | Mouse | | | | | | | | | | |
| | IgG2a, | | | | | | | | | | |
| | LSBio | | | | | | | | | | |
| | cat #LS- | | | | | | | | | | |
| | C308468 | | | | | | | | | | |
| | Anti-IL- | Weak binding | | >4.00E−6 | | | | | | | |
| | 23R, | | | | | | | | | | |
| | Mouse | | | | | | | | | | |
| | IgG1, | | | | | | | | | | |
| | clone | | | | | | | | | | |
| | #5H9, | | | | | | | | | | |
| | LSBio | | | | | | | | | | |
| | cat #LS- | | | | | | | | | | |
| | C340289 | | | | | | | | | | |

Note:
samples were not analyzed
If koff is slower than the dissociation limit imposed by the data collection time, koff reported as < the koff limit (Koff is <2.89E−05)
Binding not detectable = No measureable binding signal up to the highest concentration of antigen tested
Weak binding = weak binding signal above background at the highest concentration of antigen tested

### IL-23R mAb Epitope Binning

Recombinant mAbs derived from human IL-23R phage panning and immunizations were paired together and evaluated for competitive binding to the human IL-23R ECD using Biolayer Interferometry (BLI) performed on the Sartorius Octet Red 384 instrument to group them into epitope bins, where mAbs that competed were grouped within the same bin or overlapping bins. If any mAbs did not demonstrate competition when paired with the other mAbs they were classified in their own unique bin. Sufficient binding affinity and/or concentration were required to interpret competitive binding results, and mAbs with affinities and/or concentrations that were too low were not included in an epitope bin with the bin being deemed inconclusive. 4 purified antibodies from the phage panning and 11 purified antibodies from the immunization campaign that exhibited the strongest binding to IL-23R protein, were paired against themselves in a full matrix. The remaining mAbs derived from immunizations or phage display were evaluated as unpurified mAb proteins in supernatants and were only binned against the purified mAb from each bin with the highest affinity.

Epitope Binning by BLI using Octet Red 384. Epitope Binning using Biolayer Interferometry (BLI) was performed using an Octet Red 384 system (Sartorius). To perform epitope binning, experiments were performed at 25° C. in running buffer of (DPBS+0.02% P20⁺1 mg/ml BSA). Biotinylated human IL-23R was loaded on to Streptavidin biosensors (Sartorius, Cat #18-5019) to approximately a 1 nm shift. Antibodies were diluted to 333 nM where concentration allowed. Antibody 1 was associated for 2 minutes. Excess was washed off with a 20 seconds dip into assay buffer. Antibody 2 was associated for 2 minutes. The collected data was processed using epitope binning module with the Octet Evaluation software (Version 10).

Thirty-seven of the anti-IL-23R mAbs derived from phage panning and immunizations were evaluated in epitope binning experiments resulting in at least nine unique bins covering 32 out of the 37 mAbs tested. Three of the remaining five mAbs fell within a unique bin and these mAbs have not been paired with each other, so it is not known if they share the same epitope bin or if there are additional bins within this set. The epitope bins and the mAbs included in each are summarized in Table 11. The diverse set of IL-23R binding sites reflected in these 9(+) epitope bins indicates that, in contrast to the single binding site of MAB14001, the panel of IL-23R mAbs offers a range IL-23R mAb:IL-23R interactions, which may improve receptor binding/detection, either through multiplexing (using a cocktail of multiple IL-23R mAbs functioning in a non-competitive fashion to boost signal) or by targeting a particular epitope that is more accessible to mAb binding.

TABLE 11
Epitope binning of human IL-23R binding mAbs on human IL-23R

| Bin | mAb ID |
|---|---|
| 1 | I23RB42* |
| | I23RB85 |
| | I23RB157* |
| 2 | I23RB32* |
| | I23RB33* |
| | I23RB39 |
| | I23RB60 |
| | I23RB65* |
| | I23RB92* |
| | I23RB94* |
| | I23RB156 |
| | I23RB160 |
| | I23RB162 |
| 3 | I23RB1* |
| | I23RB3* |
| | I23RB4* |
| | I23RB76* |
| | I23RB269 |
| | I23RB270 |
| 4 | I23RB35* |
| | I23RB93 |
| | I23RB155 |
| 5 | I23RB7* |
| 6 (2 & 5) | I23RB47* |
| | I23RB83 |
| | I23RB148 |
| | I23RB168 |
| 7 (2) | I23RB153 |
| 8 (3 & 4) | I23RB36 |
| | I23RB78 |
| 9 (3, 5 & 6) | I23RB45 |
| | I23RB318 |
| Unique | I23RB74 |
| | I23RB77 |
| | I23RB102 |
| Indeterminant | I23RB71 |
| | I23RB313 |
| | AbCam |
| | Ab222104 |

As shown in Table 11, anti-IL-23R mAbs (unpurified in supernatant and purified*) have been grouped into 9 bins using BLI (Octet RED384). mAbs in Bins 6, 7, 8 & 9 are defined as showing competition with at least one mAb in a previously identified Bin(s) shown in parentheses. I23RB74, I23RB77 and I23RB102 are listed as unique based on not competing with the purified antibodies they were tested against. These antibodies have not been tested against each other so they may or may not be in separate bins. mAbs listed as indeterminant are samples that may either be too low in concentration or too weak in affinity for a conclusive result.

### Binding to Human IL-23R Transfected Cell Lines

Recombinant human IL-23R binding mAbs derived from phage panning and immunizations were tested for binding to cell surface human IL-23R expressed on stably transfected iLite and/or HEK cell lines using flow cytometry. The mAbs were tested as either purified proteins or unpurified proteins in crude mammalian cell expression supernatants using either a single or multiple mAb concentrations. Whether the IL-23R mAbs can bind primary human cells expressing IL-23R was also determined by flow cytometry but with purified mAbs only. Commercially available IL-23R mAbs were included in the binding studies to IL-23R expressing cells for comparison.

Human IL-23R iLiTe cell single concentration mAb Binding. Anti-IL-23R mouse IgG₂ₐ mAbs derived from phage panning were tested for binding to human IL-23R expressing iLiTe cells, where a PE conjugated anti mouse secondary antibody detected mAbs bound to the receptor on the cells. Human IL-23R expressing iLite® IL-23 Assay Ready Cells (Eurodiagnostica, Catalog #BM4023) were thawed from cryopreservation, placed in 25 mL RPMI-1640 plus GlutaMAX-1 (ThermoFisher Scientific, Catalog #72400-047) containing 10% FBS (Gibco Life-Technologies, Catalog #16140-063) and 1% Penicillin-Streptomycin (Gibco Life-Technologies, Catalog #15140-122), and incubated overnight at 37° C., 5% CO₂. The next day 100,000 cells were added to each well of a 96 well V bottom plate (Costar, Catalog #3357), centrifuged at 450× gravity for 2 minutes, and then supernatants were decanted. Cells were then resuspended in 100 μL/well of 10% human serum (Valley Biomedical, Catalog #HP1022) in 1×DPBS (Gibco Life-Technologies, Catalog #14190-136) and incubated for 30 minutes on ice followed by centrifugation and decanting of the supernatants. Antibodies diluted to 3 μg/mL in Stain Buffer (BSA) (BD Pharmagen, Catalog #554657) were added at 100 μL/well and incubated on ice for 50 minutes, then plates were centrifuged and supernatants were decanted. Goat anti-mouse PE conjugated secondary antibody (Jackson ImmunoResearch Catalog #115-116-146) diluted 1:200 in Stain Buffer was added at 100 μL/well and incubated on ice for 30 minutes followed by centrifugation and decanting of the supernatants. Cells were washed by adding 100 μL/well of Stain Buffer followed by centrifugation and decanting of the supernatants. Cells were resuspended in 100 μL/well of Stain Buffer and analyzed on the Becton Dickenson LSR II FACS instrument with HTS module. Data was analyzed using FlowJo software (Treestar) to obtain geometric mean fluorescence intensities.

Human IL-23R iLiTe cell concentration response mAb binding. Anti-IL-23R mouse IgG₂ₐ mAbs derived from phage panning were tested for concentration dependent binding to human IL-23R expressing iLiTe cells relative to a mouse IgG₂ₐ isotype negative control mAb and the commercially available MAB14001 positive control mAb from R&D Systems, where a PE conjugated anti mouse secondary antibody detected mAbs bound to the receptor on the cells. Human IL-23R expressing iLite® IL-23 Assay Ready Cells (Eurodiagnostica, Catalog #BM4023) were thawed from cryopreservation, placed in 25 mL RPMI-1640 plus GlutaMAX-1 (ThermoFisher Scientific, Catalog #72400-047) containing 10% FBS (Gibco Life-Technologies, Catalog #16140-063) and 1% Penicillin-Streptomycin (Gibco Life-Technologies, Catalog #15140-122), and incubated overnight at 37° C., 5% CO₂. The next day 100,000 cells were added to each well of a 96 well V bottom plate (Costar, Catalog #3357), centrifuged at 450× gravity for 2 minutes, and then supernatants were decanted. Antibodies were diluted in Stain Buffer (BSA) (BD Pharmagen, Catalog #554657) and added at 50 μL/well, and mixed. Plates were incubated on ice for 50 minutes, then plates were centrifuged and supernatants were decanted. Goat anti-mouse PE conjugated secondary antibody (Jackson ImmunoResearch Catalog #115-116-146) diluted 1:200 in Stain Buffer was added at 100 μL/well and incubated on ice for 30 minutes followed by centrifugation and decanting of the supernatants. Cells were washed by adding 200 μL/well of Stain Buffer followed by centrifugation and decanting of the supernatants. Cells were resuspended in 100 μL/well of Stain Buffer, transferred in quadruplicate to a 384 well plate (Greiner, Catalog #781281) and analyzed on the Becton Dickenson LSR II FACS instrument with HTS module. Data was analyzed using FlowJo software (Treestar) to obtain geometric mean fluorescence intensities, and geometric mean fluorescence intensities were plotted over log mAb concentrations fitted with a sigmoidal dose response curve with variable slope using GraphPad Prism.

Five out of an initial 10 phage derived anti-IL-23R mAbs had measurable binding signals on human IL-23R iLite cells in a single concentration flow cytometry experiment using purified mAb proteins, which include I23RB1, I23RB2, I23RB3, I23RB4, I23RB7 (Table 12, Expt A & B). Subsequent concentration response binding experiments showed all five of these mAbs bound to human IL-23R iLite cells better than the R&D Systems MAB14001 mAb, with binding ranked in the following order I23RB1=I23RB4>I23RB7>I23RB3>I23RB2>MAB14001 (Table 12, Expt C). The lack of binding by I23RB5 repeated, giving the same signal as the mouse IgG₂ₐ isotype control. [00519]83 additional human IL-23R binding mAbs from phage panning and immunizations were also tested for binding to human IL-23R expressing HEK cells as either purified proteins or unpurified proteins in crude expression supernatants from transiently transfected CHO cells.

HEK-IL-23R cell concentration response mAb binding (8-point, 96-well assay). The immunization- and phage-derived anti-IL-23R mouse IgG₂ₐ mAbs were tested for concentration dependent binding to human IL-23R expressing HEK-IL-23R cells relative to a mouse IgG₂ₐ negative control mAb that does not bind to IL-23R on cells, I23RB5, where a 647 conjugated anti mouse secondary antibody detected antibody bound to the receptor on the cells. HEK-IL-23R and HEK-parental (pre-labeled with CellTrackerBlue, ThermoFisher, Cat #C2110) cells were washed with dPBS (Gibco, Cat #14190-136), detached with Accutase (StemCell Technologies, Cat #7920), resuspended in Assay medium (RPMI-1640 (ThermoFisher, Cat #11875135) with 10% fetal bovine serum (Gibco, Cat #A31604-01)), pelleted by centrifugation (3 min, 600 g), and resuspended at 4800 cells/μL. HEK-IL-23R and HEK-parental cells (12.5 μL of each, 120000 cells total) were plated in 2× replicate on 96-well V-bottom plates (Corning, Cat #3357) into 37° C. warmed Assay medium containing serially diluted IL-23R mAbs to a final volume of 150 μL. IL-23R mAbs were allowed to bind to cells for 30 minutes, at 37° C. Cells were next washed twice in ice-cold stain buffer (BD Biosciences, Cat #554657), then exposed to the anti-mouse-647 detection antibody (Jackson ImmunoResearch, Cat #115-606-062) diluted 1:350 in ice-cold stain buffer for 30 minutes on ice. Cells were next washed twice in ice-cold stain buffer, then resuspended in a final volume of 30 μL with Sytox Green viability dye (ThermoFisher, Cat #S7020). Plates were kept on ice until run for analysis on the iQue. All washes were performed by centrifugation (800 rcf, 3 minutes) followed by flick decantation to remove supernatants. Cell-binding to HEK-IL-23R cells was measured with FlowJo, by gating for single, viable cells and determining the geometric mean fluorescence intensity (MFI) for the 647 detection antibody. Geometric mean fluorescence intensities were plotted over log mAb concentrations fitted with a dose response curve with variable slope using GraphPad Prism. Cell binders were scored as those mAbs with MFI signals [S] at the maximum used concentration that were greater than baseline MFI signal [B]+3*StDev, where [B] is the MFI signal detected with the negative control, I23RB5, at an equivalent concentration.

HEK-IL-23R cell concentration response mAb binding (11-point, 384-well assay). The immunization- and phage-derived anti-IL-23R mouse IgG₂ₐ mAbs were tested for concentration dependent binding to human IL-23R expressing HEK-IL-23R cells relative to a mouse IgG₂ₐ negative control mAb that does not bind to IL-23R on cells, I23RB5, where a 647 conjugated anti mouse secondary antibody detected antibody bound to the receptor on the cells. HEK-IL-23R and HEK-parental (pre-labeled with CellTrackerBlue, ThermoFisher, Cat #C2110) cells were washed with dPBS (Gibco, Cat #14190-136), detached with Accutase (StemCell Technologies, Cat #7920), resuspended in Assay medium (RPMI-1640 (ThermoFisher, Cat #11875135) with 10% fetal bovine serum (Gibco, Cat #A31604-01), pelleted by centrifugation (3 min, 600 g), and resuspended at 2500 cells/μL. HEK-IL-23R and HEK-parental cells (10 μL of each, 50000 cells total) were plated in 4× replicate on 384-well V-bottom plates (Greiner, Cat #781281) into 37° C. warmed Assay medium containing serially diluted IL-23R mAbs, respectively, to a final volume of 80 μL. IL-23R mAbs were allowed to bind to cells for 30 minutes, at 37° C. Cells were next washed once in ice-cold stain buffer (BD Biosciences, Cat #554657), then exposed to the anti-mouse-647 detection antibody (Jackson ImmunoResearch, Cat #115-606-062) diluted 1:350 in ice-cold stain buffer for 30 minutes on ice. Cells were next washed twice in ice-cold stain buffer, then resuspended in a final volume of 10 μL with Sytox Green viability dye (ThermoFisher, Cat #S7020). Plates were kept on ice until run for analysis on the iQue. All washes were performed by centrifugation (800 rcf, 3 minutes) followed by automated aspiration to remove supernatants with plate washers. Cell-binding to HEK-IL-23R cells was measured with FlowJo, by gating for single, viable cells and determining the geometric mean fluorescence intensity (MFI) for the 647 detection antibody. Geometric mean fluorescence intensities were plotted over log mAb concentrations fitted with a dose response curve with variable slope using GraphPad Prism. Cell binders were scored as those mAbs with MFI signals [S] at the maximum used concentration that were greater than baseline MFI signal [B]+3*StDev, where [B] is the MFI signal detected with the negative control, I23RB5, at an equivalent concentration.

11 of the 83 additional human IL-23R binding mAbs did not bind cells at thresholds above background (MFI >background * 3× Stdev). Of the 72 IL-23R mAbs that did exhibit cell binding to IL-23R above background, EC50 values ranged from 0.1 nM to >50 nM (Table 12, Expt D & E). Of these cell binders, 34 mAbs presented with MFI (@6.4 nM) values (Table 12, Expt E) that exceeded I23RB3, which together with I23RB2, both showed superior binding compared to MAB14001 [Table 12, Expts A-C(higher MFI, lower EC50)], indicating that these 34 mAbs are also superior to MAB14001 for the detection of IL-23R on cells. These 34 mAbs include I23RB1, I23RB102, I23RB148, I23RB155, I23RB156, I23RB157, I23RB160, I23RB162, I23RB166, I23RB27, I23RB270, I23RB30, I23RB318, I23RB32, I23RB33, I23RB36, I23RB39, I23RB4, I23RB42, I23RB45, I23RB47, I23RB58, I23RB65, I23RB7, I23RB73, I23RB74, I23RB76, I23RB77, I23RB81, I23RB82, I23RB86, I23RB92, I23RB93, and I23RB94. Human IL-23R binding mAbs I23RB71, 123RB165, and 123RB173 had EC50 values ≤9.7 nM (Table 12, Expt E), placing them in the >72 percentile (rank of mAbs Experiment E EC50), and demonstrating they are also superior to MAB14001 for the detection of IL-23R on cells.

The results of IL-23R binding mAbs affinities to human IL-23R expressed on stably transfected iLite and HEK cell lines are shown in Table 12.

TABLE 12
Binding of IL-23R mAbs to human IL-23R expressing cell lines

| | | | human IL-23R iLite cell binding | | | | |
|---|---|---|---|---|---|---|---|
| | | | Expt A | Expt B | Expt C (12-point) | | |
| | | | (1-point) | (1-point) | | Gate 1 + 2 | Gate 2 |
| | | | MFI @ | MFI @ | S > | EC50 | EC50 |
| | | mAb ID | 20 nM | 0.7 nM | B*3xStD | (nM) | (nM) |
| Commercial | | MAB14001 | NT | 280 | Y | ~2.96 | ~12.75 |
| Phage | Purified | I23RB1 | 5086 | 2994 | Y | 0.26 | 0.02 |
| derived | Protein | I23RB2 | 4107 | 670 | Y | 1.09 | 1.11 |
| mAbs | | I23RB3 | 4323 | 1461 | Y | 0.66 | 0.37 |
| | | I23RB4 | 4393 | 2990 | Y | 0.04 | 0.03 |
| | | I23RB5 | 105 | 94 | N | | |
| | | I23RB6 | 139 | NT | NT | NT | NT |
| | | I23RB7 | 4315 | 2231 | Y | 0.07 | 0.10 |
| | | I23RB8 | 105 | NT | NT | NT | NT |
| | | I23RB9 | 86 | NT | NT | NT | NT |
| | | I23RB10 | 359 | NT | NT | NT | NT |

| | | | human IL-23R HEK cell binding | | | | Percentile rank of mAbs | | |
|---|---|---|---|---|---|---|---|---|---|
| | | | Expt D (8-point, | | Expt E (11-point, | | (Top performer = 100%) | | |
| | | | 96well) | | 384well) | | | Expt E | |
| | | | S > | EC50 | MFI | EC50 | Expt D | (MFI @ | Expt E |
| | | mAb ID | B*3xStD | (nM) | (6.4 nM) | (nM) | (EC50) | 6.4 nM) | (EC50) |
| Commercial | | MAB14001 | NT | NT | NT | NT | NT | NT | NT |
| Phage | Purified | I23RB1 | NT | NT | 286726 | NT | NT | 92 | NT |
| derived | Protein | I23RB2 | NT | NT | NT | NT | NT | NT | NT |
| mAbs | | I23RB3 | NT | NT | 148614 | NT | NT | 61 | NT |
| | | I23RB4 | NT | NT | 224800 | NT | NT | 84 | NT |
| | | I23RB5 | NT | NT | 3915 | NT | NT | <31 | NT |
| | | I23RB6 | NT | NT | NT | NT | NT | NT | NT |
| | | I23RB7 | NT | NT | 171633 | NT | NT | 68 | NT |
| | | I23RB8 | NT | NT | NT | NT | NT | NT | NT |
| | | I23RB9 | NT | NT | NT | NT | NT | NT | NT |
| | | I23RB10 | NT | NT | NT | NT | NT | NT | NT |
| | Unpurified | I23RB321 | Y | 18.6 | NT | NT | 32 | <31 | <32 |
| | Protein in | I23RB319 | Y | 8.7 | NT | NT | 38 | <31 | <32 |
| | Mammalian | I23RB318 | Y | 0.5 | 196302 | 7.1 | 71 | 80 | 83 |
| | Cell | I23RB317 | Y | 31.0 | NT | NT | 25 | <31 | <32 |
| | Expression | I23RB313 | Y | >50.0 | NT | NT | 16-22 | <31 | <32 |
| | Crude | I23RB278 | N | | NT | NT | <14 | <31 | <32 |
| | Supernatants | I23RB277 | Y | >50.0 | NT | NT | 16-22 | <31 | <32 |
| | | I23RB273 | N | | NT | NT | <14 | <31 | <32 |
| | | I23RB270 | Y | 0.5 | 243373 | 7.2 | 74 | 86 | 80 |
| | | I23RB269 | Y | 0.6 | 96789 | 14.7 | 66 | 49 | 60 |
| | | I23RB268 | Y | 20.6 | 51882 | 38.2 | 31 | 36 | 46 |
| | | I23RB267 | Y | 9.4 | NT | NT | 35 | <31 | <32 |
| | | I23RB264 | Y | >50.0 | NT | NT | 16-22 | <31 | <32 |
| | | I23RB261 | Y | 9.2 | NT | NT | 36 | <31 | <32 |
| | | I23RB260 | Y | >50.0 | NT | NT | 16-22 | <31 | <32 |
| | | I23RB259 | Y | 29.7 | 30882 | >50.0 | 27 | 33 | 33-45 |
| | | I23RB332 | N | | NT | NT | <14 | <31 | <32 |
| | | I23RB330 | N | | NT | NT | <14 | <31 | <32 |
| | | I23RB329 | N | | NT | NT | <14 | <31 | <32 |
| | | I23RB328 | N | | NT | NT | <14 | <31 | <32 |
| Immu | Purified | I23RB94 | Y | 0.1 | 363988 | 2.8 | 99 | 99 | 99 |
| | | I23RB92 | Y | 0.2 | 375866 | 4.0 | 97 | 100 | 94 |
| | | I23RB76 | Y | 0.4 | 321510 | 11.9 | 79 | 95 | 63 |
| | | I23RB65 | Y | 0.3 | 274916 | 4.0 | 86 | 90 | 93 |
| | | I23RB42 | Y | 0.2 | 352620 | 3.1 | 92 | 97 | 98 |
| | | I23RB32 | Y | 0.4 | 292423 | 5.1 | 77 | 93 | 88 |
| | | I23RB157 | NT | NT | 180188 | 11.0 | NT | 71 | 68 |
| | | I23RB33 | Y | 0.2 | 305792 | 5.6 | 95 | 94 | 85 |
| | | I23RB47 | Y | 0.2 | 363504 | 4.1 | 91 | 98 | 91 |
| | | I23RB35 | Y | 0.2 | NT | NT | 96 | NT | NT |
| | | I23RB101 | N | | NT | NT | <14 | <31 | <32 |
| | Unpurified | I23RB104 | N | | NT | NT | <14 | <31 | <32 |
| | Protein in | I23RB102 | NT | NT | 257715 | 8.6 | NT | 87 | 76 |
| | Mammalian | I23RB98 | Y | 1.9 | 59393 | 28.7 | 47 | 38 | 50 |
| | Cell | I23RB95 | NT | NT | NT | NT | NT | NT | NT |
| | Expression | I23RB93 | Y | 0.5 | 192659 | 8.9 | 73 | 79 | 73 |
| | Crude | I23RB91 | Y | 30.3 | NT | NT | 26 | <31 | <32 |
| | Supernatants | I23RB87 | NT | NT | 86143 | >50.0 | NT | 46 | 33-45 |
| | | I23RB86 | Y | 1.2 | 164436 | 22.8 | 52 | 66 | 52 |
| | | I23RB85 | Y | ~0.6 | 64033 | 13.5 | 68 | 40 | 61 |
| | | I23RB83 | Y | 0.7 | 120645 | 31.5 | 60 | 59 | 49 |
| | | I23RB82 | Y | 0.6 | 180983 | 9.8 | 69 | 74 | 71 |
| | | I23RB81 | Y | 0.9 | 180907 | 7.2 | 55 | 72 | 82 |
| | | I23RB80 | N | | NT | NT | <14 | <31 | <32 |
| | | I23RB79 | NT | NT | NT | NT | NT | NT | NT |
| | | I23RB78 | NT | NT | 84244 | 15.8 | NT | 45 | 57 |
| | | I23RB77 | Y | 0.2 | 183687 | 4.8 | 94 | 75 | 89 |
| | | I23RB74 | Y | 0.3 | 184983 | 13.0 | 84 | 77 | 62 |
| | | I23RB73 | Y | 1.8 | 157826 | 11.6 | 48 | 64 | 67 |
| | | I23RB71 | Y | ~6.7 | 109959 | 3.4 | 39 | 54 | 96 |
| | | I23RB69 | Y | 0.6 | NT | NT | 65 | <31 | <32 |
| | | I23RB67 | NT | NT | 80940 | 18.0 | NT | 44 | 55 |
| | | I23RB66 | NT | NT | NT | NT | NT | NT | NT |
| | | I23RB61 | Y | 0.6 | 117821 | >50.0 | 70 | 57 | 33-45 |
| | | I23RB60 | Y | 0.6 | 93733 | 27.7 | 62 | 48 | 51 |
| | | I23RB58 | Y | 23.1 | 149794 | 11.6 | 30 | 62 | 65 |
| | | I23RB57 | Y | 5.0 | 99600 | >50.0 | 40 | 51 | 33-45 |
| | | I23RB56 | Y | 0.7 | 117690 | >50.0 | 61 | 56 | 33-45 |
| | | I23RB54 | NT | NT | 63092 | 35.2 | NT | 39 | 48 |
| | | I23RB45 | Y | 0.4 | 227725 | 4.5 | 78 | 85 | 90 |
| | | I23RB39 | Y | 0.3 | 277390 | 3.8 | 90 | 91 | 95 |
| | | I23RB36 | Y | 0.4 | 170901 | 7.3 | 81 | 67 | 79 |
| | | I23RB31 | Y | 1.7 | 78646 | >50.0 | 49 | 43 | 33-45 |
| | | I23RB30 | Y | 1.2 | 184569 | 8.3 | 51 | 76 | 77 |
| | | I23RB27 | Y | 3.5 | 152591 | >50.0 | 43 | 63 | 33-45 |
| | | I23RB189 | N | | NT | NT | <14 | <31 | <32 |
| | | I23RB188 | Y | 2.0 | 90088 | >50.0 | 45 | 47 | 33-45 |
| | | I23RB182 | Y | 36.4 | 26205 | >50.0 | 23 | 32 | 33-45 |
| | | I23RB180 | Y | 27.6 | NT | NT | 29 | <31 | <32 |
| | | I23RB177 | Y | >50.0 | NT | NT | 16-22 | <31 | <32 |
| | | I23RB173 | Y | 0.4 | 100271 | 7.4 | 82 | 52 | 78 |
| | | I23RB169 | Y | 0.7 | 131724 | 15.4 | 58 | 60 | 59 |
| | | I23RB168 | Y | 3.4 | 73043 | 22.1 | 44 | 41 | 54 |
| | | I23RB166 | Y | 0.3 | 197533 | 11.6 | 87 | 82 | 66 |
| | | I23RB165 | Y | 0.6 | 116290 | 9.7 | 64 | 55 | 72 |
| | | I23RB162 | Y | 0.1 | 269100 | 2.8 | 100 | 89 | 100 |
| | | I23RB161 | Y | 0.8 | NT | NT | 56 | <31 | <32 |
| | | I23RB160 | Y | 0.4 | 185189 | 5.6 | 75 | 78 | 87 |
| | | I23RB159 | Y | >50.0 | NT | NT | 16-22 | <31 | <32 |
| | | I23RB156 | Y | 0.4 | 174305 | 8.9 | 83 | 69 | 74 |
| | | I23RB155 | Y | 0.9 | 177295 | 10.1 | 53 | 70 | 70 |
| | | I23RB153 | Y | 4.4 | 52771 | >50.0 | 42 | 37 | 33-45 |
| | | I23RB150 | Y | 0.8 | 102513 | 16.2 | 57 | 53 | 56 |
| | | I23RB149 | N | | NT | NT | <14 | <31 | <32 |
| | | I23RB148 | Y | 0.3 | 202907 | 6.0 | 88 | 83 | 84 |
| | | I23RB144 | Y | 13.4 | 46250 | >50.0 | 34 | 34 | 33-45 |

NT—Not tested in this experiment
N—No cell binding observed
S—Geometric mean fluorescence intensity value for I23RB mAb at max concentration
B—Geometric mean fluorescence intensity value for I23RB5 negative control at equivalent concentration
StD—Standard deviation for I23RB5 negative control across replicate wells
MFI—Geometric mean fluorescence intensity of the detection antibody
Black filled cells indicate EC50 could not be calculated due to no observable cell binding
EC50 values that exceed the maximal [mAb] used in the experiment can not accurately be quantified and are listed as “>50”

mAbs ranking is shown in Table 12. For Experiment D, the 77 mAbs tested were ranked in ascending order by EC50. The relative position of all mAbs is indicated by the %, with the top performers (lowest EC50's) showing the highest %'s. A percentage range is included for mAbs that could not be accurately quantified due to poor cell binding or EC50's >50. For Experiment E (MFI), the 61 mAbs tested were ranked in descending order by MFI at 6.4 nM. The relative position of the mAbs is indicated by the %, with the top performers (highest MFI's) showing the highest %'s. 26 mAbs that were not tested because of poor cell binding in Experiment D were included in the rankings and pre-ranked at the bottom (range <31%). For experiment E (EC50), the 56 mAbs tested were ranked in ascending order by EC50 values. The relative position of the mAbs is indicated by the %, with the top performers (lowest EC50's) showing the highest %'s. 26 mAbs that were not tested because of poor cell binding in Experiment D were included in the rankings and pre-ranked at the bottom (range <32%). A percentage range is included for mAbs that could not be accurately quantified due to EC50's >50.

### Example 3: IL-23R mAbs Detect IL-23R on the Surface of Primary Human Immune Cells

Primary cell culture. Cryopreserved peripheral blood mononuclear cells (PBMCs) from healthy donors (AllCells) were thawed and washed twice in ImmunoCult-XF T cell expansion medium (XF-TCEM) (StemCell Technologies cat #10981) supplemented with CTL anti-aggregate wash (Cellular Technology Limited cat #CTL-AA-001). The cells were counted, resuspended at 2-6×10⁵ cells per mL XF-TCEM supplemented with penicillin and streptomycin (Sigma-Aldrich cat #P0781-100 ML) and 100 ng/mL IL-1β(Biolegend cat #579404), and cultured in flasks coated with 10 μg/mL anti-CD3 (clone UCHT1) (BD Pharmingen cat #555329) for 4 days at 37° C. in 5% CO₂. On day 4 of culture, PBMCs were collected, washed twice in RPMI-1640 supplemented with 0.1% BSA (RPMI-BSA), and counted. Cells were stained directly with anti-IL-23R mAbs or incubated in RPMI-BSA in upright tissue culture flasks for >4 hours at 37° C. in 5% CO₂ prior to staining. When functional IL-23R expression (i.e., IL-23 responsiveness) was assessed, cells were incubated in RPMI-BSA in upright tissue culture flasks for >4 hours at 37° C. in 5% CO₂.

Measurement of IL-23-induced STAT3 phosphorylation. To assess IL-23 responsiveness, a total of 6×10⁴ ‘cytokine-starved’ cells in 30 μL RPMI-BSA was transferred into each well of a 384-well plate. The cells were stimulated with a serial titration of IL-23 (internally sourced from Janssen). After a 30-minute incubation at 37° C. in 5% CO₂, the cells were transferred onto ice for 10 minutes and then lysed. Cell lysates were stored at −80° C. After thawing at room temperature, 25 μL of each lysate were transferred to MSD plates treated with ‘Blocker A’ provided with the MSD phospho-STAT panel kit (Meso Scale Discovery cat #K15202D). Following a one-hour incubation with shaking, the plates were washed with Tris wash buffer. A total of 25 μL detection antibodies was added to each well, and the plates were incubated for an additional hour with shaking. The plates were then washed three times. After the final wash, 150 μL read buffer were added to each well before reading the plates on a Meso Sector S 600 plate reader. Phosphorylated STAT3 levels (i.e., raw MSD plate reads) were plotted vs IL-23 concentration in GraphPad Prism. The EC₅₀ value for IL-23-induced STAT3 phosphorylation was calculated in GraphPad Prism using non-linear regression (curve fit)—[inhibitor] vs response (four-parameters)—robust regression.

An initial set of phage display-derived anti-IL-23R mAbs was tested for detection of IL-23R on the surface of primary immune cells by staining PBMCs that had been stimulated for 4 days with anti-CD3 in the presence of IL-1P, conditions that induce IL-23 responsiveness and by extension, IL-23R expression.

Surface IL-23R staining. For flow cytometric detection of IL-23R, up to ˜10⁶ cells were incubated in 50-100 μL Dulbecco's phosphate buffered saline (PBS) supplemented with 0.2% bovine serum albumin (BSA) or FACS staining buffer with BSA (BD Pharmingen cat #554657) supplemented with FcR blocking reagent (Miltenyi Biotec cat #130-059-901) and Ghost dye red 780 (Tonbo Biosciences cat #13-0865-T100). Following a 5-minute incubation on ice, 50 μL FACS staining buffer containing 20-30 μg/mL anti-IL-23R mAb were added to each sample such that the final concentration of antibody was 10 μg/mL. Cells were incubated for 60 minutes on ice and washed with FACS staining buffer. Cell pellets were resuspended in 100 μL FACS staining buffer containing biotinylated anti-mouse IgG₂ₐ secondary antibody (BD Pharmingen cat #550332) and incubated for 30 minutes on ice. After a second wash, cells were incubated in 100 μL FACS staining buffer containing streptavidin (SAV)-PE (BD Pharmingen cat #349023 or Biolegend cat #405203) and fluorescently labeled antibodies against relevant cell surface markers (Miltenyi Biotec and BD Pharmingen). Cells were acquired on a BD FACSCanto or FACSymphony cell analyzer, and data were analyzed using FlowJo software.

To amplify the IL-23R signal, a three-step staining strategy was employed in which bound anti-IL-23R was detected with a biotinylated anti-mouse IgG₂ₐ secondary antibody and streptavidin-PE. To establish a ‘cut-off’ for anti-IL-23R mAb binding, cells were incubated with buffer alone or the negative control mAb, I23RB5, during the first step of the three-step staining protocol. As an additional control, the biotinylated anti-mouse IgG₂ₐ secondary reagent was omitted from the second staining step.

As shown in FIG. 4, the omission of either the primary or second step antibodies as well as three-step staining with I23RB5 resulted in low or no background staining on CD8⁺ and CD8⁻ CD3⁺ CD56⁻ T cells and CD3⁻ CD56⁺ cells, respectively. A moderate level of nonspecific SAV-PE binding was detected on the surface of CD3⁺ CD56⁺ cells (FIG. 4). The phage display-derived anti-IL-23R mAbs I23RB1, I23RB2, I23RB3, I23RB4, and I23RB7 bound CD8⁺ and CD8⁻ CD3⁺ CD56⁻ T cells (FIG. 5, part A) and a small but detectable number of CD3⁻ CD56⁺ cells (FIG. 5, part C). A majority of CD3⁺ CD56⁺ cells was stained with each phage display-derived anti-IL-23R mAb evaluated (FIG. 5, part B).

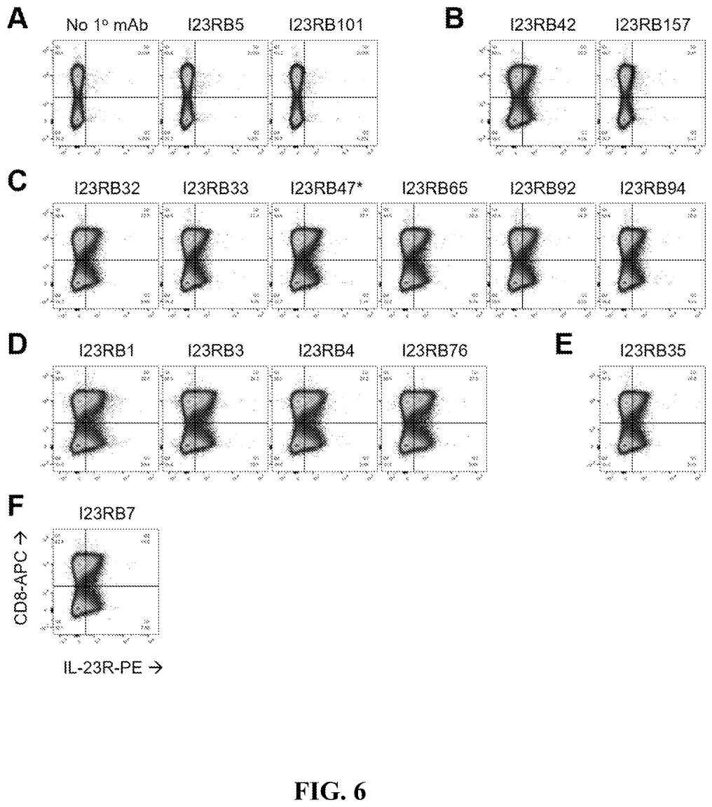

Two additional sets of anti-IL-23R mAbs derived from immunizing mice with the extracellular domain of human IL-23R were tested as described above for flow cytometric detection of IL-23R on the surface of primary human immune cells. Of 26 mouse immunization-derived mAbs tested, 22 bound CD8⁺ and CD8⁻ CD3⁺ CD56⁻ T cells to varying degrees including I23RB42, I23RB32, I23RB33, I23RB47, I23RB65, I23RB92, I23RB94, I23RB76, I23RB35, I23RB36, I23RB45, I23RB77, I23RB78, I23RB83, I23RB85, I23RB148, I23RB153, I23RB155, I23RB156, I23RB168, I23RB269, and 123RB313 (representative data shown in FIG. 6). Of the immunization-derived mAbs, only I23RB76 gave equivalent (or improved) signal-to-noise relative to I23RB1, I23RB3, and I23RB4, the strongest cell binders among the previously identified phage display-derived mAbs. Notably, these four mAbs compete for binding to the IL-23R extracellular domain and thus, belong to the same ‘epitope bin.’

### Example 4: Confirmation of IL-23R mAb Binding Specificity by siRNA-Mediated IL23R Knock-Down

To confirm the specificity of the anti-IL-23R mAbs for IL-23R, PBMCs that had been stimulated for 3 days with anti-CD3 and IL-1P were electroporated with a mixture of siRNA targeting the IL23R transcript. Following electroporation, the cells were placed back into culture on anti-CD3-coated flasks for ˜24 hours, at which time the cells were pelleted for RNA isolation or stained for surface IL-23R and IL-12RP1 expression.

siRNA-mediated IL23R knock-down. PBMCs were cultured on anti-CD3-coated flasks in serum-free medium supplemented with 100 ng/mL IL-1β. On day 3 of culture, ˜10⁷ cells were resuspended in 200 μL electroporation buffer provided with the P3 Primary Cell 4D-Nucleofector X Kit S (Lonza cat #V4XP-3032). A mixture of three siRNA molecules targeting the IL23R gene (Thermo Fisher Scientific cat #AM16708 assay ID 110064, 110065, 110066) or a negative control siRNA (Thermo Fisher Scientific cat #AM4611) was added to the cells such that the final concentration of siRNA was 300 nM (100 nM of each individual IL23R siRNA or 300 nM of the negative control siRNA). Following electroporation, the cells were placed back into anti-CD3-coated flasks and cultured for an additional ˜24 hours. At that time, cells were stained for surface IL-23R and IL-12Rβ1 expression or pelleted for RNA isolation (Qiagen cat #74004). Isolated RNA was reverse transcribed into cDNA (Applied Biosystems cat #4374967) and IL23R and IL12RB1 transcript levels were analyzed by quantitative polymerase chain reaction (qPCR) (Thermo Fisher Scientific cat #4304437) using probes for IL23R (Hs00332759_ml), IL12RB1 (Hs01106578_ml), ACTB (Hs99999903_ml), B2M (Hs00187842_ml), GAPDH (Hs02786624_g1), and HPRT1 (Hs02800695_ml).

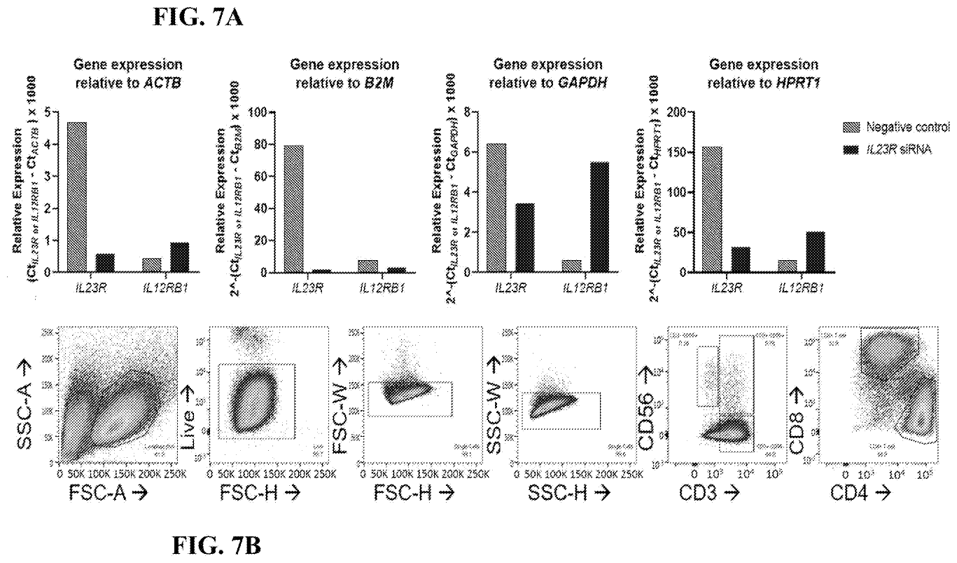

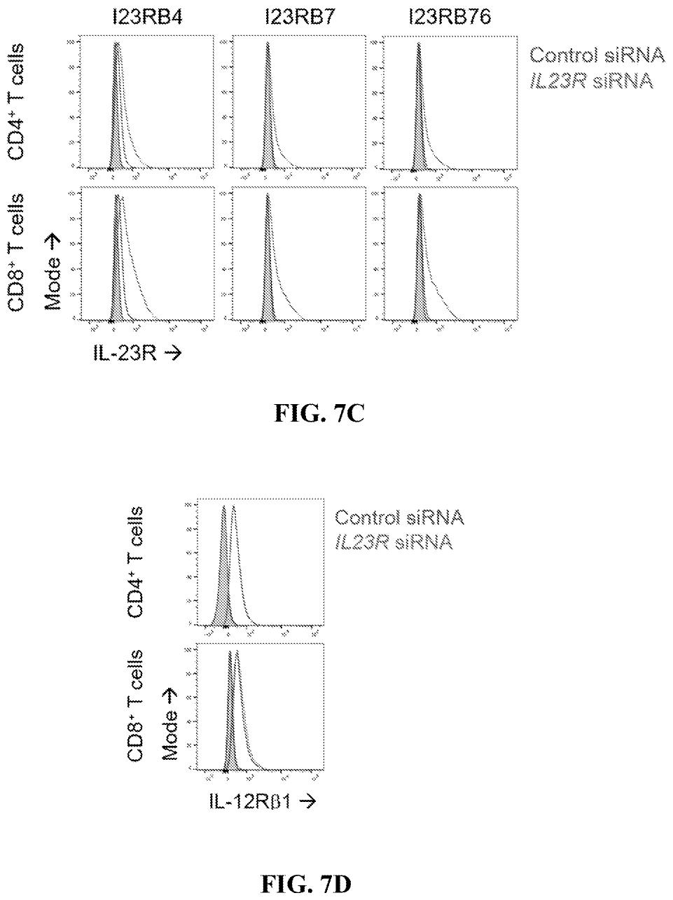

siRNA-mediated IL23R knock-down resulted in a ˜1.9 to 38-fold reduction in IL23R transcript levels compared with cells electroporated with a negative control siRNA (FIG. 7, part A). Transcript levels of IL12RB1 were either reduced or increased by IL23R knock-down depending on the housekeeping gene used for normalization (FIG. 7, part A).

Relative to cells electroporated with negative control siRNA, IL23R knock-down reduced I23RB4, I23RB7, and I23RB76 binding to CD4⁺ and CD8⁺ CD3⁺ CD56⁻ T cells (FIG. 7, parts B and C). Cells electroporated with IL23R or control siRNA expressed equivalent surface levels of IL-12RP1 (FIG. 7, part D). Thus, I23RB4, I23RB7, and I23RB76 specifically detect IL-23R on the surface of primary human CD3⁺ CD56⁻ T cells.

### Example 5: Antibody Cross-Reactive Binding to Rat IL-23R

Rat IL-23R cross-reactive binding was demonstrated in seven anti-human IL-23R antibodies that fall into several epitope bins, particularly falling into distinct epitope bins. Cross reactive binding is important and useful for application of the IL-23R antibodies in rats in vivo, ex vivo or in vitro (flow or other) studies without needing to generate specific anti-rat IL-23R surrogate antibodies with properties that approximately match those of any particular anti-human IL-23R mAb of interest. The generation of such surrogate mAbs can often be very challenging and require as much or even more work than that which was required to generate the initial human IL-23R antibodies. For these reasons, the anti-human IL-23R antibodies with rat IL-23R cross-reactive binding are of particular value.

Table 13 below is modified from Tables 10 and 11 to indicate the antibody epitope binning results and also focus on the human and rat IL-23R binding of antibodies that demonstrated rat IL-23R binding. Antibody binding characteristics as available for antibodies in each of the epitope bins is provided. Antibodies in each of Bins 1, 5, 7, and 9 and also not binned antibodies I23RB30 and I23RB56 demonstrate rat IL-23R binding. Antibodies I23RB30 and I23RB56 have not been epitope binned due to not meeting criteria used to include mAbs in the epitope binning experiments. This view of the binding information shows that two of these mAbs (I23RB7, I23RB153) are the only mAb in their respective epitope bins (#5, #7). Two mAbs (I23RB42, I23RB157) fall within the same bin (Bin 1). Bin 1 contains a third mAb (I23RB85) which did not demonstrate rat IL-23R cross-reactivity in these studies, but which is predicted to bind rat IL-23R. A mAb within Bin 1 would be expected to be rat cross-reactive based on the phenotype of the other Bin 1 mAbs. This lack of rat IL-23R binding by I23RB85 in the studies can be plausibly explained in a couple of possible ways. I23RB85 was assessed for rat IL-23R binding using non-purified protein in expression culture supernatant. In this form, the amount of antibody could be lower than estimated and therefore be at a concentration that does not allow detection of binding. This is observed with I23RB42, which shows rat IL-23R binding when using purified protein but not when using non-purified protein in expression supernatant (Table 10). Also, I23RB85 could have lower affinity for rat IL-23R than for human IL-23R and with that lower affinity, not show detectable binding under the experimental conditions used in the binding studies. 123RB7 is an example of a mAb with lower affinity to rat IL-23R than to human IL-23R. The rat cross-reactive mAb I23RB45 falls into another distinct epitope bin (9). Epitope Bin 9 contains a second mAb, I23RB318, which does not show binding to rat IL-23R. This lack of rat IL-23R binding, which would be unexpected, can be explained in the same was as is explained above for 23RB85.

Thus, overall, the following nine IL-23R antibodies have been shown to bind and/or are predicted to bind to rat IL-23R, based on rat binding studies and epitope binning: I23RB42, I23RB85, I23RB157, I23RB7, I23RB153, 23RB45, I23RB318, I23RB30 and I23RB56.

TABLE 13
Epitope binning and rat IL-23R binding
of human IL-23R binding mAbs

| | | Rat IL-23R ECD | Affinity to hu IL- |
|---|---|---|---|
| Bin | mAb ID | binder | 23R ECD (nm) |
| 1 | I23RB42* | yes | ≤0.1 |
| | I23RB85 | | 1 |
| | I23RB157* | yes | 3-4 |
| 2 | I23RB32* | | |
| | I23RB33* | | |
| | I23RB39 | | |
| | I23RB60 | | |
| | I23RB65* | | |
| | I23RB92* | | |
| | I23RB94* | | |
| | I23RB156 | | |
| | I23RB160 | | |
| | I23RB162 | | |
| 3 | I23RB1* | | |
| | I23RB3* | | |
| | I23RB4* | | |
| | I23RB76* | | |
| | I23RB269 | | |
| | I23RB270 | | |
| 4 | I23RB35* | | |
| | I23RB93 | | |
| | I23RB155 | | |
| 5 | I23RB7* | yes | 1 |
| 6 (2 & 5) | I23RB47* | | |
| | I23RB83 | | |
| | I23RB148 | | |
| | I23RB168 | | |
| 7 (2) | I23RB153 | yes | 2 |
| 8 (3 & 4) | I23RB36 | | |
| | I23RB78 | | |
| 9 (3, 5 & 6) | I23RB45 | yes | 2 |
| | I23RB318 | | 21 |
| Unique | I23RB74 | | |
| | I23RB77 | | |
| | I23RB102 | | |
| Indeterminant | I23RB71 | | |
| | I23RB313 | | |
| Not binned | I23RB30 | yes | 36-46 |
| | I23RB56 | yes | 26-68 |

### Example 6: Antibody CDR and Variable Region Sequences

Tables 4-6 and 7-9 provide Heavy chain (HC), light chain (LC), variable region heavy chain (VH) and light chain (VL) and heavy and light chain CDR sequences for the IL-23R antibodies described and provided herein. Antibody fragments, particularly active or functional fragments or antigen binding fragments, may include or comprise the respective CDR sequences or the VH or VL sequence or the HC or LC sequence of the antibodies disclosed herein, or portions thereof. A review of the sequences identifies antibodies with related, similar or identical sequences. Antibodies are below designated in five groupings, wherein the antibodies have identical LC, VH and light chain CDR1, CDR2 and CDR3 sequences. In each instance, however, the antibodies have distinct or unque HC, VH and heavy chain CDR1, CDR2 and CDR3 sequences.

For instance, some antibodies share light chain CDRs, and also may share LC and/or VL sequence. Notably these antibodies are distinct and unique in heavy chain sequence, including heavy chain CDRs and HC and VH sequences. The heavy chain sequences, including particularly the CDR sequences provided in and for these antibodies, are particularly responsible for binding differences or characteristics of these antibodies. Some bin together in the same epitope bin, while some are in distinct epitope bins or are uncharacterized by the bin analysis conducted. The below Table 13 identifies shared consensus light chain LC amino acid sequences and the corresponding IL-23R antibodies comprising the LC sequence. Table 14 identifies shared consensus VL sequences and the corresponding IL-23R antibodies comprising the VL sequence. Table 14 identifies shared consensus light chain CDR1, CDR2 and CDR3 sequences and the corresponding IL-23R antibodies comprising the CDR sequences.

TABLE 13
Shared Antibody LC Amino Acid Sequences

| | Corresponding Identical | Antibodies Comprising |
|---|---|---|
| LC Sequence and Consensus SEQ ID | Sequences and SEQ IDs | LC sequence |
| EIVLTQSPATLSLSPGERATLSCRASQS | SEQ ID NO: 673 | I23RB1 |
| VSSYLAWYQQKPGQAPRLLIYDASNR | SEQ ID NO: 675 | I23RB2 |
| ATGIPARFSGSGSGTDFTLTISSLEPEDF | SEQ ID NO: 677 | I23RB3 |
| AVYYCQQRSNWPLTFGQGTKVEIKRA | SEQ ID NO: 679 | I23RB4 |
| DAAPTVSIFPPSSEQLTSGGASVVCFLN | SEQ ID NO: 687 | I23RB8 |
| NFYPKDINVKWKIDGSERQNGVLNSW | SEQ ID NO: 689 | I23RB9 |
| TDQDSKDSTYSMSSTLTLTKDEYERHN | SEQ ID NO: 711 | I23RB268 |
| SYTCEATHKTSTSPIVKSFNRNEC | SEQ ID NO: 715 | I23RB270 |
| (SEQ ID NO: 1242) | SEQ ID NO: 757 | I23RB291 |
| DIQMTQSPSSLSASVGDRVTITCRASQS | SEQ ID NO: 681 | I23RB5 |
| ISSYLNWYQQKPGKAPKLLIYAASSLQ | SEQ ID NO: 707 | I23RB266 |
| SGVPSRFSGSGSGTDFTLTISSLQPEDF | SEQ ID NO: 717 | I23RB271 |
| ATYYCQQSYSTPLTFGQGTKVEIKRAD | SEQ ID NO: 721 | I23RB273 |
| AAPTVSIFPPSSEQLTSGGASVVCFLNN | SEQ ID NO: 723 | I23RB274 |
| FYPKDINVKWKIDGSERQNGVLNSWT | SEQ ID NO: 725 | I23RB275 |
| DQDSKDSTYSMSSTLTLTKDEYERHNS | SEQ ID NO: 727 | I23RB276 |
| YTCEATHKTSTSPIVKSFNRNEC | SEQ ID NO: 729 | I23RB277 |
| (SEQ ID NO: 1243) | SEQ ID NO: 733 | I23RB279 |
| | SEQ ID NO: 737 | I23RB281 |
| | SEQ ID NO: 739 | I23RB282 |
| | SEQ ID NO: 743 | I23RB284 |
| | SEQ ID NO: 745 | I23RB286 |
| | SEQ ID NO: 753 | I23RB289 |
| | SEQ ID NO: 755 | I23RB290 |
| | SEQ ID NO: 759 | I23RB292 |
| DIVMTQSPDSLAVSLGERATINCKSSQS | SEQ ID NO: 683 | I23RB6 |
| VLYSSNNKNYLAWYQQKPGQPPKLLI | SEQ ID NO: 691 | I23RB10 |
| YWASTRESGVPDRFSGSGSGTDFTLTIS | | |
| SLQAEDVAVYYCQQYYSTPLTFGQGT | | |
| KVEIKRADAAPTVSIFPPSSEQLTSGGA | | |
| SVVCFLNNFYPKDINVKWKIDGSERQN | | |
| GVLNSWTDQDSKDSTYSMSSTLTLTK | | |
| DEYERHNSYTCEATHKTSTSPIVKSFN | | |
| RNEC | | |
| (SEQ ID NO: 1244) | | |
| DIQMTQSPSSLSASVGDRVTITCRASQS | SEQ ID NO: 693 | I23RB259 |
| ISNDLNWYQQKPGKAPKLLIYAASSLQ | SEQ ID NO: 767 | I23RB316 |
| SGVPSRFSGSGSGTDFTLTISSLQPEDF | | |
| ATYYCQQSYSTPLTFGQGTKVEIKRAD | | |
| AAPTVSIFPPSSEQLTSGGASVVCFLNN | | |
| FYPKDINVKWKIDGSERQNGVLNSWT | | |
| DQDSKDSTYSMSSTLTLTKDEYERHNS | | |
| YTCEATHKTSTSPIVKSFNRNEC | | |
| (SEQ ID NO: 1245) | | |
| EIVLTQSPGTLSLSPGERATLSCRASQS | SEQ ID NO: 709 | I23RB267 |
| VSSSYLAWYQQKPGQAPRLLIYGASSR | SEQ ID NO: 713 | I23RB269 |
| ATGIPDRESGSGSGTDFTLTISRLEPEDF | SEQ ID NO: 719 | I23RB272 |
| AVYYCQQYGSSPLTFGQGTKVEIKRA | SEQ ID NO: 731 | I23RB278 |
| DAAPTVSIFPPSSEQLTSGGASVVCFLN | SEQ ID NO: 735 | I23RB280 |
| NFYPKDINVKWKIDGSERQNGVLNSW | SEQ ID NO: 741 | I23RB283 |
| TDQDSKDSTYSMSSTLTLTKDEYERHN | SEQ ID NO: 749 | I23RB287 |
| SYTCEATHKTSTSPIVKSFNRNEC | SEQ ID NO: 751 | I23RB288 |
| (SEQ ID NO: 1246) | | |

TABLE 14
Shared Antibody VL Amino Acid Sequences

| | Corresponding Identical | Antibodies Comprising |
|---|---|---|
| VL Sequence and Consensus SEQ ID | Sequences and SEQ IDs | VL sequence |
| EIVLTQSPATLSLSPGERATLSCRASQSVSSY | SEQ ID NO: 787 | I23RB1 |
| LAWYQQKPGQAPRLLIYDASNRATGIPARFS | SEQ ID NO: 789 | I23RB2 |
| GSGSGTDFTLTISSLEPEDFAVYYCQQRSNW | SEQ ID NO: 791 | I23RB3 |
| PLTFGQGTKVEIK | SEQ ID NO: 793 | I23RB4 |
| (SEQ ID NO: 1247) | SEQ ID NO: 801 | I23RB8 |
| | SEQ ID NO: 803 | I23RB9 |
| | SEQ ID NO: 825 | I23RB268 |
| | SEQ ID NO: 829 | I23RB270 |
| | SEQ ID NO: 871 | I23RB291 |
| DIQMTQSPSSLSASVGDRVTITCRASQSISSY | SEQ ID NO: 795 | I23RB5 |
| LNWYQQKPGKAPKLLIYAASSLQSGVPSRFS | SEQ ID NO: 821 | I23RB266 |
| GSGSGTDFTLTISSLQPEDFATYYCQQSYSTP | SEQ ID NO: 831 | I23RB271 |
| LTFGQGTKVEIK | SEQ ID NO: 835 | I23RB273 |
| (SEQ ID NO: 1248) | SEQ ID NO: 837 | I23RB274 |
| | SEQ ID NO: 839 | I23RB275 |
| | SEQ ID NO: 841 | I23RB276 |
| | SEQ ID NO: 843 | I23RB277 |
| | SEQ ID NO: 847 | I23RB279 |
| | SEQ ID NO: 851 | I23RB281 |
| | SEQ ID NO: 853 | I23RB282 |
| | SEQ ID NO: 857 | I23RB284 |
| | SEQ ID NO: 859 | I23RB286 |
| | SEQ ID NO: 867 | I23RB289 |
| | SEQ ID NO: 869 | I23RB290 |
| | SEQ ID NO: 873 | I23RB292 |
| DIVMTQSPDSLAVSLGERATINCKSSQSVLY | SEQ ID NO: 797 | I23RB6 |
| SSNNKNYLAWYQQKPGQPPKLLIYWASTRE | SEQ ID NO: 805 | I23RB10 |
| SGVPDRFSGSGSGTDFTLTISSLQAEDVAVY | | |
| YCQQYYSTPLTFGQGTKVEIK (SEQ ID | | |
| NO: 1249) | | |
| DIQMTQSPSSLSASVGDRVTITCRASQSISND | SEQ ID NO: 807 | I23RB259 |
| LNWYQQKPGKAPKLLIYAASSLQSGVPSRFS | SEQ ID NO: 881 | I23RB316 |
| GSGSGTDFTLTISSLQPEDFATYYCQQSYSTP | | |
| LTFGQGTKVEIK | | |
| (SEQ ID NO: 1250) | | |
| EIVLTQSPGTLSLSPGERATLSCRASQSVSSS | SEQ ID NO: 823 | I23RB267 |
| YLAWYQQKPGQAPRLLIYGASSRATGIPDRF | SEQ ID NO: 827 | I23RB269 |
| SGSGSGTDFTLTISRLEPEDFAVYYCQQYGS | SEQ ID NO: 833 | I23RB272 |
| SPLTFGQGTKVEIK | SEQ ID NO: 845 | I23RB278 |
| (SEQ ID NO: 1251) | SEQ ID NO: 849 | I23RB280 |
| | SEQ ID NO: 855 | I23RB283 |
| | SEQ ID NO: 863 | I23RB287 |
| | SEQ ID NO: 865 | I23RB288 |

TABLE 15
Shared Antibody Light Chain CDR1, CDR2 and CDR3 Amino Acid Sequences

| CDR1, CDR2 and CDR3 | Corresponding Identical | Antibodies Comprising |
|---|---|---|
| Sequence and Consensus | CDR Sequences and | CDR1, CDR2 and |
| SEQ ID | SEQ IDs | CDR3 Sequences |
| CDR1 QSVSSY (SEQ ID | SEQ ID NO: 903 | I23RB1 |
| NO: 1252) | SEQ ID NO: 909 | I23RB2 |
| | SEQ ID NO: 915 | I23RB3 |
| | SEQ ID NO: 921 | I23RB4 |
| | SEQ ID NO: 945 | I23RB8 |
| | SEQ ID NO: 951 | I23RB9 |
| | SEQ ID NO: 1017 | I23RB268 |
| | SEQ ID NO: 1029 | I23RB270 |
| | SEQ ID NO: 1155 | I23RB291 |
| CDR2 DAS (SEQ ID | SEQ ID NO: 904 | |
| NO: 1253) | SEQ ID NO: 910 | |
| | SEQ ID NO: 916 | |
| | SEQ ID NO: 922 | |
| | SEQ ID NO: 946 | |
| | SEQ ID NO: 952 | |
| | SEQ ID NO: 1018 | |
| | SEQ ID NO: 1030 | |
| | SEQ ID NO: 1156 | |
| CDR3 QQRSNWPLT (SEQ ID | SEQ ID NO: 905 | |
| NO: 1254) | SEQ ID NO: 911 | |
| | SEQ ID NO: 917 | |
| | SEQ ID NO: 923 | |
| | SEQ ID NO: 947 | |
| | SEQ ID NO: 953 | |
| | SEQ ID NO: 1019 | |
| | SEQ ID NO: 1031 | |
| | SEQ ID NO: 1157 | |
| CDR1 QSISSY (SEQ ID | SEQ ID NO: 927 | I23RB5 |
| NO: 1255) | SEQ ID NO: 1005 | I23RB266 |
| | SEQ ID NO: 1035 | I23RB271 |
| | SEQ ID NO: 1047 | I23RB273 |
| | SEQ ID NO: 1053 | I23RB274 |
| | SEQ ID NO: 1059 | I23RB275 |
| | SEQ ID NO: 1065 | I23RB276 |
| | SEQ ID NO: 1071 | I23RB277 |
| | SEQ ID NO: 1083 | I23RB279 |
| | SEQ ID NO: 1095 | I23RB281 |
| | SEQ ID NO: 1101 | I23RB282 |
| | SEQ ID NO: 1113 | I23RB284 |
| | SEQ ID NO: 1125 | I23RB286 |
| | SEQ ID NO: 1143 | I23RB289 |
| | SEQ ID NO: 1149 | I23RB290 |
| | SEQ ID NO: 1161 | I23RB292 |
| CDR2 AAS (SEQ ID | SEQ ID NO: 928 | |
| NO: 1256) | SEQ ID NO: 1006 | |
| | SEQ ID NO: 1036 | |
| | SEQ ID NO: 1048 | |
| | SEQ ID NO: 1054 | |
| | SEQ ID NO: 1060 | |
| | SEQ ID NO: 1066 | |
| | SEQ ID NO: 1072 | |
| | SEQ ID NO: 1084 | |
| | SEQ ID NO: 1096 | |
| | SEQ ID NO: 1102 | |
| | SEQ ID NO: 1114 | |
| | SEQ ID NO: 1126 | |
| | SEQ ID NO: 1144 | |
| | SEQ ID NO: 1150 | |
| | SEQ ID NO: 1162 | |
| CDR3 QQSYSTPLT (SEQ ID | SEQ ID NO: 929 | |
| NO: 1257) | SEQ ID NO: 1007 | |
| | SEQ ID NO: 1037 | |
| | SEQ ID NO: 1049 | |
| | SEQ ID NO: 1055 | |
| | SEQ ID NO: 1061 | |
| | SEQ ID NO: 1067 | |
| | SEQ ID NO: 1073 | |
| | SEQ ID NO: 1085 | |
| | SEQ ID NO: 1097 | |
| | SEQ ID NO: 1103 | |
| | SEQ ID NO: 1115 | |
| | SEQ ID NO: 1127 | |
| | SEQ ID NO: 1145 | |
| | SEQ ID NO: 1151 | |
| | SEQ ID NO: 1163 | |
| CDR1 QSVLYSSNNKNY | SEQ ID NO: 933 | I23RB6 |
| (SEQ ID NO: 1258) | SEQ ID NO: 957 | I23RB10 |
| CDR2 WAS (SEQ ID | SEQ ID NO: 934 | |
| NO: 1259) | SEQ ID NO: 958 | |
| CDR3 QQYYSTPLT (SEQ ID | SEQ ID NO: 935 | |
| NO: 1260) | SEQ ID NO: 959 | |
| CDR1 QSISND (SEQ ID | SEQ ID NO: 963 | I23RB259 |
| NO: 1261) | SEQ ID NO: 1185 | I23RB316 |
| CDR2 AAS (SEQ ID | SEQ ID NO: 964 | |
| NO: 1262) | SEQ ID NO: 1186 | |
| CDR3 QQSYSTPLT (SEQ ID | SEQ ID NO: 965 | |
| NO: 1263) | SEQ ID NO: 1187 | |
| CDR1 QSVSSSY (SEQ ID | SEQ ID NO: 1011 | I23RB267 |
| NO: 1264) | SEQ ID NO: 1023 | I23RB269 |
| | SEQ ID NO: 1041 | I23RB272 |
| | SEQ ID NO: 1077 | I23RB278 |
| | SEQ ID NO: 1089 | I23RB280 |
| | SEQ ID NO: 1107 | I23RB283 |
| | SEQ ID NO: 1131 | I23RB287 |
| | SEQ ID NO: 1137 | I23RB288 |
| CDR2 GAS (SEQ ID | SEQ ID NO: 1012 | |
| NO: 1265) | SEQ ID NO: 1024 | |
| | SEQ ID NO: 1042 | |
| | SEQ ID NO: 1078 | |
| | SEQ ID NO: 1090 | |
| | SEQ ID NO: 1108 | |
| | SEQ ID NO: 1132 | |
| | SEQ ID NO: 1138 | |
| CDR3 QQYGSSPLT (SEQ ID | SEQ ID NO: 1013 | |
| NO: 1266) | SEQ ID NO: 1025 | |
| | SEQ ID NO: 1043 | |
| | SEQ ID NO: 1079 | |
| | SEQ ID NO: 1091 | |
| | SEQ ID NO: 1109 | |
| | SEQ ID NO: 1133 | |
| | SEQ ID NO: 1139 | |

1. An isolated monoclonal antibody or antigen binding fragment thereof that binds human interleukin-23 receptor (IL-23R) on a mammalian cell, wherein the antibody or fragment comprises:
a. a VH comprising an HCDR1, an HCDR2, and an HCDR3 having an amino acid sequence of SEQ ID NOs:570, 571, and 572, respectively, and a VL comprising an LCDR1, an LCDR2, and an LCDR3 having an amino acid sequence of SEQ ID NOs:573, 574, and 575, respectively;
b. a VH comprising an HCDR1, an HCDR2, and an HCDR3 having an amino acid sequence of SEQ ID NOs:936, 937, and 938, respectively, and a VL comprising an LCDR1, an LCDR2, and an LCDR3 having an amino acid sequence of SEQ ID NOs:939, 940, and 941, respectively;
c. a VH comprising an HCDR1, an HCDR2, and an HCDR3 having an amino acid sequence of SEQ ID NOs:918, 919, and 920, respectively, and a VL comprising an LCDR1, an LCDR2, and an LCDR3 having an amino acid sequence of SEQ ID NOs:921, 922, and 923, respectively;
d. a VH comprising an HCDR1, an HCDR2, and an HCDR3 having an amino acid sequence of SEQ ID NOs:900, 901, and 902, respectively, and a VL comprising an LCDR1, an LCDR2, and an LCDR3 having an amino acid sequence of SEQ ID NOs:903, 904, and 905, respectively;
e. a VH comprising an HCDR1, an HCDR2, and an HCDR3 having an amino acid sequence of SEQ ID NOs:276, 277, and 278, respectively, and a VL comprising an LCDR1, an LCDR2, and an LCDR3 having an amino acid sequence of SEQ ID NOs:279, 280, and 281, respectively;
f. a VH comprising an HCDR1, an HCDR2, and an HCDR3 having an amino acid sequence of SEQ ID NOs:294, 295, and 296, respectively, and a VL comprising an LCDR1, an LCDR2, and an LCDR3 having an amino acid sequence of SEQ ID NOs:297, 298, and 299, respectively;
g. a VH comprising an HCDR1, an HCDR2, and an HCDR3 having an amino acid sequence of SEQ ID NOs:318, 319, and 320, respectively, and a VL comprising an LCDR1, an LCDR2, and an LCDR3 having an amino acid sequence of SEQ ID NOs:321, 322, and 323, respectively;
h. a VH comprising an HCDR1, an HCDR2, and an HCDR3 having an amino acid sequence of SEQ ID NOs:324, 325, and 326, respectively, and a VL comprising an LCDR1, an LCDR2, and an LCDR3 having an amino acid sequence of SEQ ID NOs:327, 328, and 329, respectively;
i. a VH comprising an HCDR1, an HCDR2, and an HCDR3 having an amino acid sequence of SEQ ID NOs:330, 331, and 332, respectively, and a VL comprising an LCDR1, an LCDR2, and an LCDR3 having an amino acid sequence of SEQ ID NOs:333, 334, and 335, respectively;
j. a VH comprising an HCDR1, an HCDR2, and an HCDR3 having an amino acid sequence of SEQ ID NOs:342, 343, and 344, respectively, and a VL comprising an LCDR1, an LCDR2, and an LCDR3 having an amino acid sequence of SEQ ID NOs:345, 346, and 347, respectively;
k. a VH comprising an HCDR1, an HCDR2, and an HCDR3 having an amino acid sequence of SEQ ID NOs:354, 355, and 356, respectively, and a VL comprising an LCDR1, an LCDR2, and an LCDR3 having an amino acid sequence of SEQ ID NOs:357, 358, and 359, respectively;
l. a VH comprising an HCDR1, an HCDR2, and an HCDR3 having an amino acid sequence of SEQ ID NOs:360, 361, and 362, respectively, and a VL comprising an LCDR1, an LCDR2, and an LCDR3 having an amino acid sequence of SEQ ID NOs:363, 364, and 365, respectively;
m. a VH comprising an HCDR1, an HCDR2, and an HCDR3 having an amino acid sequence of SEQ ID NOs:366, 367, and 368, respectively, and a VL comprising an LCDR1, an LCDR2, and an LCDR3 having an amino acid sequence of SEQ ID NOs:369, 370, and 371, respectively;
n. a VH comprising an HCDR1, an HCDR2, and an HCDR3 having an amino acid sequence of SEQ ID NOs:384, 385, and 386, respectively, and a VL comprising an LCDR1, an LCDR2, and an LCDR3 having an amino acid sequence of SEQ ID NOs:387, 388, and 389, respectively;
o. a VH comprising an HCDR1, an HCDR2, and an HCDR3 having an amino acid sequence of SEQ ID NOs:906, 907, and 908, respectively, and a VL comprising an LCDR1, an LCDR2, and an LCDR3 having an amino acid sequence of SEQ ID NOs:909, 910, and 911, respectively;
p. a VH comprising an HCDR1, an HCDR2, and an HCDR3 having an amino acid sequence of SEQ ID NOs:420, 421, and 422, respectively, and a VL comprising an LCDR1, an LCDR2, and an LCDR3 having an amino acid sequence of SEQ ID NOs:423, 424, and 425, respectively;
q. a VH comprising an HCDR1, an HCDR2, and an HCDR3 having an amino acid sequence of SEQ ID NOs:1026, 1027, and 1028, respectively, and a VL comprising an LCDR1, an LCDR2, and an LCDR3 having an amino acid sequence of SEQ ID NOs:1029, 1030, and 1031, respectively;
r. a VH comprising an HCDR1, an HCDR2, and an HCDR3 having an amino acid sequence of SEQ ID NOs:912, 913, and 914, respectively, and a VL comprising an LCDR1, an LCDR2, and an LCDR3 having an amino acid sequence of SEQ ID NOs:915, 916, and 917, respectively;
s. a VH comprising an HCDR1, an HCDR2, and an HCDR3 having an amino acid sequence of SEQ ID NOs:426, 427, and 428, respectively, and a VL comprising an LCDR1, an LCDR2, and an LCDR3 having an amino acid sequence of SEQ ID NOs:429, 430, and 431, respectively;
t. a VH comprising an HCDR1, an HCDR2, and an HCDR3 having an amino acid sequence of SEQ ID NOs:1194, 1195, and 1196, respectively, and a VL comprising an LCDR1, an LCDR2, and an LCDR3 having an amino acid sequence of SEQ ID NOs:1197, 1198, and 1199, respectively;
u. a VH comprising an HCDR1, an HCDR2, and an HCDR3 having an amino acid sequence of SEQ ID NOs:438, 439, and 440, respectively, and a VL comprising an LCDR1, an LCDR2, and an LCDR3 having an amino acid sequence of SEQ ID NOs:441, 442, and 443, respectively;
v. a VH comprising an HCDR1, an HCDR2, and an HCDR3 having an amino acid sequence of SEQ ID NOs:444, 445, and 446, respectively, and a VL comprising an LCDR1, an LCDR2, and an LCDR3 having an amino acid sequence of SEQ ID NOs:447, 448, and 449, respectively;
w. a VH comprising an HCDR1, an HCDR2, and an HCDR3 having an amino acid sequence of SEQ ID NOs:456, 457, and 458, respectively, and a VL comprising an LCDR1, an LCDR2, and an LCDR3 having an amino acid sequence of SEQ ID NOs:459, 460, and 461, respectively;
x. a VH comprising an HCDR1, an HCDR2, and an HCDR3 having an amino acid sequence of SEQ ID NOs:462, 463, and 464, respectively, and a VL comprising an LCDR1, an LCDR2, and an LCDR3 having an amino acid sequence of SEQ ID NOs:465, 466, and 467, respectively;
y. a VH comprising an HCDR1, an HCDR2, and an HCDR3 having an amino acid sequence of SEQ ID NOs:468, 469, and 470, respectively, and a VL comprising an LCDR1, an LCDR2, and an LCDR3 having an amino acid sequence of SEQ ID NOs:471, 472, and 473, respectively;
z. a VH comprising an HCDR1, an HCDR2, and an HCDR3 having an amino acid sequence of SEQ ID NOs:474, 475, and 476, respectively, and a VL comprising an LCDR1, an LCDR2, and an LCDR3 having an amino acid sequence of SEQ ID NOs:477, 478, and 479, respectively;
aa. a VH comprising an HCDR1, an HCDR2, and an HCDR3 having an amino acid sequence of SEQ ID NOs:480, 481, and 482, respectively, and a VL comprising an LCDR1, an LCDR2, and an LCDR3 having an amino acid sequence of SEQ ID NOs:483, 484, and 485, respectively;
bb. a VH comprising an HCDR1, an HCDR2, and an HCDR3 having an amino acid sequence of SEQ ID NOs:510, 511, and 512, respectively, and a VL comprising an LCDR1, an LCDR2, and an LCDR3 having an amino acid sequence of SEQ ID NOs:513, 514, and 515, respectively;
cc. a VH comprising an HCDR1, an HCDR2, and an HCDR3 having an amino acid sequence of SEQ ID NOs:528, 529, and 530, respectively, and a VL comprising an LCDR1, an LCDR2, and an LCDR3 having an amino acid sequence of SEQ ID NOs:531, 532, and 533, respectively;
dd. a VH comprising an HCDR1, an HCDR2, and an HCDR3 having an amino acid sequence of SEQ ID NOs:552, 553, and 554, respectively, and a VL comprising an LCDR1, an LCDR2, and an LCDR3 having an amino acid sequence of SEQ ID NOs:555, 556, and 557, respectively;
ee. a VH comprising an HCDR1, an HCDR2, and an HCDR3 having an amino acid sequence of SEQ ID NOs:558, 559, and 560, respectively, and a VL comprising an LCDR1, an LCDR2, and an LCDR3 having an amino acid sequence of SEQ ID NOs:561, 562, and 563, respectively;
ff. a VH comprising an HCDR1, an HCDR2, and an HCDR3 having an amino acid sequence of SEQ ID NOs:564, 565, and 566, respectively, and a VL comprising an LCDR1, an LCDR2, and an LCDR3 having an amino acid sequence of SEQ ID NOs:567, 568, and 569, respectively;
gg. a VH comprising an HCDR1, an HCDR2, and an HCDR3 having an amino acid sequence of SEQ ID NOs:576, 577, and 578, respectively, and a VL comprising an LCDR1, an LCDR2, and an LCDR3 having an amino acid sequence of SEQ ID NOs:579, 580, and 581, respectively;
hh. a VH comprising an HCDR1, an HCDR2, and an HCDR3 having an amino acid sequence of SEQ ID NOs:600, 601, and 602, respectively, and a VL comprising an LCDR1, an LCDR2, and an LCDR3 having an amino acid sequence of SEQ ID NOs:603, 604, and 605, respectively;
ii. a VH comprising an HCDR1, an HCDR2, and an HCDR3 having an amino acid sequence of SEQ ID NOs:606, 607, and 608, respectively, and a VL comprising an LCDR1, an LCDR2, and an LCDR3 having an amino acid sequence of SEQ ID NOs:609, 610, and 611, respectively;
jj. a VH comprising an HCDR1, an HCDR2, and an HCDR3 having an amino acid sequence of SEQ ID NOs:624, 625, and 626, respectively, and a VL comprising an LCDR1, an LCDR2, and an LCDR3 having an amino acid sequence of SEQ ID NOs:627, 628, and 629, respectively;
kk. a VH comprising an HCDR1, an HCDR2, and an HCDR3 having an amino acid sequence of SEQ ID NOs:642, 643, and 644, respectively, and a VL comprising an LCDR1, an LCDR2, and an LCDR3 having an amino acid sequence of SEQ ID NOs:645, 646, and 647, respectively;
ll. a VH comprising an HCDR1, an HCDR2, and an HCDR3 having an amino acid sequence of SEQ ID NOs:648, 649, and 650, respectively, and a VL comprising an LCDR1, an LCDR2, and an LCDR3 having an amino acid sequence of SEQ ID NOs:651, 652, and 653, respectively; or
mm. a VH comprising an HCDR1, an HCDR2, and an HCDR3 having an amino acid sequence of SEQ ID NOs:654, 655, and 656, respectively, and a VL comprising an LCDR1, an LCDR2, and an LCDR3 having an amino acid sequence of SEQ ID NOs:657, 658, and 659, respectively.

2. The isolated monoclonal antibody or antigen binding fragment thereof of claim 1 that binds human interleukin-23 receptor (IL-23R) on a mammalian cell, wherein the antibody or fragment comprises:
a. a VH comprising the amino acid sequence of SEQ ID NO:236, and a VL comprising the amino acid sequence of SEQ ID NO:237;
b. a VH comprising the amino acid sequence of SEQ ID NO:798, and a VL comprising the amino acid sequence of SEQ ID NO:799;
c. a VH comprising the amino acid sequence of SEQ ID NO:792, and a VL comprising the amino acid sequence of SEQ ID NO:793;
d. a VH comprising the amino acid sequence of SEQ ID NO:786, and a VL comprising the amino acid sequence of SEQ ID NO:787;
e. a VH comprising the amino acid sequence of SEQ ID NO:138, and a VL comprising the amino acid sequence of SEQ ID NO:139;
f. a VH comprising the amino acid sequence of SEQ ID NO:144, and a VL comprising the amino acid sequence of SEQ ID NO:145;
g. a VH comprising the amino acid sequence of SEQ ID NO:152, and a VL comprising the amino acid sequence of SEQ ID NO:153;
h. a VH comprising the amino acid sequence of SEQ ID NO:154, and a VL comprising the amino acid sequence of SEQ ID NO:155;
i. a VH comprising the amino acid sequence of SEQ ID NO:156, and a VL comprising the amino acid sequence of SEQ ID NO:157;
j. a VH comprising the amino acid sequence of SEQ ID NO:160, and a VL comprising the amino acid sequence of SEQ ID NO:161;
k. a VH comprising the amino acid sequence of SEQ ID NO:164, and a VL comprising the amino acid sequence of SEQ ID NO:165;
l. a VH comprising the amino acid sequence of SEQ ID NO:166, and a VL comprising the amino acid sequence of SEQ ID NO:167;
m. a VH comprising the amino acid sequence of SEQ ID NO:168, and a VL comprising the amino acid sequence of SEQ ID NO:169;
n. a VH comprising the amino acid sequence of SEQ ID NO:174, and a VL comprising the amino acid sequence of SEQ ID NO:175;
o. a VH comprising the amino acid sequence of SEQ ID NO:788, and a VL comprising the amino acid sequence of SEQ ID NO:789;
p. a VH comprising the amino acid sequence of SEQ ID NO:186, and a VL comprising the amino acid sequence of SEQ ID NO:187;
q. a VH comprising the amino acid sequence of SEQ ID NO:828, and a VL comprising the amino acid sequence of SEQ ID NO:829;
r. a VH comprising the amino acid sequence of SEQ ID NO:790, and a VL comprising the amino acid sequence of SEQ ID NO:791;
S. a VH comprising the amino acid sequence of SEQ ID NO:188, and a VL comprising the amino acid sequence of SEQ ID NO:189;
t. a VH comprising the amino acid sequence of SEQ ID NO:884, and a VL comprising the amino acid sequence of SEQ ID NO:885;
u. a VH comprising the amino acid sequence of SEQ ID NO:192, and a VL comprising the amino acid sequence of SEQ ID NO:193;
v. a VH comprising the amino acid sequence of SEQ ID NO:194, and a VL comprising the amino acid sequence of SEQ ID NO:195;
w. a VH comprising the amino acid sequence of SEQ ID NO:198, and a VL comprising the amino acid sequence of SEQ ID NO:199;
x. a VH comprising the amino acid sequence of SEQ ID NO:200, and a VL comprising the amino acid sequence of SEQ ID NO:201;
y. a VH comprising the amino acid sequence of SEQ ID NO:202, and a VL comprising the amino acid sequence of SEQ ID NO:203;
z. a VH comprising the amino acid sequence of SEQ ID NO:204, and a VL comprising the amino acid sequence of SEQ ID NO:205;
aa. a VH comprising the amino acid sequence of SEQ ID NO:206, and a VL comprising the amino acid sequence of SEQ ID NO:207;
bb. a VH comprising the amino acid sequence of SEQ ID NO:216, and a VL comprising the amino acid sequence of SEQ ID NO:217;
cc. a VH comprising the amino acid sequence of SEQ ID NO:222, and a VL comprising the amino acid sequence of SEQ ID NO:223;
dd. a VH comprising the amino acid sequence of SEQ ID NO:230, and a VL comprising the amino acid sequence of SEQ ID NO:231;
ee. a VH comprising the amino acid sequence of SEQ ID NO:232, and a VL comprising the amino acid sequence of SEQ ID NO:233;
ff. a VH comprising the amino acid sequence of SEQ ID NO:234, and a VL comprising the amino acid sequence of SEQ ID NO:235;
gg. a VH comprising the amino acid sequence of SEQ ID NO:238, and a VL comprising the amino acid sequence of SEQ ID NO:239;
hh. a VH comprising the amino acid sequence of SEQ ID NO:246, and a VL comprising the amino acid sequence of SEQ ID NO:247;
ii. a VH comprising the amino acid sequence of SEQ ID NO:248, and a VL comprising the amino acid sequence of SEQ ID NO:249;
jj. a VH comprising the amino acid sequence of SEQ ID NO:254, and a VL comprising the amino acid sequence of SEQ ID NO:255;
kk. a VH comprising the amino acid sequence of SEQ ID NO:260, and a VL comprising the amino acid sequence of SEQ ID NO:261;
ll. a VH comprising the amino acid sequence of SEQ ID NO:262, and a VL comprising the amino acid sequence of SEQ ID NO:263; or
mm. a VH comprising the amino acid sequence of SEQ ID NO:264, and a VL comprising the amino acid sequence of SEQ ID NO:265.

3. The isolated monoclonal antibody or antigen binding fragment thereof of claim 1 that binds human interleukin-23 receptor (IL-23R) on a mammalian cell, wherein the antibody or fragment comprises:
a. a VH comprising an HCDR1, and HCDR2 and an HCDR3 having an amino acid sequence of SEQ ID NOs:570, 571, and 572, respectively and a VL comprising an LCDR1, an LCDR2, and an LCDR3 having an amino acid sequence of SEQ ID NOs:573, 574, and 575, respectively;
b. a VH comprising an HCDR1, and HCDR2 and an HCDR3 having an amino acid sequence of SEQ ID NOs:936, 937, and 938, respectively and a VL comprising an LCDR1, an LCDR2, and an LCDR3 having an amino acid sequence of SEQ ID NOs:939, 940, and 941, respectively;
c. a VH comprising an HCDR1, and HCDR2 and an HCDR3 having an amino acid sequence of SEQ ID NOs:918, 919, and 920, respectively and a VL comprising an LCDR1, an LCDR2, and an LCDR3 having an amino acid sequence of SEQ ID NOs:921, 922, and 923, respectively;
d. a VH comprising an HCDR1, and HCDR2 and an HCDR3 having an amino acid sequence of SEQ ID NOs:900, 901, and 902, respectively and a VL comprising an LCDR1, an LCDR2, and an LCDR3 having an amino acid sequence of SEQ ID NOs:903, 904, and 905, respectively; or
e. a VH comprising an HCDR1, and HCDR2 and an HCDR3 having an amino acid sequence of SEQ ID NOs:912, 913, and 914, respectively and a VL comprising an LCDR1, an LCDR2, and an LCDR3 having an amino acid sequence of SEQ ID NOs:915, 916, and 917, respectively.

4. The isolated monoclonal antibody or antigen binding fragment thereof of claim 2 that binds human interleukin-23 receptor (IL-23R) on a mammalian cell, wherein the antibody or fragment comprises:
a. a VH comprising the amino acid sequence of SEQ ID NO:236, and a VL comprising the amino acid sequence of SEQ ID NO:237;
b. a VH comprising the amino acid sequence of SEQ ID NO:798, and a VL comprising the amino acid sequence of SEQ ID NO:799;
c. a VH comprising the amino acid sequence of SEQ ID NO:792, and a VL comprising the amino acid sequence of SEQ ID NO:793;
d. a VH comprising the amino acid sequence of SEQ ID NO:786, and a VL comprising the amino acid sequence of SEQ ID NO:787; or
e. a VH comprising the amino acid sequence of SEQ ID NO:790, and a VL comprising the amino acid sequence of SEQ ID NO:791.

5. (canceled)

6. (canceled)

7. An isolated monoclonal antibody or antigen binding fragment thereof which recognizes human and rat interleukin-23 receptor (IL-23R), wherein the antibody or fragment comprises:
a. a VH comprising an HCDR1, and HCDR2 and an HCDR3 having an amino acid sequence of SEQ ID NOs:468, 469, and 470, respectively and a VL comprising an LCDR1, an LCDR2, and an LCDR3 having an amino acid sequence of SEQ ID NOs:471, 472, and 473, respectively;
b. a VH comprising an HCDR1, and HCDR2 and an HCDR3 having an amino acid sequence of SEQ ID NOs:618, 619, and 620, respectively and a VL comprising an LCDR1, an LCDR2, and an LCDR3 having an amino acid sequence of SEQ ID NOs:621, 622, and 623, respectively;
c. a VH comprising an HCDR1, and HCDR2 and an HCDR3 having an amino acid sequence of SEQ ID NOs:330, 331, and 332, respectively and a VL comprising an LCDR1, an LCDR2, and an LCDR3 having an amino acid sequence of SEQ ID NOs:333, 334, and 335, respectively;
d. a VH comprising an HCDR1, and HCDR2 and an HCDR3 having an amino acid sequence of SEQ ID NOs:936,937, and 938, respectively and a VL comprising an LCDR1, an LCDR2, and an LCDR3 having an amino acid sequence of SEQ ID NOs:939, 940, and 941, respectively;
e. a VH comprising an HCDR1, and HCDR2 and an HCDR3 having an amino acid sequence of SEQ ID NOs:312, 313, and 314, respectively and a VL comprising an LCDR1, an LCDR2, and an LCDR3 having an amino acid sequence of SEQ ID NOs:315, 316, and 317, respectively;
f. a VH comprising an HCDR1, and HCDR2 and an HCDR3 having an amino acid sequence of SEQ ID NOs:474, 475, and 476, respectively and a VL comprising an LCDR1, an LCDR2, and an LCDR3 having an amino acid sequence of SEQ ID NOs:477, 478, and 479, respectively;
g. a VH comprising an HCDR1, and HCDR2 and an HCDR3 having an amino acid sequence of SEQ ID NOs:1194, 1195, and 1196, respectively and a VL comprising an LCDR1, an LCDR2, and an LCDR3 having an amino acid sequence of SEQ ID NOs:1197, 1198, and 1199,
h. a VH comprising an HCDR1, and HCDR2 and an HCDR3 having an amino acid sequence of SEQ ID NOs:426, 427, and 428, respectively and a VL comprising an LCDR1, an LCDR2, and an LCDR3 having an amino acid sequence of SEQ ID NOs:429, 430, and 431, respectively; or
i. a VH comprising an HCDR1, and HCDR2 and an HCDR3 having an amino acid sequence of SEQ ID NOs:498, 499, and 500, respectively and a VL comprising an LCDR1, an LCDR2, and an LCDR3 having an amino acid sequence of SEQ ID NOs:501, 502, and 503, respectively.

8. The isolated monoclonal antibody or antigen binding fragment thereof of claim 7 which recognizes human and rat interleukin-23 receptor (IL-23R), wherein the antibody or fragment comprises:
a. a VH comprising the amino acid sequence of SEQ ID NO:202, and a VL comprising the amino acid sequence of SEQ ID NO:203;
b. a VH comprising the amino acid sequence of SEQ ID NO:252, and a VL comprising the amino acid sequence of SEQ ID NO:253;
c. a VH comprising the amino acid sequence of SEQ ID NO:156, and a VL comprising the amino acid sequence of SEQ ID NO:157;
d. a VH comprising the amino acid sequence of SEQ ID NO:798, and a VL comprising the amino acid sequence of SEQ ID NO:799;
e. a VH comprising the amino acid sequence of SEQ ID NO:150, and a VL comprising the amino acid sequence of SEQ ID NO:151;
f. a VH comprising the amino acid sequence of SEQ ID NO:204, and a VL comprising the amino acid sequence of SEQ ID NO:205;
g. a VH comprising the amino acid sequence of SEQ ID NO:884, and a VL comprising the amino acid sequence of SEQ ID NO:885;
h. a VH comprising the amino acid sequence of SEQ ID NO:188, and a VL comprising the amino acid sequence of SEQ ID NO:189; or
i. a VH comprising the amino acid sequence of SEQ ID NO:212, and a VL comprising the amino acid sequence of SEQ ID NO:213.

9. An isolated monoclonal antibody or antigen binding fragment thereof that binds interleukin-23 receptor (IL-23R) comprising a VL comprising CDR sequences of an LCDR1 SEQ ID NO:1252, an LCDR2 SEQ ID NO:1253, and an LCDR3 SEQ ID NO:1254 and a VH comprising CDR sequences selected from:
a. an HCDR1 SEQ ID NO:900, an HCDR2 SEQ ID NO:901 and an HCDR3 SEQ ID NO:902;
b. an HCDR1 SEQ ID NO:906, an HCDR2 SEQ ID NO:907, and an HCDR3 SEQ ID NO:908;
c. an HCDR1 SEQ ID NO:912, an HCDR2 SEQ ID NO:913, and an HCDR3 SEQ ID NO:914;
d. an HCDR1 SEQ ID NO:918, an HCDR2 SEQ ID NO:919, and an HCDR3 SEQ ID NO:920;
e. an HCDR1 SEQ ID NO:942, an HCDR2 SEQ ID NO:943, and an HCDR3 SEQ ID NO:944;
f. an HCDR1 SEQ ID NO:948, an HCDR2 SEQ ID NO:949, and an HCDR3 SEQ ID NO:950;
g. an HCDR1 SEQ ID NO:1014, an HCDR2 SEQ ID NO:1015, and an HCDR3 SEQ ID NO:1016;
h. an HCDR1 SEQ ID NO:1026, an HCDR2 SEQ ID NO:1027, and an HCDR3 SEQ ID NO:1028; and
i. an HCDR1 SEQ ID NO:1152, an HCDR2 SEQ ID NO:1153, and an HCDR3 SEQ ID NO:1154.

10. The isolated monoclonal antibody or antigen binding fragment thereof of claim 9 comprising a VL comprising CDR sequences of an LCDR1 SEQ ID NO:1252, an LCDR2 SEQ ID NO:1253, and an LCDR3 SEQ ID NO:1254 or having the amino acid sequence SEQ ID NO:1247 and a VH sequence selected from SEQ ID NO:786, 788, 790, 792, 800, 802, 824, 828 and 870.

11-21. (canceled)

22. The antibody or fragment of claim 1, wherein the antibody is an IgG.

23. The antibody or fragment of claim 1, wherein the antibody is a humanized or chimeric antibody or fragment thereof.

24. The isolated antibody or fragment of claim 1 which is an antibody or fragment thereof wherein the isolated antibody is a form of an antibody F(ab′)2, scFv fragment, domain antibody, minibody, diabody, triabody or tetrabody.

25. (canceled)

26. (canceled)

27. (canceled)

28. (canceled)

29. A composition comprising the antibody of claim 1, and a pharmaceutically acceptable excipient.

30-44. (canceled)

45. A method of treating a disease or disorder in a subject, comprising administering to the subject the composition of claim 29, wherein the disease or disorder is an IL-23R associated disease or disorder.

46. The method according to claim 45, wherein the disease or disorder is selected from multiple sclerosis, asthma, rheumatoid arthritis, inflammation of the gut, inflammatory bowel diseases (IBDs), juvenile IBD, adolescent IBD, Crohn's disease, ulcerative colitis, Celiac disease (nontropical Sprue), microscopic colitis, collagenous colitis, eosinophilic gastroenteritis/esophagitis, colitis associated with radio- or chemo-therapy, colitis associated with disorders of innate immunity as in leukocyte adhesion deficiency-1, sarcoidosis, Systemic Lupus Erythematosus, ankylosing spondylitis (axial spondyloarthritis), psoriatic arthritis, psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, enteropathy associated with seronegative arthropathies, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

47. (canceled)

48. (canceled)
